(19) United States
(12) Patent Application Publication
(10) Pub. No.: US 2026/0347319 A1
(43) Pub. Date: Nov. 5, 2026

(54) YAP/TAZ-TEAD ONCOPROTEINS INHIBITORS

(71) Applicant: BridGene Biosciences, Inc., San Jose, CA (US)

(72) Inventors: Henry William Beecroft JOHNSON, San Jose, CA (US); Zhushou LUO, San Jose, CA (US); Chao ZHANG, San Jose, CA (US); Michael J. BISHOP, San Jose, CA (US)

(21) Appl. No.: 19/112,072

(22) PCT Filed: Sep. 15, 2023
(86) PCT No.: PCT/US2023/032957

§ 371 Date: Mar. 14, 2025

Related U.S. Application Data
(60) Provisional application No. 63/407,148, filed on Sep. 15, 2022.

(51) Int. Cl. C07D 205/04; A61K 31/397; A61K 31/40; A61K 31/422; C07D 207/12; C07D 413/06

(52) U.S. Cl. CPC C07D 205/04; A61K 31/397; A61K 31/40; A61K 31/422; C07D 207/12; C07D 413/06

(57) ABSTRACT

Provided are YAP/TAZ-TEAD oncoproteins inhibitors of Formula (I), or a stereoisomer, and/or a pharmaceutically acceptable salt, and/or solvate thereof, pharmaceutical compositions thereof, and methods of treatment.

### FIELD OF THE INVENTION

The present invention relates generally to cancer therapy, and more specifically to the synthesis of covalent inhibitors of YAP/TAZ-TEAD oncoproteins and use thereof for the treatment of cancers.

### BACKGROUND

TEA domain transcription factors (TEAD proteins) are effectors for oncogenic YAP/TAZ signaling in cancers with deregulated Hippo tumor suppressor pathway signaling. The interaction between Yes1 Associated Transcriptional Regulator (YAP1, aka YAP), WW domain-containing Transcription Regulator 1 (WWTR1, aka TAZ), a transcriptional coactivator paralog to YAP, and TEAD proteins was demonstrated both in vitro and in vivo. In both cases, the interaction of the proteins leads to increased TEAD transcriptional activity and expression of genes involved in proliferation, survival, angiogenesis, and other hallmarks of cancer. In normal tissues, intact Hippo signaling via MST1/2 and LATS1/2 kinases results in phosphorylation of YAP/TAZ, sequestration in the cytoplasm and proteosomal degradation. Inactivation of Hippo signaling as a result of somatic mutations in NF2, MST or LATS stabilizes YAP/TAZ and leads to nuclear translocation, association with TEAD, and activation of TEAD target genes. Genomic amplification of YAP/TAZ, or gene fusions resulting in constitutively active YAP/TAZ, are alternative mechanisms of Hippo pathway deregulation. In addition, activation of YAP/TAZ signaling has been described as a mechanism of resistance to other targeted cancer therapies, such as inhibitors of EGFR and MEK.

Accordingly, inhibitors of TEAD1, 2, 3 and/or 4, can have great potential in the application of cancer therapy, both as monotherapy in cancers with deregulated Hippo pathway signaling and as combination therapy with other targeted agents.

### SUMMARY

The present application is based on the discovery of a class of small-molecule compounds effective as covalent inhibitors of YAP/TAZ-TEAD oncoproteins. These compounds can be used in cancer therapy.

In a first aspect, provided is a compound according to Formula (I):

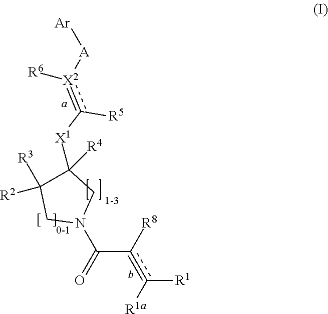

or a stereoisomer, and/or a pharmaceutically acceptable salt, and/or solvate thereof, wherein:
bond b is a double bond or triple bond;
R¹ is selected from the group consisting of H, C₁₋₃alkyl, morpholin-4-yl, and —CH₂N(C₁₋₃alkyl)₂;
R^{1a} is absent when bond b is a triple bond; and R^{1a} is H when bond b is a double bond;
R² is selected from the group consisting of H, F, —OH, C₁₋₃alkyl, —OC₁₋₃alkyl, —NH-heteroaryl, heteroaryl, —NH-(substituted heteroaryl), and substituted heteroaryl;
R³ is selected from the group consisting of H and C₁₋₃alkyl; and
R⁸ is absent when bond b is a triple bond; and R⁸ is selected from the group consisting of H, F, CN, and C₁₋₃alkyl when bond b is a double bond; and
i) wherein bond a, X¹, X², R⁴, R⁵, R⁶, A, and Ar are as follows:
   bond a is a double bond or triple bond;
   X¹ is absent or is selected from the group consisting of O, CH(OH), CH(OCH₃), CH₂, CH₂CH₂, CH(CH₃), and C(CH₃)₂, and R⁴ is selected from the group consisting of H, C₁-C₄ alkyl, cyclopropyl, F, Cl, —OH, —OC₁₋₃alkyl, CN, and NHR⁷, and R⁷ is selected from the group consisting of H and C₁-C₃ alkyl; or X¹ and R⁴ together with the carbon to which they are attached form C₄-C₆ cycloalkylene;
   X² is X^{2a} and X^{2a} is C;
   R⁵ is absent when bond a is a triple bond; or R⁵ is selected from the group consisting of H, F, and C₁-C₃ alkyl when bond a is a double bond.
   R⁶ is absent when bond a is a triple bond; or R⁶ is selected from the group consisting of H, F, and C₁-C₃ alkyl when bond a is a double bond;
   A is absent or selected from the group consisting of O, CH₂, CH₂CH₂, CH(OH), CH(OCH₃), C(CH₃)₂ and CH(CH₃); and
   Ar is aryl or heteroaryl, each of which is optionally substituted with 1, 2, 3, or 4 R⁹ groups; or
ii) wherein bond a, X¹, X², R⁴, R⁵, R⁶, A, and Ar together form:

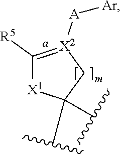

and
   X² is X^{2a} and X^{2a} is C; R⁴ and R⁶ together form —(CH₂)ₘ—; m is 0, 1, or 2; X¹ is absent or is selected from the group consisting of CH₂, CH₂CH₂, CH(CH₃), O, CH₂O, and OCH₂; R⁵ is selected from the group consisting of H, F, and C₁-C₃ alkyl; A is absent or selected from the group consisting of O, CH₂, CH₂CH₂, and CH(CH₃); and Ar is aryl or heteroaryl, each of which is optionally substituted with 1, 2, 3, or 4 R⁹ groups; or
iii) wherein bond a, X¹, X², R⁴, R⁵, R⁶, A, and Ar together form:

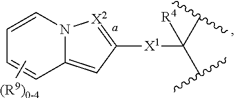

and
   X¹ is absent or is selected from the group consisting of O, CH₂, CH₂CH₂, and CH(CH₃); X² is X^{2b} and X^{2b} is N; R⁴ is selected from the group consisting of H, C₁-C₄ alkyl, cyclopropyl, F, Cl, —OH, —OC₁₋₃alkyl, CN, and NHR⁷, and R⁷ is selected from the group consisting of H and C₁-C₃ alkyl; R⁵ and Ar together form

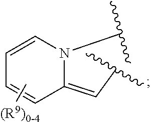

and R⁶ and A are absent; or
iv) wherein bond a, X¹, X², R⁴, R⁵, R⁶, A, and Ar together form:

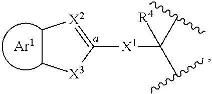

and
   X¹ is absent or is selected from the group consisting of O, CH₂, CH₂CH₂, and CH(CH₃); X² is X^{2c} and X^{2c} is N or CR⁶, and R⁶ is selected from the group consisting of H, F, and C₁-C₃ alkyl; R⁴ is selected from the group consisting of H, C₁-C₄ alkyl, cyclopropyl, F, Cl, —OH, —OC₁₋₃alkyl, CN, and NHR⁷, and R⁷ is selected from the group consisting of H and C₁-C₃ alkyl; A is absent; and R⁵ and Ar together form

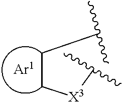

where X³ is O, NH, N(C₁-C₃ alkyl), and Ar¹ is a fused arylene or fused heteroarylene ring, where the Ar¹ ring is optionally substituted with 1, 2, 3, or 4 R⁹ groups; and
wherein each R⁹ is independently selected from the group consisting of C₁-C₆ alkyl, C₂-C₆ alkenyl, C₂-C₆ alkynyl, halo, C₁-C₆ haloalkyl, C₁-C₆ alkoxy, and CN; and
provided that the compound is not 1-[4-[(1E)-2-(2,6-dimethylphenyl)ethenyl]-1-piperidinyl]-2-propen-1-one.

In a second aspect, the present disclosure provides a pharmaceutical composition, comprising a pharmaceutically effective amount of the compound of the present application (including according to formula (I), (II), any embodiments herein, and any of Compounds 1-106) or a stereoisomer, and/or pharmaceutically acceptable salt, and/or solvate thereof, and a pharmaceutically acceptable carrier.

In a third aspect, a method of treatment of a disease or disorder modulated by TEAD1, 2, 3, and/or 4 is provided comprising administering to an individual in need thereof a therapeutically or prophylactically effective amount of a compound disclosed herein (including of Formula (I), (II), any embodiments, and any of compounds 1-106), or administering to an individual in need thereof a pharmaceutical composition comprising a therapeutically or prophylactically effective amount of a compound disclosed herein (including of Formula (I), (II), any embodiments, and any of compounds 1-106), and a pharmaceutically acceptable carrier. In some or any embodiments, the method is that wherein TEAD1 is inhibited selectively over TEAD2, 3, and 4. In some or any embodiments, the disease or disorder is selected from cancer, fibrotic diseases, neurofibromatosis type 2, and polycystic kidney disease.

In a fourth aspect, the present disclosure provides a method of treating cancer in a subject in need thereof, comprising administering to the subject a therapeutically effective amount of the compound disclosed herein (including according to formula (I), (II), any embodiments herein, and any of Compounds 1-106), or a stereoisomer, and/or a pharmaceutically acceptable salt, and/or solvate thereof, or a pharmaceutical composition a therapeutically effective amount of the compound disclosed herein, or a stereoisomer, and/or a pharmaceutically acceptable salt, and/or solvate thereof and a pharmaceutically acceptable carrier.

In a fifth aspect, provided herein is a compound useful as an intermediate in the preparation of the compounds disclosed herein.

In a sixth aspect, provided herein is a method of preparing a compound of Formula (I) according to any of the general schemes or synthetic examples.

In another aspect, the disclosure provides a method of inhibiting TEAD1, 2, 3, and/or 4 in a subject or in a sample comprising administering a compound of Formula (I) (including according to formula (I), (II), any embodiments herein, and any of Compounds 1-106) or a stereoisomer and/or a pharmaceutically acceptable salt thereof, and/or a solvate thereof. In certain embodiments, the compound is at least one of the compounds as shown in Table 1 or a stereoisomer, and/or a pharmaceutically acceptable salt thereof, and/or a solvate thereof.

### BRIEF DESCRIPTION OF THE FIGURES

FIG. 1 is a schematic illustration of how the disclosed compounds function as covalent TEAD1 inhibitors.

FIG. 2 is a graph illustrating the percent of body weight changes in mice over time after treatment according to Example 5.

FIG. 3 is a graph illustrating the percent of volume changes in mice tumors over time after treatment according to Example 5

### DETAILED DESCRIPTION

The present disclosure is based on the discovery of a class of small-molecule compounds that inhibits TEAD1, 2, 3, and/or 4 activity, in some embodiments selectively inhibits the activity of TEAD1 over that of the other TEAD isoforms, which exhibits potential in the treatment of cancer, and in some embodiments, certain types of cancers. TEAD1 belongs to TEADs family, which regulates cell growth and proliferation via TEAD1/YAP/TAZ complex. TEAD1 regulates glioblastoma stemness and invasiveness by regulating EGFR and AQP4 expression, via the pathway of TEAD-EGFR/AQP4. Further via the pathway of Hippo-YAP/TAZ-TEAD, YAP/TAZ-TEAD activation induces transcription of cell cycle-promoting genes; TEAD1 regulates expression of cytoskeleton remodeling gene; TEAD1 increases expression of other transcription factor like Myc and SP1; YAP/TAZ-TEAD1 axis regulates cell apoptosis; blocking TEAD1 aberrant activities will down regulates gene expression of MYC, KRAS, BRAF, NF2, LKB1 and PD-L1, etc. As a result, inhibitor compounds that inhibit TEAD1, 2, 3, and/or 4 activity (in some embodiments, selectively for TEAD1) have potential in the treatment of cancers such as glioblastoma, gastric cancer, colorectal cancer, pancreatic ductal adenocarcinoma (PDAC), and malignant pleural mesothelioma (MPM).

It is to be understood that the aspects and embodiments disclosed herein are not limited to particular compositions, methods, and experimental conditions described, as such compositions, methods, and conditions may vary. It is also to be understood that the terminology used herein is for purposes of describing particular embodiments only, and is not intended to be limiting.

Although any methods and materials similar or equivalent to those described herein can be used in the practice or testing of the invention, it will be understood that modifications and variations are encompassed within the spirit and scope of the instant disclosure. The preferred methods and materials are now described.

All publications, patents, and patent applications mentioned in this specification are herein incorporated by reference to the same extent as if each individual publication, patent, or patent application was specifically and individually indicated to be incorporated by reference.

### Definitions

As used in this specification and the appended claims, the singular forms “a,” “an,” and “the” include plural references unless the context clearly dictates otherwise. Thus, for example, references to “the method” includes one or more methods, and/or steps of the type described herein which will become apparent to those persons skilled in the art upon reading this disclosure.

In the event that there is a plurality of definitions for a term herein, those in this section prevail unless stated otherwise. Unless specified otherwise, where a term is defined as being substituted, the groups in the list of substituents are themselves unsubstituted. For example, a substituted alkyl group can be substituted, for example, with a cycloalkyl group, and the cycloalkyl group is not further substituted unless specified otherwise.

When referring to the compounds provided herein, the following terms have the following meanings unless indicated otherwise. Unless defined otherwise, all technical and scientific terms used herein have the same meaning as commonly understood by one of ordinary skill in the art to which this invention belongs.

The term “about” will be understood by persons of ordinary skill in the art. Whether the term “about” is used explicitly or not, every quantity given herein refers to the actual given value, and it is also meant to refer to the approximation to such given value that would be reasonably inferred based on the ordinary skill in the art. Reference to “about” a value or parameter herein includes (and describes) variations that are directed to that value or parameter per se. For example, description referring to “about X” includes description of “X”. As used herein, and unless otherwise specified, the terms “about” and “approximately,” when used in connection with temperatures, doses, amounts, or weight percent of ingredients of a composition or a dosage form, mean a dose, amount, or weight percent that is recognized by those of ordinary skill in the art to provide a pharmacological effect equivalent to that obtained from the specified dose, amount, or weight percent. Specifically, the terms “about” and “approximately,” when used in this context, contemplate a dose, amount, or weight percent within 15%, within 10%, within 5%, within 4%, within 3%, within 2%, within 1%, or within 0.5% of the specified dose, amount, or weight percent.

“Acyl,” as used herein, refers to a —C(O)R group where R is alkyl or cycloalkyl, as defined herein.

“Alkyl” refers to a linear or branched, monovalent, saturated hydrocarbon, where the alkyl has 1 to 12 carbon atoms, and in some embodiments 1 to 10 carbons, in some embodiments 1 to 6 carbons, in some embodiments 1 to 3 carbons, or in some embodiments having 1, 2, 3 or 4 carbon atoms. Examples of alkyl groups include, but are not limited to, methyl, ethyl, n-propyl, isopropyl, n-butyl, isobutyl, sec-butyl, tert-butyl n-pentyl, and n-hexyl groups. “Lower alkyl” means an alkyl group having one to six carbon atoms. “C₀” alkyl (as in “C₀-C₆-alkyl”) is a covalent bond. “C₆ alkyl” refers to, for example, n-hexyl, iso-hexyl, and the like. In some embodiments, alkyl is C₁₋₃alkyl or C₁₋₄alkyl.

“Alkylcarbonyl” refers to a —C(O)R group where R is alkyl, as defined herein. In some embodiments, acyl is acetyl. Lower alkylcarbonyl is where the alkyl is C₁-C₆alkyl.

“Alkylcarbonyloxy” refers to a —OC(O)R group where R is alkyl, as defined herein.

“Alkylsulfonyl” refers to a —S(O)₂R group where R is alkyl, as defined herein.

“C₁-C₆Alkylthio” refers to an —SR group, where R is alkyl, as defined herein.

“Alkoxy” refers to an —OR group, where R is alkyl, as defined herein. Exemplary alkoxy groups include, but are not limited to, methoxy, ethoxy, propoxy, butoxy, pentoxy, hexoxy, isopropoxy, sec-butoxy, tert-butoxy, cyclopropyloxy, cyclobutyloxy, cyclopentyloxy, and cyclohexyloxy groups. In some embodiments, alkoxy is methoxy. “Lower alkoxy” means an —OR, where R is alkyl having one to six carbon atoms.

“C₁-C₆alkoxycarbonyl” refers to a —C(O)R group where R is C₁-C₆alkoxy, as defined herein,

“Haloalkyl” refers to an alkyl group, as defined herein, substituted with one or more halogens, for example one, two, three, four, or five halo atoms. Representative examples includes 2,2-difluoroethyl, trifluoromethyl, and 2-chloro-1-fluoroethyl, and the like. “Lower haloalkyl” means the alkyl has one to six carbon atoms. In some embodiments, the haloalkyl is trifluoromethyl. “Lower perhaloalkyl” means that every hydrogen in the alkyl is replaced with a halo.

“C₁-C₆haloalkylthio” refers to an —SR group where R is a C₁-C₆haloalkyl group as defined herein.

“Haloalkenyl” refers to an alkenyl group, as defined herein, substituted with one or more halogens, for example one, two, three, four, or five halo atoms. “Lower haloalkenyl” means the alkenyl has two to six carbon atoms.

“Haloalkynyl” refers to an alkynyl group, as defined herein, substituted with one or more halogens, for example one, two, three, four, or five halo atoms. “Lower haloalkynyl” means the alkynyl has two to six carbon atoms.

“Haloalkoxy” refers to an —OR group where R is haloalkyl, as defined herein. “Lower perhaloalkoxy” means that every hydrogen in the alkyl is replaced with a halo.

“Cycloalkyl” refers to a monocyclic or bicyclic, monovalent group hydrocarbon having three to fourteen carbon ring atoms. Cycloalkyl is saturated or partially saturated but cannot contain an aromatic ring. Cycloalkyl includes fused, bridged, and spiro ring systems, In some embodiments, cycloalkyl has from 3 to 12, 3 to 10, 3 to 8, 4 to 6, or 3, 4, 5, 6 or 7 carbon atoms. Examples of monocyclic cycloalkyl includes, but are not limited to cyclopropyl, cyclobutyl, cyclopentyl, and cyclohexyl groups. Examples of bicyclic cycloalkyl include, but are not limited to, bicyclo[4.4.0]decanyl, bicyclo[2.2.1]heptanyl, spiro[2.2]pentanyl, and the like.

“Cycloalkylene” refers to a divalent cycloalkyl, a defined herein. Examples of monocyclic cycloalkylene include, but are not limited to cycloprop-diyl, cyclobut-diyl, cyclopent-diyl, and cyclohex-diyl groups. Examples of bicyclic cycloalkylene include, but are not limited to, bicyclo[4.4.0]decan-diyl, bicyclo[2.2.1]heptan-diyl, spiro[2.2]pentan-diyl, and the like. In some embodiments, cycloalkylene is C₄-C₆ cycloalkylene.

“Alkenyl” refers to a linear or branched, momvalent hydrocarbon radical comprising 2 to 12 carbon atoms and one or more double bonds between two carbon atoms. In some embodiments, alkenyl has from 2 to about 10 carbon atoms, in some embodiments 2 to 8 carbon atoms, in some embodiments from 2 to about 6 carbon atoms, or in some embodiments 2 2, 3 or 4 carbon atoms Examples of alkenyl groups include, but are not limited to, vinyl, allyl, —CH═CH(CH₃), —CH═C(CH₃)₂, —C(CH₃)═CH₂, cyclopentenyl, cyclohexenyl, butadienyl, pentadienyl, and hexadienyl, among others. “Lower alkenyl” means an alkenyl group having two to six carbon atoms.

“Alkynyl” refers to a straight or branched hydrocarbon radical, with one or more triple bonds between two carbon atoms, and 2 to 12 carbon atoms, in some embodiments from 2 to 10 carbon atoms, in some embodiments from 2 to 6 carbon atoms, or 2, 3 or in some embodiments 4 carbon atoms Exemplary alkynyl groups include, but are not limited to, ethynyl, propargyl, and —C≡C(CH₃), among others. “Lower alkynyl” means an alkynyl group having two to six carbon atoms.

“Aryl” means a monovalent six- to fourteen-membered, mono- or bi-carbocyclic ring, wherein the monocyclic ring is aromatic and at least one of the rings in the bicyclic ring is aromatic. In some embodiments, the aryl contains 6 to 10 carbon ring atoms. Representative examples include phenyl, naphthyl, and indanyl, and the like. In some embodiments, aryl is phenyl.

“Heteroaryl” refers to a monocyclic, fused bicyclic, or fused tricyclic, monovalent radical of 5 to 14 ring atoms containing one or more heteroatoms, for example one, two, three, or four ring heteroatoms, independently selected from N, —N(R^{x})—, O, S, and S(O)ₙ— (n is 0, 1, or 2) and the remaining ring atoms being carbon, wherein the ring comprising a monocyclic radical is aromatic and wherein at least one of the fused rings comprising a bicyclic or tricyclic radical is aromatic. One or two ring carbon atoms of any nonaromatic rings comprising a bicyclic or tricyclic radical may be replaced by a —C(O)—, —C(S)—, or —C(═NH)— group. R^{x} is hydrogen, alkyl, hydroxy, alkoxy, acyl, or alkylsulfonyl. Fused bicyclic radical includes bridged ring systems. Unless stated otherwise, the point of attachment may be located on any atom of any ring of the heteroaryl group, valency rules permitting. In particular, when the point of attachment is located on the nitrogen, R^{x} is absent. Heteroaryl includes, but is not limited to, isoxazolyl, oxazolyl, oxadiazolyl, pyridyl, pyrazinyl, pyrimidinyl, pyridazinyl, thiazolyl, pyrazolyl, 1,2,4-triazolyl, 1,3,5-triazolyl, tetrazoyl, pyrrolyl, imidazolyl, thienyl, furanyl, indolyl, 2,3-dihydro-1H-indolyl (including, for example, 2,3-dihydro-1H-indol-2-yl or 2,3-dihydro-1H-indol-5-yl, and the like), isoindolyl, indolinyl, isoindolinyl, phthalimidyl, pyrazolopyridyl, benzofuranyl, benzimidazolyl, benzodioxol-4-yl, cinnolinyl, indolizinyl, naphthyridin-3-yl, phthalazin-3-yl, phthalazin-4-yl, pteridinyl, purinyl, quinazolinyl, quinoxalinyl, benzoxazolyl, quinolinyl, isoquinolinyl, tetrahydroisoquinolinyl (including, for example, tetrahydroisoquinolin-4-yl or tetrahydroisoquinolin-6-yl, and the like), pyrrolo[3,2-c]pyridinyl (including, for example, pyrrolo[3,2-c]pyridin-2-yl or pyrrolo[3,2-c]pyridin-7-yl, and the like), benzopyranyl, thiazolyl, isothiazolyl, thiadiazolyl, benzothiazolyl, benzothienyl. In some embodiments, heteroaryl is a 5-membered or 6-membered heteroaryl. In some embodiments, heteroaryl is pyrazolyl, oxazolyl, isoxazolyl, thiazolyl, pyrimidinyl, or pyridinyl. In some embodiments, heteroaryl is pyrazolyl, pyrimidinyl, or pyridinyl. In some embodiments, heteroaryl is pyrazolyl or pyrimidinyl. In some embodiments, heteroaryl is oxazolyl, isoxazolyl, thiazolyl, or pyridinyl.

“Fused arylene” refers to a divalent, monocyclic aryl ring, as defined herein, which is fused to second ring, and is represented by Ar¹ portion in the following ring:

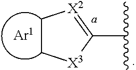

In some embodiments, the Ar¹ fused arylene is

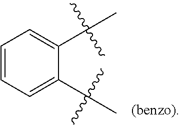 [SMILES: *c1ccccc1*]

“Fused heteroarylene” refers to a divalent, monocyclic heteroaryl ring, as defined herein, which is fused to second ring, and is represented by Ar¹ portion in the following ring:

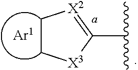

In some embodiments, the Ar¹ fused heteroarylene is

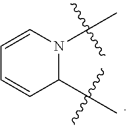 [SMILES: *C1C=CC=CN1*]

“Heterocycloalkyl” means a saturated monovalent monocyclic group of 3 to 9 ring atoms or a saturated monovalent fused bicyclic group of 5 to 12 ring atoms in which one or more heteroatoms, for example one, two, three, or four ring heteroatoms, independently selected from —O—, —S(O)ₙ— (n is 0, 1, or 2), —N═, —N(R^{y})— (where R^{y} is hydrogen, alkyl, hydroxy, alkoxy, acyl, or alkylsulfonyl), the remaining ring atoms being carbon. One or two ring carbon atoms may be replaced by a —C(O)—, —C(S)—, or —C(═NH)— group. Fused bicyclic radical includes bridged ring systems. Unless otherwise stated, the point of attachment of the group may be located on any atom of any ring within the radical, valency rules permitting. In particular, when the point of attachment is located on a nitrogen atom, R^{y} is absent. More specifically the term heterocycloalkyl includes, but is not limited to, azetidinyl, pyrrolidinyl, 2-oxopyrrolidinyl, 2,5-dihydro-1H-pyrrolyl, piperidinyl, 4-piperidonyl, morpholinyl, piperazinyl, 2-oxopiperazinyl, tetrahydropyranyl, 2-oxopiperidinyl, thiomorpholinyl, thiamorpholinyl, perhydroazepinyl, pyrazolidinyl, imidazolinyl, imidazolidinyl, dihydropyridinyl, tetrahydropyridinyl, oxazolinyl, oxazolidinyl, isoxazolidinyl, thiazolinyl, thiazolidinyl, quinuclidinyl, isothiazolidinyl, octahydroindolyl, octahydroisoindolyl, decahydroisoquinolyl, tetrahydrofuryl, and tetrahydropyranyl, and an N-oxide thereof.

Halogen groups include F, Cl, Br, and I; nitro group refers to —NO₂; and cyano group refers to —CN.

“Amino” refers to —NH₂.

“Lower carbamate” refers to —O(C═O)NRₐR_{b}, where Rₐ and R_{b} are independently hydrogen, C₁-C₆alkyl, aryl, or heterocyclyl.

The term “substituted heteroaryl” refers to heteroaryl, as defined herein, substituted with one or more (1, 2, 3, or 4) substituents independently selected from the following groups: lower alkyl, lower alkenyl, lower alkynyl, C₃-C₆heterocycloalkyl, lower haloalkyl, lower haloalkenyl, lower haloalkynyl, lower perhaloalkyl, lower perhaloalkoxy, C₃-C₆cycloalkyl, phenyl, lower alkoxy, lower haloalkoxy, oxo, lower alkylcarbonyloxy, lower alkylcarbonyl, C₁-C₆alkoxycarbonyl, —C(O)NH₂, —C(O)NH(C₁-C₆alkyl), —C(O)N(C₁-C₆alkyl)₂, cyano, halogen, hydroxy, amino, lower alkylamino, —NHC(O)R (where R is C₁-C₆alkyl), nitro, C₁-C₆alkylthio, C₁-C₆haloalkylthio, SH, and lower carbamate.

The term “protecting group,” as used herein, and unless otherwise specified, refers to a group that is added to an oxygen, nitrogen or phosphorus atom to prevent its further reaction or for other purposes. A wide variety of oxygen and nitrogen protecting groups are known to those skilled in the art of organic synthesis. (See for example those described in Greene, et al., Protective Groups in Organic Synthesis, John Wiley and Sons, Fourth Edition, 2006, hereby incorporated by reference.) In some or any embodiments, a “nitrogen-protecting group” is 9-fluorenylmethyloxycarbonyl (Fmoc), tert-butoxycarbonyl (Boc), benzyloxycarbonyl (CBz), acetyl, trichloroacetyl, trifluoroacetyl, —C(O)OCH₂CCl₃ (Troc), p-methoxyphenyl, benzyl, p-methoxybenzyl, p-methoxybenzylcarbonyl, triphenylmethyl, benzylidenyl, 2,2,2-trichloroethoxysulfonyl (Tces), p-methoxybenzenesulfonyl (Mbs) or p-toluenesulfonyl (tosyl). In some or any embodiments, an oxygen-protecting group (e.g. for X¹) is methoxymethyl (MOM), ethoxyethyl, methoxyethoxymethyl, tetrahydrofuranyl, tetrahydropyranyl, methyl, tert-butyl, allyl, benzyl, trimethylsilyl, triethylsilyl, triisopropylsilyl, tert-butyldimethylsilyl, tert-butyldiphenylsilyl, acetyl, pivalyl, benzoyl, dimethoxytrityl, trityl, methoxytrityl, p-methoxybenzyl, or methylthiomethyl.

“Stereoisomer” refers to compounds which have the same molecular formula and sequence of bonded atoms (constitution), but differ in the three-dimensional orientations of their atoms in space. Stereoisomer includes enantiomers, diastereomers, geometric isomers (e.g., regioisomers, cis and trans isomers, Z and E isomers), and atropisomers. Enantiomers are a pair of molecules with the exact same connectivity that exist in two forms that are mirror images of one another but cannot be superimposed one upon the other. Diastereoisomers are non-identical stereoisomers which have different configurations at one or more of the equivalent stereocenters and are not mirror images of each other. Geometric isomers include cis-trans isomers or configurational isomers, and refer to pairs of molecules which have the same formula but whose functional groups are in different orientations in three-dimensional space. cis-trans Stereoisomers contain double bonds that do not rotate, or they may contain ring structures, where the rotation of bonds is restricted or prevented. cis indicates that the functional groups (substituents) are on the same side of some plane, while trans conveys that they are on opposing (transverse) sides. Atropisomers are stereoisomers resulting from hindered rotation about single bonds where the steric strain barrier to rotation is high enough to allow for the isolation of the conformers. In some or any embodiments, the stereoisomer is a geometric isomer. In some or any embodiments, the stereoisomer is an enantiomer. In some or any embodiments, the stereoisomer is an enantiomer and geometric isomer.

The term “substantially free of” or “substantially in the absence of” stereoisomers (for example, geometric isomers) with respect to a composition refers to a composition that includes at least 85 or 90% by weight, in some or any embodiments 95%, 98%, 99% or 100% by weight, of a designated stereoisomer (for example, geometric isomer) of a compound in the composition. In some or any embodiments, in the methods and compounds provided herein, the compounds are substantially free of stereoisomers (for example, geometric isomers) of the designated compound.

Similarly, the term “isolated” with respect to a composition refers to a composition that includes at least 85, 90%, 95%, 98%, 99% to 100% by weight, of a specified compound, the remainder comprising other chemical species, or stereoisomers (for example, geometric isomers) thereof.

The term “solvate,” as used herein, and unless otherwise specified, refers to a compound provided herein or a salt thereof, that further includes a stoichiometric or non-stoichiometric amount of solvent bound by non-covalent intermolecular forces. Where the solvent is water, the solvate is a hydrate.

Pharmaceutically acceptable salts of compounds described herein (including according to formula (I), (II), any embodiments herein, and any of Compounds 1-106) include conventional nontoxic salts or quaternary ammonium salts of a compound, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like. In other cases, described compounds may contain one or more acidic functional groups and, thus, are capable of forming pharmaceutically acceptable salts with pharmaceutically acceptable bases. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like.

### Compounds

The aspects and embodiments described herein include the recited compounds as well as a pharmaceutically acceptable salt, and/or hydrate, and/or solvate, and/or stereoisomer, and/or tautomer, and/or mixture, or any combination thereof. In some embodiments, the recited compounds are provided as a pharmaceutically acceptable salt, and/or stereoisomer, and/or tautomer, and/or mixture, or any combination thereof. In some embodiments, the recited compounds are provided as a pharmaceutically acceptable salt, and/or stereoisomer, and/or tautomer thereof. In some embodiments, the recited compounds are provided as a pharmaceutically acceptable salt thereof.

Certain multicyclic structures provided herein are drawn with one or more floating substituents. Unless provided otherwise or otherwise clear from the context, the substituent(s) may be present on any atom of the multicyclic ring, where chemically feasible and valency rules permitting. For example, in the structure:

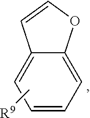

the R⁹ substituents can be on the benzo portion of the bicyclic ring or the furanyl portion of the bicyclic ring.

Embodiment A: In some or any embodiments, the compound of Formula (I) is that wherein

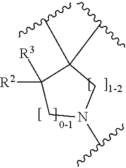

is

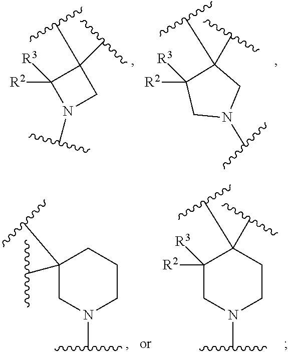

bond b is a double bond or triple bond;
R¹ is H or C₁₋₃alkyl (in some embodiments, methyl);
R^{1a} is absent when bond b is a triple bond; and R^{1a} is H when bond b is a double bond;
R² is selected from the group consisting of H, C₁₋₂alkyl, methoxy, —NH-heteroaryl (in some embodiments, —NH-pyrimidinyl), and 5-membered heteroaryl (in some embodiments, pyrazolyl);
R³ is H or methyl; and
R⁸ is absent when bond b is a triple bond; and R⁸ is selected from the group consisting of H and F when bond b is a double bond; and
i) wherein bond a, X¹, X², R⁴, R⁵, R⁶, A, and Ar are as follows:
   bond a is a double bond or triple bond;
   X¹ is absent, CH₂, CH(OH), or C(CH₃)₂, and R⁴ is selected from the group consisting of H, methyl, F, —OH, methoxy, and CN; or X¹ and R⁴ together with the carbon to which they are attached form C₄-C₆ cycloalkylene;
   X² is X^{2a} and X^{2a} is C;
   R⁵ is absent when bond a is a triple bond; or R⁵ is H, when bond a is a double bond.
   R⁶ is absent when bond a is a triple bond; or R⁶ is H, when bond a is a double bond;
   A is absent or is selected from the group consisting of CH₂, CH(OH), CH(OCH₃), and C(CH₃)₂; and
   Ar is phenyl, oxazolyl, thiazolyl, or pyridinyl, each of which is optionally substituted with 1 or 2 R⁹ groups; or
ii) wherein bond a, X¹, X², R⁴, R⁵, R⁶, A, and Ar together form:

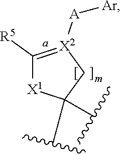

and
   X² is X^{2a} and X^{2a} is C; R⁴ and R⁶ together form —(CH₂)ₘ—; m is 0, 1, or 2; X¹ is absent or is selected from the group consisting of CH₂, O, CH₂O and OCH₂; R⁵ is H; A is absent; and Ar is phenyl optionally substituted with 1 R⁹ group; provided that m is 1 or 2 when X¹ is absent; or
iii) wherein bond a, X¹, X², R⁴, R¹, R⁶, A, and Ar together form:

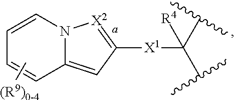

and
   X¹ is absent; X² is X^{2b} and X^{2b} is N; R⁴ is H; R⁵ and Ar together form

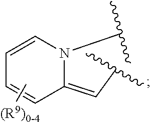

and R⁶ and A are absent; or
iv) wherein bond a, X¹, X², R⁴, R⁵, R⁶, A, and Ar together form:

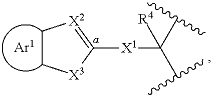

and
   X¹ is absent; X² is X^{2c} and X^{2c} is CR⁶, and R⁶ is of H; R⁴ is H; A is absent; and R⁵ and Ar together form

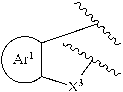

where X³ is O, NH, N(C₁-C₃ alkyl), and Ar¹ is a fused phenylene optionally substituted with 1 R⁹; and
wherein each R⁹ is independently selected from the group consisting of ethynyl, F, —CF₃, and methoxy; and
provided that the compound is not 1-[4-[(1E)-2-(2,6-dimethylphenyl)ethenyl]-1-piperidinyl]-2-propen-1-one.

Embodiment B: In some or any embodiments, the compound of Formula (I) is that wherein

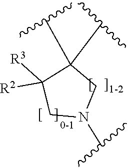

is

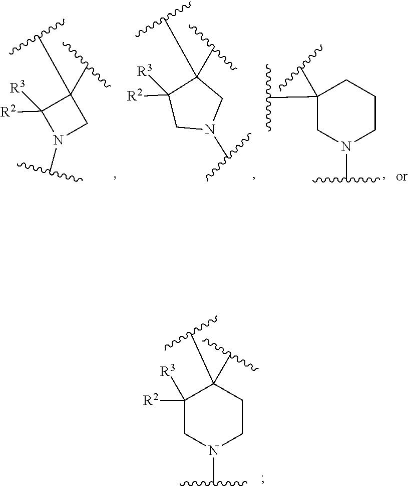

bond b is a double bond or triple bond;
R¹ is H;
R^{1a} is absent when bond b is a triple bond; and R^{1a} is H when bond b is a double bond;
R² is selected from the group consisting of H, C₁₋₂alkyl, methoxy, and 5-membered heteroaryl (in some embodiments, pyrazolyl);
R³ is H or methyl; and
R⁸ is absent when bond b is a triple bond; and R⁸ is selected from the group consisting of H, and F when bond b is a double bond; and
i) wherein bond a, X¹, X², R⁴, R⁵, R⁶, A, and Ar are as follows:
   bond a is a double bond or triple bond;
   X¹ is absent or is CH₂, and R⁴ is selected from the group consisting of H, methyl, F, —OH, methoxy, and CN; or X¹ and R⁴ together with the carbon to which they are attached form C₄-C₆ cycloalkylene;
   X² is X^{2a} and X^{2a} is C;
   R⁵ is absent when bond a is a triple bond; or R⁵ is H, when bond a is a double bond;
   R⁶ is absent when bond a is a triple bond; or R⁶ is H, when bond a is a double bond;
   A is absent; and
   Ar is phenyl, oxazolyl, thiazolyl, or pyridinyl, each of which is optionally substituted with 1 or 2 R⁹ groups; or
ii) wherein bond a, X¹, X², R⁴, R⁵, R⁶, A, and Ar together form:

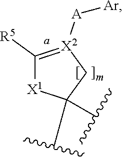

and
   X² is X^{2a} and X^{2a} is C; R⁴ and R⁶ together form —(CH₂)ₘ—; m is 1 or 2; X¹ is absent or is selected from the group consisting of CH₂; R⁵ is selected from the group consisting of H; A is absent; and Ar is phenyl, optionally substituted with 1 R⁹ group; or
   wherein bond a, X¹, X², R⁴, R⁵, R⁶, A, and Ar together form:

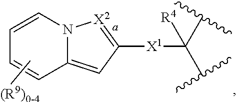

and
   X¹ is absent; X² is X^{2b} and X^{2b} is N; R⁴ is selected from the group consisting of H; R⁵ and Ar together form

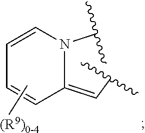

and R⁶ and A are absent; or
iii) wherein bond a, X¹, X², R⁴, R⁵, R⁶, A, and Ar together form:

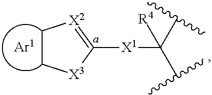

and
   X¹ is absent; X² is X^{2c} and X^{2c} is CR⁶, and R⁶ is selected from the group consisting of H; R⁴ is H; A is absent; and R⁵ and Ar together form

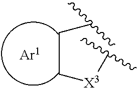

where X³ is O, NH, N(C₁-C₃ alkyl), and Ar¹ is a fused phenylene, optionally substituted with 1 R⁹; and
wherein each R⁹ is independently selected from the group consisting of ethynyl, F, —CF₃, and methoxy; and
provided that the compound is not 1-[4-[(1E)-2-(2,6-dimethylphenyl)ethenyl]-1-piperidinyl]-2-propen-1-one.

Embodiment 1: Provided is a compound of formula (I), or a stereoisomer, and/or a pharmaceutically acceptable salt, and/or solvate thereof, as provided in the first aspect. In some or any embodiments, the compound of formula (I) is according to Embodiment A or B.

Embodiment 1A: Provided is a compound of formula (I),
or a stereoisomer, and/or a pharmaceutically acceptable salt, and/or solvate thereof,
wherein:
bond b is a double bond or triple bond;
R¹ is selected from the group consisting of H, C₁₋₃alkyl, morpholin-4-yl, and —CH₂N(C₁₋₃alkyl)₂;
R^{1a} is absent when bond b is a triple bond; and R^{1a} is H when bond b is a double bond;
R² is selected from the group consisting of H, F, —OH, C₁₋₃alkyl, —OC₁₋₃alkyl, —NH-heteroaryl, heteroaryl, —NH-(substituted heteroaryl), and substituted heteroaryl;
R³ is selected from the group consisting of H, and C₁₋₃alkyl; and
R⁸ is absent when bond b is a triple bond; and R⁸ is selected from the group consisting of H, F, CN, and C₁₋₃alkyl when bond b is a double bond; and
i) wherein bond a, X¹, X², R⁴, R⁵, R⁶, A, and Ar are as follows:
   bond a is a double bond or triple bond;
   X¹ is absent or is selected from the group consisting of O, CH₂, CH₂CH₂, and CH(CH₃), and R⁴ is selected from the group consisting of H, C₁-C₄ alkyl, cyclopropyl, F, Cl, —OH, —OC₁₋₃alkyl, CN, and NHR⁷, and R⁷ is selected from the group consisting of H and C₁-C₃ alkyl; or X¹ and R⁴ together with the carbon to which they are attached form C₄-C₆ cycloalkylene;
   X² is X^{2a} and X^{2a} is C;
   R⁵ is absent when bond a is a triple bond; or R⁵ is selected from the group consisting of H, F, and C₁-C₃ alkyl when bond a is a double bond.
   R⁶ is absent when bond a is a triple bond; or R⁶ is selected from the group consisting of H, F, and C₁-C₃ alkyl when bond a is a double bond;
   A is absent or selected from the group consisting of O, CH₂, CH₂CH₂, and CH(CH₃); and
   Ar is aryl or heteroaryl group, optionally substituted with 1, 2, 3, or 4 R⁹ groups; or
ii) wherein bond a, X¹, X², R⁴, R⁵, R⁶, A, and Ar together form:

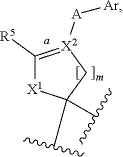

and
   X² is X^{2a} and X^{2a} is C; R⁴ and R⁶ together form —(CH₂)ₘ—; m is 1 or 2; X¹ is absent or is selected from the group consisting of CH₂, CH₂CH₂, and CH(CH₃); R⁵ is selected from the group consisting of H, F, and C₁-C₃ alkyl; A is absent or selected from the group consisting of O, CH₂, CH₂CH₂, and CH(CH₃); and Ar is aryl or heteroaryl group, optionally substituted with 1, 2, 3, or 4 R⁹ groups; or
iii) wherein bond a, X¹, X², R⁴, R⁵, R⁶, A, and Ar together form:

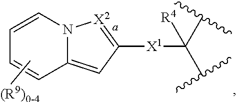

and
   X¹ is absent or is selected from the group consisting of O, CH₂, CH₂CH₂, and CH(CH₃); X² is X^{2b} and X^{2b} is N; R⁴ is selected from the group consisting of H, C₁-C₄ alkyl, cyclopropyl, F, Cl, —OH, —OC₁₋₃alkyl, CN, and NHR⁷, and R⁷ is selected from the group consisting of H and C₁-C₃ alkyl; R⁵ and Ar together form

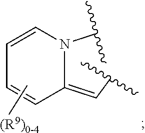

and R⁶ and A are absent; or
iv) wherein bond a, X¹, X², R⁴, R⁵, R⁶, A, and Ar together form:

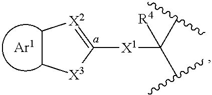

and
   X¹ is absent or is selected from the group consisting of O, CH₂, CH₂CH₂, and CH(CH₃); X² is X^{2c} and X^{2c} is N or CR⁶, and R⁶ is selected from the group consisting of H, F, and C₁-C₃ alkyl; R⁴ is selected from the group consisting of H, C₁-C₄ alkyl, cyclopropyl, F, Cl, —OH, —OC₁₋₃alkyl, CN, and NHR⁷, and R⁷ is selected from the group consisting of H and C₁-C₃ alkyl; A is absent; and R⁵ and Ar together form

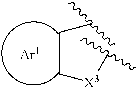

where X³ is O, NH, N(C₁-C₃ alkyl), and Ari is a fused arylene or fused heteroarylene ring, optionally substituted with 1, 2, 3, or 4 R⁹ groups; and
wherein each R⁹ is independently selected from the group consisting of C₁-C₆ alkyl, C₂-C₆ alkenyl, C₂-C₆ alkynyl, halo, C₁-C₆ haloalkyl, C₁-C₆ alkoxy, and CN; and
provided that the compound is not 1-[4-[(1E)-2-(2,6-dimethylphenyl)ethenyl]-1-piperidinyl]-2-propen-1-one.

Embodiment 2. In embodiment 2, provided is a compound of any one of embodiments 1, 1A, A, and B, wherein bond b is a double bond and R^{1a} is H; or a stereoisomer, and/or a pharmaceutically acceptable salt, and/or solvate thereof.

Embodiment 3. In embodiment 3, provided is a compound of any one of embodiments 1, 1A, A, and B, wherein bond b is a triple bond and R^{1a} and R⁸ are absent; or a stereoisomer, and/or a pharmaceutically acceptable salt, and/or solvate thereof.

Embodiment 4. In embodiment 4, provided is a compound of any one of embodiments A and 1-3, wherein R¹ is H; or a stereoisomer, and/or a pharmaceutically acceptable salt, and/or solvate thereof.

Embodiment 5. In embodiment 5, provided is a compound of any one of embodiments A and 1-3, wherein R¹ is Me; or a stereoisomer, and/or a pharmaceutically acceptable salt, and/or solvate thereof

Embodiment 6. In embodiment 6, provided is a compound of any one of embodiments 1-3, wherein R¹ is —CH₂N(Me)₂; or a stereoisomer, and/or a pharmaceutically acceptable salt, and/or solvate thereof

Embodiment 7a. In embodiment 7a, provided is a compound of any one of embodiments A and 1-3, wherein R¹ is selected from the group consisting of H and C₁₋₃alkyl; or a stereoisomer, and/or a pharmaceutically acceptable salt, and/or solvate thereof.

Embodiment 7. In embodiment 7, provided is a compound of any one of embodiments A, 1-3 and 7a, wherein R¹ is selected from the group consisting of H and Me; or a stereoisomer, and/or a pharmaceutically acceptable salt, and/or solvate thereof.

Embodiment 8. In embodiment 8, provided is a compound of any one of embodiments A, B, and 1-7, wherein R² is H; or a stereoisomer, and/or a pharmaceutically acceptable salt, and/or solvate thereof.

Embodiment 9. In embodiment 9, provided is a compound of any one of embodiments 1-7, wherein R² is F; or a stereoisomer, and/or a pharmaceutically acceptable salt, and/or solvate thereof.

Embodiment 10. In embodiment 10, provided is a compound of any one of embodiments 1-7, wherein R² is —OH; or a stereoisomer, and/or a pharmaceutically acceptable salt, and/or solvate thereof.

Embodiment 11. In embodiment 11, provided is a compound of any one of embodiments 1-7, wherein R² is C₁₋₃alkyl; or a stereoisomer, and/or a pharmaceutically acceptable salt, and/or solvate thereof. In embodiment 11, provided is a compound of any one of embodiments A, B, and 1-7, wherein R² is C₁₋₂alkyl; or a stereoisomer, and/or a pharmaceutically acceptable salt, and/or solvate thereof.

Embodiment 12. In embodiment 12, provided is a compound of any one of embodiments A, B, and 1-7, wherein R² is H, methyl or ethyl; or a stereoisomer, and/or a pharmaceutically acceptable salt, and/or solvate thereof.

Embodiment 13a. In embodiment 2, provided is a compound of any one of embodiments 1-7, wherein R² is —OC₁₋₃alkyl; or a stereoisomer, and/or a pharmaceutically acceptable salt, and/or solvate thereof.

Embodiment 13. In embodiment 13, provided is a compound of any one of embodiments A. B, and 1-7, wherein R² is —OMe; or a stereoisomer, and/or a pharmaceutically acceptable salt, and/or solvate thereof.

Embodiment 14. In embodiment 14, provided is a compound of any one of embodiments 1-7, wherein R¹ is —NH-heteroaryl or —NH-(substituted heteroaryl); or a stereoisomer, and/or a pharmaceutically acceptable salt, and/or solvate thereof.

Embodiment 15. In embodiment 15, provided is a compound of any one of embodiments 1-7, wherein R² is unsubstituted heteroaryl (in some embodiments, pyrazolyl) or substituted heteroaryl (in some embodiments, pyrazolyl); or a stereoisomer, and/or a pharmaceutically acceptable salt, and/or solvate thereof. In embodiment 15, provided is a compound of any one of embodiments A, B, and 1-7, wherein R² is unsubstituted 5-membered heteroaryl (in some embodiments, pyrazolyl); or a stereoisomer, and/or a pharmaceutically acceptable salt, and/or solvate thereof.

Embodiment 16. In embodiment 16, provided is a compound of any one of embodiments A and 1-7, or a stereoisomer, and/or a pharmaceutically acceptable salt, and/or solvate thereof, wherein R² is H, methyl, ethyl, methoxy, —NH-heteroaryl (in some embodiments, —NH-pyrimidinyl), or unsubstituted heteroaryl (in some embodiments, pyrazolyl).

Embodiment 17. In embodiment 17, provided is a compound of any one of embodiments A, B, and 1-7, wherein R² is H, methyl, ethyl, or unsubstituted pyrazolyl; or a stereoisomer, and/or a pharmaceutically acceptable salt, and/or solvate thereof.

Embodiment 18. In embodiment 18, provided is a compound of any one of embodiments A, B, and 1-17, wherein R³ is H; or a stereoisomer, and/or a pharmaceutically acceptable salt, and/or solvate thereof.

Embodiment 19. In embodiment 19, provided is a compound of any one of embodiments 1-17, wherein R³ is F; or a stereoisomer, and/or a pharmaceutically acceptable salt, and/or solvate thereof.

Embodiment 20. In embodiment 20, provided is a compound of any one of embodiments 1-17, wherein R³ is methyl or ethyl; or a stereoisomer, and/or a pharmaceutically acceptable salt, and/or solvate thereof. In embodiment 20, provided is a compound of any one of embodiments A, B, 1, and 2-17, wherein R³ is methyl; or a stereoisomer, and/or a pharmaceutically acceptable salt, and/or solvate thereof.

Embodiment 21. In embodiment 21, provided is a compound of any one of embodiments A, B, and 1, 2, and 4-20, wherein bond b is a double bond and R⁸ is H; or a stereoisomer, and/or a pharmaceutically acceptable salt, and/or solvate thereof.

Embodiment 22. In embodiment 22, provided is a compound of any one of embodiments A, B, and 1, 2, and 4-20, wherein bond b is a double bond and R⁸ is F; or a stereoisomer, and/or a pharmaceutically acceptable salt, and/or solvate thereof.

Embodiment 23. In embodiment 23, provided is a compound of any one of embodiments 1, 2, and 4-20, wherein bond b is a double bond and R⁸ is CN; or a stereoisomer, and/or a pharmaceutically acceptable salt, and/or solvate thereof.

Embodiment 24. In embodiment 24, provided is a compound of any one of embodiments 1, 2, and 4-20, wherein bond b is a double bond and R⁸ is Me; or a stereoisomer, and/or a pharmaceutically acceptable salt, and/or solvate thereof.

Embodiment 25. In embodiment 25, provided is a compound of any one of embodiments A, B, and 1-24, wherein bond a, X¹, X², R⁴, R⁵, R⁶, A, and Ar are according to i); and/or a stereoisomer, and/or a pharmaceutically acceptable salt, and/or solvate thereof.

Embodiment 26a. In embodiment 26a, provided is a compound of embodiment 25 (as it depends from embodiments A, 1, and 2-24), or a stereoisomer, and/or a pharmaceutically acceptable salt, and/or solvate thereof, wherein X¹ is absent, CH₂, CH(OH), or C(CH₃)₂.

Embodiment 26. In embodiment 26, provided is a compound of embodiment 25, wherein X¹ is absent; or a stereoisomer, and/or a pharmaceutically acceptable salt, and/or solvate thereof.

Embodiment 27. In embodiment 27, provided is a compound of embodiment 25, wherein X¹ is CH₂; or a stereoisomer, and/or a pharmaceutically acceptable salt, and/or solvate thereof.

Embodiment 28. In embodiment 28, provided is a compound of any one of embodiments A, B, 1, and 1A, according to the following formula:

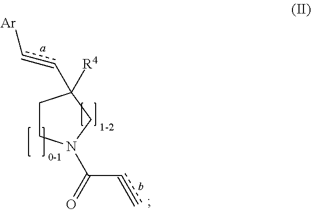

or a stereoisomer, and/or a pharmaceutically acceptable salt, and/or solvate thereof.

Embodiment 29. In embodiment 29, provided is a compound of any one of embodiments 25-28, wherein R⁴ is H; or a stereoisomer, and/or a pharmaceutically acceptable salt, and/or solvate thereof.

Embodiment 30. In embodiment 30, provided is a compound of any one of embodiments 25-28, wherein R⁴ is C₁-C₄ alkyl (methyl); or a stereoisomer, and/or a pharmaceutically acceptable salt, and/or solvate thereof.

Embodiment 31. In embodiment 31, provided is a compound of any one of embodiments 25-28, wherein R⁴ is cyclopropyl; or a stereoisomer, and/or a pharmaceutically acceptable salt, and/or solvate thereof.

Embodiment 32. In embodiment 32, provided is a compound of any one of embodiments 25-28, wherein R⁴ is F; or a stereoisomer, and/or a pharmaceutically acceptable salt, and/or solvate thereof.

Embodiment 33. In embodiment 33, provided is a compound of any one of embodiments 25-28, wherein R⁴ is Cl; or a stereoisomer, and/or a pharmaceutically acceptable salt, and/or solvate thereof.

Embodiment 34. In embodiment 34, provided is a compound of any one of embodiments 25-28, wherein R⁴ is —OH; or a stereoisomer, and/or a pharmaceutically acceptable salt, and/or solvate thereof.

Embodiment 35. In embodiment 35, provided is a compound of any one of embodiments 25-28, wherein R⁴ is —OMe; or a stereoisomer, and/or a pharmaceutically acceptable salt, and/or solvate thereof.

Embodiment 36. In embodiment 36, provided is a compound of any one of embodiments 25-28, wherein R⁴ is CN; or a stereoisomer, and/or a pharmaceutically acceptable salt, and/or solvate thereof.

Embodiment 37. In embodiment 37, provided is a compound of any one of embodiments 25-28, wherein R⁴ is NHR⁷, and R⁷ is selected from the group consisting of H and C₁-C₃ alkyl; or a stereoisomer, and/or a pharmaceutically acceptable salt, and/or solvate thereof.

Embodiment 38. In embodiment 38, provided is a compound of embodiment 25, wherein X¹ and R⁴ together with the carbon to which they are attached form a C₄-C₆ cycloalkylene; or a stereoisomer, and/or a pharmaceutically acceptable salt, and/or solvate thereof.

Embodiment 39. In embodiment 39, provided is a compound of embodiment 25 or 38, wherein X¹ and R⁴ together with the carbon to which they are attached form a C₄ cycloalkylene or C₆ cycloalkylene; or a stereoisomer, and/or a pharmaceutically acceptable salt, and/or solvate thereof.

Embodiment 40. In embodiment 40, provided is a compound of any one of embodiments 25-39, wherein bond a is a double bond; or a stereoisomer, and/or a pharmaceutically acceptable salt, and/or solvate thereof.

Embodiment 41. In embodiment 41, provided is a compound of any one of embodiments 25-40, wherein bond a is a double bond and R⁵ and R⁶ are each H; or a stereoisomer, and/or a pharmaceutically acceptable salt, and/or solvate thereof.

Embodiment 42. In embodiment 42, provided is a compound of any one of embodiments 25-41, wherein bond a is a double bond and is cis; or a stereoisomer, and/or a pharmaceutically acceptable salt, and/or solvate thereof.

Embodiment 43. In embodiment 43, provided is a compound of any one of embodiments 25-41, wherein bond a is a double bond and is trans; or a stereoisomer, and/or a pharmaceutically acceptable salt, and/or solvate thereof.

Embodiment 44. In embodiment 44, provided is a compound of any one of embodiments 25-39, wherein bond a is a triple bond and R⁵ and R⁶ are absent; or a stereoisomer, and/or a pharmaceutically acceptable salt, and/or solvate thereof.

Embodiment 45. In embodiment 45, provided is a compound of any one of embodiments A, B, and 1-24, wherein bond a, X¹, X², R⁴, R⁵, R⁶, A, and Ar are according to ii).

Embodiment 46. In embodiment 46, provided is a compound of embodiment 45, wherein m is 1; or a stereoisomer, and/or a pharmaceutically acceptable salt, and/or solvate thereof.

Embodiment 47. In embodiment 47, provided is a compound of embodiment 45, wherein m is 2; or a stereoisomer, and/or a pharmaceutically acceptable salt, and/or solvate thereof.

Embodiment 48. In embodiment 48, provided is a compound of any one of embodiments 45-47, wherein X¹ is absent; or a stereoisomer, and/or a pharmaceutically acceptable salt, and/or solvate thereof.

Embodiment 48a. In embodiment 48a, provided is a compound of embodiment 45 (as it depends from any one of embodiments A, 1, and 2-24), or a stereoisomer, and/or a pharmaceutically acceptable salt, and/or solvate thereof, wherein m is 0 and X¹ is other than absent.

Embodiment 48b. In embodiment 48b, provided is a compound of any one of embodiments 45-47 (as each of these depends from any one of A, 1, and 2-24), wherein X¹ is absent or is selected from the group consisting of CH₂, O, CH₂O, and OCH₂.

Embodiment 48c. In embodiment 48c, provided is a compound of any one of embodiments 45-47, wherein X¹ is CH₂, CH₂CH₂, or CH(CH₃), preferably CH₂; or a stereoisomer, and/or a pharmaceutically acceptable salt, and/or solvate thereof. In a sub-embodiment of embodiment 48c, provided is a compound wherein X¹ is CH₂.

Embodiment 49. In embodiment 49, provided is a compound of any one of embodiments 45-48c, wherein R⁵ is H; or a stereoisomer, and/or a pharmaceutically acceptable salt, and/or solvate thereof.

Embodiment 50. In embodiment 50, provided is a compound of any one of embodiments 25-49, wherein A is absent; or a stereoisomer, and/or a pharmaceutically acceptable salt, and/or solvate thereof.

Embodiment 51a. In embodiment 51a, provided is a compound of any one of embodiments 25-44 (as each of these depends from any one of embodiments A, 1, and 2-24), or a stereoisomer, and/or a pharmaceutically acceptable salt, and/or solvate thereof, wherein A is selected from the group consisting of CH₂, CH(OH), CH(OCH₃), and C(CH₃)₂.

Embodiment 51. In embodiment 51, provided is a compound of any one of embodiments 25-50, wherein Ar is aryl optionally substituted with 1, 2, 3, or 4 R⁹ groups; or a stereoisomer, and/or a pharmaceutically acceptable salt, and/or solvate thereof.

Embodiment 52a. In embodiment 52a, provided is a compound of any one of embodiments 20-46, or a stereoisomer, and/or a pharmaceutically acceptable salt, and/or solvate thereof, wherein Ar is phenyl optionally substituted with 1, 2, 3, or 4 R⁹ groups (in some embodiments, the phenyl is optionally substituted with 1 R⁹).

Embodiment 52. In embodiment 52, provided is a compound of any one of embodiments 25-51, wherein Ar is phenyl optionally substituted with 1, 2, 3, or 4 R⁹ groups; or a stereoisomer, and/or a pharmaceutically acceptable salt, and/or solvate thereof.

Embodiment 53a. In embodiment 53a, provided is a compound of any one of embodiments 20-46, or a stereoisomer, and/or a pharmaceutically acceptable salt, and/or solvate thereof, wherein Ar is heteroaryl, (preferably oxazolyl, imidazolyl, or pyridinyl), optionally substituted with 1, 2, 3, or 4 R⁹ groups (in some embodiments, the heteroaryl is optionally substituted with 1 R⁹).

Embodiment 53. In embodiment 53, provided is a compound of any one of embodiments 25-50, wherein Ar is heteroaryl group, preferably oxazolyl, imidazolyl, or pyridinyl, optionally substituted with 1, 2, 3, or 4 R⁹ groups; or a stereoisomer, and/or a pharmaceutically acceptable salt, and/or solvate thereof.

Embodiment 54. In embodiment 54, provided is a compound of any one of embodiments 1-24, wherein bond a, X¹, X², R⁴, R⁵, R⁶, A, and Ar are according to iii).

Embodiment 55. In embodiment 55, provided is a compound of embodiment 54, wherein X¹ is absent, and R⁴ is H; or a stereoisomer, and/or a pharmaceutically acceptable salt, and/or solvate thereof.

Embodiment 56. In embodiment 56, provided is a compound of any one of embodiments 1-24, wherein bond a, X¹, X², R⁴, R⁵, R⁶, A, and Ar are according to iv).

Embodiment 57. In embodiment 57, provided is a compound of embodiment 56, wherein X² is CH; or a stereoisomer, and/or a pharmaceutically acceptable salt, and/or solvate thereof.

Embodiment 58. In embodiment 58, provided is a compound of embodiment 56, wherein X² is N; or a stereoisomer, and/or a pharmaceutically acceptable salt, and/or solvate thereof.

Embodiment 59. In embodiment 59, provided is a compound of any one of embodiments 56-58, wherein X¹ is absent and R⁴ is H; or a stereoisomer, and/or a pharmaceutically acceptable salt, and/or solvate thereof.

Embodiment 60. In embodiment 60, provided is a compound of any one of embodiments 56-59, wherein X³ is O, NH, N(Me); or a stereoisomer, and/or a pharmaceutically acceptable salt, and/or solvate thereof.

Embodiment 61. In embodiment 61, provided is a compound of any one of embodiments 56-60, wherein Ar₁ is benzo or pyrido, optionally substituted with 1, 2, 3, or 4 R⁹ groups; or a stereoisomer, and/or a pharmaceutically acceptable salt, and/or solvate thereof.

Embodiment 62. In embodiment 62, provided is a compound of any one of embodiments 1-61, wherein each R⁹ is independently selected from the group consisting of C₁-C₆ alkyl, C₂-C₆ alkynyl, halo, C₁-C₆ haloalkyl, and C₁-C₆ alkoxy; or each R⁹ is independently selected from the group consisting of methyl, ethynyl, F, CF₃, and methoxy; or a stereoisomer, and/or a pharmaceutically acceptable salt, and/or solvate thereof.

Embodiment 63. In embodiment 63, provided is a compound of any one of embodiments 1-62, wherein 1 or 2 R⁹ are present and are independently selected; or a stereoisomer, and/or a pharmaceutically acceptable salt, and/or solvate thereof.

Embodiment 64. In embodiment 64, provided is a compound of any one of embodiments 1-63, wherein one R⁹ is CF₃; or a stereoisomer, and/or a pharmaceutically acceptable salt, and/or solvate thereof.

Embodiment 65a. In embodiment 65a, provided is a compound of any one of embodiments 1-64 wherein

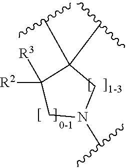

is

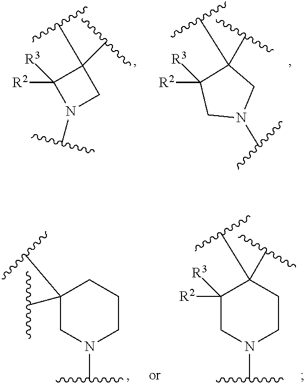

or a stereoisomer, and/or a pharmaceutically acceptable salt, and/or solvate thereof.

Embodiment 65. In embodiment 65, provided is a compound of any one of embodiments 1-64, wherein

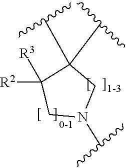

is

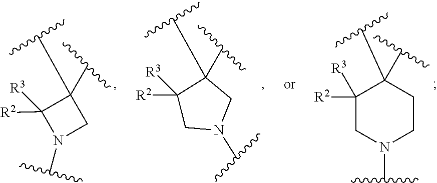

or a stereoisomer, and/or a pharmaceutically acceptable salt, and/or solvate thereof.

Embodiment 66. In embodiment 66, provided is a compound of any one of embodiments 1-65, wherein

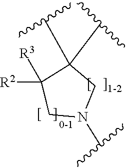

is

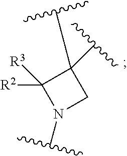

or a stereoisomer, and/or a pharmaceutically acceptable salt, and/or solvate thereof.

Embodiment 67. In embodiment 67, provided is a compound of any one of embodiments 1-65, wherein

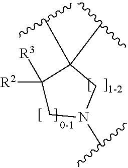

is

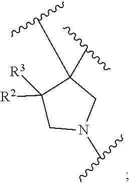

or a stereoisomer, and/or a pharmaceutically acceptable salt, and/or solvate thereof.

Embodiment 68. In embodiment 68, provided is a compound of any one of embodiments 1-65, wherein

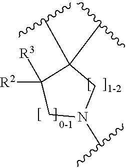

is

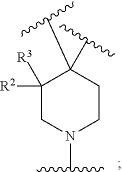

or a stereoisomer, and/or a pharmaceutically acceptable salt, and/or solvate thereof.

Embodiment 69. In embodiment 69, provided is a compound of embodiment 1 being selected from the compounds in Table 1; or a stereoisomer, and/or a pharmaceutically acceptable salt, and/or solvate thereof.

Embodiment 70. In embodiment 70, provided is a compound of embodiment 1, selected from the group consisting of:

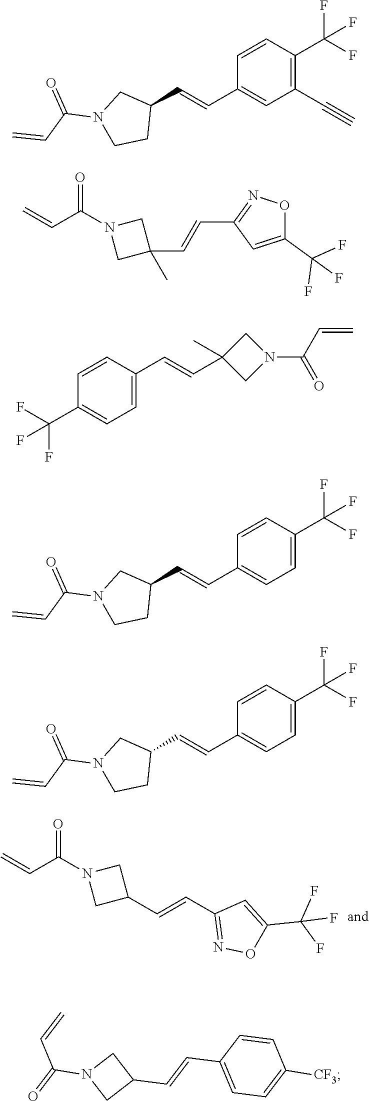

or a stereoisomer, and/or a pharmaceutically acceptable salt, and/or solvate thereof.

Embodiment 71. In embodiment 71, provided is a pharmaceutical composition, comprising a pharmaceutically effective amount of the compound according to any one of embodiments 1-106 or a stereoisomer, and/or a pharmaceutically acceptable salt, and/or solvate thereof; and a pharmaceutically acceptable carrier.

Embodiment 72. In embodiment 72, provided is a method of treating cancer in a subject in need thereof, comprising administering to the subject a therapeutically effective amount of the compound of any one of embodiments 1-106, or a stereoisomer, and/or a pharmaceutically acceptable salt, and/or solvate thereof, or the pharmaceutical composition of embodiment 71.

Embodiment 73. In embodiment 73, provided is a method of embodiment 72, wherein the cancer is selected from the group consisting of bladder cancer, breast cancer, ovarian cancer, pancreatic ductal adenocarcinoma (PDAC), glioblastoma, gastric cancer, cervical cancer, colon cancer, endometrial cancer, head and neck cancer, lung cancer, melanoma, multiple myeloma, leukemia, non-Hodgkin's lymphoma, prostate cancer, rectal cancer, malignant melanomas, alimentary/gastrointestinal tract cancer, liver cancer, skin cancer, lymphoma, malignant pleural mesothelioma (MPM), kidney cancer, muscle cancer, bone cancer, brain cancer, eye or ocular cancer, rectal cancer, colorectal cancer, cervical cancer, oral cancer, benign and malignant tumors, stomach cancer, corpus uteri, testicular cancer, renal cancer, throat cancer, acute lymphocytic leukemia, acute myelogenous leukemia, Ewing's Sarcoma, Kaposi's Sarcoma, basal cell carcinoma and squamous cell carcinoma, small cell lung cancer, choriocarcinoma, rhabdomyosarcoma, angiosarcoma, hemangioendothelioma, Wilms Tumor, neuroblastoma, mouth/pharynx cancer, esophageal cancer, larynx cancer, neurofibromatosis, tuberous sclerosis, hemangiomas, and lymphangiogenesis.

Embodiment 74. In embodiment 74, provided is a method of embodiment 72, wherein the cancer is selected from the group consisting of glioblastoma, gastric cancer, colorectal cancer, pancreatic ductal adenocarcinoma (PDAC), and malignant pleural mesothelioma (MPM).

Embodiment 75. In embodiment 75, provided is a method of any one of embodiments 72-74, wherein the method further comprises simultaneously or sequentially administering one or more KRAS G12C and/or G12D inhibitors; one or more CDK4/6 inhibitors; one or more EGFR inhibitors; one or more RAF inhibitors; one or more MEK inhibitors; one or more Wnt signaling inhibitors, such as anti-β-catenin inhibitors, GSK3 inhibitors. JNK inhibitors, and CK1 inhibitors; one or more TGF-β signaling inhibitors, such as atezolizumab, durvalumab, and avelumab; one or more PD-1/PD-L1 inhibitors; and/or radiotherapy.

Embodiment 75. In embodiment 75, provided is a method of any one of embodiments 72-74, wherein the method further comprises simultaneously or sequentially administering one or more additional anti-cancer therapies, preferably wherein the one or more additional therapies comprises a KRAS G12C and/or G12D inhibitor, more preferably, wherein KRAS G12C and/or G12D inhibitor is sotorasib or adagrasib.

Embodiment 76. In embodiment 76, provided is a method of inhibiting TEAD1, 2, 3, and/or 4 in a subject or in a sample, comprising administering to the subject a therapeutically effective amount of the compound of any one of embodiments 1-106, or a stereoisomer, and/or a pharmaceutically acceptable salt, and/or solvate thereof; or the pharmaceutical composition of embodiment 71.

Embodiment 77. In embodiment 77, provided is a method of embodiment 76, wherein TEAD1 is inhibited selectively over TEAD2, 3, and 4.

Table 1 shows the structures of compounds described in the Examples of this disclosure.

TABLE 1
TEAD Inhibitor Compounds

| Cmpd | Structure | Cmpd | Structure |
|---|---|---|---|
| 1 | 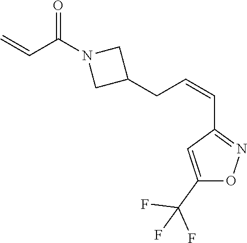 | 2 | 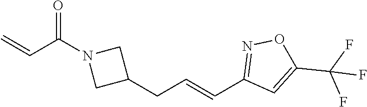 [SMILES: C=CC(=O)N1CC(C/C=C/c2cc(C(F)(F)F)on2)C1] |
| 3 | 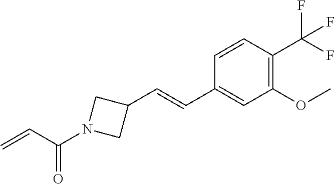 [SMILES: C=CC(=O)N1CC(/C=C/c2ccc(C(F)(F)F)c(OC)c2)C1] | 4 | 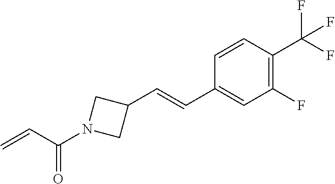 [SMILES: C=CC(=O)N1CC(/C=C/c2ccc(C(F)(F)F)c(F)c2)C1] |
| 5 | 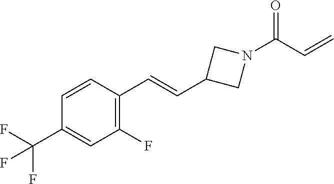 [SMILES: C=CC(=O)N1CC(/C=C/c2ccc(C(F)(F)F)cc2F)C1] | 6 | 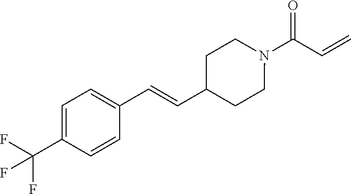 [SMILES: C=CC(=O)N1CCC(/C=C/c2ccc(C(F)(F)F)cc2)CC1] |
| 7 | 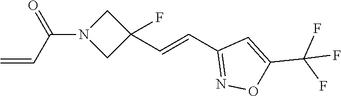 [SMILES: C=CC(=O)N1CC(F)(/C=C/c2cc(C(F)(F)F)on2)C1] | 8 | 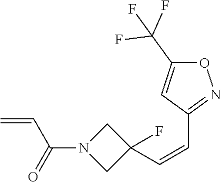 [SMILES: C=CC(=O)N1CC(F)(/C=C\c2cc(C(F)(F)F)on2)C1] |
| 9 | 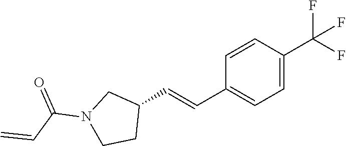 [SMILES: C=CC(=O)N1CC[C@@H](/C=C/c2ccc(C(F)(F)F)cc2)C1] | 10 | 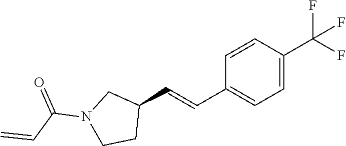 [SMILES: C=CC(=O)N1CC[C@H](/C=C/c2ccc(C(F)(F)F)cc2)C1] |
| 11 | 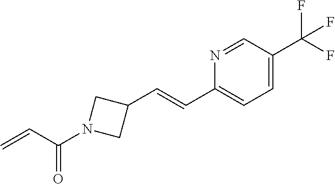 [SMILES: C=CC(=O)N1CC(/C=C/c2ccc(C(F)(F)F)cn2)C1] | 12 | 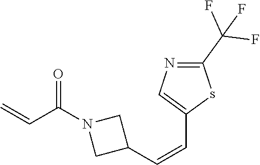 |
| 13 | 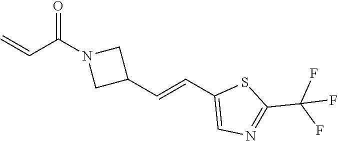 [SMILES: C=CC(=O)N1CC(/C=C/c2cnc(C(F)(F)F)s2)C1] | 14 | 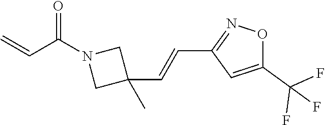 [SMILES: C=CC(=O)N1CC(C)(/C=C/c2cc(C(F)(F)F)on2)C1] |
| 15 | 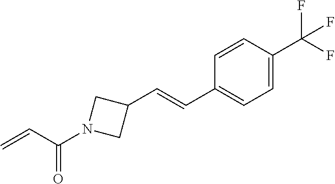 [SMILES: C=CC(=O)N1CC(/C=C/c2ccc(C(F)(F)F)cc2)C1] | 16 | 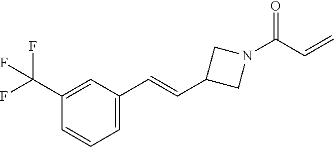 [SMILES: C=CC(=O)N1CC(/C=C/c2cccc(C(F)(F)F)c2)C1] |
| 17 | 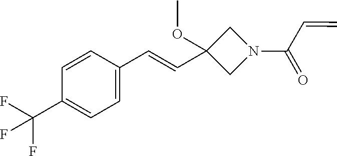 [SMILES: C=CC(=O)N1CC(/C=C/c2ccc(C(F)(F)F)cc2)(OC)C1] | 18 | 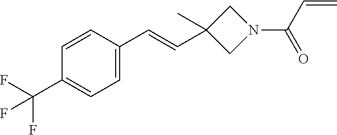 [SMILES: C=CC(=O)N1CC(C)(/C=C/c2ccc(C(F)(F)F)cc2)C1] |
| 19 | 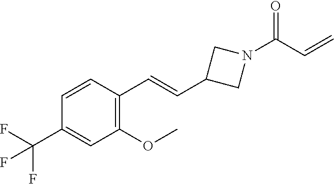 [SMILES: C=CC(=O)N1CC(/C=C/c2ccc(C(F)(F)F)cc2OC)C1] | 20 | 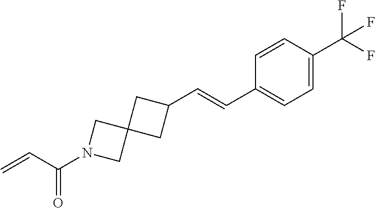 [SMILES: C=CC(=O)N1CC2(CC(/C=C/c3ccc(C(F)(F)F)cc3)C2)C1] |
| 21 | 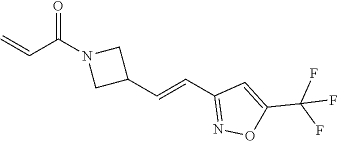 [SMILES: C=CC(=O)N1CC(/C=C/c2cc(C(F)(F)F)on2)C1] | 22 | 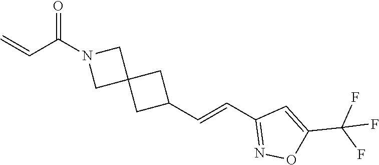 [SMILES: C=CC(=O)N1CC2(CC(/C=C/c3cc(C(F)(F)F)on3)C2)C1] |
| 23 | 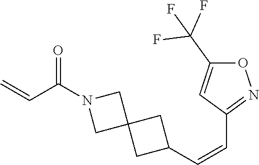 [SMILES: C=CC(=O)N1CC2(CC(/C=C\c3cc(C(F)(F)F)on3)C2)C1] | 24 | 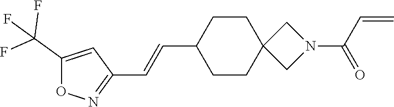 [SMILES: C=CC(=O)N1CC2(CCC(/C=C/c3cc(C(F)(F)F)on3)CC2)C1] |
| 25 | 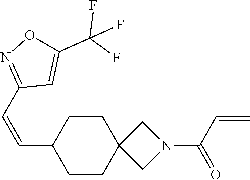 [SMILES: C=CC(=O)N1CC2(CCC(/C=C\c3cc(C(F)(F)F)on3)CC2)C1] | 26 | 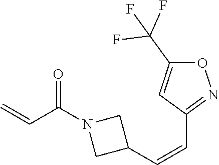 [SMILES: C=CC(=O)N1CC(/C=C\c2cc(C(F)(F)F)on2)C1] |
| 27 | 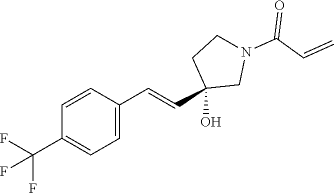 [SMILES: C=CC(=O)N1CC[C@](O)(/C=C/c2ccc(C(F)(F)F)cc2)C1] | 28 | 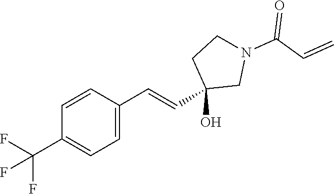 [SMILES: C=CC(=O)N1CC[C@@](O)(/C=C/c2ccc(C(F)(F)F)cc2)C1] |
| 29 | 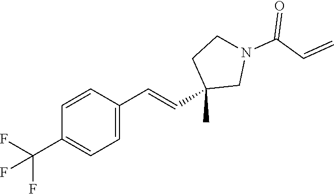 [SMILES: C=CC(=O)N1CC[C@@](C)(/C=C/c2ccc(C(F)(F)F)cc2)C1] | 30 | 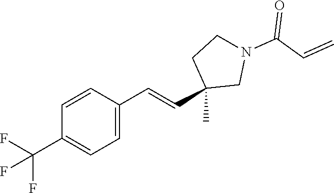 [SMILES: C=CC(=O)N1CC[C@](C)(/C=C/c2ccc(C(F)(F)F)cc2)C1] |
| 31 | 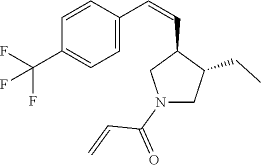 [SMILES: C=CC(=O)N1C[C@@H](CC)[C@H](/C=C\c2ccc(C(F)(F)F)cc2)C1] | 32 | 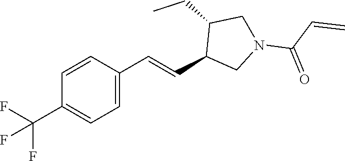 [SMILES: C=CC(=O)N1C[C@@H](CC)[C@H](/C=C/c2ccc(C(F)(F)F)cc2)C1] |
| 33 | 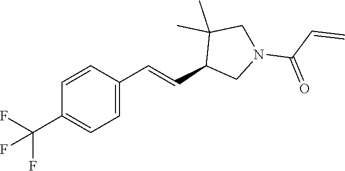 [SMILES: C=CC(=O)N1C[C@@H](/C=C/c2ccc(C(F)(F)F)cc2)C(C)(C)C1] | 34 | 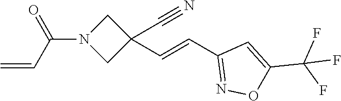 [SMILES: C=CC(=O)N1CC(C#N)(/C=C/c2cc(C(F)(F)F)on2)C1] |
| 35 | 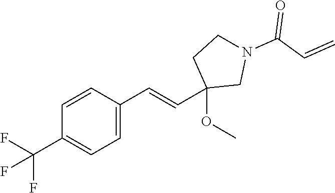 [SMILES: C=CC(=O)N1CCC(/C=C/c2ccc(C(F)(F)F)cc2)(OC)C1] | 36 | 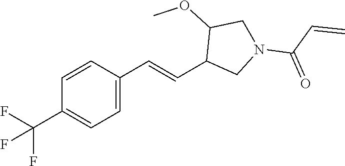 [SMILES: C=CC(=O)N1CC(/C=C/c2ccc(C(F)(F)F)cc2)C(OC)C1] |
| 37 | 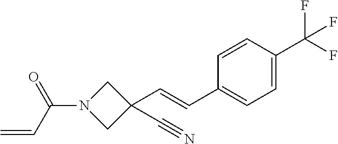 [SMILES: C=CC(=O)N1CC(C#N)(/C=C/c2ccc(C(F)(F)F)cc2)C1] | 38 | 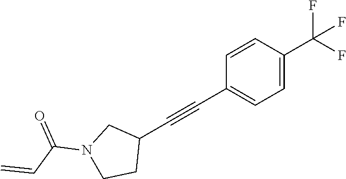 [SMILES: C=CC(=O)N1CCC(C#Cc2ccc(C(F)(F)F)cc2)C1] |
| 39 | 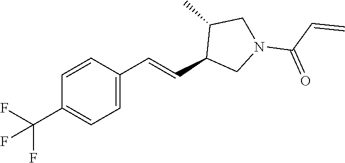 [SMILES: C=CC(=O)N1C[C@@H](C)[C@H](/C=C/c2ccc(C(F)(F)F)cc2)C1] | 40 | 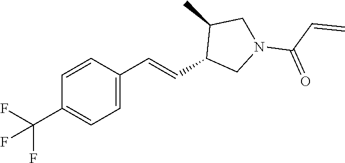 [SMILES: C=CC(=O)N1C[C@H](C)[C@@H](/C=C/c2ccc(C(F)(F)F)cc2)C1] |
| 41 | 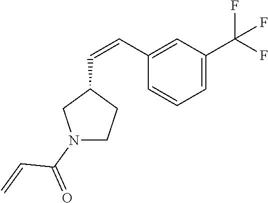 [SMILES: C=CC(=O)N1CC[C@@H](/C=C\c2cccc(C(F)(F)F)c2)C1] | 42 | 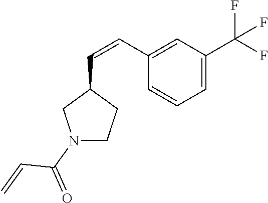 [SMILES: C=CC(=O)N1CC[C@H](/C=C\c2cccc(C(F)(F)F)c2)C1] |
| 43 | 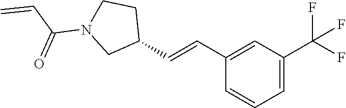 [SMILES: C=CC(=O)N1CC[C@H](/C=C/c2cccc(C(F)(F)F)c2)C1] | 44 | 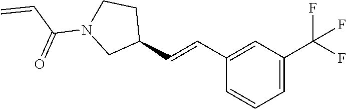 [SMILES: C=CC(=O)N1CC[C@@H](/C=C/c2cccc(C(F)(F)F)c2)C1] |
| 45 | 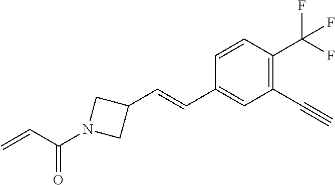 [SMILES: C#Cc1cc(/C=C/C2CN(C(=O)C=C)C2)ccc1C(F)(F)F] | 46 | 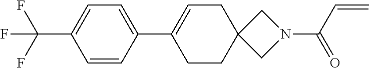 [SMILES: C=CC(=O)N1CC2(CC=C(c3ccc(C(F)(F)F)cc3)CC2)C1] |
| 47 | 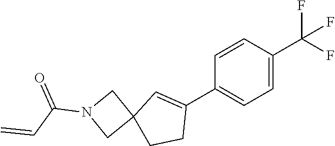 [SMILES: C=CC(=O)N1CC2(C=C(c3ccc(C(F)(F)F)cc3)CC2)C1] | 48 | 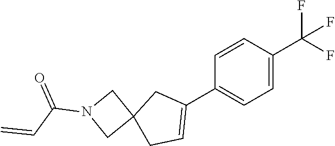 [SMILES: C=CC(=O)N1CC2(CC=C(c3ccc(C(F)(F)F)cc3)C2)C1] |
| 49 | 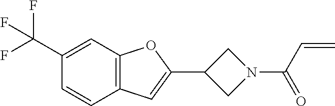 [SMILES: C=CC(=O)N1CC(c2cc3ccc(C(F)(F)F)cc3o2)C1] | 50 | 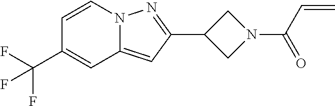 [SMILES: C=CC(=O)N1CC(c2cc3cc(C(F)(F)F)ccn3n2)C1] |
| 51 | 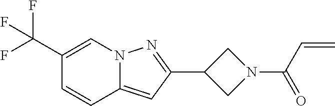 [SMILES: C=CC(=O)N1CC(c2cc3ccc(C(F)(F)F)cn3n2)C1] | 52 | 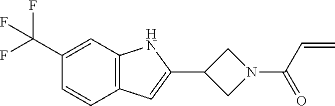 [SMILES: C=CC(=O)N1CC(c2cc3ccc(C(F)(F)F)cc3[nH]2)C1] |
| 53 | 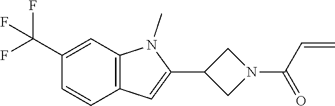 [SMILES: C=CC(=O)N1CC(c2cc3ccc(C(F)(F)F)cc3n2C)C1] | 54 | 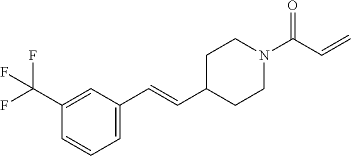 [SMILES: C=CC(=O)N1CCC(/C=C/c2cccc(C(F)(F)F)c2)CC1] |
| 55 | 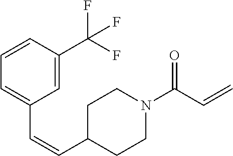 [SMILES: C=CC(=O)N1CCC(/C=C\c2cccc(C(F)(F)F)c2)CC1] | 56 | 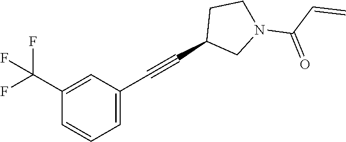 [SMILES: C=CC(=O)N1CC[C@H](C#Cc2cccc(C(F)(F)F)c2)C1] |
| 57 | 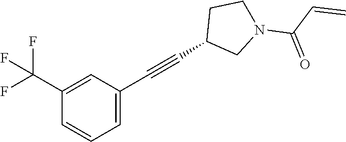 [SMILES: C=CC(=O)N1CC[C@@H](C#Cc2cccc(C(F)(F)F)c2)C1] | 58 | 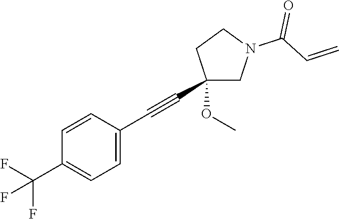 [SMILES: C=CC(=O)N1CC[C@@](C#Cc2ccc(C(F)(F)F)cc2)(OC)C1] |
| 59 | 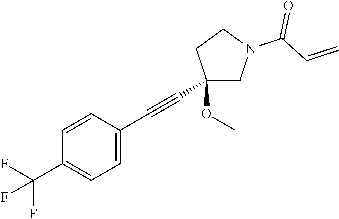 [SMILES: C=CC(=O)N1CC[C@](C#Cc2ccc(C(F)(F)F)cc2)(OC)C1] | 60 | 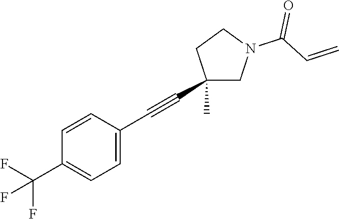 [SMILES: C=CC(=O)N1CC[C@](C)(C#Cc2ccc(C(F)(F)F)cc2)C1] |
| 61 | 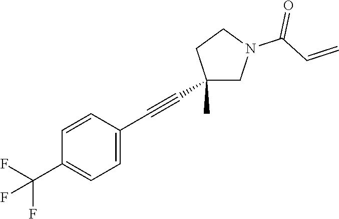 [SMILES: C=CC(=O)N1CC[C@@](C)(C#Cc2ccc(C(F)(F)F)cc2)C1] | 62 | 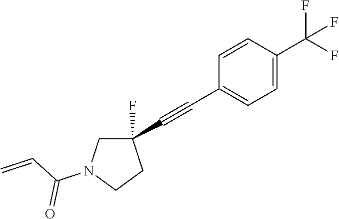 [SMILES: C=CC(=O)N1CC[C@](F)(C#Cc2ccc(C(F)(F)F)cc2)C1] |
| 63 | 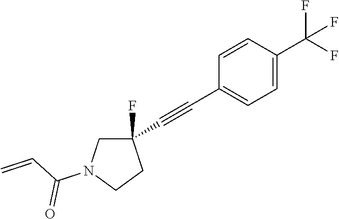 [SMILES: C=CC(=O)N1CC[C@@](F)(C#Cc2ccc(C(F)(F)F)cc2)C1] | 64 | 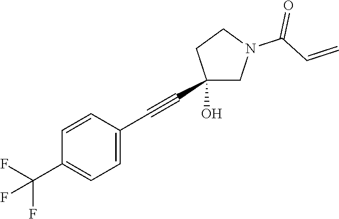 [SMILES: C=CC(=O)N1CC[C@](O)(C#Cc2ccc(C(F)(F)F)cc2)C1] |
| 65 | 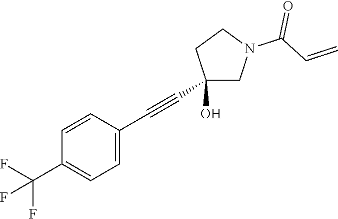 [SMILES: C=CC(=O)N1CC[C@@](O)(C#Cc2ccc(C(F)(F)F)cc2)C1] | 66 | 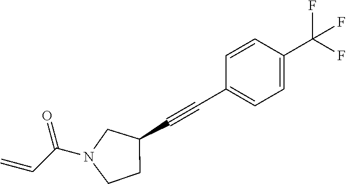 [SMILES: C=CC(=O)N1CC[C@H](C#Cc2ccc(C(F)(F)F)cc2)C1] |
| 67 | 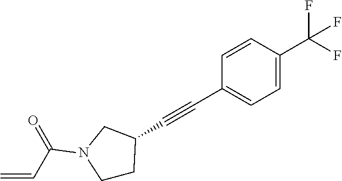 [SMILES: C=CC(=O)N1CC[C@@H](C#Cc2ccc(C(F)(F)F)cc2)C1] | 68 | 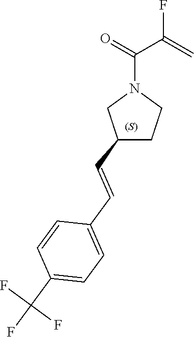 |
| 69 | 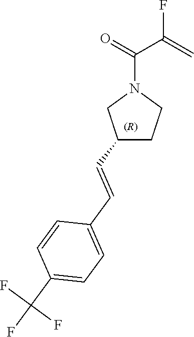 | 70 | 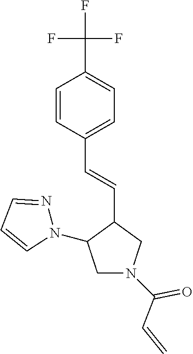 [SMILES: C=CC(=O)N1CC(/C=C/c2ccc(C(F)(F)F)cc2)C(n2cccn2)C1] |
| 71 | 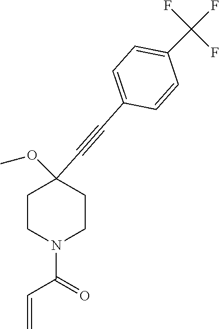 [SMILES: C=CC(=O)N1CCC(C#Cc2ccc(C(F)(F)F)cc2)(OC)CC1] | 72 | 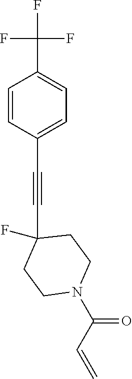 [SMILES: C=CC(=O)N1CCC(F)(C#Cc2ccc(C(F)(F)F)cc2)CC1] |
| 73 | 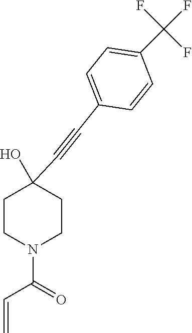 [SMILES: C=CC(=O)N1CCC(O)(C#Cc2ccc(C(F)(F)F)cc2)CC1] | 74 | 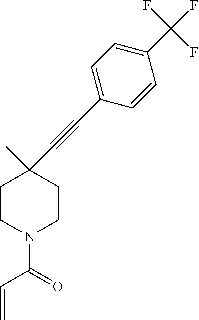 [SMILES: C=CC(=O)N1CCC(C)(C#Cc2ccc(C(F)(F)F)cc2)CC1] |
| 75 | 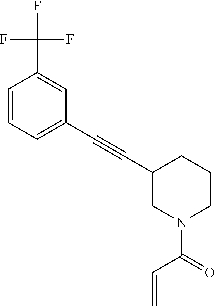 [SMILES: C=CC(=O)N1CCCC(C#Cc2cccc(C(F)(F)F)c2)C1] | 76 | 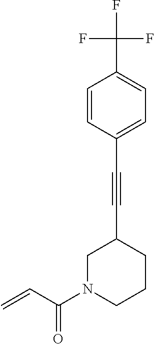 [SMILES: C=CC(=O)N1CCCC(C#Cc2ccc(C(F)(F)F)cc2)C1] |
| 77 | 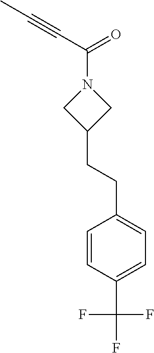 [SMILES: CC#CC(=O)N1CC(CCc2ccc(C(F)(F)F)cc2)C1] | 78 | 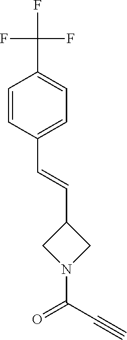 [SMILES: C#CC(=O)N1CC(/C=C/c2ccc(C(F)(F)F)cc2)C1] |
| 79 | 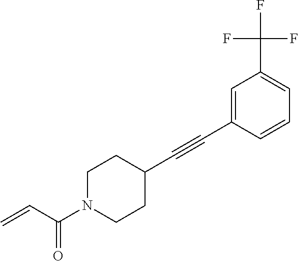 [SMILES: C=CC(=O)N1CCC(C#Cc2cccc(C(F)(F)F)c2)CC1] | 80 | 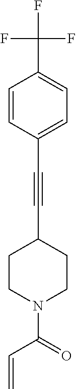 [SMILES: C=CC(=O)N1CCC(C#Cc2ccc(C(F)(F)F)cc2)CC1] |
| 81 | 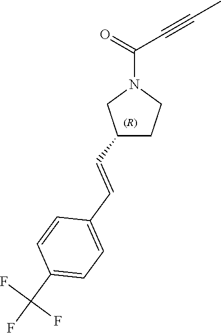 | 82 | 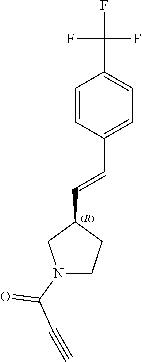 |
| 83 | 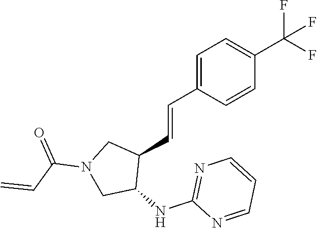 [SMILES: C=CC(=O)N1C[C@@H](/C=C/c2ccc(C(F)(F)F)cc2)[C@H](Nc2ncccn2)C1] | 84 | 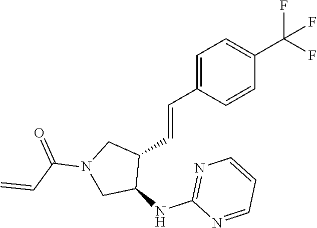 [SMILES: C=CC(=O)N1C[C@H](Nc2ncccn2)[C@@H](/C=C/c2ccc(C(F)(F)F)cc2)C1] |
| 85 | 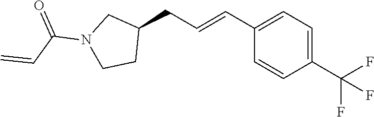 [SMILES: C=CC(=O)N1CC[C@H](C/C=C/c2ccc(C(F)(F)F)cc2)C1] | 86 | 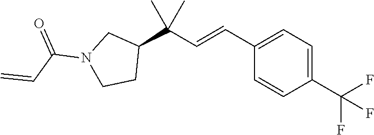 [SMILES: C=CC(=O)N1CC[C@H](C(C)(C)/C=C/c2ccc(C(F)(F)F)cc2)C1] |
| 87 | 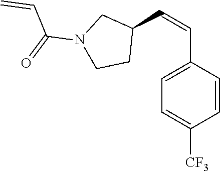 [SMILES: C=CC(=O)N1CC[C@H](/C=C\c2ccc(C(F)(F)F)cc2)C1] | 88 | 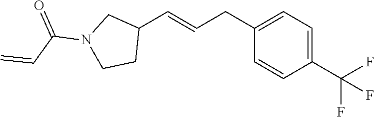 [SMILES: C=CC(=O)N1CCC(/C=C/Cc2ccc(C(F)(F)F)cc2)C1] |
| 89 | 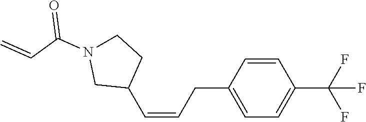 [SMILES: C=CC(=O)N1CCC(/C=C\Cc2ccc(C(F)(F)F)cc2)C1] | 90 | 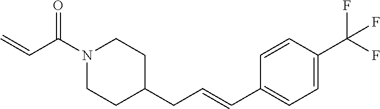 [SMILES: C=CC(=O)N1CCC(C/C=C/c2ccc(C(F)(F)F)cc2)CC1] |
| 91 | 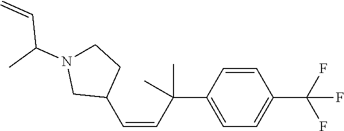 [SMILES: C=CC(C)N1CCC(/C=C\C(C)(C)c2ccc(C(F)(F)F)cc2)C1] | 92 | 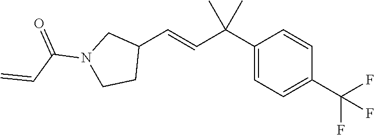 [SMILES: C=CC(=O)N1CCC(/C=C/C(C)(C)c2ccc(C(F)(F)F)cc2)C1] |
| 93 | 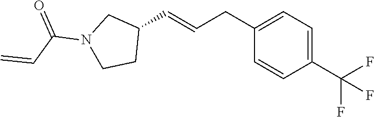 [SMILES: C=CC(=O)N1CC[C@@H](/C=C/Cc2ccc(C(F)(F)F)cc2)C1] | 94 | 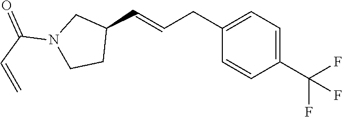 [SMILES: C=CC(=O)N1CC[C@H](/C=C/Cc2ccc(C(F)(F)F)cc2)C1] |
| 95 | 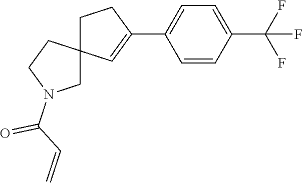 [SMILES: C=CC(=O)N1CCC2(C=C(c3ccc(C(F)(F)F)cc3)CC2)C1] | 96 | 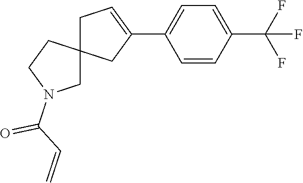 [SMILES: C=CC(=O)N1CCC2(CC=C(c3ccc(C(F)(F)F)cc3)C2)C1] |
| 97 | 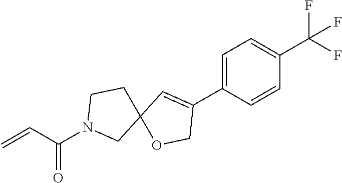 [SMILES: C=CC(=O)N1CCC2(C=C(c3ccc(C(F)(F)F)cc3)CO2)C1] | 98 | 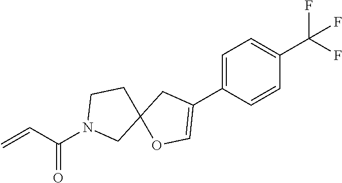 [SMILES: C=CC(=O)N1CCC2(CC(c3ccc(C(F)(F)F)cc3)=CO2)C1] |
| 99 | 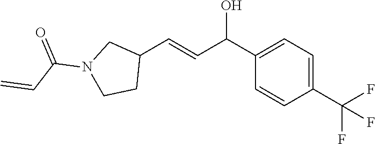 [SMILES: C=CC(=O)N1CCC(/C=C/C(O)c2ccc(C(F)(F)F)cc2)C1] | 100 | 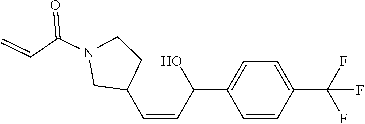 [SMILES: C=CC(=O)N1CCC(/C=C\C(O)c2ccc(C(F)(F)F)cc2)C1] |
| 101 | 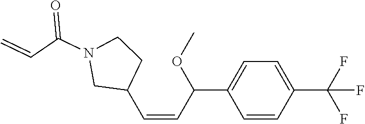 [SMILES: C=CC(=O)N1CCC(/C=C\C(OC)c2ccc(C(F)(F)F)cc2)C1] | 102 | 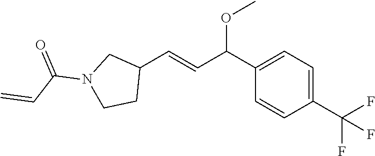 [SMILES: C=CC(=O)N1CCC(/C=C/C(OC)c2ccc(C(F)(F)F)cc2)C1] |
| 103 | 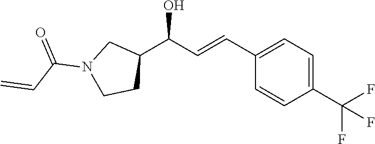 [SMILES: C=CC(=O)N1CC[C@H]([C@@H](O)/C=C/c2ccc(C(F)(F)F)cc2)C1] | 104 | 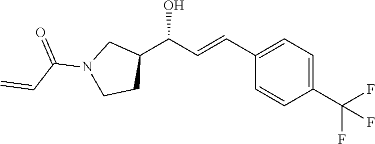 [SMILES: C=CC(=O)N1CC[C@H]([C@H](O)/C=C/c2ccc(C(F)(F)F)cc2)C1] |
| 105 | 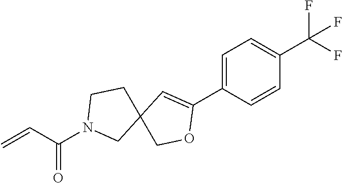 [SMILES: C=CC(=O)N1CCC2(C=C(c3ccc(C(F)(F)F)cc3)OC2)C1] | 106 | 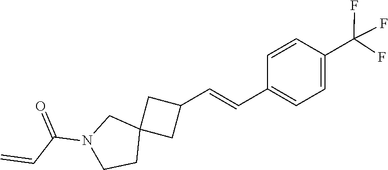 [SMILES: C=CC(=O)N1CCC2(CC(/C=C/c3ccc(C(F)(F)F)cc3)C2)C1] |

In some or any embodiments, provided herein are:
(a) compounds as described herein, e.g., of Formula (I), (II), any embodiments herein, and any of Compounds 1-106, and pharmaceutically acceptable salts and compositions thereof;
(b) compounds as described herein, e.g., of Formula (I), (II), any embodiments herein, and any of Compounds 1-106, and pharmaceutically acceptable salts and compositions thereof for use in the treatment of cancer and/or conditions modulated by TEAD1, 2, 3, and/or 4;
(c) processes for the preparation of compounds as described herein, e.g., of Formula (I), (II), any embodiments herein, and any of Compounds 1-106, as described in more detail elsewhere herein;
(d) pharmaceutical formulations comprising a compound as described herein, e.g., of Formula (I), (II), any embodiments herein, and any of Compounds 1-106, or a pharmaceutically acceptable salt thereof together with a pharmaceutically acceptable carrier or diluent;
(e) a method for the treatment of a condition associated with TEAD1, 2, 3, and/or 4 function in a subject that includes the administration of a therapeutically or prophylactically effective amount of a compound as described herein, e.g., of Formula (I), (II), any embodiments herein, and any of Compounds 1-106, its pharmaceutically acceptable salt or composition;
(f) a method for the treatment of cancer in a subject that includes the administration of a therapeutically or prophylactically effective amount of a compound as described herein, e.g., of Formula (I), (II), any embodiments herein, and any of Compounds 1-106, its pharmaceutically acceptable salt or composition;
(g) pharmaceutical formulations comprising a compound as described herein, e.g., of Formula (I), (II), any embodiments herein, and any of Compounds 1-106, or a pharmaceutically acceptable salt thereof together with one or more other effective agents for treating cancer and/or conditions modulated by TEAD1, 2, 3, and/or 4, optionally in a pharmaceutically acceptable carrier or diluent;
(h) a method for the treatment of cancer in a subject that includes the administration of a therapeutically or prophylactically effective amount of a compound as described herein, e.g., of Formula (I), (II), any embodiments herein, and any of Compounds 1-106, its pharmaceutically acceptable salt or composition in combination and/or alternation with one or more agent for the treatment of cancer and/or conditions modulated by TEAD1, 2, 3, and/or 4; and
(i) a method for the treatment of a condition associated with TEAD1, 2, 3, and/or 4 function in a subject that includes the administration of a therapeutically or prophylactically effective amount of a compound as described herein, e.g., of Formula (I), (II), any embodiments herein, and any of Compounds 1-106, its pharmaceutically acceptable salt or composition in combination and/or alternation with one or more agent for the treatment of cancer.
(j) use of any compound described herein, e.g., of Formula (I), (II), any embodiments herein, and any of Compounds 1-106, or a composition comprising any compound described herein, e.g., of Formula (I), (II), any embodiments herein, and any of Compounds 1-106, for the treatment of a condition associated with TEAD1, 2, 3, and/or 4 function described herein (e.g., cancer), optionally in combination and/or alternation with one or more agent for the treatment of cancer.

### Isomers and Optically Active Compounds

It is appreciated that compounds provided herein have several chiral centers and may exist in and be isolated in optically active and racemic forms. It is to be understood that any racemic, optically-active, diastereomeric, geometric isomeric, tautomeric, or other stereoisomeric forms, mixture, or combination thereof, of a compound provided herein, which possess the useful properties described herein is within the scope of the invention. It being well known in the art how to prepare optically active forms (in some or any embodiments, by resolution of the racemic form by recrystallization techniques, by synthesis from optically-active starting materials, by chiral synthesis, or by chromatographic separation using a chiral stationary phase).

In some or any embodiments, methods to obtain optically active materials (in some embodiments, compounds which are substantially pure or are “substantially free of” of an undesignated isomer or are “substantially in the absence of” of an undesignated isomer) are known in the art, and include at least the following.
i) physical separation of crystals—a technique whereby macroscopic crystals of the individual stereoisomers are manually separated. This technique can be used if crystals of the separate stereoisomers exist, i.e., the material is a conglomerate, and the crystals are visually distinct;
ii) simultaneous crystallization—a technique whereby the individual stereoisomers are separately crystallized from a solution of the racemate, possible only if the latter is a conglomerate in the solid state;
iii) enzymatic resolutions—a technique whereby partial or complete separation of a racemate by virtue of differing rates of reaction for the stereoisomers with an enzyme;
iv) enzymatic asymmetric synthesis—a synthetic technique whereby at least one step of the synthesis uses an enzymatic reaction to obtain a stereoisomerically pure or enriched synthetic precursor of the desired stereoisomer;
v) chemical asymmetric synthesis—a synthetic technique whereby the desired stereoisomer is synthesized from an achiral precursor under conditions that produce asymmetry (i.e., chirality) in the product, which may be achieved using chiral catalysts or chiral auxiliaries;
vi) diastereomer separations—a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;
vii) first- and second-order asymmetric transformations—a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer;
viii) kinetic resolutions—this technique refers to the achievement of partial or complete resolution of a racemate (or of a further resolution of a partially resolved compound) by virtue of unequal reaction rates of the stereoisomers with a chiral, non-racemic reagent or catalyst under kinetic conditions;
ix) stereospecific synthesis from non-racemic precursors—a synthetic technique whereby the desired stereoisomer is obtained from non-chiral starting materials and where the stereochemical integrity is not or is only minimally compromised over the course of the synthesis;
x) chiral liquid chromatography—a technique whereby the stereoisomers of a racemate are separated in a liquid mobile phase by virtue of their differing interactions with a stationary phase. The stationary phase can be made of chiral material or the mobile phase can contain an additional chiral material to provoke the differing interactions;
xi) chiral gas chromatography—a technique whereby the racemate is volatilized and stereoisomers are separated by virtue of their differing interactions in the gaseous mobile phase with a column containing a fixed non-racemic chiral adsorbent phase;
xii) extraction with chiral solvents—a technique whereby the stereoisomers are separated by virtue of preferential dissolution of one stereoisomer into a particular chiral solvent;
xiii) transport across chiral membranes—a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane which allows only one stereoisomer of the racemate to pass through.

In some or any embodiments, provided is a composition of compound that comprises a substantially pure designated stereoisomer (e.g., enantiomer, diastereomer) or geometric isomer of the compound. In some or any embodiments, in the methods and compounds disclosed herein, the compounds are substantially free of other stereoisomers (e.g., enantiomers, diastereomers) and/or geometric isomers thereof. In some or any embodiments, a composition includes a compound that is at least 85%, 90%, 95%, 98%, 99% or 100% by weight, of the specific compound, the remainder comprising other chemical species, geometric isomers, and/or stereoisomers (e.g., enantiomers, diastereomers) thereof.

### Isotopically Enriched Compounds

Also provided herein are isotopically enriched compounds.

The term “Isotopic composition,” as used herein, and unless otherwise specified, refers to the amount of each isotope present for a given atom, and “natural isotopic composition” refers to the naturally occurring isotopic composition or abundance for a given atom. Atoms containing their natural isotopic composition may also be referred to herein as “non-enriched” atoms. Unless otherwise designated, the atoms of the compounds recited herein are meant to represent any stable isotope of that atom. For example, unless otherwise stated, when a position is designated specifically as “H” or “hydrogen,” the position is understood to have hydrogen at its natural isotopic composition.

The term “isotopic enrichment,” as used herein, and unless otherwise specified, refers to the percentage of incorporation of an amount of a specific isotope at a given atom in a molecule in the place of that atom's natural isotopic abundance. In some or any embodiments, deuterium enrichment of 1% at a given position means that 1% of the molecules in a given sample contain deuterium at the specified position. Because the naturally occurring distribution of deuterium is about 0.0156%, deuterium enrichment at any position in a compound synthesized using non-enriched starting materials is about 0.0156%. The isotopic enrichment of the compounds provided herein can be determined using conventional analytical methods known to one of ordinary skill in the art, including mass spectrometry and nuclear magnetic resonance spectroscopy.

The term “isotopically enriched,” as used herein, and unless otherwise specified, refers to an atom having an isotopic composition other than the natural isotopic composition of that atom. “Isotopically enriched” may also refer to a compound containing at least one atom having an isotopic composition other than the natural isotopic composition of that atom.

Isotopic enrichment (in some or any embodiments, deuteration) of pharmaceuticals to improve pharmacokinetics (“PK”), pharmacodynamics (“PD”), and toxicity profiles, has been demonstrated previously with some classes of drugs. See, for example, Lijinsky et. al., Food Cosmet. Toxicol., 20: 393 (1982); Lijinsky et. al., J. Nat. Cancer Inst., 69: 1127 (1982); Mangold et. al., Mutation Res. 308: 33 (1994); Gordon et. al., Drug Metab. Dispos., 15: 589 (1987); Zello et. al., Metabolism, 43: 487 (1994); Gately et. al., J. Nucl. Med., 27: 388 (1986); Wade D, Chem. Biol. Interact. 117: 191 (1999)

Isotopic enrichment of a drug can be used, in some or any embodiments, to (1) reduce or eliminate unwanted metabolites, (2) increase the half-life of the parent drug, (3) decrease the number of doses needed to achieve a desired effect, (4) decrease the amount of a dose necessary to achieve a desired effect, (5) increase the formation of active metabolites, if any are formed, and/or (6) decrees the production of deleterious metabolites in specific tissues and/or create a more effective drug and/or a safer drug for combination therapy, whether the combination therapy is intentional or not.

Replacement of an atom for one of its isotopes often will result in a change in the reaction rate of a chemical reaction. This phenomenon is known as the Kinetic Isotope Effect (“KIE”). For example, if a C—H bond is broken during a rate-determining step in a chemical reaction (i.e. the step with the highest transition state energy), substitution of a deuterium for that hydrogen will cause a decrease in the reaction rate and the process will slow down. This phenomenon is known as the Deuterium Kinetic Isotope Effect (“DKIE”). See, e.g., Foster et al., Adv. Drug Res., vol. 14, pp. 1-36 (1985); Kushner et al., Can. J. Physiol. Pharmacol., vol. 77, pp. 79-88 (1999).

The magnitude of the DKIE can be expressed as the ratio between the rates of a given reaction in which a C—H bond is broken, and the same reaction where deuterium is substituted for hydrogen. The DKIE can range from about 1 (no isotope effect) to very large numbers, such as 50 or more, meaning that the reaction can be fifty, or more, times slower when deuterium is substituted for hydrogen. High DKIE values may be due in part to a phenomenon known as tunneling, which is a consequence of the uncertainty principle. Tunneling is ascribed to the small mass of a hydrogen atom, and occurs because transition states involving a proton can sometimes form in the absence of the required activation energy. Because deuterium has more mass than hydrogen, it statistically has a much lower probability of undergoing this phenomenon

Tritium (“T”) is a radioactive isotope of hydrogen, used in research, fusion reactors, neutron generators and radiopharmaceuticals. Tritium is a hydrogen atom that has 2 neutrons in the nucleus and has an atomic weight close to 3. It occurs naturally in the environment in very low concentrations, most commonly found as T₂O. Tritium decays slowly (half-life=12.3 years) and emits a low energy beta particle that cannot penetrate the outer layer of human skin. Internal exposure is the main hazard associated with this isotope, yet it must be ingested in large amounts to pose a significant health risk. As compared with deuterium, a lesser amount of tritium must be consumed before it reaches a hazardous level. Substitution of tritium (“T”) for hydrogen results in yet a stronger bond than deuterium and gives numerically larger isotope effects. Similarly, substitution of isotopes for other elements, including, but not limited to, ¹³C or ¹⁴C for carbon, ³³S, ³⁴S, or ³⁶S for sulfur, ¹⁵N for nitrogen, and ¹⁷O or ¹⁸O for oxygen, may lead to a similar kinetic isotope effect.

For example, the DKIE was used to decrease the hepatotoxicity of halothane by presumably limiting the production of reactive species such as trifluoroacetyl chloride. However, this method may not be applicable to all drug classes. For example, deuterium incorporation can lead to metabolic switching. The concept of metabolic switching asserts that xenogens, when sequestered by Phase I enzymes, may bind transiently and re-bind in a variety of conformations prior to the chemical reaction (e.g., oxidation). This hypothesis is supported by the relatively vast size of binding pockets in many Phase I enzymes and the promiscuous nature of many metabolic reactions. Metabolic switching can potentially lead to different proportions of known metabolites as well as altogether new metabolites. This new metabolic profile may impart more or less toxicity.

In some embodiments, the compounds described herein may be used as radiopharmaceuticals such as, for example, imaging agents. In one instance, radiopharmaceuticals are positron emission tomography (PET) imaging agents. In such embodiments, substitution of radionuclides (e.g., positron emitting isotopes) for atoms in the compounds allows for the syntheses of radiopharmaceuticals that can function as imaging agents. In some embodiments, radionuclides which can be substituted in the compounds described herein include, and are not limited to, ¹⁸F, ¹¹C, ¹³N, ¹⁵O, ⁷⁶Br, and ¹²⁴I. In some embodiments, the compound is isotopically enriched at one or more atoms, one atom, two atoms, or three atoms. In some embodiments, the compound is administered as an isotopic composition.

The animal body expresses a variety of enzymes for the purpose of eliminating foreign substances, such as therapeutic agents, from its circulation system. In some or any embodiments, such enzymes include the cytochrome P450 enzymes (“CYPs”), esterases, proteases, reductases, dehydrogenases, and monoamine oxidases, to react with and convert these foreign substances to more polar intermediates or metabolites for renal excretion. Some of the most common metabolic reactions of pharmaceutical compounds involve the oxidation of a carbon-hydrogen (C—H) bond to either a carbon-oxygen (C—O) or carbon-carbon (C—C) pi-bond. The resultant metabolites may be stable or unstable under physiological conditions, and can have substantially different pharmacokinetic, pharmacodynamic, and acute and long-term toxicity profiles relative to the parent compounds. For many drugs, such oxidations are rapid. These drugs therefore often require the administration of multiple or high daily doses.

Therefore, isotopic enrichment at certain positions of a compound provided herein will produce a detectable KIE that will affect the pharmacokinetic, pharmacologic, and/or toxicological profiles of a compound provided herein in comparison with a similar compound having a natural isotopic composition.

### General Schemes

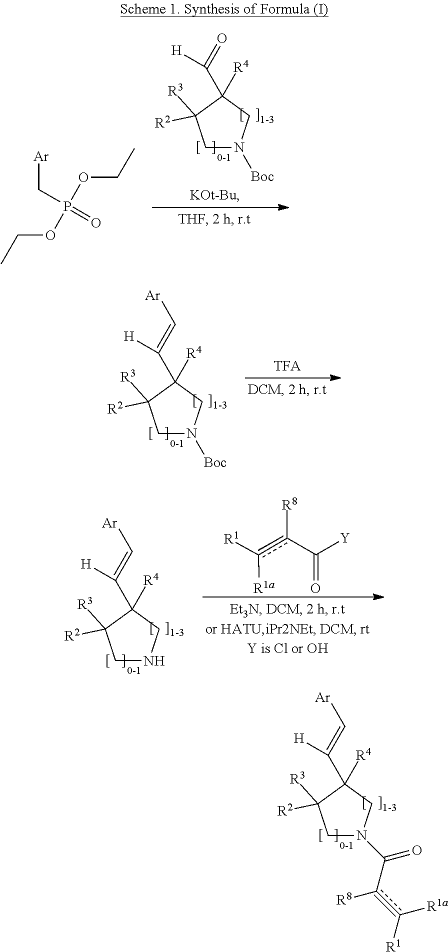

Scheme 1 illustrates a synthesis of compounds of Formula (I) according to some aspects and embodiments of the present disclosure. While only the E (trans) isomer is shown in Scheme 1, for simplicity, cis and/or trans isomers may result and can be separated by common chromatographic methods. In certain aspects, mixtures of stereoisomers (e.g., enantiomers, diastereomers) and/or geometric isomers (e.g., regioisomers, cis and trans isomers, Z and E isomers) will result, and individual stereoisomers (e.g., enantiomers, diastereomers) and/or geometric isomers (e.g., regioisomers, cis and trans isomers, Z and E isomers) can be separated using (chiral) chromatography to obtain single stereoisomers (e.g., enantiomers, diastereomers) and/or geometric isomers (e.g., regioisomers, cis and trans isomers, Z and E isomers) thereof. Compounds of Formula (I) may be prepared by other routes, and several of these are described in the experimental section.

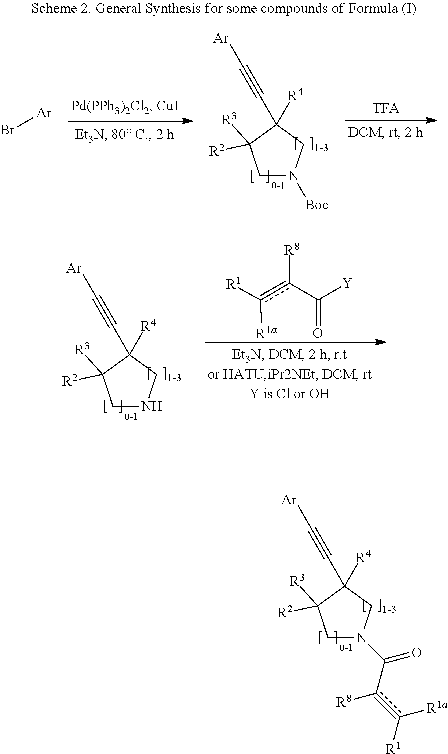

Scheme 2 illustrates a synthesis of compounds of Formula (I) according to some aspects and embodiments of the present disclosure. In certain aspects, mixtures of stereoisomers (e.g., enantiomers, diastereomers) and/or geometric isomers (e.g., regioisomers, cis and trans isomers, Z and E isomers) will result, and individual stereoisomers (e.g., enantiomers, diastereomers) and/or geometric isomers (e.g., regioisomers, cis and trans isomers, Z and E isomers) can be separated using (chiral) chromatography to obtain single stereoisomers (e.g., enantiomers, diastereomers) and/or geometric isomers (e.g., regioisomers, cis and trans isomers, Z and E isomers) thereof.

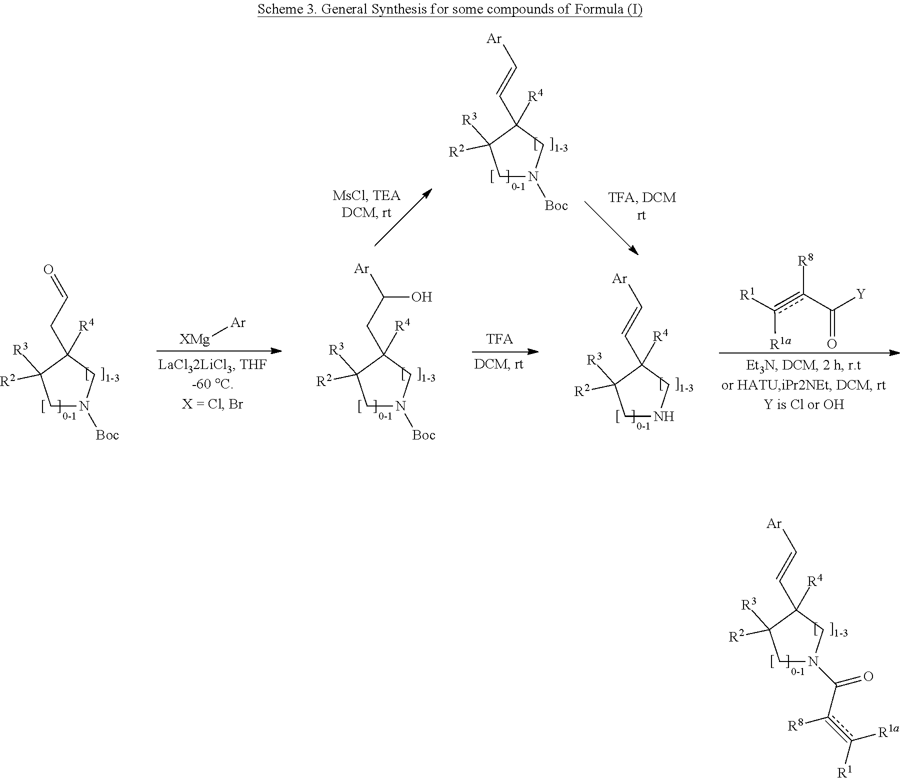

Scheme 3 illustrates a synthesis of compounds of Formula (I) according to some aspects and embodiments of the present disclosure. The aldehyde functionality in

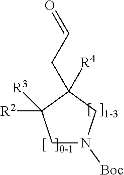

alternatively can be a cyclic ketone or lactone. While only the E isomer is shown in Scheme 3, for simplicity, one specific Z or E olefin isomer can be obtained for the double bond to which Ar is attached. In certain aspects, mixtures of stereoisomers (e.g., enantiomers, diastereomers) and/or geometric isomers (e.g., regioisomers, cis and trans isomers, Z and E isomers) will result, and individual stereoisomers (e.g., enantiomers, diastereomers) and/or geometric isomers (e.g., regioisomers, cis and trans isomers, Z and E isomers) can be separated using (chiral) chromatography to obtain single stereoisomers (e.g., enantiomers, diastereomers) and/or geometric isomers (e.g., regioisomers, cis and trans isomers, Z and E isomers) thereof.

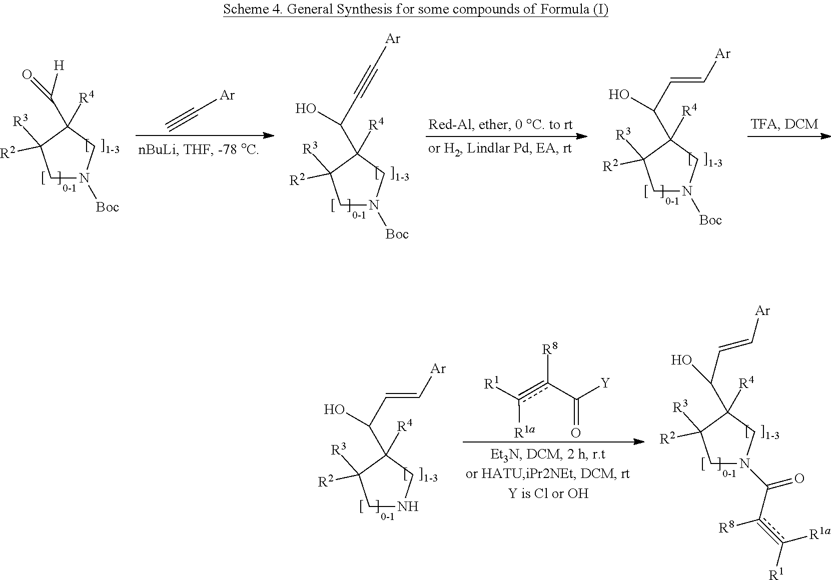

Scheme 4 illustrates a synthesis of compounds of Formula (I) according to some aspects and embodiments of the present disclosure. Under different conditions for the reduction of the alkyne functionality in the second step, one specific Z or E olefin isomer can be obtained (only E isomer is shown in Scheme 4 for simplicity). In certain aspects, mixtures of stereoisomers (e.g., enantiomers, diastereomers) and/or geometric isomers (e.g., regioisomers, cis and trans isomers, Z and E isomers) will result, and individual stereoisomers (e.g., enantiomers, diastereomers) and/or geometric isomers (e.g., regioisomers, cis and trans isomers. Z and E isomers) can be separated using (chiral) chromatography to obtain single stereoisomers (e.g., enantiomers, diastereomers) and/or geometric isomers (e.g., regioisomers, cis and trans isomers, Z and E isomers) thereof.

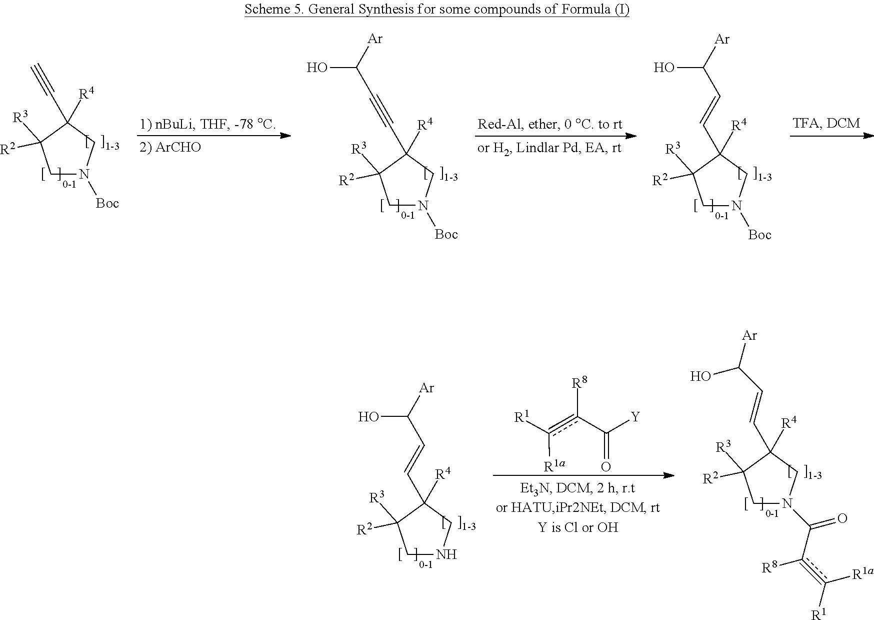

Scheme 5 illustrates a synthesis of compounds of Formula (I) according to some aspects and embodiments of the present disclosure. Under different conditions for the reduction of the alkyne functionality in the second step, one specific Z or E olefin isomer can be obtained (only E isomer is shown in Scheme 5 for simplicity). In certain aspects, mixtures of stereoisomers (e.g., enantiomers, diastereomers) and/or geometric isomers (e.g., regioisomers, cis and trans isomers, Z and E isomers) will result, and individual stereoisomers (e.g., enantiomers, diastereomers) and/or geometric isomers (e.g., regioisomers, cis and trans isomers. Z and E isomers) can be separated using (chiral) chromatography to obtain single stereoisomers (e.g., enantiomers, diastereomers) and/or geometric isomers (e.g., regioisomers, cis and trans isomers, Z and E isomers) thereof.

### Pharmaceutical Formulations and Methods of Treatment and Administration

The term “treatment” is used interchangeably herein with the term “therapeutic method” and refers to both 1) therapeutic treatments or measures that cure, slow down, lessen symptoms of, and/or halt progression of a diagnosed pathologic conditions, disease or disorder, and 2) and prophylactic/preventative measures. Those in need of treatment may include individuals already having a particular medical disease or disorder as well as those who may ultimately acquire the disorder (i.e., those needing preventive measures).

“Treating” or “treatment” of any condition or disorder refers, in some or any embodiments, to ameliorating a condition or disorder that exists in a subject, including prophylactically. In another embodiment, “treating” or “treatment” includes ameliorating at least one physical parameter, which may be indiscernible by the subject. In yet another embodiment, “treating” or “treatment” includes modulating the condition or disorder, either physically (e.g., stabilization of a discernible symptom) or physiologically (e.g., stabilization of a physical parameter) or both. In yet another embodiment, “treating” or “treatment” includes delaying the onset of the condition or disorder. In yet another embodiment, “treating” or “treatment” includes the reduction or elimination of either the condition (e.g. cancer) or one or more parameters (e.g. tumor size or tumor growth rate) of the condition (e.g. cancer), or to retard the progression of the condition (e.g. cancer) or of one or more parameters (e.g. tumor size or tumor growth rate) of the condition (e.g. cancer), or to reduce the severity of the condition (e.g. cancer) or of one or more parameters (e.g. tumor size or tumor growth rate) of the condition (e.g. cancer). In yet another embodiment. “treating” or “treatment” includes administering a compound described herein prophylactically. In some embodiments, “Treating” or “treatment” of a disease includes: (1) inhibiting the disease, i.e., arresting (i.e., stabilizing) or reducing the development of the disease or its clinical symptoms; or (2) relieving the disease, i.e., causing regression of the disease or its clinical symptoms.

The term “subject” as used herein refers to any individual or patient to which the subject methods are performed. Generally, the subject is human, although as will be appreciated by those in the art, the subject may be an animal.

The terms “therapeutically effective amount”, “effective dose”, “therapeutically effective dose”, “effective amount,” and the like refer to the amount of a subject compound or composition that will elicit the biological or medical response in a tissue, system, animal, individual, or human that is being sought by administering said compound. The biological or medical response includes one or more of the following: (1) Preventing the disease; for example, preventing a disease, condition or disorder in an individual that may be predisposed to the disease, condition or disorder but does not yet experience or display the pathology or symptomatology of the disease, (2) Inhibiting the disease; for example, inhibiting a disease, condition or disorder in an individual that is experiencing or displaying the pathology or symptomatology of the disease, condition or disorder (i.e., arresting further development of the pathology and/or symptomatology), and (3) Ameliorating the disease; for example, ameliorating a disease, condition or disorder in an individual that is experiencing or displaying the pathology or symptomatology of the disease, condition or disorder (i.e., reversing the pathology and/or symptomatology). Generally, the response is either amelioration of symptoms in a patient or a desired biological outcome.

Also disclosed herein are pharmaceutical compositions comprising compounds disclosed herein (including according to formula (I), (II), any embodiments herein, and any of Compounds 1-106). The term “pharmaceutically acceptable carrier” refers to a non-toxic carrier that may be administered to a patient, together with a compound of this disclosure, and which does not destroy the pharmacological activity thereof. Pharmaceutically acceptable carriers that may be used in these compositions include, but are not limited to, ion exchangers, alumina, aluminum stearate, lecithin, serum proteins such as human serum albumin, buffer substances such as phosphates, glycine, sorbic acid, potassium sorbate, partial glyceride mixtures of saturated vegetable fatty acids, water, salts or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Pharmaceutically acceptable carriers that may be used in the pharmaceutical compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, wool fat and self-emulsifying drug delivery systems (SEDDS) such as α-tocopherol, polyethyleneglycol 1000 succinate, or other similar polymeric delivery matrices.

In pharmaceutical composition comprising only the compounds described herein (including according to formula (I), (II), any embodiments herein, and any of Compounds 1-106) as the active component, methods for administering these compositions may additionally comprise the step of administering to the subject an additional agent or therapy. Such therapies include, but are not limited to, an anemia therapy, a diabetes therapy, a hypertension therapy, a cholesterol therapy, neuropharmacologic drugs, drugs modulating cardiovascular function, drugs modulating inflammation, immune function, production of blood cells; hormones and antagonists, drugs affecting gastrointestinal function, chemotherapeutics of microbial diseases, and/or chemotherapeutics of neoplastic disease. In another embodiment, the therapy is an anemia therapy, a diabetes therapy, a hypertension therapy, a cholesterol therapy, neuropharmacologic drugs, drugs modulating cardiovascular function, drugs modulating inflammation, immune function, production of blood cells; hormones and antagonists, drugs affecting gastrointestinal function, and/or chemotherapeutics of neoplastic disease. Other pharmacological therapies can include any other drug or biologic found in any drug class. For example, other drug classes can comprise allergy/cold/ENT therapies, analgesics, anesthetics, anti-inflammatories, antimicrobials, antivirals, asthma/pulmonary therapies, cardiovascular therapies, dermatology therapies, endocrine/metabolic therapies, gastrointestinal therapies, cancer therapies, immunology therapies, neurologic therapies, ophthalmic therapies, psychiatric therapies or rheumatologic therapies. In another embodiment, the therapy is selected from anti-inflammatories, endocrine/metabolic therapies, gastrointestinal therapies, cancer therapies, immunology therapies, neurologic therapies, and rheumatologic therapies. Other examples of agents or therapies that can be administered with the compounds described herein include a matrix metalloprotease inhibitor, a lipoxygenase inhibitor, a cytokine antagonist, an immunosuppressant, a cytokine, a growth factor, an immunomodulator, a prostaglandin or an anti-vascular hyperproliferation compound.

In another embodiment, a compound disclosed herein (including according to formula (I), (II), any embodiments herein, and any of Compounds 1-106) is used with another anti-cancer agent, such as, one or more selected from EGFR inhibitors, MEK inhibitors, KRAS G12C inhibitors, KRAS G12D inhibitors, immune checkpoint inhibitors, and chemotherapeutic agents for neoplastic disease.

The compounds of this disclosure (including according to formula (I), (II), any embodiments herein, and any of Compounds 1-106) may be employed in a conventional manner for controlling the disease described herein, including, but not limited to, cancer. Such methods of treatment, their dosage levels and requirements may be selected by those of ordinary skill in the art from available methods and techniques. For example, the compounds of this disclosure may be combined with a pharmaceutically acceptable adjuvant for administration to a patient suffering from cancer in a pharmaceutically acceptable manner and in an amount effective to treat cancer.

Alternatively, the compounds of this disclosure (including according to formula (I), (II), any embodiments herein, and any of Compounds 1-106) may be used in compositions and methods for treating or protecting individuals against the diseases described herein, including but not limited to cancer, over extended periods of time. The compounds may be employed in such compositions either alone or together with other compounds of this disclosure in a manner consistent with the conventional utilization of such compounds in pharmaceutical compositions. For example, a compound of this disclosure may be combined with pharmaceutically acceptable adjuvants conventionally employed in vaccines and administered in prophylactically effective amounts to protect individuals over an extended period of time against the diseases described herein, including, but not limited to, cancer.

As used herein, the terms “combination,” “combined,” and related terms refer to the simultaneous or sequential administration of therapeutic agents in accordance with this disclosure. For example, a described compound (including according to formula (I), (II), any embodiments herein, and any of Compounds 1-106) may be administered with another therapeutic agent simultaneously or sequentially in separate unit dosage forms or together in a single unit dosage form. Accordingly, the present disclosure provides a single unit dosage form comprising a described compound, an additional therapeutic agent, and a pharmaceutically acceptable carrier, adjuvant, or vehicle. Two or more agents are typically considered to be administered “in combination” when a patient or individual is simultaneously exposed to both agents. In many embodiments, two or more agents are considered to be administered “in combination” when a patient or individual simultaneously shows therapeutically relevant levels of the agents in a particular target tissue or sample (e.g., in brain, in serum, etc.).

When the compounds of this disclosure (including according to formula (I), (II), any embodiments herein, and any of Compounds 1-106) are administered in combination therapies with other agents, they may be administered sequentially or concurrently to the patient. Alternatively, pharmaceutical or prophylactic compositions according to this disclosure comprise a combination of a compound described herein (including according to formula (I), (II), any embodiments herein, and any of Compounds 1-106), and another therapeutic or prophylactic agent. Additional therapeutic agents that are normally administered to treat a particular disease or condition may be referred to as “agents appropriate for the disease, or condition, being treated.”

The compounds utilized in the compositions and methods of this disclosure may also be modified by appending appropriate functionalities to enhance selective biological properties. Such modifications are known in the art and include those, which increase biological penetration into a given biological system (e.g., blood, lymphatic system, or central nervous system), increase oral availability, increase solubility to allow administration by injection, alter metabolism and/or alter rate of excretion.

According to an embodiment, the compositions of this disclosure are formulated for pharmaceutical administration to a subject or patient. e.g., a mammal, preferably a human being. Such pharmaceutical compositions are used to ameliorate, treat or prevent any of the diseases described herein including but not limited to cancer in a subject.

Agents of the disclosure are often administered as pharmaceutical compositions comprising an active therapeutic agent. i.e., and a variety of other pharmaceutically acceptable components. See Remington's Pharmaceutical Science (15th ed., Mack Publishing Company, Easton, Pa., 1980). The preferred form depends on the intended mode of administration and therapeutic application. The compositions can also include, depending on the formulation desired, pharmaceutically acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

In some embodiments, the present disclosure provides pharmaceutically acceptable compositions comprising a therapeutically effective amount of one or more of a described compound (including according to formula (I), (II), any embodiments herein, and any of Compounds 1-106), formulated together with one or more pharmaceutically acceptable carriers (additives) and/or diluents for use in treating the diseases described herein, including, but not limited to cancer. While it is possible for a described compound to be administered alone, it is preferable to administer a described compound (including according to formula (I), (II), any embodiments herein, and any of Compounds 1-106) as a pharmaceutical formulation (composition) as described herein. Described compounds (including according to formula (I), (II), any embodiments herein, and any of Compounds 1-106) may be formulated for administration in any convenient way for use in human or veterinary medicine, by analogy with other pharmaceuticals.

As described in detail, pharmaceutical compositions of the present disclosure may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream or foam; sublingually; ocularly; transdermally; or nasally, pulmonary and to other mucosal surfaces.

Wetting agents, emulsifiers and lubricants, such as sodium laurel sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Formulations for use in accordance with the present disclosure include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient, which can be combined with a carrier material, to produce a single dosage form will vary depending upon the host being treated, and the particular mode of administration. The amount of active ingredient that can be combined with a carrier material to produce a single dosage form will generally be that amount of the compound, which produces a therapeutic effect. Generally, this amount will range from about 1% to about 99% of active ingredient. In some embodiments, this amount will range from about 5% to about 70%, from about 10% to about 50%, or from about 20% to about 40%.

In certain embodiments, a formulation as described herein comprises an excipient selected from the group consisting of cyclodextrins, liposomes, micelle forming agents, e.g., bile acids, and polymeric carriers, e.g., polyesters and polyanhydrides; and a compound of the present disclosure (including according to formula (I), (II), any embodiments herein, and any of Compounds 1-106). In certain embodiments, an aforementioned formulation renders orally bioavailable a described compound of the present disclosure (including according to formula (I), (II), any embodiments herein, and any of Compounds 1-106).

Methods of preparing formulations or compositions comprising described compounds (including according to formula (I), (II), any embodiments herein, and any of Compounds 1-106) include a step of bringing into association a compound of the present disclosure (including according to formula (I), (II), any embodiments herein, and any of Compounds 1-106) with the carrier and, optionally, one or more accessory ingredients. In general, formulations may be prepared by uniformly and intimately bringing into association a compound of the present disclosure (including according to formula (I), (II), any embodiments herein, and any of Compounds 1-106) with liquid carriers, or finely divided solid carriers, or both, and then, if necessary, shaping the product.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as those described in Pharmacopeia Helvetica, or a similar alcohol. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

In some cases, in order to prolong the effect of a drug, it may be desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the described compounds (including according to formula (I), (II), any embodiments herein, and any of Compounds 1-106) in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissue.

The pharmaceutical compositions of this disclosure may be orally administered in any orally acceptable dosage form including, but not limited to, capsules, tablets, and aqueous suspensions and solutions. In the case of tablets for oral use, carriers, which are commonly used include lactose and corn starch. Lubricating agents, such as magnesium stearate, are also typically added. For oral administration in a capsule form, useful diluents include lactose and dried cornstarch. When aqueous suspensions and solutions and propylene glycol are administered orally, the active ingredient is combined with emulsifying and suspending agents. If desired, certain sweetening and/or flavoring and/or coloring agents may be added.

Formulations described herein suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present disclosure (including according to formula (I), (II), any embodiments herein, and any of Compounds 1-106) as an active ingredient. Compounds described herein (including according to formula (I), (II), any embodiments herein, and any of Compounds 1-106) may also be administered as a bolus, electuary or paste.

In solid dosage forms for oral administration (capsules, tablets, pills, dragees, powders, granules and the like), an active ingredient is mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol, glycerol monostearate, and non-ionic surfactants; absorbents, such as kaolin and bentonite clay; lubricants, such as talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

Tablets may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made in a suitable machine in which a mixture of the powdered compound is moistened with an inert liquid diluent. If a solid carrier is used, the preparation can be in tablet form, placed in a hard gelatin capsule in powder or pellet form, or in the form of a troche or lozenge. The amount of solid carrier will vary, e.g., from about 25 to 800 mg, preferably about 25 mg to 400 mg. When a liquid carrier is used, the preparation can be, e.g., in the form of a syrup, emulsion, soft gelatin capsule, sterile injectable liquid such as an ampule or nonaqueous liquid suspension. Where the composition is in the form of a capsule, any routine encapsulation is suitable, for example, using the aforementioned carriers in a hard gelatin capsule shell.

Tablets and other solid dosage forms, such as dragees, capsules, pills and granules, may optionally be scored or prepared with coatings and shells, such as enteric coatings and other coatings well known in the pharmaceutical-formulating art. They may alternatively or additionally be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of compounds of the disclosure (including according to formula (I), (II), any embodiments herein, and any of Compounds 1-106) include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, oral compositions can also include adjuvants such as wetting agents, emulsifying and suspending agents, sweetening, flavoring, coloring, perfuming and preservative agents.

Suspensions, in addition to active compounds (including according to formula (I), (II), any embodiments herein, and any of Compounds 1-106), may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

The pharmaceutical compositions of this disclosure may also be administered in the form of suppositories for rectal administration. These compositions can be prepared by mixing a compound of this disclosure (including according to formula (I), (II), any embodiments herein, and any of Compounds 1-106) with a suitable non-irritating excipient, which is solid at room temperature but liquid at the rectal temperature and therefore will melt in the rectum to release the active components. Such materials include, but are not limited to, cocoa butter, beeswax and polyethylene glycols. Topical administration of the pharmaceutical compositions of this disclosure is especially useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this disclosure (including according to formula (I), (II), any embodiments herein, and any of Compounds 1-106) include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate-60, cetyl esters wax, cetearyl alcohol, 2-octyldodecanol, benzyl alcohol and water. The pharmaceutical compositions of this disclosure may also be topically applied to the lower intestinal tract by rectal suppository formulation or in a suitable enema formulation. Topically-administered transdermal patches are also included in this disclosure.

The pharmaceutical compositions of this disclosure may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

For ophthalmic use, the pharmaceutical compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutical compositions may be formulated in an ointment such as petrolatum.

Transdermal patches have the added advantage of providing controlled delivery of a compound of the present disclosure (including according to formula (I), (II), any embodiments herein, and any of Compounds 1-106) to the body. Dissolving or dispersing the compound in the proper medium can make such dosage forms. Absorption enhancers can also be used to increase the flux of the compound across the skin. Either providing a rate controlling membrane or dispersing the compound in a polymer matrix or gel can control the rate of such flux.

Examples of suitable aqueous and nonaqueous carriers, which may be employed in the pharmaceutical compositions of the disclosure, include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Such compositions may also contain adjuvants such as preservatives, wetting agents, emulsifying agents and dispersing agents. Inclusion of one or more antibacterial and/or antifungal agents, for example, paraben, chlorobutanol, phenol sorbic acid, and the like, may be desirable in certain embodiments. It may alternatively or additionally be desirable to include isotonic agents, such as sugars, sodium chloride, and the like into the compositions. In addition, prolonged absorption of the injectable pharmaceutical form may be brought about by the inclusion of agents, which delay absorption such as aluminum monostearate and gelatin.

In certain embodiments, a described compound (including according to formula (I), (II), any embodiments herein, and any of Compounds 1-106) or pharmaceutical preparation is administered orally. In other embodiments, a described compound (including according to formula (I), (II), any embodiments herein, and any of Compounds 1-106) or pharmaceutical preparation is administered intravenously. Alternative routes of administration include sublingual, intramuscular, and transdermal administrations.

When compounds described herein (including according to formula (I), (II), any embodiments herein, and any of Compounds 1-106) are administered as pharmaceuticals, to humans and animals, they can be given per se or as a pharmaceutical composition containing, for example, 0.1% to 99.5% of active ingredient in combination with a pharmaceutically acceptable carrier. In some embodiments, 0.5% to 90% of active ingredient can be used.

Preparations described herein may be given orally, nasally, as by, for example, a spray, parenterally, intravaginally, intracisternally, rectally, or topically, as by powders, ointments or drops, including buccally and sublingually. They are of course given in forms suitable for the relevant administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

Regardless of the route of administration selected, compounds described herein (including according to formula (I), (II), any embodiments herein, and any of Compounds 1-106) which may be used in a suitable hydrated form, and/or the pharmaceutical compositions of the present disclosure, are formulated into pharmaceutically acceptable dosage forms by conventional methods known to those of skill in the art.

Actual dosage levels of the active ingredients in the pharmaceutical compositions of the disclosure may be varied so as to obtain an amount of the active ingredient that is effective to achieve the desired therapeutic response for a particular patient, composition, and mode of administration, without being toxic to the patient.

The terms “administration of” and or “administering” should be understood to mean providing a pharmaceutical composition in a therapeutically effective amount to the subject in need of treatment. Administration routes can be enteral, topical or parenteral. As such, administration routes include but are not limited to intracutaneous, subcutaneous, intravenous, intraperitoneal, intraarterial, intrathecal, intracapsular, intraorbital, intracardiac, intradermal, transdermal, transtracheal, subcuticular, intraarticular, subcapsular, subarachnoid, intraspinal and intrasternal, oral, sublingual buccal, rectal, vaginal, nasal ocular administrations, as well infusion, inhalation, and nebulization.

The term “cancer” refers to a group of diseases characterized by abnormal and uncontrolled cell proliferation starting at one site (primary site) with the potential to invade and to spread to others sites (secondary sites, metastases) which differentiate cancer (malignant tumor) from benign tumor. Virtually all the organs can be affected, leading to more than 100 types of cancer that can affect humans. Cancers can result from many causes including genetic predisposition, viral infection, exposure to ionizing radiation, exposure environmental pollutant, tobacco and or alcohol use, obesity, poor diet, lack of physical activity or any combination thereof.

Exemplary cancers described by the national cancer institute include: Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood; Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma, Childhood Brain Stem; Glioma, Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's; Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplasia Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood’, Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland Cancer, Childhood; Sarcoma, Ewing's Family of Tumors; Sarcoma, Kaposi's; Sarcoma (Osteosarcoma Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor.

In certain aspects, cancer include Lung cancer, Breast cancer, Colorectal cancer, Prostate cancer, Stomach cancer, Liver cancer, cervical cancer, Esophageal cancer, Bladder cancer, Non-Hodgkin lymphoma, Leukemia, Pancreatic cancer, Kidney cancer, endometrial cancer, Head and neck cancer, Lip cancer, oral cancer, Thyroid cancer, Brain cancer, Ovary cancer, Melanoma, Gallbladder cancer, Laryngeal cancer, Multiple myeloma, Nasopharyngeal cancer, Hodgkin lymphoma, Testis cancer and Kaposi sarcoma.

The compounds of the disclosure (including according to formula (I), (II), any embodiments herein, and any of Compounds 1-106) can be administered in combination with one or more additional therapeutic agents. The phrases “combination therapy”, “combined with” and the like refer to the use of more than one medication or treatment simultaneously to increase the response. The TEAD1, 2, 3, and/or 4 inhibitor of the present disclosure might for example be used in combination with other drugs or treatment in use to treat cancer. In various aspects, the compound (including according to formula (I), (II), any embodiments herein, and any of Compounds 1-106) is administered prior to, simultaneously with or following the administration of the chemotherapeutic agent.

Anti-cancer therapies which could be used in combination with the compounds disclosed herein refer to any therapy or treatment that can be used for the treatment of a cancer. Anti-cancer therapies include, but are not limited to, small molecule or large molecule therapies, surgery, radiotherapy, chemotherapy, immune therapy and targeted therapies. In some or any embodiments, the additional anti-cancer therapy is a KRAS G12C and/or G12D inhibitor; in some embodiments, the KRAS G12C and/or G12D inhibitor is sotorasib or adagrasib.

In some or any embodiments, combination therapy can include, but is not limited to, compounds disclosed herein (including any of Compounds 1-106) administered in combination with one or more CDK4/6 inhibitors; one or more EGFR inhibitors; one or more RAF inhibitors; one or more MEK inhibitors; one or more Wnt signaling inhibitors, such as anti-β-catenin inhibitors, GSK3 inhibitors, JNK inhibitors, and CKT inhibitors; one or more TGF-β signaling inhibitors, such as atezolizumab, durvalumab, and avelumab; one or more PD-1/PD-L1 inhibitors; and/or radiotherapy.

Examples of chemotherapeutic agents or anti-cancer agents include, but are not limited to, Actinomycin, Azacitidine, Azathioprine, Bleomycin, Bortezomib, Carboplatin, Capecitabine, Cisplatin, Chlorambucil, Cyclophosphamide, Cytarabine, Daunorubicin, Docetaxel, Doxifluridine, Doxorubicin, Epirubicin, Epothilone, Etoposide, Fiuorouracil, Gemcitabine, Hydroxyurea, Idarubicin, Imatinib, irinotecan, Mechlorethamine, Mercaptopurine, Methotrexate, Mitoxantrone, Oxaliplatin, Paclitaxel, Pemetrexed, Teniposide, Tioguanine, Topotecan, Valrubicin, Vinblastine, Vincristine, Vindesine, Vinorelbine, panitumamab, Erbitux (cetuximab), matuzumab, IMC-IIF 8, TheraCIM hR3, denosumab, Avastin (bevacizumab), Humira (adalimumab), Herceptin (trastuzumab), Remicade (infliximab), rituximab, Synagis (palivizumab), Mylotarg (gemtuzumab oxogamicin), Raptiva (efalizumab), Tysabri (natalizumab), Zenapax (dacliximab), NeutroSpec (Technetium (99mTc) fanolesomab), tocilizumab, ProstaScint (Indium-Ill labeled Capromab Pendetide), Bexxar (tositumomab), Zevalin (ibritumomab tiuxetan (IDEC-Y2B8) conjugated to yttrium 90), Xolair (omalizumab), MabThera (Rituximab), ReoPro (abciximab), MabCampath (alemtuzumab), Simulect (basiliximab), LeukoScan (sulesomab), CEA-Scan (arcitumomab), Verluma (nofetumomab), Panorex (Edrecolomab), alemtuzumab, CDP 870, natalizumab Gilotrif (afatinib), Lynparza (olaparib), Perjeta (pertuzumab), Otdivo (nivolumab), Bosulif (bosutinib), Cabometyx (cabozantinib), Ogivri (trastuzumab-dkst), Sutent (sunitinib malate), Adcetris (brentuximab vedotin), Alecensa (alectinib), Calquence (acalabrutinib), Yescarta (ciloleucel), Verzenio (abemaciclib), Keytruda (pembrolizumab), Aligopa (copanlisib), Nerlynx (neratinib), Imfinzi (durvalumab), Darzalex (daratumumab), Tecentriq (atezolizumab), and Tarceva (erlotinib). Examples of immunotherapeutic agent include, but are not limited to, interleukins (Il-2, Il-7, Il-12), cytokines (Interferons, G-CSF, imiquimod), chemokines (CCL3, CC126, CXCL7), immunomodulatory imide drugs (thalidomide and its analogues).

In another embodiment, the present disclosure is directed to the use of compounds of Formula (I) or a stereoisomer, and/or a pharmaceutically acceptable salt thereof, and/or a solvate thereof in the treatment of any cancer indication where YAP is localized in the nucleus of the tumor cells, including, but is not limited to, lung, thyroid, ovarian, colorectal, prostate, pancreas, esophagus, liver, breast, and skin cancer.

In still another embodiment, the disclosed compounds (including according to formula (I), (II), any embodiments herein, and any of Compounds 1-106) or pharmaceutical composition thereof can disrupt YAP interactions with TEAD1, 2, 3, and/or 4. In certain embodiments, the disclosed compounds (including according to formula (I), (II), any embodiments herein, and any of Compounds 1-106) or pharmaceutical composition thereof can prevent YAP from binding to TEAD1, 2, 3, and/or 4. In some embodiments, the disclosed compounds (including according to formula (I), (II), any embodiments herein, and any of Compounds 1-106) or pharmaceutical composition thereof can compete with YAP for binding to TEAD1, 2, 3, and/or 4. In some embodiments, the disclosed compounds (including according to formula (I), (II), any embodiments herein, and any of Compounds 1-106) or pharmaceutical composition thereof can bind to TEAD1, 2, 3, and/or 4. In some embodiments, the disclosed compounds or pharmaceutical composition thereof can bind to TEAD1. In some embodiments, the disclosed compounds or pharmaceutical composition thereof can selectively bind to TEAD1 over that of the other TEAD isoforms.

By “pharmaceutically acceptable” it is meant the carrier, diluent or excipient must be compatible with the other ingredients of the formulation and not deleterious to the recipient thereof. For example, the carrier, diluent, or excipient or composition thereof may be administered to a subject along with a TEAD1, 2, 3, and/or 4 inhibitor of the disclosure (including according to formula (I), (II), any embodiments herein, and any of Compounds 1-106) without causing any undesirable biological effects or interacting in an undesirable manner with the TEAD1, 2, 3, and/or 4 inhibitor of the pharmaceutical composition in which it is contained.

In treatment, the dose of agent optionally ranges from about 0.0001 mg/kg to 100 mg/kg, about 0.01 mg/kg to 5 mg/kg, about 0.15 mg/kg to 3 mg/kg, 0.5 mg/kg to 2 mg/kg and about 1 mg/kg to 2 mg/kg of the subject's body weight. In other embodiments the dose ranges from about 100 mg/kg to 5 g/kg, about 500 mg/kg to 2 mg/kg and about 750 mg/kg to 1.5 g/kg of the subject's body weight. For example, depending on the type and severity of the disease, about 1 μg/kg to 15 mg/kg (e.g., 0.1-20 mg/kg) of agent is a candidate dosage for administration to the patient, whether, for example, by one or more separate administrations, or by continuous infusion. A typical daily dosage is in the range from about 1 μg/kg to 100 mg/kg or more, depending on the factors mentioned above. For repeated administrations over several days or longer, depending on the condition, the treatment is sustained until a desired suppression of disease symptoms occurs. However, other dosage regimens may be useful. Unit doses can be in the range, for instance of about 5 mg to 500 mg, such as 50 mg, 100 mg, 150 mg, 200 mg, 250 mg and 300 mg. The progress of therapy is monitored by conventional techniques and assays.

In some embodiments, an agent is administered to a human patient at an effective amount (or dose) of less than about 1 μg/kg, for instance, about 0.35 to 0.75 μg/kg or about 0.40 to 0.60 μg/kg. In some embodiments, the dose of an agent is about 0.35 μg/kg, or about 0.40 μg/kg, or about 0.45 μg/kg, or about 0.50 μg/kg, or about 0.55 μg/kg, or about 0.60 μg/kg, or about 0.65 μg/kg, or about 0.70 μg/kg, or about 0.75 μg/kg, or about 0.80 μg/kg, or about 0.85 μg/kg, or about 0.90 μg/kg, or about 0.95 μg/kg or about 1 μg/kg. In various embodiments, the absolute dose of an agent is about 2 μg/subject to 45 μg/subject, or about 5 to 40, or about 10 to 30, or about 15 to 25 μg/subject. In some embodiments, the absolute dose of an agent is about 20 μg, or about 30 μg, or about 40 μg.

In various embodiments, the dose of an agent may be determined by the human patient's body weight. For example, an absolute dose of an agent of about 2 μg for a pediatric human patient of about 0 to 5 kg (e.g. about 0, or about 1, or about 2, or about 3, or about 4, or about 5 kg); or about 3 μg for a pediatric human patient of about 6 to 8 kg (e.g. about 6, or about 7, or about 8 kg), or about 5 μg for a pediatric human patient of about 9 to 13 kg (e.g. 9, or about 10, or about 11, or about 12, or about 13 kg); or about 8 μg for a pediatric human patient of about 14 to about 20 kg (e.g. about 14, or about 16, or about 18, or about 20 kg), or about 12 μg for a pediatric human patient of about 21 to about 30 kg (e.g. about 21, or about 23, or about 25, or about 27, or about 30 kg), or about 13 μg for a pediatric human patient of about 31 to about 33 kg (e.g. about 31, or about 32, or about 33 kg), or about 20 μg for an adult human patient of about 34 to about 50 kg (e.g. about 34, or about 36, or about 38, or about 40, or about 42, or about 44, or about 46, or about 48, or about 50 kg), or about 30 μg for an adult human patient of about 51 to about 75 kg (e.g. about 51, or about 55, or about 60, or about 65, or about 70, or about 75 kg), or about 45 μg for an adult human patient of greater than about 114 kg (e.g. about 114, or about 120, or about 130, or about 140, or about 150 kg).

In certain embodiments, an agent in accordance with the methods provided herein is administered subcutaneously (s.c.), intravenously (i.v.), intramuscularly (i.m.), intranasally or topically. Administration of an agent described herein (including according to formula (I), (II), any embodiments herein, and any of Compounds 1-106) can, independently, be one to four times daily or one to four times per month or one to six times per year or once every two, three, four or five years. Administration can be for the duration of one day or one month, two months, three months, six months, one year, two years, three years, and may even be for the life of the human patient. The dosage may be administered as a single dose or divided into multiple doses. In some embodiments, an agent is administered about 1 to 3 times (e.g. 1, or 2 or 3 times).

### SYNTHETIC EXAMPLES

Presented below are examples discussing synthesis and characterization of TEAD1, 2, 3, and/or 4 inhibitors contemplated for the discussed applications. The following examples are provided to further illustrate the embodiments of the present invention, but are not intended to limit the scope of the invention. While they are typical of those that might be used, other procedures, methodologies, or techniques known to those skilled in the art may alternatively be used.

As used herein, the symbols and conventions used in these processes, schemes and examples, regardless of whether a particular abbreviation is specifically defined, are consistent with those used in the contemporary scientific literature, for example, the Journal of the American Chemical Society or the Journal of Biological Chemistry. Specifically, but without limitation, the following abbreviations may be used in the examples and throughout the specification: aq. (aqueous); calcd. (calculated); avg. (average); ESI (electrospray ionization); HPLC (high pressure liquid chromatography); g (grams); mg (milligrams); mL (milliliters); μL (microliters); mM (millimolar); μM (micromolar); Hz (Hertz); MHz (megahertz); mmol (millimoles); h, hr, or hrs (hours); min (minutes); MS (mass spectrometry); ppm (parts per million); r.t. or rt (room temperature); sat. (saturated); TLC (thin layer chromatography); ACN (acetonitrile); BOC (t-butyloxycarbonyl); DCM (dichloromethane); DIEA (di-isopropylethylamine); DMSO (dimethylsulfoxide); DMSO-d₆ (deuterated dimethylsulfoxide); dppf (1,1′-bis(diphenylphosphino)ferrocene); EA (ethyl acetate); Hex (hexanes), EDCI (1-ethyl-3-(3-dimethylaminopropyl)carbodiimide); EtOAc (ethyl acetate); IPA (isopropyl acetate); MeOH (methanol); MsCl (mesyl chloride); MTBE (Methyl tert-butyl ether); PE (petroleum ether); TEA (triethylamine); HATU (hexafluorophosphate azabenzotriazole tetramethyl Uronium); HOBT (hydroxybenzotriazole), Red-Al (sodium bis(2-methoxyethoxy)aluminium hydride), DMF (dimethylformamide), TBAF (tetra-n-butylammonium fluoride), LiHMDS (Lithium bis(trimethylsilyl)amide), HMPA (Hexamethylphosphoramide), AcOH (acetic acid), TMSOTf (trimethylsilyl trifluoromethanesulfonate), mCPBA (meta-Chloroperoxybenzoic acid), DIBALH (diisobutylaluminium hydride), LDA (lithium diisopropylamide), TFA (trifluoroacetic acid); and THF (tetrahydrofuran).

For all of the following examples, standard work-up and purification methods known to those skilled in the art can be utilized. Unless otherwise indicated, all temperatures are expressed in ° C. (degrees Celsius). All reactions are conducted at room temperature unless otherwise noted. Synthetic methodologies illustrated herein are intended to exemplify the applicable chemistry through the use of specific examples and are not indicative of the scope of the disclosure.

### Example 1: Synthesis of 1-{3-[(2Z)-3-[5-(trifluoromethyl)-1,2-oxazol-3-yl]prop-2-en-1-yl]azetidin-1-yl}prop-2-en-1-one

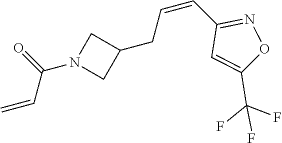 [SMILES: C=CC(=O)N1CC(C/C=C\c2cc(C(F)(F)F)on2)C1]

### Synthetic Route:

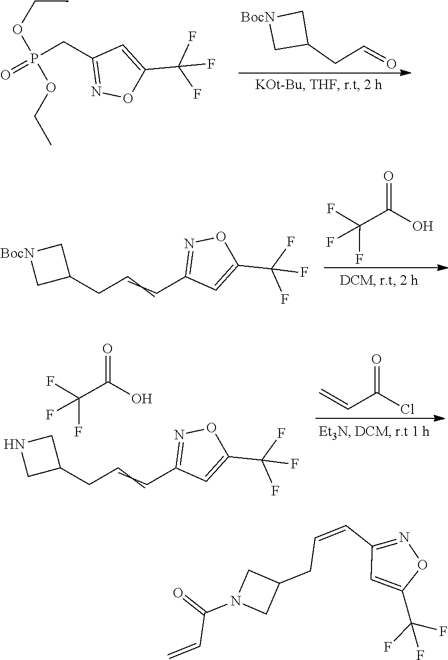

### Synthesis of tert-butyl 3-{3-[5-(trifluoromethyl)-1,2-oxazol-3-yl]prop-2-en-1-yl}azetidine-1-carboxylate

Diethyl [5-(trifluoromethyl)-1,2-oxazol-3-yl]methylphosphonate (300 mg, 1.045 mmol), tert-butyl 3-(2-oxoethyl)azetidine-1-carboxylate (250 mg, 1.255 mmol), a stir bar, and THF (10 mL) were added to a 50 mL round-bottom and stirred until homogeneous, and then treated with potassium tert-butoxide (176 mg, 1.568 mmol) at 0° C. The reaction mixture was stirred for 2 h at r.t, then diluted with water (30 mL), and extracted with EtOAc (50 mL×2). The combined extracts were dried over anhydrous sodium sulfate, filtered, and concentrated to dryness. The residue was purified on a silica gel column eluted with EA/PE (0-20%) to afford tert-butyl 3-{3-[5-(trifluoromethyl)-1,2-oxazol-3-yl]prop-2-en-1-yl}azetidine-1-carboxylate as a light yellow oil. MS (ESI) mass calcd. for C₁₅H₁₉F₃N₂O₃, 332.13 m/z, found, 277.00 [M+H−56]⁺.

### Synthesis of 3-[3-(azetidin-3-yl)prop-1-en-1-yl]-5-(trifluoromethyl)-1,2-oxazole 2,2,2-trifluoroacetate

Tert-butyl 3-{3-[5-(trifluoromethyl)-1,2-oxazol-3-yl]prop-2-en-1-yl}azetidine-1-carboxylate (270 mg, 0.812 mmol), a stir bar, and DCM (10 mL) were added to a 50 mL round-bottom flask and stirred until homogeneous, and then treated with TFA (2 mL). The reaction mixture was stirred for 2 h at r.t., then concentrated under vacuum to afford 3-[3-(azetidin-3-yl)prop-1-en-1-yl]-5-(trifluoromethyl)-1,2-oxazole 2,2,2-trifluoroacetate as a light brown oil (300 mg, crude). MS (ESI) mass calcd. for C₁₀H₁₁F₃N₂O, 232.08 m/z, found, 233.05 [M+H]⁺.

### Synthesis of 1-{3-[(2Z)-3-[5-(trifluoromethyl)-1,2-oxazol-3-yl]prop-2-en-1-yl]azetidin-1-yl}prop-2-en-1-one

3-[3-(azetidin-3-yl)prop-1-en-1-yl]-5-(trifluoromethyl)-1,2-oxazole 2,2,2-trifluoroacetate (300 mg, 0.866 mmol), a stir bar, Et₃N (438 mg, 4.328 mmol), DCM (8 mL) were added to a 50 mL round-bottom flask and stirred until homogeneous, then treated with a solution of acryloyl chloride (118 mg, 1.304 mmol) in DCM (2 mL) dropwise. The mixture was stirred at r.t. for 1 h, then quenched with water (30 mL) and extracted with DCM (50 mL×3). The combined organic extracts were washed with brine, dried over anhydrous Na₂SO₄, filtered and concentrated under vacuum. The residue was purified by HPLC with (Column: XBridge Prep OBD C18 Column, 30*150 mm, 5 μm; Mobile Phase A: Water (0.1% FA), Mobile Phase B: ACN; Flow rate: 60 mL/min; Gradient: 30% B to 60% B in 7 min, 60% B; Wave Length: 254 nm; RT1(min): 6) to afford a mixture which was further separated by Chiral HPLC with (Column: CHIRAL ART Cellulose-SB, 2*25 cm, 5 μm; Mobile Phase A: Hex (0.5% 2M NH₃-MeOH)—HPLC, Mobile Phase B: EtOH—HPLC; Flow rate: 20 mL/min; Gradient: 10% B to 10% B in 20 min; Wave Length: 220/254 nm; RT1(min): 14.806; Sample Solvent: EtOH—HPLC; Injection Volume: 0.4 mL; Number Of Runs: 9) to afford 1-{3-[(2Z)-3-[5-(trifluoromethyl)-1,2-oxazol-3-yl]prop-2-en-1-yl]azetidin-1-yl}prop-2-en-1-one as a white solid (, MS (ESI) mass calcd. for C₁₃H₁₃F₃N₂O₂, 286.09 m/z, found, 287.05 [M+H]⁺, ¹H NMR (300 MHz, DMSO-d₆) δ 7.59 (q, J=1.1 Hz, 1H), 6.46 (dt, J=11.7, 1.5 Hz, 1H), 6.29 (dd, J=17.0, 10.2 Hz, 1H), 6.21-6.12 (m, 1H), 6.08 (dd, J=17.0, 2.4 Hz, 1H), 5.65 (dd, J=10.2, 2.4 Hz, 1H), 4.35-4.26 (m, 1H), 4.06-3.97 (m, 1H), 3.94-3.85 (m, 1H), 3.66-3.56 (m, 1H), 2.84-2.71 (m, 3H) ¹⁹F NMR (282 MHz, DMSO-d₆) δ (ppm): −62.98).

### Example 2: Synthesis of 1-{3-[(2E)-3-[5-(trifluoromethyl)-1,2-oxazol-3-yl]prop-2-en-1-yl]azetidin-1-yl}prop-2-en-1-one

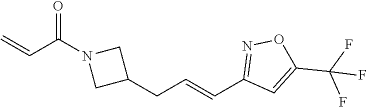 [SMILES: C=CC(=O)N1CC(C/C=C/c2cc(C(F)(F)F)on2)C1]

### Synthetic Route:

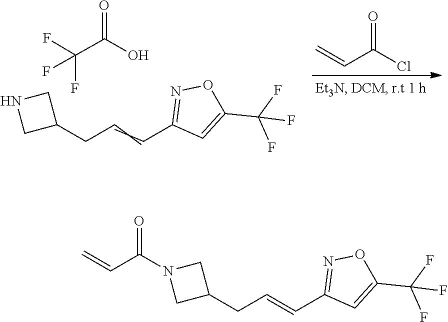

### Synthesis of 1-{3-[(2E)-3-[5-(trifluoromethyl)-1,2-oxazol-3-yl]prop-2-en-1-yl]azetidin-1-yl}prop-2-en-1-one

3-[3-(azetidin-3-yl)prop-1-en-1-yl]-5-(trifluoromethyl)-1,2-oxazole 2,2,2-trifluoroacetate (300 mg, 0.866 mmol), a stir bar, Et₃N (438 mg, 4.328 mmol), DCM (8 mL) were added to a 50 mL round-bottom flask and stirred until homogeneous, then treated with a solution of acryloyl chloride (118 mg, 1.304 mmol) in DCM (2 mL) dropwise. The mixture was stirred at r.t. for 1 h, then quenched with water (30 mL) and extracted with DCM (50 mL×3). The combined organic extracts were washed with brine, dried over anhydrous Na₂SO₄, filtered and concentrated under vacuum. The residue was purified by HPLC with (Column: XBridge Prep OBD C18 Column, 30*150 mm, 5 μm; Mobile Phase A: Water (0.1% FA), Mobile Phase B: ACN; Flow rate: 60 mL/min; Gradient: 30% B to 60% B in 7 min, 60% B; Wave Length: 254 nm; RT1(min): 6) to afford a mixture which was further separated by Chiral HPLC with (Column: CHIRAL ART Cellulose-SB, 2*25 cm, 5 μm; Mobile Phase A: Hex (0.5% 2M NH₃-MeOH)—HPLC, Mobile Phase B: EtOH—HPLC; Flow rate: 20 mL/min; Gradient: 10% B to 10% B in 20 min; Wave Length: 220/254 nm; RT2(min): 16.998; Sample Solvent: EtOH—HPLC; Injection Volume: 0.4 mL; Number Of Runs: 9) to afford 1-{3-[(2E)-3-[5-(trifluoromethyl)-1,2-oxazol-3-yl]prop-2-en-1-yl]azetidin-1-yl}prop-2-en-1-one as a white solid (MS (ESI) mass calcd. for C₁₃H₁₃F₃N₂O₂, 286.09 m/z, found, 287.10 [M+H]⁺, ¹H NMR (300 MHz, DMSO-d₆) δ 7.79 (q, J=1.1 Hz, 1H), 6.74 (dt, J=16.1, 6.5 Hz, 1H), 6.59 (dt, J=16.1, 1.3 Hz, 1H), 6.31 (dd, J=17.0, 10.2 Hz, 1H), 6.09 (dd, J=17.0, 2.4 Hz, 1H), 5.65 (dd, J=10.2, 2.4 Hz, 1H), 4.36-4.28 (m, 1H), 4.09-3.99 (m, 1H), 3.89 (dd, J=8.6, 5.5 Hz, 1H), 3.62 (dd, J=10.0, 5.6 Hz, 1H), 2.87-2.71 (m, 1H), 2.62-2.54 (m, 2H) ¹⁹F NMR (282 MHz, DMSO-d₆) δ (ppm): −63.31)

### Example 3: Synthesis of 1-{3-[(E)-2-[3-methoxy-4-(trifluoromethyl) phenyl]ethenyl]azetidin-1-yl}prop-2-en-1-one

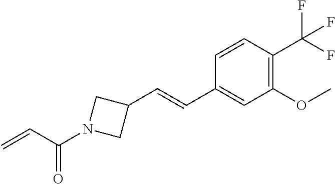 [SMILES: C=CC(=O)N1CC(/C=C/c2ccc(C(F)(F)F)c(OC)c2)C1]

### Synthetic Route:

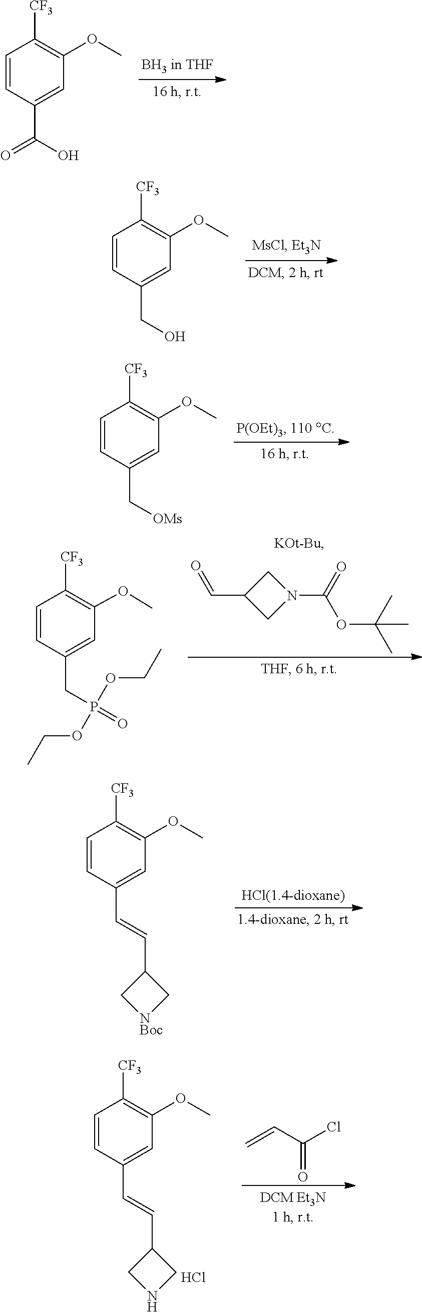

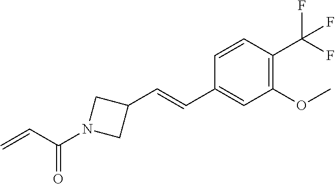 [SMILES: C=CC(=O)N1CC(/C=C/c2ccc(C(F)(F)F)c(OC)c2)C1]

Synthesis of [3-methoxy-4-(trifluoromethyl) phenyl] methanol 3-methoxy-4-(trifluoromethyl) benzoic acid (3 g, 13.627 mmol), a stir bar. BH₃ in THF (40 mL) were added to microwave vial (40 mL×2). The resulting mixture was stirred overnight at r.t. then poured into ice water and extracted with DCM (50 mL×3). The combined layers were washed with brine, dried over anhydrous sodium sulfate, filtered and concentrated under vacuum pressure to give [3-methoxy-4-(trifluoromethyl) phenyl]methanol (2.02 g, crude) as white solid.

### Synthesis of [3-methoxy-4-(trifluoromethyl)phenyl]methyl methanesulfonate

[3-methoxy-4-(trifluoromethyl) phenyl]methanol (1 g, 4.851 mmol), a stir bar, DCM (15 mL) and triethylamine (2.42 g, 23.915 mmol) were added to 50 mL round bottom flask and stirred until homogeneous, then treated with MsCl (850 mg, 7.421 mmol) dropwise at 0° C. The resulting mixture was stirred for 2 h at r.t, diluted with water (10 mL) and extracted with DCM (10 mL×3). The combined layers were washed with brine, dried over anhdrous sodium sulfate, filtered and concentrated under vacuum pressure to give [3-methoxy-4-(trifluoromethyl)phenyl]methyl methanesulfonate (1 g, crude) as yellow oil.

### Synthesis of diethyl [3-methoxy-4-(trifluoromethyl) phenyl]methylphosphonate

[3-methoxy-4-(trifluoromethyl) phenyl]methyl methanesulfonate (1 g, 3.518 mmol), a stir bar, P(OEt)₃ (15 mL) were added to 50 mL round bottom flask. The resulting mixture was stirred overnight at 110° C., cooled to room temperature, and concentrated under vacuum pressure. The residue was purified by reverse-phase chromatography (0%-62% ACN/10 mM NH₄HCO₃ water) to give diethyl [3-methoxy-4-(trifluoromethyl) phenyl]methylphosphonate as green oil. MS (ESI) mass calcd. for C₁₃H₁₈F₃O₄P, 326.09 m/z, found, 327.05 [M+H]⁺.

### Synthesis of tert-butyl 3-[(E)-2-[3-methoxy-4-(trifluoromethyl) phenyl]ethenyl]azetidine-1-carboxylate

Diethyl [3-methoxy-4-(trifluoromethyl) phenyl]methylphosphonate (620 mg, 1.900 mmol), a stir bar, THF (10 mL) and tert-butyl 3-formylazetidine-1-carboxylate (430 mg, 2.322 mmol) were added to 50 mL round bottom flask and stirred until homogeneous, then treated with potassium t-butoxide (323 mg, 2.878 mmol) in batches at 0° C. The resulting mixture was stirred for 6 h at r.t, then diluted with water (10 mL) and extracted with EA (10 mL×3). The combined layers ware washed with brine, dried over anhydrous sodium sulfate, filtered and concentrated under vacuum. The residue was purified by the silica gel chromatography (0%-17% EA in PE) to give tert-butyl 3-[(E)-2-[3-methoxy-4-(trifluoromethyl) phenyl]ethenyl]azetidine-1-carboxylate as green oil. MS (ESI) mass calcd. for C₁₈H₂₂F₃NO₃, 357.16 m/z, found, 302.00 [M-56+H]⁺.

### Synthesis of 3-[(E)-2-[3-methoxy-4-(trifluoromethyl) phenyl]ethenyl]azetidine hydrochloride

Tert-butyl 3-[(E)-2-[3-methoxy-4-(trifluoromethyl) phenyl]ethenyl]azetidine-1-carboxylate (380 mg, 1.063 mmol), a stir bar, 1,4-dioxane (3 mL) were added to 25 mL round bottom flask and stirred until homogeneous, the treated 4 M HCl in 1,4-dioxane (4.00 mL) dropwise at r.t. The resulting mixture was stirred for 2 h at r.t, then concentrated under vacuum to give 3-[(E)-2-[3-methoxy-4-(trifluoromethyl) phenyl]ethenyl]azetidine hydrochloride (320 mg, crude) as white solid. MS (ESI) mass calcd. for C₁₃H₁₄F₃NO, 257.10 m/z, found, 258.00 [M+H]⁺.

### Synthesis of 1-{3-[(E)-2-[3-methoxy-4-(trifluoromethyl) phenyl]ethenyl]azetidin-1-yl}prop-2-en-1-one

3-[(E)-2-[3-methoxy-4-(trifluoromethyl) phenyl]ethenyl]azetidine (150 mg, 0.583 mmol), a stir bar, triethylamine (294 mg, 2.905 mmol) and DCM (3 mL) were added to 25 mL round bottom flask and stirred until homogeneous, then treated with a solution of acryloyl chloride (52.5 mg, 0.593 mmol) in DCM (0.5 mL) dropwise at 0° C. The reaction mixture was stirred d for 1 h at r.t, then diluted with water (5 mL) and extracted with DCM (5 mL×3). The combined organic layers were washed with brine, dried over anhydrous sodium sulfate, filtered and concentrated under vacuum. The residue was purified by Prep-HPLC (Column: XBridge Prep Phenyl OBD Column, 19*250 mm, 5 μm; Mobile Phase A: Water (10 mmol/L NH₄HCO₃+0.1% NH₃·H₂O), Mobile Phase B: ACN; Flow rate: 25 mL/min; Gradient: 38% B to 68% B in 7 min, 68% B; Wave Length: 254 nm; RT1(min): 6) to give 1-{3-[(E)-2-[3-methoxy-4-(trifluoromethyl) phenyl]ethenyl]azetidin-1-yl}prop-2-en-1-one (18.7 mg) as a yellow oil. MS (ESI) mass calcd. for C₁₆H₁₆F₃NO₂, 311.11 m/z, found, 312.05 [M+H]+; ¹H NMR (400 MHz, DMSO-d6) δ 7.55 (d, J=8.1 Hz, 1H), 7.35 (s, 1H), 7.14 (d, J=8.0 Hz, 1H), 6.82 (dd, J=15.9, 8.2 Hz, 1H), 6.58 (d, J=15.9 Hz, 1H), 6.33 (dd, J=16.9, 10.3 Hz, 1H), 6.12 (d, J=16.8, 2.2 Hz, 1H), 5.68 (d, J=10.2, 2.2 Hz, 1H), 4.52-4.44 (m, 1H), 4.23-4.09 (m, 2H), 3.96-3.90 (s, 3H), 3.90-3.82 (m, 1H), 3.56-3.44 (m, 1H). ¹⁹F NMR (376 MHz, DMSO-d₆) δ (ppm): −60.26.

### Example 4: Synthesis of 1-{3-[(E)-2-[3-fluoro-4-(trifluoromethyl)phenyl]ethenyl]azetidin-1-yl}prop-2-en-1-one

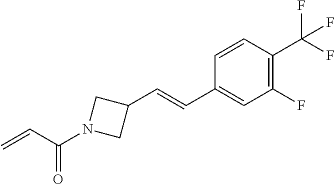 [SMILES: C=CC(=O)N1CC(/C=C/c2ccc(C(F)(F)F)c(F)c2)C1]

### Synthetic Route:

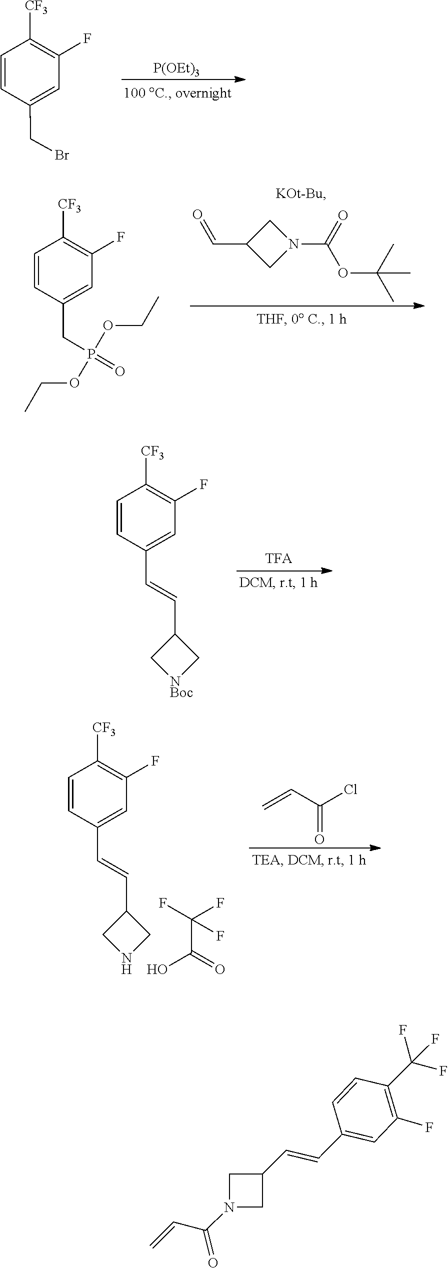

Synthesis of diethyl [3-(trifluoromethyl)phenyl]methylphosphonate 4-(bromomethyl)-2-fluoro-1-(trifluoromethyl)benzene (0.5 g, 1.945 mmol), a stir bar and P(OEt)₃ (5 mL) were added to a 20 mL vial. The resulting mixture was stirred overnight at 100° C., then cooled to r.t. r.t, and concentrated under vacuum. The residue was purified by reverse column chromatography with CH₃CN/10 mM NH₄HCO₃ Water (30%-60%) to afford diethyl [3-(trifluoromethyl)phenyl]methylphosphonate as a light yellow oil (0.575 g, 94.07%), MS (ESI) mass calcd. for C₁₂H₁₈F₄O₃P, 314.2 m/z, found, 315.2 [M+H]⁺.

### Synthesis of tert-butyl 3-[(E)-2-[2-fluoro-4-(trifluoromethyl)phenyl]ethenyl]azetidine-1-carboxylate

Diethyl [2-fluoro-4-(trifluoromethyl)phenyl]methylphosphonate (575 mg, 1.830 mmol), tert-butyl 3-formylazetidine-1-carboxylate (406 mg, 2.192 mmol), a stir bar and THF (10 mL) were added to a 100 mL round-bottom flask and stirred until homogenous, then treated with potassium tert-butoxide (308 mg, 2.745 mmol) at 0° C. The resulting mixture was stirred for 1 h at r.t., then diluted with water (100 mL) and extracted with EA (100 mL×2). The combined extracts were washed with brine, dried over anhydrous sodium sulfate, filtered and concentrated under vacuum. The residue was subjected to silica gel chromatography (0-80% EtOAc/pet ether) to afford tert-butyl 3-[(E)-2-[2-fluoro-4-(trifluoromethyl)phenyl]ethenyl]azetidine-1-carboxylate as a light yellow oil. MS (ESI) mass calcd. for C17H19F4NO2, 345.1 m/z, found, 290.05 [M+H-56]+.

### Synthesis of (E)-3-(3-fluoro-4-(trifluoromethyl)styryl)azetidine 2,2,2-trifluoroacetate

Tert-butyl 3-{2-[5-(trifluoromethyl)-1,2-oxazol-3-yl]ethyl}azetidine-1-carboxylate (200 mg, 0.579 mmol), a stir bar and DCM (10 mL) were added to a 50 mL round-bottom flask, then treated with TFA (2 mL). The resulting mixture was stirred for 1 h at r.t, then concentrated under vacuum to afford a crude product, (E)-3-(3-fluoro-4-(trifluoromethyl)styryl)azetidine 2,2,2-trifluoroacetate as a yellow solid (280 mg). MS (ESI), calcd. for C12H11F4N, 245.1 m/z, found 246.05 [M+H]+.

### Synthesis of 1-{3-[(E)-2-[3-fluoro-4-(trifluoromethyl)phenyl]ethenyl]azetidin-1-yl}prop-2-en-1-one

(E)-3-(3-fluoro-4-(trifluoromethyl)styryl)azetidine 2,2,2-trifluoroacetate (280 mg, 0.779 mmol), triethylamine (410 mg, 4.052 mmol), a stir bar and DCM (8 mL) were added to a 100 mL round-bottom flask and stirred until homogenous, then treated with acryloyl chloride (110 mg, 1.215 mmol) at 0° C. The resulting mixture was stirred at r.t. r.t. for 1 h, then diluted with water (50 mL) and extracted with DCM (50 mL×2). The combined extracts were washed with brine, dried over anhydrous sodium sulfate, filtered and concentrated to dryness to afford crude product, which was further purified by PREP-Chiral-HPLC with (Column: XBridge Prep OBD C18 Column, 30*150 mm, 5 μm; Mobile Phase A: Water (10 mmol/L NH₄HCO₃+0.1% NH₃·H₂O), Mobile Phase B: ACN; Flow rate: 60 mL/min; Gradient: 33% B to 63% B in 7 min, 63% B; Wave Length: 254 nm; RT1(min): 5.87) to afford 1-{3-[(E)-2-[3-fluoro-4-(trifluoromethyl)phenyl]ethenyl]azetidin-1-yl}prop-2-en-1-one as a white solid. MS (ESI) mass calcd. for C₁₅H₁₃F₄NO, 299.10 m/z, found, 300.10[M+H]⁺. ¹H NMR (400 MHz, DMSO-d₆) δ 7.78-7.71 (m, 1H), 7.64 (d, J=12.5 Hz, 1H), 7.49-7.45 (m, 1H), 6.91-6.82 (m, 1H), 6.65-6.58 (m, 1H), 6.37-6.28 (m, 1H), 6.15-6.09 (m, 1H), 5.72-5.65 (m, 1H), 4.54-4.43 (m, 1H), 4.24-4.09 (m, 2H), 3.90-3.82 (m, 1H), 3.57-3.48 (m, 1H). ¹⁹F NMR (376 MHz, DMSO-d6) δ −59.62 −59.69, −116.18 −116.30.

### Example 5: Synthesis of 1-{3-[(E)-2-[3-fluoro-4-(trifluoromethyl)phenyl]ethenyl]azetidin-1-yl}prop-2-en-1-one

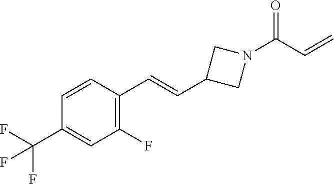 [SMILES: C=CC(=O)N1CC(/C=C/c2ccc(C(F)(F)F)cc2F)C1]

### Synthetic Route:

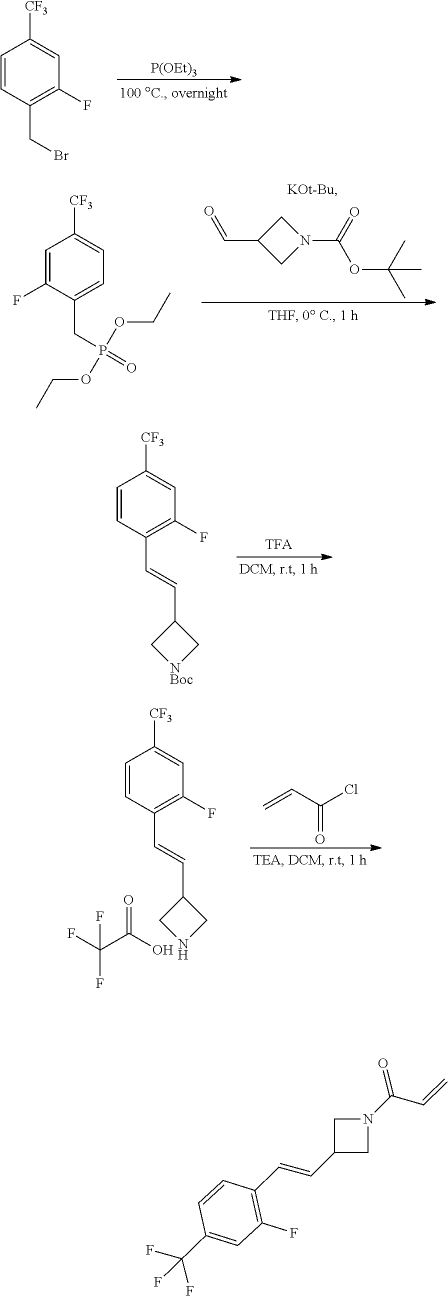

### Synthesis of diethyl [3-(trifluoromethyl)phenyl]methylphosphonate

1-(bromomethyl)-2-fluoro-4-(trifluoromethyl)benzene (500 mg, 1.945 mmol), a stir bar and P(OEt)₃ (5 mL) were added to a 40 mL vial. The resulting mixture was stirred overnight at 100° C., then cooled to r.t, and concentrated under vacuum. The residue was purified by reverse column chromatography with CH₃CN/10 mM NH₄HCO₃ Water (30%-60%) to afford diethyl [3-(trifluoromethyl)phenyl]methylphosphonate as light yellow oil, MS (ESI) mass calcd. for C₁₂H₁₅F₄O₃P, 314.2 m/z, found, 315.2 [M+H]⁺.

### Synthesis of tert-butyl 3-[(E)-2-[2-fluoro-4-(trifluoromethyl)phenyl]ethenyl]azetidine-1-carboxylate

Diethyl [2-fluoro-4-(trifluoromethyl)phenyl]methylphosphonate (537 mg, 1.709 mmol), tert-butyl 3-formylazetidine-1-carboxylate (380 mg, 2.052 mmol), a stir bar and THF (10 mL) were added to a 100 mL round-bottom flask and stirred until homogenous, then treated with potassium tert-butoxide (287 mg, 2.558 mmol) at 0° C. The resulting mixture was stirred for 1 h at r.t, then diluted with water (100 mL) and extracted with EA (100 mL×2). The combined extracts were washed with brine, dried over anhydrous sodium sulfate, filtered and concentrated under vacuum. The residue was subjected to silica gel chromatography (0-80% EtOAc/pet ether) to afford tert-butyl 3-[(E)-2-[2-fluoro-4-(trifluoromethyl)phenyl]ethenyl]azetidine-1-carboxylate as a light yellow oil. MS (ESI) mass calcd. for C₁₇H₁₉F₄NO₂, 345.1 m/z, found, 289.95 [M+H-56]⁺.

### Synthesis of (E)-3-(2-fluoro-4-(trifluoromethyl)styryl)azetidine 2,2,2-trifluoroacetate

Tert-butyl 3-[(E)-2-[2-fluoro-4-(trifluoromethyl)phenyl]ethenyl]azetidine-1-carboxylate (200 mg, 0.579 mmol), a stir bar and DCM (10 mL) were added to a 50 mL round-bottom flask, then treated with TFA (2 mL). The resulting mixture was stirred for 1 h at r.t, then concentrated under vacuum to afford a crude product, (E)-3-(2-fluoro-4-(trifluoromethyl)styryl)azetidine 2,2,2-trifluoroacetate as a yellow solid (250 mg). MS (ESI), calcd. for C₁₂H₁₁F₄N, 245.1 m/z, found 246.45 [M+H]⁺.

### Synthesis of 1-{3-[(E)-2-[3-fluoro-4-(trifluoromethyl)phenyl]ethenyl]azetidin-1-yl}prop-2-en-1-one

(E)-3-(2-fluoro-4-(trifluoromethyl)styryl)azetidine 2,2,2-trifluoroacetate (264 mg, 0.735 mmol), a stir bar, triethylamine (387 mg, 3.824 mmol) and DCM (8 mL) were added to a 100 mL round-bottom flask and stirred until homogenous, then treated with acryloyl chloride (104 mg, 1.149 mmol) at 0° C. The resulting mixture was stirred for 1 h at r.t, then diluted with water (50 mL) and extracted with DCM (50 mL×2). The combined extracts were washed with brine, dried over anhydrous sodium sulfate, filtered and concentrated to dryness to afford crude product, which was further purified by PREP-Chiral-HPLC with (Column: XBridge Prep OBD C18 Column, 30*150 mm, 5 μm; Mobile Phase A: Water (10 mmol/L NH₄HCO₃+0.1% NH₃·H₂O), Mobile Phase B: ACN; Flow rate: 60 mL/mm; Gradient: 36% B to 66% B in 7 min, 66% B; Wave Length: 254 nm; RT1(min): 6) to afford 1-{3-[(E)-2-[3-fluoro-4-(trifluoromethyl)phenyl]ethenyl]azetidin-1-yl}prop-2-en-1-one as an off-white solid (71.5 mg). MS (ESI) mass calcd. for C₁₅H₁₃F₄NO, 299.10 m/z, found, 300.10[M+H]⁺. ¹H NMR (400 MHz, DMSO-d₆) δ 7.93-7.84 (m, 1H), 7.74-7.66 (m, 1H), 7.62-7.55 (m, 1H), 6.90-6.81 (m, 1H), 6.71-6.62 (m, 1H), 6.38-6.26 (m, 1H), 6.16-6.06 (m, 1H), 5.72-5.63 (m, 1H), 4.51-4.43 (m, 1H), 4.22-4.11 (m, 2H), 3.90-3.81 (m, 1H), 3.64-3.51 (m, 1H). ¹⁹F NMR (376 MHz, DMSO-d₆) δ −61.00, −116.43.

### Example 6: Synthesis of 1-{4-[(E)-2-[4-(trifluoromethyl)phenyl]ethenyl]piperidin-1-yl}prop-2-en-1-one

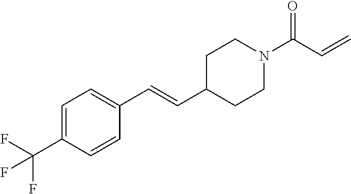 [SMILES: C=CC(=O)N1CCC(/C=C/c2ccc(C(F)(F)F)cc2)CC1]

### Synthetic Route:

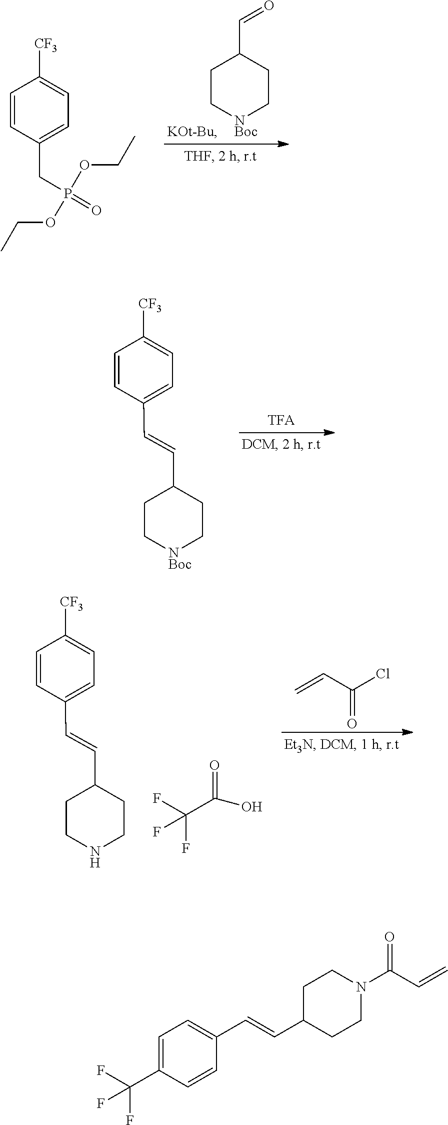

### Synthesis of tert-butyl 4-[(E)-2-[4-(trifluoromethyl)phenyl]ethenyl]piperidine-1-carboxylate

Diethyl [4-(trifluoromethyl)phenyl]methylphosphonate (300 mg, 1.013 mmol), a stir bar, tert-butyl 4-formylpiperidine-1-carboxylate (259 mg, 1.214 mmol), THF (8 mL) were add to an oven-dried 50 mL round-bottom flask, and stirred until homogeneous before the reaction vessel was cooled to 0° C., and charged with potassium t-butoxide (170 mg, 1.515 mmol). The reaction mixture was stirred for 2 h at r.t, and quenched with water extracted with EA (100 mL×2). The combined organic layers were dried over anhydrous sodium sulfate, filtered and concentrated under vacuum. The residue was purified by silica gel chromatography (EA/PE=0~50%) to afford tert-butyl 4-[(E)-2-[4-(trifluoromethyl)phenyl]ethenyl]piperidine-1-carboxylate as a light yellow solid. MS (ESI) mass calcd. for C₁₉H₂₄F₃NO₂, 355.2 m/z, found, 300.05 [M+H-56]⁺

### Synthesis of (E)-4-(4-(trifluoromethyl)styryl)piperidine 2,2,2-trifluoroacetate

Tert-butyl 3-[(E)-2-[4-(trifluoromethyl)phenyl]ethenyl]pyrrolidine-1-carboxylate (270 mg, 0.760 mmol), a stir bar, DCM (10 mL) were added to an oven-dried and nitrogen-purged 50 mL round-bottom flask and stirred until homogeneous before charged with trifluoroacetic acid (2 mL). The resulting mixture was stirred for 2 h at r.t, then concentrated under vacuum to afford (E)-4-(4-(trifluoromethyl)styryl)piperidine 2,2,2-trifluoroacetate (330 mg, crude) as a light yellow semi-solid. MS (ESI) mass calcd. for C₁₄H₁₆F₃N, 255.1 m/z, found, 256.05 [M+H]⁺

### Synthesis of 1-{4-[(E)-2-[4-(trifluoromethyl)phenyl]ethenyl]piperidin-1-yl}prop-2-en-1-one

(E)-4-(4-(trifluoromethyl)styryl)piperidine 2,2,2-trifluoroacetate (330 mg, 0.894 mmol), a stir bar, DCM (6 mL), Et₃N (452 mg, 4.467 mmol) were added to an oven-dried 50 mL round-bottom flask and stirred until homogeneous at 0° C. then treated with a solution of acryloyl chloride (121 mg, 1.337 mmol) in DCM (2 mL). The resulting mixture was stirred for 1 h at room temperature then quenched with water extracted with DCM (100 mL×2). The combined organic layers were dried over anhydrous sodium sulfate, filtered and concentrated under vacuum. The residue was purified by reverse-phase chromatography with ACN/10 mM NH₄HCO₃ water (5~60%) to afford 1-{4-[(E)-2-[4-(trifluoromethyl)phenyl]ethenyl]piperidin-1-yl}prop-2-en-1-one as a yellow solid. MS (ESI) mass calcd. for C₁₇H₁₈F₃NO, 309.15 m/z, found, 310.15 [M+H]⁺; ¹H NMR (300 MHz, DMSO-d6) δ 7.72-7.56 (m, 4H), 6.89-6.75 (m, 1H), 6.50 (s, 2H), 6.14-6.03 (m, 1H), 5.71-5.61 (m, 1H), 4.51-4.37 (m, 1H), 4.14-4.01 (m, 1H), 3.21-3.05 (m, 1H), 2.83-2.65 (m, 1H), 2.45-2.35 (m, 1H), 1.87-1.72 (m, 2H), 1.39-1.17 (m, 2H). ¹⁹F NMR (376 MHz, DMSO-d₆) δ (ppm): −60.79.

### Example 7: Synthesis of (E)-1-(3-fluoro-3-(2-(5-(trifluoromethyl)isoxazol-3-yl)vinyl)azetidin-1-yl)prop-2-en-1-one

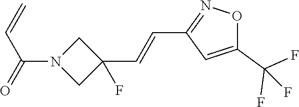 [SMILES: C=CC(=O)N1CC(F)(/C=C/c2cc(C(F)(F)F)on2)C1]

### Synthetic Route:

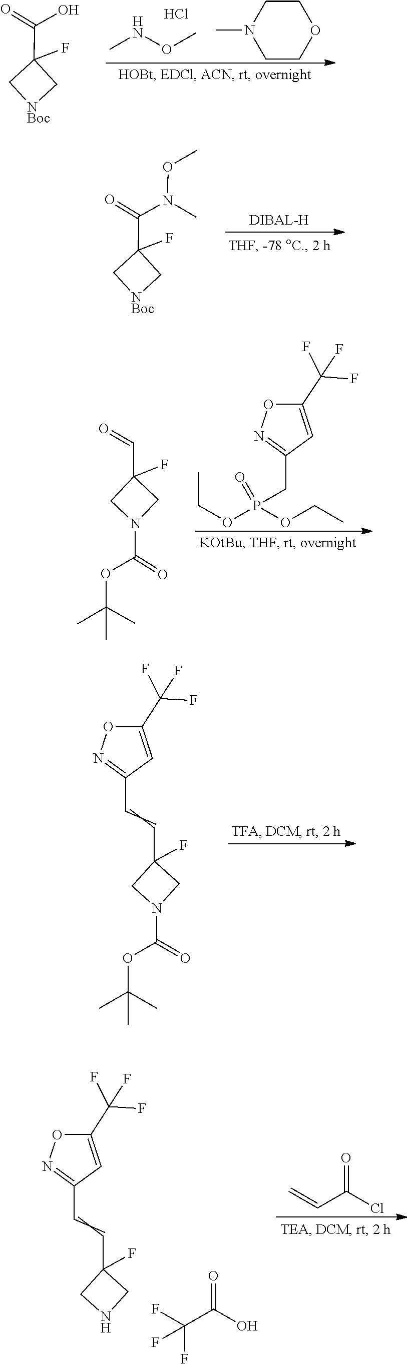

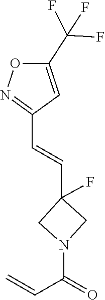 [SMILES: C=CC(=O)N1CC(F)(/C=C/c2cc(C(F)(F)F)on2)C1]

### Synthesis of tert-butyl 3-fluoro-3-(methoxy(methyl)carbamoyl)azetidine-1-carboxylate

4-[4-(trifluoromethyl)phenoxy]benzoic acid (1 g, 3.543 mmol), 1-(tert-butoxycarbonyl)-3-fluoroazetidine-3-carboxylic acid (2 g, 9.124 mmol). N,O-dimethylhydroxylamine hydrochloride (1.8 g, 18.454 mmol), a stir bar and ACN (20 mL) were added to a 100 mL round-bottom flask and stirred until homogenous, then treated with HOBt (2.5 g, 18.501 mmol), EDCI (3 g, 15.649 mmol) and N-methylmorpholine (6.3 mL, 11.864 mmol) in batches at rt. The resulting mixture was stirred overnight at 25° C., then diluted with water. The resulting mixture was extracted with DCM (20 mL×3). The combined organic layers were washed with brine, dried over anhydrous sodium sulfate, filtered and concentrated under vacuum. The residue was purified by silica gel column (0% to 50% EA in PE) to afford tert-butyl 3-fluoro-3-[methoxy(methyl)carbamoyl]azetidine-1-carboxylate (750 mg, 31.34%) as a white oil. ¹H NMR (300 MHz, DMSO-d₆) δ 4.40 (d, J=11.2 Hz, 1H), 4.32 (d, J=11.0 Hz, 1H), 4.06 (d, J=11.0 Hz, 1H), 3.98 (d, J=11.0 Hz, 1H), 3.70 (s, 3H), 3.18 (d, J=1.6 Hz, 3H), 1.39 (s, 9H).

### Synthesis of tert-butyl 3-fluoro-3-formylazetidine-1-carboxylate

Tert-butyl 3-fluoro-3-[methoxy(methyl)carbamoyl]azetidine-1-carboxylate (750 mg, 2.860 mmol). THF (2 mL, 24.686 mmol) and a stir bar were added to a 20 mL vial and stirred until homogenous, then treated with DIBAL-H (4 mL, 19.716 mmol) dropwise at −78° C. The resulting mixture was stirred for 2 h at −78° C., then quenched with water, filtered and washed with DCM (20 mL). The resulting mixture was extracted with DCM (5 mL×3). The combined organic layers were washed with brine, dried over anhydrous sodium sulfate, filtered and concentrated under vacuum. to afford tert-butyl 3-fluoro-3-formylazetidine-1-carboxylate (700 mg, 120.46%) as yellow oil.

### Synthesis of tert-butyl 3-fluoro-3-(2-(5-(trifluoromethyl)isoxazol-3-yl)vinyl)azetidine-1-carboxylate

Tert-butyl 3-fluoro-3-formylazetidine-1-carboxylate (700 mg, 3.445 mmol), diethyl [5-(trifluoromethyl)-1,2-oxazol-3-yl]methylphosphonate (112 mg, 0.390 mmol), a stir bar and THF (7 mL) were added to a 20 mL vial and stirred until homogenous, then treated with potassium tert-butoxide (482 mg, 4.295 mmol) in batches at rt. The resulting mixture was stirred overnight at 25° C., then quenched with water. The resulting mixture was extracted with DCM (10 mL×3). The combined organic layers were washed with brine, dried over anhydrous sodium sulfate, filtered and concentrated under vacuum. The residue was purified by silica gel chromatography (0% to 40% EA in PE) to afford tert-butyl 3-fluoro-3-{2-[5-(trifluoromethyl)-1,2-oxazol-3-yl]ethenyl}azetidine-1-carboxylate (280 mg, 24.17%) as a white solid. MS (ESI) mass calcd. for C₁₄H₁₆F₄N₂O₃, 336.00 m/z, found, 322.05 [M+H+CH₃CN]⁺.

### Synthesis of 3-(2-(3-fluoroazetidin-3-yl)vinyl)-5-(trifluoromethyl)isoxazole 2,2,2-trifluoroacetate

Tert-butyl 3-fluoro-3-{2-[5-(trifluoromethyl)-1,2-oxazol-3-yl]ethenyl}azetidine-1-carboxylate (280 mg, 0.833 mmol), DCM (2.5 mL) and a stir bar were added to an 8 mL vial and stirred until homogenous, then treated with TFA (0.5 mL, 6.732 mmol) in dropwise at rt. The resulting mixture was stirred for 2 h at 25° C., concentrated under vacuum to afford 3-(2-(3-fluoroazetidin-3-yl)vinyl)-5-(trifluoromethyl)isoxazole 2,2,2-trifluoroacetate (400 mg, 203.42% crude) as a yellow oil. MS (ESI) calcd. for C₉H₈F₄N₂O, 236.00 m/z, found 237.00 [M+H]⁺.

### Synthesis of (E)-1-(3-fluoro-3-(2-(5-(trifluoromethyl)isoxazol-3-yl)vinyl)azetidin-1-yl)prop-2-en-1-one

3-(2-(3-fluoroazetidin-3-yl)vinyl)-5-(trifluoromethyl)isoxazole 2,2,2-trifluoroacetate (378 mg, 1.386 mmol), TEA (808 mg, 7.985 mmol), a stir bar and DCM (4 mL) were added to a 100 mL round-bottom flask and stirred until homogenous, then treated with acryloyl chloride (216 mg, 2.386 mmol) dropwise at rt. The resulting mixture was stirred overnight at 25° C., then quenched with water. The resulting mixture was extracted with DCM (20 mL×3). The combined organic layers were washed with brine, dried over anhydrous sodium sulfate, filtered and concentrated under vacuum. Then the resulting mixture was purified by Prep-HPLC (Column: Column: XBridge Prep OBD C18 Column, 30*150 mm, 5 μm; Mobile Phase A: Water (10 mmol/L NH₄HCO₃+0.1% NH₃·H₂O). Mobile Phase B: ACN; Flow rate: 60 mL/min; Gradient: 37% B to 67% B in 7 min, 67% B; Wave Length: 254 nm; RT1(min): 6) to afford 1-{3-fluoro-3-[(E)-2-[5-(trifluoromethyl)-1,2-oxazol-3-yl]ethenyl]azetidin-1-yl}prop-2-en-1-one (53.5 mg,) as a white solid. MS (ESI) calcd. for C₁₂H₁₀F₄N₂O₂, 290.07 m/z, found 290.95 [M+H]¹; ¹H NMR (300 MHz, DMSO-d₆) δ 7.83 (s, 1H), 7.28-7.09 (m, 1H), 6.96-6.83 (m, 1H), 6.43-6.28 (m, 1H), 6.23-6.10 (m, 1H), 5.80-5.68 (m, 1H), 4.70-4.48 (m, 2H), 4.40-4.14 (m, 2H); ¹⁹F NMR (282 MHz, DMSO-d₆) δ −63.31, −152.45.

### Example 8: Synthesis of (Z)-1-(3-fluoro-3-(2-(5-(trifluoromethyl)isoxazol-3-yl)vinyl)azetidin-1-yl)prop-2-en-1-one

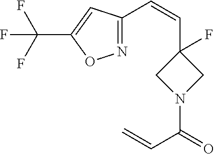 [SMILES: C=CC(=O)N1CC(F)(/C=C\c2cc(C(F)(F)F)on2)C1]

### Synthetic Route:

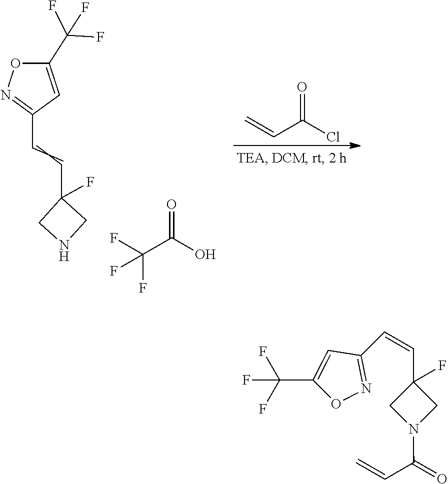

### Synthesis of (Z)-1-(3-fluoro-3-(2-(5-(trifluoromethyl)isoxazol-3-yl)vinyl)azetidin-1-yl)prop-2-en-1-one

3-(2-(3-fluoroazetidin-3-yl)vinyl)-5-(trifluoromethyl)isoxazole 2,2,2-trifluoroacetate (378 mg, 1.386 mmol), TEA (808 mg, 7.985 mmol), a stir bar and DCM (4 mL) were added to a 100 mL round-bottom flask and stirred until homogenous, then treated with acryloyl chloride (216 mg, 2.386 mmol) dropwise at rt. The resulting mixture was stirred overnight at 25° C., then quenched with water. The resulting mixture was extracted with DCM (20 mL×3). The combined organic layers were washed with brine, dried over anhydrous sodium sulfate, filtered and concentrated under vacuum. Then the resulting mixture was purified by Prep-HPLC (Column: Column: XBridge Prep OBD C18 Column, 30*150 mm, 5 μm; Mobile Phase A: Water (10 mmol/L NH₄HCO₃+0.1% NH₃·H₂O), Mobile Phase B: ACN; Flow rate: 60 mL/min; Gradient: 37% B to 67% B in 7 min, 67% B; Wave Length: 254 nm; RT1(min): 6) to afford And 1-{3-fluoro-3-[(Z)-2-[5-(trifluoromethyl)-1,2-oxazol-3-yl]ethenyl]azetidin-1-yl}prop-2-en-1-one (4.9 mg, 1.22%) as a yellow oil. MS (ESI) calcd. for C₁₂H₁₀F₄N₂O₂, 290.07 m/z, found 291.00 [M+H]⁺; ¹H NMR (300 MHz, DMSO-d₆) δ 7.46 (s, 1H), 6.78-6.68 (m, 1H), 6.67-6.51 (m, 1H), 6.39-6.24 (m, 1H), 6.19-6.07 (m, 1H), 6.76-6.66 (m, 1H), 4.64-4.52 (m, 2H), 4.29 (s, 1H), 4.22 (s, 1H); ¹⁹F NMR (282 MHz, DMSO-d₆) δ −62.96, −143.89.

### Example 9: Synthesis of 1-[(3S)-3-{2-[4-(trifluoromethyl)phenyl]ethenyl}pyrrolidin-1-yl]prop-2-en-1-one

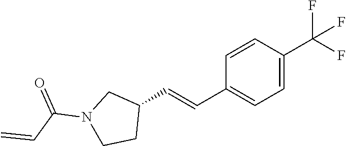 [SMILES: C=CC(=O)N1CC[C@@H](/C=C/c2ccc(C(F)(F)F)cc2)C1]

### Synthetic Route:

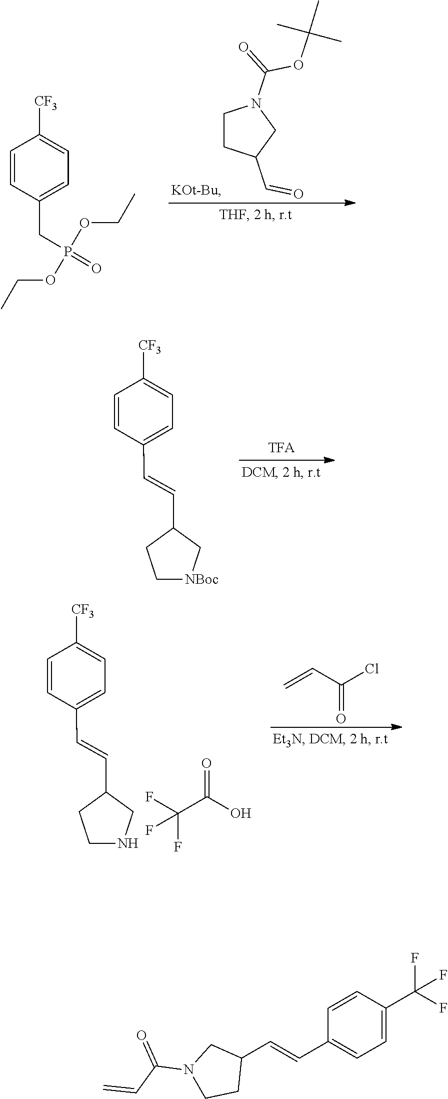

### (Followed by SFC Separation)

### Synthesis of tert-butyl 3-[(E)-2-[4-(trifluoromethyl)phenyl]ethenyl]pyrrolidine-1-carboxylate

Diethyl [4-(trifluoromethyl)phenyl]methylphosphonate (300 mg, 1.013 mmol), a stir bar, tert-butyl 3-formylpyrrolidine-1-carboxylate (242 mg, 1.215 mmol), THF (8 mL) were add to an oven-dried 50 mL round-bottom flask, and stirred until homogeneous before the reaction vessel was cooled to 0° C., and charged with potassium t-butoxide (170 mg, 1.515 mmol). The reaction mixture was stirred for 2 h at r.t, and quenched with water extracted with EA (100 mL×2). The combined organic layers were dried over anhydrous sodium sulfate, filtered and concentrated under vacuum. The residue was purified by silica gel chromatography (EA/PE=0-30%) to afford tert-butyl 3-[(E)-2-[4-(trifluoromethyl)phenyl]ethenyl]pyrrolidine-1-carboxylate as a light yellow oil. MS (ESI) mass calcd. for C₁₈H₂₂F₃NO₂, 341.2 m/z, found, 286.00 [M+H-56]⁺

### Synthesis of (E)-3-(4-(trifluoromethyl)styryl)pyrrolidine 2,2,2-trifluoroacetate

Tert-butyl 3-[(E)-2-[4-(trifluoromethyl)phenyl]ethenyl]pyrrolidine-1-carboxylate (150 mg, 0.439 mmol), a stir bar, DCM (10 mL) were added to an oven-dried and nitrogen-purged 50 mL round-bottom flask, and stirred until homogeneous before charged with trifluoroacetic acid (2 mL). The resulting mixture was stirred for 2 h at r.t., then concentrated under vacuum to afford (E)-3-(4-(trifluoromethyl)styryl)pyrrolidine 2,2,2-trifluoroacetate as a light brown semi-solid. MS (ESI) mass calcd. for C₁₃H₁₄F₃N, 241.1 m/z, found, 242.05 [M+H]⁺

### Synthesis of 1-{3-[(E)-2-[4-(trifluoromethyl)phenyl]ethenyl]pyrrolidin-1-yl}prop-2-en-1-one

(E)-3-(4-(trifluoromethyl)styryl)pyrrolidine 2,2,2-trifluoroacetate (154 mg, 0.433 mmol), a stir bar, DCM (6 mL), Et₃N (219 mg, 2.164 mmol) were add to an oven-dried and nitrogen-purged 50 mL round-bottomed flask, and the resulting mixture stirred until homogeneous before the reaction vessel was cooled to 0° C., and charged with a solution of acryloyl chloride (59 mg, 0.652 mmol) in DCM (2 mL). The resulting mixture was stirred for 2 h at r.t, and quenched with water extracted with DCM (50 mL×2). The combined organic layers were dried over anhydrous sodium sulfate, filtered and concentrated under vacuum. The residue was purified by reverse-phase chromatography ACN/10 mM NH₄HCO₃ water (5%-55%) to afford 1-{3-[(E)-2-[4-(trifluoromethyl)phenyl]ethenyl]pyrrolidin-1-yl}prop-2-en-1-one as alight yellow solid product. MS (ESI) mass calcd. for C₁₆H₁₆F₃NO, 295.10 m/z, found, 296.10 [M+H]⁺; ¹H NMR (400 MHz, DMSO-d6) δ 7.74-7.62 (m, 4H), 6.69-6.45 (m, 3H), 6.18-6.09 (m, 1H), 5.74-5.62 (m, 1H), 3.89-3.51 (m, 3H), 3.23-2.96 (m, 2H), 2.18-2.01 (m, 1H), 1.95-1.69 (m, 1H), ¹⁹F NMR (376 MHz, DMSO-d₆) δ (ppm): −60.83.

### Synthesis of 1-[(3S)-3-{2-[4-(trifluoromethyl)phenyl]ethenyl}pyrrolidin-1-yl]prop-2-en-1-one

The racemic product was further separated by SFC (Column: CHIRAL ART Cellulose-SB, 2*25 cm, 5 μm; Mobile Phase A: Hex (0.5% 2M NH₃-MeOH)—HPLC, Mobile Phase B: IPA—HPLC; Flow rate: 20 mL/min; Gradient: 15% B to 15% B in 21 min; Wave Length: 220/254 nim; RT1(min): 14.491 (first isomer); Sample Solvent: EtOH—HPLC; Injection Volume: 0.4 mL; Number Of Runs: 2) to afford one stereoisomer, assigned as (S) (first eluting isomer), 1-[(3S)-3-{2-[4-(trifluoromethyl)phenyl]ethenyl}pyrrolidin-1-yl]prop-2-en-1-one as a white solid (4.7 mg). MS (ESI) mass calcd. for C₁₆H₁₆F₃NO, 295.10 m/z, found, 296.15 [M+H]⁺). ¹H NMR (400 MHz, DMSO-d6) δ 7.74-7.62 (m, 4H), 6.69-6.45 (m, 3H), 6.18-6.09 (m, 1H), 5.74-5.62 (m, 1H), 3.89-3.51 (m, 3H), 3.23-2.96 (m, 2H), 2.18-2.01 (m, 1H), 1.95-1.69 (m, 1H). ¹⁹F NMR (376 MHz, DMSO-d₆) δ (ppm): −60.82.

### Example 10: Synthesis of 1-[(3R)-3-{2-[4-(trifluoromethyl)phenyl]ethenyl}pyrrolidin-1-yl]prop-2-en-1-one

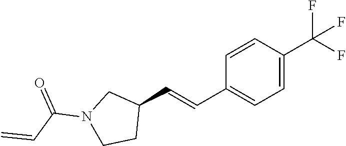 [SMILES: C=CC(=O)N1CC[C@H](/C=C/c2ccc(C(F)(F)F)cc2)C1]

### Synthesis of 1-[(3R)-3-{2-[4-(trifluoromethyl)phenyl]ethenyl}pyrrolidin-1-yl]prop-2-en-1-one

The racemic product was separated by SFC (Column: CHIRAL ART Cellulose-SB, 2*25 cm, 5 μm; Mobile Phase A: Hex (0.5% 2M NH₃-MeOH)—HPLC. Mobile Phase B: IPA—HPLC; Flow rate: 20 mL/min; Gradient: 15% B to 15% B in 21 min; Wave Length: 220/254 nm; RT2(min): 18.48 (second isomer); Sample Solvent: EtOH—HPLC; Injection Volume: 0.4 mL; Number Of Runs: 2) to afford one stereoisomer, assigned as R (second eluting isomer), 1-[(3R)-3-{2-[4-(trifluoromethyl)phenyl]ethenyl}pyrrolidin-1-yl]prop-2-en-1-one as a white solid (3.8 mg). MS (ESI) mass calcd. for C₁₆H₁₆F₃NO, 295.10 m/z, found, 296.10 [M+H]⁺). ¹H NMR (400 MHz, DMSO-d6) δ 7.74-7.62 (m, 4H), 6.69-6.45 (m, 3H), 6.18-6.09 (m, 1H), 5.74-5.62 (m, 1H), 3.89-3.51 (m, 3H), 3.23-2.96 (m, 2H), 2.18-2.01 (m, 1H), 1.95-1.69 (m, 1H). ¹⁹F NMR (376 MHz, DMSO-d₆) δ (ppm): −60.82.

### Example 11: Synthesis of 1-(3-(2-(5-(trifluoromethyl)pyridin-2-yl)vinyl)azetidin-1-yl)prop-2-en-1-one

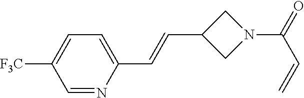 [SMILES: C=CC(=O)N1CC(/C=C/c2ccc(C(F)(F)F)cn2)C1]

### Synthetic Route:

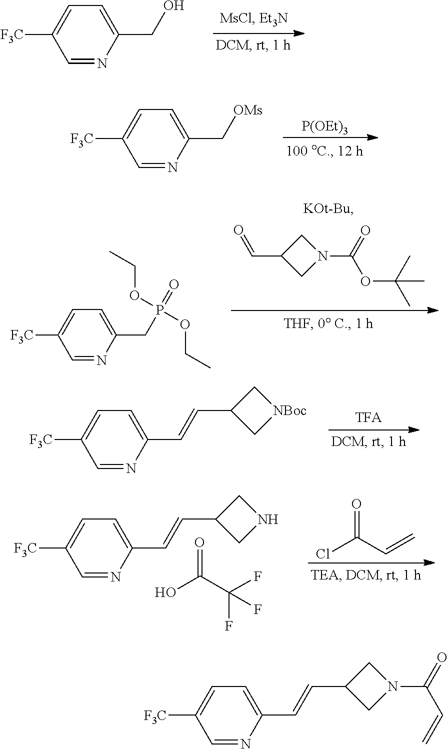

### Synthesis of (5-(trifluoromethyl)pyridin-2-yl)methyl methanesulfonate

(5-(trifluoromethyl)pyridin-2-yl)methanol (1.0 g, 5.646 mmol), a stir bar. Et₃N (1.14 g, 11.292 mmol), DCM (20 mL) were added to a 100 mL round-bottom flask and stirred until homogeneous, then treated with MsCl (0.78 g, 6.810 mmol) dropwise. The mixture was stirred at r.t. for 1 h, then quenched with water (50 mL) and extracted with DCM (100 mL×3). The combined organic extracts were washed with brine, dried over anhydrous Na₂SO₄, filtered and concentrated to dryness under reduced pressure. The residue was purified on a silica gel column eluted with EA/PE (0-50%) to afford (5-(trifluoromethyl)pyridin-2-yl)methyl methanesulfonate as a light yellow solid. MS (ESI) mass calcd. for C₈H₈F₃NO₃S, 255.02 m/z, found, 255.90 [M+H]⁺.

### Synthesis of diethyl ((5-(trifluoromethyl)pyridin-2-yl)methyl)phosphonate

The solution of (5-(trifluoromethyl)pyridin-2-yl)methyl methanesulfonate (0.98 g, 3.840 mmol) in toluene (10 mL) and triethyl phosphite (3 mL) was heated to 110° C., and stirred overnight, then cooled to r.t, and concentrated under vacuum. The residue was purified by reverse column chromatography with CH₃CN/10 mM NH₄HCO₃ Water (5%-60%) to afford diethyl ((5-(trifluoromethyl)pyridin-2-yl)methyl)phosphonate as a light brown liquid. MS (ESI) mass calcd. for C₁₁H₁₅F₃NO₃P, 297.07 m/z, found, 298.00 [M+H]⁺.

### Synthesis of tert-butyl 3-(2-(5-(trifluoromethyl)pyridin-2-yl)vinyl)azetidine-1-carboxylate

Diethyl ((5-(trifluoromethyl)pyridin-2-yl)methyl)phosphonate (470 mg, 1.581 mmol), tert-butyl 3-formylazetidine-1-carboxylate (360 mg, 1.944 mmol), a stir bar, and THF (8 mL) were added to a 50 mL round-bottom flask and stirred until homogeneous, and then treated with potassium tert-butoxide (266 mg, 2.370 mmol) at 0° C. The reaction mixture was stirred for 2 h at rt, then diluted with water (30 mL), and extracted with EtOAc (50 mL×2). The combined extracts were dried over anhydrous sodium sulfate, filtered, and concentrated to dryness. The residue was purified on a silica gel column eluted with EA/PE (0-50%) to afford tert-butyl 3-(2-(5-(trifluoromethyl)pyridin-2-yl)vinyl)azetidine-1-carboxylate as a light yellow oil. MS (ESI) mass calcd. for C₁₆H₁₉F₃N₂O₂, 328.14 m/z, found, 273.15 [M+H-56]⁺.

### Synthesis of 2-(2-(azetidin-3-yl)vinyl)-5-(trifluoromethyl)pyridine 2,2,2-trifluoroacetate

Tert-butyl 3-(2-(5-(trifluoromethyl)pyridin-2-yl)vinyl)azetidine-1-carboxylate (410 mg, 1.249 mmol), a stir bar, and DCM (10 mL) were added to a 50 mL round-bottom flask and stirred until homogeneous, and then treated with TFA (2 mL). The reaction mixture was stirred for 2 h at r.t., then concentrated under vacuum to afford 2-(2-(azetidin-3-yl)vinyl)-5-(trifluoromethyl)pyridine 2,2,2-trifluoroacetate as a light brown oil (600 mg, crude). MS (ESI) mass calcd. for C₁₁H₁₁F₃N₂, 228.09 m/z, found, 229.10 [M+H]⁺.

### Synthesis of 1-(3-(2-(5-(trifluoromethyl)pyridin-2-yl)vinyl)azetidin-1-yl)prop-2-en-1-one

2-(2-(azetidin-3-yl)vinyl)-5-(trifluoromethyl)pyridine 2,2,2-trifluoroacetate (600 mg, 1.753 mmol), a stir bar, Et₃N (888 mg, 8.775 mmol), DCM (13 mL) were added to a 50 mL round-bottom flask and stirred until homogeneous, then treated with a solution of acryloyl chloride (206 mg, 2.276 mmol) in DCM (2 mL) dropwise. The mixture was stirred at r.t. for 1 h, then quenched with water (30 mL) and extracted with DCM (50 mL×3). The combined organic extracts were washed with brine, dried over anhydrous Na₂SO₄, filtered and concentrated under vacuum. The residue was purified by HPLC with (Column: XBridge Prep OBD C18 Column, 30*150 mm, 5 μm; Mobile Phase A: Water (10 mmol/L NH₄HCO₃+0.1% NH₃·H₂O), Mobile Phase B: ACN; Flow rate: 60 mL/min; Gradient: 30% B to 60% B in 7 min, 60% B; Wave Length: 254 nm; RT1(min): 6; Number Of Runs: 0) to afford 1-(3-(2-(5-(trifluoromethyl)pyridin-2-yl)vinyl)azetidin-1-yl)prop-2-en-1-one as a light orange solid (121.1 mg). MS (ESI) mass calcd. for C₁₄H₁₃F₃N₂O, 282.10 m/z, found, 283.00 [M+H]⁺, ¹H NMR (300 MHz, DMSO-d6) δ 8.92-8.86 (m, 1H), 8.21-8.13 (m, 1H), 7.68 (d, J=8.3 Hz, 1H), 7.18 (dd, J=15.7, 8.2 Hz, 1H), 6.73 (dd, J=15.7, 1.1 Hz, 1H), 6.40-6.27 (m, 1H), 6.17-6.06 (m, 1H), 5.68 (dd, J=10.3, 2.4 Hz, 1H), 4.52-4.43 (m, 1H), 4.23-4.11 (m, 2H), 3.92-3.82 (m, 1H), 3.67-3.51 (m, 1H). ¹⁹F NMR (282 MHz, DMSO-d₆) δ −60.68.

### Example 12: Synthesis of 1-{3-[(Z)-2-[2-(trifluoromethyl)-1,3-thiazol-5-yl]ethenyl]azetidin-1-yl}prop-2-en-1-one

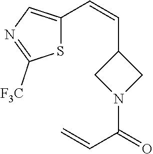 [SMILES: C=CC(=O)N1CC(/C=C\c2cnc(C(F)(F)F)s2)C1]

### Synthetic Route:

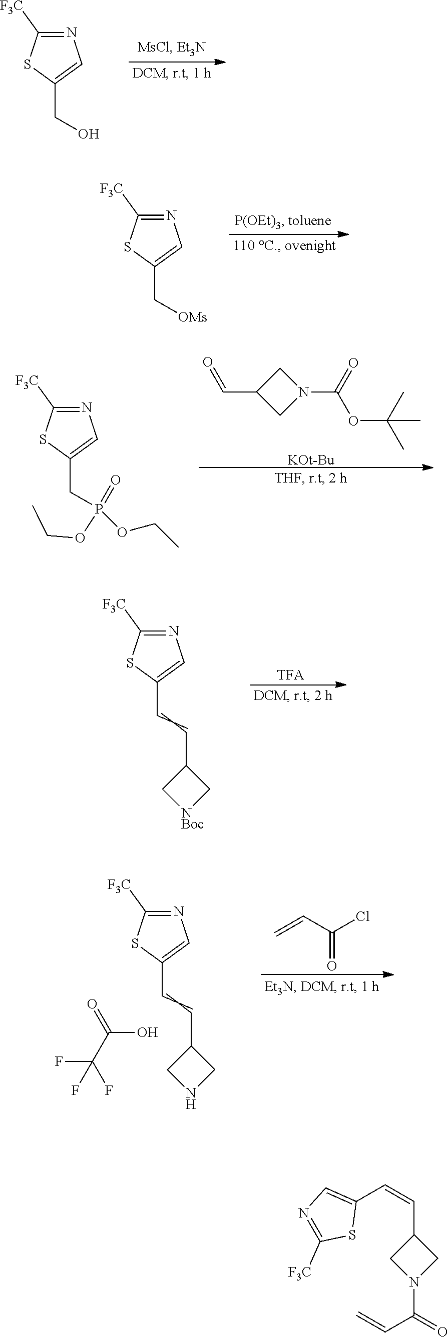

### Synthesis of methyl [2-(trifluoromethyl)-1,3-thiazol-5-yl]methyl methanesulfonate

[2-(trifluoromethyl)-1,3-thiazol-5-yl]methanol (1.0 g, 5.460 mmol), a stir bar, Et₃N (1.10 g, 10.865 mmol), DCM (20 mL) were added to a 100 mL round-bottom flask and stirred until homogeneous, then treated with MsCl (0.74 g, 6.443 mmol) dropwise. The mixture was stirred at r.t. for 1 h, then quenched with water (50 mL) and extracted with DCM (100 mL×3). The combined organic extracts were washed with brine, dried over anhydrous Na₂SO₄, filtered and concentrated to dryness under reduced pressure. The residue was purified on a silica gel column eluted with EA/PE (0-50%) to afford [2-(trifluoromethyl)-1,3-thiazol-5-yl]methyl methanesulfonate as a light yellow oil. MS (ESI) mass calcd. for C₆H₆F₃NO₃S₂, 260.97 m/z, found, 261.95 [M+H]⁺.

### Synthesis of diethyl [2-(trifluoromethyl)-1,3-thiazol-5-yl]methylphosphonate

The solution of [2-(trifluoromethyl)-1,3-thiazol-5-yl]methyl methanesulfonate (0.75 g, 2.871 mmol) in toluene (10 mL) and triethyl phosphite (3 mL) was heated to 110° C., and stirred overnight, then cooled to r.t, and concentrated under vacuum. The residue was purified by reverse column chromatography with CH₃CN/10 mM NH₄HCO₃ Water (5%-60%) to afford diethyl [2-(trifluoromethyl)-1,3-thiazol-5-yl]methylphosphonate as a light brown liquid. MS (ESI) mass calcd. for C₉H₁₃F₃NO₃PS, 303.03 m/z, found, 304.00 [M+H]⁺.

### Synthesis of tert-butyl 3-{2-[2-(trifluoromethyl)-1,3-thiazol-5-yl]ethenyl}azetidine-1-carboxylate

Diethyl [2-(trifluoromethyl)-1,3-thiazol-5-yl]methylphosphonate (400 mg, 1.319 mmol), tert-butyl 3-formylazetidine-1-carboxylate (293 mg, 1.582 mmol), a stir bar, and THF (10 mL) were added to a 50 mL round-bottom and stirred until homogeneous, and then treated with potassium tert-butoxide (222 mg, 1.978 mmol) at 0° C. The reaction mixture was stirred for 2 h at r.t, then diluted with water (30 mL), and extracted with EtOAc (50 mL×2). The combined extracts were dried over anhdrous sodium sulfate, filtered, and concentrated to dryness. The residue was purified on a silica gel column eluted with EA/PE (0-50%) to afford tert-butyl 3-{2-[2-(trifluoromethyl)-1,3-thiazol-5-yl]ethenyl}azetidine-1-carboxylate as a light yellow oil. MS (ESI) mass calcd. for C₁₄H₁₇F₃N₂O₂S, 334.36 m/z, found, 278.95 [M+H-56]⁺.

### Synthesis of 5-[2-(azetidin-3-yl)ethenyl]-2-(trifluoromethyl)-1,3-thiazole 2,2,2-trifluoroacetate

Tert-butyl 3-{2-[2-(trifluoromethyl)-1,3-thiazol-5-yl]ethenyl}azetidine-1-carboxylate (300 mg, 0.897 mmol), a stir bar, and DCM (10 mL) were added to a 50 mL round-bottom flask and stirred until homogeneous, and then treated with TFA (2 mL). The reaction mixture was stirred for 2 h at r.t., then concentrated under vacuum to afford 5-[2-(azetidin-3-yl)ethenyl]-2-(trifluoromethyl)-1,3-thiazole 2,2,2-trifluoroacetate as a light brown oil (400 mg, crude). MS (ESI) mass calcd. for C₉H₉F₃N₂S, 234.04 m/z, found, 235.00 [M+H]⁺.

### Synthesis of 1-{3-[(Z)-2-[2-(trifluoromethyl)-1,3-thiazol-5-yl]ethenyl]azetidin-1-yl}prop-2-en-1-one

5-[2-(azetidin-3-yl)ethenyl]-2-(trifluoromethyl)-1,3-thiazole 2,2,2-trifluoroacetate (400 mg, 1.149 mmol), a stir bar. Et₃N (596 mg, 5.890 mmol), DCM (10 mL) were added to a 50 mL round-bottom flask and stirred until homogeneous, then treated with a solution of acryloyl chloride (156 mg, 1.724 mmol) in DCM (2 mL) dropwise. The mixture was stirred at r.t. for 1 h, then quenched with water (30 mL) and extracted with DCM (50 mL×3). The combined organic extracts were washed with brine, dried over anhydrous Na₂SO₄, filtered and concentrated under vacuum. The residue was purified by HPLC with (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 25% B to 55% B in 7 min, 55% B; Wave Length: 254 nm; RT1(min): 6.10) to afford a mixture which was further separated by Achiral SFC with (Column: YMC-Actus SIL, 3*25 cm, 5 μm; Mobile Phase A: CO₂, Mobile Phase B: IPA (0.5% 2M NH₃-MeOH); Flow rate: 50 mL/min; Gradient: isocratic 13% B; Column Temperature (° C.); 35; Back Pressure (bar); 100; Wave Length: 254 nm; RT1(min): 5.43; Sample Solvent: MeOH—HPLC; Injection Volume: 1 mL; Number Of Runs: 13) to afford 1-{3-[(Z)-2-[2-(trifluoromethyl)-1,3-thiazol-5-yl]ethenyl]azetidin-1-yl}prop-2-en-1-one as a colorless oil (27.8 mg). MS (ESI) mass calcd. for C₁₂H₁₁F₃N₂OS, 288.05 m/z, found, 289.05 [M+H]⁺, ¹H NMR (400 MHz, DMSO-d₆) δ 8.12 (s, 1H), 6.80 (d, J=11.4 Hz, 1H), 6.36-6.26 (m, 2H), 6.11 (dd, J=17.0, 2.3 Hz, 1H), 5.67 (dd, J=10.2, 2.3 Hz, 1H), 4.63-4.55 (m, 1H), 4.33-4.25 (m, 1H), 4.09-4.02 (m, 1H), 3.85-3.72 (m, 2H) ¹⁹F NMR (282 MHz, DMSO-d₆) δ (ppm): −59.81).

### Example 13: Synthesis of 1-{3-[(E)-2-[2-(trifluoromethyl)-1,3-thiazol-5-yl]ethenyl]azetidin-1-yl}prop-2-en-1-one

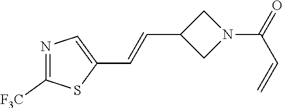 [SMILES: C=CC(=O)N1CC(/C=C/c2cnc(C(F)(F)F)s2)C1]

### Synthetic Route:

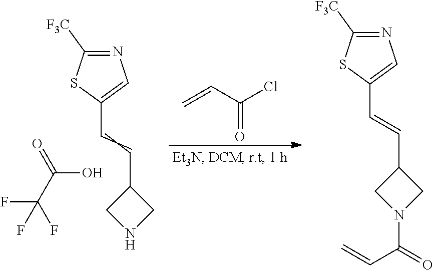

### Synthesis of 1-{3-[(E)-2-[2-(trifluoromethyl)-1,3-thiazol-5-yl]ethenyl]azetidin-1-yl}prop-2-en-1-one

5-[2-(azetidin-3-yl)ethenyl]-2-(trifluoromethyl)-1,3-thiazole 2,2,2-trifluoroacetate (400 mg, 1.149 mmol), a stir bar, Et₃N (596 mg, 5.890 mmol), DCM (10 mL) were added to a 50 mL round-bottom flask and stirred until homogeneous, then treated with a solution of acryloyl chloride (156 mg, 1.724 mmol) in DCM (2 mL) dropwise. The mixture was stirred at r.t. for 1 h, then quenched with water (30 mL) and extracted with DCM (50 mL×3). The combined organic extracts were washed with brine, dried over anhydrous Na₂SO₄, filtered and concentrated under vacuum. The residue was purified by HPLC with (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 25% B to 55% B in 7 min, 55% B; Wave Length: 254 nm; RT1(min): 6.10) to afford a mixture which was further separated by Achiral SFC with (Column: YMC-Actus SIL, 3*25 cm, 5 μm; Mobile Phase A: CO₂, Mobile Phase B: IPA (0.5% 2M NH₃-MeOH); Flow rate: 50 mL/min; Gradient: isocratic 13% B; Column Temperature (° C.); 35; Back Pressure (bar); 100; Wave Length: 254 nm; RT2(min): 6.37; Sample Solvent: MeOH—HPLC; Injection Volume: 1 mL; Number Of Runs: 13) to afford 1-{3-[(E)-2-[2-(trifluoromethyl)-1,3-thiazol-5-yl]ethenyl]azetidin-1-yl}prop-2-en-1-one as a white solid (85.0 mg), MS (ESI) mass calcd. for C₁₂H₁₁F₃N₂OS, 288.05 m/z, found, 289.05 [M+H]⁺, ¹H NMR (400 MHz, DMSO-d₆) δ 8.08 (s, 1H), 6.86 (d, J=15.8 Hz, 1H), 6.66 (dd, J=15.8, 8.0 Hz, 1H), 6.31 (dd, J=17.0, 10.3 Hz, 1H), 6.10 (dd, J=17.0, 2.3 Hz, 1H), 5.67 (dd, J=10.3, 2.3 Hz, 1H), 4.50-4.41 (m, 1H), 4.21-4.07 (m, 2H), 3.89-3.80 (m, 1H), 3.59-3.45 (m, 1H) ¹⁹F NMR (282 MHz, DMSO-d₆) δ (ppm): −60.00).

### Example 14: Synthesis of 1-(3-(3-(trifluoromethyl)styryl)azetidin-1-yl)prop-2-en-1-one

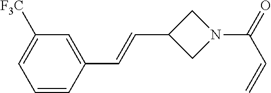 [SMILES: C=CC(=O)N1CC(/C=C/c2cccc(C(F)(F)F)c2)C1]

### Synthetic Route:

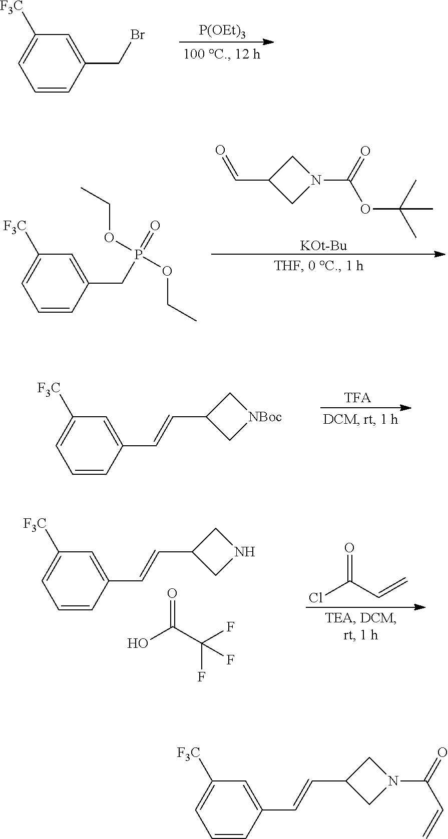

### Synthesis of diethyl (3-(trifluoromethyl)benzyl)phosphonate

The mixture of 1-(bromomethyl)-3-(trifluoromethyl)benzene (1.0 g, 4.183 mmol) and triethyl phosphite (10 mL) was heated to 110° C., and stirred overnight, then cooled to r.t. and concentrated under vacuum. The residue was purified by reverse column chromatography with CH₃CN/10 mM NH₄HCO₃ Water (5%-60%) to afford diethyl (3-(trifluoromethyl)benzyl)phosphonate as a light brown liquid. MS (ESI) mass calcd. for C₁₂H₁₆F₃O₃P, 296.08 m/z, found, 397.00 [M+H]⁺.

### Synthesis of tert-butyl 3-(3-(trifluoromethyl)styryl)azetidine-1-carboxylate

Diethyl (3-(trifluoromethyl)benzyl)phosphonate (500 mg, 1.688 mmol), a stir bar, and THF (10 mL) were added to a 50 mL round-bottom and stirred until homogeneous, and then treated with potassium tert-butoxide (285 mg, 2.540 mmol). After the mixture was stirred for 20 min at rt, tert-butyl 3-formylazetidine-1-carboxylate (375 mg, 2.025 mmol) was added. The reaction mixture was stirred for 2 h at rt, then diluted with water (30 mL), and extracted with EtOAc (50 mL×2). The combined extracts were dried over anhydrous sodium sulfate, filtered, and concentrated to dryness. The residue was purified on a silica gel column eluted with EA/PE (0-20%) to afford tert-butyl 3-(3-(trifluoromethyl)styryl)azetidine-1-carboxylate as a light yellow oil. MS (ESI) mass calcd. for C₁₇H₂₀F₃NO₂, 327.14 m/z, found, 272.00 [M+H-56]⁺.

### Synthesis of 3-(3-(trifluoromethyl)styryl)azetidine 2,2,2-trifluoroacetate

Tert-butyl 3-(3-(trifluoromethyl)styryl)azetidine-1-carboxylate (120 mg, 0.367 mmol), a stir bar, and DCM (5 mL) were added to a 50 mL round-bottom flask and stirred until homogeneous, and then treated with TFA (1 mL). The reaction mixture was stirred for 2 h at r.t., then concentrated under vacuum to afford 3-(3-(trifluoromethyl)styryl)azetidine 2,2,2-trifluoroacetate as a light brown oil (130 mg, crude). MS (ESI) mass calcd. for C₁₂H₁₂F₃N, 227.09 m/z, found, 228.10 [M+H]⁺.

### Synthesis of 1-(3-(3-(trifluoromethyl)styryl)azetidin-1-yl)prop-2-en-1-one

3-(3-(trifluoromethyl)styryl)azetidine 2,2,2-trifluoroacetate (130 mg, 0.381 mmol), a stir bar, Et₃N (193 mg, 1.907 mmol), DCM (5 mL) were added to a 50 mL round-bottom flask and stirred until homogeneous, then treated with a solution of acryloyl chloride (52 mg, 0.575 mmol) in DCM (1 mL) dropwise. The mixture was stirred at r.t. for 1 h, then quenched with water (30 mL) and extracted with DCM (50 mL×3). The combined organic extracts were washed with brine, dried over anhydrous Na₂SO₄, filtered and concentrated under vacuum. The residue was purified by HPLC with (Column: XSelect CSH Prep C18 OBD Column, 19*250 mm, 5 μm; Mobile Phase A: Water (10 mmol/L NH₄HCO₃+0.1% NH₃·H₂O), Mobile Phase B: ACN; Flow rate: 25 mL/min; Gradient: 42% B to 72% B in 7 min, 72% B; Wave Length: 254 nm; RT1(min): 6) to afford 1-(3-(3-(trifluoromethyl)styryl)azetidin-1-yl)prop-2-en-1-one as a light yellow oil (32.5 mg). MS (ESI) mass calcd. for C₁₅H₁₄F₃NO, 281.10 m/z, found, 282.15 [M+H]⁺; ¹H NMR (400 MHz, DMSO-d6) δ 7.82 (s, 1H), 7.79-7.72 (m, 1H), 7.64-7.50 (m, 2H), 6.83-6.72 (m, 1H), 6.68-6.58 (m, 1H), 6.38-6.28 (m, 1H), 6.17-6.06 (m, 1H), 5.68 (dd, J=10.3, 2.3 Hz, 1H), 4.47 (t, J=8.5 Hz, 1H), 4.21-4.09 (m, 2H), 3.90-3.81 (m, 1H), 3.56-3.44 (m, 1H). ¹⁹F NMR (376 MHz, DMSO-d₆) δ −61.16.

### Example 15: Synthesis of 1-{3-[(E)-2-[4-(trifluoromethyl)phenyl]ethenyl]azetidin-1-yl}prop-2-en-1-one

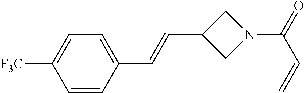 [SMILES: C=CC(=O)N1CC(/C=C/c2ccc(C(F)(F)F)cc2)C1]

### Synthetic Route:

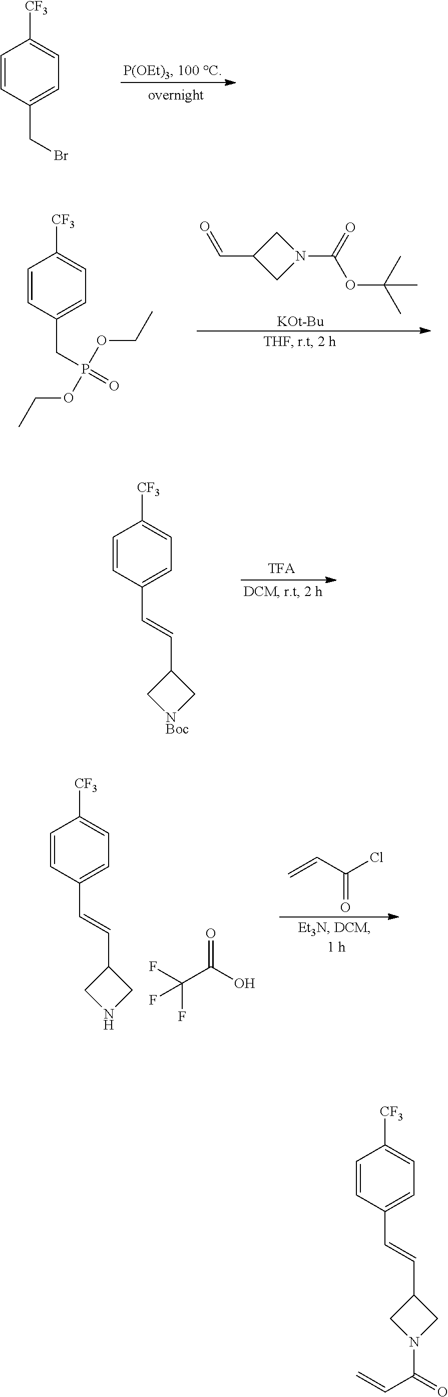

### Synthesis of diethyl [4-(trifluoromethyl)phenyl]methylphosphonate

The mixture of 1-(bromomethyl)-4-(trifluoromethyl)benzene (1.0 g, 4.183 mmol) and triethyl phosphite (10 mL) was heated to 110° C., and stirred overnight, then cooled to r.t. and concentrated under vacuum. The residue was purified by reverse column chromatography with CH₃CN/10 mM NH₄HCO₃ Water (5%-60%) to afford diethyl [4-(trifluoromethyl)phenyl]methylphosphonate as a light brown liquid. MS (ESI) mass calcd. for C₁₂H₁₆F3O₃P, 296.08 m/z, found, 397.00 [M+H]⁺.

### Synthesis of tert-butyl 3-[(E)-2-[4-(trifluoromethyl)phenyl]ethenyl]azetidine-1-carboxylate

Diethyl [4-(trifluoromethyl)phenyl]methylphosphonate (500 mg, 1.688 mmol), a stir bar, and THF (10 mL) were added to a 50 mL round-bottom and stirred until homogeneous, and then treated with potassium tert-butoxide (285 mg, 2.540 mmol). After the mixture was stirred for 20 min at r.t, tert-butyl 3-formylazetidine-1-carboxylate (375 mg, 2.025 mmol) was added. The reaction mixture was stirred for 2 h at r.t, then diluted with water (30 mL), and extracted with EtOAc (50 mL×2). The combined extracts were dried over anhydrous sodium sulfate, filtered, and concentrated to dryness. The residue was purified on a silica gel column eluted with EA/PE (0-20%) to afford tert-butyl 3-[(E)-2-[4-(trifluoromethyl)phenyl]ethenyl]azetidine-1-carboxylate as a light yellow semi-solid. MS (ESI) mass calcd. for C₁₇H₂₀F₃NO₂, 327.14 m/z, found, 272.05 [M+H-56]⁺.

### Synthesis of 3-[(E)-2-[4-(trifluoromethyl)phenyl]ethenyl]azetidine 2,2,2-trifluoroacetate

Tert-butyl 3-[(E)-2-[4-(trifluoromethyl)phenyl]ethenyl]azetidine-1-carboxylate (300 mg, 0.916 mmol), a stir bar, and DCM (10 mL) were added to a 50 mL round-bottom flask and stirred until homogeneous, and then treated with TFA (2 mL). The reaction mixture was stirred for 2 h at r.t., then concentrated under vacuum to afford 3-[(E)-2-[4-(trifluoromethyl)phenyl]ethenyl]azetidine 2,2,2-trifluoroacetate as light brown oil (340 mg, crude). MS (ESI) mass calcd. for C₁₂H₁₂F₃N, 227.09 m/z, found, 228.10 [M+H]⁺.

### Synthesis of 1-{3-[(E)-2-[4-(trifluoromethyl)phenyl]ethenyl]azetidin-1-yl}prop-2-en-1-one

3-[(E)-2-[4-(trifluoromethyl)phenyl]ethenyl]azetidine 2,2,2-trifluoroacetate (340 mg, 0.996 mmol), a stir bar, Et3N (505 mg, 4.990 mmol), DCM (7 mL) were added to a 50 mL round-bottom flask and stirred until homogeneous, then treated with a solution of acryloyl chloride (135 mg, 1.492 mmol) in DCM (1 mL) dropwise. The mixture was stirred at r.t. for 1 h, then quenched with water (30 mL) and extracted with DCM (50 mL×3). The combined organic extracts were washed with brine, dried over anhydrous Na₂SO₄, filtered and concentrated under vacuum. The residue was purified by HPLC with (Column: XSelect CSH Prep C18 OBD Column, 19*250 mm, 5 μm; Mobile Phase A: Water (10 mmol/L NH₄HCO₃+0.1% NH₃·H₂O), Mobile Phase B: ACN; Flow rate: 25 mL/min; Gradient: 42% B to 72% B in 7 min, 72% B; Wave Length: 254 nm) to afford 1-{3-[(E)-2-[4-(trifluoromethyl)phenyl]ethenyl]azetidin-1-yl}prop-2-en-1-one as a light yellow solid (69.5 mg). MS (ESI) mass calcd. for C₁₅H₁₄F₃NO, 281.10 m/z, found, 282.00 [M+H]⁺; ¹H NMR (300 MHz, DMSO-d6) δ 7.77-7.59 (m, 4H), 6.83-6.70 (m, 1H), 6.68-6.55 (m, 1H), 6.40-6.25 (m, 1H), 6.18-6.03 (m, 1H), 5.68 (dd, J=10.2, 2.4 Hz, 1H), 4.52-4.41 (m, 1H), 4.24-4.06 (m, 2H), 3.91-3.78 (m, 1H), 3.61-3.42 (m, 1H), ¹⁹F NMR (282 MHz, DMSO-d6) δ (ppm): −60.85

### Example 16: Synthesis of 1-(3-methyl-3-(2-(5-(trifluoromethyl)isoxazol-3-yl)vinyl)azetidin-1-yl)prop-2-en-1-one

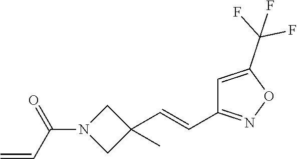 [SMILES: C=CC(=O)N1CC(C)(/C=C/c2cc(C(F)(F)F)on2)C1]

### Synthetic Route:

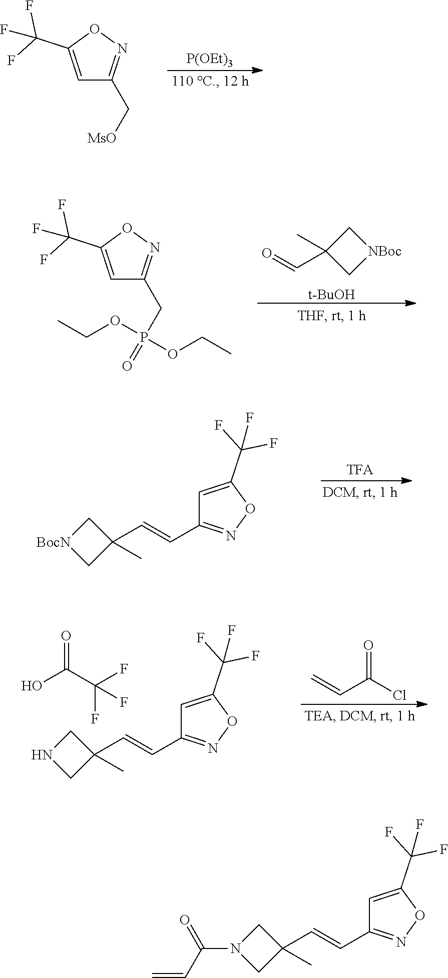

### Synthesis of 3-((diethoxy((methylene)-λ⁵-phosphaneyl)methyl)-5-(trifluoromethyl)isoxazole

(5-(trifluoromethyl)isoxazol-3-yl)methyl methanesulfonate (800 mg, 3.263 mmol), a stir bar, P(OEt)₃ (10 mL) were added to a 50 mL round-bottom flask and stirred until homogeneous. The resulting mixture was stirred over night at 110° C. The reaction mixture was then subjected to reverse phase chromatography on C18 (ACN/H₂O (0.05% NH₄HCO₃)=5~50%) to afford 3-((diethoxy(methylene)-λ⁵-phosphaneyl)methyl)-5-(trifluoromethyl)isoxazole as a light yellow oil (735 g, 78.44%). MS (ESI) calcd. for C₉H₁₃F₃NO₄P, 287.05 m/z, found 288.05 [M+H]⁺.

### Synthesis of tert-butyl 3-methyl-3-(2-(5-(trifluoromethyl)isoxazol-3-yl)vinyl)azetidine-1-carboxylate

3-((diethoxy(methylene)-λ⁵-phosphaneyl)methyl)-5-(trifluoromethyl)isoxazole (300 mg, 1.052 mmol), a stir bar, tert-butyl 3-formyl-3-methylazetidine-1-carboxylate (314 mg, 1.576 mmol) and THF (6 mL) were added to a 50 mL round-bottom flask and stirred until homogeneous, and then treated with t-BuOK (177 mg, 1.577 mmol) at 0° C. The resulting mixture was stirred for 1 h at room temperature. The reaction mixture was then quenched with water (10 mL), extracted with EA (3×20 mL), and the combined extracts were dried over anhydrous Na₂SO₄, filtered, and concentrated to dryness in vacuo. The residue obtained was then subjected to silica gel chromatography (20-50% EA/PE) to afford tert-butyl 3-methyl-3-(2-(5-(trifluoromethyl)isoxazol-3-yl)vinyl)azetidine-1-carboxylate as a white solid (283 mg, 80.96%. MS (ESI) calcd. for C₁₅H₁₉F₃N₂O₃, 332.13 m/z, found 276.95 [M+H-56]⁺).

### Synthesis of 3-(2-(3-methylazetidin-3-yl)vinyl)-5-(trifluoromethyl)isoxazole 2,2,2-trifluoroacetate

Tert-butyl 3-methyl-3-(2-(5-(trifluoromethyl)isoxazol-3-yl)vinyl)azetidine-1-carboxylate (283 mg, 0.852 mmol), a stir bar, DCM (9 mL) were added to a 50 mL round-bottom flask and stirred until homogeneous, and then treated with TFA (3 mL). The reaction mixture was stirred for 2 h at room temperature, then concentrated under vacuum to afford 3-(2-(3-methylazetidin-3-yl)vinyl)-5-(trifluoromethyl)isoxazole 2,2,2-trifluoroacetate as a red oil (402 mg, crude, MS (ESI) mass calcd. for C₁₀H₁₁F₃N₂O, 232.08 m/z, found, 233.10 [M+H]⁺).

### Synthesis of 1-(3-methyl-3-(2-(5-(trifluoromethyl)isoxazol-3-yl)vinyl)azetidin-1-yl)prop-2-en-1-one

3-(2-(3-methylazetidin-3-yl)vinyl)-5-(trifluoromethyl)isoxazole 2,2,2-trifluoroacetate (402 mg, 1.161 mmol), a stir bar, TEA (588 mg, 5.811 mmol) and DCM (8 mL) were added to a 50 mL round-bottom flask and stirred until homogeneous, and then treated with acryloyl chloride (210 mg, 2.320 mmol). The mixture was stirred for 1 h at room temperature, then quenched with water (20 mL) and extracted with DCM (3×30 mL). The combined organic extracts were washed with brine, dried over anhydrous Na₂SO₄, filtered and concentrated under vacuum. The residue was purified by HPLC with (Column: XBridge Prep OBD C18 Column, 30*150 mm, 5 μm; Mobile Phase A: Water (10 mmol/L NH₄HCO₃+0.1% NH₃·H₂O), Mobile Phase B: ACN; Flow rate: 60 mL/min; Gradient: 45% B to 75% B in 7 min, 75% B; Wave Length: 254 nm) to afford 1-(3-methyl-3-(2-(5-(trifluoromethyl)isoxazol-3-yl)vinyl)azetidin-1-yl)prop-2-en-1-one as a light yellow solid (62.2 mg). MS (ESI) mass calcd. for C₁₃H₁₃F₃N₂O₂, 286.09 m/z, found, 287.00 [M+H]⁺. ¹H NMR (400 MHz, DMSO-d₆) δ 7.90-7.79 (m, 1H), 7.19-7.07 (m, 1H), 6.70-6.58 (m, 1H), 6.40-6.27 (m, 1H), 6.20-6.07 (m, 1H), 5.76-5.65 (m, 1H), 4.37-4.21 (m, 1H), 4.16-4.05 (m, 1H), 4.02-3.92 (m, 1H), 3.85-3.74 (m, 1H), 1.48 (s, 3H). ¹⁹F NMR (376 MHz, DMSO-d₆) δ −63.33].

### Example 17: Synthesis of 1-(3-methoxy-3-(4-(trifluoromethyl)styryl)azetidin-1-yl)prop-2-en-1-one

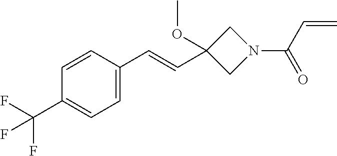 [SMILES: C=CC(=O)N1CC(/C=C/c2ccc(C(F)(F)F)cc2)(OC)C1]

### Synthetic Route:

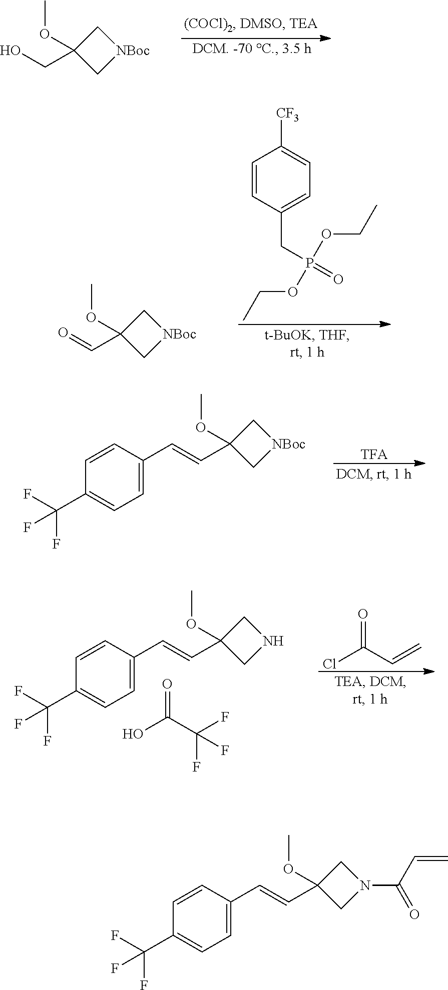

### Synthesis of tert-butyl 3-formyl-3-methoxyazetidine-1-carboxylate

To a solution of (COCl)₂ (1.1 g, 8.657 mmol) in DCM (5 mL) was added dropwise a solution of DMSO (1.3 g, 16.651 mmol) in DCM (4 mL) at −70° C. under nitrogen atmosphere. After stirring for 30 min, tert-butyl 3-(hydroxymethyl)-3-methoxyazetidine-1-carboxylate (0.9 g, 4.142 mmol) in DCM (5 mL) was added dropwise and stirring continued for 30 min. To the mixture was added Et₃N (3.4 g, 33.592 mmol) in DCM (4 mL) and the resulting mixture was stirred at −70° C. for 10 min, then warmed to room temperature and stirred for 1 h. The reaction mixture was then quenched with water (20 mL), extracted with DCM (3×40 mL). The combined organic layers were washed with brine, dried over anhydrous Na₂SO₄, filtered, and concentrated under vacuum to afford tert-butyl 3-formyl-3-methoxyazetidine-1-carboxylate as a dark yellow oil (787 mg, 88.26%, MS (ESI) calcd. for C₁₀H₁₇NO₄, 215.11 m/z, found 177.95 [M+H-56+18]⁺).

### Synthesis of tert-butyl 3-methoxy-3-(4-(trifluoromethyl)styryl)azetidine-1-carboxylate

Diethyl (4-(trifluoromethyl)benzyl)phosphonate (400 mg, 1.350 mmol), a stir bar, tert-butyl 3-formyl-3-methoxyazetidine-1-carboxylate (349 mg, 1.621 mmol) and THF (8 mL) were added to a 50 mL round-bottom flask and stirred until homogeneous, and then treated with t-BuOK (227 mg, 2.023 mmol) at 0° C. The resulting mixture was stirred for 1 h at room temperature. The reaction mixture was then quenched with water (10 mL), extracted with EA (3×20 mL), and the combined extracts dried over anhydrous Na₂SO₄, filtered, and concentrated to dryness in vacuo. The residue obtained was then subjected to silica gel chromatography (0-30% EA/PE) to afford tert-butyl 3-methoxy-3-(4-(trifluoromethyl)styryl)azetidine-1-carboxylate as a light yellow oil (449 mg, 93.04%, MS (ESI) calcd. for C₁₈H₂₂F₃NO₃, 357.16 m/z, found 302.10 [M+H-56]⁺).

### Synthesis of 3-methoxy-3-(4-(trifluoromethyl)styryl)azetidine 2,2,2-trifluoroacetate

Tert-butyl 3-methoxy-3-(4-(trifluoromethyl)styryl)azetidine-1-carboxylate (200 mg, 0.560 mmol), a stir bar, DCM (6 mL) were added to a 50 mL round-bottom flask and stirred until homogeneous, and then treated with TFA (2 mL). The reaction mixture was stirred for 2 h at room temperature, then concentrated under vacuum to afford 3-methoxy-3-(4-(trifluoromethyl)styryl)azetidine 2,2,2-trifluoroacetate as a colorless oil (239 mg crude). MS (ESI) mass calcd. for C₁₃H₁₄F₃NO, 257.10 m/z, found, 258.15 [M+H]⁺).

### Synthesis of 1-(3-methoxy-3-(4-(trifluoromethyl)styryl)azetidin-1-yl)prop-2-en-1-one

3-methoxy-3-(4-(trifluoromethyl)styryl)azetidine 2,2,2-trifluoroacetate (239 mg, 0.644 mmol), a stir bar, TEA (326 mg, 3.222 mmol) and DCM (5 mL) were added to a 50 mL round-bottom flask and stirred until homogeneous, and then treated with acryloyl chloride (117 mg, 1.293 mmol). The mixture was stirred for 1 h at room temperature, then quenched with water (20 mL) and extracted with DCM (3×30 mL). The combined organic extracts were washed with brine, dried over anhydrous Na₂SO₄, filtered and concentrated under vacuum. The residue was purified by HPLC with (Column: XBridge Prep OBD C18 Column, 30*150 mm, 5 μm; Mobile Phase A: Water (10 mmol/L NH₄HCO₃+0.1% NH₃·H₂O), Mobile Phase B: ACN; Flow rate: 60 mL/min; Gradient: 35% B to 65% B in 7 min, 65% B; Wave Length: 254 nm; RT1(min): 6) to afford 1-(3-methoxy-3-(4-(trifluoromethyl)styryl)azetidin-1-yl)prop-2-en-1-one as an off-white solid (79.5 mg). MS (ESI) mass calcd. for C₁₆H₁₆F₃NO₂, 311.11 m/z, found, 312.05 [M+H]⁺, 1H NMR (400 MHz, DMSO-d₆) δ 7.80-7.62 (m, 4H), 6.92-6.84 (m, 1H), 6.72-6.63 (m, 1H), 6.42-6.30 (m, 1H), 6.18-6.10 (m, 1H), 5.75-5.67 (m, 1H), 4.43-4.34 (m, 1H), 4.33-4.26 (m, 1H), 4.15-4.06 (m, 1H), 4.03-3.94 (m, 1H), 3.21 (s, 3H). ¹⁹F NMR (376 MHz, DMSO-d₆) δ −60.93].

### Example 18: Synthesis of 1-(3-methyl-3-(4-(trifluoromethyl)styryl)azetidin-1-yl)prop-2-en-1-one

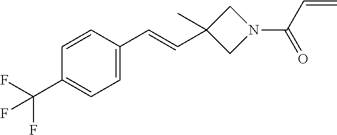 [SMILES: C=CC(=O)N1CC(C)(/C=C/c2ccc(C(F)(F)F)cc2)C1]

### Synthetic Route:

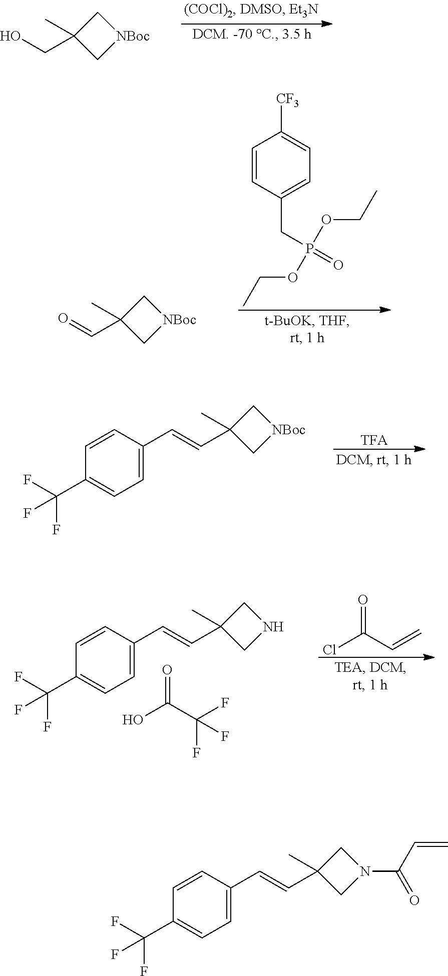

### Synthesis of tert-butyl 3-formyl-3-methylazetidine-1-carboxylate

To a solution of (COCl)₂ (1.0 g, 10.243 mmol) in DCM (5 mL) was added dropwise a solution of DMSO (1.6 g, 20.479 mmol) in DCM (5 mL) at −70° C. under nitrogen atmosphere. After stirring for 30 min, tert-butyl 3-(hydroxymethyl)-3-methoxyazetidine-1-carboxylate (1.0 g, 4.969 mmol) in DCM (5 mL) was added dropwise and stirring continued for 30 min. To the mixture was added Et₃N (4.0 g, 39.528 mmol) in DCM (5 mL) and the resulting mixture was stirred at −70° C. for 10 min, then warmed to room temperature and stirred for 1 h. The reaction mixture was then quenched with water (20 mL), extracted with DCM (3×40 mL). The combined organic layers were washed with brine, dried over anhydrous Na₂SO₄, filtered, and concentrated under vacuum to afford tert-butyl 3-formyl-3-methylazetidine-1-carboxylate as a dark yellow oil (972 mg, 98.18%). MS (ESI) calcd. for C₁₀H₁₇NO₃, 199.12 m/z, found 218.05 [M+H+18]⁺.

### Synthesis of tert-butyl 3-methyl-3-(4-(trifluoromethyl)styryl)azetidine-1-carboxylate

Diethyl (4-(trifluoromethyl)benzyl)phosphonate (500 mg, 1.688 mmol), a stir bar, tert-butyl 3-formyl-3-methylazetidine-1-carboxylate (404 mg, 2.028 mmol) and THF (10 mL) were added to a 50 mL round-bottom flask and stirred until homogeneous, and then treated with t-BuOK (284 mg, 2.531 mmol) at 0° C. The resulting mixture was stirred for 1 h at room temperature. The reaction mixture was then quenched with water (10 mL), extracted with EA (3×20 mL), and the combined extracts dried over anhydrous Na₂SO₄, filtered, and concentrated to dryness in vacuo. The residue obtained was then subjected to silica gel chromatography (0-30% EA/PE) to afford tert-butyl 3-methyl-3-(4-(trifluoromethyl)styryl)azetidine-1-carboxylate as a colorless oil (412 mg, 71.50%). MS (ESI) calcd. for C₁₈H₂₂F₃NO₂, 341.16 m/z, found 286.00 [M+H-56]⁺.

### Synthesis of 3-methyl-3-(4-(trifluoromethyl)styryl)azetidine 2,2,2-trifluoroacetate

Tert-butyl 3-methyl-3-(4-(trifluoromethyl)styryl)azetidine-1-carboxylate (200 mg, 0.586 mmol), a stir bar, DCM (6 mL) were added to a 50 mL round-bottom flask and stirred until homogeneous, and then treated with TFA (2 mL). The reaction mixture was stirred for 2 h at room temperature, then concentrated under vacuum to afford 3-methyl-3-(4-(trifluoromethyl)styryl)azetidine 2,2,2-trifluoroacetate as a light yellow oil (423 mg, crude). MS (ESI) mass calcd. for C₁₃H₁₄F₃N, 241.11 m/z, found, 242.10 [M+H]⁺.

### Synthesis of 1-(3-methyl-3-(4-(trifluoromethyl)styryl)azetidin-1-yl)prop-2-en-1-one

3-methyl-3-(4-(trifluoromethyl)styryl)azetidine 2,2,2-trifluoroacetate (423 mg, 1.191 mmol), a stir bar, TEA (602 mg, 5.949 mmol) and DCM (8 mL) were added to a 50 mL round-bottom flask and stirred until homogeneous, and then treated with acryloyl chloride (216 mg, 2.386 mmol). The mixture was stirred for 1 h at room temperature, then quenched with water (20 mL) and extracted with DCM (3×30 mL). The combined organic extracts were washed with brine, dried over anhydrous Na₂SO₄, filtered and concentrated under vacuum. The residue was purified by HPLC with (Column: XBridge Shield RP18 OBD Column, 30*150 mm, 5 μm; Mobile Phase A: Water (10 mmol/L NH₄HCO₃), Mobile Phase B: ACN; Flow rate: 60 mL/min; Gradient: 34% B to 64% B in 7 min, 64% B; Wave Length: 254 nm; RT1(min): 5.72) to afford 1-(3-methyl-3-(4-(trifluoromethyl)styryl)azetidin-1-yl)prop-2-en-1-one as a light yellow oil (38.6 mg). MS (ESI) mass calcd. for C₁₆H₁₆F₃NO, 295.12 m/z, found, 296.05 [M+H]⁺, ¹H NMR (400 MHz, DMSO-d₆) δ 7.72-7.61 (m, 4H), 6.86-6.78 (m, 1H), 6.65-6.57 (m, 1H), 6.37-6.28 (m, 1H), 6.15-6.06 (m, 1H), 5.68 (dd, J=10.3, 2.3 Hz, 1H), 4.28-4.22 (m, 1H), 4.08-4.02 (m, 1H), 4.01-3.94 (m, 1H), 3.79-3.71 (m, 1H), 1.48 (s, 3H). ¹⁹F NMR (376 MHz, DMSO-d₆) δ −60.83.

### Example 19: Synthesis of 1-(3-(2-methoxy-4-(trifluoromethyl)styryl)azetidin-1-yl)prop-2-en-1-one

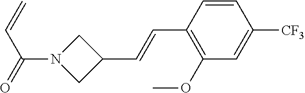 [SMILES: C=CC(=O)N1CC(/C=C/c2ccc(C(F)(F)F)cc2OC)C1]

### Synthetic Route:

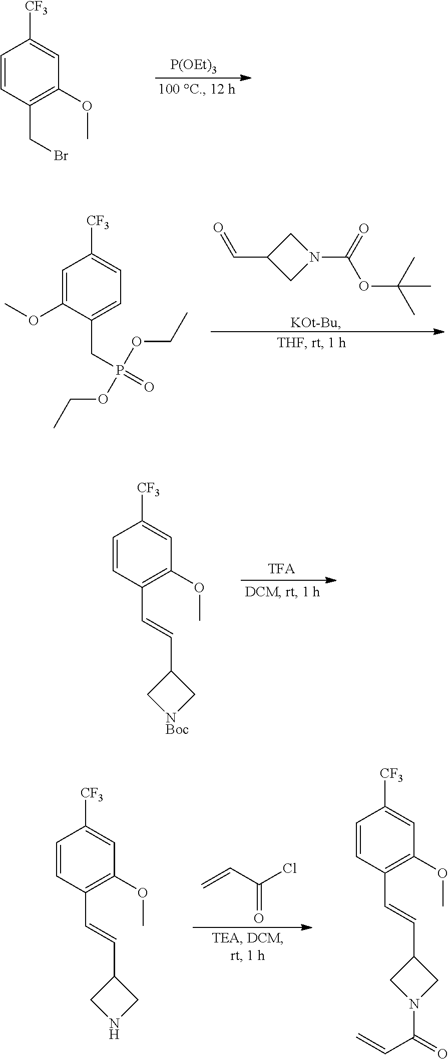

### Synthesis of diethyl (2-methoxy-4-(trifluoromethyl)benzyl)phosphonate

The mixture of 1-(bromomethyl)-2-methoxy-4-(trifluoromethyl)benzene (500 mg, 1.858 mmol) and P(OEt)₃ (5 mL) was heated to 110° C., and stirred overnight, then cooled to r.t, and concentrated under vacuum. The residue was purified by reverse column chromatography with CH₃CN/10 mM NH₄HCO₃ Water (5%-60%) to afford diethyl (2-methoxy-4-(trifluoromethyl)benzyl)phosphonate as a white solid (408 mg, 67.30%). MS (ESI) calcd. for C₁₃H₁₈F₃O₄P, 326.09 m/z, found 327.10 [M+H]⁺.

### Synthesis of tert-butyl 3-(2-methoxy-4-(trifluoromethyl)styryl)azetidine-1-carboxylate

Diethyl (2-methoxy-4-(trifluoromethyl)benzyl)phosphonate (408 mg, 1.251 mmol), a stir bar, tert-butyl 3-formylazetidine-1-carboxylate (278 mg, 1.501 mmol) and THF (7 mL) were added to a 50 mL round-bottom and stirred until homogeneous, and then treated with t-BuOK (211 mg, 1.880 mmol) at 0° C. The resulting mixture was stirred for 2 h at room temperature. The reaction mixture was then quenched with water (20 mL), extracted with EA (3×30 mL), and the combined extracts dried over anhydrous Na₂SO₄, filtered, and concentrated to dryness in vacuo. The residue obtained was then subjected to silica gel chromatography (30%-40% EA/PE) to give tert-butyl 3-(2-methoxy-4-(trifluoromethyl)styryl)azetidine-1-carboxylate as a colorless oil (231 mg). MS (ESI) calcd. for C₁₈H₂₂F₃NO₃, 357.15 m/z, found 302.10 [M+H-56]⁺.

### Synthesis of 3-(2-methoxy-4-(trifluoromethyl)styryl)azetidine 2,2,2-trifluoroacetate

Tert-butyl 3-(2-methoxy-4-(trifluoromethyl)styryl)azetidine-1-carboxylate (231 mg, 0.646 mmol), a stir bar, DCM (5 mL) were added to a 50 mL round-bottom flask and stirred until homogeneous, and then treated with TFA (2 mL). The resulting mixture was stirred for 2 h at room temperature, then concentrated under vacuum to afford 3-(2-methoxy-4-(trifluoromethyl)styryl)azetidine 2,2,2-trifluoroacetate as a red oil (281 mg, crude). MS (ESI) mass calcd. for C₁₃H₁₄F₃NO, 257.10 m/z, found, 258.15 [M+H]⁺.

### Synthesis of 1-(3-(2-methoxy-4-(trifluoromethyl)styryl)azetidin-1-yl)prop-2-en-1-one

3-(2-methoxy-4-(trifluoromethyl)styryl)azetidine 2,2,2-trifluoroacetate (281 mg, 0.757 mmol), a stir bar, TEA (383 mg, 3.785 mmol) and DCM (5 mL) were added to a 50 mL round-bottom flask and stirred until homogeneous, and then treated with acryloyl chloride (137 mg, 1.514 mmol). The resulting mixture was stirred for 1 h at room temperature, then quenched with water (10 mL) and extracted with DCM (10 mL×3). The combined organic extracts were washed with brine, dried over anhydrous Na₂SO₄, filtered and concentrated under vacuum. The residue was purified by HPLC with (Column: XBridge Prep OBD C18 Column, 30*150 mm, 5 μm; Mobile Phase A: Water (10 mmol/L NH₄HCO₃+0.1% NH₃·H₂O), Mobile Phase B: ACN; Flow rate: 60 mL/min; Gradient: 36% B to 66% B in 7 min, 66% B; Wave Length: 254 nm) to afford 1-(3-(2-methoxy-4-(trifluoromethyl)styryl)azetidin-1-yl)prop-2-en-1-one as a white solid (100.2 mg). MS (ESI) mass calcd. for C₁₆H₁₆F₃NO₂, 311.11 m/z, found, 312.15 [M+H]⁺, ¹H NMR (300 MHz, DMSO-d₆) δ 7.78-7.65 (m, 1H), 7.37-7.18 (m, 2H), 6.80-6.59 (m, 2H), 6.39-6.21 (m, 1H), 6.18-6.02 (m, 1H), 5.74-5.60 (m, 1H), 4.54-4.37 (m, 1H), 4.24-4.06 (m, 2H), 3.96-3.75 (m, 4H), 3.62-3.44 (m, 1H). ¹⁹F NMR (282 MHz, DMSO-d₆) δ −60.80.

### Example 20: Synthesis of 1-(6-(4-(trifluoromethyl)styryl)-2-azaspiro[3.3]heptan-2-yl)prop-2-en-1-one

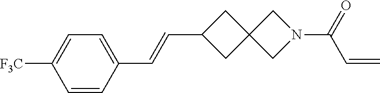 [SMILES: C=CC(=O)N1CC2(CC(/C=C/c3ccc(C(F)(F)F)cc3)C2)C1]

### Synthetic Route:

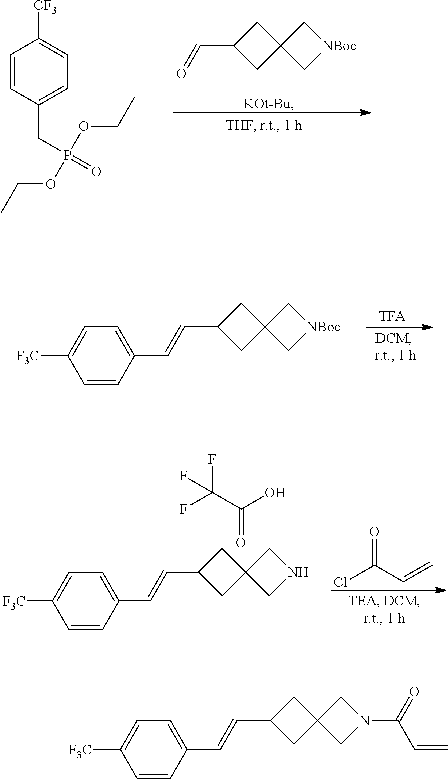

### Synthesis of tert-butyl 6-(4-(trifluoromethyl)styryl)-2-azaspiro[3.3]heptane-2-carboxylate

Diethyl (4-(trifluoromethyl)benzyl)phosphonate (330 mg, 1.114 mmol), a stir bar, tert-butyl 6-formyl-2-azaspiro[3.3]heptane-2-carboxylate (301 mg, 1.336 mmol) and THF (12 mL) were added to a 50 mL round-bottom flask and stirred until homogeneous, and then treated with t-BuOK (188 mg, 1.675 mmol) at 0° C. The resulting mixture was stirred for 2 h at room temperature. The reaction mixture was then quenched with water (20 mL), extracted with EA (3×30 mL), dried over anhdrous Na₂SO₄, filtered, and concentrated to dryness in vacuo. The residue was purified on a silica gel column eluted with EA/PE (10%-30%) to afford tert-butyl 6-(4-(trifluoromethyl)styryl)-2-azaspiro[3.3]heptane-2-carboxylate as a light yellow oil (287 mg). MS (ESI) mass calcd. for C₂₀H₂₄F₃NO₂, 367.18 m/z, found, 312.00 [M+H-56]⁺.

### Synthesis of 6-(4-(trifluoromethyl)styryl)-2-azaspiro[3.3]heptane 2,2,2-trifluoroacetate

Tert-butyl 6-(4-(trifluoromethyl)styryl)-2-azaspiro[3.3]heptane-2-carboxylate (287 mg, 0.781 mmol), a stir bar. DCM (8 mL) were added to a 50 mL round-bottom flask and stirred until homogeneous, and then treated with TFA (3 mL). The reaction mixture was stirred for 2 h at room temperature, then concentrated under vacuum to afford 6-(4-(trifluoromethyl)styryl)-2-azaspiro[3.3]heptane 2,2,2-trifluoroacetate as a red solid (284 mg). MS (ESI) mass calcd. for C₁₅H₁₆F₃N, 267.12 m/z, found, 268.05 [M+H]⁺.

### Synthesis of 1-(6-(4-(trifluoromethyl)styryl)-2-azaspiro[3.3]heptan-2-yl)prop-2-en-1-one

6-(4-(trifluoromethyl)styryl)-2-azaspiro[3.3]heptane 2,2,2-trifluoroacetate (284 mg, 0.745 mmol), a stir bar, Et₃N (377 mg, 3.726 mmol) and DCM (10 mL) were added to a 50 mL round-bottom flask and stirred until homogeneous, and then treated with acryloyl chloride (135 mg, 1.492 mmol). The mixture was stirred for 1 h at room temperature, then quenched with water (20 mL) and extracted with DCM (3×30 mL). The combined organic extracts were washed with brine, dried over anhydrous Na₂SO₄, filtered and concentrated under vacuum. The residue was purified by HPLC with (Column: XBridge Prep OBD C18 Column, 30*150 mm, 5 μm; Mobile Phase A: Water (10 mmol/L NH₄HCO₃+0.1% NH₃·H₂O), Mobile Phase B: ACN; Flow rate: 60 mL/min; Gradient: 45% B to 75% B in 7 min, 75% B; Wave Length: 254 nm) to afford 1-(6-(4-(trifluoromethyl)styryl)-2-azaspiro[3.3]heptan-2-yl)prop-2-en-1-one as a white solid (65.9 mg). MS (ESI) mass calcd. for C₁₈H₁₈F₃NO, 321.13 m/z, found, 322.05 [M+H]⁺, ¹H NMR (300 MHz, DMSO-d₆) δ 7.74-7.57 (m, 4H), 6.64-6.51 (m, 1H), 6.50-6.41 (m, 1H), 6.37-6.20 (m, 1H), 6.13-6.02 (m, 1H), 5.71-5.61 (m, 1H), 4.29 (s, 1H), 4.14 (s, 1H), 4.00 (s, 1H), 3.85 (s, 1H), 3.09-2.91 (m, 1H), 2.48-2.33 (m, 2H), 2.24-2.06 (m, 2H). ¹⁹F NMR (282 MHz, DMSO-d₆) δ −60.80.

### Example 21: Synthesis of (E)-1-(3-(2-(5-(trifluoromethyl)isoxazol-3-yl)vinyl)azetidin-1-yl)prop-2-en-1-one

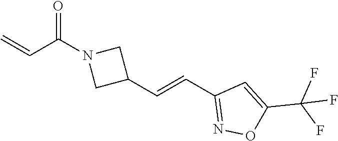 [SMILES: C=CC(=O)N1CC(/C=C/c2cc(C(F)(F)F)on2)C1]

### Synthetic Route:

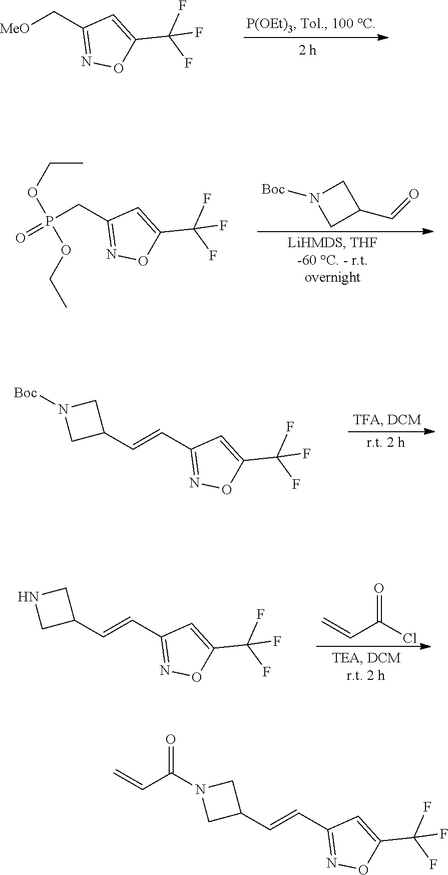

### Synthesis of diethyl [5-(trifluoromethyl)-1,2-oxazol-3-yl]methylphosphonate

To a stirred solution of [5-(trifluoromethyl)-1,2-oxazol-3-yl]methyl methanesulfonate (1 g, 4.079 mmol, 1 equiv) in toluene were added P(OEt)₃ (2.03 g, 12.237 mmol, 3 equiv). The resulting mixture was stirred for 2 h at 100° C. The resulting mixture was concentrated under reduced pressure. The residue was purified by reverse flash chromatography with the following conditions: column, C18 silica gel; mobile phase, MeCN in Water (0.1% TFA), 10% to 50% gradient in 10 min; detector, UV 254 nm. This resulted in diethyl [5-(trifluoromethyl)-1,2-oxazol-3-yl]methylphosphonate (600 mg, 51.22%) as a light yellow oil.

### Synthesis of tert-butyl 3-[(E)-2-[5-(trifluoromethyl)-1,2-oxazol-3-yl]ethenyl]azetidine-1-carboxylate

To a stirred solution of diethyl [5-(trifluoromethyl)-1,2-oxazol-3-yl]methylphosphonate (600 mg, 2.089 mmol, 1 equiv) in THF was added LiHMDS (524.41 mg, 3.133 mmol, 1.5 equiv) dropwise at −60° C. The mixture was stirred for 1 min at −60° C. Next, a solution of tert-butyl 3-formylazetidine-1-carboxylate (464.39 mg, 2.507 mmol, 1.2 equiv) in THF was dropped and stirring was continued at −60° C. for 15 min; then the reaction mixture was allowed to warm up to r.t. overnight. The reaction was quenched by the addition of sat. NH4Cl (aq.) (20 mL) at room temperature. The resulting mixture was extracted with EtOAc (3×30 mL). The combined organic layers were washed with sat. NaCl (aq.) (2×30 mL), dried over anhydrous Na₂SO₄. After filtration, the filtrate was concentrated under reduced pressure. The residue was purified by reverse flash chromatography with the following conditions: column, C18 silica gel; mobile phase, MeCN in Water (0.1% FA), 10% to 50% gradient in 10 min; detector, UV 254 nm. This resulted in tert-butyl 3-[(E)-2-[5-(trifluoromethyl)-1,2-oxazol-3-yl]ethenyl]azetidine-1-carboxylate (200 mg, 30.07%) as a light yellow oil.

### Synthesis of 1-{3-[(E)-2-[5-(trifluoromethyl)-1,2-oxazol-3-yl]ethenyl]azetidin-1-yl}prop-2-en-1-one

To a stirred solution of tert-butyl 3-[(E)-2-[5-(trifluoromethyl)-1,2-oxazol-3-yl]ethenyl]azetidine-1-carboxylate (100 mg, 0.314 mmol, 1 equiv) in DCM was added TFA (0.2 mL) at room temperature. The resulting mixture was stirred for 1 h at room temperature. The crude product was used in the next step directly without further purification.

To a stirred solution of 3-[(E)-2-(azetidin-3-yl)ethenyl]-5-(trifluoromethyl)-1,2-oxazole (100 mg, 0.458 mmol, 1 equiv) in DCM were added TEA (139.14 mg, 1.374 mmol, 3 equiv), the mixture was stirred for 10 min. Next, the acryloyl chloride (62.23 mg, 0.687 mmol, 1.5 equiv) was added to the solution dropwise at 0° C. under nitrogen atmosphere. The resulting mixture was stirred for 2 h at room temperature under nitrogen atmosphere. The resulting mixture was concentrated under reduced pressure. The resulting mixture was extracted with EtOAc (3×10 mL). The combined organic layers were washed with water (2×5 mL), dried over anhydrous Na₂SO₄. After filtration, the filtrate was concentrated under reduced pressure. The crude product (mg) was purified by Prep-HPLC with the following conditions (column, C18 silica gel; mobile phase, MeCN in Water (0.1% FA), 10% to 50% gradient in 10 min; detector, UV 254 nm) to afford 1-{3-[(E)-2-[5-(trifluoromethyl)-1,2-oxazol-3-yl]ethenyl]azetidin-1-yl}prop-2-en-1-one (15.3 mg) as an off-white solid. LC-MS: (ES, m/z); [M+1]=273.0. ¹H NMR (400 MHz, Chloroform-d) δ 7.26 (s, 1H), 6.82 (s, 1H), 6.70-6.48 (m, 2H), 6.36 (dd, J=17.0, 1.8 Hz, 1H), 6.19 (dd, J=17.0, 10.3 Hz, 1H), 5.71 (dd, J=10.3, 1.8 Hz, 1H), 4.43 (s, 2H), 4.06 (s, 2H), 3.56 (m, 1H).

### Example 22: Synthesis of 1-{6-[(E)-2-[5-(trifluoromethyl)-1,2-oxazol-3-yl]ethenyl]-2-azaspiro[3.3]heptan-2-yl}prop-2-en-1-one

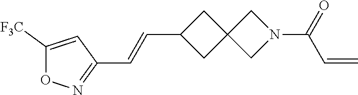 [SMILES: C=CC(=O)N1CC2(CC(/C=C/c3cc(C(F)(F)F)on3)C2)C1]

### Synthetic Route:

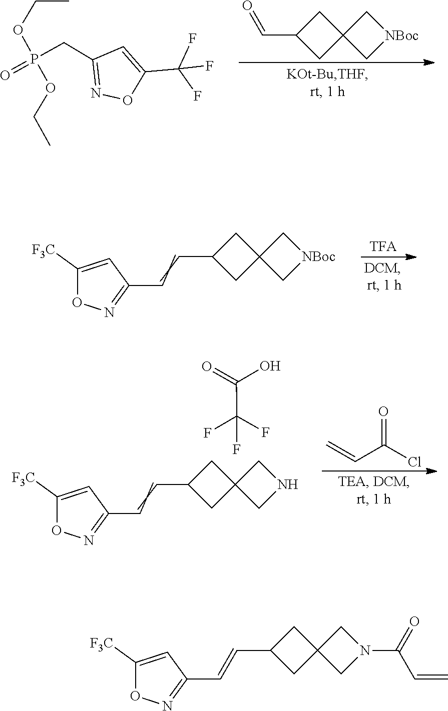

### Synthesis of tert-butyl 6-(2-(5-(trifluoromethyl)isoxazol-3-yl)vinyl)-2-azaspiro[3.3]heptane-2-carboxylate

Diethyl [5-(trifluoromethyl)-1,2-oxazol-3-yl]methylphosphonate (300 mg, 1.045 mmol), tert-butyl 6-formyl-2-azaspiro[3.3]heptane-2-carboxylate (285 mg, 1.265 mmol), a stir bar, and THF (10 mL) were added to a 40 mL vial and stirred until homogeneous, then treated with potassium t-butoxide (176 mg, 1.568 mmol) at 0° C. The resulting mixture stirred for 1 h at r.t., then diluted with water (100 mL) and extracted with EA (100 mL×2). The combined extracts were washed with brine, dried over anhydrous sodium sulfate, filtered, and concentrated to dryness. The residue was subjected to silica gel chromatography (0-30% EtOAc/PE) to afford tert-butyl 6-[-2-[5-(trifluoromethyl)-1,2-oxazol-3-yl]ethenyl]-2-azaspiro[3.3]heptane-2-carboxylate as a colorless oil. MS (ESI) mass calcd. For C₁₇H₂₁F₃N₂O₃, 358.15 m/z, found, 304.15 [M-56+H]⁺

### Synthesis of 3-(2-(2-azaspiro[3.3]heptan-6-yl)vinyl)-5-(trifluoromethyl)isoxazole 2,2,2-trifluoroacetate

Tert-butyl 6-{2-[5-(trifluoromethyl)-1,2-oxazol-3-yl]ethenyl}-2-azaspiro[3.3]heptane-2-carboxylate (370 mg, 1.032 mmol), a stir bar, and DCM (5 mL) were added to a 100 mL round-bottom flask and stirred until homogeneous, and then treated with TFA (1 mL). The resulting mixture was stirred 1 h at r.t., then concentrated under vacuum to afford 3-(2-(2-azaspiro[3.3]heptan-6-yl)vinyl)-5-(trifluoromethyl)isoxazole 2,2,2-trifluoroacetate as a white solid (580 mg). MS (ESI) mass calcd. for C₁₄H₁₄F₆N₂O₃, 258.24 m/z, found, 259.00 [M+H]⁺

### Synthesis of 1-{6-[(E)-2-[5-(trifluoromethyl)-1,2-oxazol-3-yl]ethenyl]-2-azaspiro[3.3]heptan-2-yl}prop-2-en-1-one

3-(2-(2-azaspiro[3.3]heptan-6-yl)vinyl)-5-(trifluoromethyl)isoxazole 2,2,2-trifluoroacetate (580 mg, 2.246 mmol), triethylamine (0.79 g, 7.807 mmol) a stir bar, and DCM (10 mL) were added to a 100 mL round-bottom flask and stirred until homogeneous, then treated with acryloyl chloride (0.41 g, 4.492 mmol) at 0° C. The resulting mixture was stirred at r.t. for 1 h, then diluted with water (100 mL) and extracted with DCM (100 mL×2). The combined extracts were washed with brine, dried over anhydrous sodium sulfate, filtered, and concentrated to dryness to afford crude product, which was purified by PREP-Chiral-HPLC with (Column: CHIRAL ART Cellulose-SC, 2*25 cm, 5 μm; Mobile Phase A: Hex (0.1% 2M NH3-MeOH)—HPLC, Mobile Phase B: EtOH—HPLC; Flow rate: 20 mL/min; Gradient: 30% B to 30% B in 10 min; Wave Length: 220/254 nm; RT2(min): 7.102; Sample Solvent: EtOH—HPLC; Injection Volume: 0.3 mL) to afford 1-{6-[(E)-2-[5-(trifluoromethyl)-1,2-oxazol-3-yl]ethenyl]-2-azaspiro[3.3]heptan-2-yl}prop-2-en-1-one as a white solid. MS (ESI) mass calcd. for C₁₅H₁₅F₃N₂O₂, 312.11 m/z, found, 313.10 [M+H]⁺. ¹H NMR (400 MHz, DMSO-d6) δ 7.83-7.76 (m, 1H), 6.92-6.78 (m, 1H), 6.54-6.43 (m, 1H), 6.34-6.19 (m, 1H), 6.13-5.96 (m, 1H), 5.72-5.56 (m, 1H), 4.33-3.83 (m, 4H), 3.15-2.99 (m, 1H), 2.49-2.40 (m, 2H), 2.23-2.08 (m, 2H). ¹⁹F NMR (376 MHz, DMSO-d₆) δ (ppm): −62.28.

### Example 23: Synthesis of 1-{6-[(Z)-2-[5-(trifluoromethyl)-1,2-oxazol-3-yl]ethenyl]-2-azaspiro[3.3]heptan-2-yl}prop-2-en-1-one

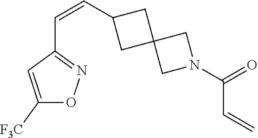 [SMILES: C=CC(=O)N1CC2(CC(/C=C\c3cc(C(F)(F)F)on3)C2)C1]

### Synthetic Route:

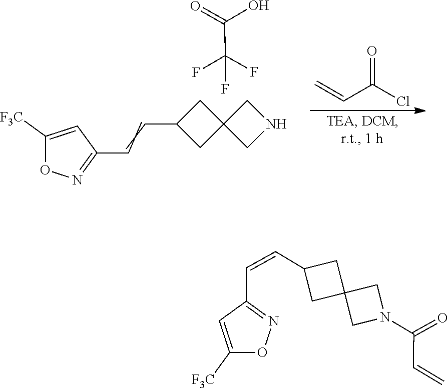

### Synthesis of 1-{6-[(Z)-2-[5-(trifluoromethyl)-1,2-oxazol-3-yl]ethenyl]-2-azaspiro[3.3]heptan-2-yl}prop-2-en-1-one

3-(2-(2-azaspiro[3.3]heptan-6-yl)vinyl)-5-(trifluoromethyl)isoxazole 2,2,2-trifluoroacetate (580 mg, 2.246 mmol), triethylamine (0.79 g, 7.807 mmol) a stir bar, and DCM (10 mL) were added to a 100 mL round-bottom flask and stirred until homogeneous, then treated with acryloyl chloride (0.41 g, 4.492 mmol) at 0° C. The resulting mixture was stirred at r.t. for 1 h, then diluted with water (100 mL) and extracted with DCM (100 mL×2). The combined extracts were washed with brine, dried over anhydrous sodium sulfate, filtered, and concentrated to dryness to afford crude product, which was purified by PREP-Chiral-HPLC with (Column: CHIRAL ART Cellulose-SC, 2*25 cm, 5 μm; Mobile Phase A: Hex (0.1% 2M NH3-MeOH)—HPLC, Mobile Phase B: EtOH—HPLC; Flow rate: 20 mL/min; Gradient: 30% B to 30% B in 10 min; Wave Length: 220/254 nm; RT1(min): 6.862; Sample Solvent: EtOH—HPLC; Injection Volume: 0.3 mL) to afford 1-{6-[(Z)-2-[5-(trifluoromethyl)-1,2-oxazol-3-yl]ethenyl]-2-azaspiro[3.3]heptan-2-yl}prop-2-en-1-one as alight yellow solid (4.8 mg). MS (ESI) mass calcd. for C₁₅H₁₅F₃N₂O₂, 312.11 m/z, found, 313.10 [M+H]⁺. ¹H NMR (400 MHz, DMSO-d6) δ 7.52 (s, 1H), 6.39-6.18 (m, 3H), 6.18-6.01 (m, 1H), 5.74-5.57 (m, 1H), 4.41-3.77 (m, 4H), 3.52 (m, 1H), 2.53-2.34 (m, 2H), 2.23-1.96 (m, 2H). ¹⁹F NMR (376 MHz, DMSO-d6) δ (ppm): −62.94.

### Example 24: Synthesis of 1-{7-[(E)-2-[5-(trifluoromethyl)-1,2-oxazol-3-yl]ethenyl]-2-azaspiro[3.5]nonan-2-yl}prop-2-en-1-one

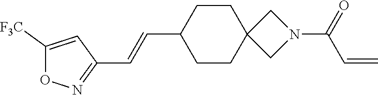 [SMILES: C=CC(=O)N1CC2(CCC(/C=C/c3cc(C(F)(F)F)on3)CC2)C1]

### Synthetic Route:

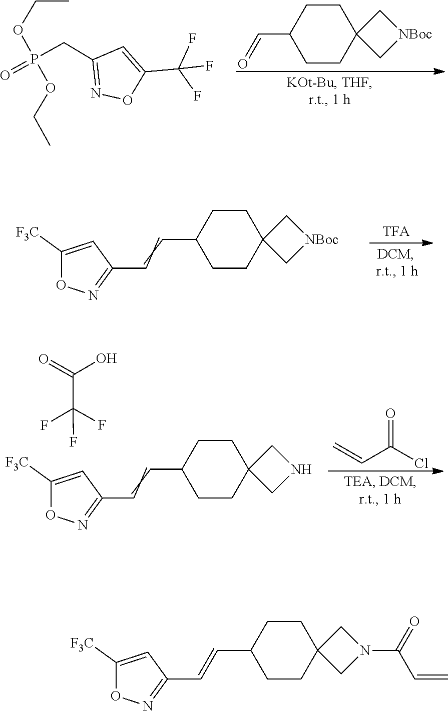

### Synthesis of tert-butyl 7-(2-(5-(trifluoromethyl)isoxazol-3-yl)vinyl)-2-azaspiro[3.5]nonane-2-carboxylate

Diethyl [5-(trifluoromethyl)-1,2-oxazol-3-yl]methylphosphonate (300 mg, 1.045 mmol), tert-butyl 7-formyl-2-azaspiro[3.5]nonane-2-carboxylate (330 mg, 1.303 mmol), a stir bar, and THF (10 mL) were added to a 100 mL round-bottom flask and stirred until homogeneous, then treated with potassium t-butoxide (181 mg, 1.613 mmol) at 0° C. The resulting mixture stirred for 1 h at r.t., then diluted with water (100 mL) and extracted with EA (100 mL×2). The combined extracts were washed with brine, dried over anhydrous sodium sulfate, filtered, and concentrated to dryness. The residue was subjected to silica gel chromatography (0-30% EtOAc/PE) to afford tert-butyl 7-(2-(5-(trifluoromethyl)isoxazol-3-yl)vinyl)-2-azaspiro[3.5]nonane-2-carboxylate as a colorless oil. MS (ESI) mass calcd. for C₁₉H₂₅F₃N₂O₃, 386.18 m/z, found, 331.15 [M-56+H]⁺.

### Synthesis of 3-(2-(2-azaspiro[3.5]nonan-7-yl)vinyl)-5-(trifluoromethyl)isoxazole 2,2,2-trifluoroacetate

Tert-butyl 7-{2-[5-(trifluoromethyl)-1,2-oxazol-3-yl]ethenyl}-2-azaspiro[3.5]nonane-2-carboxylate (360 mg, 0.932 mmol), a stir bar, and DCM (5 mL) were added to a 100 mL round-bottom flask and stirred until homogeneous, and then treated with TFA (1 mL). The resulting mixture was stirred 1 h at r.t., then concentrated under vacuum to afford 3-(2-(2-azaspiro[3.5]nonan-7-yl)vinyl)-5-(trifluoromethyl)isoxazole 2,2,2-trifluoroacetate as a white solid (520 mg). MS (ESI) mass calcd. for C₁₄H₁₄F₆N₂O₃, 258.24 m/z, found, 259.00 [M+H]⁺.

### Synthesis of 1-{7-[(E)-2-[5-(trifluoromethyl)-1,2-oxazol-3-yl]ethenyl]-2-azaspiro[3.5]nonan-2-yl}prop-2-en-1-one

3-(2-(2-azaspiro[3.5]nonan-7-yl)vinyl)-5-(trifluoromethyl)isoxazole 2,2,2-trifluoroacetate (520 mg, 1.353 mmol), triethylamine (685 mg, 6.769 mmol), a stir bar, and DCM (10 mL) were added to a 100 mL round-bottom flask and stirred until homogeneous, then treated with acryloyl chloride (245 mg, 2.707 mmol) at 0° C. The resulting mixture was stirred at r.t. for 1 h, then diluted with water (50 mL) and extracted with DCM (50 mL×2). The combined extracts were washed with brine, dried over anhydrous sodium sulfate, filtered, and concentrated to dryness to afford crude product, which was purified by Prep-HPLC with (Column: XBridge Prep OBD C₁₈ Column, 30*150 mm, 5 μm; Mobile Phase A: Water (10 mmol/L NH₄HCO₃+0.1% NH₃·H₂O), Mobile Phase B: ACN; Flow rate: 60 mL/min; Gradient: 30% B to 60% B in 7 min, 60% B; Wave Length: 254 nm) to afford 1-{7-[(E)-2-[5-(trifluoromethyl)-1,2-oxazol-3-yl]ethenyl]-2-azaspiro[3.5]nonan-2-yl}prop-2-en-1-one as a white solid (76.5 mg). MS (ESI) mass calcd. for C₁₇H₁₉F₃N₂O₂, 340.14 m/z, found, 341.15 [M+H]⁺. ¹H NMR (400 MHz, DMSO-d6) δ 7.59 (s, 1H), 6.71-6.60 (m, 1H), 6.48-6.38 (m, 1H), 6.33-6.19 (m, 1H), 6.14-5.97 (m, 1H), 5.64-5.52 (m, 1H), 3.92-3.52 (m, 4H), 2.21 (s, 1H), 1.96-1.80 (m, 2H), 1.77-1.64 (m, 2H), 1.59-1.38 (m, 2H), 1.31-1.11 (m, 2H). ¹⁹F NMR (376 MHz, DMSO-d₆) δ (ppm): −63.29.

### Example 25: Synthesis of 1-{7-[(Z)-2-[5-(trifluoromethyl)-1,2-oxazol-3-yl]ethenyl]-2-azaspiro[3.5]nonan-2-yl}prop-2-en-1-one

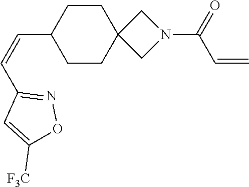 [SMILES: C=CC(=O)N1CC2(CCC(/C=C\c3cc(C(F)(F)F)on3)CC2)C1]

### Synthetic Route:

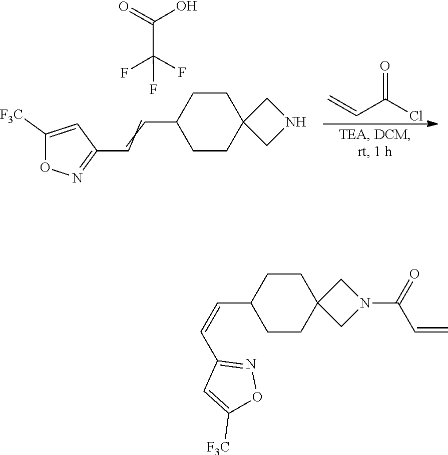

### Synthesis of 1-{7-[(Z)-2-[5-(trifluoromethyl)-1,2-oxazol-3-yl]ethenyl]-2-azaspiro[3.5]nonan-2-yl}prop-2-en-1-one

3-(2-(2-azaspiro[3.5]nonan-7-yl)vinyl)-5-(trifluoromethyl)isoxazole 2,2,2-trifluoroacetate (520 mg, 1.353 mmol), triethylamine (685 mg, 6.769 mmol), a stir bar, and DCM (10 mL) were added to a 100 mL round-bottom flask and stirred until homogeneous, then treated with acryloyl chloride (245 mg, 2.707 mmol) at 0° C. The resulting mixture was stirred at r.t. for 1 h, then diluted with water (50 mL) and extracted with DCM (50 mL×2). The combined extracts were washed with brine, dried over anhydrous sodium sulfate, filtered, and concentrated to dryness to afford crude product, which was purified by Prep-HPLC with (Column: XBridge Prep OBD C₁₈ Column, 30*150 mm, 5 μm; Mobile Phase A: Water (10 mmol/L NH₄HCO₃+0.1% NH₃·H₂O), Mobile Phase B: ACN; Flow rate: 60 mL/min; Gradient: 30% B to 60% B in 7 min, 60% B; Wave Length: 254 nm) to afford 1-{7-[(Z)-2-[5-(trifluoromethyl)-1,2-oxazol-3-yl]ethenyl]-2-azaspiro[3.5]nonan-2-yl}prop-2-en-1-one as a light yellow solid (40.8 mg). MS (ESI) mass calcd. for C₁₇H₁₉F₃N₂O₂, 340.14 m/z, found, 341.15 [M+H]⁺. ¹H NMR (400 MHz, DMSO-d6) δ 7.59 (s, 1H), 6.73-6.60 (m, 2H), 6.47-6.40 (m, 1H), 6.32-6.20 (m, 1H), 6.08-5.98 (m, 1H), 5.63-5.53 (m, 1H), 3.89-3.48 (m, 4H), 2.21 (s, 1H), 1.93-1.82 (m, 2H), 1.77-1.64 (m, 2H), 1.57-1.44 (m, 2H), 1.31-1.12 (m, 2H). ¹⁹F NMR (376 MHz, DMSO-d₆) δ (ppm): −62.93, −62.97, −63.30.

### Example 26: Synthesis of 1-{3-[(Z)-2-[5-(trifluoromethyl)-1,2-oxazol-3-yl]ethenyl]azetidin-1-yl}prop-2-en-1-one

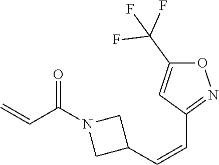 [SMILES: C=CC(=O)N1CC(/C=C\c2cc(C(F)(F)F)on2)C1]

### Synthetic Route:

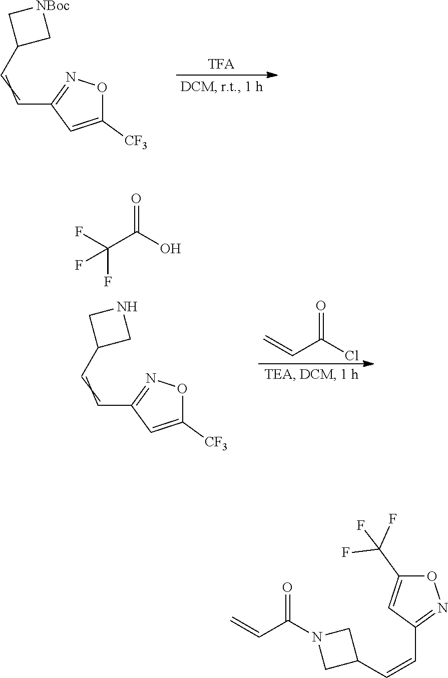

### Synthesis of 3-(2-(azetidin-3-yl)vinyl)-5-(trifluoromethyl)isoxazole 2,2,2-trifluoroacetate

Tert-butyl 3-(2-(5-(trifluoromethyl)isoxazol-3-yl)vinyl)azetidine-1-carboxylate (300 mg, 0.943 mmol), a stir bar, DCM (5 mL) were added to a 100 mL round-bottom flask and stirred until homogeneous, and then treated with TFA (1 mL). The resulting mixture was stirred for 1 h at room temperature, then concentrated under vacuum to afford 3-(2-(azetidin-3-yl)vinyl)-5-(trifluoromethyl)isoxazole 2,2,2-trifluoroacetate as a crude yellow oil (290 mg). MS (ESI), calcd. for C₉H₉F₃N₂O, 218.07 m/z, found 219.10 [M+H]⁺.

### Synthesis of 1-{3-[(Z)-2-[5-(trifluoromethyl)-1,2-oxazol-3-yl]ethenyl]azetidin-1-yl}prop-2-en-1-one

3-(2-(azetidin-3-yl)vinyl)-5-(trifluoromethyl)isoxazole 2,2,2-trifluoroacetate (300 mg, 0.949 mmol), a stir bar, and DCM (10 mL) were added to a 100 mL round-bottom flask and stirred until homogeneous, then treated with triethylamine (480 mg, 4.743 mmol), which was subsequently cooled to 0° C. (ice/water) and charged with acryloyl chloride (172 mg, 1.90 mmol). The resulting mixture was stirred at r.t. for 1 h, then diluted with water (50 mL) and extracted with DCM (50 mL×2). The combined extracts were washed with brine, dried over anhydrous sodium sulfate, filtered, and concentrated to dryness to afford crude product, which was further purified by Prep-HPLC with (Column: CHIRAL ART Cellulose-SC, 2*25 cm, 5 m; Mobile Phase A: Hex (0.5% 2M NH3-MeOH)—HPLC, Mobile Phase B: EtOH—HPLC: Flow rate: 20 mL/min; Gradient: 25% B to 25% B in 13 min; Wave Length: 220/254 nm; RT1(min): 9.204; Sample Solvent: EtOH—HPLC; Injection Volume: 0.5 mL) to afford 1-{3-[(Z)-2-[5-(trifluoromethyl)-1,2-oxazol-3-yl]ethenyl]azetidin-1-yl}prop-2-en-1-one as a yellow solid (6.9 mg). MS (ESI) mass calcd. for C₁₂H₁₁F₃N₂O₂, 272.08 m/z, found, 273.10 [M+H]⁺. ¹H NMR (400 MHz, Chloroform-d) δ 6.64 (s, 1H), 6.47-6.27 (m, 3H), 6.27-6.15 (m, 1H), 5.76-5.67 (m, 1H), 4.68-4.36 (m, 2H), 4.16-3.87 (m, 3H). ¹⁹F NMR (376 MHz, DMSO-d₆) δ (ppm): −64.05.

### Example 27: Synthesis of (S*,E)-1-(3-hydroxy-3-(4-(trifluoromethyl)styryl)pyrrolidin-1-yl)prop-2-en-1-one

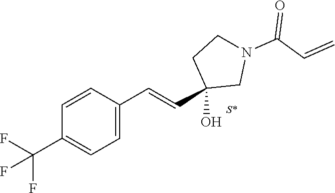 [SMILES: C=CC(=O)N1CC[C@](O)(/C=C/c2ccc(C(F)(F)F)cc2)C1]

### Synthetic Route:

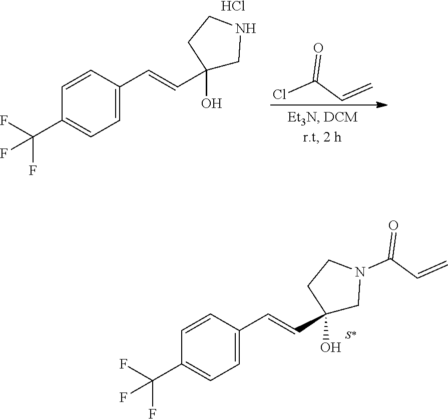

### Synthesis of (S*,E)-1-(3-hydroxy-3-(4-(trifluoromethyl)styryl)pyrrolidin-1-yl)prop-2-en-1-one

3-[(E)-2-[4-(Trifluoromethyl)phenyl]ethenyl]pyrrolidin-3-ol hydrochloride (550 mg, 1.873 mmol), triethylamine (758 mg, 7.492 mmol), a stir bar and DCM (10 mL) were added to a 100 mL round-bottom flask and stirred until homogeneous, then treated with acryloyl chloride (203 mg, 2.248 mmol) in DCM (2 mL) at r.t. The resulting mixture was stirred at r.t. for 2 h. The reaction was quenched with H₂O and extracted with DCM. The organic layers were combined, dried over Na₂SO₄, filtered and concentrated. The residue obtained was purified by Prep-HPLC (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 27% B to 57% B in 7 min, 57% B; Wave Length: 254 nm; RT1(min): 5.75) and Prep-Chiral-HPLC (Column: Lux 5 um Cellulose-2, 2.12*25 cm, 5 μm; Mobile Phase A: Hex (0.5% 2M NH₃-MeOH)—HPLC, Mobile Phase B: EtOH—HPLC; Flow rate: 20 m/mm; Gradient: 10% B to 10% B in 29 min; Wave Length: 220/254 nm; RT2(min): 26.631; Sample Solvent: EtOH—HPLC; Injection Volume: 0.4 mL) to afford (single stereoisomer, absolute stereochemistry randomly assigned as S) (S*,E)-1-(3-hydroxy-3-(4-(trifluoromethyl)styryl)pyrrolidin-1-yl)prop-2-en-1-one (59.8 mg) as a white solid. MS (ESI) mass calcd. for C₁₆H₁₆F₃NO₂, 311.11, found, 312.15 [M+H]⁺; ¹H NMR (400 MHz, DMSO-d6) δ 7.75-7.62 (m, 4H), 6.82 (dd, J=16.0, 4.0 Hz, 1H), 6.74-6.47 (m, 2H), 6.23-6.08 (m, 1H), 5.76-5.59 (m, 1H), 5.45-5.28 (m, 1H), 3.85-3.60 (m, 2H), 3.59-3.37 (m, 2H), 2.21-1.80 (m, 2H); ¹⁹F NMR (376 MHz, DMSO-d₆) δ (ppm): −60.83.

### Example 28: Synthesis of (R*,E)-1-(3-hydroxy-3-(4-(trifluoromethyl)styryl)pyrrolidin-1-yl)prop-2-en-1-one

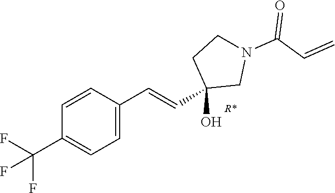 [SMILES: C=CC(=O)N1CC[C@@](O)(/C=C/c2ccc(C(F)(F)F)cc2)C1]

### Synthetic Route:

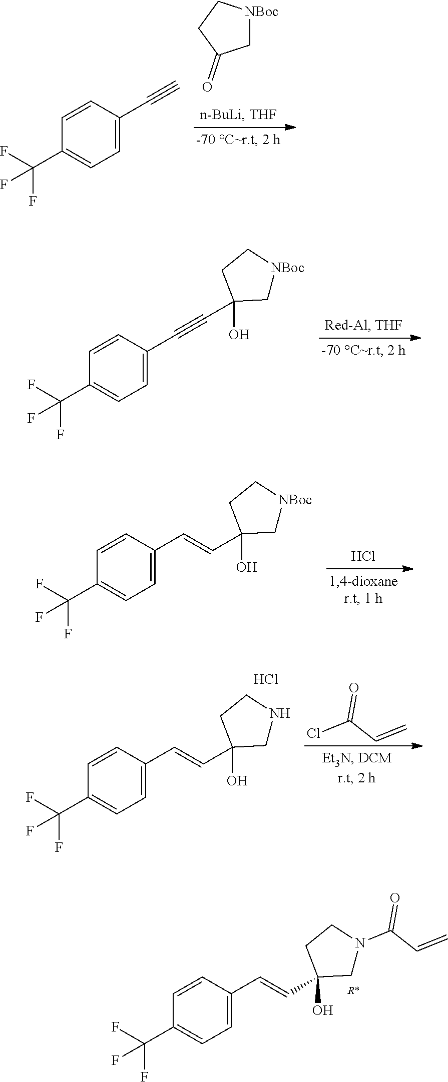

### Synthesis of tert-butyl 3-hydroxy-3-((4-(trifluoromethyl)phenyl)ethynyl)pyrrolidine-1-carboxylate

To a solution of 1-ethynyl-4-(trifluoromethyl)benzene (3.03 g, 17.817 mmol) in THF (35 mL) was added butyllithium (7.77 mL, 19.436 mmol) at −78° C. under nitrogen. After stirring for 30 min, tert-butyl 3-oxopyrrolidine-1-carboxylate (3 g, 16.197 mmol) in THF (15 mL) was added at −78° C. The resulting mixture was stirred at r.t. for 2 h. The reaction was quenched with NH₄Cl and extracted with EA. The organic layers were combined, dried over Na₂SO₄, filtered and concentrated. The residue obtained was purified by silica gel chromatography (EA/PE=0~30%) to afford tert-butyl 3-hydroxy-3-{2-[4-(trifluoromethyl)phenyl]ethynyl}pyrrolidine-1-carboxylate (3.4 g, 59.07%) as an off-white solid. MS (ESI) mass calcd. for C₁₈H₂₀F₃NO₃, 355.15, found, 300.15 [M-56+H]⁺.

### Synthesis of tert-butyl (E)-3-hydroxy-3-(4-(trifluoromethyl)styryl)pyrrolidine-1-carboxylate

To a solution of tert-butyl 3-hydroxy-3-{2-[4-(trifluoromethyl)phenyl]ethynyl}pyrrolidine-1-carboxylate (3.2 g, 9.00 mmol) in THF (60 mL) was added Red-Al (4 mL, 70%) at −78° C. The resulting mixture was warmed to r.t, and stirred for 2 h. EA and sat. aq. potassium sodium tartrate (Rochelle salt) was added, and the mixture was stirred vigorously for 2 h. The organic layers were separated and washed with brine, dried over Na₂SO₄, filtered and concentrated. The residue obtained was purified by silica gel chromatography (EA/PE=0~40%) to afford tert-butyl 3-hydroxy-3-[(E)-2-[4-(trifluoromethyl)phenyl]ethenyl]pyrrolidine-1-carboxylate (2.8 g, 87.01%) as a white solid. MS (ESI) mass calcd. for C₁₈H₂₂F₃NO₃, 357.16, found, 283.95 [M-56-18+H]⁺.

### Synthesis of (E)-3-(4-(trifluoromethyl)styryl)pyrrolidin-3-ol hydrochloride

To a solution of tert-butyl 3-hydroxy-3-[(E)-2-[4-(trifluoromethyl)phenyl]ethenyl]pyrrolidine-1-carboxylate (700 mg, 1.959 mmol) in 1,4-dioxane (15 mL) was added HCl (7 mL, 4 M in dioxane). The resulting mixture was stirred at r.t. for 1.5 h. The resulting mixture was concentrated. The residue obtained was purified by reverse phase chromatography on C18 (ACN/H₂O (0.05% TFA)=5~30%) to afford 3-[(E)-2-[4-(trifluoromethyl)phenyl]ethenyl]pyrrolidin-3-ol hydrochloride (600 mg, crude) as a yellow solid. MS (ESI) mass calcd. for C₁₃H₁₅ClF₃NO: 293.08, found: 257.95 [M−HCl+H]⁺.

### Synthesis of (R*,E)-1-(3-hydroxy-3-(4-(trifluoromethyl)styryl)pyrrolidin-1-yl)prop-2-en-1-one

3-[(E)-2-[4-(Trifluoromethyl)phenyl]ethenyl]pyrrolidin-3-ol hydrochloride (550 mg, 1.873 mmol), triethylamine (758 mg, 7.492 mmol), a stir bar and DCM (10 mL) were added to a 100 mL round-bottom flask and stirred until homogeneous, then treated with acryloyl chloride (203 mg, 2.248 mmol) in DCM (2 mL) at r.t. The resulting mixture was stirred at r.t. for 2 h. The reaction was quenched with H₂O and extracted with DCM. The organic layers were combined, dried over Na₂SO₄, filtered and concentrated. The residue obtained was purified by Prep-HPLC (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 27% B to 57% B in 7 min. 57% B; Wave Length: 254 nm; RT1(min): 5.75) and Prep-Chiral-HPLC (Column: Lux 5 um Cellulose-2, 2.12*25 cm, 5 μm; Mobile Phase A: Hex (0.5% 2M NH₃-MeOH)—HPLC, Mobile Phase B: EtOH—HPLC; Flow rate: 20 mL/min; Gradient: 10% B to 10% B in 29 min; Wave Length: 220/254 nm; RT1(min): 22.356; Sample Solvent: EtOH—HPLC; Injection Volume: 0.4 mL) to afford (single stereoisomer, absolute stereochemistry randomly assigned as R) (R*,E)-1-(3-hydroxy-3-(4-(trifluoromethyl)styryl)pyrrolidin-1-yl)prop-2-en-1-one (62.8 mg) as a white solid. MS (ESI) mass calcd. for C₁₆H₁₆F₃NO₂, 311.11, found, 312.15 [M+H]⁺; ¹H NMR (400 MHz, DMSO-d6) δ 7.76-7.60 (m, 4H), 6.82 (dd, J=16.0, 4.0 Hz, 1H), 6.74-6.47 (m, 2H), 6.22-6.07 (m, 1H), 5.75-5.59 (m, 1H), 5.43-5.29 (m, 1H), 3.85-3.59 (m, 2H), 3.59-3.37 (m, 2H), 2.20-1.81 (m, 2H); ¹⁹F NMR (376 MHz, DMSO-d₆) δ (ppm): −60.83.

### Example 29: Synthesis of 1-[(3S*)-3-methyl-3-{2-[4-(trifluoromethyl)phenyl]ethenyl}pyrrolidin-1-yl]prop-2-en-1-one

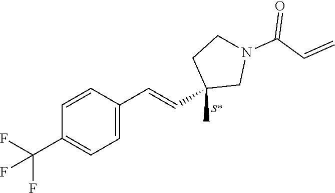 [SMILES: C=CC(=O)N1CC[C@@](C)(/C=C/c2ccc(C(F)(F)F)cc2)C1]

### Synthetic Route:

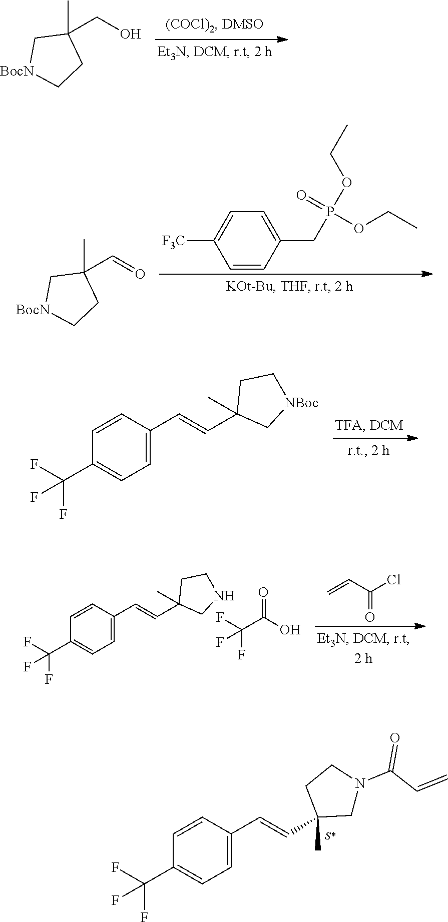

### Synthesis of tert-butyl 3-formyl-3-methylpyrrolidine-1-carboxylate

(COCl)₂ (1.063 g, 8.375 mmol), a stir bar, DCM (10 mL) were added to an oven-dried and nitrogen-purged 100 mL round-bottom flask and stirred until homogenous, then treated with a solution of DMSO (1.306 g, 16.716 mmol) in DCM (3 mL) at −70° C. under nitrogen atmosphere. After stirring for 30 min, tert-butyl 3-(hydroxymethyl)-3-methoxyazetidine-1-carboxylate (900 mg, 4.180 mmol) in DCM (5 mL) was added dropwise and stirring continued for 30 min. To the mixture was added Et₃N (3.382 g, 33.421 mmol) and the resulting mixture was stirred at −70° C. for 30 min, then warmed to room temperature and stirred for 2 h. The reaction mixture was quenched with water and extracted with DCM (300 mL×2). The combined organic layers were washed with water and brine, dried over anhydrous Na₂SO₄, filtered, and concentrated under vacuum to afford tert-butyl 3-formyl-3-methylpyrrolidine-1-carboxylate as a light yellow oil (1 g, crude). MS (ESI) calcd. for C₁₁H₁₉NO₃, 213.10 m/z, found 158.15 [M+H-56]⁺.

### Synthesis of tert-butyl 3-methyl-3-[(E)-2-[4-(trifluoromethyl)phenyl]ethenyl]pyrrolidine-1-carboxylate

Tert-butyl 3-formyl-3-methylpyrrolidine-1-carboxylate (1 g, 4.689 mmol), a stir bar, diethyl [4-(trifluoromethyl)phenyl]methylphosphonate (926 mg, 3.126 mmol), THF (20 mL) were added to a 100 mL round-bottom flask and stirred until homogeneous before the reaction vessel was cooled to 0° C., then treated with potassium 2-methylpropan-2-olate (701 mg, 6.247 mmol). The resulting mixture was stirred for 2 h at r.t., then quenched with water and extracted with EA (100 mL×2). The combined organic layers were washed with water and brine, dried over anhydrous Na₂SO₄, filtered, and concentrated under vacuum. The residue was purified by silica gel chromatography (EA/PE=0~30%) to afford tert-butyl 3-methyl-3-[(E)-2-[4-(trifluoromethyl)phenyl]ethenyl]pyrrolidine-1-carboxylate as a light yellow oil (958 mg, 86.23%). MS (ESI) calcd. for C₁₉H₂₄F₃NO₂, 355.18 m/z, found 300.05 [M+H-56]⁺.

### Synthesis of (E)-3-methyl-3-(4-(trifluoromethyl)styryl)pyrrolidine 2,2,2-trifluoroacetate

Tert-butyl 3-methyl-3-[(E)-2-[4-(trifluoromethyl)phenyl]ethenyl]pyrrolidine-1-carboxylate (958 mg, 2.696 mmol), a stir bar, DCM (10 mL) were added to a 100 mL round-bottom flask and stirred until homogeneous, before the reaction vessel was cooled to 0° C., then treated with TFA (3 mL) dropwise. The resulting mixture was stirred for 2 h at r.t., then concentrated under vacuum to afford (E)-3-methyl-3-(4-(trifluoromethyl)styryl)pyrrolidine 2,2,2-trifluoroacetate as a yellow oil (1.26 g, crude, MS (ESI) calcd. for C₁₄H₁₆F₃N, 255.12 m/z, found 256.05 [M+H]⁺)

### Synthesis of 1-[(3S*)-3-methyl-3-{2-[4-(trifluoromethyl)phenyl]ethenyl}pyrrolidin-1-yl]prop-2-en-1-one

(E)-3-methyl-3-(4-(trifluoromethyl)styryl)pyrrolidine 2,2,2-trifluoroacetate (1.26 g, 3.412 mmol), a stir bar and DCM (10 mL) were added to a 250 mL round-bottom flask, and stirred until homogeneous before the reaction vessel was cooled to 0° C., and charged with Et₃N (1.724 g, 17.037 mmol) over 2 min, and then a solution of acryloyl chloride (615 mg, 6.795 mmol) in DCM (10 mL) was added by dropwise over 5 min. The resulting mixture was stirred for 2 h at r.t., and quenched with water extracted with DCM (100 mL×2). The combined organic layers were dried over anhydrous sodium sulfate, filtered and concentrated under vacuum. The residue was purified by Prep-HPLC (Column: XBridge Prep Phenyl OBD Column, 19*100 mm, 5 μm; Mobile Phase A: Water (10 mmol/L NH₄HCO₃+0.1% NH₃·H₂O), Mobile Phase B: ACN; Flow rate: 60 mL/min; Gradient: 37% B to 67% B in 7 min, 67% B; Wave Length: 254 nm) to afford a racemic product. The racemic product was further separated by SFC (Column: CHIRALPAK IH, 2*25 cm, 5 μm; Mobile Phase A: Hex (0.5% 2M NH₃-MeOH)—HPLC, Mobile Phase B: EtOH—HPLC; Flow rate: 20 mL/min; Gradient: 7% B to 7% B in 18 min; Wave Length: 220/254 nm; RT1(min): 12.588; Sample Solvent: EtOH—HPLC; Injection Volume: 0.3 mL) to afford (single stereoisomer, absolute stereochemistry randomly assigned as S) 1-[(3S*)-3-methyl-3-{2-[4-(trifluoromethyl)phenyl]ethenyl}pyrrolidin-1-yl]prop-2-en-1-one as a colorless oil (119.4 mg). MS (ESI) mass calcd. for C₁₇H₁₈F₃NO, 309.10 m/z, found, 310.15 [M+H]⁺; ¹H NMR (400 MHz, DMSO-d₆) δ 7.66 (m, 4H), 6.65-6.53 (m, 3H), 6.14 (dt, J=16.8, 2.8 Hz, 1H), 5.67 (ddd, J=10.3, 4.3, 2.4 Hz, 1H), 3.71-3.43 (m, 4H), 2.13-1.73 (m, 2H), 1.24 (d, J=3.1 Hz, 3H); ¹⁹F NMR (282 MHz, DMSO-d₆) δ (ppm): −60.82.

### Example 30: Synthesis of 1-[(3R*)-3-methyl-3-{2-[4-(trifluoromethyl)phenyl]ethenyl}pyrrolidin-1-yl]prop-2-en-1-one)

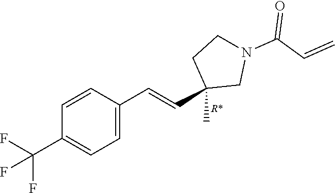 [SMILES: C=CC(=O)N1CC[C@](C)(/C=C/c2ccc(C(F)(F)F)cc2)C1]

### Synthetic Route:

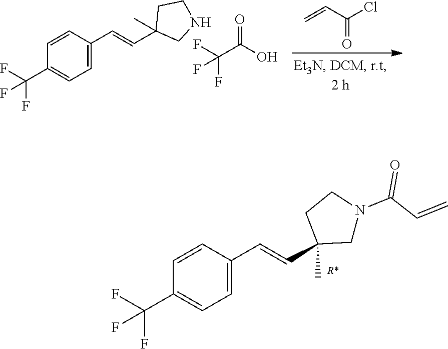

### Synthesis of 1-[(3R*)-3-methyl-3-{2-[4-(trifluoromethyl)phenyl]ethenyl}pyrrolidin-1-yl]prop-2-en-1-one)

(E)-3-methyl-3-(4-(trifluoromethyl)styryl)pyrrolidine 2,2,2-trifluoroacetate (1.26 g, 3.412 mmol), a stir bar and DCM (10 mL) were added to a 250 mL round-bottom flask, and stirred until homogeneous before the reaction vessel was cooled to 0° C., and charged with Et₃N (1.724 g, 17.037 mmol) over 2 min, and then a solution of acryloyl chloride (615 mg, 6.795 mmol) in DCM (10 mL) was added by dropwise over 5 min. The resulting mixture was stirred for 2 h at r.t., and quenched with water extracted with DCM (100 mL×2). The combined organic layers were dried over anhydrous sodium sulfate, filtered and concentrated under vacuum. The residue was purified by Prep-HPLC (Column: XBridge Prep Phenyl OBD Column, 19*100 mm, 5 μm; Mobile Phase A: Water (10 mmol/L NH₄HCO₃+0.1% NH₃·H₂O), Mobile Phase B: ACN; Flow rate: 60 mL/min; Gradient: 37% B to 67% B in 7 min, 67% B; Wave Length: 254 nm; RT1(min): 6) to afford a racemic product. The racemic product was further separated by SFC (Column: CHIRALPAK IH, 2*25 cm, 5 μm; Mobile Phase A: Hex (0.5% 2M NH₃-MeOH)—HPLC, Mobile Phase B: EtOH—HPLC; Flow rate: 20 mL/min; Gradient: 7% B to 7% B in 18 min; Wave Length: 220/254 nm; RT2(min): 15.997; Sample Solvent: EtOH—HPLC; Injection Volume: 0.3 mL) to afford (single stereoisomer, absolute stereochemistry randomly assigned as R) 1-[(3R*)-3-methyl-3-{2-[4-(trifluoromethyl)phenyl]ethenyl}pyrrolidin-1-yl]prop-2-en-1-one) as colorless oil (128.6 mg). MS (ESI) mass calcd. for C₁₇H₁₈F₃NO, 309.10 m/z, found, 310.15 [M+H]⁺; ¹H NMR (400 MHz, DMSO-d₆) δ 7.66 (m, 4H), 6.65-6.52 (m, 3H), 6.14 (ddd, J=16.8, 3.2, 2.4 Hz, 1H), 5.67 (ddd, J=10.3, 4.3, 2.5 Hz, 1H), 3.73-3.41 (m, 4H), 2.13-1.74 (m, 2H), 1.24 (d, J=3.1 Hz, 3H); ¹⁹F NMR (282 MHz, DMSO-d₆) δ (ppm): −60.81.

### Example 31: Synthesis of 1-{3-ethyl-4-[(Z)-2-[4-(trifluoromethyl)phenyl]ethenyl]pyrrolidin-1-yl}prop-2-en-1-one

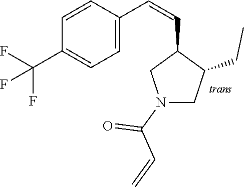 [SMILES: C=CC(=O)N1C[C@@H](CC)[C@H](/C=C\c2ccc(C(F)(F)F)cc2)C1]

### Synthetic Route:

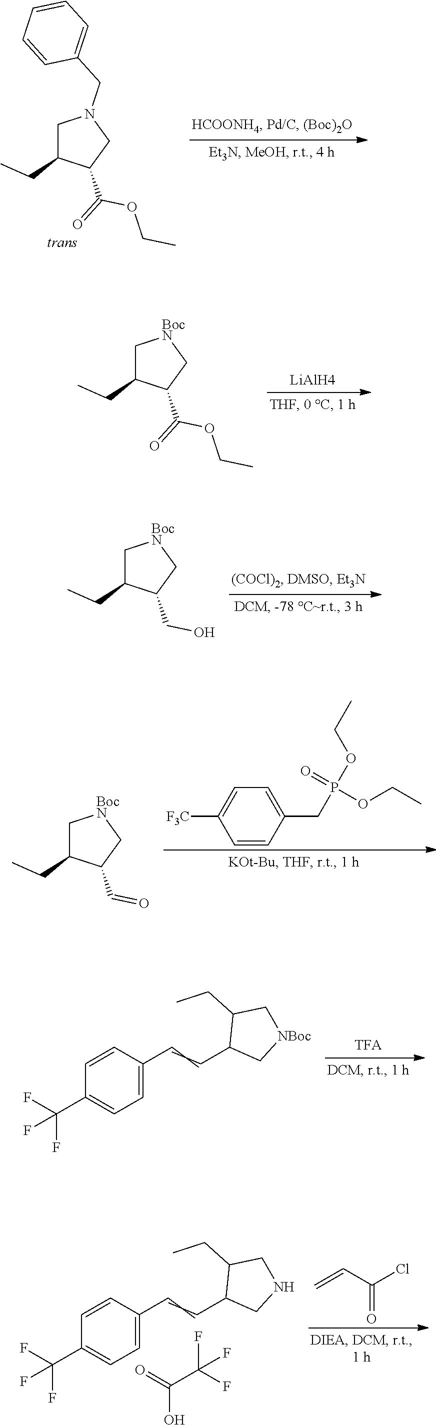

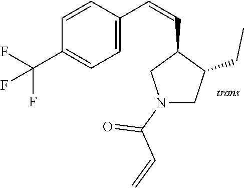 [SMILES: C=CC(=O)N1C[C@@H](CC)[C@H](/C=C\c2ccc(C(F)(F)F)cc2)C1]

### Synthesis of 1-tert-butyl 3-ethyl (3R,4R)-4-ethylpyrrolidine-1,3-dicarboxylate

Ethyl (3R,4R)-1-benzyl-4-ethylpyrrolidine-3-carboxylate (2.5 g, 9.565 mmol), a stir bar, and MeOH (40 mL) were added to a 100 mL round-bottom flask and stirred until homogeneous, and then treated with ammonium formate (1.81 g, 28.705 mmol) and Pd/C (0.25 g). The resulting mixture was stirred for 1 h at room temperature. The resulting mixture was filtered, the filter cake was washed with MeOH (10 mL). Triethylamine (4.84 g, 47.829 mmol) and Ethyl (3R,4R)-1-benzyl-4-ethylpyrrolidine-3-carboxylate (6.26 g, 28.683 mmol) were added to the filtrate. The resulting mixture was stirred for 3 h at room temperature, then concentrated under vacuum. The residue was subjected to silica gel chromatography (0-40% EA/PE) to afford 1-tert-butyl 3-ethyl (3R,4R)-4-ethylpyrrolidine-1,3-dicarboxylate as a colorless oil. MS (ESI) mass calcd. for C₁₄H₂₅NO₄, 271.18 m/z, found 216.15 [M+H-56]⁺.

### Synthesis of tert-butyl (3R,4R)-3-ethyl-4-(hydroxymethyl)pyrrolidine-1-carboxylate

1-tert-butyl 3-ethyl (3R,4R)-4-ethylpyrrolidine-1,3-dicarboxylate (2.5 g, 9.213 mmol), a stir bar, and THF (40 mL) were added to a 250 mL round-bottom flask and stirred until homogeneous, and then treated with 1 M LiAlH₄ (11 mL, 11.000 mmol) at 0° C. The resulting mixture was stirred for 1 h at 0° C., then warmed to r.t., treated with water (0.9 g) and Na₂SO₄. The resulting mixture was filtered, the filter cake was washed with THF. The filtrate was concentrated under reduced pressure. The residue was subjected to silica gel chromatography (0-70% EA/PE) to afford tert-butyl (3R,4R)-3-ethyl-4-(hydroxymethyl)pyrrolidine-1-carboxylate as a colorless oil. MS (ESI) mass calcd. for C₁₂H₂₃NO₃, 229.17 m/z, found 174.05 [M+H-56]⁺.

### Synthesis of tert-butyl (3R,4R)-3-ethyl-4-formylpyrrolidine-1-carboxylate

(COCl)₂ (990 mg, 7.800 mmol) a stir bar, and DCM (15 mL) were added to a 100 mL round-bottom flask and stirred until homogeneous, which was subsequently cooled to −78° C. (dry ice/EtOH) under N₂ and charged with DMSO (1.23 g, 15.743 mmol) dropwise. The resulting mixture was stirred for 0.5 h at −78° C. (dry ice/EtOH) under N₂, then treated with the solution of tert-butyl (3R,4R)-3-ethyl-4-(hydroxymethyl)pyrrolidine-1-carboxylate (900 mg, 3.925 mmol) in DCM (3 mL) dropwise. The resulting mixture was stirred for 0.5 h at −78° C. (dry ice/EtOH) under N₂, then treated with the solution of triethylamine (3.18 g, 31.426 mmol) in DCM (2 mL) dropwise. The resulting mixture was stirred for 10 min at −78° C. (dry ice/EtOH) under N₂, then warmed to room temperature and stirring continued for 2 h. The resulting mixture was quenched with water (150 mL) and extracted with DCM (200 mL×2). The combined extracts were dried over anhydrous sodium sulfate, filtered, and concentrated to dryness to afford tert-butyl (3R,4R)-3-ethyl-4-formylpyrrolidine-1-carboxylate as a yellow oil. MS (ESI), calcd. for C₁₂H₂₁NO₃, 227.15 m/z, found 172.05 [M+H-56]⁺.

### Synthesis of tert-butyl 3-ethyl-4-{2-[4-(trifluoromethyl)phenyl]ethenyl}pyrrolidine-1-carboxylate

Diethyl [4-(trifluoromethyl)phenyl]methylphosphonate (775 mg, 2.616 mmol), tert-butyl (3R,4R)-3-ethyl-4-formylpyrrolidine-1-carboxylate (890 mg, 3.915 mmol), a stir bar, THF (20 mL) were added to a 100 mL round-bottom flask and stirred until homogeneous, then treated with potassium tert-butoxide (587 mg, 5.231 mmol). The resulting mixture was stirred for 1 h at room temperature, then diluted with H₂O (100 mL) and extracted with EtOAc (100 mL×3), the combined organic layers were dried over Na₂SO₄, filtered and concentrated. The residue was subjected to silica gel chromatography (0-25% EA/PE) to afford tert-butyl 3-ethyl-4-{2-[4-(trifluoromethyl)phenyl]ethenyl}pyrrolidine-1-carboxylate as a yellow oil. MS (ESI) mass calcd. for C₂₀H₂₆F₃NO₂, 369.19 m/z, found 314.05 [M+H-56]⁺.

### Synthesis of 3-ethyl-4-(4-(trifluoromethyl)styryl)pyrrolidine 2,2,2-trifluoroacetate

Tert-butyl 3-ethyl-4-{2-[4-(trifluoromethyl)phenyl]ethenyl}pyrrolidine-1-carboxylate (870 mg, 2.355 mmol), a stir bar, and DCM (15 mL) were added to a 100 mL round-bottom flask and stirred until homogeneous, and then treated with TFA (5 mL). The resulting mixture was stirred for 1 h at room temperature, then concentrated under vacuum to afford 3-ethyl-4-(4-(trifluoromethyl)styryl)pyrrolidine 2,2,2-trifluoroacetate (1.34 g, crude) as a yellow solid. MS (ESI) mass calcd. for C₁₇H₁₉F₆NO, 269.14 m/z, found, 270.05 [M+H]⁺.

### Synthesis of 1-{3-ethyl-4-[(Z)-2-[4-(trifluoromethyl)phenyl]ethenyl]pyrrolidin-1-yl}prop-2-en-1-one

3-ethyl-4-(4-(trifluoromethyl)styryl)pyrrolidine 2,2,2-trifluoroacetate (1.34 g, 3.496 mmol), a stir bar, DIEA (2.7 g, 20.890 mmol), DCM (28 mL) were added to a 100 mL round-bottom flask and stirred until homogeneous, then treated with a solution of acryloyl chloride (0.38 g, 4.198 mmol) in DCM (2 mL) dropwise. The mixture was stirred at r.t. for 1 h, then purified by PREP_HPLC (Column: XBridge Prep OBD C18 Column, 30*150 mm, 5 μm; Mobile Phase A: Water (10 mmol/L NH₄HCO₃+0.1% NH₃·H₂O), Mobile Phase B: ACN; Flow rate: 60 mL/min; Gradient: 50% B to 80% B in 7 min, 80% B; Wave Length: 254 nm; RT1(min): 5.25) to afford a crude product. The crude product was further purified by PREP_CHIRAL_HPLC (Column: CHIRAL ART Cellulose-SC, 2*25 cm, 5 μm; Mobile Phase A: MtBE (0.5% 2M NH₃-MeOH)—HPLC, Mobile Phase B: EtOH—HPLC; Flow rate: 20 m/min; Gradient: 7% B to 7% B in 11 min; Wave Length: 220/254 nm; RT1(min): 7.539; Sample Solvent: EtOH—HPLC; Injection Volume: 0.5 mL) to afford 1-{3-ethyl-4-[(Z)-2-[4-(trifluoromethyl)phenyl]ethenyl]pyrrolidin-1-yl}prop-2-en-1-one as an off-white solid. MS (ESI) mass calcd. for C₁₈H₂₀F₃NO, 323.15 m/z, found, 324.10 [M+H]⁺. ¹H NMR (400 MHz, DMSO-d₆) δ 7.62-7.82 (m, 2H), 7.52 (t, J=8.7 Hz, 2H), 6.39-6.88 (m, 2H), 6.13 (dt, J=16.8, 2.6 Hz, 1H), 5.57-5.84 (m, 2H), 3.62-4.13 (m, 2H), 3.40-3.62 (m, 1H), 3.09-3.20 (m, 1H), 2.78-3.05 (m, 1H), 1.79-2.19 (m, 1H), 1.31-1.55 (m, 1H), 1.09-1.21 (m, 1H), 0.65-1.00 (m, 3H). ¹⁹F NMR (376 MHz, DMSO-d₆) δ (ppm): −60.92.

### Example 32: Synthesis of 1-{3-ethyl-4-[(E)-2-[4-(trifluoromethyl)phenyl]ethenyl]pyrrolidin-1-yl}prop-2-en-1-one

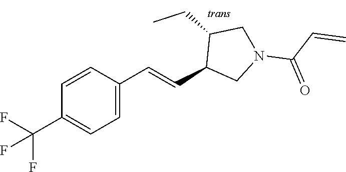 [SMILES: C=CC(=O)N1C[C@@H](CC)[C@H](/C=C/c2ccc(C(F)(F)F)cc2)C1]

Synthetic Route: trans

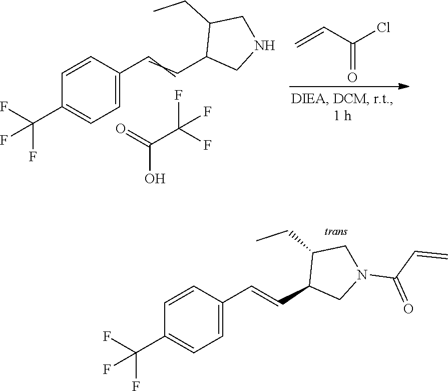

### Synthesis of 1-{3-ethyl-4-[(E)-2-[4-(trifluoromethyl)phenyl]ethenyl]pyrrolidin-1-yl}prop-2-en-1-one

3-ethyl-4-(4-(trifluoromethyl)styryl)pyrrolidine 2,2,2-trifluoroacetate (1.34 g, 3.496 mmol), a stir bar, DIEA (2.7 g, 20.890 mmol), DCM (28 mL) were added to a 100 mL round-bottom flask and stirred until homogeneous, then treated with a solution of acryloyl chloride (0.38 g, 4.198 mmol) in DCM (2 mL) dropwise. The mixture was stirred at r.t. for 1 h, then purified by PREP_HPLC (Column: XBridge Prep OBD C18 Column, 30*150 mm, 5 μm; Mobile Phase A: Water (10 mmol/L NH₄HCO₃+0.1% NH₃·H₂O), Mobile Phase B: ACN; Flow rate: 60 mL/min; Gradient: 50% B to 80% B in 7 min, 80% B; Wave Length: 254 nm; RT1(min): 5.25) to afford a crude product. The crude product was further purified by PREP_CHIRAL_HPLC (Column: CHIRAL ART Cellulose-SC, 2*25 cm, 5 μm; Mobile Phase A: MtBE (0.5% 2M NH₃-MeOH)—HPLC, Mobile Phase B: EtOH—HPLC; Flow rate: 20 m/min; Gradient: 7% B to 7% B in 11 min; Wave Length: 220/254 nm; RT2(min): 8.812; Sample Solvent: EtOH—HPLC; Injection Volume: 0.5 mL) to afford 1-{3-ethyl-4-[(E)-2-[4-(trifluoromethyl)phenyl]ethenyl]pyrrolidin-1-yl}prop-2-en-1-one as a white solid. MS (ESI) mass calcd. for C₁₈H₂₀F₃NO, 323.15 m/z, found, 324.10 [M+H]⁺. ¹H NMR (400 MHz, DMSO-d₆) δ 7.60-7.74 (m, 4H), 6.54-6.72 (m, 2H), 6.30-6.51 (m, 1H), 6.15 (dd, J=16.8, 2.5 Hz, 1H), 5.55-5.80 (m, 1H), 3.70-3.99 (m, 2H), 3.39-3.49, 2.91-3.07 (m, 1H), 3.12-3.27 (m, 1H), 2.55-2.78 (m, 1H), 1.83-2.13 (m, 1H), 1.41-1.71 (m, 1H), 1.10-1.40 (m, 1H), 0.90 (t, J=7.4 Hz, 3H). ¹⁹F NMR (376 MHz, DMSO-d₆) δ (ppm): −60.85.

### Example 33: Synthesis of (S*)-1-(3,3-dimethyl-4-(4-(trifluoromethyl)styryl)pyrrolidin-1-yl)prop-2-en-1-one

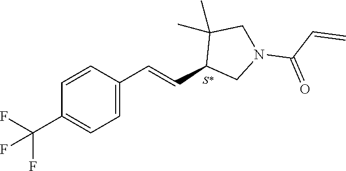 [SMILES: C=CC(=O)N1C[C@@H](/C=C/c2ccc(C(F)(F)F)cc2)C(C)(C)C1]

### Synthetic Route:

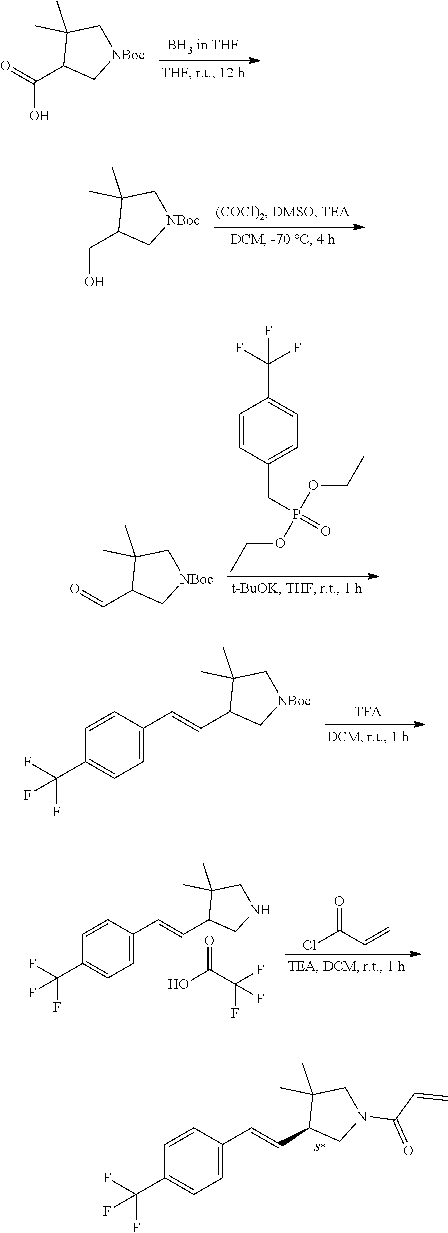

### Synthesis of tert-butyl 4-(hydroxymethyl)-3,3-dimethylpyrrolidine-1-carboxylate

1-(tert-butoxycarbonyl)-4,4-dimethylpyrrolidine-3-carboxylic acid (1 g, 4.110 mmol), a stir bar, and THF (7 mL) were added to a 50 mL round-bottom flask and stirred until homogeneous, and then treated with borane in THF (1 M, 8.2 mL, 8.200 mmol). The resulting mixture was stirred over night at room temperature under a nitrogen atmosphere. The reaction mixture was then quenched with MeOH (15 mL) and stirred for additional 0.5 h, then concentrated under vacuum. The residue was purified by silica gel chromatography (0-5% DCM/MeOH) to afford tert-butyl 4-(hydroxymethyl)-3,3-dimethylpyrrolidine-1-carboxylate as a colorless oil (853 mg, 90.50%). MS (ESI) calcd. for C₁₂H₂₃NO₃, 229.17 m/z, found 174.10 [M+H-56]⁺.

### Synthesis of tert-butyl 4-formyl-3,3-dimethylpyrrolidine-1-carboxylate

To a solution of (COCl)₂ (941 mg, 7.414 mmol) in DCM (4 mL) was added dropwise a solution of DMSO (1158 mg, 14.821 mmol) in DCM (4 mL) at −70° C. under nitrogen atmosphere. After stirring for 30 min, tert-butyl 4-(hydroxymethyl)-3,3-dimethylpyrrolidine-1-carboxylate (850 mg, 3.707 mmol) in DCM (4 mL) was added dropwise and stirring continued for 30 min. To the mixture was added Et₃N (3000 mg, 29.646 mmol) in DCM (4 mL) and the resulting mixture was stirred at −70° C. for 10 min, then warmed to room temperature and stirred for 1 h. The reaction mixture was then quenched with water (20 mL), extracted with DCM (3×40 mL). The combined organic layers were washed with brine, dried over anhydrous Na₂SO₄, filtered, and concentrated under vacuum to afford tert-butyl 4-formyl-3,3-dimethylpyrrolidine-1-carboxylate (825 mg, 97.92%). MS (ESI) calcd. for C₁₂H₂₁NO₃, 227.15 m/z, found 172.05 [M+H-56]⁺.

### Synthesis of tert-butyl 3,3-dimethyl-4-(4-(trifluoromethyl)styryl)pyrrolidine-1-carboxylate

Diethyl (4-(trifluoromethyl)benzyl)phosphonate (800 mg, 2.701 mmol), tert-butyl 4-formyl-3,3-dimethylpyrrolidine-1-carboxylate (737 mg, 3.242 mmol), a stir bar, and THF (16 mg) were added to a 50 mL round-bottom flask and stirred until homogeneous, and then treated with t-BuOK (455 mg, 4.055 mmol) at 0° C. The resulting mixture was stirred for 1 h at room temperature under a nitrogen atmosphere. The reaction mixture was then quenched with water (20 mL), extracted with EA (3×40 mL). The combined organic layers were washed with brine, dried over anhydrous Na₂SO₄, filtered, and concentrated under vacuum. The residue was purified by silica gel chromatography (0-30% EtOAc/PE) to afford tert-butyl 3,3-dimethyl-4-(4-(trifluoromethyl)styryl)pyrrolidine-1-carboxylate as a light yellow oil (842 mg, 84.39%). MS (ESI) calcd. for C₂₀H₂₆F₃NO₂, 369.19 m/z, found 314.10 [M+H-56]¹.

### Synthesis of 3,3-dimethyl-4-(4-(trifluoromethyl)styryl)pyrrolidine 2,2,2-trifluoroacetate

Tert-butyl 3,3-dimethyl-4-(4-(trifluoromethyl)styryl)pyrrolidine-1-carboxylate (840 mg, 2.274 mmol), a stir bar, and DCM (9 mL) were added to a 50 mL round-bottom flask and stirred until homogeneous, and then treated with TFA (3 mL). The resulting mixture was stirred for 1 h at room temperature under a nitrogen atmosphere, then concentrated under vacuum to afford 3,3-dimethyl-4-(4-(trifluoromethyl)styryl)pyrrolidine 2,2,2-trifluoroacetate as a dark yellow oil (1.3 g, crude). MS (ESI) mass calcd. for C₁₅H₁₈F₃N, 296.14 m/z, found, 270.05 [M+H]⁺.

### Synthesis of (S*)-1-(3,3-dimethyl-4-(4-(trifluoromethyl)styryl)pyrrolidin-1-yl)prop-2-en-1-one

3,3-dimethyl-4-(4-(trifluoromethyl)styryl)pyrrolidine 2,2,2-trifluoroacetate (1.3 g, 3.391 mmol), TEA (1.7 g, 16.800 mmol), a stir bar, and DCM (20 mL) were added to a 50 mL round-bottom flask and stirred until homogeneous, and then treated with acryloyl chloride (0.6 g, 6.629 mmol). The resulting mixture was stirred for 1 h at room temperature under a nitrogen atmosphere. The reaction mixture was then quenched with water (20 mL), extracted with DCM (3×40 mL). The combined organic layers were washed with brine, dried over anhydrous Na₂SO₄, filtered, and concentrated under vacuum. The residue was purified by reverse-phase chromatography (5%-50% ACN/10 mM NH₄HCO₃ water) to afford a crude product, which was further purified by Prep-HPLC (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 40% B to 70% B in 7 min, 70% B; Wave Length: 254 nm; RT1(min): 5.87) to afford (single stereoisomer, absolute stereochemistry randomly assigned as S) (S*)-1-(3,3-dimethyl-4-(4-(trifluoromethyl)styryl)pyrrolidin-1-yl)prop-2-en-1-one as colorless oil (83.3 mg, 7.55%). MS (ESI) calcd. for C₁₈H₂₀F₃NO, 323.15 m/z, found 324.10 [M+H]⁺. ¹H NMR (400 MHz, DMSO-d₆) δ 7.76-7.64 (m, 4H), 6.68-6.52 (m, 2H), 6.50-6.38 (m, 1H), 6.15 (ddd, J=16.8, 2.4, 1.3 Hz, 1H), 5.67 (dt. J=10.3, 2.5 Hz, 1H), 3.77 (ddd, J=64.1, 11.4, 8.0 Hz, 1H), 3.65-3.51 (m, 1H), 3.51-3.29 (m, 2H), 2.71 (dd, J=40.9, 8.6 Hz, 1H), 1.08 (s, 3H), 0.91 (s, 3H), ¹⁹F NMR (376 MHz, DMSO-d₆) δ −60.84.

### Example 34: Synthesis of 1-acryloyl-3-(2-(5-(trifluoromethyl)isoxazol-3-yl)vinyl)azetidine-3-carbonitrile

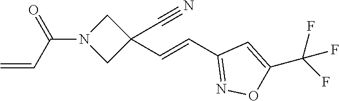 [SMILES: C=CC(=O)N1CC(C#N)(/C=C/c2cc(C(F)(F)F)on2)C1]

### Synthetic Route:

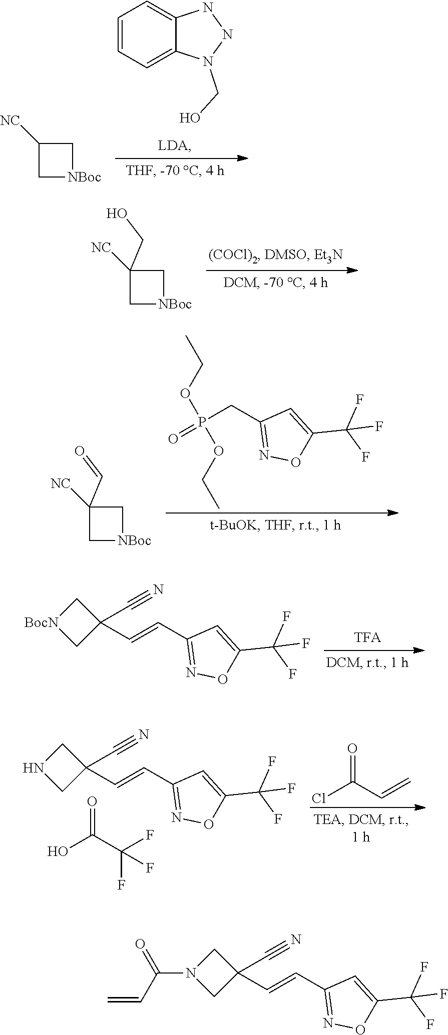

### Synthesis of tert-butyl 3-cyano-3-(hydroxymethyl)azetidine-1-carboxylate

To a solution of diisopropylamine (8.3 g, 82.024 mmol) in THF (20 mL) was added n-Butyllithium in hexanes (2.5 M, 33 mL, 82.500 mmol) at −70° C. under a nitrogen atmosphere. After stirring for 30 min, tert-butyl 3-cyanoazetidine-1-carboxylate (5.0 g, 27.439 mmol) in THF (40 mL) was added and stirring continued for 1 h. To the above mixture was added 1,2,3-benzotriazol-1-ylmethanol (8.2 g, 54.977 mmol). The resulting mixture was stirred for additional 2 h at −70° C. The reaction mixture was then quenched with water (100 mL), extracted with EA (3×200 mL). The combined organic layers were washed with brine, dried over anhydrous Na₂SO₄, filtered, and concentrated under vacuum. The residue was purified by reverse-phase chromatography (5%-50% ACN/0.05% TFA water) to afford tert-butyl 3-cyano-3-(hydroxymethyl)azetidine-1-carboxylate as a white solid (2.0 g, 34.34%). MS (ESI) calcd. for C₁₀H₁₆N₂O₃, 212.12 m/z, found 157.05 [M+H-56]⁺.

### Synthesis of tert-butyl 3-cyano-3-formylazetidine-1-carboxylate

To a solution of (COCl)₂ (1.2 g, 9.455 mmol) in DCM (5 mL) was added dropwise a solution of DMSO (1.5 g, 19.199 mmol) in DCM (5 mL) at −70° C. under nitrogen atmosphere. After stirring for 30 min, tert-butyl 3-cyano-3-(hydroxymethyl)azetidine-1-carboxylate (1 g, 4.711 mmol) in DCM (5 mL) was added dropwise and stirring continued for 30 min. To the mixture was added TEA (3.8 g, 37.552 mmol) in DCM (5 mL) and the resulting mixture was stirred at −70° C. for 10 min, then warmed to room temperature and stirred for 1 h. The reaction mixture was then quenched with water (20 mL), extracted with DCM (3×30 mL). The combined organic layers were washed with brine (30 mL), dried over anhydrous Na₂SO₄, filtered, and concentrated under vacuum to afford tert-butyl 3-cyano-3-formylazetidine-1-carboxylate as a dark yellow viscous oil (1 g, crude). MS (ESI) calcd. for C₁₀H₁₄N2O₃, 210.10 m/z, found 173.00 [M+H-56+18]⁺.

### Synthesis of tert-butyl 3-cyano-3-(2-(5-(trifluoromethyl)isoxazol-3-yl)vinyl)azetidine-1-carboxylate

Diethyl ((5-(trifluoromethyl)isoxazol-3-yl)methyl)phosphonate (300 mg, 1.045 mmol), tert-butyl 3-cyano-3-formylazetidine-1-carboxylate (439 mg, 2.088 mmol), a stir bar, and THF (6 mL) were added to a 50 mL round-bottom flask and stirred until homogeneous, and then treated with t-BuOK (352 mg, 3.137 mmol) at 0° C. The resulting mixture was stirred for 1 h at room temperature under a nitrogen atmosphere. The reaction mixture was then quenched with water (10 mL), extracted with EA (3×20 mL). The combined organic layers were dried over anhydrous Na₂SO₄, filtered, and concentrated under vacuum. The residue was purified by silica gel chromatography (0-30% EtOAc/PE) to afford tert-butyl 3-cyano-3-(2-(5-(trifluoromethyl)isoxazol-3-yl)vinyl)azetidine-1-carboxylate as a yellow solid (147 mg, 40.99%). MS (ESI) calcd. for C₁₅H₁₆F₃N₃O₃, 343.11 m/z, found 288.00 [M+H-56]⁺.

### Synthesis of 3-(2-(5-(trifluoromethyl)isoxazol-3-yl)vinyl)azetidine-3-carbonitrile 2,2,2-trifluoroacetate

Tert-butyl 3-cyano-3-(2-(5-(trifluoromethyl)isoxazol-3-yl)vinyl)azetidine-1-carboxylate (147 mg, 0.428 mmol), a stir bar, DCM (2 mL) were added to a 50 mL round-bottom flask and stirred until homogeneous, and then treated with TFA (0.5 mL). The reaction mixture was stirred for 2 h at room temperature, then concentrated under vacuum to afford 3-(2-(5-(trifluoromethyl)isoxazol-3-yl)vinyl)azetidine-3-carbonitrile 2,2,2-trifluoroacetate as a dark yellow oil (200 mg, crude). MS (ESI) mass calcd. for C₁₀H₈F₃N₃O, 243.06 m/z, found 244.05 [M+H]⁺

### Synthesis of 1-acryloyl-3-(2-(5-(trifluoromethyl)isoxazol-3-yl)vinyl)azetidine-3-carbonitrile

3-(2-(5-(trifluoromethyl)isoxazol-3-yl)vinyl)azetidine-3-carbonitrile 2,2,2-trifluoroacetate (200 mg, 0.560 mmol), TEA (283 mg, 2.797 mmol), a stir bar, and DCM (4 mL) were added to a 50 mL round-bottom flask and stirred until homogeneous, and then treated with acryloyl chloride (101 mg, 1.116 mmol). The resulting mixture was stirred for 1 h at room temperature under a nitrogen atmosphere, then quenched with water (20 mL) and extracted with DCM (3×30 mL). The combined organic extracts were washed with brine, dried over anhydrous Na₂SO₄, filtered and concentrated under vacuum. The residue was purified by HPLC with (Column: Xselect CSH C18 OBD Column 30*150 mm 5 μm, n; Mobile Phase A: Water (0.1% FA), Mobile Phase B: ACN; Flow rate: 60 mL/min; Gradient: 35% B to 65% B in 10 min, 65% B; Wave Length: 254 nm; RT1(min): 8.22) to afford 1-acryloyl-3-(2-(5-(trifluoromethyl)isoxazol-3-yl)vinyl)azetidine-3-carbonitrile as a white solid (25.2 mg). MS (ESI) mass calcd. for C₁₃H₁₀F₃N₃O₂, 297.07 m/z, found, 298.00 [M+H]⁺, ¹H NMR (400 MHz, DMSO-d₆) δ 7.83 (s, 1H), 7.23 (d, J=16.0 Hz, 1H), 6.86 (d, J=16.1 Hz, 1H), 6.31 (dd, J=17.0, 10.2 Hz, 1H), 6.16 (dd, J=17.0, 2.2 Hz, 1H), 5.76 (dd, J=10.3, 2.2 Hz, 1H), 4.84 (d, J=9.1 Hz, 1H), 4.50 (dd, J=17.6, 9.7 Hz, 2H), 4.22 (d, J=10.3 Hz, 1H), ¹⁹F NMR (376 MHz, DMSO-d₆) δ −63.32.

### Example 35: Synthesis of (E)-1-(3-methoxy-3-(4-(trifluoromethyl)styryl)pyrrolidin-1-yl)prop-2-en-1-one

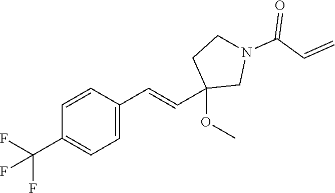 [SMILES: C=CC(=O)N1CCC(/C=C/c2ccc(C(F)(F)F)cc2)(OC)C1]

### Synthetic Route:

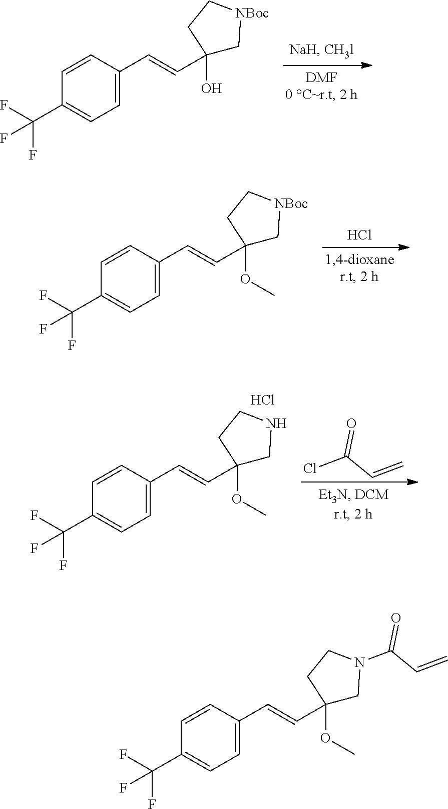

### Synthesis of tert-butyl (E)-3-methoxy-3-(4-(trifluoromethyl)styryl)pyrrolidine-1-carboxylate

To a solution of tert-butyl 3-hydroxy-3-[(E)-2-[4-(trifluoromethyl)phenyl]ethenyl]pyrrolidine-1-carboxylate (700 mg, 1.959 mmol) in DMF (10 mL) was added NaH (94 mg, 2.351 mmol, 60%) under nitrogen at 0° C. After stirring for 30 min, methyl iodide (334 mg, 2.351 mmol) was added. The resulting mixture was stirred at r.t. for 2 h. The reaction was quenched with H₂O and extracted with EA. The organic layers were combined, dried over Na₂SO₄, filtered and concentrated. The residue obtained was purified by silica gel chromatography (EA/PE=0~25%) to afford tert-butyl 3-methoxy-3-[(E)-2-[4-(trifluoromethyl)phenyl]ethenyl]pyrrolidine-1-carboxylate (670 mg, 92.10%) as a colorless oil. MS (ESI) calcd. for C₁₉H₂₄F₃NO₃: 371.17, found: 743.25 [2M+H]⁺.

### Synthesis of (E)-3-methoxy-3-(4-(trifluoromethyl)styryl)pyrrolidine hydrochloride

To a solution of tert-butyl 3-methoxy-3-[(E)-2-[4-(trifluoromethyl)phenyl]ethenyl]pyrrolidine-1-carboxylate (630 mg, 1.696 mmol) in 1,4-dioxane (5 mL) was added HCl (10 mL, 4M in 1,4-dioxane). The resulting mixture was stirred at r.t. for 2 h. The resulting mixture was concentrated to afford 3-methoxy-3-[(E)-2-[4-(trifluoromethyl)phenyl]ethenyl]pyrrolidine hydrochloride (560 mg, crude) as a brown oil. MS (ESI) mass calcd. for C₁₄H₁₇ClF₃NO, 307.10, found, 272.15 [M−HCl+H]⁺.

### Synthesis of (E)-1-(3-methoxy-3-(4-(trifluoromethyl)styryl)pyrrolidin-1-yl)prop-2-en-1-one

To a solution of 3-methoxy-3-[(E)-2-[4-(trifluoromethyl)phenyl]ethenyl]pyrrolidine hydrochloride (560 mg, 1.820 mmol) in DCM (8 mL) was added triethylamine (736 mg, 7.280 mmol) and acryloyl chloride (165 mg, 1.820 mmol) in DCM (2 mL) at r.t. The resulting mixture was stirred at r.t. for 2 h. The reaction was quenched with H₂O and extracted with DCM. The organic layers were combined, dried over Na₂SO₄, filtered and concentrated. The residue obtained was purified by Prep-HPLC (Column: XBridge Prep OBD C18 Column, 30*150 mm, 5Î¼ μm; Mobile Phase A: Water (10 mmol/L NH₄HCO₃), Mobile Phase B: ACN; Flow rate: 60 mL/min; Gradient: 30% B to 60% B in 10 min, 60% B; Wave Length: 254 nm; RT1(min): 8.89) to afford 1-{3-methoxy-3-[(E)-2-[4-(trifluoromethyl)phenyl]ethenyl]pyrrolidin-1-yl}prop-2-en-1-one (22.0 mg) as a yellow semi-solid. MS (ESI) mass calcd. for C₁₇H₁₈F₃NO₂, 325.13, found, 326.05 [M+H]⁺; ¹H NMR (400 MHz, DMSO-d6) δ 7.82-7.64 (m, 4H), 6.83-6.73 (m, 1H), 6.67-6.52 (m, 2H), 6.21-6.09 (m, 1H), 5.75-5.62 (m, 1H), 3.87-3.50 (m, 3H), 3.47-3.37 (m, 1H), 3.16 (d, J=5.7 Hz, 3H), 2.38-1.93 (m, 2H); ¹⁹F NMR (376 MHz, DMSO-d₆) δ (ppm): −60.93.

### Example 36: Synthesis of Example 371-(3-methoxy-4-(4-(trifluoromethyl)styryl)pyrrolidin-1-yl)prop-2-en-1-one

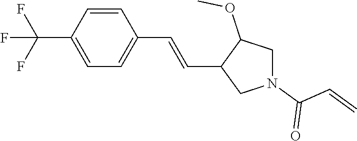 [SMILES: C=CC(=O)N1CC(/C=C/c2ccc(C(F)(F)F)cc2)C(OC)C1]

### Synthetic Route:

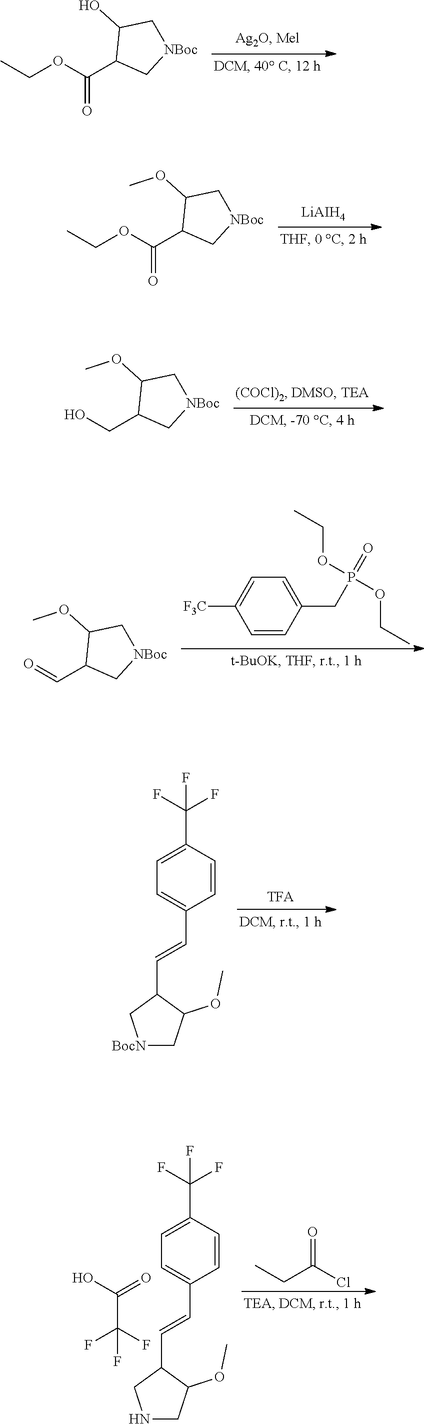

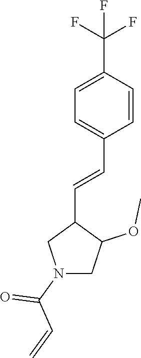 [SMILES: C=CC(=O)N1CC(/C=C/c2ccc(C(F)(F)F)cc2)C(OC)C1]

### Synthesis of 1-tert-butyl 3-ethyl 4-methoxypyrrolidine-1,3-dicarboxylate

1-tert-butyl 3-ethyl 4-hydroxypyrrolidine-1,3-dicarboxylate (1 g, 3.857 mmol), MeI (2.7 g, 19.022 mmol), a stir bar, and DCM (20 mL) were added to a 40 mL vial and stirred until homogeneous, and then treated with Ag₂O (2.7 g, 11.651 mmol). The resulting mixture was stirred over night at 40° C. under a nitrogen atmosphere, then filtered and washed with DCM (mL). The filtrate was washed with brine, dried over anhydrous Na₂SO₄, filtered, and concentrated under vacuum. The residue was purified by silica gel chromatography (20-50% EtOAc/PE) to afford 1-tert-butyl 3-ethyl 4-methoxypyrrolidine-1,3-dicarboxylate as a colorless oil (430 mg, 40.79%). MS (ESI) calcd. for C₁₃H₂₃NO₅, 273.16 m/z, found 218.15 [M+H-56]⁺.

### Synthesis of tert-butyl 3-(hydroxymethyl)-4-methoxypyrrolidine-1-carboxylate

1-tert-butyl 3-ethyl 4-methoxypyrrolidine-1,3-dicarboxylate (430 mg, 1.573 mmol), a stir bar, and THF (4 mL) were added to a 50 mL round-bottom flask and stirred until homogeneous, and then treated with LiAlH₄ in THF (1 M, 1.9 mL, 1.900 mmol) at 0° C. The reaction was allowed to warm to r.t, and stirred for 2 h under a nitrogen atmosphere, then quenched with water (142 mg, 7.865 mmol) and anhydrous Na₂SO₄ and stirred for 20 min. The mixture was filtered, washed with EA (10 mL), and the filtrate was concentrated under vacuum. The residue was purified by silica gel chromatography (50-100% EtOAc/PE) to afford tert-butyl 3-(hydroxymethyl)-4-methoxypyrrolidine-1-carboxylate as a light yellow oil (340 mg, 93.44%). MS (ESI) calcd. for C₁₁H₂₁NO₄, 231.15 m/z, found 176.10 [M+H-56]⁺.

### Synthesis of tert-butyl 3-formyl-4-methoxypyrrolidine-1-carboxylate

To a solution of (COCl)₂ (373 mg, 2.939 mmol) in DCM (2 mL) was added dropwise a solution of DMSO (459 mg, 5.875 mmol) in DCM (1 mL) at −70° C. under nitrogen atmosphere. After stirring for 30 min, tert-butyl 3-(hydroxymethyl)-4-methoxypyrrolidine-1-carboxylate (340 mg, 1.470 mmol) in DCM (2 mL) was added dropwise and stirring continued for 30 min. To the mixture was added TEA (1190 mg, 11.760 mmol) in DCM (1 mL) and the resulting mixture was stirred at −70° C. for 10 min, then warmed to room temperature and stirred for 1 h. The reaction mixture was then quenched with water, extracted with DCM (3×20 mL). The combined organic layers were washed with brine, dried over anhydrous Na₂SO₄, filtered, and concentrated under vacuum to afford tert-butyl 3-formyl-4-methoxypyrrolidine-1-carboxylate as a dark yellow oil (320 mg, 94.95%). MS (ESI) calcd for C₁₁H₁₉NO₄, 229.13 m/z, found 174.20 [M+H-56]⁺.

### Synthesis of tert-butyl 3-methoxy-4-(4-(trifluoromethyl)styryl)pyrrolidine-1-carboxylate

Diethyl (4-(trifluoromethyl)benzyl)phosphonate (320 mg, 1.080 mmol), tert-butyl 3-formyl-4-methoxypyrrolidine-1-carboxylate (297 mg, 1.295 mmol), a stir bar, and THF (6 mg) were added to a 50 mL round-bottom flask and stirred until homogeneous, and then treated with t-BuOK (182 mg, 1.622 mmol) at 0° C. The resulting mixture was stirred for 1 h at room temperature under a nitrogen atmosphere. The reaction mixture was then quenched with water (10 mL), extracted with EA (3×20 mL). The combined organic layers were washed with brine, dried over anhydrous Na₂SO₄, filtered, and concentrated under vacuum. The residue was purified by silica gel chromatography (0-25% EtOAc/PE) to afford tert-butyl 3-methoxy-4-(4-(trifluoromethyl)styryl)pyrrolidine-1-carboxylate as a colorless oil (180 mg, 41.02%). MS (ESI) calcd. for C₁₉H₂₄F₃NO₃, 371.17 m/z, found 316.10 [M+H-56]⁺.

### Synthesis of 3-methoxy-4-(4-(trifluoromethyl)styryl)pyrrolidine 2,2,2-trifluoroacetate

Tert-butyl 3-methoxy-4-(4-(trifluoromethyl)styryl)pyrrolidine-1-carboxylate (180 mg, 0.485 mmol), a stir bar, and DCM (3 mL) were added to a 50 mL round-bottom flask and stirred until homogeneous, and then treated with TFA (1 mL). The resulting mixture was stirred for 1 h at room temperature under a nitrogen atmosphere, then concentrated under vacuum. The residue was purified by reverse-phase chromatography (5%-50% ACN/0.05% TFA water) to afford 3-methoxy-4-(4-(trifluoromethyl)styryl)pyrrolidine 2,2,2-trifluoroacetate as a black solid (174 mg, crude). MS (ESI) mass calcd. for C₁₄H₁₆F₃NO, 271.12 m/z, found, 272.15 [M+H]⁺.

### Synthesis of 1-(3-methoxy-4-(4-(trifluoromethyl)styryl)pyrrolidin-1-yl)prop-2-en-1-one

3-methoxy-4-(4-(trifluoromethyl)styryl)pyrrolidine 2,2,2-trifluoroacetate (147 mg, 0.382 mmol), TEA (193 mg, 1.907 mmol), a stir bar, and DCM (2 mL) were added to a 50 mL round-bottom flask and stirred until homogeneous, and then treated with acryloyl chloride (69 mg, 0.762 mmol). The resulting mixture was stirred for 1 h at room temperature under a nitrogen atmosphere, then quenched with water (20 mL) and extracted with DCM (3×30 mL). The combined organic extracts were washed with brine, dried over anhydrous Na₂SO₄, filtered and concentrated under vacuum. The residue was purified by HPLC with (Column: Xselect CSH C18 OBD Column 30*150 mm 5 m, n; Mobile Phase A: Water (0.1% FA), Mobile Phase B: ACN; Flow rate: 60 mL/min; Gradient: 35% B to 65% B in 7 min, 65% B; Wave Length: 254 nm; RT1(min): 5.25) to afford 1-(3-methoxy-4-(4-(trifluoromethyl)styryl)pyrrolidin-1-yl)prop-2-en-1-one as a light yellow oil (15.1 mg). MS (ESI) mass calcd. for C₁₇H₁₈F₃NO₂, 325.13 m/z, found, 326.05 [M+H]⁺, ¹H NMR (400 MHz, DMSO-d₆) δ 7.76-7.60 (m, 4H), 6.73-6.51 (m, 2H), 6.46 (dd, J=16.0, 7.9 Hz, 1H), 6.15 (dd, J=16.8, 2.4 Hz, 1H), 5.73-5.64 (m, 1H), 3.96-3.88 (m, 1H), 3.88-3.78 (m, 1H), 3.71 (dd, J=12.9, 5.5 Hz, 1H), 3.67-3.56 (m, 1H), 3.56-3.47 (m, 1H), 3.46-3.38 (m, 1H), 3.38-3.26 (m, 2H), 3.20-3.00 (m, 1H). ¹⁹F NMR (376 MHz, DMSO-d₆) δ −60.85.

### Example 37: Synthesis of 1-acryloyl-3-(4-(trifluoromethyl)styryl)azetidine-3-carbonitrile

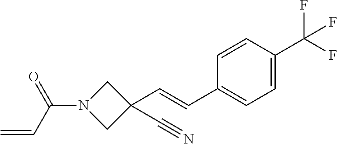 [SMILES: C=CC(=O)N1CC(C#N)(/C=C/c2ccc(C(F)(F)F)cc2)C1]

### Synthetic Route:

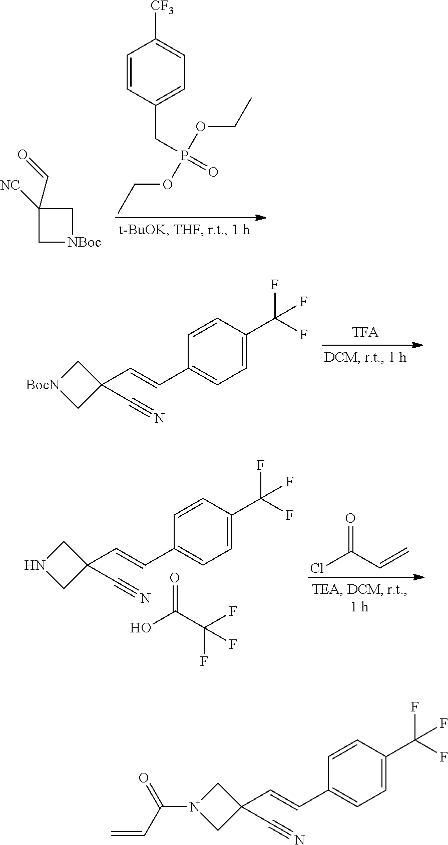

### Synthesis of tert-butyl 3-cyano-3-(4-(trifluoromethyl)styryl)azetidine-1-carboxylate

Diethyl (4-(trifluoromethyl)benzyl)phosphonate (650 mg, 2.194 mmol), tert-butyl 3-cyano-3-formylazetidine-1-carboxylate (554 mg, 2.635 mmol), a stir bar, and THF (10 mL) were added to a 50 mL round-bottom flask and stirred until homogeneous, and then treated with t-BuOK (369 mg, 3.288 mmol) at 0° C. The resulting mixture was stirred for 1 h at room temperature under a nitrogen atmosphere. The reaction mixture was then quenched with water (10 mL), extracted with EA (3×20 mL). The combined organic layers were dried over anhydrous Na₂SO₄, filtered, and concentrated under vacuum. The residue was purified by silica gel chromatography (0-25% EtOAc/PE) to afford tert-butyl 3-cyano-3-(4-(trifluoromethyl)styryl)azetidine-1-carboxylate as a white solid (157 g, 20.31%). MS (ESI) calcd. for C₁₈H₁₉F₃N₂O₂, 352.14 m/z, found 297.05 [M+H]⁺.

### Synthesis of 3-(4-(trifluoromethyl)styryl)azetidine-3-carbonitrile 2,2,2-trifluoroacetate

Tert-butyl 3-cyano-3-(4-(trifluoromethyl)styryl)azetidine-1-carboxylate (150 mg, 0.426 mmol), a stir bar, and DCM (3 mL) were added to a 50 mL round-bottom flask and stirred until homogeneous, and then treated with TFA (1 mL). The resulting mixture was stirred for 1 h at room temperature under a nitrogen atmosphere, then concentrated under vacuum to afford 3-(4-(trifluoromethyl)styryl)azetidine-3-carbonitrile 2,2,2-trifluoroacetate as a light yellow oil (214 mg, crude). MS (ESI) mass calcd. for C₁₃H₁₁F₃N₂, 252.09 m/z, found, 253.00 [M+H]⁺.

### Synthesis of 1-acryloyl-3-(4-(trifluoromethyl)styryl)azetidine-3-carbonitrile

3-(4-(trifluoromethyl)styryl)azetidine-3-carbonitrile (214 mg, 0.604 mmol), TEA (306 mg, 3.024 mmol), a stir bar, and DCM (4 mL) were added to a 50 mL round-bottom flask and stirred until homogeneous, and then treated with acryloyl chloride (109 mg, 1.204 mmol). The resulting mixture was stirred for 1 h at room temperature under a nitrogen atmosphere, then quenched with water (20 mL) and extracted with DCM (3×30 mL). The combined organic extracts were washed with brine, dried over anhydrous Na₂SO₄, filtered and concentrated under vacuum. The residue was purified by HPLC with (Column: Xselect CSH C18 OBD Column 30*150 mm 5 μm, n; Mobile Phase A: Water (0.1% FA), Mobile Phase B: ACN; Flow rate: 60 mL/min; Gradient: 30% B to 60% B in 10 min, 60% B; Wave Length: 254 nm; RT1(min): 8.25) to afford 1-acryloyl-3-(4-(trifluoromethyl)styryl)azetidine-3-carbonitrile as a white solid (39.6 mg). MS (ESI) mass calcd. for C₁₆H₁₃F₃N₂O, 306.10 m/z, found, 307.05 [M+H]⁺, ¹H NMR (400 MHz, DMSO-d₆) δ 7.85-7.68 (m, 4H), 7.02-6.84 (m, 2H), 6.39-6.25 (m, 1H), 6.22-6.12 (m, 1H), 5.81-5.70 (m, 1H), 4.89-4.76 (m, 1H), 4.61-4.51 (m, 1H), 4.49-4.39 (m, 1H), 4.33-4.22 (m, 1H). ¹⁹F NMR (376 MHz, DMSO-d₆) δ −60.99.

### Example 38: Synthesis of 1-(3-((4-(trifluoromethyl)phenyl)ethynyl)pyrrolidin-1-yl)prop-2-en-1-one

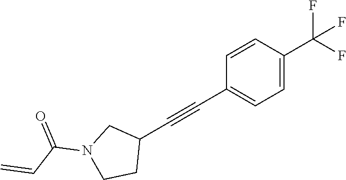 [SMILES: C=CC(=O)N1CCC(C#Cc2ccc(C(F)(F)F)cc2)C1]

### Synthetic Route:

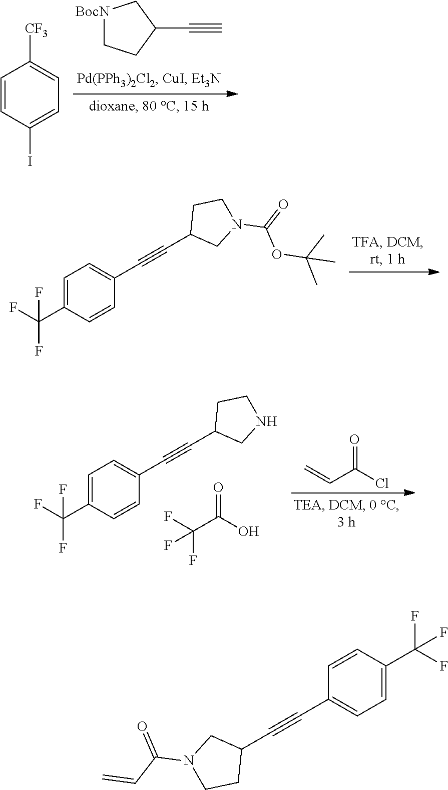

### Synthesis of tert-butyl 3-((4-(trifluoromethyl)phenyl)ethynyl)pyrrolidine-1-carboxylate

1-iodo-4-(trifluoromethyl)benzene (650 mg, 2.390 mmol), a stir bar, TEA (10 mL) and tert-butyl 3-ethynylpyrrolidine-1-carboxylate (466 mg, 2.387 mmol) were added to an oven-dried and nitrogen-purged 25 mL round-bottom flask and stirred until homogenous, then treated with CuI (91 mg, 0.478 mmol) and Pd(PPh₃)₂Cl₂ (335 mg, 0.477 mmol) in batches at rt. The resulting mixture was stirred for 15 h at 80° C. under N2 atmosphere and cooled to room temperature. Then quenched with water and extracted with DCM (10 mL×3). The combined organic layers were washed with brine, dried over anhydrous sodium sulfate, filtered and concentrated under vacuum. The residue was purified by silica gel chromatography (0% to 20% EA in PE) to afford tert-butyl 3-{2-[4-(trifluoromethyl)phenyl]ethynyl}pyrrolidine-1-carboxylate (640 mg, 78.92%) as a yellow solid. MS (ESI) mass calcd. for C₁₈H₂₀F₃NO₂, 339.00 m/z, found 325.15 [M-56+CH₃CN]⁺.

### Synthesis of 3-((4-(trifluoromethyl)phenyl)ethynyl)pyrrolidine 2,2,2-trifluoroacetate

Tert-butyl 3-{2-[4-(trifluoromethyl)phenyl]ethynyl}pyrrolidine-1-carboxylate (640 mg, 1.886 mmol), a stir bar, DCM (6 mL) and TFA (2 mL, 26.926 mmol) were added to an 8 mL vial and stirred until homogenous. The resulting mixture was stirred for 1 h at 25° C., then concentrated under vacuum to afford 3-((4-(trifluoromethyl)phenyl)ethynyl)pyrrolidine 2,2,2-trifluoroacetate (600 mg, crude) as a yellow oil. MS (ESI) mass calcd. for C₁₃H₁₂F₃N, 239.00 m/z, found 240.15 [M+H]⁺.

### Synthesis of 1-(3-((4-(trifluoromethyl)phenyl)ethynyl)pyrrolidin-1-yl)prop-2-en-1-one

3-((4-(trifluoromethyl)phenyl)ethynyl)pyrrolidine 2,2,2-trifluoroacetate (500 mg, 2.090 mmol), a stir bar, DCM (5 mL) and TEA (1056 mg, 10.436 mmol) were added to an 8 mL vial, and stirred until homogenous, then treated with acryloyl chloride (282 mg, 3.116 mmol) dropwise at rt. The resulting mixture was stirred for 3 h at 0° C., then quenched with water. The resulting mixture was extracted with DCM (5 mL×3). The combined organic layers were washed with brine, dried over anhydrous sodium sulfate, filtered and concentrated under vacuum. The residue was purified by Prep-HPLC (Column: XBridge Prep Phenyl OBD Column, 19*250 mm, 5 μm; Mobile Phase A: Water (10 mmol/L NH₄HCO₃), Mobile Phase B: ACN; Flow rate: 25 mL/min; Gradient: 50% B to 65% B in 9 min, 65% B; Wave Length: 254 nm; RT1(min): 8.23) to afford 1-(3-{2-[4-(trifluoromethyl)phenyl]ethynyl}pyrrolidin-1-yl)prop-2-en-1-one (209.1 mg), which was further purified by PREP-CHIRAL-HPLC (Column: CHIRALPAK IA-3, 4.6*50 mm, 3 μm; Mobile Phase A: Hex (0.1% DEA):EtOH=80:20; Flow rate: 1 mL/min; Gradient: 0% B to 0% B; Injection Volume: 5 ul mL) to afford 1-(3-((4-(trifluoromethyl)phenyl)ethynyl)pyrrolidin-1-yl)prop-2-en-1-one (209.1 mg) as a white solid. MS (ESI) calcd. for C₁₆H₁₄F₃NO, 293.00 m/z, found 294.00 [M+H]⁺; ¹H NMR (400 MHz, DMSO-d₆) δ 7.77-7.68 (m, 2H), 7.66-7.59 (m, 2H), 6.66-6.52 (m, 1H), 6.23-6.09 (m, 1H), 5.75-5.63 (m, 1H), 4.00-3.52 (m, 3H), 3.52-3.31 (m, 2H), 2.37-2.18 (m, 1H), 2.14-1.88 (m, 1H); ¹⁹F NMR (376 MHz, DMSO-d₆) δ −61.30.

### Example 39: Synthesis of 1-((3S*,4S*)-3-methyl-4-((E)-4-(trifluoromethyl)styryl)pyrrolidin-1-yl)prop-2-en-1-one

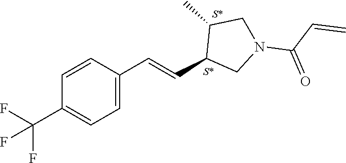 [SMILES: C=CC(=O)N1C[C@@H](C)[C@H](/C=C/c2ccc(C(F)(F)F)cc2)C1]

### Synthetic Route:

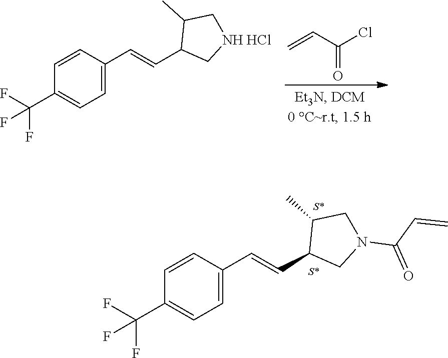

### Synthesis of 1-((3S*,4S*)-3-methyl-4-((E)-4-(trifluoromethyl)styryl)pyrrolidin-1-yl)prop-2-en-1-one

To a solution of 3-methyl-4-[(E)-2-[4-(trifluoromethyl)phenyl]ethenyl]pyrrolidine hydrochloride (400 mg, 1.371 mmol) in DCM (4 mL) was added triethylamine (694 mg, 6.855 mmol) and acryloyl chloride (149 mg, 1.645 mmol) in DCM (1 mL) at 0° C. The resulting mixture was stirred at r.t. for 1.5 h. The reaction was quenched with H₂O and extracted with DCM. The organic layers were combined, dried over Na₂SO₄, filtered and concentrated. The residue obtained was purified by Prep-HPLC (Column: XBridge Prep OBD C18 Column, 30*150 mm, 5 μm; Mobile Phase A: Water (10 mmol/L NH₄HCO₃+0.1% NH₃·H₂O), Mobile Phase B: ACN; Flow rate: 60 mL/min; Gradient: 40% B to 70% B in 7 min, 70% B; Wave Length: 254 nm) and Prep-Chiral-HPLC (Column: CHIRALPAK IE, 2*25 cm, 5 μm; Mobile Phase A: Hex (0.5% 2M NH₃-MeOH)—HPLC, Mobile Phase B: IPA—HPLC; Flow rate: 18 mL/min; Gradient: 30% B to 30% B in 13 min; Wave Length: 220/254 nm; RT2(min): 10.438; Sample Solvent: EtOH—HPLC; Injection Volume: 0.5 mL) to afford (single stereoisomer, absolute stereochemistry randomly assigned as S,S) 1-[(3S*4S*)-3-methyl-4-{2-[4-(trifluoromethyl)phenyl]ethenyl}pyrrolidin-1-yl]prop-2-en-1-one (55.5 mg) as a white solid. MS (ESI) mass calcd. for C₁₇H₁₈F₃NO, 309.13, found, 310.05 [M+H]¹; ¹H NMR (400 MHz, DMSO-d₆) δ 7.74-7.59 (m, 4H), 6.69-6.53 (m, 2H), 6.49-6.36 (m, 1H), 6.15 (dd, J=16.8, 2.5 Hz, 1H), 5.71-5.63 (m, 1H), 3.96-3.86 (m, 1H), 3.85-3.72 (m, 1H), 3.49-3.39 (m, 1H), 3.28-3.10 (m, 1H), 2.98-2.84 (m, 1H), 2.19-1.96 (m, 1H), 1.02 (d, J=6.5 Hz, 3H); ¹⁹F NMR (376 MHz, DMSO-d₆) δ (ppm): −60.84.

### Example 40: Synthesis of 1-[(3R*,4R*)-3-methyl-4-{2-[4-(trifluoromethyl)phenyl]ethenyl}pyrrolidin-1-yl]prop-2-en-1-one

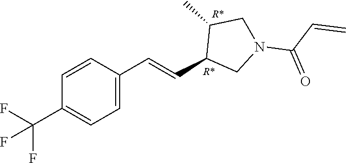 [SMILES: C=CC(=O)N1C[C@@H](C)[C@H](/C=C/c2ccc(C(F)(F)F)cc2)C1]

### Synthetic Route:

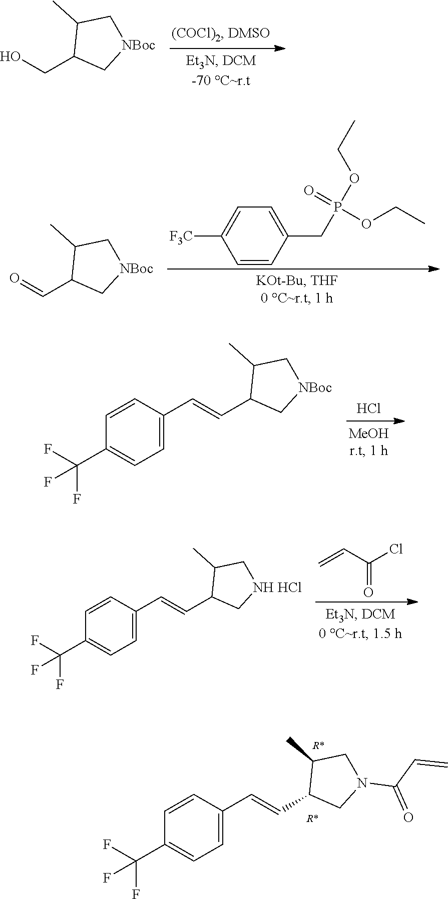

### Synthesis of tert-butyl 3-formyl-4-methylpyrrolidine-1-carboxylate

To a solution of oxalyl chloride (1.05 g, 8.284 mmol) in DCM (10 mL) was added DMSO (1.29 g, 16.568 mmol) in DCM (3 mL) at −70° C. After stirring for 30 min, tert-butoxy[3-(hydroxymethyl)-4-methylpyrrolidin-1-yl]methanol (900 mg, 4.142 mmol) in DCM (4 mL) was added at −70° C. After stirring for 1 h, triethylamine (3.35 g, 33.136 mmol) in DCM (3 mL) was added. The resulting mixture was stirred at −70° C. for 10 min and warmed to r.t. for 2 h. The reaction was quenched with H₂O and extracted with DCM. The organic layers were combined, dried over Na₂SO₄, filtered and concentrated to afford tert-butyl 3-formyl-4-methylpyrrolidine-1-carboxylate (850 mg, crude) as a yellow oil. MS (ESI) mass calcd. for C₁₁H₁₉NO₃, 213.14, found, 158.00 [M-56+H]⁺.

### Synthesis of tert-butyl (E)-3-methyl-4-(4-(trifluoromethyl)styryl)pyrrolidine-1-carboxylate

To a solution of tert-butyl 3-formyl-4-methylpyrrolidine-1-carboxylate (850 mg, 3.985 mmol) and diethyl [4-(trifluoromethyl)phenyl]methylphosphonate (1.42 g, 4.782 mmol) in THF (20 mL) was added t-BuOK (670 mg, 5.978 mmol) at 0° C. After stirring for 10 min, the resulting mixture was warmed to r.t. for 1 h. The reaction was quenched with H₂O and extracted with EA. The organic layers were combined, dried over Na₂SO₄, filtered and concentrated. The residue obtained was purified by silica gel chromatography (EA/PE=0~15%) to afford tert-butyl 3-methyl-4-[(E)-2-[4-(trifluoromethyl)phenyl]ethenyl]pyrrolidine-1-carboxylate (1.04 g, 73.42%) as a yellow oil. MS (ESI) mass calcd. for C₁₉H₂₄F₃NO₂, 355.18, found, 300.05 [M-56+H]⁺.

### Synthesis of (E)-3-methyl-4-(4-(trifluoromethyl)styryl)pyrrolidine hydrochloride

To a solution of tert-butyl 3-methyl-4-[(E)-2-[4-(trifluoromethyl)phenyl]ethenyl]pyrrolidine-1-carboxylate (1 g, 2.814 mmol) in MeOH (5 mL) was added HCl (10 mL, 4 M in 1,4-dioxane). The resulting mixture was stirred at r.t. for 1 h. The resulting mixture was concentrated to afford 3-methyl-4-[(E)-2-[4-(trifluoromethyl)phenyl]ethenyl]pyrrolidine hydrochloride (800 mg, crude). MS (ESI) mass calcd. for C₁₄H₁₇ClF₃N, 291.10, found, 256.10 [M−HCl+H]⁺.

### Synthesis of 1-[(3R*,4R*)-3-methyl-4-{2-[4-(trifluoromethyl)phenyl]ethenyl}pyrrolidin-1-yl]prop-2-en-1-one

To a solution of 3-methyl-4-[(E)-2-[4-(trifluoromethyl)phenyl]ethenyl]pyrrolidine hydrochloride (400 mg, 1.371 mmol) in DCM (4 mL) was added triethylamine (694 mg, 6.855 mmol) and acryloyl chloride (149 mg, 1.645 mmol) in DCM (1 mL) at 0° C. The resulting mixture was stirred at r.t. for 1.5 h. The reaction was quenched with H₂O and extracted with DCM. The organic layers were combined, dried over Na₂SO₄, filtered and concentrated. The residue obtained was purified by Prep-HPLC (Column: XBridge Prep OBD C18 Column, 30*150 mm, 5 μm; Mobile Phase A: Water (10 mmol/L NH₄HCO₃+0.1% NH₃·H₂O), Mobile Phase B: ACN; Flow rate: 60 mL/min; Gradient: 40% B to 70% B in 7 min, 70% B; Wave Length: 254 nm) and Prep-Chiral-HPLC (Column: CHIRALPAK IE, 2*25 cm, 5 μm; Mobile Phase A: Hex (0.5% 2M NH₃-MeOH)—HPLC, Mobile Phase B: IPA—HPLC; Flow rate: 18 mL/min; Gradient: 30% B to 30% B in 13 min; Wave Length: 220/254 nm; RT1(min): 9.179; Sample Solvent: EtOH—HPLC; Injection Volume: 0.5 mL) to afford (single stereoisomer, absolute stereochemistry randomly assigned as R,R) 1-[(3R*,4R*)-3-methyl-4-{2-[4-(trifluoromethyl)phenyl]ethenyl}pyrrolidin-1-yl]prop-2-en-1-one (54.8 mg) as a white solid. MS (ESI) mass calcd. for C₁₇H₁₈F₃NO, 309.13, found, 310.05 [M+H]⁺; ¹H NMR (400 MHz, DMSO-d₆) δ 7.77-7.58 (m, 4H), 6.73-6.53 (m, 2H), 6.50-6.34 (m, 1H), 6.15 (dd, J=16.7, 2.5 Hz, 1H), 5.73-5.59 (m, 1H), 3.98-3.86 (m, 1H), 3.84-3.71 (m, 1H), 3.48-3.40 (m, 1H), 3.28-3.11 (m, 1H), 3.00-2.85 (m, 1H), 2.22-1.94 (m, 1H), 1.02 (d, J=6.5 Hz, 3H); ¹⁹F NMR (376 MHz, DMSO-d₆) δ (ppm): −60.85.

### Example 41: Synthesis of (S*,Z)-1-(3-(3-(trifluoromethyl)styryl)pyrrolidin-1-yl)prop-2-en-1-one

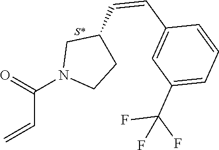 [SMILES: C=CC(=O)N1CC[C@@H](/C=C\c2cccc(C(F)(F)F)c2)C1]

### Synthetic Route:

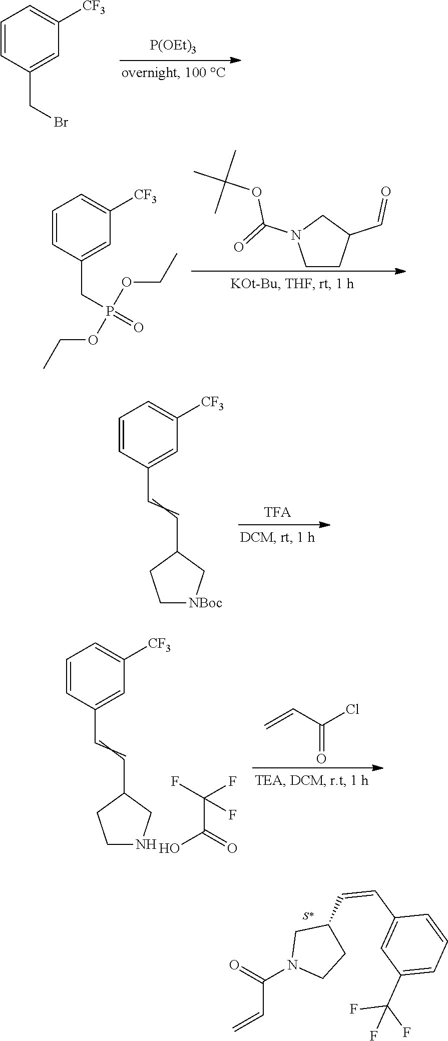

### Synthesis of diethyl [3-(trifluoromethyl)phenyl]methylphosphonate

1-(bromomethyl)-3-(trifluoromethyl)benzene (1 g, 4.183 mmol), a stir bar and triethyl phosphite (10 mL) were added to a 40 mL vial. The reaction mixture was stirred overnight at 100° C., then cooled to r.t, and concentrated under vacuum. The residue was purified by reverse column chromatography (5%-70% CH₃CN/10 mM NH₄HCO₃ Water) to afford diethyl [3-(trifluoromethyl)phenyl]methylphosphonate as a light yellow oil. MS (ESI) mass calcd. for C₁₂H₁₆F₃O₃P, 296.08 m/z, found, 297.10 [M+H]⁺.

### Synthesis of tert-butyl 3-(3-(trifluoromethyl)styryl)pyrrolidine-1-carboxylate

Diethyl [3-(trifluoromethyl)phenyl]methylphosphonate (400 mg, 1.350 mmol), a stir bar, tert-butyl 3-formylazetidine-1-carboxylate (300 mg, 1.506 mmol) and THF (8 mL) were added to a 50 mL round-bottom flask and stirred until homogenous, then treated with t-BuOK (228 mg, 2.032 mmol) at 0° C. The resulting mixture was stirred for 1 h at r.t, then quenched with water (30 mL). The resulting mixture was extracted with EA (50 mL) twice. The combined organic layers were dried over anhydrous sodium sulfate, filtered and concentrated under vacuum. The residue was purified by silica gel chromatography (10-30% EA/PE) to afford tert-butyl 3-(3-(trifluoromethyl)styryl)pyrrolidine-1-carboxylate as yellow oil MS (ESI) mass calcd. for C₁₇H₂₀F₃NO₂, 327.14 m/z, found, 328.14 [M+H]⁺.

### Synthesis of 3-(3-(trifluoromethyl)styryl)pyrrolidine 2,2,2-trifluoroacetate

Tert-butyl 3-(3-(trifluoromethyl)styryl)pyrrolidine-1-carboxylate (500 mg, 1.527 mmol), a stir bar and DCM (10 mL) were added to a 50 mL round-bottom flask and stirred until homogeneous, then treated with TFA (2 mL). The resulting mixture was stirred for 1 h at r.t, then concentrated under vacuum to afford 3-(3-(trifluoromethyl)styryl)pyrrolidine 2,2,2-trifluoroacetate as a yellow oil (600 mg, crude). MS (ESI), calcd. for C₁₃H₁₄F₃N, 241.1 m/z, found 242.05 [M+H]⁺.

### Synthesis of (S*,Z)-1-(3-(3-(trifluoromethyl)styryl)pyrrolidin-1-yl)prop-2-en-1-one

3-(3-(trifluoromethyl)styryl)pyrrolidine 2,2,2-trifluoroacetate (0.63 g, 1.773 mmol), DCM (10 mL), triethylamine (1 g, 9.882 mmol) and a stir bar were added to a 50 mL round-bottom flask and stirred until homogeneous, then treated with acryloyl chloride (0.24 g, 2.652 mmol). The resulting mixture was stirred for 1 h at r.t, then diluted with water (50 mL) and extracted with DCM (50 mL×2). The combined organic layers were washed with brine, dried over anhydrous sodium sulfate, filtered and concentrated to dryness to afford a crude product, which was purified by (Column: XBridge Prep Phenyl OBD Column, 19*250 mm, 5 μm; Mobile Phase A: Water (10 mmol/L NH₄HCO₃+0.1% NH₃·H₂O), Mobile Phase B: ACN; Flow rate: 25 mL/min; Gradient: 40% B to 70% B in 7 min, 70% B; Wave Length: 254 nm; RT1 (min): 6) to afford 1-[3-{2-[3-(trifluoromethyl)phenyl]ethenyl}pyrrolidin-1-yl]prop-2-en-1-one as yellow oil (180 mg). 180 mg of the racemic product was further purified by Chiral HPLC with (Column: CHIRALPAK AS-H, 2*25 cm, 5 μm; Mobile Phase A: Hex (0.5% 2M NH₃-MeOH)—HPLC, Mobile Phase B: EtOH—HPLC; Flow rate: 20 mL/min; Gradient: 10% B to 10% B in 16 min; Wave Length: 220/254 nm; RT1(min): 9.056; and a second purification run (Column: CHIRALPAK IF, 2*25 cm, 5 μm; Mobile Phase A: Hex (0.5% 2M NH3-MeOH)—HPLC, Mobile Phase B: EtOH—HPLC; Flow rate: 20 mL/min; Gradient: 15% B to 15% B in 11.5 min; Wave Length: 220/254 nm; RT1(min): 8.514; Sample Solvent: EtOH—HPLC; Injection Volume: 0.5 mL; Number Of Runs: 6) Sample Solvent: EtOH—HPLC; Injection Volume: 0.4 mL) to afford (as a single stereoisomer, absolute stereochemistry randomly assigned as S) (S*,Z)-1-(3-(3-(trifluoromethyl)styryl)pyrrolidin-1-yl)prop-2-en-1-one as a light yellow oil (3.2 mg). MS (ESI) mass calcd. for C₁₆H₁₆F₃NO, 295.10 m/z, found, 296.10 [M+H]⁺. ¹H NMR (400 MHz, DMSO-d6) δ 7.69-7.55 (m, 4H), 6.65-6.54 (m, 2H), 6.18-6.10 (m, 1H), 5.79-5.62 (m, 2H), 3.90-3.43 (m, 3H), 3.29-3.09 (m, 1H), 2.72-2.51 (m, 1H), 2.34-1.95 (m, 1H), 1.88-1.69 (m, 1H)). ¹⁹F NMR (376 MHz, DMSO-d₆) δ −61.13.

### Example 42: Synthesis of (R*,Z)-1-(3-(3-(trifluoromethyl)styryl)pyrrolidin-1-yl)prop-2-en-1-one

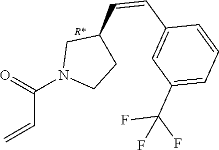 [SMILES: C=CC(=O)N1CC[C@H](/C=C\c2cccc(C(F)(F)F)c2)C1]

### Synthetic Route:

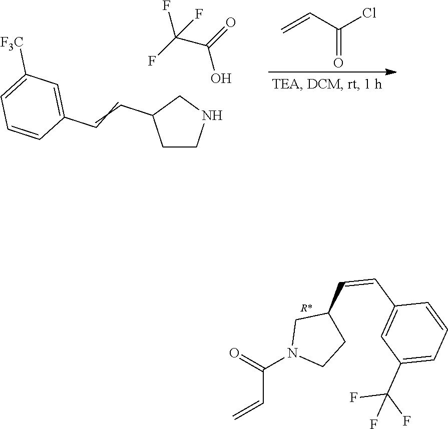

### Synthesis of (R*,Z)-1-(3-(3-(trifluoromethyl)styryl)pyrrolidin-1-yl)prop-2-en-1-one

3-(3-(trifluoromethyl)styryl)pyrrolidine 2,2,2-trifluoroacetate (0.63 g, 1.773 mmol), DCM (10 mL), triethylamine (1 g, 9.882 mmol) and a stir bar were added to a 50 mL round-bottom flask and stirred until homogenous, then treated with acryloyl chloride (0.24 g, 2.652 mmol). The resulting mixture was stirred for 1 h at r.t, then diluted with water (50 mL) and extracted with DCM (50 mL×2). The combined organic layers were washed with brine, dried over anhydrous sodium sulfate, filtered and concentrated to dryness to afford a crude product, which was further purified by (Column: XBridge Prep Phenyl OBD Column, 19*250 mm, 5 μm; Mobile Phase A: Water (10 mmol/L NH₄HCO₃+0.1% NH₃·H₂O), Mobile Phase B: ACN; Flow rate: 25 mL/min; Gradient: 40% B to 70% B in 7 min, 70% B; Wave Length: 254 nm; RT1(min): 6) to afford 1-[3-{2-[3-(trifluoromethyl)phenyl]ethenyl}pyrrolidin-1-yl]prop-2-en-1-one as yellow oil (180 mg). 180 mg of the racemic product was further purified by Chiral HPLC with (Column: CHIRALPAK AS-H, 2*25 cm, 5 μm; Mobile Phase A: Hex (0.5% 2M NH₃-MeOH)—HPLC, Mobile Phase B: EtOH—HPLC; Flow rate: 20 mL/min; Gradient: 10% B to 10% B in 16 min; Wave Length: 220/254 nm; 12.48; Sample Solvent: EtOH—HPLC; Injection Volume: 0.4 mL) to afford (as a single stereoisomer, absolute stereochemistry randomly assigned as R) (R*,Z)-1-(3-(3-(trifluoromethyl)styryl)pyrrolidin-1-yl)prop-2-en-1-one as alight yellow oil (8.2 mg). MS (ESI) mass calcd. for C₁₆H₁₆F₃NO, 295.10 m/z, found, 296.10 [M+H]⁺, ¹H NMR (400 MHz, DMSO-d6) δ 7.70-7.59 (m, 4H), 6.65-6.54 (m, 2H), 6.17-6.11 (m, 1H), 5.8-5.63 (m, 2H), 3.92-3.82 (m, 1H), 3.77-3.67 (m, 1H), 3.64-3.49 (m, 1H), 3.22-3.10 (m, 1H), 2.20-1.97 (m, 1H), 1.92-1.67 (m, 1H), 1.24 (s, 1H). ¹⁹F NMR (376 MHz, DMSO-d₆) δ −61.13.

### Example 43: Synthesis of (R*,E)-1-(3-(3-(trifluoromethyl)styryl)pyrrolidin-1-yl)prop-2-en-1-one

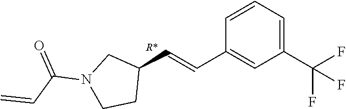 [SMILES: C=CC(=O)N1CC[C@H](/C=C/c2cccc(C(F)(F)F)c2)C1]

### Synthetic Route:

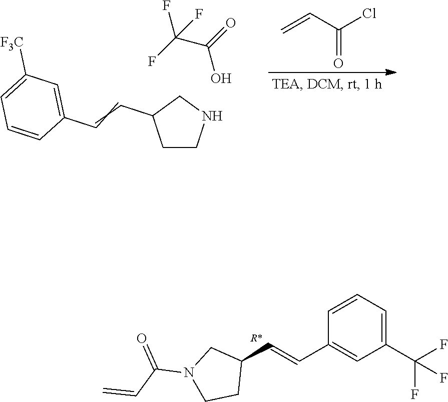

### Synthesis of (R*,E)-1-(3-(3-(trifluoromethyl)styryl)pyrrolidin-1-yl)prop-2-en-1-one

3-(3-(trifluoromethyl)styryl)pyrrolidine 2,2,2-trifluoroacetate (0.63 g, 1.773 mmol), DCM (10 mL), triethylamine (1 g, 9.882 mmol) and a stir bar were added to a 50 mL round-bottom flask and stirred until homogeneous, then treated with acryloyl chloride (0.24 g, 2.652 mmol). The resulting mixture was stirred for 1 h at r.t, then diluted with water (50 mL) and extracted with DCM (50 mL×2). The combined organic layers were washed with brine, dried over anhydrous sodium sulfate, filtered and concentrated to dryness to afford a crude product, which was purified by (Column: XBridge Prep Phenyl OBD Column, 19*250 mm, 5 μm; Mobile Phase A: Water (10 mmol/L NH₄HCO₃+0.1% NH₃·H₂O), Mobile Phase B: ACN; Flow rate: 25 mL/min; Gradient: 40% B to 70% B in 7 min, 70% B; Wave Length: 254 nm; RT1(min): 6) to afford 1-[3-{2-[3-(trifluoromethyl)phenyl]ethenyl}pyrrolidin-1-yl]prop-2-en-1-one as yellow oil (180 mg). 180 mg of the racemic product was further purified by Chiral HPLC with (Column: CHIRALPAK AS-H, 2*25 cm, 5 μm; Mobile Phase A: Hex (0.5% 2M NH₃-MeOH)—HPLC, Mobile Phase B: EtOH—HPLC; Flow rate: 20 mL/min; Gradient: 10% B to 10% B in 16 min; Wave Length: 220/254 nm; RT3(min): 15.095; Sample Solvent: EtOH—HPLC; Injection Volume: 0.4 mL) to afford (single stereoisomer, absolute configuration randomly assigned as R) (R*,E)-1-(3-(3-(trifluoromethyl)styryl)pyrrolidin-1-yl)prop-2-en-1-one as a light yellow oil MS (ESI) mass calcd. for C₁₆H₁₆F₃NO, 295.10 m/z, found, 296.10 [M+H]⁺, ¹H NMR (400 MHz, DMSO-d6) δ 7.81-7.72 (m, 2H), 7.60-7.53 (m, 2H), 6.69-6.48 (m, 3H), 6.19-6.10 (m, 1H), 5.71-5.65 (m, 1H), 3.86-3.81 (m, 1H), 3.79-3.66 (m, 1H), 3.63-3.52 (m, 1H), 3.4-3.15 (m, 1H), 3.13-2.93 (m, 1H), 2.20-2.02 (m, 1H), 1.94-1.74 (m, 1H)). ¹⁹F NMR (376 MHz, DMSO-d₆) δ −61.13.

### Example 44: Synthesis of (S*,E)-1-(3-(3-(trifluoromethyl)styryl)pyrrolidin-1-yl)prop-2-en-1-one

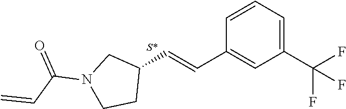 [SMILES: C=CC(=O)N1CC[C@@H](/C=C/c2cccc(C(F)(F)F)c2)C1]

### Synthetic Route:

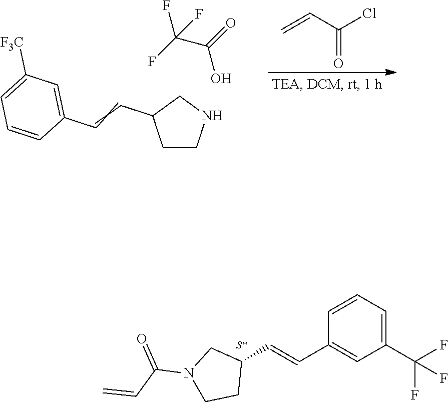

### Synthesis of (S*,E)-1-(3-(3-(trifluoromethyl)styryl)pyrrolidin-1-yl)prop-2-en-1-one

3-(3-(trifluoromethyl)styryl)pyrrolidine 2,2,2-trifluoroacetate (0.63 g, 1.773 mmol), DCM (10 mL), triethylamine (1 g, 9.882 mmol) and a stir bar were added to a 50 mL round-bottom flask and stirred until homogeneous, then treated with acryloyl chloride (0.24 g, 2.652 mmol). The resulting mixture was stirred for 1 h at r.t., then diluted with water (50 mL) and extracted with DCM (50 mL×2). The combined organic layers were washed with brine, dried over anhydrous sodium sulfate, filtered and concentrated to dryness to afford a crude product, which was purified by (Column: XBridge Prep Phenyl OBD Column, 19*250 mm, 5 μm; Mobile Phase A: Water (10 mmol/L NH₄HCO₃+0.1% NH₃·H₂O), Mobile Phase B: ACN; Flow rate: 25 mL/min; Gradient: 40% B to 70% B in 7 min, 70% B; Wave Length: 254 nm; RT1(min): 6) to afford 1-[3-{2-[3-(trifluoromethyl)phenyl]ethenyl}pyrrolidin-1-yl]prop-2-en-1-one as yellow oil (180 mg). 180 mg of the racemic product was further purified by Chiral HPLC with (Column: CHIRALPAK AS-H, 2*25 cm, 5 μm; Mobile Phase A: Hex (0.5% 2M NH₃-MeOH)—HPLC, Mobile Phase B: EtOH—HPLC; Flow rate: 20 mL/min; Gradient: 10% B to 10% B in 16 min; Wave Length: 220/254 nm; RT1(min): 9.056; Sample Solvent: EtOH—HPLC; Injection Volume: 0.4 mL), and then a second purification run (Column: CHIRALPAK IF, 2*25 cm, 5 μm; Mobile Phase A: Hex (0.5% 2M NH3-MeOH)—HPLC. Mobile Phase B: EtOH—HPLC; Flow rate: 20 mL/min; Gradient: 15% B to 15% B in 11.5 min; Wave Length: 220/254 nm; RT2(min): 10.22; Sample Solvent: EtOH—HPLC; Injection Volume: 0.5 m) to afford (single stereoisomer, absolute configuration randomly assigned as S) (S*,E)-1-(3-(3-(trifluoromethyl)styryl)pyrrolidin-1-yl)prop-2-en-1-one as light yellow oil MS (ESI) mass calcd. for C₁₆H₁₆F₃NO, 295.10 m/z, found, 296.10 [M+H]⁺. ¹H NMR (400 MHz, DMSO-d6) δ 7.80-7.72 (m, 2H), 7.60-7.54 (m, 2H), 6.69-6.45 (m, 3H), 6.19-6.11 (m, 1H), 5.69-5.66 (m, 1H), 3.87-3.48 (m, 3H), 3.39-3.17 (m, 1H), 3.11-2.95 (m, 1H), 2.19-2.01 (m, 1H), 1.91-1.71 (m, 1H)). ¹⁹F NMR (376 MHz, DMSO-d₆) δ −61.13.

### Example 45: Synthesis of (E)-1-(3-(3-ethynyl-4-(trifluoromethyl)styryl)azetidin-1-yl)prop-2-en-1-one

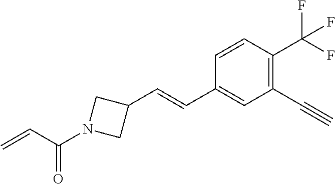 [SMILES: C#Cc1cc(/C=C/C2CN(C(=O)C=C)C2)ccc1C(F)(F)F]

### Synthetic Route:

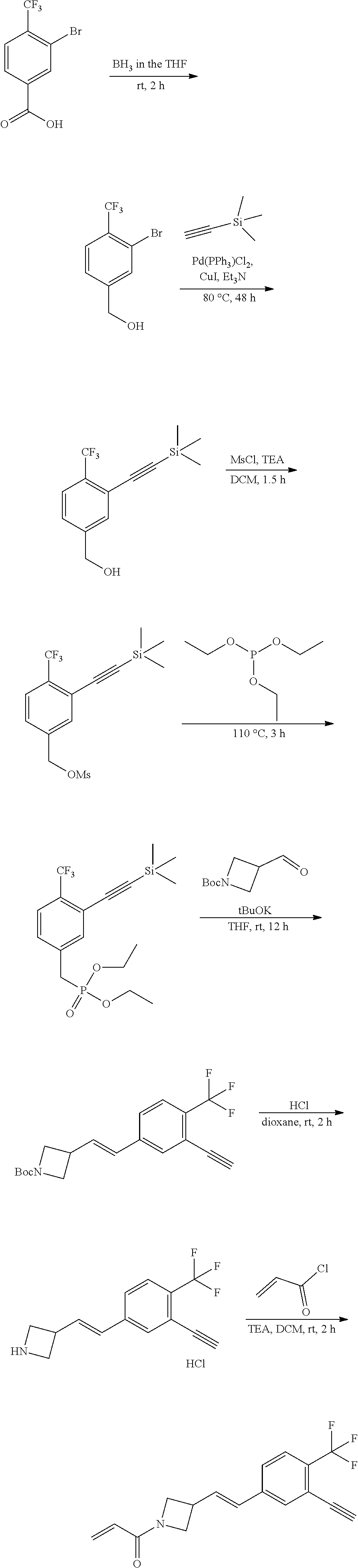

### Synthesis of (3-bromo-4-(trifluoromethyl)phenyl)methanol

3-bromo-4-(trifluoromethyl)benzoic acid (4 g, 14.869 mmol), a stir bar and BH₃ in the THF (40 mL) were added to a 100 mL round-bottom flask and stirred until homogenous at rt. The resulting mixture was stirred for 2 h at 25° C., then quenched with water. The resulting mixture was extracted with EA (40 mL×3). The combined organic layers were washed with brine, dried over anhydrous sodium sulfate, filtered and concentrated under vacuum. The residue was purified by silica gel chromatography (0-50% EA in PE) to afford [3-bromo-4-(trifluoromethyl)phenyl]methanol (3 g, 79.11%) as a white solid. MS (ESI) mass calcd. for C₈H₆BrF₃O, 253.00 m/z, found 252.90 [M−H]⁻.

### Synthesis of (4-(trifluoromethyl)-3-((trimethylsilyl)ethynyl)phenyl)methanol

[3-bromo-4-(trifluoromethyl)phenyl]methanol (3 g, 11.763 mmol), trimethylsilylacetylene (4.62 g, 47.037 mmol), a stir bar and TEA (30 mL) were added to an oven-dried and nitrogen-purged 100 mL vial and stirred until homogenous, then treated with Pd(PPh₃)₂Cl₂ (1.65 g, 2.351 mmol) and CuI (0.9 g, 4.726 mmol) in batches at rt. The resulting mixture was stirred for 48 h at 80° C. under N2 atmosphere, cooled to room temperature, diluted with water and extracted with DCM (30 mL×3). The combined organic layers were washed with brine, dried over anhydrous sodium sulfate, filtered and concentrated under vacuum. The residue was purified by silica gel chromatography (0% to 60% EA in PE) to afford a crude product, which was further purified by reverse-phase chromatography (0%-100% ACN/0.1% FA water) to afford [4-(trifluoromethyl)-3-[2-(trimethylsilyl)ethynyl]phenyl]methanol (1.1 g, 34.34%) as a white solid. MS (ESI) mass calcd. for C₁₃H₁₅F₃OSi, 272.00 m/z, found 271.00 [M−H]⁻.

### Synthesis of 4-(trifluoromethyl)-3-((trimethylsilyl)ethynyl)benzyl methanesulfonate

[4-(trifluoromethyl)-3-[2-(trimethylsilyl)ethynyl]phenyl]methanol (500 mg, 1.836 mmol), DCM (5 mL), a stir bar and TEA (500 mg, 4.941 mmol) were added to a 20 mL vial and stirred until homogenous, then treated with methanesulfonyl chloride (316 mg, 2.759 mmol) dropwise at 0° C. The resulting mixture was stirred for 1.5 h at 0° C., then quenched with water. The resulting mixture was extracted with DCM (5 mL×3). The combined organic layers were washed with brine, dried over anhydrous sodium sulfate, filtered and concentrated under vacuum to afford [4-(trifluoromethyl)-3-[2-(trimethylsilyl)ethynyl]phenyl]methyl methanesulfonate (700 mg, crude) as a yellow oil. It was detected by TLC.

### Synthesis of diethyl (4-(trifluoromethyl)-3-((trimethylsilyl)ethynyl)benzyl)phosphonate

[4-(trifluoromethyl)-3-[2-(trimethylsilyl)ethynyl]phenyl]methyl methanesulfonate (700 mg, 1.998 mmol), a stir bar and triethyl phosphite (7 mL) were added to a 20 mL vial and stirred until homogenous. The resulting mixture was stirred for 3 h at 110° C., then concentrated under vacuum and purified by reverse-phase chromatography (0%-70% ACN/10 mM NH₄HCO₃ water) to afford diethyl [4-(trifluoromethyl)-3-[2-(trimethylsilyl)ethynyl]phenyl]methylphosphonate (440 mg, 56.13%) as a yellow oil. MS (ESI) mass calcd. for C₁₇H₂₄F₃O₃PSi, 392.00 m/z, found 393.05 [M−H]⁻.

### Synthesis of tert-butyl (E)-3-(3-ethynyl-4-(trifluoromethyl)styryl)azetidine-1-carboxylate

Diethyl [4-(trifluoromethyl)-3-[2-(trimethylsilyl)ethynyl]phenyl]methylphosphonate (440 mg, 1.121 mmol), a stir bar, THF (5 mL) and tert-butyl 3-formylazetidine-1-carboxylate (206 mg, 1.112 mmol) were added to 20 mL vial and stirred until homogenous, then treated with potassium t-butoxide (188 mg, 1.675 mmol) in batches at rt. The resulting mixture was stirred for 12 h at 25° C., then quenched with water and extracted with DCM (30 mL×3). The combined organic layers were washed with brine, dried over anhydrous sodium sulfate, filtered and concentrated under vacuum. The residue was purified by silica gel chromatography (0% to 20% EA in PE) to afford tert-butyl 3-[(E)-2-[3-ethynyl-4-(trifluoromethyl)phenyl]ethenyl]azetidine-1-carboxylate (285 mg, 72.34%) as a yellow oil. MS (ESI) mass calcd. for C₁₉H₂₀F₃NO₂, 351.00 m/z, found 296.05[M-Boc+H]¹.

### Synthesis of (E)-3-(3-ethynyl-4-(trifluoromethyl)styryl)azetidine hydrochloride

Tert-butyl 3-[(E)-2-[3-ethynyl-4-(trifluoromethyl)phenyl]ethenyl]azetidine-1-carboxylate (285 mg, 0.811 mmol), a stir bar, dioxane (1.5 mL) and 4 M HCl in the dioxane (1.5 mL) were added to an 8 mL vial and stirred until homogenous. The resulting mixture was stirred for 2 h at 25° C., and concentrated under vacuum to afford 3-[(E)-2-[3-ethynyl-4-(trifluoromethyl)phenyl]ethenyl]azetidine hydrochloride (250 mg, 107.13%) as a yellow oil. MS (ESI) mass calcd. for C₁₄H₁₃ClF₃N, 251.00 m/z, found 252.05 [M−H]⁻.

### Synthesis of (E)-1-(3-(3-ethynyl-4-(trifluoromethyl)styryl)azetidin-1-yl)prop-2-en-1-one

(E)-3-(3-ethynyl-4-(trifluoromethyl)styryl)azetidine hydrochloride (240 mg, 0.955 mmol), prop-2-enoyl prop-2-enoate (181 mg, 1.435 mmol), a stir bar and DCM (3 mL) were added to an 8 mL vial and stirred until homogenous, then treated with TEA (483 mg, 4.773 mmol) in batches at rt. The resulting mixture was stirred for 2 h at 25° C. then quenched with water, and extracted with DCM (10 mL×3). The combined organic layers were washed with brine, dried over anhydrous sodium sulfate, filtered and concentrated under vacuum. The residue was separated by Prep-HPLC (Column: XBridge Prep OBD C18 Column, 30*150 mm, 5 μm; Mobile Phase A: Water (10 mmol/L NH₄HCO₃+0.1% NH₃·H₂O), Mobile Phase B: ACN; Flow rate: 60 mL/min; Gradient: 35% B to 65% B in 7 min, 65% B; Wave Length: 254 nm; RT1(min): 6) to afford 1-{3-[(E)-2-[3-ethynyl-4-(trifluoromethyl)phenyl]ethenyl]azetidin-1-yl}prop-2-en-1-one (25.6 mg) as a yellow oil. MS (ESI) mass calcd. for C₁₇H₁₄F₃NO, 305.00 m/z, found 306.15[M+H]⁺; ¹H NMR (400 MHz, DMSO-d6) δ 7.86-7.81 (m, 1H), 7.78-7.71 (m, 1H), 7.69-7.63 (m, 1H), 6.85 (dd, J=16.0, 8.2 Hz, 1H), 6.60 (d, J=15.9 Hz, 1H), 6.33 (dd, J=17.0, 10.3 Hz, 1H), 6.12 (dd, J=17.0, 2.3 Hz, 1H), 5.68 (dd, J=10.3, 2.3 Hz, 1H), 4.64 (s, 1H), 4.52-4.43 (m, 1H), 4.21-4.09 (m, 2H), 3.90-3.82 (m, 1H), 3.57-3.42 (m, 1H); ¹⁹F NMR (376 MHz, DMSO-d6) δ −60.51.

### Example 46: Synthesis of 1-(7-(4-(trifluoromethyl)phenyl)-2-azaspiro[3.5]non-6-en-2-yl)prop-2-en-1-one

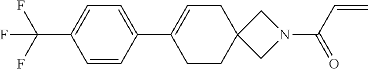 [SMILES: C=CC(=O)N1CC2(CC=C(c3ccc(C(F)(F)F)cc3)CC2)C1]

### Synthetic Route:

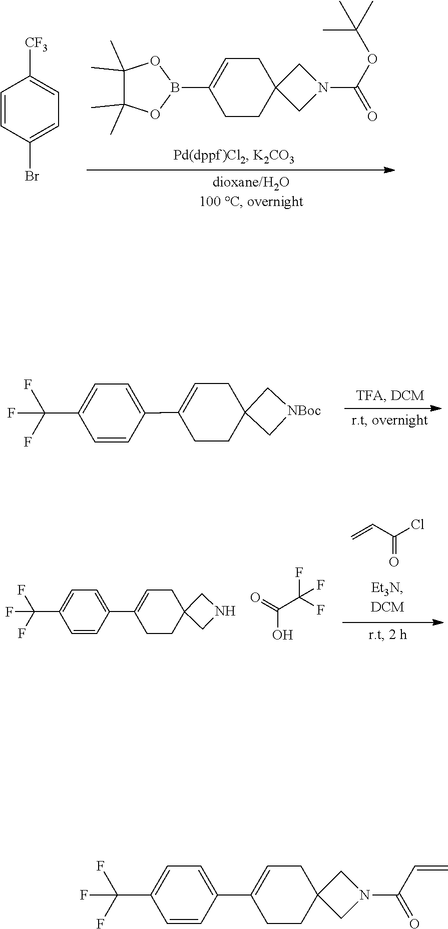

### Synthesis of tert-butyl 7-(4-(trifluoromethyl)phenyl)-2-azaspiro[3.5]non-6-ene-2-carboxylate

To a solution of tert-butyl 7-(4,4,5,5-tetramethyl-1,3,2-dioxaborolan-2-yl)-2-azaspiro[3.5]non-6-ene-2-carboxylate (110 mg, 0.315 mmol) in dioxane (1.5 mL) and H₂O (0.3 mL) was added 1-bromo-4-(trifluoromethyl)benzene (156 mg, 0.693 mmol), potassium carbonate (132 mg, 0.945 mmol) and Pd(dppf)Cl₂ (23 mg, 0.032 mmol). The resulting mixture was maintained under nitrogen and stirred at 100° C. overnight. The resulting mixture was quenched with H₂O and extracted with EA. The organic layers were combined, dried over Na₂SO₄, filtered and concentrated. The residue obtained was purified by silica gel chromatography (EA/PE=0~15%) to afford tert-butyl 7-[4-(trifluoromethyl)phenyl]-2-azaspiro[3.5]non-6-ene-2-carboxylate (107 mg, 92.47%). MS (ESI) mass calcd. for C₂₀H₂₄F₃NO₂, 367.18 m/z, found, 312.05 [M-56+H]⁺.

### Synthesis of 7-(4-(trifluoromethyl)phenyl)-2-azaspiro[3.5]non-6-ene 2,2,2-trifluoroacetate

To a solution of tert-butyl 7-[4-(trifluoromethyl)phenyl]-2-azaspiro[3.5]non-6-ene-2-carboxylate (30 mg, 0.082 mmol) in DCM (1 mL) was added TFA (0.2 mL). The resulting mixture was stirred at r.t. overnight. The resulting mixture was concentrated to afford 7-(4-(trifluoromethyl)phenyl)-2-azaspiro[3.5]non-6-ene 2,2,2-trifluoroacetate (29 mg, 97.22%) as a brown oil. MS (ESI) mass calcd. for C₁₇H₁₇F₆NO, 365.12, found, 268.10 [M-TFA+H]+.

### Synthesis of 1-(7-(4-(trifluoromethyl)phenyl)-2-azaspiro[3.5]non-6-en-2-yl)prop-2-en-1-one

To a solution of 7-(4-(trifluoromethyl)phenyl)-2-azaspiro[3.5]non-6-ene 2,2,2-trifluoroacetate (29 mg, 0.079 mmol) in DCM (0.5 mL) was added triethylamine (48 mg, 0.474 mmol) and acryloyl chloride (8 mg, 0.095 mmol) in DCM (0.5 mL) at r.t. The resulting mixture was stirred at r.t. for 2 h. The reaction was quenched with H₂O and extracted with DCM. The organic layers were combined, dried over Na₂SO₄, filtered and concentrated. The residue obtained was purified by Prep-HPLC (Column: XBridge Prep Phenyl OBD Column, 19*250 mm, 5Î¼ μm; Mobile Phase A: Water (10 mmol/L NH₄HCO₃+0.1% NH₃·H₂O), Mobile Phase B: ACN; Flow rate: 25 mL/min; Gradient: 50% B to 80% B in 7 min, 80% B; Wave Length: 254 nm; RT1(min): 5) to afford 1-{7-[4-(trifluoromethyl)phenyl]-2-azaspiro[3.5]non-6-en-2-yl}prop-2-en-1-one (3.0 mg, 11.73%) as an off-white solid. MS (ESI) mass calcd. for C₁₈H₁₈F₃NO, 321.13, found, 322.15 [M+H]⁺; ¹H NMR (400 MHz, DMSO-d₆) δ 7.54-7.79 (m, 4H), 6.22-6.41 (m, 2H), 6.11 (dd, J=17.0, 2.3 Hz, 1H), 5.66 (dd, J=10.3, 2.3 Hz, 1H), 3.88-4.06 (m, 2H), 3.59-3.77 (m, 2H), 2.45-2.50 (m, 4H), 1.84-2.00 (m, 2H); ¹⁹F NMR (376 MHz, DMSO-d6) δ (ppm): −60.80.

### Example 47: Synthesis of 1-(6-(4-(trifluoromethyl)phenyl)-2-azaspiro[3.4]oct-5-en-2-yl)prop-2-en-1-one

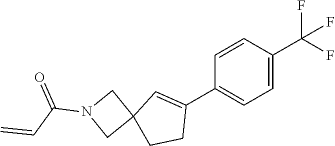 [SMILES: C=CC(=O)N1CC2(C=C(c3ccc(C(F)(F)F)cc3)CC2)C1]

### Synthetic Route:

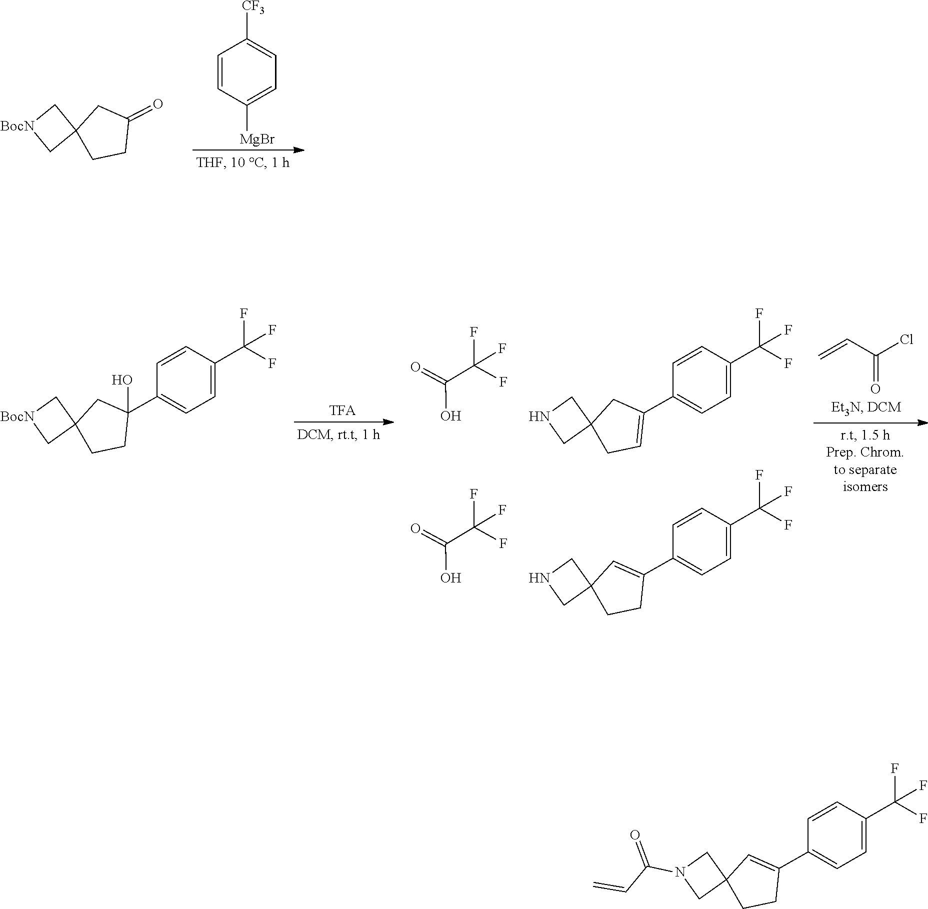

### Synthesis of tert-butyl 6-hydroxy-6-(4-(trifluoromethyl)phenyl)-2-azaspiro[3.4]octane-2-carboxylate

To a solution of tert-butyl 6-oxo-2-azaspiro[3.4]octane-2-carboxylate (1.5 g, 6.658 mmol) in THF (16 mL) was added bromo[4-(trifluoromethyl)phenyl]magnesium (11 mL, 6.658 mmol) at −10° C. under nitrogen atmosphere. The resulting mixture was stirred at −10° C. for 1 h. The reaction was quenched with NH₄Cl solution and extracted with EA. The organic layers were combined, dried over Na₂SO₄, filtered and concentrated. The residue obtained was purified by reverse phase chromatography on C18 (MeCN/H₂O (10 Mm NH₄HCO₃)=5-65%) to afford tert-butyl 6-hydroxy-6-(4-(trifluoromethyl)phenyl)-2-azaspiro[3.3]heptane-2-carboxylate as a yellow oil (1.0 g, 43.11%). MS (ESI) mass calcd. for C₁₉H₂₄F₃NO₃, 371.17, found, 316.10 [M-56+H]⁺.

### Synthesis of 6-(4-(trifluoromethyl)phenyl)-2-azaspiro[3.4]octene 2,2,2-trifluoroacetate

Tert-butyl 6-hydroxy-6-[4-(trifluoromethyl)phenyl]-2-azaspiro[3.4]octane-2-carboxylate (500 mg, 1.35 mmol), a stir bar and DCM (10 mL) and TFA (0.5 mL) were added to a 50 mL flask. The resulting mixture was stirred at r.t. for 1 h. The resulting mixture was concentrated to afford a mixture of 6-[4-(trifluoromethyl)phenyl]-2-azaspiro[3.4]oct-5-ene trifluoroacetate and of 6-[4-(trifluoromethyl)phenyl]-2-azaspiro[3.4]oct-6-ene trifluoroacetate (160 mg, 0.455 mmol). MS (ESI) mass calcd. for C₁₆H₁₅F₆NO₂, 367.10 m/z, found, 254.00 [M-TFA+H]⁺.

### Synthesis of 1-(6-(4-(trifluoromethyl)phenyl)-2-azaspiro[3.4]oct-5-en-2-yl)prop-2-en-1-one

To a mixture of 6-[4-(trifluoromethyl)phenyl]-2-azaspiro[3.4]oct-5-ene trifluoroacetate and of 6-[4-(trifluoromethyl)phenyl]-2-azaspiro[3.4]oct-6-ene trifluoroacetate (160 mg, 0.455 mmol) in DCM (4 mL) was added triethylamine (276 mg, 2.730 mmol) and acryloyl chloride (49 mg, 0.546 mmol) in DCM (1 mL) at r.t. The resulting mixture was stirred at r.t. for 1.5 h. The reaction was quenched with H₂O and extracted with DCM. The organic layers were combined, dried over Na₂SO₄, filtered and concentrated. The residue obtained was purified by Prep-HPLC (Column: Viridis BEH Prep 2-EP OBD Column, 30*150 mm, 5Î¼μm; Mobile Phase A: Hex—HPLC, Mobile Phase B: THF—HPLC; Flow rate: 30 mL/min; Gradient: 5% B to 60% B in 6 min, 60% B; Wave Length: 254 nm; RT1(min): 5.15) to afford 1-(6-(4-(trifluoromethyl)phenyl)-2-azaspiro[3.4]oct-5-en-2-yl)prop-2-en-1-one (4.8 mg) as a white solid. MS (ESI) mass calcd. for C₁₇H₁₆F₃NO, 307.12 m/z, found, 308.05 [M+H]¹; ¹H NMR (300 MHz, DMSO-d₆) δ 7.59-7.84 (m, 4H), 6.61-6.74 (m, 1H), 6.34 (dd, J=17.0, 10.2 Hz, 1H), 6.12 (dd, J=17.0, 2.4 Hz, 1H), 5.68 (dd, J=10.2, 2.4 Hz, 1H), 4.16-4.38 (m, 2H), 3.84-4.09 (m, 2H), 2.63-2.87 (m, 2H), 2.32 (t, J=7.1 Hz, 2H); ¹⁹F NMR (282 MHz, DMSO-d₆) δ (ppm): −60.89.

### Example 48: Synthesis of 1-(6-(4-(trifluoromethyl)phenyl)-2-azaspiro[3.4]oct-6-en-2-yl)prop-2-en-1-one

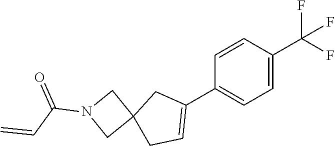 [SMILES: C=CC(=O)N1CC2(CC=C(c3ccc(C(F)(F)F)cc3)C2)C1]

### Synthetic Route:

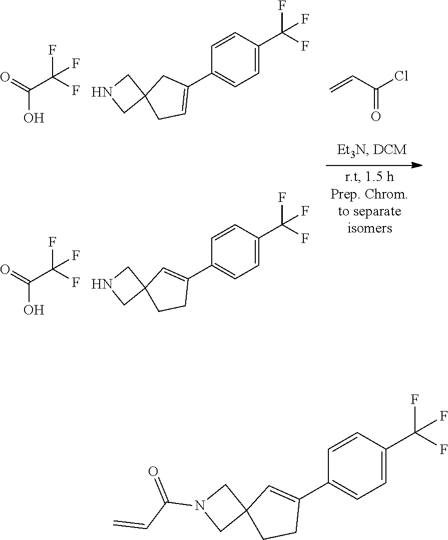

### Synthesis of 1-(6-(4-(trifluoromethyl)phenyl)-2-azaspiro[3.4]oct-6-en-2-yl)prop-2-en-1-one

To a mixture of 6-[4-(trifluoromethyl)phenyl]-2-azaspiro[3.4]oct-5-ene trifluoroacetate and of 6-[4-(trifluoromethyl)phenyl]-2-azaspiro[3.4]oct-6-ene trifluoroacetate (160 mg, 0.455 mmol) in DCM (4 mL) was added triethylamine (276 mg, 2.730 mmol) and acryloyl chloride (49 mg, 0.546 mmol) in DCM (1 mL) at r.t. The resulting mixture was stirred at r.t. for 1.5 h. The reaction was quenched with H₂O and extracted with DCM. The organic layers were combined, dried over Na₂SO₄, filtered and concentrated. The residue obtained was purified by Prep-HPLC (Column: Viridis BEH Prep 2-EP OBD Column, 30*150 mm, 5Î¼μm; Mobile Phase A: Hex—HPLC, Mobile Phase B: THF—HPLC; Flow rate: 30 mL/min; Gradient: 5% B to 60% B in 6 min, 60% B; Wave Length: 254 nm; RT1(min): 5.44) to afford 1-(6-(4-(trifluoromethyl)phenyl)-2-azaspiro[3.4]oct-5-en-2-yl)prop-2-en-1-one (4.9 mg) as a white solid. MS (ESI) mass calcd. for C₁₇H₁₆F₃NO, 307.12 m/z, found, 308.10 [M+H]⁺; ¹H NMR (300 MHz, DMSO-d₆) δ 7.58-7.79 (m, 4H), 6.40-6.48 (m, 1H), 6.33 (dd, J=17.0, 10.2 Hz, 1H), 6.11 (dd, J=17.0, 2.4 Hz, 1H), 5.67 (dd, J=10.2, 2.4 Hz, 1H), 4.07-4.33 (m, 2H), 3.83-4.02 (m, 2H), 3.03 (d, J=2.3 Hz, 2H), 2.84 (s, 2H); ¹⁹F NMR (282 MHz, DMSO-d₆) δ (ppm): −60.84.

### Example 49: Synthesis of 1-{3-[6-(trifluoromethyl)-1-benzofuran-2-yl]azetidin-1-yl}prop-2-en-1-one)

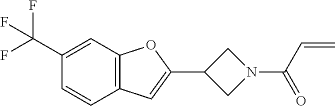 [SMILES: C=CC(=O)N1CC(c2cc3ccc(C(F)(F)F)cc3o2)C1]

### Synthetic Route:

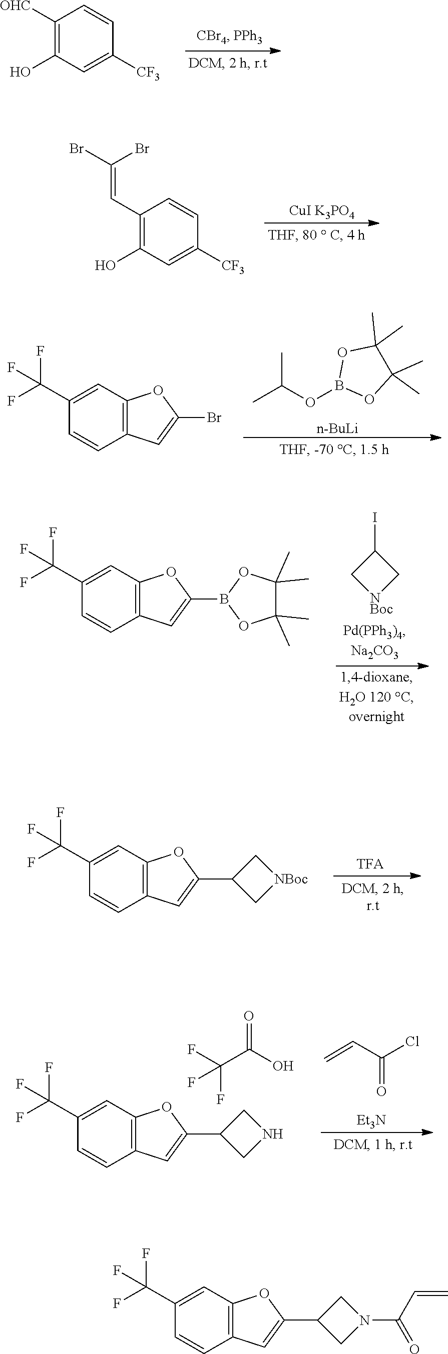

### Synthesis of 2-(2,2-dibromoethenyl)-5-(trifluoromethyl)phenol

2-hydroxy-4-(trifluoromethyl)benzaldehyde (2 g, 10.520 mmol), a stir bar, carbon tetrabromide (10.47 g, 31.572 mmol) and DCM (30 mL) were added to an oven-dried and nitrogen-purged 250 mL round-bottomed flask. The reaction mixture was stirred for 20 min at 0° C., then treated with a solution of triphenylphosphine (8.28 g, 31.560 mmol) in DCM (10 mL) over 5 min, and the resulting mixture stirred for 1 h, before increasing the temperature to room temperature and stirring continued for 2 h. The resulting mixture was quenched with NH₄Cl solution, and extracted with DCM (300 mL×2). The combined organic layers were dried over anhydrous sodium sulfate, filtered and concentrated under vacuum. The residue was purified by silica gel chromatography (EA/PE=0~50%) to afford 2-(2,2-dibromoethenyl)-5-(trifluoromethyl)phenol as a yellow liquid. MS (ESI) mass calcd. for C₉H₅Br₂F₃O, 343.9 m/z, found, 342.10 [M−H]⁻

### Synthesis of 2-bromo-6-(trifluoromethyl)-1-benzofuran

2-(2,2-dibromoethenyl)-5-(trifluoromethyl)phenol (2.2 g, 6.359 mmol), a stir bar, copper(I) iodide (0.121 g, 0.635 mmol), K₃PO₄ (2.7 g, 12.720 mmol) and tetrahydrofuran (60 mL) were added to an oven-dried and nitrogen-purged 250 mL round-bottomed flask, and the resulting mixture stirred for 4 h at 80° C., then cooled to r.t, and diluted with water (200 mL), extracted with EA (350 mL×2). The combined extracts were dried over anhydrous sodium sulfate, filtered, and concentrated to dryness. The residue was purified by silica gel chromatography (EA/PE=0~20%) to afford 2-bromo-6-(trifluoromethyl)-1-benzofuran as a yellow liquid. ¹H NMR (300 MHz, DMSO-d₆) δ 8.14-8.02 (m, 1H), 7.83-7.79 (m, 1H), 7.68-7.59 (m, 1H), 7.36-7.25 (m, 1H). ¹⁹F NMR (282 MHz, DMSO-d₆) δ (ppm): −59.56.

### Synthesis of 4,4,5,5-tetramethyl-2-[6-(trifluoromethyl)-1-benzofuran-2-yl]-1,3,2-dioxaborolane

2-bromo-6-(trifluoromethyl)-1-benzofuran (1.22 g, 4.603 mmol), a stir bar and THF (40 mL) were added to an oven-dried and nitrogen-purged 250 mL round-bottomed flask, which was subsequently cooled to −70° C. (dry ice/EtOH), and the reaction mixture stirred until homogeneous, then treated with 2.5 M n-butyl lithium (2.8 mL, 7.000 mmol) and stirred for 1.5 h, before treated with a solution of 2-isopropoxy-4,4,5,5-tetramethyl-1,3,2-dioxaborolane (1.71 g, 9.191 mmol) in THF (10 mL) over 10 min. The resulting mixture was stirred at −70° C. for 1.5 h, then quenched with NH₄Cl solution (100 mL) and extracted with EA (200 mL×2). The combined organic extracts were washed with brine, dried over anhydrous Na₂SO₄, filtered and concentrated under vacuum to afford 4,4,5,5-tetramethyl-2-[6-(trifluoromethyl)-1-benzofuran-2-yl]-1,3,2-dioxaborolane as a yellow oil (1.8 g. crude).

### Synthesis of tert-butyl 3-[6-(trifluoromethyl)-1-benzofuran-2-yl]azetidine-1-carboxylate

4,4,5,5-tetramethyl-2-[6-(trifluoromethyl)-1-benzofuran-2-yl]-1,3,2-dioxaborolane (900 mg, 2.884 mmol), a stir bar, tert-butyl 3-iodoazetidine-1-carboxylate (1.22 g, 4.309 mmol), tetrakis(triphenylphosphine)palladium(0) (333 mg, 0.288 mmol), Na₂CO₃ (917 mg, 8.570 mmol), water (3 mL) and 1,4-dioxane (15 mL) were added to an oven-dried and nitrogen-purged 250 mL round-bottomed flask, and the resulting mixture stirred at 120° C. overnight. The resulting mixture was cooled to r.t, then quenched with water and extracted with EA (200 mL×2). The combined organic extracts were washed with brine, dried over anhydrous Na₂SO₄, filtered and concentrated under vacuum. The residue was purified by silica gel chromatography (EA/PE=0-40%) to afford tert-butyl 3-[6-(trifluoromethyl)-1-benzofuran-2-yl]azetidine-1-carboxylate as a yellow oil. MS (ESI) calcd. for C₁₇H₁₈F₃NO₃: 341.1 m/z, found: 286.00 [M+H-56]⁺.

### Synthesis of 3-(6-(trifluoromethyl)benzofuran-2-yl)azetidine 2,2,2-trifluoroacetate

Tert-butyl 3-[6-(trifluoromethyl)-1-benzofuran-2-yl]azetidine-1-carboxylate (200 mg, 0.586 mmol), astir bar, DCM (10 mL) were added to a 40 mL vial, and stirred until homogeneous, then treated with trifluoroacetic acid (2 mL). The resulting mixture was stirred at r.t. for 2 h, then concentrated under vacuum to afford 3-(6-(trifluoromethyl)benzofuran-2-yl)azetidine 2,2,2-trifluoroacetate as a yellow oil (295 mg, crude). MS (ESI) calcd. for C₁₂H₁₀F₃NO: 241.1 m/z, found: 242.00 [M+H]⁺.

### Synthesis of 1-{3-[6-(trifluoromethyl)-1-benzofuran-2-yl]azetidin-1-yl}prop-2-en-1-one)

3-(6-(trifluoromethyl)benzofuran-2-yl)azetidine 2,2,2-trifluoroacetate (295 mg. 0.830 mmol), a stir bar and triethylamine (420 mg, 4.150 mmol), DCM (10 mL) were added to an oven-dried and nitrogen-purged 100 mL round-bottomed flask, and stirred until homogeneous before charged with acryloyl chloride (112 mg, 1.237 mmol). The reaction mixture stirred at room temperature for 1 h, then diluted with water, extracted with DCM (200 mL×2). The combined extracts were dried over anhydrous sodium sulfate, filtered, and concentrated to dryness. The residue was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 32% B to 62% B in 7 min, 62% B; Wave Length: 254 nm; RT1(min): 6.53) to afford 1-{3-[6-(trifluoromethyl)-1-benzofuran-2-yl]azetidin-1-yl}prop-2-en-1-one) as a yellow oil (44.4 mg). MS (ESI) calcd. for C₁₅H₁₂F₃NO₂: 295.1 m/z, found: 296.10 [M+H]⁺; ¹H NMR (400 MHz, DMSO-d₆) δ 8.04 (s, 1H), 7.84-7.81 (m, 1H), 7.60-7.57 (m, 1H), 7.06 (s, 1H), 6.41-6.33 (m, 1H), 6.19-6.12 (m, 1H), 5.75-5.70 (m, 1H), 4.69-4.64 (m, 1H), 4.45-4.32 (m, 2H), 4.24-4.10 (m, 2H), ¹⁹F NMR (282 MHz, DMSO-d₆) δ (ppm): −59.30.

### Example 50: Synthesis of 1-(3-(5-(trifluoromethyl)pyrazolo[1,5-a]pyridin-2-yl)azetidin-1-yl)prop-2-en-1-one

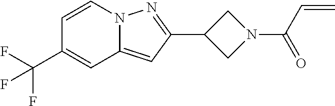 [SMILES: C=CC(=O)N1CC(c2cc3cc(C(F)(F)F)ccn3n2)C1]

### Synthetic Route:

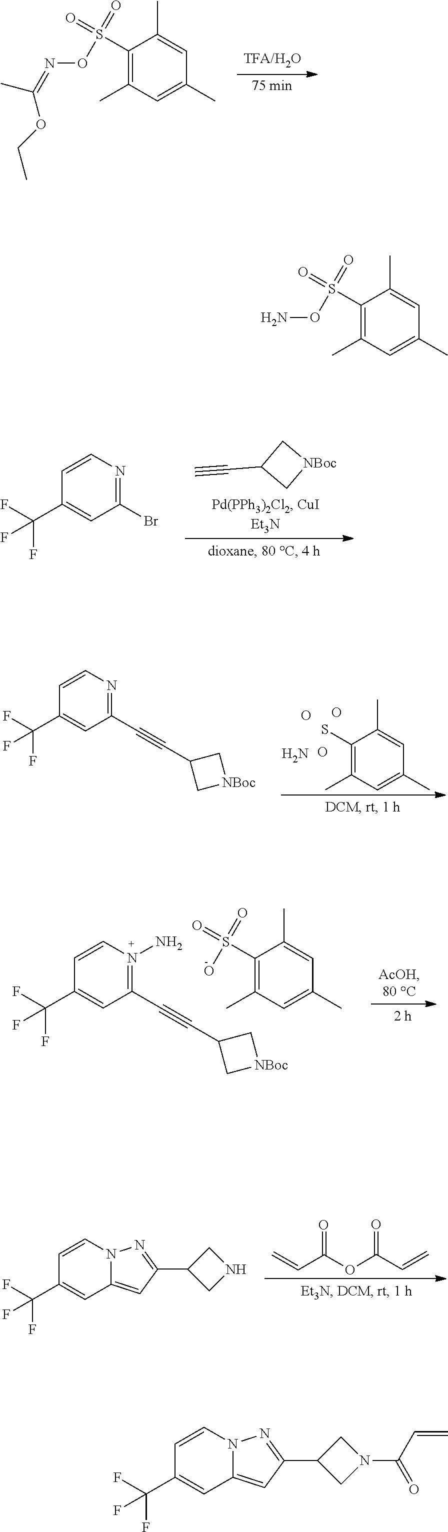

### Synthesis of O-(mesitylsulfonyl)hydroxylamine

Ice water (2.4 mL) and a stir bar were added to a 250 mL three-necked round-bottom flask, then treated with TFA (10 g, 97.91 mmol) dropwise and stirred until homogeneous at 0° C. for 15 min, then treated with (Z)-(ethyl n-[(2,4,6-trimethylbenzenesulfonyl)oxy]ethanimidate) (7.0 g, 24.53 mmol) in portions at 0° C., and stirred for 1 h. Then the resulting mixture was poured into ice water and filtered. The filter cake was dissolved into DCM and dried with anhydrous Na₂SO₄ and filtered to afford a solution of amino 2,4,6-trimethylbenzenesulfonate (3.0 g, crude) in DCM (10 mL) was directly used in the next step immediately. MS (ESI) calcd. for C₉H₁₃NO₃S 215.06 m/z, found 257.10 [M+H+CH₃CN]⁺.

### Synthesis of tert-butyl 3-((4-(trifluoromethyl)pyridin-2-yl)ethynyl)azetidine-1-carboxylate

To a stirred solution of 2-bromo-4-(trifluoromethyl)pyridine (1.0 g, 4.43 mmol) in anhydrous 14-dioxane (15 mL) was added tert-butyl 3-ethynylazetidine-1-carboxylate (963 mg, 5.31 mol), Pd(PPh₃)₂Cl₂ (0.31 g, 0.443 mmol) and CuI (169 mg, 0.887 mmol). Then the resulting mixture was stirred at 80° C. for 4 h under N₂ atmosphere, cooled to room temperature, concentrated and purified by silica gel column (0% to 30% EA in PE) to afford tert-butyl 3-{2-[4-(trifluoromethyl)pyridin-2-yl]ethynyl}azetidine-1-carboxylate (1.1 g, 76.18%) as a light yellow solid. MS (ESI) calcd. for C₁₆H₁₇F₃N₂O₂ 326.12 m/z, found 327.15 [M+H]⁺.

### Synthesis of 1-amino-2-((I-(tert-butoxycarbonyl)azetidin-3-yl)ethynyl)-4-(trifluoromethyl)pyridin-1-ium 2,4,6-trimethylbenzenesulfonate

Tert-butyl 3-{2-[4-(trifluoromethyl)pyridin-2-yl]ethynyl}azetidine-1-carboxylate (500 mg, 1.53 mmol), a stir bar and DCM (5 mL) were added to a 40 mL reaction vessel, then treated with amino 2,4,6-trimethylbenzenesulfonate (1.5 g, 5.97 mmol) in DCM (5 mL) dropwise at room temperature. Then the resulting mixture was stirred at room temperature for 2 h. MTBE (100 mL) was poured into the solution and filtered, the filtrate was concentrated under vacuum pressure to afford 1-amino-2-{2-[1-(tert-butoxycarbonyl)azetidin-3-yl]ethynyl}-4-(trifluoromethyl)pyridin-1-ium (400 mg, crude) as a brown yellow oil which was used in the next step without purification. MS (ESI) calcd. for C₁₆H₁₉F₃N₃O₂ 342.14 m/z, found 342.10 [M+H]⁺.

### Synthesis of 2-(azetidin-3-yl)-5-(trifluoromethyl)pyrazolo[1,5-a]pyridine

1-amino-2-{2-[1-(tert-butoxycarbonyl)azetidin-3-yl]ethynyl}-4-(trifluoromethyl)pyridin-1-ium (400 mg, 1.17 mmol), a stir bar and HOAc (3 mL) were added to a 20 mL vial. Then the resulting mixture was stirred at 80° C. for 2 h, cooled to room temperature and concentrated under vacuum pressure. The obtained residue was purified by C18 (0% to 50% CH₃CN/10 mM NH₄HCO₃ water) to afford 2-(azetidin-3-yl)-5-(trifluoromethyl)pyrazolo[1,5-a]pyridine as a dark yellow solid. MS (ESI) calcd. for C₁₁H₁₀F₃N₃ 241.08 m/z, found 242.05 [M+H]⁺.

### Synthesis of 1-(3-(5-(trifluoromethyl)pyrazolo[1,5-a]pyridin-2-yl)azetidin-1-yl)prop-2-en-1-one

To a stirred solution of 3-[5-(trifluoromethyl)pyrazolo[1,5-a]pyridin-2-yl]azetidine (300 mg, 1.24 mmol) in DCM (5.0 mL) was added Et₃N (400 mg, 3.95 mmol) and prop-2-enoyl prop-2-enoate (320 mg, 2.54 mmol). The reaction mixture was stirred at r.t. for a period of 1 h, diluted with water and extracted with DCM (3×10 mL). The combined organic layer were washed with brine, dried over anhydrous sodium sulfate and concentrated under reduced pressure to give crude product which was further purified by Prep-HPLC (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 25% B to 55% B in 7 min, 55% B; Wave Length: 254 nm; RT1(min): 6.12) to afford 1-{3-[5-(trifluoromethyl)pyrazolo[1,5-a]pyridin-2-yl]azetidin-1-yl}prop-2-en-1-one (24.1 mg, 6.51%) as a brown yellow solid. MS (ESI) calcd. for C₁₄H₂F₃N₃O 295.09 m/z, found 296.10 [M+H]⁺; ¹H NMR (400 MHz, DMSO-d₆) δ 8.85 (d, J=7.2 Hz, 1H), 8.19 (d, J=2.0 Hz, 1H), 7.09 (dd, J=7.3, 2.1 Hz, 1H), 6.90 (s, 1H), 6.36 (dd, J=17.0, 10.3 Hz, 1H), 6.14 (dd, J=17.0, 2.2 Hz, 1H), 5.70 (dd, J=10.3, 2.3 Hz, 1H), 4.71-4.64 (m, 1H), 4.44-4.33 (m, 2H), 4.19-4.04 (m, 2H). ¹⁹F NMR (376 MHz, DMSO-d₆) δ (ppm): −62.05.

### Example 51: Synthesis of 1-(3-(6-(trifluoromethyl)pyrazolo[1,5-a]pyridin-2-yl)azetidin-1-yl)prop-2-en-1-one

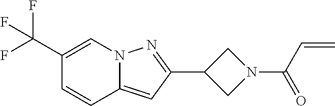 [SMILES: C=CC(=O)N1CC(c2cc3ccc(C(F)(F)F)cn3n2)C1]

### Synthetic Route:

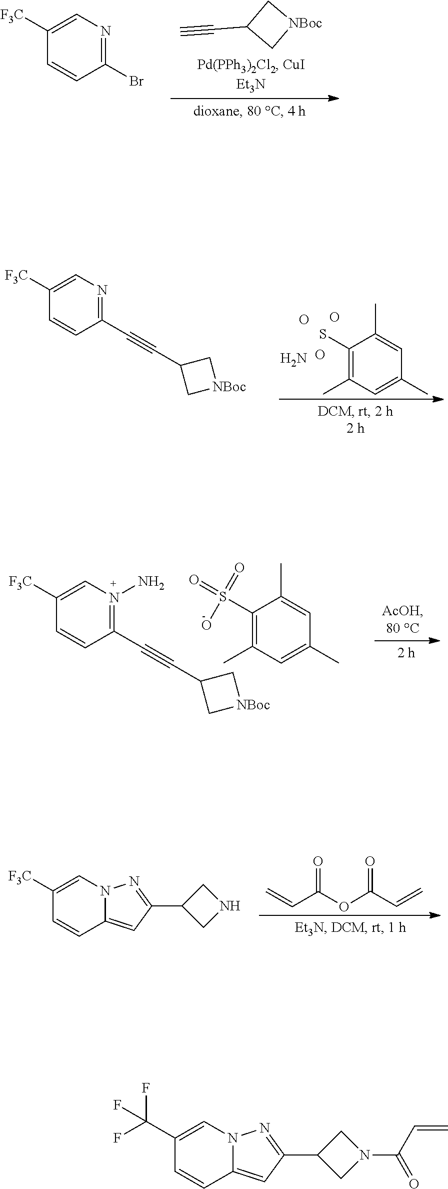

### Synthesis of tert-butyl 3-((5-(trifluoromethyl)pyridin-2-yl)ethynyl)azetidine-1-carboxylate

To a stirred solution of 2-bromo-5-(trifluoromethyl)pyridine (1.0 g, 4.43 mmol) in anhydrous 1,4-dioxane (15 mL) was added tert-butyl 3-ethynylazetidine-1-carboxylate (963 mg, 5.31 mol), Pd(PPh₃)₂Cl₂ (310 mg, 0.442 mmol), CuI (169 mg, 0.887 mmol). Then the resulting mixture was stirred for 4 h at 80° C. under N2 atmosphere, cooled to r.t, and concentrated. The obtained residue was purified by silica gel column (0% to 30% EA in PE) to afford tert-butyl 3-{2-[5-(trifluoromethyl)pyridin-2-yl]ethynyl}azetidine-1-carboxylate (1.2 g, 83.11%) as a reddish brown oil. MS (ESI) calcd. for C₁₆H₁₇F₃N₂O₂ 326.12 m/z, found 327.15 [M+H]⁺.

### Synthesis of 1-amino-2-((1-(tert-butoxycarbonyl)azetidin-3-yl)ethynyl)-5-(trifluoromethyl)pyridin-1-ium 2,4,6-trimethylbenzenesulfonate

Tert-butyl 3-{2-[5-(trifluoromethyl)pyridin-2-yl]ethynyl}azetidine-1-carboxylate (500 mg, 1.53 mmol), a stir bar and DCM (5.0 mL) were added to a 40 mL reaction vessel and stirred until homogeneous, then treated with a solution of amino 2,4,6-trimethylbenzenesulfonate (1.5 g, 5.97 mmol) in DCM (5.0 mL). Then the resulting mixture was stirred at room temperature for 2 h. MTBE (100 mL) was poured into the solution and filtered, the filtrate was concentrated under vacuum pressure to afford 1-amino-2-{2-[1-(tert-butoxycarbonyl)azetidin-3-yl]ethynyl}-5-(trifluoromethyl)pyridin-1-ium (500 mg, crude) as a brown yellow oil which was used in the next step without purification. MS (ESI) calcd. for C₁₆H₁₉F₃N₃O₂ 342.14 m/z, found 342.14 [M+H]⁺.

### Synthesis of 2-(azetidin-3-yl)-6-(trifluoromethyl)pyrazolo[1,5-a]pyridine

1-amino-2-{2-[1-(tert-butoxycarbonyl)azetidin-3-yl]ethynyl}-4-(trifluoromethyl)pyridin-1-ium (500 mg, 1.46 mmol), a stir bar and HOAc (4 mL) were added to a 20 mL vial. The resulting mixture was stirred at 80° C. for 2 h, cooled to r.t, and concentrated. The obtained residue was purified by C18 (0% to 50% CH₃CN/10 mM NH₄HCO₃ water) to afford 3-[6-(trifluoromethyl)pyrazolo[1,5-a]pyridin-2-yl]azetidine as a dark yellow solid. MS (ESI) calcd. for C₁₁H₁₀F₃N₃ 241.08 m/z, found 242.05 [M+H]⁺.

### Synthesis of 1-(3-(6-(trifluoromethyl)pyrazolo[1,5-a]pyridin-2-yl)azetidin-1-yl)prop-2-en-1-one

To a stirred solution of 3-[6-(trifluoromethyl)pyrazolo[1,5-a]pyridin-2-yl]azetidine (150 mg, 0.622 mmol) in DCM (3.0 mL) was added Et₃N (189 mg, 1.87 mmol) and prop-2-enoyl prop-2-enoate (120 mg, 0.95 mmol). The reaction mixture was stirred at r.t. for a period of 1 h, diluted with water 10 mL and extracted with DCM (3×10 mL). The combined organic layers were washed with brine, dried over anhydrous sodium sulfate and concentrated under vacuum pressure to give crude product which was further purified by Prep-HPLC (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 18% B to 48% B in 10 min, 48% B; Wave Length: 254 nm; RT1(min): 8.82) to afford 1-{3-[6-(trifluoromethyl)pyrazolo[1,5-a]pyridin-2-yl]azetidin-1-yl}prop-2-en-1-one (58.3 mg, 31.44%) as a yellow solid. MS (ESI) calcd. for C₁₄H₁₂F₃N₃O 295.09 m/z, found 296.10 [M+H]+; ¹H NMR (400 MHz, DMSO-d₆) δ 9.27 (s, TH), 7.84 (d, J=9.3 Hz, TH), 7.42 (dd, J=9.3, 1.7 Hz, 1H), 6.81 (s, 1H), 6.36 (dd, J=17.0, 10.3 Hz, 1H), 6.14 (dd, J=17.0, 2.2 Hz, 1H), 5.70 (dd, J=10.3, 2.2 Hz, TH), 4.72-4.65 (m, 1H), 4.41-4.33 (m, 2H), 4.18-4.05 (m, 2H). ¹⁹F NMR (376 MHz, DMSO-d₆) δ (ppm): −60.21.

### Example 52: Synthesis of 1-{3-[6-(trifluoromethyl)-1H-indol-2-yl]azetidin-1-yl}prop-2-en-1-one

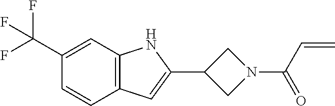 [SMILES: C=CC(=O)N1CC(c2cc3ccc(C(F)(F)F)cc3[nH]2)C1]

### Synthetic Route:

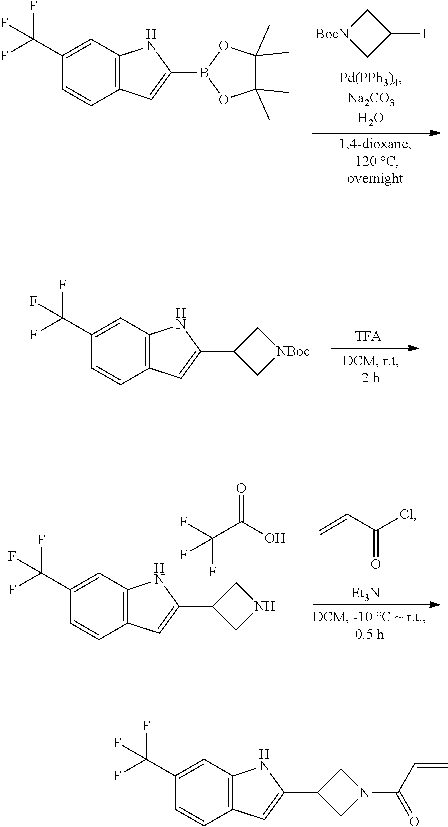

### Synthesis of tert-butyl 3-[6-(trifluoromethyl)-1H-indol-2-yl]azetidine-1-carboxylate

2-(4,4,5,5-tetramethyl-1,3,2-dioxaborolan-2-yl)-6-(trifluoromethyl)-1H-indole (980 mg, 3.150 mmol), tert-butyl 3-iodoazetidine-1-carboxylate (1340 mg, 4.733 mmol), Na₂CO₃ (1000 mg, 9.435 mmol), a stir bar, 1,4-dioxane (15 mL) and H₂O (3 mL) were added to a 40 mL vial, then treated with Pd(PPh₃)₄ (364 mg, 0.315 mmol). The resulting mixture was stirred at 120° C. overnight under a nitrogen atmosphere, then cooled to r.t., diluted with water (20 mL), and extracted with EA (20 mL×2). The combined extracts were washed with brine (40 mL), dried over anhydrous sodium sulfate, filtered, and concentrated to dryness. The residue was subjected to silica gel chromatography (0-30% EA/PE) to afford tert-butyl 3-[6-(trifluoromethyl)-1H-indol-2-yl]azetidine-1-carboxylate as a yellow solid. MS (ESI) mass calcd. for C₁₇H₁₉F₃N₂O₂, 340.14 m/z, found 341.15 [M+H]⁺.

### Synthesis of 2-(azetidin-3-yl)-6-(trifluoromethyl)-1H-indole 2,2,2-trifluoroacetate

Tert-butyl 3-[6-(trifluoromethyl)-1H-indol-2-yl]azetidine-1-carboxylate (514 mg, 1.510 mmol), a stir bar, DCM (15 mL) were added to a 40 mL vial and stirred until homogeneous, then treated with TFA (3 mL). The resulting mixture was stirred at r.t. for 1 h, then concentrated to dryness to afford 2-(azetidin-3-yl)-6-(trifluoromethyl)-1H-indole 2,2,2-trifluoroacetate as a red solid. MS (ESI) mass calcd. for C₁₂H₁₁F₃N₂, 240.09 m/z, found 241.00 [M+H]⁺.

### Synthesis of 1-{3-[6-(trifluoromethyl)-1H-indol-2-yl]azetidin-1-yl}prop-2-en-1-one

2-(azetidin-3-yl)-6-(trifluoromethyl)-1H-indole 2,2,2-trifluoroacetate (653 mg, 1.843 mmol), Et₃N (845 mg, 8.350 mmol), a stir bar, DCM (10 mL) were added to a 40 mL vial and stirred until homogeneous, then treated with acryloyl chloride (127 mg, 1.403 mmol) in DCM (2 mL) at −10° C. under nitrogen atmosphere. The resulting mixture was stirred at r.t. for 30 min, then diluted with water (20 mL), and extracted with DCM (20 mL×2). The combined extracts were dried over anhydrous sodium sulfate, filtered, and concentrated to dryness. The residue was purified by C18 column with ACN/H₂O (0.1% HCOOH) to afford 1-{3-[6-(trifluoromethyl)-1H-indol-2-yl]azetidin-1-yl}prop-2-en-1-one as a white solid (30.3 mg). MS (ESI) mass calcd. for C₁₅H₁₃F₃N₂O, 294.10 m/z, found 295.05 [M+H]⁺. ¹H NMR (400 MHz, DMSO-d₆) δ 11.75 (s, 1H), 7.70-7.62 (m, 2H), 7.30-7.24 (m, 1H), 6.55 (d, J=2.0 Hz, 1H), 6.38 (dd, J=17.0, 10.3 Hz, 1H), 6.15 (dd, J=17.0, 2.3 Hz, 1H), 5.71 (dd, J=10.3, 2.3 Hz, 1H), 4.66 (t, J=8.4 Hz, 1H), 4.40-4.30 (m, 2H), 4.18-4.03 (m, 2H). ¹⁹F NMR (376 MHz, DMSO-d₆) δ (ppm): −60.85.

### Example 53: Synthesis of 1-{3-[I-methyl-6-(trifluoromethyl)indol-2-yl]azetidin-1-yl}prop-2-en-1-one

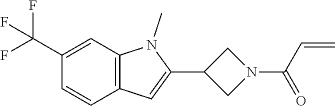 [SMILES: C=CC(=O)N1CC(c2cc3ccc(C(F)(F)F)cc3n2C)C1]

### Synthetic Route:

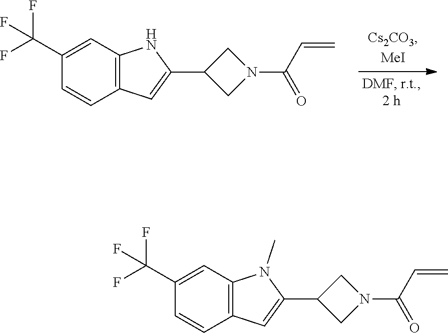 [SMILES: C=CC(=O)N1CC(c2cc3ccc(C(F)(F)F)cc3[nH]2)C1 ; C=CC(=O)N1CC(c2cc3ccc(C(F)(F)F)cc3n2C)C1]

### Synthesis of 1-{3-[1-methyl-6-(trifluoromethyl)indol-2-yl]azetidin-1-yl}prop-2-en-1-one

1-{3-[6-(trifluoromethyl)-1H-indol-2-yl]azetidin-1-yl}prop-2-en-1-one (150 mg, 0.510 mmol), Cs₂CO₃ (249 mg, 0.764 mmol), a stir bar, DMF (10 mL) were added to a 40 mL vial and stirred until homogeneous, then treated with MeI (109 mg, 0.768 mmol). The resulting mixture was stirred at r.t. for 2 h, then diluted with water (30 mL), and extracted with EA (30 mL×2). The combined extracts were washed with brine (60 mL), dried over anhydrous sodium sulfate, filtered, and concentrated to dryness. The residue was purified by Prep HPLC (Column: XBridge Shield RP18 OBD Column, 30*150 mm, 5 μm; Mobile Phase A: Water (10 mmol/L NH₄HCO₃), Mobile Phase B: ACN; Flow rate: 60 mL/min; Gradient: 32% B to 62% B in 7 min, 62% B; Wave Length: 254 nm; RT1(min): 5.7) to afford 1-{3-[1-methyl-6-(trifluoromethyl)indol-2-yl]azetidin-1-yl}prop-2-en-1-one as a white solid. MS (ESI) mass calcd. for C₁₆H₁₅F₃N₂O, 308.11 m/z, found 309.15 [M+H]⁺. ¹H NMR (400 MHz, DMSO-d₆) δ 7.86 (s, 1H), 7.70 (d, J=8.3 Hz, 1H), 7.30 (dd, J=8.2, 1.6 Hz, 1H), 6.68 (s, 1H), 6.37 (dd, J=17.0, 10.3 Hz, 1H), 6.14 (dd, J=17.0, 2.2 Hz, 1H), 5.71 (dd, J=10.3, 2.2 Hz, 1H), 4.73 (t, J=8.6 Hz, 1H), 4.48-4.42 (m, 1H), 4.39-4.34 (m, 1H), 4.31-4.19 (m, 1H), 4.12-4.04 (m, 1H), 3.71 (s, 3H). ¹⁹F NMR (376 MHz, DMSO-d₆) δ (ppm): −58.49.

### Example 54: Synthesis of (E)-1-(4-(3-(trifluoromethyl)styryl)piperidin-1-yl)prop-2-en-1-one

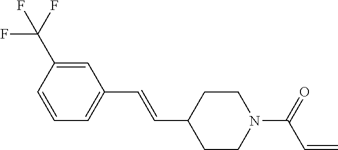 [SMILES: C=CC(=O)N1CCC(/C=C/c2cccc(C(F)(F)F)c2)CC1]

### Synthetic Route:

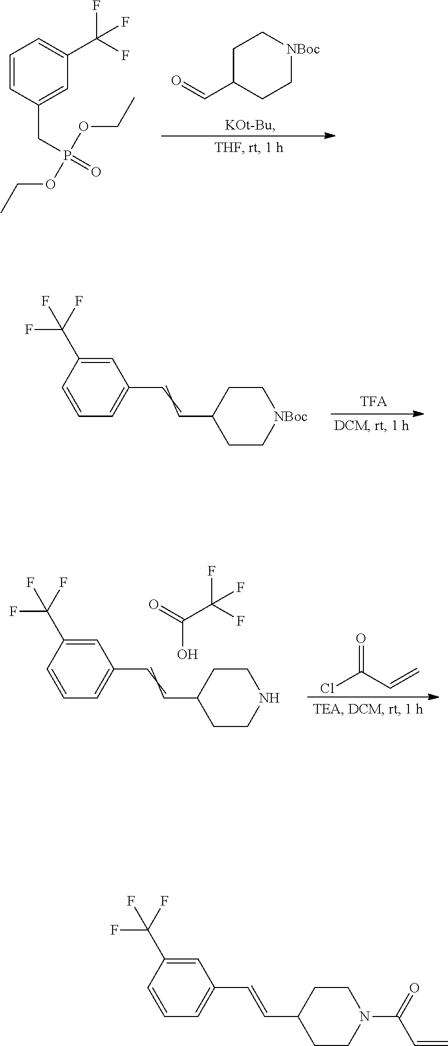

### Synthesis of tert-butyl 4-(3-(trifluoromethyl)styryl)piperidine-1-carboxylate

Diethyl (3-(trifluoromethyl)benzyl)phosphonate (500 mg, 1.688 mmol), a stir bar, tert-butyl 4-formylpiperidine-1-carboxylate (432 mg, 2.026 mmol) and THF (10 mL) were added to a 50 mL round-bottom flask and stirred until homogeneous, and then treated with t-BuOK (284 mg, 2.531 mmol) at 0° C. The resulting mixture was stirred at room temperature for 1 h. The reaction mixture was then quenched with water (20 mL), extracted with EA (3×30 mL), and the combined extracts were dried over anhydrous Na₂SO₄, filtered, and concentrated to dryness in vacuo. The residue obtained was then subjected to silica gel chromatography (0-30% EA/PE) to afford tert-butyl 4-(3-(trifluoromethyl)styryl)piperidine-1-carboxylate as a light yellow oil (539 mg, 89.85%). MS (ESI) calcd. for C₁₉H₂₄F₃NO₂, 355.18 m/z, found 300.05 [M+H-56]⁺.

### Synthesis of 4-(3-(trifluoromethyl)styryl)piperidine 2,2,2-trifluoroacetate

Tert-butyl 4-(3-(trifluoromethyl)styryl)piperidine-1-carboxylate (200 mg, 0.563 mmol), a stir bar, DCM (6 mL) were added to a 50 mL round-bottom flask and stirred until homogeneous, and then treated with TFA (2 mL). The reaction mixture was stirred for 2 h at room temperature, then concentrated under vacuum to afford 4-(3-(trifluoromethyl)styryl)piperidine 2,2,2-trifluoroacetate as a red solid (403 mg, crude). MS (ESI) mass calcd. for C₁₄H₁₆F₃N, 255.12 m/z, found, 256.00 [M+H]⁺.

### Synthesis of (E)-1-(4-(3-(trifluoromethyl)styryl)piperidin-1-yl)prop-2-en-1-one

4-(3-(trifluoromethyl)styryl)piperidine 2,2,2-trifluoroacetate (403 mg, 1.091 mmol), a stir bar, TEA (552 mg, 5.455 mmol) and DCM (6 mL) were added to a 50 mL round-bottom flask and stirred until homogeneous, and then treated with acryloyl chloride (198 mg, 2.188 mmol). The mixture was stirred at room temperature for 1 h, then quenched with water (10 mL) and extracted with DCM (3×20 mL). The combined organic extracts were washed with brine, dried over anhydrous Na₂SO₄, filtered and concentrated under vacuum. The residue was purified by HPLC with (Column: Xselect CSH F-Phenyl OBD column, 19*250 mm, 5 μm; Mobile Phase A: Water (0.1% FA), Mobile Phase B: ACN; Flow rate: 25 mL/min; Gradient: 36% B to 66% B in 10 min, 66% B; Wave Length: 254 nm; RT2 (min): 8.68) to afford (E)-1-(4-(3-(trifluoromethyl)styryl)piperidin-1-yl)prop-2-en-1-one as a light yellow oil (108.9 mg). MS (ESI) mass calcd. for C₁₇H₁₈F₃NO, 309.13 m/z, found, 310.05 [M+H]₊, ¹H NMR (400 MHz, DMSO-d₆) δ 7.80-7.68 (m, 2H), 7.59-7.50 (m, 2H), 6.87-6.78 (m, 1H), 6.57-6.42 (m, 2H), 6.15-6.04 (m, 1H), 5.70-5.62 (m, 1H), 4.48-4.38 (m, 1H), 4.13-4.02 (m, 1H), 3.21-3.06 (m, 1H), 2.82-2.66 (m, 1H), 2.49-2.39 (m, 1H), 1.86-1.73 (m, 2H), 1.38-1.20 (m, 2H). ¹⁹F NMR (376 MHz, DMSO-d₆) δ −61.14.

### Example 55: (Z)-1-(4-(3-(trifluoromethyl)styryl)piperidin-1-yl)prop-2-en-1-one

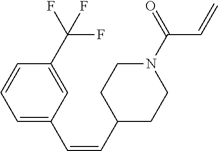 [SMILES: C=CC(=O)N1CCC(/C=C\c2cccc(C(F)(F)F)c2)CC1]

### Synthetic Route:

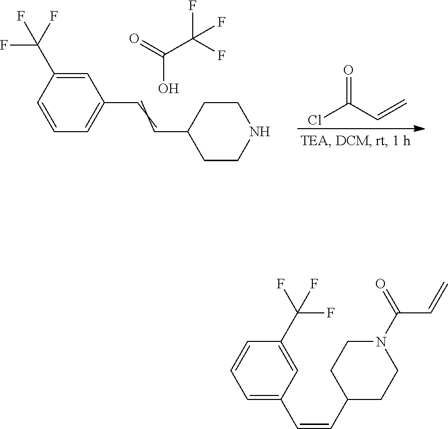

### Synthesis of (Z)-1-(4-(3-(trifluoromethyl)styryl)piperidin-1-yl)prop-2-en-1-one

4-(3-(trifluoromethyl)styryl)piperidine 2,2,2-trifluoroacetate (403 mg, 1.091 mmol), a stir bar, TEA (552 mg, 5.455 mmol) and DCM (6 mL) were added to a 50 mL round-bottom flask and stirred until homogeneous, and then treated with acryloyl chloride (198 mg, 2.188 mmol). The mixture was stirred at room temperature for 1 h, then quenched with water (10 mL) and extracted with DCM (3×20 mL). The combined organic extracts were washed with brine, dried over anhydrous Na₂SO₄, filtered and concentrated under vacuum. The residue was purified by HPLC with (Column: Xselect CSH F-Phenyl OBD column, 19*250 mm, 5 μm; Mobile Phase A: Water (0.1% FA), Mobile Phase B: ACN; Flow rate: 25 mL/min; Gradient: 36% B to 66% B in 10 min, 66% B; Wave Length: 254 nm; RT1(min): 8.20) to afford (Z)-1-(4-(3-(trifluoromethyl)styryl)piperidin-1-yl)prop-2-en-1-one as a light yellow oil (6.9 mg). MS (ESI) mass calcd. for C₁₇H₁₈F₃NO, 309.13 m/z, found, 310.05 [M+H]⁺, ¹H NMR (400 MHz, DMSO-d₆) δ 7.80-7.50 (m, 4H), 6.87-6.75 (m, 1H), 6.49 (d, J=11.7 Hz, 1H), 6.14-6.04 (m, 1H), 5.72-5.57 (m, 2H), 4.47-4.34 (m, 1H), 4.14-3.98 (m, 1H), 3.16-3.00 (m, 1H), 2.82-2.62 (m, 2H), 1.74-1.60 (m, 2H), 1.39-1.15 (m, 2H). ¹⁹F NMR (376 MHz, DMSO-d₆) δ −61.17.

### Example 56: Synthesis of (R*)-1-(3-((3-(trifluoromethyl)phenyl)ethynyl)pyrrolidin-1-yl)prop-2-en-1-one

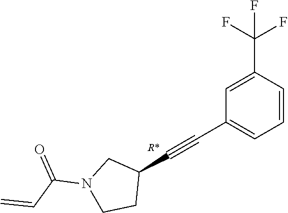 [SMILES: C=CC(=O)N1CC[C@H](C#Cc2cccc(C(F)(F)F)c2)C1]

### Synthetic Route:

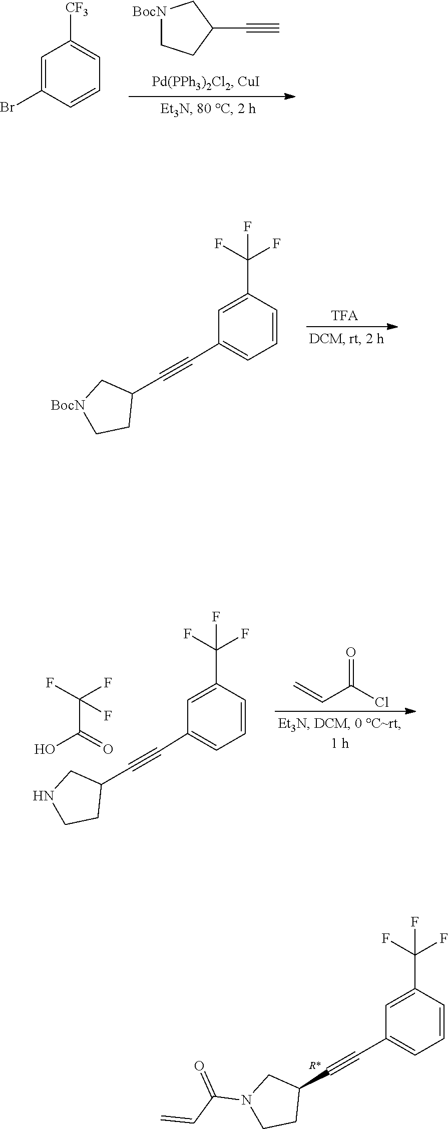

### Synthesis of tert-butyl 3-{2-[3-(trifluoromethyl)phenyl]ethynyl}pyrrolidine-1-carboxylate

1-bromo-3-(trifluoromethyl)benzene (600 mg, 2.667 mmol), triethylamine (12 mL), a stir bar and tert-butyl 3-ethynylpyrrolidine-1-carboxylate (520 mg, 2.663 mmol) were added to a 40 mL vial and stirred until homogeneous, then treated with Pd(PPh₃)₂Cl₂ (375 mg, 0.534 mmol) and copper(I) iodide (102 mg, 0.536 mmol). The resulting mixture was stirred for 2 h at 80° C. under nitrogen atmosphere, then diluted with water (50 mL) and extracted with EA (50 mL×2). The combined extracts were washed with brine, dried over anhydrous sodium sulfate, filtered and concentrated to dryness. The residue was purified by silica gel chromatography (0-20% EA/PE) to afford tert-butyl 3-{2-[3-(trifluoromethyl)phenyl]ethynyl}pyrrolidine-1-carboxylate as a light yellow solid. MS (ESI) mass calcd. for C₁₈H₂₀F₃NO₂, 339.15 m/z, found, 284.00 [M+H-56]⁺.

### Synthesis of 3-((3-(trifluoromethyl)phenyl)ethynyl)pyrrolidine 2,2,2-trifluoroacetate

Tert-butyl 3-{2-[3-(trifluoromethyl)phenyl]ethynyl}pyrrolidine-1-carboxylate (640 mg, 1.886 mmol), DCM (8 mL) and a stir bar were added to a 50 mL round-bottom flask and stirred until homogeneous, then treated with TFA (2 mL). The resulting mixture was stirred for 2 h at r.t, then concentrated under vacuum to afford 3-((3-(trifluoromethyl)phenyl)ethynyl)pyrrolidine 2,2,2-trifluoroacetate as yellow oil (830 mg, crude). MS (ESI) mass calcd. for C₁₃H₁₂F₃N, 239.2 m/z, found, 240.05 [M+H]⁺.

### Synthesis of (R*)-1-(3-((3-(trifluoromethyl)phenyl)ethynyl)pyrrolidin-1-yl)prop-2-en-1-one

3-((3-(trifluoromethyl)phenyl)ethynyl)pyrrolidine 2,2,2-trifluoroacetate (0.83 g, 2.350 mmol), DCM (9 mL), TEA (1.43 g, 14.100 mmol) and a stir bar were added to a 50 mL round-bottom flask and stirred until homogeneous, then treated with prop-2-enoyl chloride (0.28 g, 3.055 mmol) in DCM (1 mL) at 0° C. The resulting mixture was stirred for 1 h at r.t, then diluted with water (100 mL) and extracted with DCM (100 mL×2). The combined extracts were washed with brine, dried over anhydrous sodium sulfate, filtered and concentrated to dryness. The residue was purified by reverse-phase chromatography (5%-70% CH₃CN/10 mM NH₄HCO₃ Water) to afford a racemic product, which was further separated by Prep-Chiral-HPLC with (Column: CHIRALPAK AD-H, 2*25 cm, 5 μm; Mobile Phase A: Hex (0.5% 2M NH3-MeOH)—HPLC. Mobile Phase B: EtOH—HPLC; Flow rate: 20 mL/min; Gradient: 8% B to 8% B in 19 min; Wave Length: 220/254 nm; RT2(min): 16.624; Sample Solvent: EtOH—HPLC; Injection Volume: 0.3 mL) to give two products. The second eluting was lyophilized to afford (absolute stereochemistry unknown, randomly assigns as R) (R*)-1-(3-((3-(trifluoromethyl)phenyl)ethynyl)pyrrolidin-1-yl)prop-2-en-1-one as a colorless oil. MS (ESI) mass calcd. for C₁₆H₁₄F₃NO, 293.1 m/z, found, 294.1 [M+H]⁺. ¹H NMR (400 MHz, DMSO-d6) δ 7.78-7.69 (m, 3H), 7.65-7.59 (m, 1H), 6.65-6.55 (m, 1H), 6.20-6.12 (m, 1H), 5.72-5.66 (m, 1H), 3.81-3.71 (m, 1H), 3.99-3.91, 3.66-3.54 (m, 2H), 3.47-3.35 (m, 2H), 2.35-1.89 (m, 2H).

### Example 57: Synthesis of (S*)-1-(3-((3-(trifluoromethyl)phenyl)ethynyl)pyrrolidin-1-yl)prop-2-en-1-one

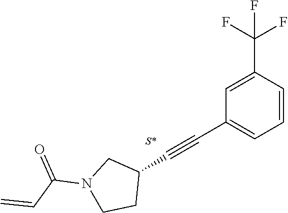 [SMILES: C=CC(=O)N1CC[C@@H](C#Cc2cccc(C(F)(F)F)c2)C1]

### Synthetic Route:

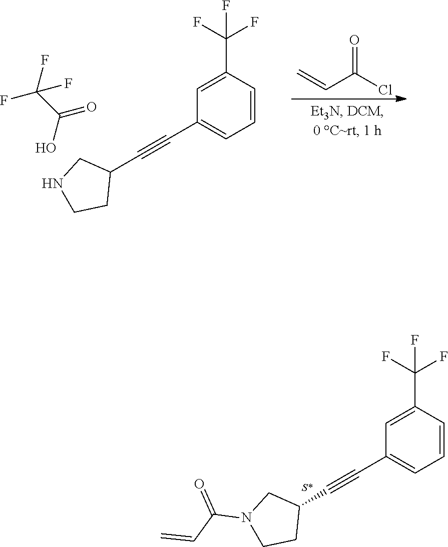

### Synthesis of (S*)-1-(3-((3-(trifluoromethyl)phenyl)ethynyl)pyrrolidin-1-yl)prop-2-en-1-one

3-((3-(trifluoromethyl)phenyl)ethynyl)pyrrolidine 2,2,2-trifluoroacetate (0.83 g, 2.350 mmol), DCM (9 mL), TEA (1.43 g, 14.100 mmol) and a stir bar were added to a 50 mL round-bottom flask and stirred until homogeneous, then treated with prop-2-enoyl chloride (0.28 g, 3.055 mmol) in DCM (1 mL) at 0° C. The resulting mixture was stirred for 1 h at r.t, then diluted with water (100 mL) and extracted with DCM (100 mL×2). The combined extracts were washed with brine, dried over anhydrous sodium sulfate, filtered and concentrated to dryness. The residue was purified by reverse-phase chromatography (5%-70% CH₃CN/10 mM NH₄HCO₃ Water) to afford a racemic product, which was further separated by Prep-Chiral-HPLC with (Column: CHIRALPAK AD-H, 2*25 cm, 5 μm; Mobile Phase A: Hex (0.5% 2M NH3-MeOH)—HPLC, Mobile Phase B: EtOH—HPLC; Flow rate: 20 mL/min; Gradient: 8% B to 8% B in 19 min; Wave Length: 220/254 nm; RT1(min): 14.698; Sample Solvent: EtOH—HPLC; Injection Volume: 0.3 mL) to give two products. The first eluting was lyophilized to afford (absolute stereochemistry unknown, randomly assigns as S) (S*)-1-(3-((3-(trifluoromethyl)phenyl)ethynyl)pyrrolidin-1-yl)prop-2-en-1-one as a colorless oil. MS (ESI) mass calcd. for C₁₆H₁₄F₃NO, 293.1 m/z, found, 294.1 [M+H]⁺. ¹H NMR (400 MHz, DMSO-d6) δ 7.78-7.69 (m, 3H), 7.65-7.57 (m, 1H), 6.64-6.56 (m, 1H), 6.20-6.12 (m, 1H), 5.74-5.66 (m, 1H), 3.81-3.71 (m, 1H), 4.00-3.90, 3.65-3.54 (m, 2H), 3.47-3.36 (m, 2H), 2.35-1.91 (m, 2H). ¹⁹F NMR (376 MHz, DMSO-d₆) δ −61.45.

### Example 58: Synthesis of (S*)-1-(3-methoxy-3-((4-(trifluoromethyl)phenyl)ethynyl)pyrrolidin-1-yl)prop-2-en-1-one

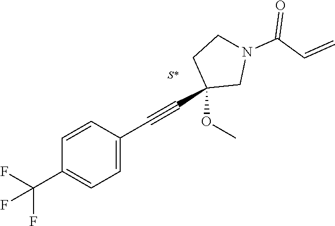 [SMILES: C=CC(=O)N1CC[C@@](C#Cc2ccc(C(F)(F)F)cc2)(OC)C1]

### Synthetic Route:

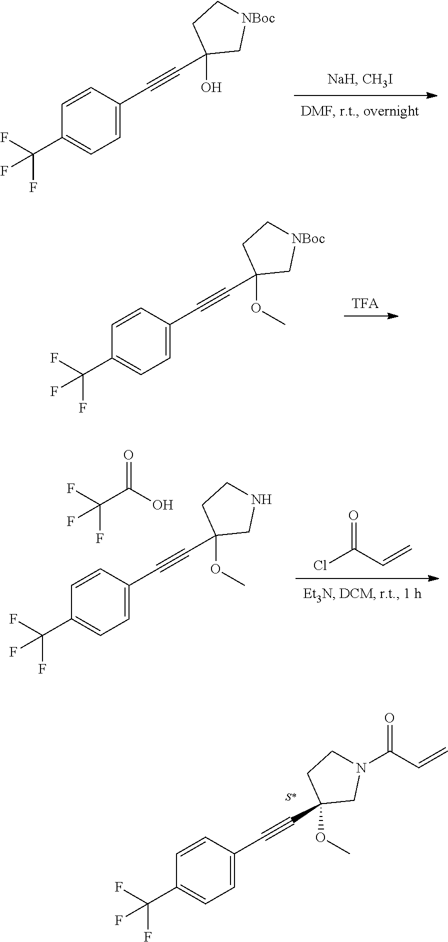

### Synthesis of tert-butyl 3-methoxy-3-((4-(trifluoromethyl)phenyl)ethynyl)pyrrolidine-1-carboxylate

Tert-butyl 3-hydroxy-3-{2-[4-(trifluoromethyl)phenyl]ethynyl}pyrrolidine-1-carboxylate (400 mg, 1.126 mmol, see Example 28), NaH (32.42 mg, 1.351 mmol), a stir bar and dimethylformamide (8 mL) were added to a 20 mL vial and stirred until homogeneous at 0° C. for 30 min, and then treated with CH₃I (191.72 mg, 1.351 mmol) in dimethylformamide (2 mL) dropwise at 0° C. The resulting mixture was stirred overnight at room temperature, then quenched with NH₄Cl. The resulting mixture was extracted with EtOAc (3×30 mL). The combined organic layers were washed with brine (2×10 mL), dried over anhydrous Na₂SO₄, filtered and concentrated under vacuum. The residue was purified by silica gel chromatography (20%-25% EtOAc/PE) to afford tert-butyl 3-methoxy-3-{2-[4-(trifluoromethyl)phenyl]ethynyl}pyrrolidine-1-carboxylate (273 mg, 65.66%) as a yellow oil. MS (ESI) calcd for C₁₉H₂₂F₃NO₃, 369.16 m/z, found, 282.05 [M-MeO⁻-56]⁺.

### Synthesis of 3-methoxy-3-((4-(trifluoromethyl)phenyl)ethynyl)pyrrolidine 2,2,2-trifluoroacetate

Tert-butyl 3-methoxy-3-{2-[4-(trifluoromethyl)phenyl]ethynyl}pyrrolidine-1-carboxylate (200 mg, 0.541 mmol), a stir bar, DCM (5 mL) were added to a 20 mL vial and stirred until homogeneous, and then treated with 2,2,2-trifluoroacetic acid (1.25 mL). The reaction mixture was stirred for 1 h at room temperature under nitrogen atmosphere, then concentrated under vacuum to afford 3-methoxy-3-((4-(trifluoromethyl)phenyl)ethynyl)pyrrolidine 2,2,2-trifluoroacetate (327 mg, crude) as a dark yellow oil. MS (ESI) calcd. for C₁₄H₁₄F₃NO, 269.10 m/z, found, 270.15 [M+H]⁺.

### Synthesis of (S*)-1-(3-hydroxy-3-((4-(trifluoromethyl)phenyl)ethynyl)pyrrolidin-1-yl)prop-2-en-1-one

3-methoxy-3-((4-(trifluoromethyl)phenyl)ethynyl)pyrrolidine 2,2,2-trifluoroacetate (320 mg, 0.871 mmol), triethylamine (440.82 mg, 4.355 mmol), a stir bar, and DCM (8 mL) were added to a 40 mL vial and stirred until homogeneous, then treated with acryloyl chloride (78.86 mg, 0.871 mmol) at 0° C. The resulting mixture was stirred for 1 h at room temperature under nitrogen atmosphere. The reaction mixture was then quenched with water (10 mL), extracted with DCM (3×20 mL). The combined organic layers were washed with brine, dried over anhydrous Na₂ SO₄, filtered and concentrated under vacuum. The residue was purified by reverse-phase chromatography (5%-50% ACN/10 mM NH₄HCO₃ water) to afford a racemic product, which was further separated by Prep-Chiral-HPLC with (Column: CHIRAL ART Cellulose-SB, 2*25 cm, 5 μm; Mobile Phase A: Hex (0.5% 2M NH₃-MeOH)—HPLC, Mobile Phase B: EtOH—HPLC; Flow rate: 20 mL/min; Gradient: 20% B to 20% B in 10 min; Wave Length: 220/254 nm; RT2(min): 8.495; Sample Solvent: EtOH—HPLC; Injection Volume: 0.5 mL) to give two products. The second eluting was lyophilized to afford (absolute stereochemistry unknown, randomly assigned as S) (S*)-1-(3-methoxy-3-((4-(trifluoromethyl)phenyl)ethynyl)pyrrolidin-1-yl)prop-2-en-1-one as light yellow oil (53.4 mg, 18.96%). MS (ESI) calcd. for C₁₇H₁₆F₃NO₂, 323.11 m/z, found, 324.15 [M+H]⁺. ¹H NMR (400 MHz, DMSO-d₆) δ 7.81-7.75 (m, 2H), 7.75-7.69 (m, 2H), 6.65-6.55 (m, 1H), 6.20-6.12 (m, 1H), 5.75-5.65 (m, 1H), 4.04-3.75 (m, 2H), 3.66-3.53 (m, 2H), 3.46-3.37 (m, 3H), 2.47-2.15 (m, 2H). ¹⁹F NMR (376 MHz, DMSO-d₆) δ (ppm): −61.37.

### Example 59: Synthesis of (R*)-1-(3-methoxy-3-((4-(trifluoromethyl)phenyl)ethynyl)pyrrolidin-1-yl)prop-2-en-1-one

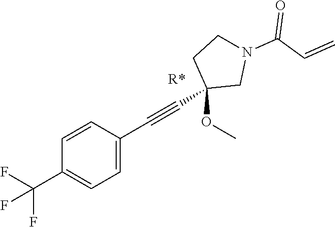 [SMILES: C=CC(=O)N1CC[C@](C#Cc2ccc(C(F)(F)F)cc2)(OC)C1]

### Synthetic Route:

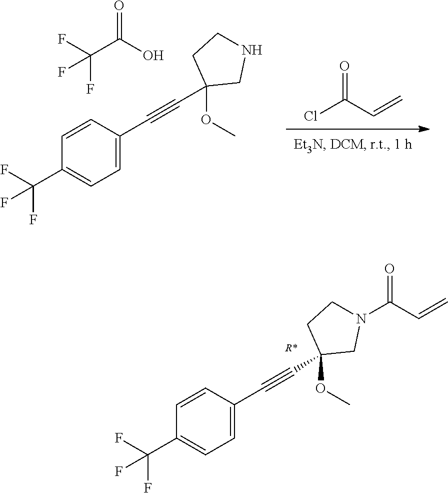

### Synthesis of (R*)-1-(3-methoxy-3-((4-(trifluoromethyl)phenyl)ethynyl)pyrrolidin-1-yl)prop-2-en-1-one

3-methoxy-3-((4-(trifluoromethyl)phenyl)ethynyl)pyrrolidine 2,2,2-trifluoroacetate (320 mg, 0.871 mmol), triethylamine (440.82 mg, 4.355 mmol), a stir bar, and DCM (8 mL) were added to a 40 mL vial and stirred until homogeneous, then treated with acryloyl chloride (78.86 mg, 0.871 mmol) at 0° C. The resulting mixture was stirred for 1 h at room temperature under nitrogen atmosphere. The reaction mixture was then quenched with water (10 mL), extracted with DCM (3×20 mL). The combined organic layers were washed with brine, dried over anhydrous Na₂SO₄, filtered and concentrated under vacuum. The residue was purified by reverse-phase chromatography (5%-50% ACN/10 mM NH₄HCO₃ water) to afford a racemic product, which was further separated by Prep-Chiral-HPLC with (Column: CHIRAL ART Cellulose-SB, 2*25 cm, 5 μm; Mobile Phase A: Hex (0.5% 2M NH₃-MeOH)—HPLC, Mobile Phase B: EtOH—HPLC; Flow rate: 20 mL/min; Gradient: 20% B to 20% B in 10 min; Wave Length: 220/254 nm; RT1(min): 6.987; Sample Solvent: EtOH—HPLC; Injection Volume: 0.5 mL) to give two products. The first eluting was lyophilized to afford (randomly assigned absolute stereochemistry as R) (R*)-1-(3-methoxy-3-((4-(trifluoromethyl)phenyl)ethynyl)pyrrolidin-1-yl)prop-2-en-1-one as a light yellow oil (53.8 mg, 19.10%). MS (ESI) calcd. for C₁₇H₁₆F₃NO₂, 323.11 m/z, found, 324.15 [M+H]⁺. ¹H NMR (400 MHz, DMSO-d₆) δ 7.81-7.75 (m, 2H), 7.75-7.69 (m, 2H), 6.65-6.55 (m, 1H), 6.20-6.12 (m, 1H), 5.74-5.65 (m, 1H), 4.04-3.74 (m, 2H), 3.66-3.53 (m, 2H), 3.45-3.37 (m, 3H), 2.48-2.16 (m, 2H). ¹⁹F NMR (376 MHz, DMSO-d₆) δ (ppm): −61.37.

### Example 60: Synthesis of 1-[(3R*)-3-methyl-3-{2-[4-(trifluoromethyl)phenyl]ethynyl}pyrrolidin-1-yl]prop-2-en-1-one

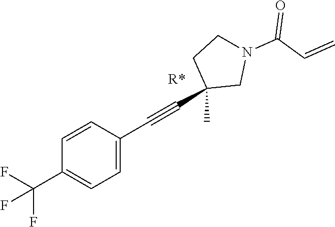 [SMILES: C=CC(=O)N1CC[C@](C)(C#Cc2ccc(C(F)(F)F)cc2)C1]

### Synthetic Route:

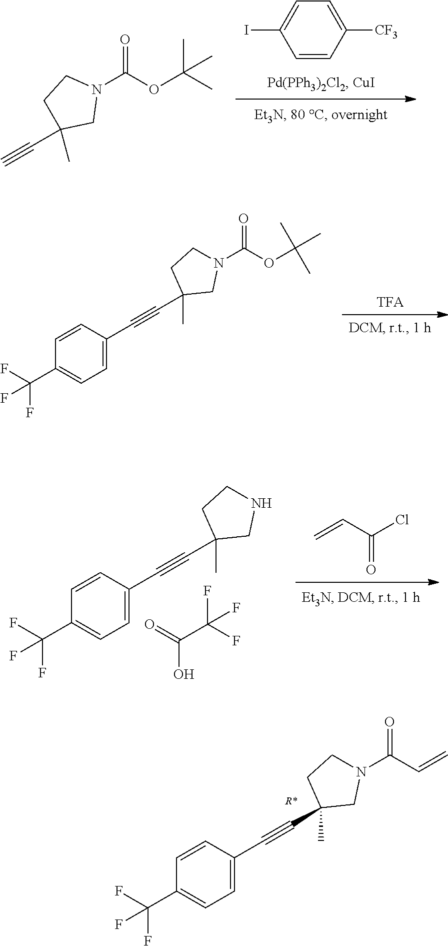

### Synthesis of tert-butyl 3-methyl-3-{2-[4-(trifluoromethyl)phenyl]ethynyl}pyrrolidine-1-carboxylate

Tert-butyl 3-ethynyl-3-methylpyrrolidine-1-carboxylate (250 mg, 1.195 mmol), 1-iodo-4-(trifluoromethyl)benzene (358 mg, 1.316 mmol), a stir bar, and Et₃N (8 mL) were added to a 40 mL vial and stirred until homogeneous, and then treated with CuI (46 mg, 0.242 mmol) and Bis(triphenylphosphine)palladium(II) chloride (168 mg, 0.239 mmol). The resulting mixture was stirred overnight at 80° C. under a nitrogen atmosphere, then cooled to r.t., diluted with H₂O (30 mL), and extracted with EtOAc (30 mL×3), the combined organic layers were dried over Na₂SO₄, filtered and concentrated. The residue was subjected to silica gel chromatography (0-12% EA/PE) to afford tert-butyl 3-methyl-3-{2-[4-(trifluoromethyl)phenyl]ethynyl}pyrrolidine-1-carboxylate as a yellow solid (450 mg, crude). MS (ESI) mass calcd. for C₁₉H₂₂F₃NO₂, 353.16 m/z, found 298.05 [M+H-56]f.

### Synthesis of 3-methyl-3-((4-(trifluoromethyl)phenyl)ethynyl)pyrrolidine 2,2,2-trifluoroacetate

Tert-butyl 3-methyl-3-{2-[4-(trifluoromethyl)phenyl]ethynyl}pyrrolidine-1-carboxylate (450 mg, 1.273 mmol), a stir bar, and DCM (9 mL) were added to a 50 mL round-bottom flask and stirred until homogeneous, and then treated with TFA (3 mL). The resulting mixture was stirred for 1 h at room temperature, then concentrated under vacuum to afford 3-methyl-3-((4-(trifluoromethyl)phenyl)ethynyl)pyrrolidine 2,2,2-trifluoroacetate as a brown oil (690 mg, crude). MS (ESI) calcd. for C₁₄H₁₄F₃N, 253.11 m/z, found 254.05 [M+H]⁺.

### Synthesis of 1-[(3R*)-3-methyl-3-{2-[4-(trifluoromethyl)phenyl]ethynyl}pyrrolidin-1-yl]prop-2-en-1-one

3-methyl-3-((4-(trifluoromethyl)phenyl)ethynyl)pyrrolidine 2,2,2-trifluoroacetate (660 mg, 1.797 mmol), a stir bar, DCM (14.5 mL), and triethylamine (910 mg, 8.993 mmol) were added to a 50 mL round-bottom flask and stirred until homogeneous, then treated with a solution of acryloyl chloride (243 mg, 2.685 mmol) in DCM (0.5 mL) at 0° C. The resulting mixture was stirred at room temperature for 1 h, then diluted with water (50 mL) and extracted with DCM (50 mL×2). The combined extracts were dried over anhydrous sodium sulfate, filtered, and concentrated to dryness. The residue was purified by reverse-phase chromatography (5%-70% ACN/0.1% FA water) to afford a racemic product, which was further separated by Prep_Chiral_HPLC with (Column: Lux 5 um Cellulose-3, 2.12*25 cm, 5 μm; Mobile Phase A: Hex (0.5% 2M NH₃-MeOH)—HPLC, Mobile Phase B: EtOH—HPLC; Flow rate: 20 mL/min; Gradient: 3% B to 3% B in 20 min; Wave Length: 220/254 nm; RT2(min): 17.47; Sample Solvent: EtOH—HPLC; Injection Volume: 0.5 mL) to give two products. The second eluting was lyophilized to afford (stereochemistry randomly assigned as R) 1-[(3R*)-3-methyl-3-{2-[4-(trifluoromethyl)phenyl]ethynyl}pyrrolidin-1-yl]prop-2-en-1-one as alight yellow oil. MS (ESI) calcd. for C₁₇H₁₆F₃NO, 307.12 m/z, found 308.05 [M+H]⁺. ¹H NMR (400 MHz, DMSO-d₆) δ 7.68-7.86 (m, 2H), 7.52-7.68 (m, 2H), 6.27-6.77 (m, 1H), 6.01-6.25 (m, 1H), 5.52-5.92 (m, 1H), 3.64-4.03 (m, 2H), 3.37-3.64 (m, 2H), 2.10-2.30 (m, 1H), 1.90-2.10 (m, 1H), 1.43 (s, 3H). ¹⁹F NMR (376 MHz, DMSO-d6) δ (ppm): −61.05.

### Example 61: Synthesis of 1-[(3S*)-3-methyl-3-{2-[4-(trifluoromethyl)phenyl]ethynyl}pyrrolidin-1-yl]prop-2-en-1-one

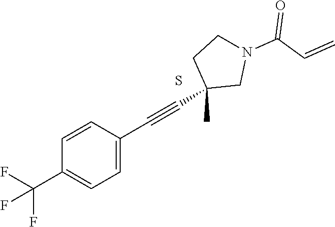

### Synthetic Route:

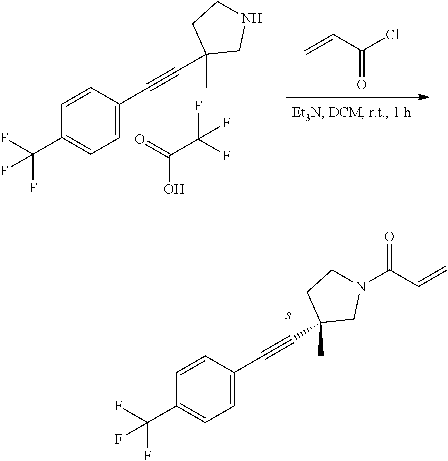

### Synthesis of 1-[(3S)-3-methyl-3-{2-[4-(trifluoromethyl)phenyl]ethynyl}pyrrolidin-1-yl]prop-2-en-1-one

3-methyl-3-((4-(trifluoromethyl)phenyl)ethynyl)pyrrolidine 2,2,2-trifluoroacetate (660 mg, 1.797 mmol), a stir bar, DCM (14.5 mL), and triethylamine (910 mg, 8.993 mmol) were added to a 50 mL round-bottom flask and stirred until homogeneous, then treated with a solution of acryloyl chloride (243 mg, 2.685 mmol) in DCM (0.5 mL) at 0° C. The resulting mixture was stirred at room temperature for 1 h, then diluted with water (50 mL) and extracted with DCM (50 mL×2). The combined extracts were dried over anhydrous sodium sulfate, filtered, and concentrated to dryness. The residue was purified by reverse-phase chromatography (5%-70% ACN/0.1% FA water) to afford a racemic product, which was further separated by Prep_Chiral_HPLC with (Column: Lux 5 um Cellulose-3, 2.12*25 cm, 5 m; Mobile Phase A: Hex (0.5% 2M NH₃-MeOH)—HPLC, Mobile Phase B: EtOH—HPLC; Flow rate: 20 mL/min; Gradient: 3% B to 3% B in 20 min; Wave Length: 220/254 nm; RT1(min): 13.974; Sample Solvent: EtOH—HPLC; Injection Volume: 0.5 mL) to give two products. The first eluting was lyophilized to afford (absolute stereochemistry randomly assigned as S) 1-[(3S*)-3-methyl-3-{2-[4-(trifluoromethyl)phenyl]ethynyl}pyrrolidin-1-yl]prop-2-en-1-one as a light yellow oil (MS (ESI) calcd. for C₁₇H₁₆F₃NO, 307.12 m/z, found 308.05 [M+H]⁺, ¹H NMR (400 MHz, DMSO-d₆) δ 7.68-7.80 (m, 2H), 7.48-7.68 (m, 2H), 6.40-6.76 (m, 1H), 6.00-6.30 (m, 1H), 5.43-5.89 (m, 1H), 3.66-3.98 (m, 2H), 3.45-3.64 (m, 2H), 2.11-2.37 (m, 1H), 1.85-2.09 (m, 1H), 1.43 (s, 3H). ¹⁹F NMR (376 MHz, DMSO-d6) δ (ppm): −61.06).

### Example 62: Synthesis of (S*)-1-(3-fluoro-3-((4-(trifluoromethyl)phenyl)ethynyl)pyrrolidin-1-yl)prop-2-en-1-one

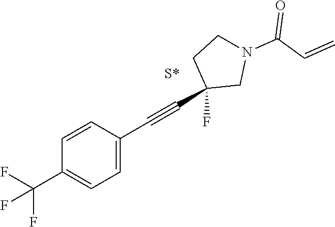 [SMILES: C=CC(=O)N1CC[C@](F)(C#Cc2ccc(C(F)(F)F)cc2)C1]

### Synthetic Route:

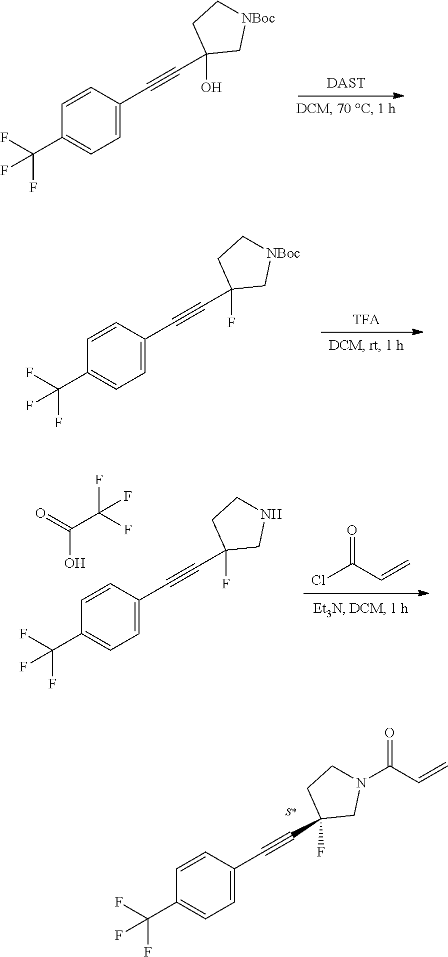

### Synthesis of tert-butyl 3-fluoro-3-((4-(trifluoromethyl)phenyl)ethynyl)pyrrolidine-1-carboxylate

Tert-butyl 3-hydroxy-3-{2-[4-(trifluoromethyl)phenyl]ethynyl}pyrrolidine-1-carboxylate (630 mg, 1.773 mmol), a stir bar, and DCM (15 mL) were added to a 100 mL round-bottom flask and stirred until homogeneous, which was subsequently cooled to −70° C. and slowly charged with DAST (450 mg, 0.174 mmol). The resulting mixture was stirred for 1 h at −70° C., then warmed to −5° C., and stirred for 1 h. The resulting mixture was quenched with NaHCO₃ solution and extracted with DCM (100 mL) twice. The combined extracts were washed with brine, dried over anhydrous sodium sulfate, filtered, and concentrated under vacuum. The residue was subjected to silica gel chromatography (0-30% EA/PE) to afford tert-butyl 3-fluoro-3-{2-[4-(trifluoromethyl)phenyl]ethynyl}pyrrolidine-1-carboxylate as yellow oil. MS (ESI) mass calcd. for C₁₈H₁₉F₄NO₂, 357.14 m/z, found, 301.95 [M-56+H]⁻.

### Synthesis of 3-fluoro-3-((4-(trifluoromethyl)phenyl)ethynyl)pyrrolidine 2,2,2-trifluoroacetate

Tert-butyl 3-fluoro-3-{2-[4-(trifluoromethyl)phenyl]ethynyl}pyrrolidine-1-carboxylate (370 mg, 1.035 mmol), a stir bar and DCM (9 mL) were added to a 100 mL round-bottom flask and stirred until homogeneous, and then treated with TFA (3 mL). The resulting mixture was stirred for 1 h at room temperature, then concentrated under vacuum to afford 3-fluoro-3-((4-(trifluoromethyl)phenyl)ethynyl)pyrrolidine 2,2,2-trifluoroacetate as yellow oil (530 mg, crude). MS (ESI), calcd. for C₁₃H₁₁F₄N, 257.23 m/z, found 258.10 [M+H]⁺.

### Synthesis of (S*)-1-(3-fluoro-3-((4-(trifluoromethyl)phenyl)ethynyl)pyrrolidin-1-yl)prop-2-en-1-one

3-fluoro-3-{2-[4-(trifluoromethyl)phenyl]ethynyl}pyrrolidine 2,2,2-trifluoroacetate (480 mg, 1.293 mmol), triethylamine (655 mg, 6.473 mmol), a stir bar, and DCM (10 mL) were added to a 100 mL round-bottom flask and stirred until homogeneous, then treated with acryloyl chloride (235 mg, 2.596 mmol) at 0° C. The resulting mixture was stirred at r.t. for 1 h, then diluted with water (100 mL) and extracted with DCM (100 mL×2). The combined extracts were washed with brine, dried over anhydrous sodium sulfate, filtered and concentrated under vacuum. The residue was purified by reverse-phase chromatography (0%-45% ACN/10 mM NH₄HCO₃ water) to afford crude product, which was purified by Chiral-HPLC with (Column: CHIRAL ART Cellulose-SB, 2*25 cm, 5 μm; Mobile Phase A: Hex (0.5% 2M NH₃-MeOH)—HPLC, Mobile Phase B: EtOH—HPLC; Flow rate: 20 mL/min; Gradient: 20% B to 20% B in 16 min; Wave Length: 220/254 nm; RT2(min): 13.023; Sample Solvent: EtOH—HPLC; Injection Volume: 0.5 mL) to give two products. The second eluting was lyophilized to afford (absolute stereochemistry unknown, randomly assigns as S) (S*)-1-(3-fluoro-3-((4-(trifluoromethyl)phenyl)ethynyl)pyrrolidin-1-yl)prop-2-en-1-one as a white solid (MS (ESI) mass calcd. for C₁₆H₁₃F₄NO, 311.09 m/z, found, 312.10 [M+H]⁺). ¹H NMR (400 MHz, DMSO-d₆) δ 7.86-7.73 (m, 4H), 6.69-6.53 (m, 1H), 6.23-6.15 (m, 1H), 5.79-5.66 (m, 1H), 4.35-3.88 (m, 2H), 3.85-3.42 (m, 2H), 2.69-2.53 (m, 1H), 2.46-2.33 (m, 1H). ¹⁹F NMR (376 MHz, DMSO-d₆) δ (ppm): −61.47, −138.97. ¹⁹F NMR (376 MHz, DMSO-d) δ −61.42.

### Example 63: Synthesis of Trifluoromethyl)phenyl)ethynyl)pyrrolidin-1-yl)prop-2-en-1-one

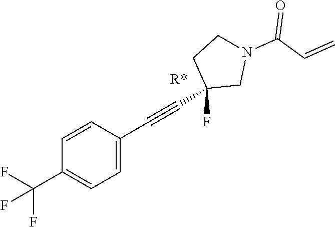 [SMILES: C=CC(=O)N1CC[C@@](F)(C#Cc2ccc(C(F)(F)F)cc2)C1]

### Synthetic Route:

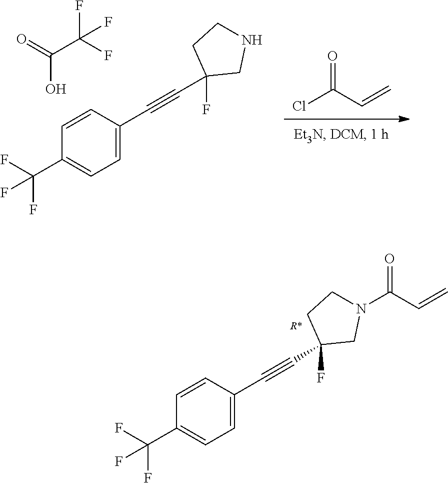

### Synthesis of (R*)-1-(3-fluoro-3-((4-(trifluoromethyl)phenyl)ethynyl)pyrrolidin-1-yl)prop-2-en-1-one

3-fluoro-3-{2-[4-(trifluoromethyl)phenyl]ethynyl}pyrrolidine 2,2,2-trifluoroacetate (480 mg, 1.293 mmol), triethylamine (655 mg, 6.473 mmol), a stir bar, and DCM (10 mL) were added to a 100 mL round-bottom flask and stirred until homogeneous, then treated with acryloyl chloride (235 mg, 2.596 mmol) at 0° C. The resulting mixture was stirred at r.t. for 1 h, then diluted with water (100 mL) and extracted with DCM (100 mL×2). The combined extracts were washed with brine, dried over anhydrous sodium sulfate, filtered and concentrated under vacuum. The residue was purified by reverse-phase chromatography (0%-45% ACN/10 mM NH₄HCO₃ water) to afford a racemic product, which was further separated by Chiral-HPLC with (Column: CHIRAL ART Cellulose-SB, 2*25 cm, 5 μm; Mobile Phase A: Hex (0.5% 2M NH₃-MeOH)—HPLC, Mobile Phase B: EtOH—HPLC; Flow rate: 20 mL/min; Gradient: 20% B to 20% B in 16 min; Wave Length: 220/254 nm; RT1(min): 7.827; Sample Solvent: EtOH—HPLC; Injection Volume: 0.5 mL) to give two products. The first eluting was lyophilized to afford (absolute stereochemistry unknown, randomly assigns as R) (R*)-1-(3-fluoro-3-((4-(trifluoromethyl)phenyl)ethynyl)pyrrolidin-1-yl)prop-2-en-1-one as a white solid (MS (ESI) mass calcd. for C₁₆H₁₃F₄NO, 311.09 m/z, found, 312.10 [M+H]⁺). ¹H NMR (400 MHz, DMSO-d₆) δ 7.86-7.72 (m, 4H), 6.68-6.55 (m, 1H), 6.26-6.13 (m, 1H), 5.79-5.67 (m, 1H), 4.34-3.89 (m, 2H), 3.85-3.42 (m, 2H), 2.66-2.52 (m, 1H), 2.48-2.32 (m, H). ¹⁹F NMR (376 MHz, DMSO-d₆) δ (ppm): −60.25, −138.90.

### Example 64: Synthesis of (S*)-1-(3-hydroxy-3-((4-(trifluoromethyl)phenyl)ethynyl)pyrrolidin-1-yl)prop-2-en-1-one

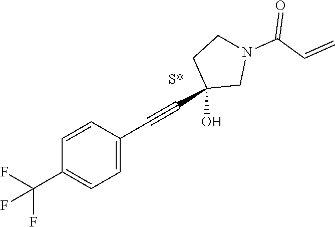 [SMILES: C=CC(=O)N1CC[C@](O)(C#Cc2ccc(C(F)(F)F)cc2)C1]

### Synthetic Route:

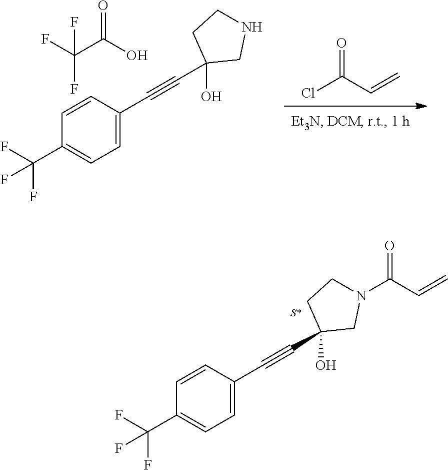

### Synthesis of (S*)-1-(3-hydroxy-3-((4-(trifluoromethyl)phenyl)ethynyl)pyrrolidin-1-yl)prop-2-en-1-one

3-((4-(trifluoromethyl)phenyl)ethynyl)pyrrolidin-3-ol 2,2,2-trifluoroacetate (441 mg, 1.194 mmol), TEA (604 mg, 5.969 mmol), a stir bar and DCM (8 mL) were added to a 50 mL round-bottom flask and stirred until homogeneous, and then treated with acryloyl chloride (108 mg, 1.193 mmol) at 0° C. The resulting mixture was stirred for 1 h at room temperature under nitrogen atmosphere. The reaction mixture was then quenched with water (10 mL), extracted with DCM (3×20 mL). The combined organic layers were washed with brine, dried over anhydrous Na₂SO₄, filtered and concentrated under vacuum. The residue was purified by reverse-phase chromatography (5%-50% ACN/10 mM NH₄HCO₃ water) to afford a racemic product, which was further separated by Prep-Chiral-HPLC with (Column: Lux 5 um Cellulose-4, 2.12*25 cm, 5 μm; Mobile Phase A: Hex (0.5% 2M NH₃-MeOH)—HPLC, Mobile Phase B: IPA—HPLC; Flow rate: 20 mL/min; Gradient: 8% B to 8% B in 35 min; Wave Length: 220/254 nm; RT2(min): 30.151; Sample Solvent: EtOH—HPLC; Injection Volume: 0.5 mL) to give two products. The second eluting was lyophilized to afford (absolute stereochemistry unknown, randomly assigned as S) (S*)-1-(3-hydroxy-3-((4-(trifluoromethyl)phenyl)ethynyl)pyrrolidin-1-yl)prop-2-en-1-one as white solid (87.3 mg, 23.39%). MS (ESI) calcd. for C₁₆H₁₄F₃NO₂, 309.10 m/z, found 310.15 [M+H]⁺. ¹H NMR (400 MHz, DMSO-d₆) δ 7.85-7.72 (m, 2H), 7.72-7.58 (m, 2H), 6.67-6.47 (m, 1H), 6.23-6.15 (m, 1H), 6.15-6.06 (m, 1H), 5.76-5.62 (m, 1H), 3.86-3.69 (m, 2H), 3.68-3.57 (m, 1H), 3.54 (s, 1H), 2.34-2.24 (m, 1H), 2.24-2.11 (m, 1H). ¹⁹F NMR (376 MHz, DMSO-d6) δ (ppm): −61.32

### Example 65: Synthesis of (R*)-1-(3-hydroxy-3-((4-(trifluoromethyl)phenyl)ethynyl)pyrrolidin-1-yl)prop-2-en-1-one

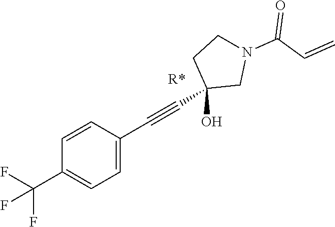 [SMILES: C=CC(=O)N1CC[C@@](O)(C#Cc2ccc(C(F)(F)F)cc2)C1]

### Synthetic Route:

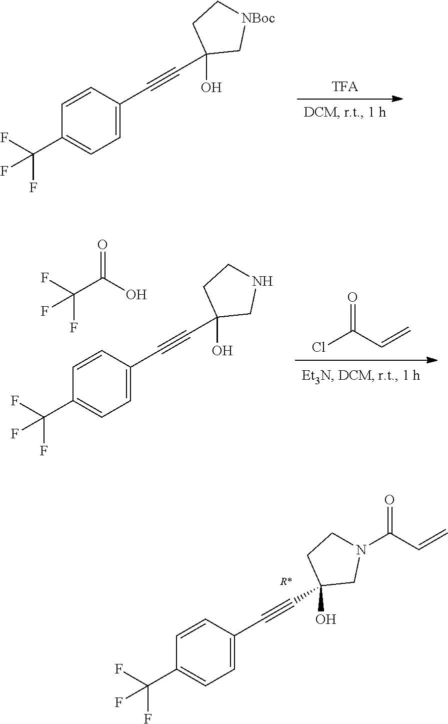

### Synthesis of 3-((4-(trifluoromethyl)phenyl)ethynyl)pyrrolidin-3-ol 2,2,2-trifluoroacetate

Tert-butyl 3-hydroxy-3-{2-[4-(trifluoromethyl)phenyl]ethynyl}pyrrolidine-1-carboxylate (300 mg, 0.844 mmol), a stir bar and DCM (6 mL) were added to a 8 mL vial and stirred until homogeneous, and then treated with 2,2,2-trifluoroacetic acid (1.5 mL). The reaction mixture was stirred for 1 h at room temperature, then concentrated under vacuum to afford 3-((4-(trifluoromethyl)phenyl)ethynyl)pyrrolidin-3-ol 2,2,2-trifluoroacetate (504 mg, crude) as a dark yellow oil. MS (ESI) calcd. for C₁₃H₁₂F₃NO, 255.09 m/z, found, 256.15 [M+H]⁺.

### Synthesis of (R*)-1-(3-hydroxy-3-((4-(trifluoromethyl)phenyl)ethynyl)pyrrolidin-1-yl)prop-2-en-1-one

3-((4-(trifluoromethyl)phenyl)ethynyl)pyrrolidin-3-ol 2,2,2-trifluoroacetate (441 mg, 1.194 mmol), TEA (604 mg, 5.969 mmol), a stir bar and DCM (8 mL) were added to a 50 mL round-bottom flask and stirred until homogeneous, and then treated with acryloyl chloride (108 mg, 1.193 mmol) at 0° C. The resulting mixture was stirred for 1 h at room temperature under nitrogen atmosphere. The reaction mixture was then quenched with water (10 mL), extracted with DCM (3×20 mL). The combined organic layers were washed with brine, dried over anhydrous Na₂SO₄, filtered and concentrated under vacuum. The residue was purified by reverse-phase chromatography (5%-50% ACN/10 mM NH₄HCO₃ water) to afford a racemic product, which was further separated by Prep-Chiral-HPLC with (Column: Lux 5 um Cellulose-4, 2.12*25 cm, 5 μm; Mobile Phase A: Hex (0.5% 2M NH₃-MeOH)—HPLC, Mobile Phase B: IPA—HPLC; Flow rate: 20 mL/min; Gradient: 8% B to 8% B in 35 min; Wave Length: 220/254 nm; RT1(min): 23.98; Sample Solvent: EtOH—HPLC; Injection Volume: 0.5 mL) to give two products. The first eluting was lyophilized to afford (absolute stereochemistry unknown, randomly assigned as R) (R*)-1-(3-hydroxy-3-((4-(trifluoromethyl)phenyl)ethynyl)pyrrolidin-1-yl)prop-2-en-1-one as a white solid (78.8 mg, 21.32%). MS (ESI) calcd. for C₁₆H₁₄F₃NO₂, 309.10 m/z, found, 310.10 [M+H]¹. ¹H NMR (400 MHz, DMSO-d6) δ 7.84-7.72 (m, 2H), 7.72-7.62 (m, 2H), 6.66-6.49 (m, 1H), 6.22-6.07 (m, 2H), 5.74-5.63 (m, 1H), 3.85-3.69 (m, 2H), 3.69-3.57 (m, 1H), 3.57-3.44 (m, 1H), 2.35-2.23 (m, 1H), 2.23-2.13 (m, 1H). ¹⁹F NMR (376 MHz, DMSO-d6) δ (ppm): −61.33.

### Example 66: Synthesis of (R*)-1-(3-((4-(trifluoromethyl)phenyl)ethynyl)pyrrolidin-1-yl)prop-2-en-1-one

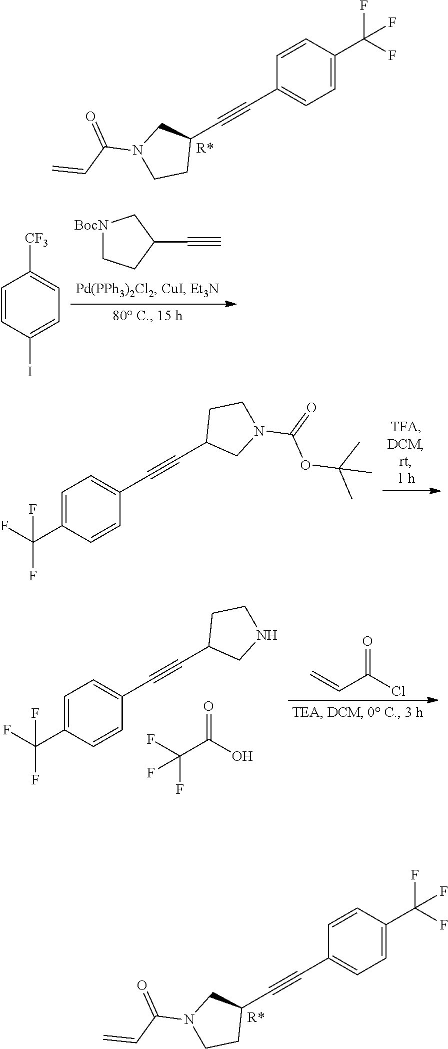

### Synthesis of tert-butyl 3-((4-(trifluoromethyl)phenyl)ethynyl)pyrrolidine-1-carboxylate

1-iodo-4-(trifluoromethyl)benzene (650 mg, 2.390 mmol), a stir bar, TEA (10 mL) and tert-butyl 3-ethynylpyrrolidine-1-carboxylate (466 mg, 2.387 mmol) were added to an oven-dried and nitrogen-purged 25 mL round-bottom flask and stirred until homogenous, then treated with CuI (91 mg, 0.478 mmol) and Pd(PPh₃)₂Cl₂ (335 mg, 0.477 mmol). The resulting mixture was stirred for 15 h at 80° C. under N₂ atmosphere, then cooled to room temperature, quenched with water and extracted with DCM (50 mL×3). The combined organic layers were washed with brine, dried over anhydrous sodium sulfate, filtered and concentrated under vacuum. The residue was purified by silica gel chromatography (0% to 20% EA in PE) to afford tert-butyl 3-{2-[4-(trifluoromethyl)phenyl]ethynyl}pyrrolidine-1-carboxylate (640 mg, 78.92%) as a yellow solid. MS (ESI) mass calcd. for C₁₈H₂₀F₃NO₂, 339.00 m/z, found 325.15[M+H-56+CH₃CN]⁺.

### Synthesis of 3-((4-(trifluoromethyl)phenyl)ethynyl)pyrrolidine 2,2,2-trifluoroacetate

Tert-butyl 3-{2-[4-(trifluoromethyl)phenyl]ethynyl}pyrrolidine-1-carboxylate (640 mg, 1.886 mmol), a stir bar, DCM (6 mL) and TFA (2 mL) were added to a 8 mL vial and stirred until homogenous. The resulting mixture was stirred for 1 h at 25° C. then concentrated under vacuum to afford 3-((4-(trifluoromethyl)phenyl)ethynyl)pyrrolidine 2,2,2-trifluoroacetate (600 mg, 132.98%, crude) as a yellow oil. MS (ESI) mass calcd. for C₁₃H₁₂F₃N, 239.00 m/z, found 240.15[M+H]⁺.

### Synthesis of (R*)-1-(3-((4-(trifluoromethyl)phenyl)ethynyl)pyrrolidin-1-yl)prop-2-en-1-one

3-((4-(trifluoromethyl)phenyl)ethynyl)pyrrolidine 2,2,2-trifluoroacetate (500 mg, 2.090 mmol), a stir bar, DCM (5 mL) and TEA (1056 mg, 10.436 mmol) were add to a 8 mL vial, and stirred until homogeneous, then treated with acryloyl chloride (282 mg, 3.116 mmol) dropwise at rt. The resulting mixture was stirred for 3 h at 0° C., then quenched with water. The resulting mixture was extracted with DCM (50 mL×3). The combined organic layers were washed with brine, dried over anhydrous sodium sulfate, filtered and concentrated under vacuum. The residue was purified by Prep-HPLC (Column: XBridge Prep Phenyl OBD Column, 19*250 mm, 5 μm; Mobile Phase A: Water (10 mmol/L NH₄HCO₃), Mobile Phase B: ACN; Flow rate: 25 mL/min; Gradient: 50% B to 65% B in 9 min, 65% B; Wave Length: 254 nm; RT1(min): 8.23) to afford a racemic product, which was further separated by PREP-CHIRAL-HPLC with (Column: CHIRAL ART Amylose-C NEO, 2*25 cm, 5 μm; Mobile Phase A: Hex (0.5% 2M NH3-MeOH)—HPLC, Mobile Phase B: EtOH—HPLC; Flow rate: 20 m/min; Gradient: 10% B to 10% B in 17 min; Wave Length: 220/254 nm; RT1(min): 13.073; Sample Solvent: EtOH—HPLC; Injection Volume: 0.4 mL) to give two products. The first eluting was lyophilized to afford (absolute stereochemistry unknown, randomly assigned as R) 1-[(3R*)-3-{2-[4-(trifluoromethyl)phenyl]ethynyl}pyrrolidin-1-yl]prop-2-en-1-one (60.7 mg, 9.90%) as a white solid. MS (ESI) calcd. for C₁₆H₁₄F₃NO, 293.00 m/z, found 294.10 [M+H]⁺; ¹H NMR (400 MHz, DMSO-d₆) δ 7.77-7.70 (m, 4H), 7.66-7.59 (m, 4H), 6.67-6.55 (m, 2H), 6.22-6.11 (m, 2H), 5.75-5.64 (m, 2H), 4.01-3.52 (m, 6H), 3.50-3.33 (m, 5H), 2.38-2.18 (m, 2H), 2.14-1.90 (m, 2H); ¹⁹F NMR (376 MHz, DMSO-d6) δ −61.28.

### Example 67: Synthesis of (S*)-1-(3-((4-(trifluoromethyl)phenyl)ethynyl)pyrrolidin-1-yl)prop-2-en-1-one

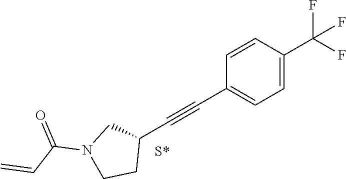 [SMILES: C=CC(=O)N1CC[C@@H](C#Cc2ccc(C(F)(F)F)cc2)C1]

### Synthetic Route:

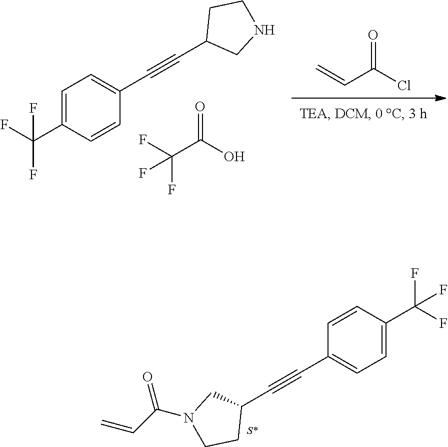

### Synthesis of (S*)-1-(3-((4-(trifluoromethyl)phenyl)ethynyl)pyrrolidin-1-yl)prop-2-en-1-one

3-((4-(trifluoromethyl)phenyl)ethynyl)pyrrolidine 2,2,2-trifluoroacetate (500 mg. 2.090 mmol), a stir bar, DCM (5 mL) and TEA (1056 mg, 10.436 mmol) were add to a 8 mL vial, and stirred until homogeneous, then treated with acryloyl chloride (282 mg, 3.116 mmol) dropwise at rt. The resulting mixture was stirred for 3 h at 0° C., then quenched with water. The resulting mixture was extracted with DCM (50 mL×3). The combined organic layers were washed with brine, dried over anhydrous sodium sulfate, filtered and concentrated under vacuum. The residue was purified by Prep-HPLC (Column: XBridge Prep Phenyl OBD Column, 19*250 mm, 5 μm; Mobile Phase A: Water (10 mmol/L NH₄HCO₃), Mobile Phase B: ACN; Flow rate: 25 mL/min; Gradient: 50% B to 65% B in 9 min. 65% B; Wave Length: 254 nm; RT1(min): 8.23) to afford a racemic product, which was further separated by PREP-CHIRAL-HPLC with (Column: CHIRAL ART Amylose-C NEO, 2*25 cm. 5 μm; Mobile Phase A: Hex (0.5% 2M NH3-MeOH)—HPLC, Mobile Phase B: EtOH—HPLC; Flow rate: 20 mL/min; Gradient: 10% B to 10% B in 17 min; Wave Length: 220/254 nm; RT2(min): 15.256; Sample Solvent: EtOH—HPLC; Injection Volume: 0.4 mL) to give two products. The second eluting was lyophilized to afford (absolute stereochemistry unknown, randomly assigned as S) 1-[(3S*)-3-{2-[4-(trifluoromethyl)phenyl]ethynyl}pyrrolidin-1-yl]prop-2-en-1-one (75.8 mg, 12.36%) as a white solid. MS (ESI) calcd. for C₁₆H₁₄F₃NO, 293.00 m/z, found 294.1 0[M+H]⁺; ¹H NMR (400 MHz, DMSO-d₆) δ 7.77-7.70 (m, 4H), 7.66-7.59 (m, 4H), 6.67-6.55 (m, 2H), 6.22-6.11 (m, 2H), 5.75-5.64 (m, 2H), 4.01-3.52 (m, 6H), 3.50-3.33 (m, 5H), 2.38-2.18 (m, 2H), 2.14-1.90 (m, 2H); ¹⁹F NMR (376 MHz, DMSO-d6) δ −61.28.

### Example 68: Synthesis of (S, E)-2-fluoro-1-(3-(4-(trifluoromethyl)styryl)pyrrolidin-1-yl) prop-2-en-1-one

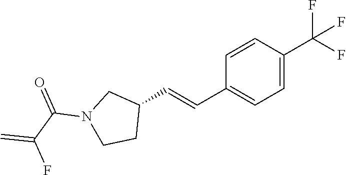 [SMILES: C=C(F)C(=O)N1CC[C@@H](/C=C/c2ccc(C(F)(F)F)cc2)C1]

### Synthetic Route:

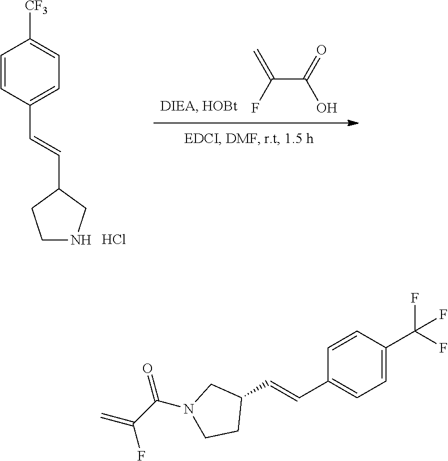

Synthesis of (S,E)-2-fluoro-1-(3-(4-(trifluoromethyl)styryl)pyrrolidin-1-yl)prop-2-en-1-one 2-Fluoroprop-2-enoic acid (421 mg, 4.68 mmol), EDCI (894 mg, 4.66 mmol) and HOBt (632 mg, 4.677 mmol) were added to DMF (10 ml) in an oven-dried 40 ml vial, and stirred until homogeneous, then (E)-3-(4-(trifluoromethyl)styryl)pyrrolidine hydrochloride (500 mg, 1.800 mmol) was added followed by DIEA (1.16 g, 8.99 mmol). The resulting mixture was stirred for 1.5 hours at room temperature, then concentrated under the reduced pressure. The residue was purified by reverse-phase chromatography (5%-60%, ACN/10 mM NH₄HCO₃ aqueous) to afford a racemic product, which was further separated by Prep-Chiral-HPLC (Column: CHIRAL ART Cellulose-SB, 2*25 cm, 5 μm; Mobile Phase A: Hex (0.5% 2M NH₃-MeOH)—HPLC. Mobile Phase B: IPA—HPLC; Flow rate: 20 mL/min; Gradient: 8% B to 8% B in 17 min; Wave Length: 220/254 nm; RT1(min): 13.115; RT2(min): 15.836; Sample Solvent: EtOH—HPLC; Injection Volume: 0.6 mL) to give two products. The first eluting fractions were lyophilized to afford (S, E)-2-fluoro-1-(3-(4-(trifluoromethyl)styryl)pyrrolidin-1-yl)prop-2-en-1-one as a white solid (64.4 mg, 11.4% yield). MS (ESI, m/z) calcd. for C₁₆H₁₅F₄NO, 313.10, found, 314.05 [M+H]⁺. ¹H NMR (400 MHz, DMSO-d₆) δ 7.71-7.60 (m, 4H), 6.68-6.58 (m, 1H), 6.57-6.45 (m, 1H), 5.50-5.24 (m, 2H), 3.91-3.73 (m, 1H), 3.71-3.52, 3.30-3.21 (m, 2H), 3.50-3.37 (m, 1H), 3.17-2.94 (m, 1H), 2.20-2.03 (m, 1H), 1.97-1.71 (m, 1H); ¹⁹F NMR (282 MHz, DMSO-d₆) δ: −60.82, 108.92.

### Example 69: Synthesis of (R,E)-2-fluoro-1-(3-(4-(trifluoromethyl)styryl)pyrrolidin-1-yl)prop-2-en-1-one

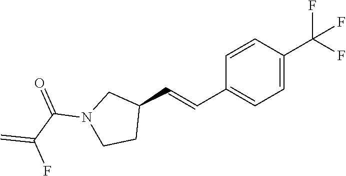 [SMILES: C=C(F)C(=O)N1CC[C@H](/C=C/c2ccc(C(F)(F)F)cc2)C1]

### Synthetic Route:

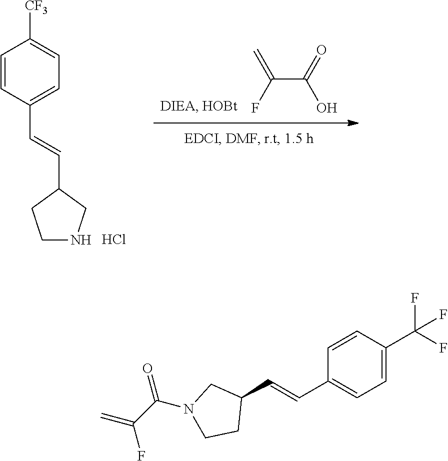

### Synthesis of (R, E)-2-fluoro-1-(3-(4-(trifluoromethyl)styryl)pyrrolidin-1-yl)prop-2-en-1-one

2-Fluoroprop-2-enoic acid (421 mg, 4.68 mmol), EDCI (894 mg, 4.66 mmol) and HOBt (632 mg, 4.68 mmol) were added to DMF (10 ml) in an oven-dried 40 ml vial, and stirred until homogeneous, then (E)-3-(4-(trifluoromethyl)styryl)pyrrolidine hydrochloride (500 mg, 1.80 mmol) was added followed by DIEA (1.16 g, 8.99 mmol). The resulting mixture was stirred for 1.5 hours at room temperature, then concentrated under the reduced pressure. The residue was purified by reverse-phase chromatography (5%-60%, ACN/10 mM NH₄HCO₃ aqueous solution) to afford a racemic product, which was further separated by Prep-Chiral-HPLC (Column: CHIRAL ART Cellulose-SB, 2*25 cm, 5 μm; Mobile Phase A: Hex (0.5% 2M NH₃-MeOH)—HPLC, Mobile Phase B: IPA—HPLC; Flow rate: 20 mL/min; Gradient: 8% B to 8% B in 17 min; Wave Length: 220/254 nm; RT1(min): 13.115; RT2(min): 15.836; Sample Solvent: EtOH—HPLC; Injection Volume: 0.6 mL) to give two products. The second eluting fraction was lyophilized to afford (R, E)-2-fluoro-1-(3-(4-(trifluoromethyl)styryl)pyrrolidin-1-yl)prop-2-en-1-one as a white solid (73.4 mg, 12.8% yield). MS (ESI, m/z) calcd. for C₁₆H₁₅F₄NO, 313.10, found, 314.00 [M+H]⁺. ¹H NMR (400 MHz, DMSO-d₆) δ 7.76-7.59 (m, 4H), 6.72-6.58 (m, 1H), 6.56-6.45 (m, 1H), 5.53-5.26 (m, 2H), 3.92-3.74 (m, 1H), 3.72-3.54, 3.30-3.23 (m, 2H), 3.49-3.39 (m, 1H), 3.14-2.95 (m, 1H), 2.21-1.99 (m, 1H), 1.92-1.70 (m, 1H); ¹⁹F NMR (282 MHz, DMSO-d₆) δ: −60.82, 108.92.

### Example 70: Synthesis of (E)-1-(3-(1H-pyrazol-1-yl)-4-(4-(trifluoromethyl)styryl) pyrrolidine-1-yl)prop-2-en-1-one

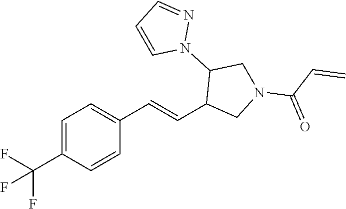 [SMILES: C=CC(=O)N1CC(/C=C/c2ccc(C(F)(F)F)cc2)C(n2cccn2)C1]

### Synthetic Route:

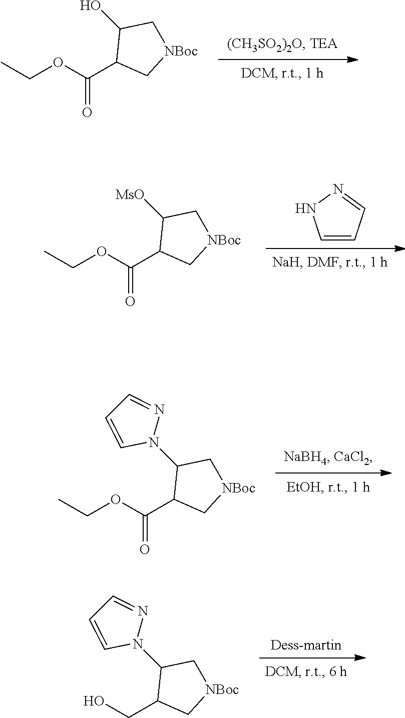

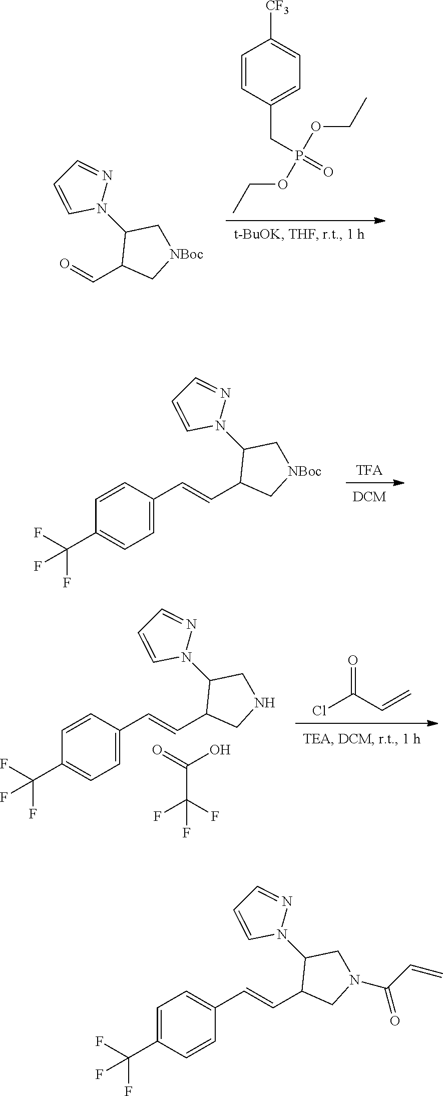

### Synthesis of 1-(tert-butyl) 3-ethyl 4-((methylsulfonyl)oxy)pyrrolidine-1,3-dicarboxylate

1-tert-Butyl 3-ethyl 4-hydroxypyrrolidine-1,3-dicarboxylate (3 g, 11.6 mmol), TEA (2.40 g, 23.7 mmol), a stir bar, and DCM (60 mL) were added to a 250 mL round-bottom flask and stirred until homogeneous, and then treated with methanesulfonic anhydride (3.0 g, 17.2 mmol). The resulting mixture was stirred for 1 hour at room temperature under a nitrogen atmosphere. The reaction mixture was then quenched with water (60 mL), extracted with DCM (3×120 mL). The combined organic layers were washed with brine, dried over anhydrous Na₂SO₄, filtered, and concentrated under the reduced pressure. The residue was purified by silica gel chromatography (40-60% EtOAc/PE) to afford 1-(tert-butyl) 3-ethyl 4-((methylsulfonyl)oxy)pyrrolidine-1,3-dicarboxylate as a light yellow oil (2.8 g, 71.7% yield). MS (ESI, m/z) calcd. for C₁₃H₂₃NO₇S, 337.12. found 281.95 [M+H-^{t}Bu]⁺.

### Synthesis of 1-(tert-butyl) 3-ethyl 4-(1H-pyrazol-1-yl)pyrrolidine-1,3-dicarboxylate

NaH (384 mg, 9.60 mmol, 60% in Oil), a stir bar and DMF (25 mL) were added to a 250 mL round-bottom flask and stirred until homogenous, then treated with pyrazole (654 mg, 9.60 mmol) at 0° C. under nitrogen atmosphere. After the solution was stirred for 15 min, 1-(tert-butyl) 3-ethyl 4-((methylsulfonyl) oxy)pyrrolidine-1,3-dicarboxylate (2.7 g, 8.00 mmol) in DMF (25 mL) was added dropwise at 0° C. The reaction mixture was warmed to room temperature and stirred for 1 hour, then quenched with water (50 mL), extracted with EA (3×100 mL). The combined organic layers were washed with brine, dried over anhydrous Na₂SO₄, filtered, and concentrated under the reduced pressure. The residue was purified by silica gel chromatography (20-40% EtOAc/PE) to afford 1-(tert-butyl) 3-ethyl 4-(1H-pyrazol-1-yl)pyrrolidine-1,3-dicarboxylate as a light yellow oil (0.9 g, 36.3% yield). MS (ESI, m/z) calcd. for C₁₅H₂₃N₃O₄, 309.17, found 254.05 [M+H-^{t}Bu]⁺.

### Synthesis of tert-butyl 3-(hydroxymethyl)-4-(1H-pyrazol-1-yl)pyrrolidine-1-carboxylate

1-(tert-Butyl) 3-ethyl 4-(1H-pyrazol-1-yl)pyrrolidine-1,3-dicarboxylate (850 mg, 2.75 mmol), NaBH₄ (125 mg, 3.30 mmol), a stir bar, and EtOH (16 mL) were added to a 50 mL round-bottom flask and stirred until homogeneous, and then treated with CaCl₂) (1.22 g, 11.0 mmol) at 0° C. The resulting mixture was stirred for 1 hour at room temperature under nitrogen atmosphere. The reaction mixture was then quenched with water (20 mL), and extracted with EA (3×40 mL). The combined organic layers were dried over anhydrous Na₂SO₄, filtered, and concentrated under the reduced pressure. The residue was purified by silica gel chromatography (40-60% EtOAc/PE) to afford tert-butyl 3-(hydroxymethyl)-4-(1H-pyrazol-1-yl) pyrrolidine-1-carboxylate as a light yellow oil (670 mg, 91.2% yield). MS (ESI, m/z) calcd. for C₁₃H₂₁N₃O₃, 267.16, found 212.15 [M+H-^{t}Bu]⁺.

### Synthesis of tert-butyl 3-formyl-4-(1H-pyrazol-1-yl)pyrrolidine-1-carboxylate

tert-butyl 3-(hydroxymethyl)-4-(aH-pyrazol-1-yl) pyrrolidine-1-carboxylate (570 mg, 2.13 mmol), a stir bar, and DCM (12 mL) were added to a 100 mL round-bottom flask and stirred until homogeneous, and then treated with Dess-martin (1.81 g, 4.27 mmol). The resulting mixture was stirred for 6 hours at room temperature under a nitrogen atmosphere. The reaction mixture was then quenched with water (10 mL), extracted with DCM (3×20 mL). The combined organic layers were washed with brine, dried over anhydrous Na₂SO₄, filtered, and concentrated under the reduced pressure to afford the crude product. The crude product was triturated with tert-butyl methyl ether and filtered. The filtrate was concentrated under vacuum to afford tert-butyl 3-formyl-4-(1H-pyrazol-1-yl)pyrrolidine-1-carboxylate as an off-white oil (490 mg, 86.6% yield). MS (ESI, m/z) calcd. for C₁₃H₁₉N₃O₃, 265.14, found 210.05 [M+H-^{t}Bu]⁺.

### Synthesis of tert-butyl (E)-3-(1H-pyrazol-1-yl)-4-(4-(trifluoromethyl)styryl)pyrrolidine-1-carboxylate

Diethyl (4-(trifluoromethyl)benzyl)phosphonate (340 mg, 1.15 mmol), tert-butyl 3-formyl-4-(1H-pyrazol-1-yl)pyrrolidine-1-carboxylate (305 mg, 1.15 mmol), a stir bar, and THF (6 mL) were added to a 50 mL round-bottom flask and stirred until homogeneous, and then treated with t-BuOK (386 mg, 3.44 mmol) at 0° C. The resulting mixture was stirred for 1 hour at room temperature. The reaction mixture was then quenched with water (6 mL), extracted with EA (3×10 mL). The combined organic layers were dried over anhydrous Na₂SO₄, filtered, and concentrated under the reduced pressure. The residue was purified by silica gel chromatography (20-30% EtOAc/PE) to afford tert-butyl (E)-3-(1H-pyrazol-1-yl)-4-(4-(trifluoromethyl)styryl)pyrrolidine-1-carboxylate as a dark yellow oil (40 mg, 8.6% yield). MS (ESI, m/z) calcd. for C₂₁H₂₄F₃N₃O₂, 407.18, found 352.05 [M+H-^{t}Bu]⁺.

### Synthesis of (E)-1-(4-(4-(trifluoromethyl)styryl)pyrrolidin-3-yl)-1H-pyrazole 2,2,2-trifluoroacetate

tert-Butyl (E)-3-(1H-pyrazol-1-yl)-4-(4-(trifluoromethyl)styryl)pyrrolidine-1-carboxylate (40 mg, 0.098 mmol), a stir bar, DCM (1 mL) were added to a 50 mL round-bottom flask and stirred until homogeneous, and then treated with TFA (0.3 mL). The reaction mixture was stirred for 2 hours at room temperature, then concentrated under the reduced pressure to afford (E)-1-(4-(4-(trifluoromethyl)styryl)pyrrolidin-3-yl)-1H-pyrazole 2,2,2-trifluoroacetate as a black solid (80 mg, crude). MS (ESI, m/z) calcd. for C₁₆H₁₆F₃N₃, 307.13, found, 308.10 [M+H]⁺.

### Synthesis of (E)-1-(3-(1H-pyrazol-1-yl)-4-(4-(trifluoromethyl)styryl)pyrrolidin-1-yl)prop-2-en-1-one

(E)-1-(4-(4-(Trifluoromethyl)styryl)pyrrolidin-3-yl)-1H-pyrazole 2,2,2-trifluoroacetate (80 mg, 0.190 mmol), TEA (96 mg, 0.949 mmol), a stir bar, and DCM (2 mL) were added to a 8 mL vial and stirred until homogeneous, and then treated with acryloyl chloride (34 mg, 0.376 mmol) at 0° C. The resulting mixture was stirred for 1 h at room temperature under nitrogen atmosphere. The reaction mixture was then quenched with water (2 mL), extracted with DCM (3×5 mL). The combined organic layers were washed with brine, dried over anhydrous Na₂SO₄, filtered, and concentrated under the reduced pressure. The residue was purified by reverse-phase chromatography (5%-50% ACN/10 mM NH₄HCO₃ aqueous solution) to afford (E)-1-(3-(1H-pyrazol-1-yl)-4-(4-(trifluoromethyl)styryl)pyrrolidin-1-yl)prop-2-en-1-one as a dark yellow oil (5.2 mg, 7.2% yield). MS (ESI, m/z) calcd. for C₁₉H₁₈F₃N₃O, 361.14, found 362.05 [M+H]⁺. ¹H NMR (400 MHz, DMSO-d₆) δ 7.89-7.40 (m, 4H), 7.35-6.90 (m, 4H), 6.71-6.53 (m, 1H), 6.50-6.41 (m, 1H), 6.30-6.12 (m, 1H), 5.87-5.65 (m, 1H), 5.07-4.88 (m, 1H), 4.28-3.84 (m, 2H), 3.84-3.41 (m, 3H); ¹⁹F NMR (376 MHz, DMSO-d₆) δ −60.89.

### Example 71: Synthesis of 1-(4-methoxy-4-((4-(trifluoromethyl)phenyl)ethynyl)piperidin-1-yl)prop-2-en-1-one

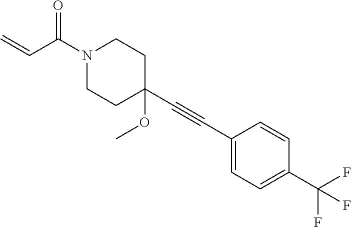 [SMILES: C=CC(=O)N1CCC(C#Cc2ccc(C(F)(F)F)cc2)(OC)CC1]

### Synthetic Route:

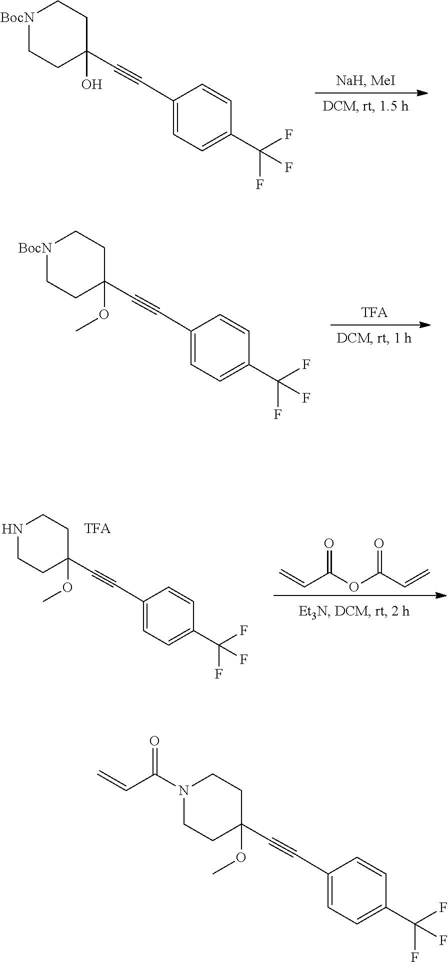

### Synthesis of tert-butyl 4-methoxy-4-((4-(trifluoromethyl)phenyl)ethynyl)piperidine-1-carboxylate

tert-Butyl 4-hydroxy-4-((4-(trifluoromethyl)phenyl)ethynyl)piperidine-1-carboxylate (100 mg, 0.271 mmol), CH₃I (369 mg, 2.60 mmol), a stir bar and DMF (8 mL) were added to 20 mL vial and stirred until homogenous, then treated 60% NaH (62 mg, 2.60 mmol) at room temperature. The resulting mixture was stirred for 1.5 hours at room temperature, then quenched with water. The resulting mixture was extracted with EA (30 mL) twice. The combined organic layers were washed with brine, dried over anhydrous sodium sulfate, filtered and concentrated under the reduced pressure. The residue was purified by silica gel chromatography (0-40% EtOAc/PE) to afford tert-butyl 4-methoxy-4-((4-(trifluoromethyl)phenyl)ethynyl) piperidine-1-carboxylate (608 mg, 73.2%) as a light yellow oil. MS (ESI, m/z) calcd. for C₂₀H₂₄F₃NO₃, 383.17, found, 328.05 [M+H-^{t}Bu]⁺.

Synthesis of 4-methoxy-4-((4-(trifluoromethyl)phenyl)ethynyl)piperidine 2,2,2-trifluoroacetate tert-Butyl 4-methoxy-4-((4-(trifluoromethyl)phenyl)ethynyl) piperidine-1-carboxylate (200 mg, 0.522 mmol). 2,2,2-trifluoroacetic acid (1 mL) a stir bar and DCM (4 mL) were added to 20 mL vial. The resulting mixture was stirred for 1 hour at room temperature, then concentrated under the reduced pressure to afford 4-methoxy-4-((4-(trifluoromethyl)phenyl)ethynyl)piperidine 2,2,2-trifluoroacetate (303 mg, crude) as a light yellow oil. MS (ESI, m/z) calcd. for C₁₅H₁₆F₃NO, 283.12, found, 284.10 [M+H]⁺.

### Synthesis of 1-(4-methoxy-4-((4-(trifluoromethyl)phenyl)ethynyl)piperidin-1-yl)prop-2-en-1-one

4-Methoxy-4-((4-(trifluoromethyl)phenyl)ethynyl)piperidine 2,2,2-trifluoroacetate (303 mg, 0.763 mmol), a stir bar, and DCM (2 ml) were added to a 8 ml vial and stirred until homogeneous before the reaction vessel was cooled to 0° C., then treated with Et₃N (231 mg, 2.28 mmol). The mixture was added a solution of acrylic anhydride (115 mg, 0.912 mmol) in DCM (2 ml) and stirred until homogeneous at 0° C., the resulting mixture was stirred at room temperature for 2 hours before quenched with water and extracted with DCM. The combined organic layers were washed with brine, dried over anhydrous sodium sulfate, filtered and concentrated under the reduced pressure. The residue was purified by Prep-HPLC (Column: XBridge Shield RP18 OBD Column, 30*150 mm, 5 μm; Mobile Phase A: Water (10 mmol/L NH₄HCO₃), Mobile Phase B: ACN; Flow rate: 60 mL/min; Gradient: 38% B to 68% B in 7 min, 68% B; Wave Length: 254 nm; RT1(min): 6.79; Injection Volume: 1.2 mL) to afford 1-(4-methoxy-4-((4-(trifluoromethyl) phenyl)ethynyl)piperidin-1-yl)prop-2-en-1-one as a colorless oil (80 mg, 31.1%). MS (ESI, m/z) mass calcd. for C₁₈H₁₈F₃NO₂, 337.12, found, 338.15 [M+H]⁺. ¹H NMR (400 MHz, DMSO-d₆) δ 7.81-7.69 (m, 4H), 6.83 (dd, J=16.7, 10.5 Hz, 1H), 6.11 (dd, J=16.7, 2.4 Hz, 1H), 5.69 (dd, J=10.5, 2.4 Hz, 1H), 3.92-3.72 (m, 2H), 3.62-3.41 (m, 2H), 3.38 (s, 3H), 1.98 (s, 2H), 1.85-1.69 (m, 2H).

### Example 72: Synthesis of 1-(4-fluoro-4-((4-(trifluoromethyl)phenyl)ethynyl)piperidin-1-yl)prop-2-en-1-one

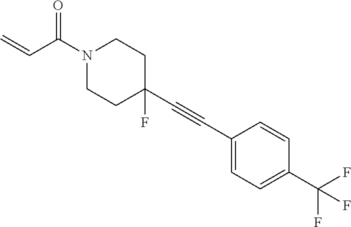 [SMILES: C=CC(=O)N1CCC(F)(C#Cc2ccc(C(F)(F)F)cc2)CC1]

### Synthetic Route

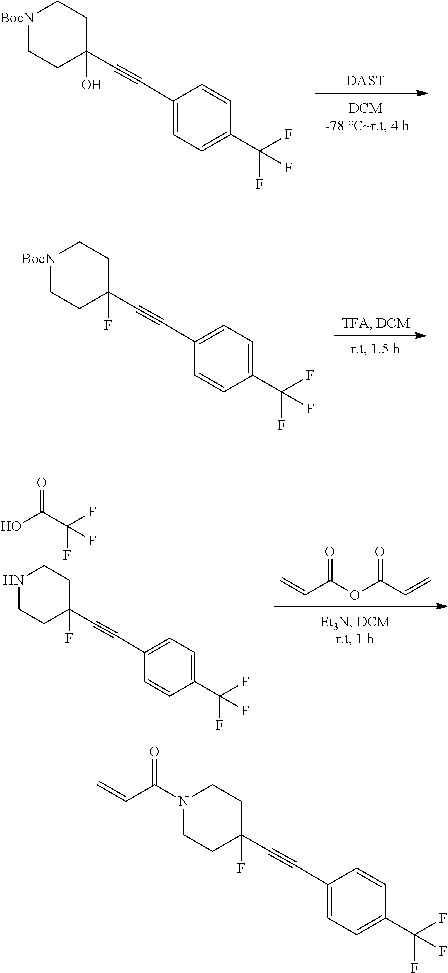

### Synthesis of tert-butyl 4-fluoro-4-((4-(trifluoromethyl)phenyl)ethynyl)piperidine-1-carboxylate

tert-Butyl 4-hydroxy-4-((4-(trifluoromethyl)phenyl)ethynyl)piperidine-1-carboxylate (600 mg, 1.62 mmol), a stir bar and DCM (8 mL) were added to a 100 mL flask and stirred until homogenous, then treated with a solution of diethyl(trifluoro-lambda4-sulfanyl)amine (393 mg, 2.44 mmol) in DCM (2 mL) at −78° C. The resulting mixture was stirred at −78° C. for 1 hour, then warmed to room temperature and stirred for 3 hours. The reaction was quenched with NH4Cl aqueous solution and extracted with DCM. The organic layers were combined, dried over Na2SO4, filtered and concentrated under the reduced pressure. The residue obtained was purified by silica gel chromatography (EA/PE=0-10%) to afford tert-butyl 4-fluoro-4-((4-(trifluoromethyl)phenyl) ethynyl)piperidine-1-carboxylate (430 mg, 71.3%) as a yellow solid. MS (ESI, m/z) calcd. for C₁₉H₂₁F₄NO₂: 371.15, found: 316.00 [M-^{t}Bu+H]⁺.

### Synthesis of 4-fluoro-4-((4-(trifluoromethyl)phenyl)ethynyl)piperidine 2,2,2-trifluoroacetate

tert-Butyl 4-fluoro-4-((4-(trifluoromethyl)phenyl)ethynyl)piperidine-1-carboxylate (410 mg, 1.10 mmol), a stir bar and DCM (15 mL) were added to a 100 mL flask and stirred until homogenous, then treated with TFA (3 mL) dropwise. The resulting mixture was stirred at room temperature for 1.5 hours. The reaction solution was concentrated under the reduced pressure to afford 4-fluoro-4-((4-(trifluoromethyl) phenyl)ethynyl)piperidine 2,2,2-trifluoroacetate (550 mg, crude). MS (ESI, m/z) calcd. for C₁₄H₁₃F₄N: 271.1, found: 272.00 [M+H]⁺.

### Synthesis of 1-(4-fluoro-4-((4-(trifluoromethyl)phenyl)ethynyl)piperidin-1-yl)prop-2-en-1-one

4-Fluoro-4-((4-(trifluoromethyl) phenyl)ethynyl)piperidine 2,2,2-trifluoroacetate (150 mg, 0.389 mmol), triethylamine (157 mg, 1.55 mmol), a stir bar and DCM (3 mL) were added to a 20 mL vial and stirred until homogenous, then treated with a solution of prop-2-enoyl prop-2-enoate (59 mg, 0.468 mmol) in DCM (1 mL). The resulting mixture was stirred at room temperature for 1 hour. The reaction was quenched with H₂O and extracted with DCM. The organic layers were combined, dried over anhydrous Na₂SO₄, filtered and concentrated under the reduced pressure. The residue obtained was purified by Prep-HPLC (Column: YMC-Actus Triart C18 ExRS, 20*250 mm, 5 μm; Mobile Phase A: Water (10 mmol/L NH4HCO3), Mobile Phase B: ACN; Flow rate: 25 mL/min; Gradient: 45% B to 70% B in 10 min, 70% B to 70% B in 12 min, 70% B; Wave Length: 254 nm; RT1(min): 11.13; Injection Volume: 0.5 mL) to afford 1-(4-fluoro-4-((4-(trifluoromethyl)phenyl)ethynyl)piperidin-1-yl)prop-2-en-1-one (25.0 mg, 19.5%) as a colorless oil. MS (ESI, m/z) calcd. for C₁₇H₁₅F₄NO, 325.11, found: 326.05 [M+H]¹. ¹H NMR (400 MHz, DMSO-d₆) δ 7.93-7.60 (m, 4H), 6.85 (dd, J=16.7, 10.5 Hz, 1H), 6.22-5.97 (m, 1H), 5.71 (dd, J=10.5, 2.4 Hz, 1H), 3.90-3.53 (m, 4H), 2.24-1.93 (m, 4H); ¹⁹F NMR (376 MHz, DMSO-d₆) δ: −61.31.

### Example 73: Synthesis of 1-(4-hydroxy-4-((4-(trifluoromethyl)phenyl)ethynyl)piperidin-1-yl)prop-2-en-1-one

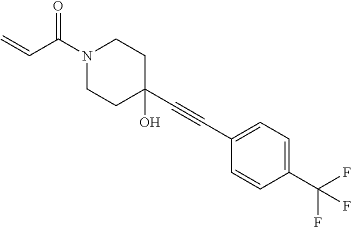 [SMILES: C=CC(=O)N1CCC(O)(C#Cc2ccc(C(F)(F)F)cc2)CC1]

### Synthetic Route:

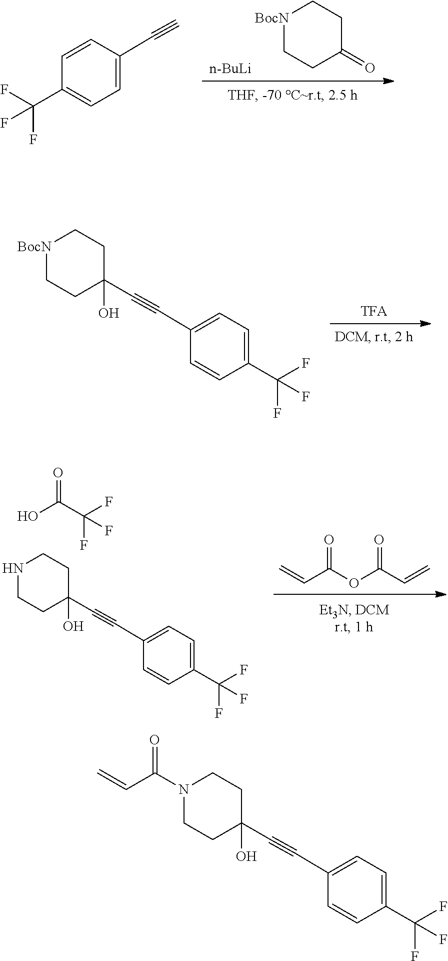

### Synthesis of tert-butyl 4-hydroxy-4-((4-(trifluoromethyl)phenyl)ethynyl)piperidine-1-carboxylate

1-Ethynyl-4-(trifluoromethyl)benzene (1.88 g, 11.0 mmol), a stir bar, and THF (30 mL) were added to a 3-necked round-bottom flask, and stirred until homogenous, then treated with butyllithium (5.2 mL, 13.0 mmol) dropwise at −78° C. under nitrogen. After the solution was stirred for 30 min, a solution of tert-butyl 4-oxopiperidine-1-carboxylate (2.0 g, 10.0 mmol) in THF (10 mL) was added at −78° C. The resulting mixture was stirred at room temperature for 2 hours. The reaction was quenched with NH₄Cl aqueous solution and extracted with EA. The organic layers were combined, dried over anhydrous Na₂SO₄, filtered and concentrated under the reduced pressure. The residue obtained was purified by silica gel chromatography (EA/PE=0-30%) to afford tert-butyl 4-hydroxy-4-((4-(trifluoromethyl)phenyl)ethynyl)piperidine-1-carboxylate (3.1 g, 83.6%) as a yellow oil. MS (ESI, m/z) calcd. for C₁₉H₂₂F₃NO₃: 369.16. found: 314.10 [M-^{t}Bu+H]¹.

### Synthesis of 4-((4-(trifluoromethyl)phenyl)ethynyl)piperidin-4-ol 2,2,2-trifluoroacetate

tert-Butyl 4-hydroxy-4-((4-(trifluoromethyl)phenyl)ethynyl)piperidine-1-carboxylate (300 mg, 0.812 mmol), a stir bar and DCM (10 mL) were added to a 100 mL flask and stirred until homogenous, then treated with TFA (2 mL). The resulting mixture was stirred at room temperature for 2 hours. The reaction solution was concentrated under the reduced pressure to afford 4-((4-(trifluoromethyl) phenyl)ethynyl)piperidin-4-ol 2,2,2-trifluoroacetate (300 mg, 96.4%) as a yellow oil. MS (ESI, m/z) calcd. for C₁₄H₁₄F₃NO: 269.10, found: 270.05 [M+H]⁺.

### Synthesis of 1-(4-hydroxy-4-((4-(trifluoromethyl)phenyl)ethynyl)piperidin-1-yl)prop-2-en-1-one

4-((4-(Trifluoromethyl)phenyl)ethynyl)piperidin-4-ol 2,2,2-trifluoroacetate (150 mg, 0.391 mmol), triethylamine (158 mg, 1.56 mmol), a stir bar and DCM (3 mL) were added to a 20 mL vial and stirred until homogenous, then treated with a solution of prop-2-enoyl prop-2-enoate (59 mg, 0.468 mmol) in DCM (1 mL). The resulting mixture was stirred at room temperature for 1 hour. The reaction was quenched with H₂O and extracted with DCM. The organic layers were combined, dried over Na₂SO₄, filtered and concentrated under the reduced pressure. The residue obtained was purified by Prep-HPLC (Column: XBridge Shield RP18 OBD Column, 30*150 mm, 5 μm; Mobile Phase A: Water (10 mmol/L NH₄HCO₃), Mobile Phase B: ACN; Flow rate: 60 mL/min; Gradient: 29% B to 59% B in 7 min, 59% B: Wave Length: 254 nm; RT1(min): 5.27; Injection Volume: 1.4 mL) to afford 1-(4-hydroxy-4-((4-(trifluoromethyl)phenyl)ethynyl) piperidin-1-yl)prop-2-en-1-one (42.4 mg, 33.4%) as a white solid. MS (ESI, m/z) calcd. for C₁₇H₁₆F₃NO₂, 323.11, found: 324.15 [M+H]⁺. ¹H NMR (400 MHz, DMSO-d₆) δ 7.79-7.71 (m, 2H), 7.71-7.64 (m, 2H), 6.83 (dd, J=16.7, 10.5 Hz, 1H), 6.10 (dd, J=16.7, 2.4 Hz, 1H), 5.91 (s, 1H), 5.68 (dd, J=10.5, 2.4 Hz, 1H), 3.90-3.70 (m, 2H), 3.60-3.38 (m, 2H), 1.98-1.81 (m, 2H), 1.80-1.60 (m, 2H); ¹⁹F NMR (376 MHz, DMSO-d₆) δ: −61.30.

### Example 74: Synthesis of 1-(4-methyl-4-((4-(trifluoromethyl)phenyl)ethynyl)piperidin-1-yl)prop-2-en-1-one

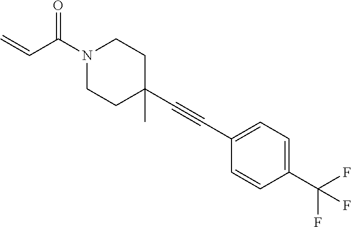 [SMILES: C=CC(=O)N1CCC(C)(C#Cc2ccc(C(F)(F)F)cc2)CC1]

### Synthetic Route:

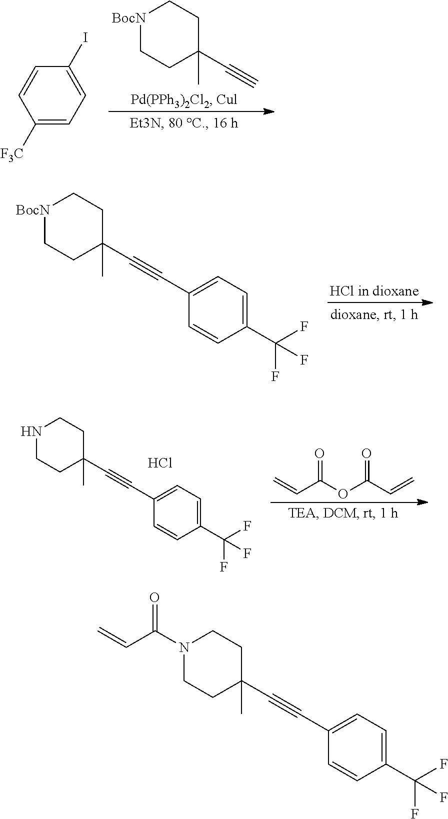

### Synthesis of tert-butyl 4-methyl-4-((4-(trifluoromethyl)phenyl)ethynyl)piperidine-1-carboxylate

1-Iodo-4-(trifluoromethyl)benzene (300 mg, 1.10 mmol), CuI (42 mg, 0.221 mmol), Pd(PPh₃)₂Cl₂ (155 mg, 0.221 mmol), a stir bar, TEA (10 mL) were added to a 40 mL vial and stirred until homogeneous, then treated with tert-butyl 4-ethynyl-4-methylpiperidine-1-carboxylate (247 mg, 1.11 mmol). The mixture was stirred at 80° C. for 16 hours, then cooled to room temperature and filtered. The filtrate was diluted with H₂O (30 mL) solution and extracted with EA (30 mL×3). The combined organic layers were washed with brine. The combined extracts were dried over anhydrous sodium sulfate, filtered, and concentrated under the reduced pressure. The residue was subjected to silica gel chromatography (0-30% EA/PE) to afford tert-butyl 4-methyl-4-((4-(trifluoromethyl)phenyl)ethynyl)piperidine-1-carboxylate (360 mg, 88.8% yield) as a yellow oil. MS (ESL m/z) calcd. for C₂₀H₂₄F₃NO₂, 367.17, found, 353.05 [M-56+ACN+H]⁺.

### Synthesis of 4-methyl-4-((4-(trifluoromethyl)phenyl)ethynyl)piperidine hydrochloride

tert-Butyl 4-methyl-4-(2-[4-(trifluoromethyl)phenyl]ethynylpiperidine-1-carboxylate (170 mg, 0.463 mmol), a stir bar., 1,4-diaxane (1 mL) were added to a 50 mL round-bottom flask and stirred until homogeneous, then treated with HCl in 1,4-dioxane (2 mL). The mixture was stirred for 1 hour at room temperature, then concentrated under the reduced pressure to afford 4-methyl-4-((4-(trifluoromethyl)phenyl)ethynyl)piperidine hydrochloride (150 mg, crude) as a white solid. MS (ESI, m/z) calcd. for C₁₅H₁₆F₃N, 267.12, found, 268.05 [M+H]⁺.

### Synthesis of 1-(4-methyl-4-((4-(trifluoromethyl)phenyl)ethynyl)piperidin-1-yl)prop-2-en-1-one

4-Methyl-4-((4-(trifluoromethyl)phenyl)ethynyl)piperidine hydrochloride (150 mg, 0.494 mmol), a stir bar. Et₃N (250 mg, 2.471 mmol), DCM (3 mL) were added to a 20 mL vial and stirred until homogeneous at 0° C., then treated with a solution of prop-2-enoyl prop-2-enoate (187 mg, 1.483 mmol) in DCM (1 mL). The resulting mixture was stirred at room temperature for 1 h, then diluted with water (50 mL) and extracted with DCM (50 mL×3). The combined extracts were dried over anhydrous sodium sulfate, filtered, and concentrated under the reduced pressure. The residue was purified by HPLC (Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 42% B to 72% B in 7 min, 72% B; Wave Length: 254 nm; RT1(min): 6.62; Injection Volume: 1.6 mL) to afford 1-(4-methyl-4-(2-[4-(trifluoromethyl)phenyl]ethynylpiperidin-1-yl)prop-2-en-1-one (69.4 mg, 43.7% yield) as a colorless oil. MS (ESI, m/z) calcd. for C₁₈H₁₈F₃NO, 321.34, found, 322.05 [M+H]⁺. ¹H NMR (400 MHz, DMSO-d₆) δ 7.92-7.43 (m, 4H), 6.83 (dd, J=16.7, 10.5 Hz, 1H), 6.10 (dd, J=16.8, 2.4 Hz, 1H), 5.68 (dd, J=10.5, 2.4 Hz, 1H), 4.39 (d, J=13.3 Hz, 1H), 4.03 (d, J=13.8 Hz, 1H), 2.98 (t, J=12.6 Hz, 1H), 3.40-3.35 (m, 1H), 1.79 (d, J=13.1 Hz, 2H), 1.56-1.38 (m, 2H), 1.33 (s, 3H); ¹⁹F NMR (376 MHz, DMSO-d₆) δ: −61.2354.

### Example 75: Synthesis of 1-(3-((3-(trifluoromethyl)phenyl)ethynyl)piperidin-1-yl)prop-2-en-1-one

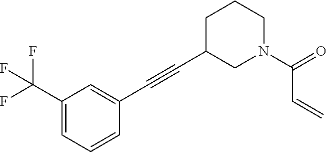 [SMILES: C=CC(=O)N1CCCC(C#Cc2cccc(C(F)(F)F)c2)C1]

### Synthetic Route:

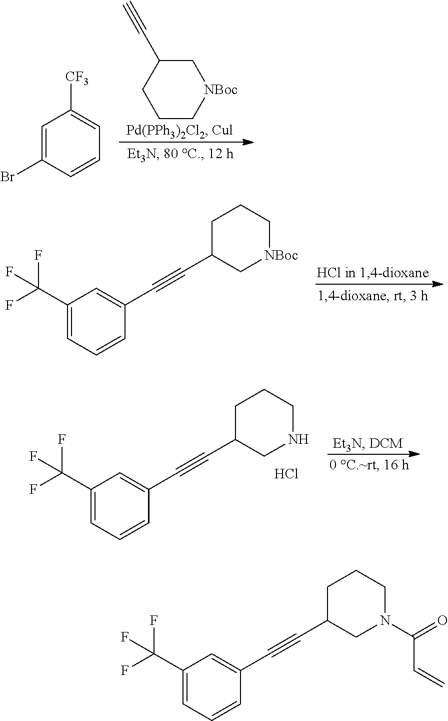 [SMILES: FC(F)(F)c1cccc(Br)c1 ; C#CC1CCCN(C(=O)OC(C)(C)C)C1 ; CC(C)(C)OC(=O)N1CCCC(C#Cc2cccc(C(F)(F)F)c2)C1 ; FC(F)(F)c1cccc(C#CC2CCCNC2)c1 ; Cl ; C=CC(=O)N1CCCC(C#Cc2cccc(C(F)(F)F)c2)C1]

### Synthesis of tert-butyl 3-((3-(trifluoromethyl)phenyl)ethynyl)piperidine-1-carboxylate

1-Bromo-3-(trifluoromethyl)benzene (500 mg, 2.22 mmol), a stir bar, TEA (5 mL) and tert-butyl 3-ethynylpiperidine-1-carboxylate (466 mg, 2.23 mmol) were added to an oven-dried and nitrogen-purged 25 mL round-bottom flask and stirred until homogenous, then treated with Pd(PPh₃)₂Cl₂ (313 mg, 0.446 mmol) and CuI (85 mg, 0.446 mmol) in batches at room temperature. The resulting mixture was stirred for 12 h at 80° C. under nitrogen atmosphere, then cooled to room temperature. The reaction solution was quenched with water, and extracted with DCM (10 mL×3). The combined organic layers were washed with brine, dried over anhydrous sodium sulfate, filtered and concentrated under vacuum. The residue was purified by silica gel chromatography (0%-40% EA in PE) to afford tert-butyl 3-((3-(trifluoromethyl) phenyl)ethynyl)piperidine-1-carboxylate (500 mg, 88.8%) as a yellow oil. MS (ESI, m/z) mass calcd. for C₁₄H₁₄F₃N, 353.16, found, 254.05 [M-56+H]⁺.

### Synthesis of 3-((3-(trifluoromethyl)phenyl)ethynyl)piperidine hydrochloride

tert-Butyl 3-((3-(trifluoromethyl)phenyl)ethynyl)piperidine-1-carboxylate (300 mg, 0.847 mmol), a stir bar and 1,4-dioxane (2 mL) were added to a 25 mL round bottom flask and stirred until homogeneous, then treated with 4M HCl in 1,4-dioxane (2 mL) dropwise at 0° C. The resulting mixture was stirred for 3 h at room temperature, concentrated under the reduced pressure to afford 3-((3-(trifluoromethyl)phenyl)ethynyl) piperidine hydrochloride (290 mg, crude) as a yellow solid. MS (ESI, m/z) calcd. for C₁₄H₁₄F₃N, 253.11, found, 254.05 [M+H]⁺.

### Synthesis of 1-(3-((3-(trifluoromethyl)phenyl)ethynyl)piperidin-1-yl)prop-2-en-1-one

3-((3-(Trifluoromethyl)phenyl)ethynyl) piperidine hydrochloride (290 mg, 1.00 mmol), a stir bar, Et₃N (347 mg, 3.43 mmol) and DCM (4.5 mL) were added to 25 mL round bottom flask and stirred until homogeneous, then treated with a solution of prop-2-enoyl prop-2-enoate (216 mg, 1.71 mmol) in DCM (0.5 mL) dropwise at 0° C. The resulting mixture was stirred overnight at room temperature, diluted with water (5 mL) and extracted with DCM (5 mL×3). The combined organic layers were washed with brine, dried over anhydrous sulfate sodium, filtered and concentrated under the reduced pressure. The residue was by reverse-phase chromatography (5%-70% ACN/10 mM NH₄HCO₃ aqueous solution) to afford 1-(3-((3-(trifluoromethyl)phenyl) ethynyl)piperidin-1-yl)prop-2-en-1-one as a colorless oil (79.9 mg, 25.5%). MS (ESI, m/z) mass calcd. for C₁₇H₁₆F₃NO, 307.12, found, 308.05 [M+H]⁺. ¹H NMR (400 MHz, DMSO-d₆) δ 7.78-7.68 (m, 3H), 7.64-7.56 (m, 1H), 6.82 (dd, J=16.6, 10.5 Hz, 1H), 6.09 (dd, J=16.7, 2.5 Hz, 1H), 5.67 (dd, J=10.5, 2.5 Hz, 1H), 3.94-3.79 (m, 2H), 3.47-3.39 (m, 1H), 3.31-3.27 (m, 1H), 3.04-2.94 (m, 1H), 1.90-1.85 (m, 2H), 1.60-1.55 (m, 2H); ¹⁹F NMR (376 MHz, DMSO-d₆) δ: −61.39.

### Example 76: Synthesis of 1-(3-((4-(trifluoromethyl)phenyl)ethynyl)piperidin-1-yl)prop-2-en-1-one

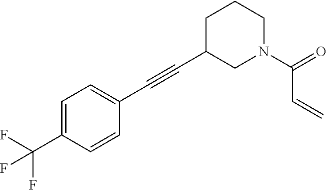 [SMILES: C=CC(=O)N1CCCC(C#Cc2ccc(C(F)(F)F)cc2)C1]

### Synthetic Route:

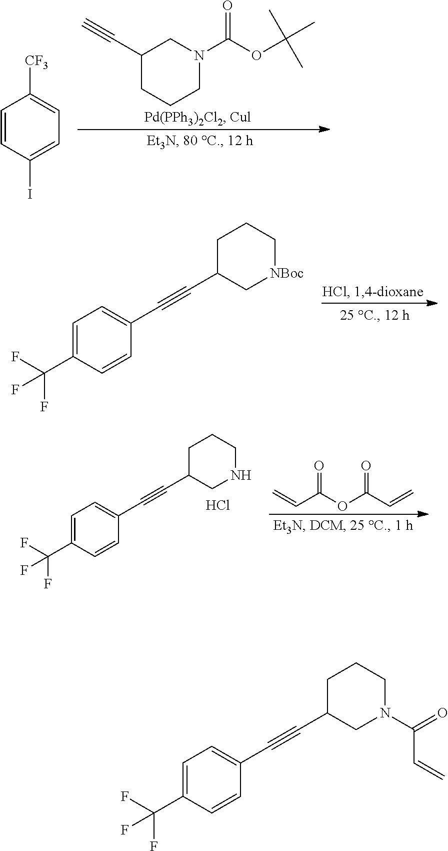

### Synthesis of tert-butyl 3-((4-(trifluoromethyl)phenyl)ethynyl)piperidine-1-carboxylate

1-Iodo-4-(trifluoromethyl)benzene (500 mg, 1.84 mmol), a stir bar, TEA (5 mL) and tert-butyl 3-ethynylpiperidine-1-carboxylate (384 mg, 1.84 mmol) were added to an oven-dried and nitrogen-purged 25 mL round-bottom flask and stirred until homogenous, then treated with Pd(PPh₃)₂Cl₂ (258 mg, 0.368 mmol,) and CuI (70 mg, 0.368 mmol) in batches at rt. The resulting mixture was stirred for 12 hours at 80° C. under nitrogen atmosphere and cooled to room temperature. The reaction was then quenched with water, and extracted with DCM (10 mL×3). The combined organic layers were washed with brine, dried over anhydrous Na₂SO₄, filtered and concentrated under the reduced pressure. The residue was purified by silica gel chromatography (0% to 40% EA in PE) to afford tert-butyl 3-((4-(trifluoromethyl)phenyl]ethynylpiperidine-1-carboxylate (500 mg, 77.0%) as a yellow oil. MS (ESI, m/z) calcd. for C₁₉H₂₂F₃NO₂, 353.00, found 254.00[M-Boc+H]⁺.

### Synthesis of 3-((4-(trifluoromethyl)phenyl)ethynyl)piperidine hydrochloride

tert-Butyl 3-((4-(trifluoromethyl)phenyl]ethynylpiperidine-1-carboxylate (200 mg. 0.566 mmol), a stir bar and dioxane (0.5 mL) were added to a 8 mL vial and stirred until homogenous, then treated with HCl in dioxane (1.5 mL, 49.4 mmol) dropwise at room temperature. The resulting mixture was stirred for 12 hours at room temperature, then concentrated under the reduced pressure to afford 3-((4-(trifluoromethyl)phenyl)ethynyl)piperidine hydrochloride (220 mg, crude) as a white solid. MS (ESI, m/z) calcd for C₁₄H₁₄F₃N, 253.00, found, 254.05[M+H]⁺.

### Synthesis of 1-(3-((4-(trifluoromethyl)phenyl)ethynyl)piperidin-1-yl)prop-2-en-1-one

3-((4-(Trifluoromethyl)phenyl)ethynyl)piperidine hydrochloride (220 mg, 0.759 mmol), a stir bar, DCM (3 mL) and TEA (347 mg, 3.43 mmol) were added to a 8 mL vial and stirred until homogenous, then treated with a solution of prop-2-enoyl prop-2-enoate (164 mg, 1.30 mmol) in DCM dropwise at room temperature. The resulting mixture was stirred for 1 h, then quenched with water. The resulting mixture was extracted with DCM (5 mL×3). The combined organic layers were washed with brine, dried over anhydrous sodium sulfate, filtered and concentrated under vacuum. The residue was further purified by reverse-phase chromatography (5%-50% ACN/0.1% FA water) to afford 1-(3-((4-(trifluoromethyl)phenyl)ethynyl piperidin-1-yl)prop-2-en-1-one (134 mg, 57.5%) as a colorless oil. MS (ESI, m/z) calcd. for C₁₇H₁₆F₃NO, 307.00, found, 308.05 [M+H]⁺. ¹H NMR (400 MHz, DMSO-d₆) δ 7.76-7.69 (m, 2H), 7.66-7.59 (m, 2H), 6.82 (dd, J=16.7, 10.5 Hz, 1H), 6.10 (dd, J=16.7, 2.4 Hz, 1H), 5.68 (dd, J=10.5, 2.5 Hz, 1H), 3.89 (dd, J=34.2, 13.1 Hz, 2H), 3.47-3.36 (m, 1H), 3.33-3.23 (m, 1H), 3.08-2.94 (m, 1H), 1.89 (s, 2H), 1.59 (s, 2H); ¹⁹F NMR (376 MHz, DMSO-d₆) δ: −61.24.

### Example 77: Synthesis of (E)-1-(3-(4-(trifluoromethyl)styryl)azetidin-1-yl)but-2-yn-1-one

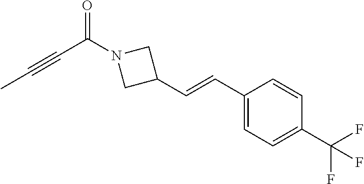 [SMILES: CC#CC(=O)N1CC(/C=C/c2ccc(C(F)(F)F)cc2)C1]

### Synthetic Route:

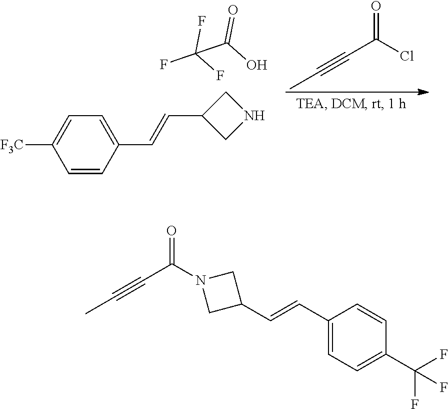

### Synthesis of (E)-1-(3-(4-(trifluoromethyl)styryl)azetidin-1-yl)but-2-yn-1-one

(E)-3-(4-(Trifluoromethyl)styryl)azetidine 2,2,2-trifluoroacetate (280 mg, 0.821 mmol), DCM (4.5 mL), TEA (500 mg, 4.94 mmol) and a stir bar were added to a 20 mL vial and stirred until homogenous, then treated with a solution of but-2-ynoyl chloride (101 mg, 0.985 mmol) in DCM (0.5 mL) at 0° C. The resulting mixture was stirred for 1 h at room temperature, then diluted with water (50 mL) and extracted with DCM (50 mL×2). The combined organic layers were washed with brine, dried over anhydrous sodium sulfate, filtered and concentrated under the reduced pressure. The resulting residue was purified by Prep-HPLC with (Column: XSelect CSH Prep C18 OBD Column, 19*150 mm, 5 μm; Mobile Phase A: Water (0.1% FA), Mobile Phase B: ACN; Flow rate: 25 mL/min; Gradient: 37% B to 67% B in 7 min, 67% B; Wave Length: 254 nm; RT1(min): 5.87; Injection Volume: 0.8 mL) to afford (E)-1-(3-(4-(trifluoromethyl)styryl)azetidin-1-yl)but-2-yn-1-one as a light yellow oil (32.4 mg, 13.4% yield). MS (ESI, m/z) calcd. for C₁₆H₁₄F₃NO, 293.10, found, 294.05 [M+H]⁺. ¹H NMR (400 MHz, DMSO-d₆) δ 7.73-7.63 (m, 4H), 6.81-6.70 (m, 1H), 6.67-6.58 (m, 1H), 4.42-4.33 (m, 1H), 4.19-4.10 (m, 1H), 4.09-4.01 (m, 1H), 3.85-3.77 (m, 1H), 3.59-3.47 (m, 1H), 2.01 (s, 3H); ¹⁹F NMR (376 MHz, DMSO-d₆) δ −60.85.

### Example 78: Synthesis of (E)-1-(3-(4-(trifluoromethyl)styryl)azetidin-1-yl)prop-2-yn-1-one

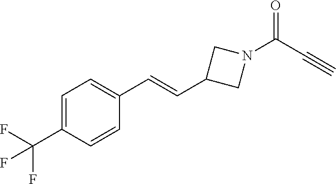 [SMILES: C#CC(=O)N1CC(/C=C/c2ccc(C(F)(F)F)cc2)C1]

### Synthetic Route:

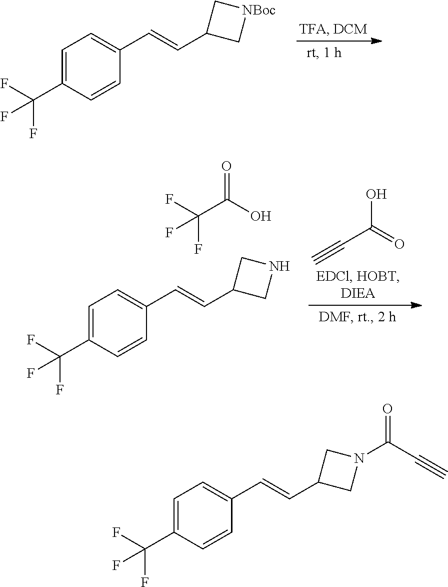

### Synthesis of (E)-3-(4-(trifluoromethyl)styryl)azetidine 2,2,2-trifluoroacetate

tert-Butyl 3-((E)-2-(4-(trifluoromethyl)phenyl]ethenyl)azetidine-1-carboxylate (200 mg, 0.611 mmol), 2,2,2-trifluoroacetic acid (1 mL), a stir bar and DCM (4 mL) were added to 20 mL vial. The resulting mixture was stirred for 1 hour at room temperature, then concentrated under vacuum to afford (E)-3-(4-(trifluoromethyl)styryl)azetidine 2,2,2-trifluoroacetate (354 mg, crude) as a brown oil. MS (ESI, m/z) calcd. for C₄H₁₃F₆NO₂, 277.09, found, 228.00 [M+H]⁺.

### Synthesis of (E)-1-(3-(4-(trifluoromethyl)styryl)azetidin-1-yl)prop-2-yn-1-one

Propiolic acid (108 mg, 1.55 mmol), DIEA (667 mg, 5.16 mmol), EDCI (297 mg, 1.55 mmol), HOBT (209 mg, 1.55 mmol), a stir bar and DMF (4 mL) were added to 40 mL vial and stirred until homogenous, then treated with (E)-3-(4-(trifluoromethyl)styryl)azetidine 2,2,2-trifluoroacetate (352 mg, 1.03 mmol) in DMF (2 mL) at room temperature. The resulting mixture was stirred for 2 hours at room temperature, then concentrated under the reduced pressure. The residue was purified by reverse-phase chromatography (5%-50% ACN/10 mM NH₄HCO₃ aqueous solution) to afford a crude product, which was further purified by Prep-HPLC with (Column: Xselect CSH C18 OBD Column 30*150 mm 5 m, n; Mobile Phase A: Water (0.1% FA), Mobile Phase B: ACN; Flow rate: 60 mL/min; Gradient: 35% B to 65% B in 7 min, 65% B; Wave Length: 254 nm; RT1(min): 5.25; Injection Volume: 0.54 mL) to afford (E)-1-(3-(4-(trifluoromethyl)styryl)azetidin-1-yl)prop-2-yn-1-one as a white solid (7.2 mg, 2.4%). MS (ESI, m/z) calcd. for C₁₅H₁₂F₃NO, 279.09, found, 280.00 [M+H]⁺. 1H NMR (400 MHz, DMSO-d₆) δ 7.80-7.52 (m, 4H), 6.82-6.71 (m, 1H), 6.67-6.59 (m, 1H), 4.47 (s, 1H), 4.45-4.36 (m, 1H), 4.22-4.13 (m, 1H), 4.09 (dd, J=8.9, 6.0 Hz, 1H), 3.84 (dd, J=10.1, 6.1 Hz, 1H), 3.60-3.48 (m, 1H).

### Example 79: Synthesis of 1-(4-((3-(trifluoromethyl)phenyl)ethynyl)piperidin-1-yl)prop-2-en-1-one

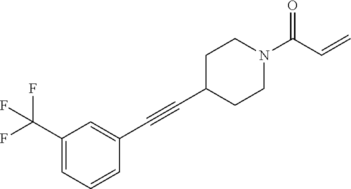 [SMILES: C=CC(=O)N1CCC(C#Cc2cccc(C(F)(F)F)c2)CC1]

### Synthetic Route:

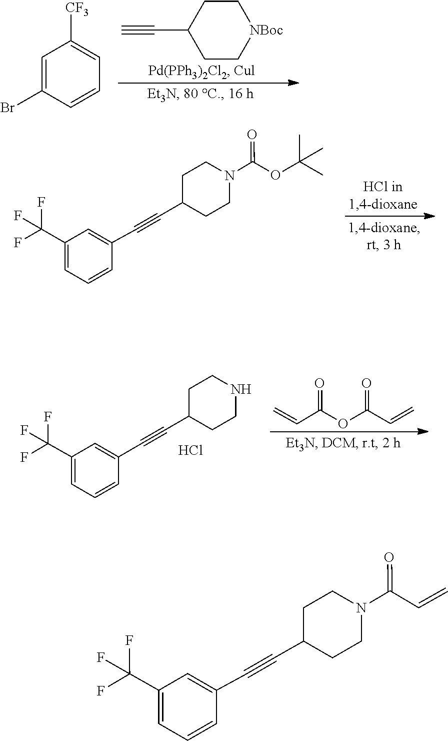

### Synthesis of tert-butyl 4-((3-(trifluoromethyl)phenyl)ethynyl)piperidine-1-carboxylate

1-Bromo-3-(trifluoromethyl)benzene (500 mg, 2.22 mmol), a stir bar, tert-butyl 4-ethynylpiperidine-1-carboxylate (560 mg, 2.68 mmol), Pd(PPh₃)₂Cl₂ (312 mg, 0.445 mmol), CuI (84 mg, 0.441 mmol) and Et₃N (10 mL) were added to a 40 mL sealed vial and stirred until homogeneous. The resulting mixture was stirred overnight at 80° C. under nitrogen atmosphere, then cooled to room temperature, concentrated under the reduced pressure. The residue was purified by reverse chromatography with ACN/H₂O (0.05% TFA) to give tert-butyl 4-((3-(trifluoromethyl)phenyl)ethynyl)piperidine-1-carboxylate (320 mg, 40.8%) as a white solid. MS (ESI, m/z) calcd. for C₁₉H₂₂F₃NO₂, 353.16, found, 298.00 [M+H-^{t}Bu]⁺.

### Synthesis of 4-((3-(trifluoromethyl)phenyl)ethynyl)piperidine hydrochloride

tert-Butyl 4-((3-(trifluoromethyl)phenyl)ethynyl)piperidine-1-carboxylate (300 mg, 0.849 mmol), a stir bar and 1,4-dioxane (2 mL) were added to a 25 mL round bottom flask and stirred until homogeneous, then treated with HCl in 1,4-dioxane (2 mL, 4M, 8 mmol) dropwise at 0° C. The resulting mixture was stirred for 3 h at room temperature, concentrated under the reduced pressure to give 4-((3-(trifluoromethyl)phenyl)ethynyl) piperidine hydrochloride (260 mg, crude) as a white solid. MS (ESI, m/z) calcd. for C₁₄H₁₄F₃N, 253.11, found, 254.05 [M+H]¹.

### Synthesis of 1-(4-((3-(trifluoromethyl)phenyl)ethynyl)piperidin-1-yl)prop-2-en-1-one

4-((3-(Trifluoromethyl)phenyl)ethynyl)piperidine hydrochloride (120 mg, 0.414 mmol), a stir bar, Et₃N (143 mg, 1.41 mmol) and DCM (1.5 mL) were added to a 25 mL round bottom flask and stirred until homogeneous, then treated with a solution of prop-2-enoyl prop-2-enoate (90 mg, 0.714 mmol) in DCM (0.5 mL) dropwise at 0° C. The resulting mixture was stirred for 2 h at room temperature, diluted with water (5 mL) and extracted with DCM (5 mL×3). The combined organic layers were washed with brine, dried over anhydrous sulfate sodium, filtered and concentrated under the reduced pressure. The residue was purified by PREP-HPLC (Column: XBridge Prep OBD C18 Column. 19*250 mm, 5 μm; Mobile Phase A: Water (10 mmol/L NH4HCO3), Mobile Phase B: ACN; Flow rate: 25 mL/min; Gradient: 55% B to 85% B in 7 min, 85% B; Wave Length: 254 nm; RT1(min): 5.25; Injection Volume: 2 mL) to give 1-(4-((3-(trifluoromethyl)phenyl) ethynyl)piperidin-1-yl)prop-2-en-1-one as a colorless oil (39.8 mg, 31.2%). MS (ESI, m/z) mass calcd. for C₁₇H₁₆F₃NO, 307.12, found, 308.05 [M+H]⁺. ¹H NMR (400 MHz, DMSO-d₆) δ 7.78-7.69 (m, 3H), 7.65-7.56 (m, 1H), 6.82 (dd, J=16.7, 10.5 Hz, 1H), 6.10 (dd, J=16.7, 2.4 Hz, 1H), 5.68 (dd, J=10.5, 2.4 Hz, 1H), 3.93-3.79 (m, 2H), 3.44 (d, J=11.0 Hz, 1H), 3.32-3.25 (m, 1H), 3.06-2.95 (m, 1H), 1.94-1.84 (m, 2H), 1.65-1.55 (m, 2H); ¹⁹F NMR (376 MHz, DMSO-d₆) δ: −61.39.

### Example 80: Synthesis of 1-(4-((4-(trifluoromethyl)phenyl)ethynyl)piperidin-1-yl)prop-2-en-1-one

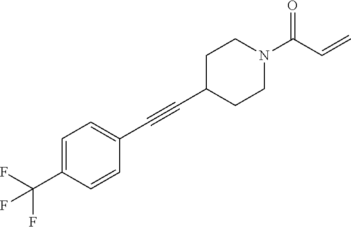 [SMILES: C=CC(=O)N1CCC(C#Cc2ccc(C(F)(F)F)cc2)CC1]

### Synthetic Route:

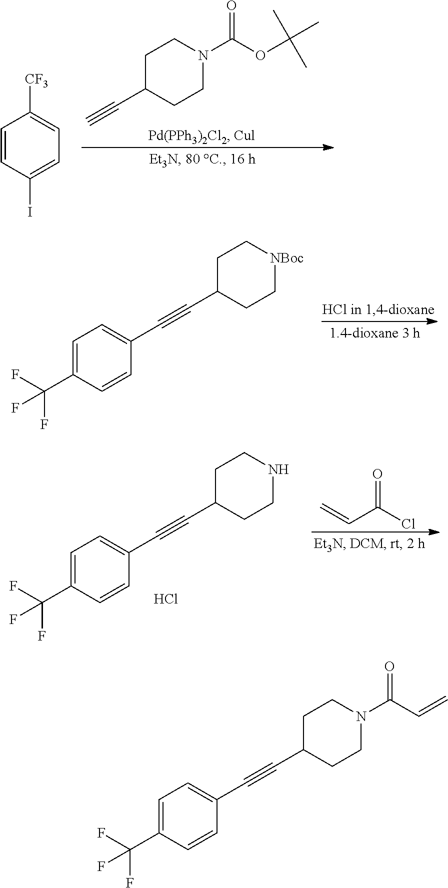

### Synthesis of tert-butyl 4-((4-(trifluoromethyl)phenyl)ethynyl)piperidine-1-carboxylate

1-Iodo-4-(trifluoromethyl)benzene (500 mg, 1.84 mmol), a stir bar, tert-butyl 4-ethynylpiperidine-1-carboxylate (461 mg, 2.20 mmol), Pd(PPh₃)₂Cl₂ (257 mg, 0.366 mmol), CuI (70 mg, 0.368 mmol) and Et₃N (8 mL) were added to a 40 mL sealed vial and stirred until homogeneous. The resulting mixture was stirred overnight at 80° C. under nitrogen atmosphere, then cooled to room temperature, concentrated under the reduced pressure. The residue was purified by silica gel chromatography (0%-30% EA in PE) to give tert-butyl 4-((4-(trifluoromethyl)phenyl) ethynyl)piperidine-1-carboxylate (600 mg, 92.4%) as a white solid. MS (ESI, m/z) calcd. for C₁₉H₂₂F₃NO₂, 353.16, found, 298.05 [M+H-^{t}Bu]⁺.

### Synthesis of 4-((4-(trifluoromethyl)phenyl)ethynyl)piperidine hydrogen chloride

4-((4-(Trifluoromethyl)phenyl)ethynyl)piperidine-1-carboxylate (300 mg, 0.849 mmol), a stir bar and 1,4-dioxane (2 mL) were added to a 25 mL round bottom flask and stirred until homogeneous, then treated with HCl in 1,4-dioxane (2 mL, 4M, 8 mmol) dropwise at 0° C. The resulting mixture was stirred for 3 hours at room temperature, concentrated under the reduced pressure to afford 4-((4-(trifluoromethyl)phenyl)ethynyl) piperidine hydrogen chloride (240 mg, crude) as a white solid. MS (ESI, m/z) calcd. for C₁₄H₁₄F₃N, 253.11, found, 254.00 [M+H]⁺.

### Synthesis of 1-(4-((4-(trifluoromethyl)phenyl)ethynyl)piperidin-1-yl)prop-2-en-1-one

4-((4-(Trifluoromethyl)phenyl)ethynyl)piperidine hydrochloride (120 mg, 0.414 mmol), a stir bar, Et₃N (143 mg, 1.41 mmol) and DCM (1.5 mL) were added to a 25 mL round bottom flask and stirred until homogeneous, then treated with a solution of acryloyl chloride (90 mg, 0.994 mmol) in DCM (0.5 mL) dropwise at 0° C. The resulting mixture was stirred until 2 hours at room temperature, diluted with water (5 mL) and extracted with DCM (10 mL×3). The combined organic layers were washed with brine, dried over anhydrous sodium sulfate, filtered and concentrated under vacuum pressure. The residue was purified by PREP-HPLC (Column: XBridge Prep OBD C18 Column, 19*250 mm, 5 μm; Mobile Phase A: Water (10 mmol/L NH4HCO3), Mobile Phase B: ACN; Flow rate: 25 mL/min; Gradient: 55% B to 85% B in 7 min, 85% B; Wave Length: 254 nm; RT1(min): 5.88; Injection Volume: 2 mL) to give 1-(4-((4-(trifluoromethyl) phenyl)ethynyl)piperidin-1-yl)prop-2-en-1-one as a colorless oil (29.7 mg, 23.0%). MS (ESI, m/z) calcd. for C₁₇H₁₆F₃NO, 307.12, found, 308.05 [M+H]⁺. ¹H NMR (400 MHz, DMSO-d₆) δ 7.75-7.69 (m, 2H), 7.66-7.60 (m, 2H), 6.82 (dd, J=16.7, 10.5 Hz, 1H), 6.10 (dd, J=16.7, 2.5 Hz, 1H), 5.67 (dd, J=10.5, 2.5 Hz, 1H), 3.94-3.79 (m, 2H), 3.47-3.36 (m, 1H), 3.31-3.23 (m, 1H), 3.06-2.96 (m, 1H), 1.91-1.85 (m, 2H), 1.60-1.55 (m, 2H); ¹⁹F NMR (376 MHz, DMSO-d₆) δ: −61.25

### Example 81: Synthesis of (R,E)-1-(3-(4-(trifluoromethyl)styryl)pyrrolidin-1-yl)but-2-yn-1-one

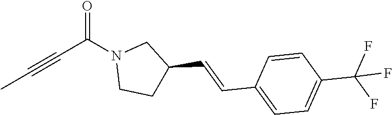 [SMILES: CC#CC(=O)N1CC[C@H](/C=C/c2ccc(C(F)(F)F)cc2)C1]

### Synthetic Route:

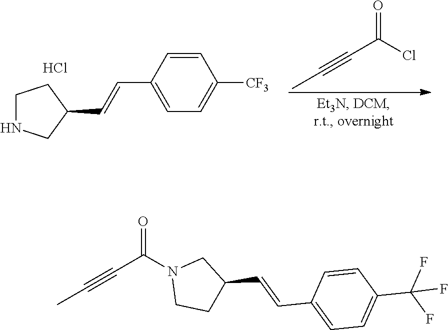

### Synthesis of (R, E)-1-(3-(4-(trifluoromethyl)styryl)pyrrolidin-1-yl)but-2-yn-1-one

(R, E)-3-(4-(Trifluoromethyl)styryl)pyrrolidine hydrochloride (350 mg, 1.260 mmol), Et₃N (638 mg, 6.30 mmol), a stir bar and DCM (3 mL) were added to a 20 mL vial and stirred until homogeneous, then treated with but-2-ynoyl chloride (194 mg, 1.89 mmol) in DCM (1 mL) dropwise at 0° C. The resulting mixture was stirred overnight at room temperature, then diluted with water (50 mL) and extracted with DCM (50 mL×2). The combined extracts were washed with brine, dried over anhydrous sodium sulfate, filtered and concentrated under the reduced pressure. The residue was purified by Prep-HPLC with (Column: Agilent ZORBAX SB-Aq, 21.2*250 mm 7 m; Mobile Phase A: Water (10 mmol/L NH4HCO3), Mobile Phase B: ACN; Flow rate: 25 mL/min; Gradient: 45% B to 65% B in 7 min, 65% B; Wave Length: 254 nm; RT1(min): 6.32) to afford (R, E)-1-(3-(4-(trifluoromethyl)styryl)pyrrolidin-1-yl)but-2-yn-1-one as a yellow solid (94.5 mg, 24.1%). MS (ESI, m/z) calcd. for C₁₇H₁₆F₃NO, 307.12, found, 308.05 [M+H]⁺. ¹H NMR (400 MHz, DMSO-d₆) δ 7.72-7.58 (m, 4H), 6.62 (dd, J=16.0, 8.4 Hz, 1H), 6.50 (dd, J=16.0, 7.3 Hz, 1H), 3.90-3.70 (m, 1H), 3.64-3.38 (m, 2H), 3.32-2.98 (m, 2H), 2.20-2.05 (m, 1H), 2.01 (d, J=4.3 Hz, 3H), 1.91-1.76 (m, 1H).

### Example 82: Synthesis of (R, E)-1-(3-(4-(trifluoromethyl)styryl)pyrrolidin-1-yl)prop-2-yn-1-one

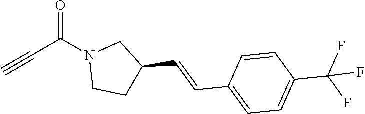 [SMILES: C#CC(=O)N1CC[C@H](/C=C/c2ccc(C(F)(F)F)cc2)C1]

### Synthetic Route:

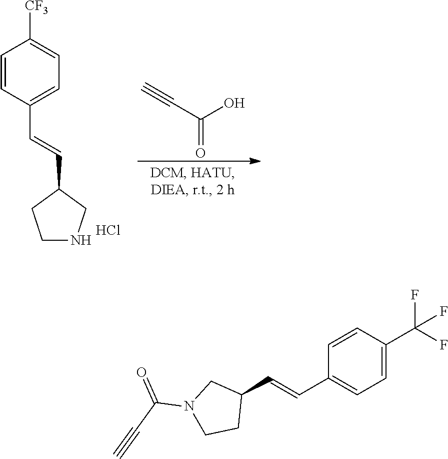

### Synthesis of (R, E)-1-(3-(4-(trifluoromethyl)styryl)pyrrolidin-1-yl)prop-2-yn-1-one

Propiolic acid (152 mg, 2.17 mmol), DIEA (559 mg, 4.33 mmol), (R, E)-3-(4-(Trifluoromethyl) styryl)pyrrolidine hydrochloride (400 mg, 1.44 mmol), a stir bar, DCM (15 mL) were added to a nitrogen-purged 100 mL round-bottom flask and stirred until homogeneous, then treated with HATU (823 mg, 0.271 mmol) in batches. The resulting mixture was stirred for 2 hours at room temperature then quenched with water (20 mL) and extracted with DCM (100 mL×2). The combined organic layers were dried over anhydrous sodium sulfate, filtered and concentrated under the reduced pressure. The residue was purified by reverse-phase chromatography with ACN/H₂O (0.1% FA) (5%-60%) to afford (R, E)-1-(3-(4-(trifluoromethyl)styryl)pyrrolidin-1-yl)prop-2-yn-1-one as a white solid (298 mg, 70.3% yield). MS (ESI, m/z) calcd. for C₁₆H₁₄F₃NO, 293.10, found, 294.05 [M+H]⁺. ¹H NMR (400 MHz, DMSO-d₆) δ 7.78-7.60 (m, 4H), 6.70-6.45 (m, 2H), 4.48 (d, J=2.1 Hz, 1H), 3.95-3.35 (m, 4H), 3.22-3.00 (m, 1H), 2.20-2.05 (m, 1H), 1.96-1.76 (m, 1H); ¹⁹F NMR (376 MHz, DMSO-d₆) δ: −60.84.

### Example 83: Synthesis of 1-((3S,4R)-3-(pyrimidin-2-ylamino)-4-((E)-4-(trifluoromethyl) styryl)pyrrolidin-1-yl)prop-2-en-1-one

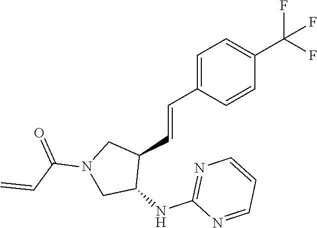 [SMILES: C=CC(=O)N1C[C@@H](/C=C/c2ccc(C(F)(F)F)cc2)[C@H](Nc2ncccn2)C1]

### Synthetic Route:

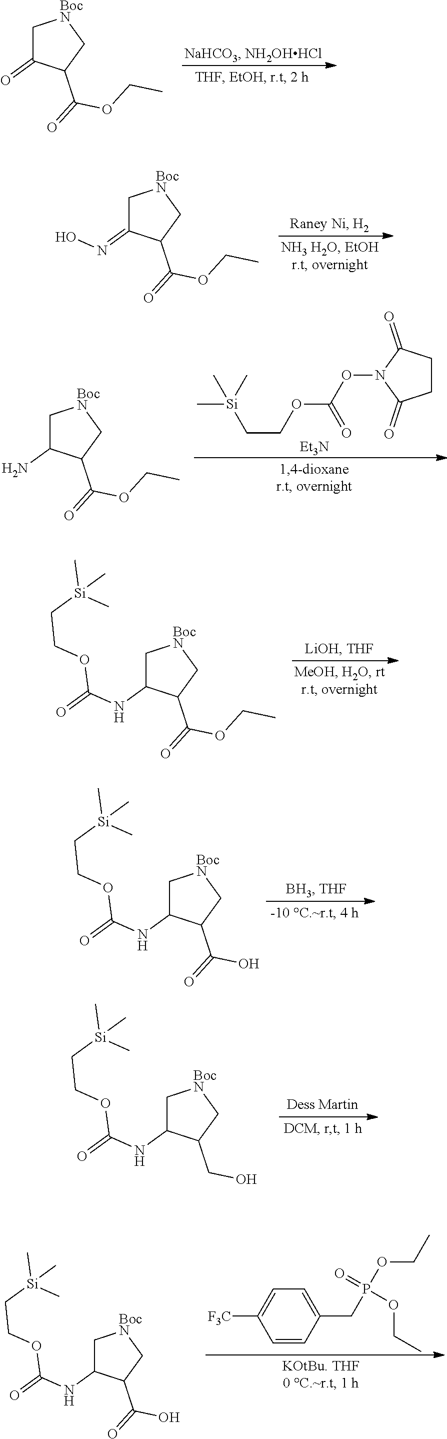

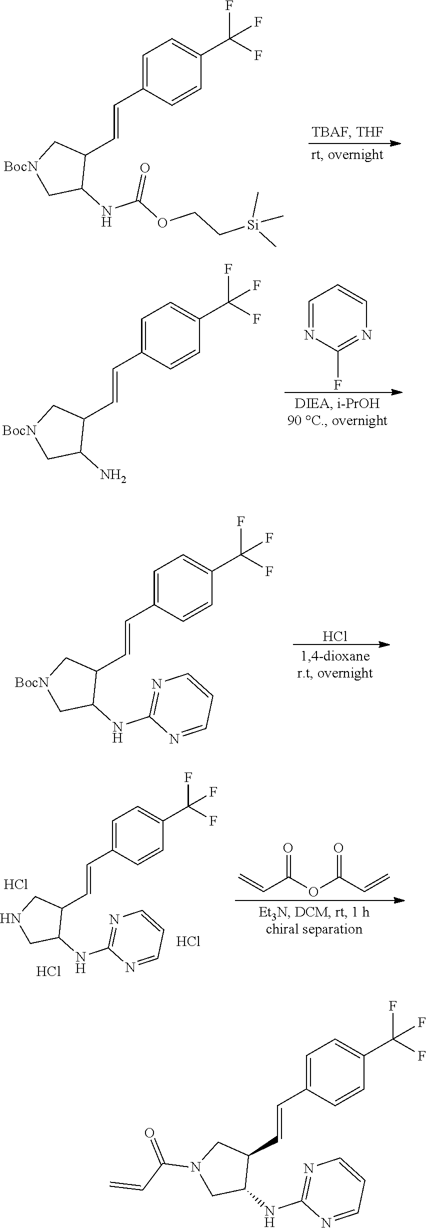

### Synthesis of 1-(tert-butyl) 3-ethyl (Z)-4-(hydroxyimino)pyrrolidine-1,3-dicarboxylate

1-(tert-Butyl) 3-ethyl 4-oxopyrrolidine-1,3-dicarboxylate (10 g, 38.9 mmol), sodium bicarbonate (4.9 g, 58.3 mmol), a stir bar, THF (50 mL) and EtOH (100 mL) were added to a 500 mL flask and stirred until homogenous, then treated with hydroxylamine hydrochloride (4.05 g, 58.3 mmol). The resulting mixture was stirred at room temperature for 2 hours. All solvents were removed under the reduced pressure. The residue obtained was diluted with H₂O and extracted with EA. The organic layers were combined, dried over anhydrous Na₂SO₄, filtered and concentrated to afford 1-tert-butyl 3-ethyl (4Z)-4-(hydroxyimino) pyrrolidine-1,3-dicarboxylate (9.7 g, 91.7%) as a yellow oil. MS (ESI, m/z) calcd. for C₁₂H₂₀N₂O₅, 272.14, found: 217.00 [M-^{t}Bu+H]⁺.

### Synthesis of 1-(tert-butyl) 3-ethyl 4-aminopyrrolidine-1,3-dicarboxylate

1-tert-Butyl 3-ethyl (4Z)-4-(hydroxyimino) pyrrolidine-1,3-dicarboxylate (9.7 g, 35.6 mmol), a stir bar, ammonium hydroxide (10 mL) and EtOH (100 mL) were added to a 250 mL flask and stirred until homogenous, then treated with Raney Ni (10 g). The resulting mixture was maintained under hydrogen and stirred at room temperature overnight. The reaction was filtered and concentrated. The residue obtained was purified by silica gel chromatography (MeOH/DCM=0~10%) to afford 1-(tert-butyl) 3-ethyl 4-aminopyrrolidine-1,3-dicarboxylate (1.3 g, 14.1%) as a yellow oil. MS (ESI, m/z) calcd. for C₁₂H₂₂N₂O₄: 258.16, found: 259.10 [M+H]_.

### Synthesis of 1-(tert-butyl) 3-ethyl 4-(((2-(trimethylsilyl)ethoxy)carbonyl)amino)pyrrolidine-1,3-dicarboxylate

1-(tert-Butyl) 3-ethyl 4-aminopyrrolidine-1,3-dicarboxylate (1.3 g, 5.03 mmol), triethylamine (1.0 g, 9.88 mmol), a stir bar and 1,4-dioxane (25 mL) were added to a 100 mL flask and stirred until homogenous, then treated with 2,5-dioxopyrrolidin-1-yl 2-(trimethylsilyl)ethyl carbonate (1.3 g, 5.01 mmol). The resulting mixture was stirred at room temperature overnight. The reaction was diluted with DCM and washed twice with an aqueous NaHCO₃ saturated solution. The organic layers were dried over anhydrous Na₂SO₄, filtered and concentrated. The residue obtained was purified by silica gel chromatography (EA/PE=0~25%) to afford 1-(tert-butyl) 3-ethyl 4-(((2-(trimethylsilyl)ethoxy)carbonyl)amino)pyrrolidine-1,3-dicarboxylate (1.7 g, 83.9%) as a colorless oil. MS (ESI, m/z) calcd. for C₁₈H₃₄N₂O₆Si, 402.22, found: 401.10 [M−H]⁺.

### Synthesis of 1-(tert-butoxycarbonyl)-4-(((2-(trimethylsilyl)ethoxy)carbonyl)amino) pyrrolidine-3-carboxylic acid

1-(tert-Butyl) 3-ethyl 4-(((2-(trimethylsilyl)ethoxy)carbonyl)amino)pyrrolidine-1,3-dicarboxylate (1.7 g, 4.22 mmol), a stir bar, EtOH (10 mL) and THF (30 mL) were added to a 250 mL flask and stirred until homogenous, then treated with lithium hydroxide aqueous solution (5.6 mL, 16.8 mmol, 3 M in H₂O). The resulting mixture was stirred at room temperature overnight. All solvents were removed under the reduced pressure. The residue obtained was purified by silica gel (MeOH/DCM=0-20%) to afford 1-(tert-butoxycarbonyl)-4-(((2-(trimethylsilyl)ethoxy) carbonyl)amino)pyrrolidine-3-carboxylic acid (1.5 g, 94.8%) as an off-white solid. MS (ESI, m/z) calcd. for C₁₆H₃₀N₂O₆Si: 374.19, found, 373.05 [M−H]⁺.

### Synthesis of tert-butyl 3-(hydroxymethyl)-4-(((2-(trimethylsilyl)ethoxy)carbonyl)amino) pyrrolidine-1-carboxylate

1-(tert-Butoxycarbonyl)-4-(((2-(trimethylsilyl)ethoxy)carbonyl)amino)pyrrolidine-3-carboxylic acid (2.2 g, 5.87 mmol), a stir bar and tetrahydrofuran (10 mL) were added to a 100 mL flask and stirred until homogenous, then treated with borane (18 mL, 18.0 mmol, 1 M in THF) dropwise at −10° C. The resulting mixture was stirred at −10° C. for 20 min, then warmed to room temperature and continued to stir for 4 hours. The mixture was quenched with MeOH and concentrated under the reduced pressure. The residue obtained was purified by silica gel chromatography (EA/PE=0~65%) to afford tert-butyl 3-(hydroxymethyl)-4-(((2-(trimethylsilyl)ethoxy)carbonyl) amino)pyrrolidine-1-carboxylate (590 mg, 27.9%) as a yellow oil. MS (ESI, m/z) calcd. for C₁₆H₃₂N₂O₅Si, 360.21, found, 359.05 [M−H]⁺.

### Synthesis of tert-butyl 3-formyl-4-(((2-(trimethylsilyl)ethoxy)carbonyl)amino)pyrrolidine-1-carboxylate

tert-Butyl 3-(hydroxymethyl)-4-(((2-(trimethylsilyl)ethoxy)carbonyl)amino)pyrrolidine-1-carboxylate (570 mg, 1.58 mmol), a stir bar and DCM (15 mL) were added to a 100 mL flask and stirred until homogenous, then treated with Dess-Martin periodinane (670 mg, 1.58 mmol). The resulting mixture was stirred at room temperature for 1 hour. The reaction was quenched with NaHCO₃ solution and extracted with DCM. The organic layers were combined, dried over anhydrous Na₂SO₄, filtered and concentrated. The residue obtained was purified by silica gel chromatography (EA/PE=0-50%) to afford tert-butyl 3-(hydroxymethyl)-4-(((2-(trimethylsilyl)ethoxy)carbonyl)amino)pyrrolidine-1-carboxylate (470 mg, 82.9%) as a yellow oil. MS (ESI, m/z) calcd. for C₁₆H₃₀N₂O₅Si, 358.19, found: 357.00 [M−H]⁺.

### Synthesis of tert-butyl (E)-3-(4-(trifluoromethyl)styryl)-4-(((2-(trimethylsilyl)ethoxy) carbonyl)amino)pyrrolidine-1-carboxylate

tert-Butyl 3-(hydroxymethyl)-4-(((2-(trimethylsilyl)ethoxy)carbonyl)amino)pyrrolidine-1-carboxylate (450 mg, 1.26 mmol), diethyl (4-(trifluoromethyl)phenyl)methylphosphonate (448 mg, 1.51 mmol), a stir bar and THF (10 mL) were added to a 100 mL flask and stirred until homogenous, then treated with potassium tert-butoxide (211 mg, 1.88 mmol) in portions at 0° C. The resulting mixture was stirred at room temperature for 1 hour. The reaction was quenched with H₂O and extracted with EA. The organic layers were combined, dried over anhydrous Na₂SO₄, filtered and concentrated. The residue obtained was purified by silica gel chromatography (EA/PE=0~25%) to afford tert-butyl (E)-3-(4-(trifluoromethyl)styryl)-4-(((2-(trimethylsilyl)ethoxy)carbonyl) amino)pyrrolidine-1-carboxylate (500 mg, 79.6%) as a colorless semi-solid. MS (ESI, m/z) calcd. for C₂₄H₃₅F₃N₂O₄Si, 500.23, found: 523.15 [M+Na]⁺.

### Synthesis of tert-butyl (E)-3-amino-4-(4-(trifluoromethyl)styryl)pyrrolidine-1-carboxylate

tert-Butyl (E)-3-(4-(trifluoromethyl)styryl)-4-(((2-(trimethylsilyl)ethoxy)carbonyl)amino) pyrrolidine-1-carboxylate (480 mg, 0.959 mmol), a stir bar and THF (10 mL) were added to a 100 mL flask and stirred until homogenous, then treated with TBAF (2.9 mL, 2.90 mmol, 1 M in THF). The resulting mixture was stirred at room temperature overnight. The reaction was quenched with NaHCO₃ solution and extracted with EA. The organic layers were combined, dried over anhydrous Na₂SO₄, filtered and concentrated to afford tert-butyl (E)-3-amino-4-(4-(trifluoromethyl)styryl) pyrrolidine-1-carboxylate (340 mg, 99.5%) as a yellow oil. MS (ESI, m/z) calcd. for C₁₈H₂₃F₃N₂O₂, 356.17, found: 301.00 [M-^{t}Bu+H]⁺.

### Synthesis of tert-butyl (E)-3-(pyrimidin-2-ylamino)-4-(4-(trifluoromethyl)styryl) pyrrolidine-1-carboxylate

tert-Butyl (E)-3-amino-4-(4-(trifluoromethyl)styryl)pyrrolidine-1-carboxylate (340 mg, 0.954 mmol), 2-fluoropyrimidine (112 mg, 1.14 mmol), a stir bar and i-PrOH (8 mL) were added to a 100 mL flask and stirred until homogenous, then treated with DIEA (247 mg, 1.91 mmol). The resulting mixture was stirred at 90° C. overnight. The reaction was concentrated. The residue obtained was purified by silica gel chromatography (EA/PE=0-45%) to afford tert-butyl (E)-3-(pyrimidin-2-ylamino)-4-(4-(trifluoromethyl) styryl)pyrrolidine-1-carboxylate (258 mg, 62.3%) as a yellow solid. MS (ESI, m/z) calcd. for C₂₂H₂₅F₃N₄O₂, 434.19, found, 435.15 [M+H]⁺.

### Synthesis of (E)-N-(4-(4-(trifluoromethyl)styryl)pyrrolidin-3-yl)pyrimidin-2-amine trihydrochloride

tert-butyl (E)-3-(pyrimidin-2-ylamino)-4-(4-(trifluoromethyl)styryl)pyrrolidine-1-carboxylate (258 mg, 0.594 mmol), a stir bar and 1,4-dioxane (8 mL) were added to a 100 mL flask and stirred until homogenous, then treated with hydrogen chloride (3 mL, 12.0 mmol, 4 M in 1,4-dioxane). The resulting mixture was stirred at room temperature overnight. The reaction was concentrated to afford (E)-N-(4-(4-(trifluoromethyl)styryl)pyrrolidin-3-yl)pyrimidin-2-amine trihydrochloride (260 mg, 98.7%) as an off-white solid. MS (ESI, m/z) calcd. for C₁₇H₁₇F₃N₄, 334.14, found: 335.10 [M+H]⁺.

### Synthesis of 1-((3S,4R)-3-(pyrimidin-2-ylamino)-4-((E)-4-(trifluoromethyl)styryl) pyrrolidin-1-yl)prop-2-en-1-one

(E)-N-(4-(4-(Trifluoromethyl)styryl)pyrrolidin-3-yl)pyrimidin-2-amine trihydrochloride (250 mg, 0.563 mmol), triethylamine (342 mg, 3.38 mmol), a stir bar and DCM (3 mL) were added to a 8 mL vial and stirred until homogenous, then treated with a solution of prop-2-enoyl prop-2-enoate (85 mg, 0.056 mmol) in DCM (1 mL) dropwise. The resulting mixture was stirred at room temperature for 1 hour. The reaction was quenched with H₂O and extracted with DCM. The organic layers were combined, dried over anhydrous Na₂SO₄, filtered and concentrated. The residue obtained was purified by Prep-HPLC (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 32% B to 62% B in 7 min, 62% B; Wave Length: 254 nm; RT1(min): 5.82; Injection Volume: 1.5 mL) and Prep-Chiral-HPLC (Column: CHIRAL ART Cellulose-SB, 4.6*100 mm, 3.0 um; Mobile Phase A: MtBE (0.1% DEA):EtOH=93:7; Flow rate: 1 mL/min; Gradient: isocratic; Injection Volume: 5 ul mL; RT1(min): 4.02; RT2(min): 4.55) to give three products. The first eluting fractions were lyophilized to afford 1-((3S,4R)-3-(pyrimidin-2-ylamino)-4-((E)-4-(trifluoromethyl) styryl)pyrrolidin-1-yl)prop-2-en-1-one (27.2 mg, 12.4%) as a white solid. MS (ESI, m/z) calcd. for C₂₀H₁₉F₃N₄O, 388.15, found, 389.15 [M+H]⁺. ¹H NMR (400 MHz, DMSO-d₆) δ 8.28 (dd, J=4.8, 2.7 Hz, 2H), 7.88-7.32 (m, 5H), 6.76-6.35 (m, 4H), 6.16 (dt, J=16.8, 2.7 Hz, 1H), 5.74-5.63 (m, 1H), 4.54-4.39 (m, 1H), 4.13-3.79 (m, 2H), 3.62-3.36 (m, 2H), 3.25-3.04 (m, 1H); ¹⁹F NMR (376 MHz, DMSO-d₆) δ: −60.84.

### Example 84: Synthesis of 1-((3R,4S)-3-(pyrimidin-2-ylamino)-4-((E)-4-(trifluoromethyl) styryl)pyrrolidin-1-yl)prop-2-en-1-one

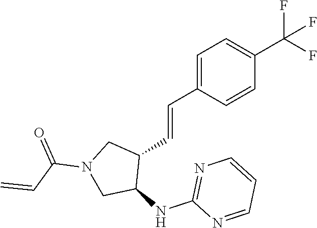 [SMILES: C=CC(=O)N1C[C@H](Nc2ncccn2)[C@@H](/C=C/c2ccc(C(F)(F)F)cc2)C1]

### Synthetic Route:

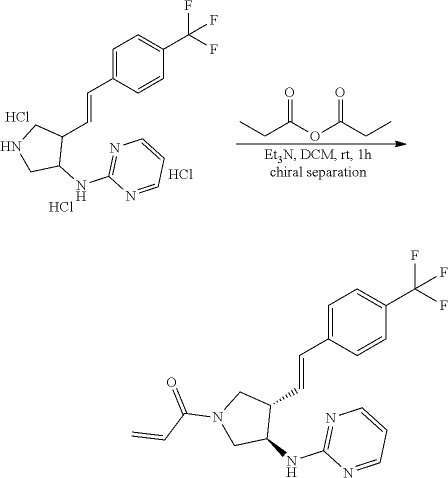

### Synthesis of 1-((3R,4S)-3-(pyrimidin-2-ylamino)-4-((E)-4-(trifluoromethyl)styryl)pyrrolidin-1-yl)prop-2-en-1-one

(E)-N-(4-(4-(trifluoromethyl)styryl)pyrrolidin-3-yl)pyrimidin-2-amine trihydrochloride (250 mg, 0.563 mmol), triethylamine (342 mg, 3.38 mmol), a stir bar and DCM (3 mL) were added to a 8 mL vial and stirred until homogenous, then treated with a solution of prop-2-enoyl prop-2-enoate (85 mg, 0.056 mmol) in DCM (1 mL) dropwise. The resulting mixture was stirred at room temperature for 1 hour. The reaction was quenched with H₂O and extracted with DCM. The organic layers were combined, dried over anhydrous Na₂SO₄, filtered and concentrated. The residue obtained was purified by Prep-HPLC (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 32% B to 62% B in 7 min, 62% B; Wave Length: 254 nm; RT1(min): 5.82; Injection Volume: 1.5 mL) and Prep-Chiral-HPLC (Column: CHIRAL ART Cellulose-SB, 4.6*100 mm, 3.0 um; Mobile Phase A: MtBE (0.1% DEA):EtOH=93:7; Flow rate: 1 mL/min; Gradient: isocratic; Injection Volume: 5 ul mL; RT1(min): 4.02; RT2(min): 4.55) to give three products. The second eluting fractions were lyophilized to afford 1-((3R,4S)-3-(pyrimidin-2-ylamino)-4-((E)-4-(trifluoromethyl)styryl)pyrrolidin-1-yl)prop-2-en-1-one (27.2 mg, 12.4%) as a white solid. MS (ESI, m/z) calcd. for C₂₀H₁₉F₃N₄O, 388.15, found: 389.15 [M+H]⁺. ¹H NMR (400 MHz, DMSO-d₆) δ 8.28 (dd, J=4.8, 2.7 Hz, 2H), 7.73-7.56 (m, 4H), 7.52 (dd, J=16.5, 7.9 Hz, 1H), 6.72-6.53 (m, 3H), 6.48 (dd, J=15.9, 7.9 Hz, 1H), 6.16 (dt, J=16.8, 2.8 Hz, 1H), 5.78-5.61 (m, 1H), 4.55-4.36 (m, 1H), 4.14-3.77 (m, 2H), 3.63-3.35 (m, 2H), 3.25-3.05 (m, 1H); ¹⁹F NMR (376 MHz, DMSO-d₆) δ: −60.84.

### Example 85: Synthesis of (S, E)-1-(3-(3-(4-(trifluoromethyl)phenyl)allyl)pyrrolidin-1-yl)prop-2-en-1-one

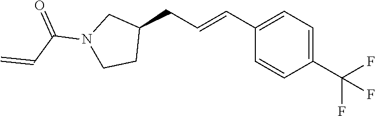 [SMILES: C=CC(=O)N1CC[C@H](C/C=C/c2ccc(C(F)(F)F)cc2)C1]

### Synthetic Route:

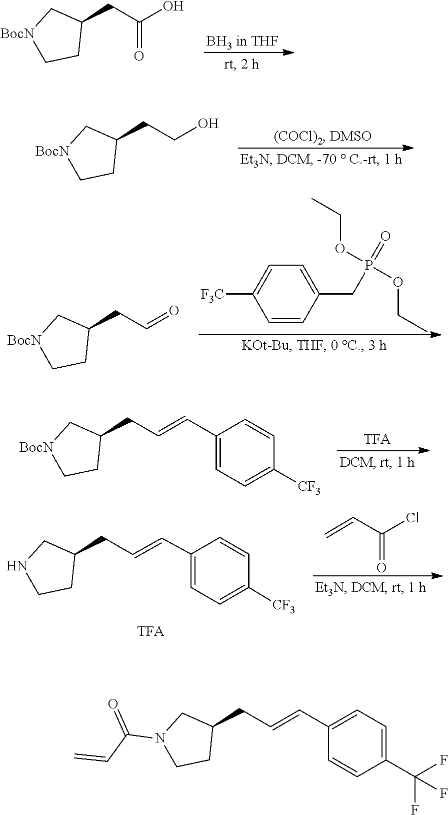

### Synthesis of tert-butyl (R)-3-(2-hydroxyethyl)pyrrolidine-1-carboxylate

(R)-2-(1-(tert-Butoxycarbonyl)pyrrolidin-3-yl)acetic acid (2 g, 8.72 mmol), a stir bar and BH₃-THF (30 mL) were added to 100 mL round-bottom flask and stirred until homogenous. The resulting mixture was stirred for 2 hours at room temperature, then quenched with MeOH. The resulting mixture was extracted with EA (60 mL×2). The combined organic layers were washed with brine, dried over anhydrous sodium sulfate, filtered and concentrated under the reduced pressure. The residue obtained was purified by silica gel chromatography (0-50% EA/PE) to afford tert-butyl (R)-3-(2-hydroxyethyl)pyrrolidine-1-carboxylate (1.6 g, 85.2%) as a colorless oil. MS (ESI, m/z) calcd. for C₁₁H₂₁NO₃, 215.15, found, 160.20 [M+H-56]⁺.

### Synthesis of tert-butyl (R)-3-(2-oxoethyl)pyrrolidine-1-carboxylate

Oxalyl chloride (1.18 g, 9.29 mmol), a stir bar and DCM (10 mL) were added to an oven-dried and nitrogen-purged 40 mL flask and stirred until homogenous, then treated with a solution of DMSO (1.45 g, 18.6 mmol) in DCM (5 mL) at −70° C. under nitrogen atmosphere. After stirring for 30 min, tert-butyl (R)-3-(2-hydroxyethyl)pyrrolidine-1-carboxylate (1 g, 4.65 mmol) in DCM (5 mL) was added dropwise and stirring continued for 30 min. To the mixture was added Et₃N (3.76 g, 37.2 mmol) and the resulting mixture was stirred at −70° C. for 30 min, then warmed to room temperature and stirred for 1 hour, then diluted with DCM. The resulting mixture was extracted with DCM (10 mL×2). The combined organic layers were washed with brine, dried over anhydrous sodium sulfate, filtered and concentrated under the reduced pressure. The residue obtained was purified by silica gel chromatography (0-50% EA/PE) to afford tert-butyl (R)-3-(2-oxoethyl)pyrrolidine-1-carboxylate (950 mg, 95.9%) as a yellow oil. MS (ESI, m/z) calcd. for C₁₁H₁₉NO₃, 213.14, found, 158.05 [M+H-^{t}Bu]⁺.

### Synthesis of tert-butyl (S,E)-3-(3-(4-(trifluoromethyl)phenyl)allyl)pyrrolidine-1-carboxylate

tert-Butyl (R)-3-(2-oxoethyl)pyrrolidine-1-carboxylate (1 g, 4.69 mmol), K₂CO₃ (0.79 g, 7.03 mmol), a stir bar and THF (15 mL) were added to 40 mL vial and stirred until homogenous then treated with diethyl (4-(trifluoromethyl)phenyl)methylphosphonate (1.39 g. 4.69 mmol) at 0° C. The resulting mixture was stirred for 3 hours at 0° C., then quenched with water. The resulting mixture was extracted with EA (40 mL×2). The combined organic layers were washed with brine, dried over anhydrous sodium sulfate, filtered and concentrated under the reduced pressure. The residue obtained was purified by silica gel chromatography (0-50% EA/PE) to afford tert-butyl (S,E)-3-(3-(4-(trifluoromethyl)phenyl)allyl)pyrrolidine-1-carboxylate (1.2 g, 72.0%) as a white solid. MS (ESI, m/z) calcd. for C₁₉H₂₄F₃NO₂, 355.18, found, 300.15 [M+H-^{t}Bu]⁺.

### Synthesis of (S,E)-3-(3-(4-(trifluoromethyl)phenyl)allyl)pyrrolidine 2,2,2-trifluoroacetate

tert-Butyl (S,E)-3-(3-(4-(trifluoromethyl)phenyl)allyl)pyrrolidine-1-carboxylate (400 mg, 1.13 mmol), a stir bar and DCM (6 mL) were added to 20 mL vial and stirred until homogenous then treated with trifluoroacetic acid (1.2 mL) at room temperature. The resulting mixture was stirred for 1 h at room temperature, then concentrated under the reduced pressure to afford (S,E)-3-(3-(4-(trifluoromethyl)phenyl)allyl)pyrrolidine 2,2,2-trifluoroacetate (523 mg, crude) as a light yellow oil. MS (ESI, m/z) calcd. for C₁₆H₁₇F₆NO₂, 255.12, found, 256.10 [M+H]⁺.

### Synthesis of (S,E)-1-(3-(3-(4-(trifluoromethyl)phenyl)allyl)pyrrolidin-1-yl)prop-2-en-1-one

(S,E)-3-(3-(4-(Trifluoromethyl)phenyl)allyl)pyrrolidine 2,2,2-trifluoroacetate (523 mg, 1.42 mmol), DCM (6 mL), TEA (860 mg, 8.50 mmol) and a stir bar were added to a 20 mL vial and stirred until homogenous, then treated with acrylic anhydride (215 mg, 1.71 mmol) at 0° C. The resulting mixture was stirred for 1 hour at room temperature, then diluted with water (50 mL) and extracted with DCM (50 mL×2). The combined organic layers were washed with brine, dried over anhydrous sodium sulfate, filtered and concentrated. The resulting residue was purified by Prep-HPLC with (Column: XSelect CSH Fluoro Phenyl 5 m, 19 mm×250 mm; Mobile Phase A: Water (0.10% FA), Mobile Phase B: ACN; Flow rate: 25 mL/min mL/min; Gradient: 40% B to 57% B in 10 min; Wave Length: 254 nm/220 nm nm; RT1(min): 8.96) to afford (S,E)-1-(3-(3-(4-(trifluoromethyl)phenyl)allyl)pyrrolidin-1-yl)prop-2-en-1-one as a colorless oil (101 mg, 22.9% yield). MS (ESI, m/z) calcd. for C₁₇H₁₈F₃NO, 309.15, found, 310.10 [M+H]. ¹H NMR (400 MHz, DMSO-d₆) δ 7.70-7.60 (m, 4H), 6.62-6.47 (m, 3H), 6.16-6.06 (m, 1H), 5.69-5.60 (m, 1H), 3.80-3.45 (m, 3H), 3.23-2.98 (m, 1H), 2.42-2.24 (m, 3H), 2.15-1.96 (m, 1H), 1.72-1.49 (m, 1H); ¹⁹F NMR (376 MHz, DMSO-d₆) δ −60.81.

### Example 86: Synthesis of (R, E)-1-(3-(2-methyl-4-(4-(trifluoromethyl)phenyl)but-3-en-2-yl)pyrrolidin-1-yl)prop-2-en-1-one

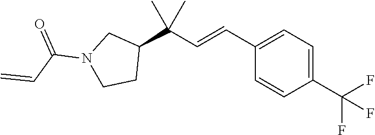 [SMILES: C=CC(=O)N1CC[C@H](C(C)(C)/C=C/c2ccc(C(F)(F)F)cc2)C1]

### Synthetic Route:

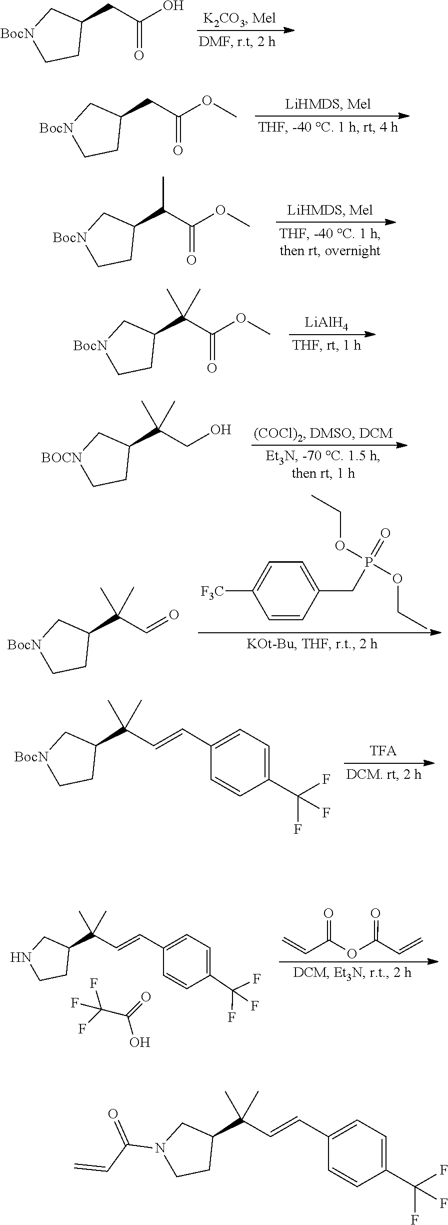

### Synthesis of tert-butyl (R)-3-(2-methoxy-2-oxoethyl)pyrrolidine-1-carboxylate

(R)-2-(1-(tert-Butoxycarbonyl)pyrrolidin-3-yl)acetic acid (4.0 g, 17.4 mmol), a stir bar, methyl iodide (2.97 g, 20.9 mmol) and DMF (40 mL) were added to a 250 mL round-bottom flask and stirred until homogeneous, then treated with potassium carbonate (4.86 g, 34.9 mmol). The resulting mixture was stirred at room temperature for 2 hours. The resulting solution was extracted with ethyl acetate (100 mL×3). The combined organic layers were washed with brine, dried over anhydrous sodium sulfate, filtered and concentrated under the reduced pressure. The residue obtained was purified by silica gel chromatography (EA/PE=0-50%) to afford tert-butyl (R)-3-(2-methoxy-2-oxoethyl)pyrrolidine-1-carboxylate as a colorless oil (4.1 g, 96.6% yield). MS (ESI, m/z) calcd. for C₁₂H₂₁NO₄, 243.14, found 188.05 [M+H-^{t}Bu]⁺.

### Synthesis of tert-butyl (R)-3-(1-methoxy-1-oxopropan-2-yl)pyrrolidine-1-carboxylate

tert-Butyl (R)-3-(2-methoxy-2-oxoethyl)pyrrolidine-1-carboxylate as a colorless oil (1.7 g, 6.99 mmol), a stir bar, methyl iodide (2.48 g, 17.5 mmol) and THF (30 mL) were added to a 100 mL round-bottom flask and stirred until homogeneous, then treated with LiHMDS (10.5 mL, 10.5 mmol, 1 mol/L) at −40° C. The reaction mixture was stirred at −40° C. for 1 hour, then stirred at room temperature for 4 hours under N2 atmosphere. The resulting solution was diluted with 40 mL of water, then extracted with EA (100 mL×3). The organic layers were combined, dried over Na₂SO₄, filtered and concentrated under the reduced pressure. The residue obtained was purified by silica gel chromatography (EA/PE=0-50%) to afford tert-butyl (R)-3-(1-methoxy-1-oxopropan-2-yl)pyrrolidine-1-carboxylate as a colorless oil (1.4 g, 77.9%). MS (ESI, m/z) calcd. for C₁₃H₂₃NO₄, 257.16, found 202.10 [M+H-^{t}Bu]⁺.

### Synthesis of tert-butyl (R)-3-(1-methoxy-2-methyl-1-oxopropan-2-yl)pyrrolidine-1-carboxylate

tert-Butyl (R)-3-(1-methoxy-1-oxopropan-2-yl)pyrrolidine-1-carboxylate (1.0 g, 6.99 mmol), a stir bar, methyl iodide (2.76 g, 19.4 mmol) and THF (30 mL) were added to a 100 mL round-bottom flask and stirred until homogeneous, then treated with LiHMDS (11.7 mL, 11.7 mmol, 1M) at −40° C. The reaction mixture was stirred at −40° C. for 1 hour, then stirred overnight at room temperature under nitrogen atmosphere. The resulting solution was quenched with 40 mL of water, then extracted with EA (100 mL×3). The organic layers were combined, dried over Na₂SO₄, filtered and concentrated under the reduced pressure. The residue was purified by silica gel chromatography (EA/PE=0-50%) to afford tert-butyl (R)-3-(1-methoxy-2-methyl-1-oxopropan-2-yl)pyrrolidine-1-carboxylate as a colorless oil (1.25 g, 59.3% yield). MS (ESI, m/z) calcd. for C₁₄H₂₅NO₄, 271.17, found 216.05 [M+H-^{t}Bu]⁺.

### Synthesis of tert-butyl (R)-3-(1-hydroxy-2-methylpropan-2-yl)pyrrolidine-1-carboxylate

tert-Butyl (R)-3-(1-methoxy-2-methyl-1-oxopropan-2-yl)pyrrolidine-1-carboxylate (1.25 g, 4.61 mmol), a stir bar and THF (20 mL) were added to a 100 mL round-bottom flask and stirred until homogeneous, then treated with LiAlH₄ (6.91 mL, 6.91 mmol) at 0° C. The resulting mixture was stirred at room temperature for 1 hour under nitrogen atmosphere. The resulting solution was quenched with 0.5 mL of water, then filtered with THF (60 mL). The organic layers were combined, dried over Na₂SO₄, filtered and concentrated under the reduced pressure. The residue obtained was purified by silica gel chromatography (EA/PE=0-60%) to afford tert-butyl (R)-3-(1-hydroxy-2-methylpropan-2-yl)pyrrolidine-1-carboxylate as a colorless oil (500 mg, 44.6% yield). MS (ESI, m/z) mass calcd. for C₁₃H₂₅NO₃, 243.18, found 188.05 [M+H-^{t}Bu]⁺.

### Synthesis of tert-butyl (R)-3-(2-methyl-1-oxopropan-2-yl)pyrrolidine-1-carboxylate

Oxalyl chloride (456 mg, 3.59 mmol), a stir bar and DCM (6 mL) were added to an oven-dried and nitrogen-purged 50 mL round-bottom flask and stirred until homogenous, then treated with a solution of DMSO (565 mg, 7.23 mmol) in DCM (1 mL) at −70° C. under nitrogen atmosphere. After stirring for 30 min, tert-butyl (R)-3-(1-hydroxy-2-methylpropan-2-yl)pyrrolidine-1-carboxylate (440 mg, 1.81 mmol) in DCM (3 mL) was added dropwise and stirring continued for 30 min. To the mixture was added Et₃N (1.46 g, 14.5 mmol) dropwise and the resulting mixture was stirred at −70° C. for 30 min, then warmed to room temperature and stirred for 1 hour. The mixture was quenched with water (15 mL) and extracted with DCM (50 mL×3). The combined organic extracts were washed with brine, dried over anhydrous Na₂SO₄, filtered and concentrated under the reduced pressure to afford tert-butyl (R)-3-(2-methyl-1-oxopropan-2-yl)pyrrolidine-1-carboxylate as a yellow solid (415 mg, 95.1% yield). MS (ESI, m/z) calcd. for C₁₃H₂₃NO₃, 241.16, found, 186.10 [M+H-^{t}Bu]⁺

### Synthesis of tert-butyl (R,E)-3-(2-methyl-4-(4-(trifluoromethyl)phenyl)but-3-en-2-yl) pyrrolidine-1-carboxylate

Diethyl (4-(trifluoromethyl)phenyl)methylphosphonate (510 mg, 1.72 mmol), a stir bar, THF (7 mL) were added to a 40 mL vial and stirred until homogeneous before the reaction vessel was cooled to 0° C., then treated with potassium tert-butoxide (415 mg, 1.72 mmol) and a solution of tert-butyl (R)-3-(2-methyl-1-oxopropan-2-yl)pyrrolidine-1-carboxylate (289 mg, 2.58 mmol) in THF (3 mL). The resulting mixture was stirred at room temperature for 2 hours then quenched with water (10 mL) and extracted with EA (50 mL×3). The combined organic extracts were washed with brine, dried over anhydrous Na₂SO₄, filtered and concentrated under the reduced pressure. The residue was purified by silica gel chromatography (EA/PE=0~20%) to afford tert-butyl (R,E)-3-(2-methyl-4-(4-(trifluoromethyl)phenyl)but-3-en-2-yl) pyrrolidine-1-carboxylate as a yellow oil (590 mg. 89.5% yield). MS (ESI, m/z) calcd. for C₂₁H₂₈F₃NO₂, 383.20, found, 328.05 [M+H-^{t}Bu]⁺.

### Synthesis of (R,E)-3-(2-methyl-4-(4-(trifluoromethyl)phenyl)but-3-en-2-yl)pyrrolidine 2,2,2-trifluoroacetate

tert-Butyl (R,E)-3-(2-methyl-4-(4-(trifluoromethyl)phenyl)but-3-en-2-yl) pyrrolidine-1-carboxylate (570 mg, 1.49 mmol), a stir bar, DCM (10 mL) were added to a 100 mL round-bottom flask and stirred until homogenous then treated with TFA (4 mL). The resulting mixture was stirred at room temperature for 2 hours, then concentrated under the reduced pressure to afford (R,E)-3-(2-methyl-4-(4-(trifluoromethyl) phenyl)but-3-en-2-yl)pyrrolidine 2,2,2-trifluoroacetate as a brown solid (700 mg, crude). MS (ESI, m/z) calcd. for C₁₆H₂₀F₃N, 283.20, found, 284.10 [M+H]⁺.

### Synthesis of (R, E)-1-(3-(2-methyl-4-(4-(trifluoromethyl)phenyl)but-3-en-2-yl)pyrrolidin-1-yl)prop-2-en-1-one

(R,E)-3-(2-Methyl-4-(4-(trifluoromethyl) phenyl)but-3-en-2-yl)pyrrolidine 2,2,2-trifluoroacetate (650 mg, 1.27 mmol), a stir bar, DCM (8 mL) were added to a 40 mL vial and stirred until homogeneous before the reaction vessel was cooled to 0° C. then treated with Et₃N (642 mg, 6.34 mmol) and a solution of prop-2-enoyl prop-2-enoate (240 mg, 1.90 mmol) in DCM (2 mL). The resulting mixture was stirred at room temperature for 2 hours, then quenched with water (10 mL) and extracted with DCM (50 mL×3). The combined organic extracts were washed with brine, dried over anhydrous Na₂SO₄, filtered and concentrated under the reduced pressure. The residue was purified by reverse-phase chromatography with ACN/H₂O (0.05% NH₄HCO₃) (5%-60%) to afford a crude product, which was further purified by PREP_HPLC_MC3 (Column: XBridge C18 5 m, 19 mm×250 mm; Mobile Phase A: Water (10 mmol/L NH₄HCO₃), Mobile Phase B: ACN; Flow rate: 25 mL/min mL/min; Gradient: 56% B to 86% B in 7 min; Wave Length: 254 nm/220 nm nm; RT1(min): 6.23) to afford (R,E)-1-(3-(2-methyl-4-(4-(trifluoromethyl)phenyl)but-3-en-2-yl)pyrrolidin-1-yl)prop-2-en-1-one as a colorless oil (176 mg, 41% yield). MS (ESI, m/z) calcd. for C₁₉H₂₂F₃NO, 337.16, found, 338.10 [M+H]⁺. ¹H NMR (400 MHz, DMSO-d₆) δ 7.72-7.55 (m, 4H), 6.69-6.42 (m, 3H), 6.10 (dd, J=16.8, 2.4 Hz, 1H), 5.62 (dd, J=10.3, 2.3 Hz, 1H), 3.75-3.33 (m, 2H), 3.29-2.99 (m, 2H), 2.33-2.13 (m, 1H), 1.98-1.82 (m, 1H), 1.79-1.54 (m, 1H), 1.13 (s, 6H); ¹⁹F NMR (376 MHz, DMSO-d₆) δ: −60.78.

### Example 87: Synthesis of (R, Z)-1-(3-(4-(trifluoromethyl)styryl)pyrrolidin-1-yl)prop-2-en-1-one

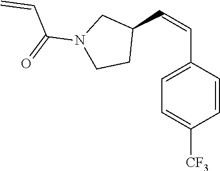 [SMILES: C=CC(=O)N1CC[C@H](/C=C\c2ccc(C(F)(F)F)cc2)C1]

### Synthetic Route

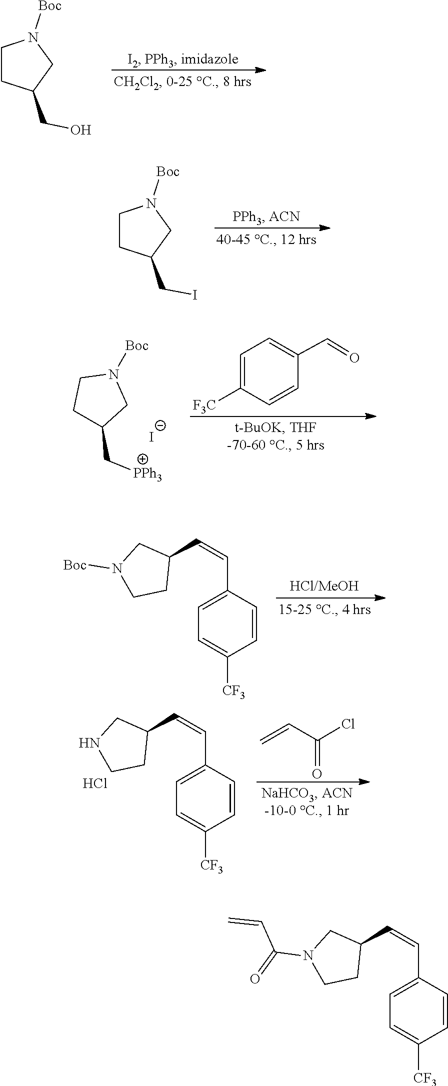

### Synthesis of tert-butyl (S)-3-(iodomethyl)pyrrolidine-1-carboxylate

To the mixed solution of triphenylphosphine (6.52 g, 24.9 mmol) and imidazole (1.69 g, 24.9 mmol) in DCM (17.5 mL) at 0° C. was added I₂ (5.04 g, 19.9 mmol). The suspension was stirred at this temperature for 1 hour. Then tert-butyl (S)-3-(hydroxymethyl)pyrrolidine-1-carboxylate (2 g, 9.94 mmol) was added. The suspension warmed up to room temperature and continued to stir for 8 hours. Then Na₂SO₃ aqueous solution was added. Organic layer was separated, and aqueous layer was extracted with DCM (20 mL×2). Combined organic layer was washed with brine, dried over Na₂SO₄, filtered and concentrated under the reduced pressure. The residue obtained was purified by silica column chromatography (PE/EA=50/1 to 5/1) to afford tert-butyl (S)-3-(iodomethyl) pyrrolidine-1-carboxylate (3.05 g, 98.5% yield) as a colorless oil. MS (ESI, m/z) calcd. for C₁₀H₁₈INO₂, 311.04, found, 255.8 [M-^{t}Bu+1]⁺.

### Synthesis of (S)-((1-(tert-butoxycarbonyl)pyrrolidin-3-yl)methyl)triphenylphosphonium iodide

The suspension solution of tert-butyl (S)-3-(iodomethyl)pyrrolidine-1-carboxylate (3 g, 9.64 mmol) and triphenylphosphine (3.79 g, 14.4 mmol) in acetonitrile (21 mL) was heated between 40 to 45° C. for 12 hours before the reaction was cooled to room temperature. The solvent was removed, then MTBE (about 15 mL) was added and filtered. Solid was further washed with MTBE (5 mL) and dried. (S)-((1-(tert-butoxycarbonyl)pyrrolidin-3-yl)methyl)triphenylphosphonium iodide was obtained (2.0 g, 36.2% yield) as an off-white solid. MS (ESI, m/z) calcd. for C₂₈H₃₃NO₂P⁺, 446.22, found, 446.3 M⁺.

### Synthesis of tert-butyl (R,Z)-3-(4-(trifluoromethyl)styryl)pyrrolidine-1-carboxylate

To a suspension solution of (S)-((1-(tert-butoxycarbonyl)pyrrolidin-3-yl)methyl)triphenyl phosphonium iodide (2 g, 3.49 mmol) in THF (15 mL) between −70 to −60° C. was added potassium tert-butoxide (0.60 g, 5.35 mmol). The solution was stirred at this temperature for 1 hour. Then 4-(trifluoromethyl)benzaldehyde (1.01 g, 5.80 mmol) was added and the reaction solution continued to stir for 3 hours between −70 to −60° C. The solution was warmed up to 0 to 10° C., and ammonium chloride aqueous solution was added. The reaction solution was extracted with ethyl acetate (3×30 mL), washed with brine, dried over Na₂SO₄, filtered and concentrated. The residue obtained was purified by silica column chromatography (PE/EA=50/1 to 5/1) to afford tert-butyl (R,Z)-3-(4-(trifluoromethyl)styryl)pyrrolidine-1-carboxylate (0.8 g, 67.1% yield) as a colorless oil. MS (ESI, m/z) calcd. For C₁₈H₂₂F₃NO₂, 341.16, found, 286.2 [M-^{t}Bu+H]⁺. ¹H NMR (400 MHz, CDCl₃) 7.61 (d, J=8.0 Hz, 2H), 7.34 (d, J=8.0 Hz, 2H), 6.54 (d, J=11.6 Hz, 1H), 5.67 (dd, J=9.6, 11.6 Hz, 1H), 3.59-3.53 (m, 2H), 3.31-3.26 (m, 2H), 3.12-3.07 (m, 1H), 2.06-2.03 (m, 1H), 1.79-1.74 (m, 1H), 1.47 (s, 9H).

### Synthesis of (R,Z)-3-(4-(trifluoromethyl)styryl)pyrrolidine hydrochloride

To a solution of tert-butyl (R, Z)-3-(4-(trifluoromethyl)styryl)pyrrolidine-1-carboxylate (700 mg, 2.05 mmol) in MeOH (5 mL) was added HCl in MeOH (4M, 0.15 mL). The reaction solution was stirred at room temperature for 4 hours. The solvent was removed under the reduced pressure to afford (R,Z)-3-(4-(trifluoromethyl)styryl)pyrrolidine hydrochloride (0.52 g, 91.3% yield) as a yellow oil. MS (ESI, m/z) calcd. for C₁₃H₁₄F₃N 241.11, found, 242.2 [M+H]⁺.

### Synthesis of (R, Z)-1-(3-(4-(trifluoromethyl)styryl)pyrrolidin-1-yl)prop-2-en-1-one

To a solution of (R. Z)-3-(4-(trifluoromethyl)styryl)pyrrolidine hydrochloride (0.40 g, 1.44 mmol) in acetonitrile (5 mL) was added NaHCO₃ (0.278 g, 3.31 mmol). The suspension solution was stirred at 0 to 10° C., and prop-2-enoyl chloride (0.162 g, 1.79 mmol) was added. The reaction solution was stirred at this temperature for 3 hours. The solution was filtered. The cake was washed with ethyl acetate. Filtrate was concentrated under the reduced pressure. Residue obtained was purified by silica column chromatography (PE/EA=50/1 to 5/1) to afford (R, Z)-1-(3-(4-(trifluoromethyl) styryl)pyrrolidin-1-yl)prop-2-en-1-one (0.295 g, 69.4% yield) as a light yellow oil. MS (ESI, m/z) calcd. for C₁₆H₁₆F₃NO 295.1, found, 296.1 [M+H]⁺. ¹H NMR (400 MHz, CDCl₃) δ 7.64-7.60 (m, 2H), 7.35 (d, J=8.0 Hz, 2H), 6.50 (d, J=11.6 Hz, 1H), 6.44-6.39 (m, 2H), 5.71-5.68 (m, 2H), 4.15-3.75 (m, 2H), 3.54-3.51 (m, 1H), 3.33-3.23 (m, 2H), 2.17-2.13 (m, 1H), 1.92-1.65 (m, 1H); ¹⁹F NMR (400 MHz, CDCl₃) δ: −62.50.

### Example 88: Synthesis of (E)-1-(3-(3-(4-(trifluoromethyl)phenyl)prop-1-en-1-yl)pyrrolidin-1-yl)prop-2-en-1-one

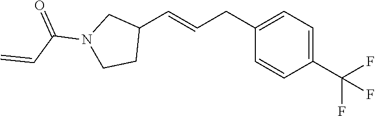 [SMILES: C=CC(=O)N1CCC(/C=C/Cc2ccc(C(F)(F)F)cc2)C1]

### Synthetic Route:

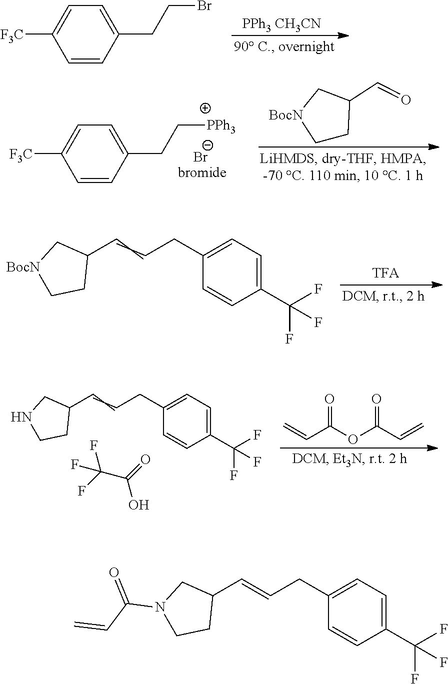

### Synthesis of triphenyl(4-(trifluoromethyl)phenethyl)phosphonium bromide

1-(2-Bromoethyl)-4-(trifluoromethyl)benzene (3 g, 11.9 mmol), a stir bar, triphenylphosphine (3.26 g, 12.4 mmol), ACN (30 mL) were added to a 250 mL round-bottom flask and stirred until homogeneous. The resulting mixture was stirred at 90° C. overnight, then concentrated under the reduced pressure to afford a crude product which was triturated with ethyl ether (50 mL×3) and filtered to afford triphenyl(4-(trifluoromethyl)phenethyl)phosphonium bromide as a white solid (4.27 g, 69.9% yield). MS (ESI, m/z) calcd. for C₂₇H₂₃F₃P⁺, 435.12, found, 435.25 M⁺

### Synthesis of tert-butyl 3-(3-(4-(trifluoromethyl)phenyl)prop-1-en-1-yl)pyrrolidine-1-carboxylate

Triphenyl(4-(trifluoromethyl)phenethyl)phosphonium bromide (4 g, 7.76 mmol), a stir bar and dry-THF (155 mL) were added to an oven-dried and nitrogen-purged 500 mL round-bottom flask and stirred until homogenous, then treated with 1M LiHMDS (7.8 mL, 7.80 mmol) over 10 min at −70° C. under nitrogen atmosphere. After stirring for 40 min, HMPA (12 mL, 68.6 mmol) was added dropwise and stirring continued for 30 min. To the mixture was added a solution of tert-butyl 3-formylpyrrolidine-1-carboxylate (1.87 g, 9.385 mmol) in dry-THF (5 mL) and the resulting mixture was stirred at −70° C. for 30 min, then warmed to 10° C., and stirred for 1 h. The mixture was quenched with NH4Cl solution (40 mL) and extracted with EA (300 mL×3). The combined organic layers were washed with brine, dried over anhydrous sodium sulfate, filtered and concentrated under vacuum. The residue was purified by silica gel chromatography (EA/PE=0-30%) to afford tert-butyl 3-(3-(4-(trifluoromethyl)phenyl)prop-1-en-1-yl)pyrrolidine-1-carboxylate as a yellow oil (500 mg, 18.1% yield). MS (ESI, m/z) calcd. for C₁₉H₂₄F₃NO₂, 355.17, found, 341.05 [M+H-^{t}Bu+ACN]⁺.

### Synthesis of 3-(3-(4-(trifluoromethyl)phenyl)prop-1-en-1-yl)pyrrolidine 2,2,2 trifluoroacetate

tert-Butyl 3-(3-(4-(trifluoromethyl)phenyl)prop-1-en-1-yl)pyrrolidine-1-carboxylate (500 mg, 1.41 mmol), a stir bar, DCM (6 mL) were added to a 20 mL vial and stirred until homogeneous, then treated with TFA (2 mL). The resulting mixture was stirred at room temperature for 2 hours, then concentrated under the reduced pressure to afford 3-(3-(4-(trifluoromethyl)phenyl)prop-1-en-1-yl)pyrrolidine 2,2,2-trifluoroacetate as a yellow oil (550 mg, crude). MS (ESI, m/z) calcd. for C₁₄H₁₆F₃N, 255.12, found, 256.15 [M+H]⁺.

### Synthesis of (E)-1-(3-(3-(4-(trifluoromethyl)phenyl)prop-1-en-1-yl)pyrrolidin-1-yl)prop-2-en-1-one

3-(3-(4-(Trifluoromethyl)phenyl)prop-1-en-1-yl)pyrrolidine 2,2,2-trifluoroacetate (450 mg, 1.22 mmol), a stir bar, DCM (4 mL) were added to a 20 mL vial and stirred until homogeneous before the reaction vessel was cooled to 0° C., then treated with Et₃N (640 mg, 6.33 mmol) and a solution of prop-2-enoyl prop-2-enoate (240 mg, 1.90 mmol) in DCM (1 mL) dropwise. The resulting mixture was stirred at room temperature for 2 hours then quenched with saturated sodium bicarbonate solution (10 mL) and extracted with DCM (50 mL×3). The combined organic extracts were washed with brine, dried over anhydrous Na₂SO₄, filtered and concentrated under vacuum. The residue was purified by reverse-phase chromatography with ACN/H₂O (0.05% NH₄HCO₃) (5%-60%) to afford a crude product, which was further separated by PREP_ACHIRAL_SFC (Column: YMC-Actus Triart Diol-HILIC 3*25 cm, 5 μm; Mobile Phase A: CO₂, Mobile Phase B: IPA:HEX=1:5 (0.1% 2M NH₃-MeOH); Flow rate: 75 mL/min; Gradient: isocratic 20% B; Column Temperature (° C.); 35; Back Pressure (bar); 100; Wave Length: 254 nm; RT1(min): 5.93; RT2(min): 6.89; Sample Solvent: MeOH—HPLC; Injection Volume: 2 mL) to give two products. The second eluting fractions were lyophilized to afford (E)-1-(3-(3-(4-(trifluoromethyl)phenyl)prop-1-en-1-yl)pyrrolidin-1-yl)prop-2-en-1-one as a colorless oil (96.5 mg, 25.6% yield), MS (ESI, m/z) calcd. for C₁₇H₁₈F₃NO, 309.13, found, 310.10 [M+H]⁺. ¹H NMR (400 MHz, DMSO-d₆) δ 7.72-7.57 (m, 4H), 6.64-6.45 (m, 3H), 6.12 (dt, J=16.8, 2.9 Hz, 1H), 5.69-5.59 (m, 1H), 3.82-3.44, 3.08-2.96 (m, 3H), 3.29-3.15 (m, 1H), 2.43-2.22 (m, 3H), 2.15-1.96 (m, 1H), 1.72-1.48 (m, 1H); ¹⁹F NMR (376 MHz, DMSO-d₆) δ: −60.81.

### Example 89: Synthesis of (Z)-1-(3-(3-(4-(trifluoromethyl)phenyl)prop-1-en-1-yl)pyrrolidin-1-yl)prop-2-en-1-one

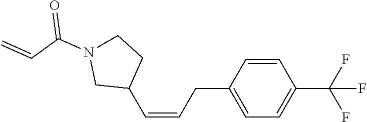 [SMILES: C=CC(=O)N1CCC(/C=C\Cc2ccc(C(F)(F)F)cc2)C1]

### Synthetic Route:

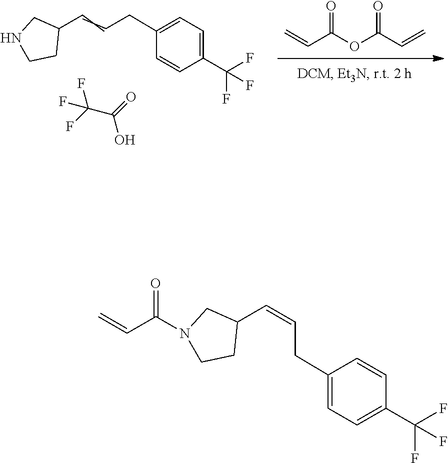

### Synthesis of (Z)-1-(3-(3-(4-(trifluoromethyl)phenyl)prop-1-en-1-yl)pyrrolidin-1-yl)prop-2-en-1-one

3-(3-(4-(Trifluoromethyl)phenyl)prop-1-en-1-yl)pyrrolidine 2,2,2-trifluoroacetate (450 mg, 1.22 mmol), a stir bar, DCM (4 mL) were added to a 20 mL vial and stirred until homogeneous before the reaction vessel was cooled to 0° C., then treated with Et₃N (640 mg, 6.33 mmol) and a solution of prop-2-enoyl prop-2-enoate (240 mg, 1.90 mmol) in DCM (1 mL) dropwise. The resulting mixture was stirred at room temperature for 2 hours then quenched with saturated sodium bicarbonate solution (10 mL) and extracted with DCM (50 mL×3). The combined organic extracts were washed with brine, dried over anhydrous Na₂SO₄, filtered and concentrated under vacuum. The residue was purified by reverse-phase chromatography with ACN/H₂O (0.05% NH₄HCO₃) (5%-60%) to afford a crude product, which was further separated by PREP_ACHIRAL_SFC (Column: YMC-Actus Triart Diol-HILIC 3*25 cm, 5 μm; Mobile Phase A: CO₂, Mobile Phase B: IPA:HEX=1:5 (0.1% 2M NH₃-MeOH); Flow rate: 75 mL/min; Gradient: isocratic 20% B; Column Temperature (° C.); 35; Back Pressure (bar); 100; Wave Length: 254 nm; RT1(min): 5.93; RT2(min): 6.89; Sample Solvent: MeOH—HPLC; Injection Volume: 2 mL) to give two products. The first eluting fractions were lyophilized to afford (Z)-1-(3-(3-(4-(trifluoromethyl)phenyl)prop-1-en-1-yl) pyrrolidin-1-yl)prop-2-en-1-one as a colorless oil (28 mg, 7.5% yield). MS (ESI, m/z) mass calcd. for C₁₇H₁₈F₃NO, 309.13, found, 310.10 [M+H]⁺. ¹H NMR (400 MHz, DMSO-d₆) δ 7.66 (d, J=7.9 Hz, 2H), 7.44 (d, J=8.1, 2.9 Hz, 2H), 6.58 (dd, J=16.8, 10.3, 1.7 Hz, 1H). 6.13 (dt, J=16.8, 2.4 Hz, 1H), 5.69-5.56 (m, 2H), 5.54-5.42 (m, 1H), 3.82-3.69 (m, 1H), 3.66-3.57 (m, 1H), 3.56-3.47 (m, 2H), 3.41-3.32 (m, 1H), 3.28-3.14 (m, 1H), 3.02-2.92 (m, 1H), 2.12-1.93 (m, 1H), 1.79-1.54 (m, 1H); ¹⁹F NMR (376 MHz, DMSO-d₆) δ: −60.71.

### Example 90: Synthesis of (E)-1-(4-(3-(4-(trifluoromethyl)phenyl)allyl)piperidin-1-yl)prop-2-en-1-one

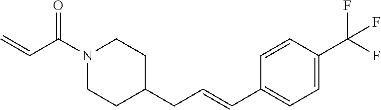 [SMILES: C=CC(=O)N1CCC(C/C=C/c2ccc(C(F)(F)F)cc2)CC1]

### Synthetic Route:

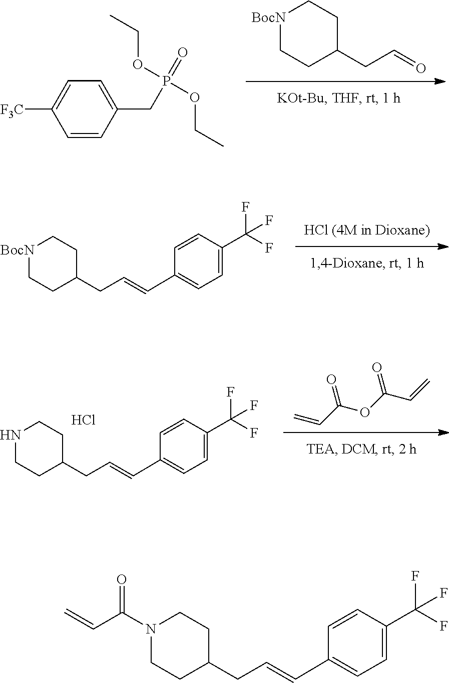

### Synthesis of tert-butyl (E)-4-(3-(4-(trifluoromethyl)phenyl)allyl)piperidine-1-carboxylate

Diethyl (4-(trifluoromethyl)phenyl)methylphosphonate (500 mg, 1.69 mmol), a stir bar, tert-butyl 4-(2-oxoethyl)piperidine-1-carboxylate (422 mg, 1.86 mmol) and THF (13 mL) were added to a 40 mL vial and stirred until homogeneous, then treated with potassium tert-butoxide (227 mg, 2.03 mmol) at 0° C. The resulting mixture was stirred at room temperature for 1 hour, then quenched with H₂O (20 mL) and extracted with EA (50 mL×3). The combined organic layers were washed with brine, dried over anhydrous sodium sulfate, filtered and concentrated. The residue was purified by silica gel chromatography (0-20% EA/PE) to afford tert-butyl (E)-4-(3-(4-(trifluoromethyl)phenyl)allyl)piperidine-1-carboxylate as a white oil (0.523 g, 83.9%). MS (EST, m/z) calcd. for C₂₀H₂₆F₃NO₂, 369.19, found 355.10 [M-^{t}Bu+ACN+H]⁺.

### Synthesis of (E)-4-(3-(4-(trifluoromethyl)phenyl)allyl)piperidine hydrochloride

tert-Butyl (E)-4-(3-(4-(trifluoromethyl)phenyl)allyl)piperidine-1-carboxylate (500 mg, 1.35 mmol), a stir bar and 1,4-dioxane (10 mL) were added to a 100 mL round-bottom flask and stirred until homogenous, then treated with HCl (10 mL, 4M in dioxane). The resulting mixture was stirred at room temperature for 1 hour. The reaction was concentrated under the reduced pressure to afford (E)-4-(3-(4-(trifluoromethyl) phenyl)allyl)piperidine hydrochloride as a white solid (0.47 g, crude). MS (ESI, m/z) calcd. for C₁₅H₁₈F₃N, 269.15, found 270.10 [M+H]⁺.

### Synthesis of (E)-1-(4-(3-(4-(trifluoromethyl)phenyl)allyl)piperidin-1-yl)prop-2-en-1-one

(E)-4-(3-(4-(Trifluoromethyl) phenyl)allyl)piperidine hydrochloride (150 mg, 0.491 mmol), triethylamine (248 mg, 2.46 mmol), a stir bar and DCM (4 mL) were added to a 20 mL vial and stirred until homogenous, then treated with a solution of prop-2-enoyl prop-2-enoate (74 mg, 0.589 mmol) in DCM (1 mL) dropwise at 0° C. The resulting mixture was stirred at room temperature for 2 hours. The reaction solution was quenched with H₂O (10 mL) and extracted with DCM (3×30 mL). The organic layers were combined, dried over anhydrous Na₂SO₄, filtered and concentrated. The residue obtained was purified by Prep-HPLC (Column: XSelect CSH Fluoro Phenyl 30*150 mm, 5 μm; Mobile Phase A: Water (10 mmol/L NH₄HCO₃), Mobile Phase B: ACN; Flow rate: 60 mL/min m/min; Gradient: 17% B to 42% B in 9 min; Wave Length: 254 nm/220 nm nm; RT1(min): 8.8) to afford (E)-1-(4-(3-(4-(trifluoromethyl)phenyl)allyl)piperidin-1-yl)prop-2-en-1-one (56 mg, 35.3%) as a colorless oil. MS (ESI, m/z) calcd. for C₁₈H₂₀F₃NO, 323.14, found 324.15 [M+H]⁺. ¹H NMR (400 MHz, DMSO-d₆) δ 7.74-7.44 (m, 4H), 6.85-6.74 (m, 1H), 6.56-6.42 (m, 2H), 6.07 (dd, J=16.7, 2.5 Hz, 1H), 5.64 (dd, J=10.5, 2.5 Hz, 1H), 4.42 (d, J=13.0 Hz, 1H), 4.05 (d, J=13.6 Hz, 1H), 3.07-2.97 (m, 1H), 2.69-2.57 (m, 1H), 2.23-2.12 (m, 2H), 1.81-1.64 (m, 3H), 1.14-0.97 (m, 2H); ¹⁹F NMR (376 MHz, DMSO-d₆) δ: −60.23.

### Example 91: Synthesis of (7)-1-(3-(3-methyl-3-(4-(trifluoromethyl)phenyl)but-1-en-1-yl) pyrrolidin-1-yl)prop-2-en-1-one

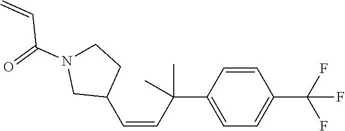 [SMILES: C=CC(=O)N1CCC(/C=C\C(C)(C)c2ccc(C(F)(F)F)cc2)C1]

### Synthetic Route:

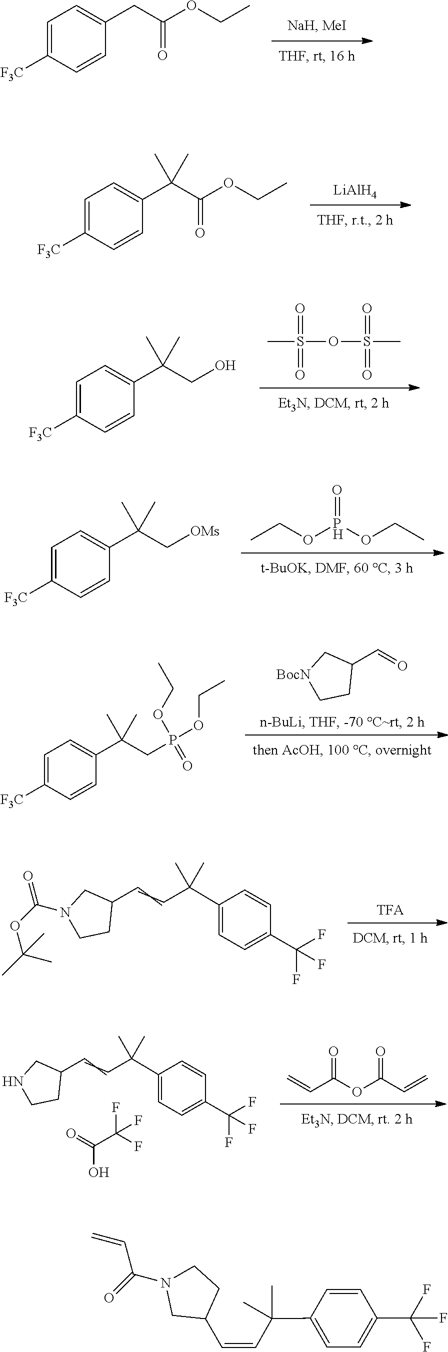

### Synthesis of Ethyl 2-methyl-2-(4-(trifluoromethyl)phenyl) propanoate

Ethyl 2-(4-(trifluoromethyl)phenyl)acetate (10 g, 43.1 mmol), MeI (15.9 g, 112 mmol), a stir bar, and THF (200 mL) were added to a 500 mL round-bottom flask and stirred until homogeneous, and then treated with NaH (4 g, 100 mmol, 60% in oil). The resulting mixture was stirred overnight at room temperature. The reaction mixture was quenched with water (100 mL), extracted with EA (3×200 mL). The combined organic layers were washed with brine, dried over anhydrous Na₂SO₄, filtered, and concentrated under the reduced pressure. The residue was purified by silica gel chromatography (5-10% EA/PE) to afford ethyl 2-methyl-2-(4-(trifluoromethyl)phenyl) propanoate as a colorless oil (5 g, 44.6% yield). MS (ESI, m/z) calcd. for C₁₃H₁₅F₃O₂, 260.10, found 261.00 [M+H]⁺.

### Synthesis of 2-methyl-2-(4-(trifluoromethyl)phenyl)propan-1-ol

Ethyl 2-methyl-2-(4-(trifluoromethyl)phenyl)propanoate (5.2 g, 20.0 mmol), a stir bar, and THF (80 mL) were added to a 250 mL round-bottom flask and stirred until homogeneous, and then treated with LiAlH₄ in THF (1 M, 24 mL, 24.0 mmol) at 0° C. The resulting mixture was stirred for 2 hours at room temperature, then quenched with water (1.8 mL, 99.9 mmol) and anhydrous Na₂SO₄. The mixture was stirred for 20 min, then filtered, washed with THF, and the filtrate was concentrated under the reduced pressure. The residue was purified by silica gel chromatography (30-40% EA/PE) to afford 2-methyl-2-(4-(trifluoromethyl) phenyl)propan-1-ol as colorless oil (4 g, 91.7% yield). ¹H NMR (400 MHz, DMSO-d₆) δ 7.62 (q, J=8.6 Hz, 4H), 4.75 (s, 1H), 3.46 (s, 2H), 1.25 (s, 6H).).

### Synthesis of 2-methyl-2-(4-(trifluoromethyl)phenyl)propyl methanesulfonate

2-Methyl-2-(4-(trifluoromethyl)phenyl)propan-1-ol (4 g, 18.3 mmol), TEA (3.7 g, 36.6 mmol), a stir bar, and DCM (80 mL) were added to a 250 mL round-bottom flask and stirred until homogeneous, and then treated with methanesulfonic anhydride (4.8 g, 27.6 mmol) at 0° C. The resulting mixture was stirred for 2 hours at room temperature. The reaction mixture was then quenched with water (80 mL), extracted with DCM (3×100 mL). The combined organic layers were dried over anhydrous Na₂SO₄, filtered, and concentrated under the reduced pressure. The residue was purified by silica gel chromatography (20-30% EA/PE) to afford 2-methyl-2-(4-(trifluoromethyl)phenyl)propyl methanesulfonate as a white solid (4.7 g, 86.5% yield). ¹H NMR (400 MHz, DMSO-d₆) δ 7.76-7.63 (m, 4H), 4.33 (s, 2H), 3.12 (s, 3H), 1.37 (s, 6H).

### Synthesis of diethyl (2-methyl-2-(4-(trifluoromethyl)phenyl)propyl)phosphonate

Diethyl phosphonate (2.6 g, 18.8 mmol), t-BuOK (2.2 g, 19.6 mmol), a stir bar and DMF (20 mL) were added to a 250 mL round-bottom flask. The resulting mixture was stirred for 30 min at room temperature, then a solution of 2-methyl-2-(4-(trifluoromethyl)phenyl)propyl methanesulfonate (4 g, 13.5 mmol) in DMF (20 mL) was added and stirring continued for 3 h at 60° C. The reaction mixture was then quenched with water (50 mL), extracted with EA (3×100 mL). The combined organic layers were washed with brine (3×30 mL), dried over anhydrous Na₂SO₄, filtered, and concentrated under the reduced pressure. The residue was purified by silica gel chromatography (0-5% MeOH/DCM) to afford diethyl (2-methyl-2-(4-(trifluoromethyl)phenyl)propyl) phosphonate as a white solid (837 mg, 18.3% yield). MS (ESI, m/z) calcd. for C₁₅H₂₂F₃O₃P, 338.13, found 339.10 [M+H]⁺.

### Synthesis of tert-butyl 3-(3-methyl-3-(4-(trifluoromethyl)phenyl)but-1-en-1-yl)pyrrolidine-1-carboxylate

Diethyl 2-methyl-2-[4-(trifluoromethyl)phenyl]propylphosphonate (770 mg, 2.28 mmol), a stir bar, and THF (14 mL) were added to a 100 mL round-bottom flask and stirred until homogeneous, and then treated with n-butyllithium (1 mL, 2.5 M, 2.50 mmol) at −78° C. under nitrogen. The resulting mixture was stirred for 1 hour at −78° C., then treated with a solution of tert-butyl 3-formylpyrrolidine-1-carboxylate (544 mg, 2.73 mmol) in THF (1 mL) at −78° C. The resulting mixture was stirred for 1 hour at room temperature, then treated with acetic acid (15 mL). The resulting mixture was stirred overnight at 100° C., then cooled to room temperature and stirring continued for 2 hours, then quenched with water (100 mL) and extracted with EA (150 mL×3). The combined extracts were dried over anhydrous sodium sulfate, filtered, and concentrated. The residue was purified by reverse-phase chromatography (5%-100% ACN/0.1% FA water) to afford tert-butyl 3-(3-methyl-3-(4-(trifluoromethyl)phenyl)but-1-en-1-yl)pyrrolidine-1-carboxylate as a yellow oil (160 mg, 18.3% yield). MS (ESI, m/z) calcd. for C₂₁H₂₈F₃NO₂, 383.21, found 406.20 [M+Na]⁺.

### Synthesis of 3-(3-methyl-3-(4-(trifluoromethyl)phenyl)but-1-en-1-yl)pyrrolidine 2,2,2-trifluoroacetate

tert-Butyl 3-(3-methyl-3-(4-(trifluoromethyl)phenyl)but-1-en-1-yl)pyrrolidine-1-carboxylate (160 mg, 0.417 mmol), a stir bar and DCM (5 mL) were added to a 20 mL vial and stirred until homogenous, then treated with TFA (1 mL). The resulting mixture was stirred at room temperature for 1 hour and concentrated under the reduced pressure to afford 3-(3-methyl-3-(4-(trifluoromethyl)phenyl)but-1-en-1-yl)pyrrolidine 2,2,2-trifluoroacetate as a red oil (178 mg, crude). MS (ESI, m/z) calcd. for C₁₆H₂₀F₃N, 283.15, found 284.10 [M+H]⁺.

### Synthesis of (Z)-1-(3-(3-methyl-3-(4-(trifluoromethyl)phenyl)but-1-en-1-yl)pyrrolidin-1-yl)prop-2-en-1-one

3-(3-Methyl-3-(4-(trifluoromethyl)phenyl)but-1-en-1-yl)pyrrolidine 2,2,2-trifluoroacetate (170 mg, 0.428 mmol), triethylamine (216 mg, 2.14 mmol), a stir bar and DCM (5 mL) were added to a 20 mL vial and stirred until homogenous, then treated with a solution of prop-2-enoyl prop-2-enoate (64.7 mg, 0.514 mmol) in DCM (1 mL) dropwise at 0° C. The resulting mixture was stirred at room temperature for 2 hours. The reaction solution was quenched with H₂O and extracted with DCM. The organic layers were combined, dried over anhydrous Na₂SO₄, filtered and concentrated. The residue obtained was purified by PREP-ACHIRAL-SFC (Column: GreenSep Naphthyl, 3*25 cm, 5 μm; Mobile Phase A: CO₂, Mobile Phase B: IPA (0.1% 2M NH₃-MEOH); Flow rate: 75 mL/min; Gradient: isocratic 17% B; Column Temperature (° C.); 35; Back Pressure (bar); 100; Wave Length: 254 nm; RT1(min): 7.13; RT2(min): 11.12; Sample Solvent: MeOH—HPLC; Sample concentration: mg/mL; Injection Volume: 3.5 mL) to give two products. The first eluting fractions were lyophilized to afford (Z)-1-(3-(3-methyl-3-(4-(trifluoromethyl)phenyl)but-1-en-1-yl) pyrrolidin-1-yl)prop-2-en-1-one (22 mg, 15.4%) as a yellow oil. MS (ESI, m/z) calcd. for C₁₉H₂₂F₃NO, 337.16, found 338.15 [M+H]⁺. ¹H NMR (400 MHz, DMSO-d₆) δ 7.75-7.49 (m, 4H), 6.51-6.29 (m, 1H), 6.10-5.98 (m, 1H), 5.84-5.76 (m, 1H), 5.67-5.52 (m, 1H), 5.36-5.15 (m, 1H), 3.62-3.43 (m, 1H), 3.24-3.10 (m, 1H), 3.02-2.89 (m, 1H), 2.45-2.30 (m, 1H), 1.61-1.17 (m, 8H), 1.08-0.77 (m, 1H); ¹⁹F NMR (376 MHz, DMSO-d₆) δ: −60.20.

### Example 92: Synthesis of (E)-1-(3-(3-methyl-3-(4-(trifluoromethyl)phenyl)but-1-en-1-yl) pyrrolidin-1-yl)prop-2-en-1-one

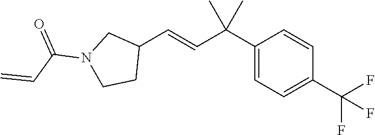 [SMILES: C=CC(=O)N1CCC(/C=C/C(C)(C)c2ccc(C(F)(F)F)cc2)C1]

### Synthetic Route:

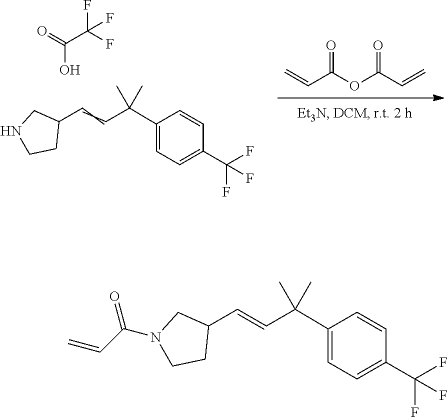

### Synthesis of (E)-1-(3-(3-methyl-3-(4-(trifluoromethyl)phenyl)but-1-en-1-yl) pyrrolidin-1-yl)prop-2-en-1-one

3-(3-Methyl-3-(4-(trifluoromethyl)phenyl)but-1-en-1-yl)pyrrolidine 2,2,2-trifluoroacetate (170 mg, 0.428 mmol), triethylamine (216 mg, 2.14 mmol), a stir bar and DCM (5 mL) were added to a 20 mL vial and stirred until homogenous, then treated with a solution of prop-2-enoyl prop-2-enoate (64.74 mg, 0.514 mmol) in DCM (1 mL) dropwise at 0° C. The resulting mixture was stirred at room temperature for 2 hours. The reaction solution was quenched with H₂O and extracted with DCM. The organic layers were combined, dried over Na₂SO₄, filtered and concentrated. The residue obtained was purified by PREP-ACHIRAL-SFC (Column: GreenSep Naphthyl, 3*25 cm, 5 μm; Mobile Phase A: CO₂, Mobile Phase B: IPA (0.1% 2M NH₃-MEOH); Flow rate: 75 mL/min; Gradient: isocratic 17% B; Column Temperature (° C.); 35; Back Pressure (bar); 100; Wave Length: 254 nm; RT1(min): 7.13; RT2(min): 11.12; Sample Solvent: MeOH—HPLC; Sample concentration: mg/mL; Injection Volume: 3.5 mL) to give two products. The second eluting fractions were lyophilized to afford (E)-1-(3-(3-methyl-3-(4-(trifluoromethyl)phenyl)but-1-en-1-yl) pyrrolidin-1-yl)prop-2-en-1-one (36 mg, 24.9%) as a yellow oil. MS (ESI, m/z) mass calcd. for C₁₉H₂₂F₃NO, 337.16, found 338.15 [M+H]⁺. ¹H NMR (400 MHz, DMSO-d₆) δ 7.73-7.47 (m, 4H), 6.66-6.49 (m, 1H), 6.18-6.04 (m, 1H), 5.83-5.71 (m, 1H), 5.69-5.61 (m, 1H), 5.55-5.42 (m, 1H), 3.81-3.41 (m, 3H), 3.26-3.04 (m, 1H), 2.96-2.77 (m, 1H), 2.13-1.89 (m, 1H), 1.83-1.57 (m, 1H), 1.43-1.19 (m, 6H); ¹⁹F NMR (376 MHz, DMSO-d₆) δ: −60.20.

### Example 93: Synthesis of (S, E)-1-(3-(3-(4-(trifluoromethyl)phenyl)prop-1-en-1-yl) pyrrolidin-1-yl)prop-2-en-1-one

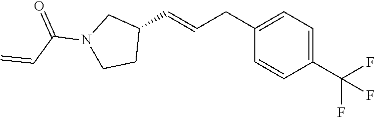 [SMILES: C=CC(=O)N1CC[C@@H](/C=C/Cc2ccc(C(F)(F)F)cc2)C1]

### Synthetic Route:

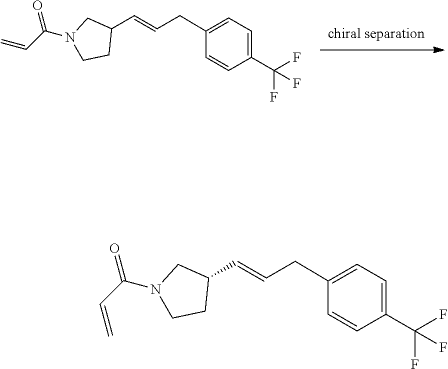 [SMILES: C=CC(=O)N1CCC(/C=C/Cc2ccc(C(F)(F)F)cc2)C1 ; C=CC(=O)N1CC[C@@H](/C=C/Cc2ccc(C(F)(F)F)cc2)C1]

### Synthesis of (S, E)-1-(3-(3-(4-(trifluoromethyl)phenyl)prop-1-en-1-yl) pyrrolidin-1-yl)prop-2-en-1-one

(E)-1-(3-(3-(4-(Trifluoromethyl)phenyl)prop-1-en-1-yl)pyrrolidin-1-yl)prop-2-en-1-one (30 mg, 0.10 mmol) was separated by Chiral HPLC (Column: CHIRALPAK AS 2*25 cm, 5 μm; Mobile Phase A: HEX (0.5% 2M NH₃-MeOH), Mobile Phase B: ETOH; Flow rate: 20 mL/min; Gradient: isocratic 10; Wave Length: 220/254 nm; RT1(min): 17.549; RT2(min): 20.272). The first eluting fractions were lyophilized to afford (S, E)-1-(3-(3-(4-(trifluoromethyl)phenyl)prop-1-en-1-yl) pyrrolidin-1-yl)prop-2-en-1-one (7.2 mg, 24% yield) as a colorless oil. MS (ESI, m/z) calcd. for C₁₇H₁₈F₃NO 309.13, found 310.15[M+H]⁺. ¹H NMR (400 MHz, DMSO-d₆) δ 7.70-7.61 (m, 4H), 6.63-6.45 (m, 3H), 6.15-6.10 (m, 1H), 5.67-5.62 (m, 1H), 3.80-3.45 (m, 2H), 3.32-2.97 (m, 2H), 2.43-2.23 (m, 3H), 2.13-1.92 (m, 1H), 1.74-1.45 (m, 1H).

### Example 94: Synthesis of (R, E)-1-(3-(3-(4-(trifluoromethyl)phenyl)prop-1-en-1-yl) pyrrolidin-1-yl)prop-2-en-1-one

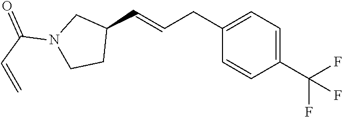 [SMILES: C=CC(=O)N1CC[C@H](/C=C/Cc2ccc(C(F)(F)F)cc2)C1]

### Synthetic Route:

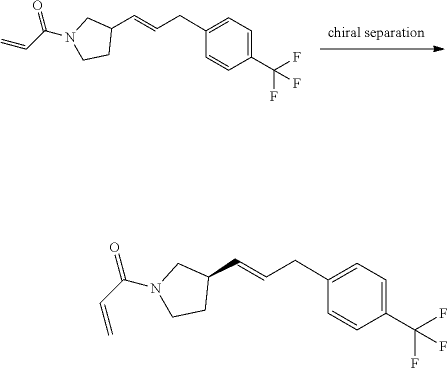 [SMILES: C=CC(=O)N1CCC(/C=C/Cc2ccc(C(F)(F)F)cc2)C1 ; C=CC(=O)N1CC[C@H](/C=C/Cc2ccc(C(F)(F)F)cc2)C1]

### Synthesis of (R, E)-1-(3-(3-(4-(trifluoromethyl)phenyl)prop-1-en-1-yl) pyrrolidin-1-yl)prop-2-en-1-one

(E)-1-(3-(3-(4-(trifluoromethyl)phenyl)prop-1-en-1-yl)pyrrolidin-1-yl)prop-2-en-1-one (30 mg, 0.10 mmol) was separated by Chiral HPLC (Column: CHIRALPAK AS 2*25 cm, 5 μm; Mobile Phase A: HEX (0.5% 2M NH₃-MeOH), Mobile Phase B: ETOH; Flow rate: 20 mL/min; Gradient: isocratic 10; Wave Length: 220/254 nm; RT1(min): 17.549; RT2(min): 20.272). The second eluting fractions were lyophilized to afford (R, E)-1-(3-(3-(4-(trifluoromethyl)phenyl)prop-1-en-1-yl) pyrrolidin-1-yl)prop-2-en-1-one (7.2 mg, 24% yield) as a colorless oil. MS (ESI, m/z) calcd. for C₁₇H₁₈F₃NO 309.13, found 310.15[M+H]⁺. ¹H NMR (400 MHz, DMSO-d₆) δ 7.70-7.60 (m, 4H), 6.63-6.45 (m, 3H), 6.15-6.09 (m, 1H), 5.67-5.62 (m, 1H), 3.80-3.45 (m, 2H), 3.34-3.15 (m, 1H), 3.06-3.01 (m, 1H), 2.45-2.23 (m, 3H), 2.14-1.97 (m, 1H), 1.67-1.51 (m, 1H).

### Example 95: Synthesis of 1-(7-(4-(trifluoromethyl)phenyl)-2-azaspiro[4.4]non-6-en-2-yl)prop-2-en-1-one

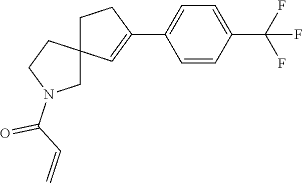 [SMILES: C=CC(=O)N1CCC2(C=C(c3ccc(C(F)(F)F)cc3)CC2)C1]

### Synthetic Route:

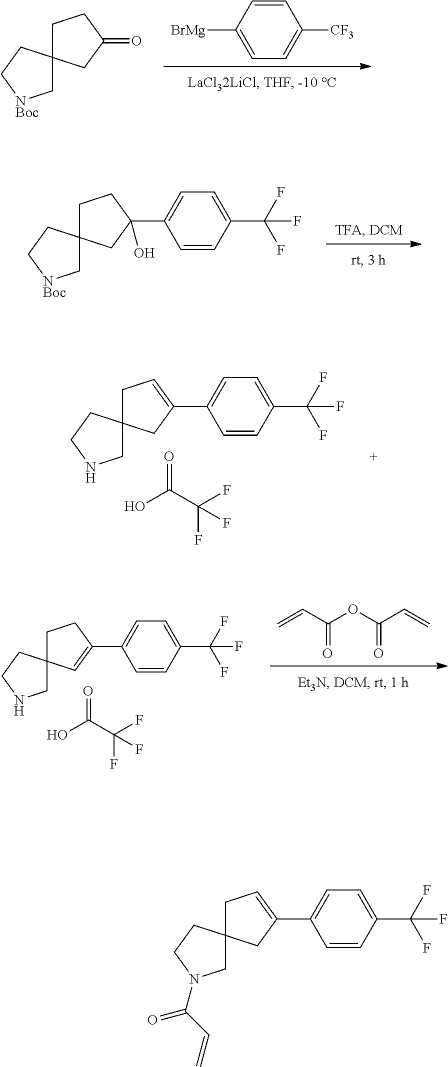

### Synthesis of tert-butyl 7-hydroxy-7-(4-(trifluoromethyl)phenyl)-2-azaspiro[4.4]nonane-2-carboxylate

tert-Butyl 7-oxo-2-azaspiro[4.4]nonane-2-carboxylate (1.63 g, 6.81 mmol), a stir bar and THF (20 mL) were added to a 250 mL three-necked flask and stirred until homogeneous, then treated with LaCl₃·2LiCl (34 mL, 20.4 mmol, 0.6 M in THF) dropwise at −10° C. under nitrogen atmosphere. The resulting mixture was stirred for 30 min at −10° C. under nitrogen atmosphere, then (4-(trifluoromethyl)phenyl)magnesium bromide (34 mL, 20.4 mmol, 0.6 M in Et₂O) was added dropwise under nitrogen atmosphere in the ice bath. Then the resulting mixture was continued stirring for 1 h in the ice bath, quenched with saturated NH₄Cl (aq.), extracted with DCM (100 mL×3). The combined organic layers were washed with brine, dried over Na₂SO₄ and concentrated under the reduced pressure. The obtained residue was purified by reverse phase chromatography (5%-60% ACN/10 mmM NH₄HCO₃ aqueous solution) to afford tert-butyl 7-hydroxy-7-[4-(trifluoromethyl)phenyl]-2-azaspiro[4.4]nonane-2-carboxylate (2.0 g, 76.2% yield) as a white solid. MS (ESI, m/z) calcd. for C₂₀H₂₆F₃NO₃ 385.18, found 353.10[M-^{t}Bu+Na]⁺.

### Synthesis of 7-(4-(trifluoromethyl)phenyl)-2-azaspiro[4.4]non-7-ene 2,2,2-trifluoroacetate and 7-(4-(trifluoromethyl)phenyl)-2-azaspiro[4.4]non-6-ene 2,2,2-trifluoroacetate

tert-Butyl 7-hydroxy-7-[4-(trifluoromethyl)phenyl]-2-azaspiro[4.4]nonane-2-carboxylate (390 mg, 1.01 mmol), a stir bar and DCM (8 mL) were added to a 20 mL vial and stirred until homogeneous, then treated with TFA (0.8 mL) dropwise at 0° C. The resulting mixture was stirred for 3 h at 0° C., concentrated under the reduced pressure to afford a mixture of 7-(4-(trifluoromethyl)phenyl)-2-azaspiro[4.4]non-7-ene 2,2,2-trifluoroacetate and 7-(4-(trifluoromethyl)phenyl)-2-azaspiro[4.4]non-6-ene 2,2,2-trifluoroacetate (700 mg, crude) as a light yellow oil. MS (ESI, m/z) calcd. for C₁₅H₁₆F₃N 267.12, found 268.15 [M+H]⁺.

### Synthesis of 1-(7-(4-(trifluoromethyl)phenyl)-2-azaspiro[4.4]non-6-en-2-yl)prop-2-en-1-one

A mixture of 7-(4-(trifluoromethyl)phenyl)-2-azaspiro[4.4]non-7-ene 2,2,2-trifluoroacetate and 7-(4-(trifluoromethyl)phenyl)-2-azaspiro[4.4]non-6-ene 2,2,2-trifluoroacetate (700 mg, 1.74 mmol), a stir bar, DCM (6.5 mL) and Et₃N (556 mg, 5.49 mmol) were added to a 40 mL vial and stirred until homogeneous, then treated with a solution of prop-2-enoyl prop-2-enoate (348 mg, 2.76 mmol) in DCM (1.5 mL) dropwise at 0° C. The resulting mixture was stirred for 1 h at 0° C., The resulting mixture was extracted with DCM (20 mL×3), the combined organic layers were washed with brine, dried over anhydrous Na₂SO₄ and concentrated. The obtained residue was purified by silica gel column (0%~80% EA in PE) to afford a mixture of 1-(7-(4-(trifluoromethyl)phenyl)-2-azaspiro[4.4]non-7-en-2-yl)prop-2-en-1-one and 1-(7-([4-(trifluoromethyl)phenyl)-2-azaspiro[4.4]non-6-en-2-yl)prop-2-en-1-one (250 mg). The mixture was separated by Prep-HPLC (Column: XSelect CSH C18 Column, 19*250 mm, 5 μm; Mobile Phase A: Water (0.1% FA), Mobile Phase B: ACN; Flow rate: 25 mL/min mL/min; Gradient: 33% B to 58% B in 15 min; Wave Length: 254 nm nm; RT1(min): 14.3, 14.59(min)). The second eluting fractions were lyophilized to afford 1-(7-(4-(trifluoromethyl)phenyl)-2-azaspiro[4.4]non-6-en-2-yl)prop-2-en-1-one (8.3 mg, 1.4% yield) as a white solid. MS (ESI, m/z) calcd. for C₁₈H₁₈F₃NO, 321.13, found, 322.10[M+H]⁺. ¹H NMR (400 MHz, DMSO-d₆) δ 7.71-7.69 (m, 4H), 6.65-6.53 (m, 1H), 6.46-6.39 (m, 1H), 6.18-6.12 (m, 1H), 5.70-5.64 (m, 1H), 3.81-3.34 (m, 4H), 2.85-2.76 (m, 2H), 2.10-1.77 (m, 4H).

### Example 96: Synthesis of 1-(7-(4-(trifluoromethyl)phenyl)-2-azaspiro[4.4]non-7-en-2-yl) prop-2-en-1-one

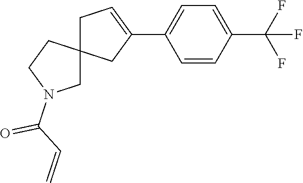 [SMILES: C=CC(=O)N1CCC2(CC=C(c3ccc(C(F)(F)F)cc3)C2)C1]

### Synthetic Route:

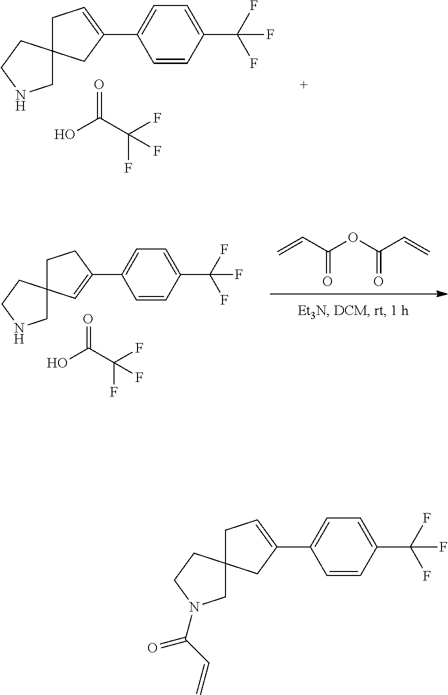

### Synthesis of 1-(7-(4-(trifluoromethyl)phenyl)-2-azaspiro[4.4]non-7-en-2-yl)prop-2-en-1-one

A mixture of 7-(4-(trifluoromethyl)phenyl)-2-azaspiro[4.4]non-7-ene 2,2,2-trifluoroacetate and 7-(4-(trifluoromethyl)phenyl)-2-azaspiro[4.4]non-6-ene 2,2,2-trifluoroacetate (700 mg, 1.74 mmol), a stir bar, DCM (6.5 mL) and Et₃N (556 mg, 5.49 mmol) were added to a 40 mL vial and stirred until homogeneous, then treated with a solution of prop-2-enoyl prop-2-enoate (348 mg, 2.76 mmol) in DCM (1.5 mL) dropwise at 0° C. The resulting mixture was stirred for 1 h at 0° C. The resulting mixture was extracted with DCM (20 mL×3), the combined organic layers were washed with brine, dried over Na₂SO₄ and concentrated under the reduced pressure. The residue obtained was purified by silica gel column (0%~80% EA in PE) to afford a mixture of 1-(7-(4-(trifluoromethyl)phenyl]-2-azaspiro[4.4]non-7-en-2-yl)prop-2-en-1-one and 1-(7-(4-(trifluoromethyl)phenyl]-2-azaspiro[4.4]non-6-en-2-yl)prop-2-en-1-one (250 mg). The mixture was separated by Prep-HPLC (Column: XSelect CSH C18 Column, 19*250 mm, 5 μm; Mobile Phase A: Water (0.1% FA). Mobile Phase B: ACN; Flow rate: 25 mL/min mL/min; Gradient: 33% B to 58% B in 15 min; Wave Length: 254 nm nm; RT1(min): 14.3, 14.59(min)). The first eluting fractions were lyophilized to afford 1-(7-(4-(trifluoromethyl)phenyl)-2-azaspiro[4.4]non-7-en-2-yl)prop-2-en-1-one (36.8 mg, 6.2% yield) as a white solid, MS (ESI, m/z) calcd. for C₁₈H₁₈F₃NO, 321.13, found, 322.05 [M+H]⁺. ¹H NMR (400 MHz, DMSO-d₆) δ 7.70-7.66 (m, 4H), 6.64-6.51 (m, 1H), 6.47-6.44 (m, 1H), 6.18-6.11 (m, 1H), 5.70-5.62 (m, 1H), 3.68-3.64 (m, 1H), 3.61-3.47 (m, 2H), 3.41 (q, J=11.8 Hz, 1H), 2.77-2.72 (m, 2H), 2.58-2.54 (m, 2H), 1.93-1.84 (m, 2H).

### Example 97: Synthesis of 1-(3-(4-(trifluoromethyl)phenyl)-1-oxa-7-azaspiro[4.4]non-3-en-7-yl)prop-2-en-1-one

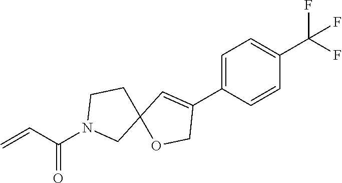 [SMILES: C=CC(=O)N1CCC2(C=C(c3ccc(C(F)(F)F)cc3)CO2)C1]

### Synthetic Route:

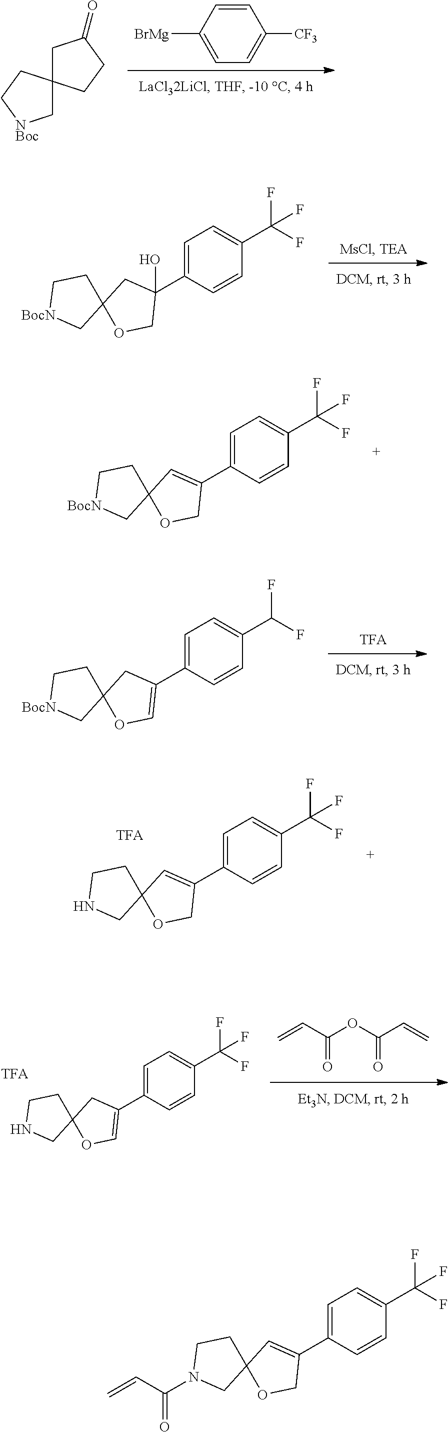

### Synthesis of tert-butyl 3-hydroxy-3-(4-(trifluoromethyl)phenyl)-1-oxa-7-azaspiro[4.4]nonane-7-carboxylate

tert-Butyl 3-oxo-1-oxa-7-azaspiro[4.4]nonane-7-carboxylate (1.5 g, 6.22 mmol), a stir bar, THF (10 mL) and 0.6 M bromo[4-(trifluoromethyl)phenyl]magnesium in THF (15.56 mL, 9.336 mmol) were added to 40 mL vial and stirred until homogenous, then treated with LaCl₃·2LiCl in THF (15.6 mL, 0.6M. 9.34 mmol) dropwise at −10° C. The resulting mixture was stirred for 4 h at −10° C., then quenched with water. The resulting mixture was extracted with DCM (40 mL×3). The combined organic layers were washed with brine, dried over anhydrous sodium sulfate, filtered and concentrated. The residue was purified by silica gel chromatography (0-80% EA in PE) to afford tert-butyl 3-hydroxy-3-(4-(trifluoromethyl) phenyl)-1-oxa-7-azaspiro[4.4]nonane-7-carboxylate (1.9 g, 78.9% yield) as a yellow oil. MS (ESI, m/z) calcd. for C₁₉H₂₄F₃NO₄, 387.00, found, 373.10[M-tBu+ACN+H]⁺.

### Synthesis of tert-butyl 3-(4-(trifluoromethyl)phenyl)-1-oxa-7-azaspiro[4.4]non-3-ene-7-carboxylate and tert-butyl 3-(4-(trifluoromethyl)phenyl)-1-oxa-7-azaspiro[4.4]non-2-ene-7-carboxylate

tert-Butyl 3-hydroxy-3-(4-(trifluoromethyl) phenyl)-1-oxa-7-azaspiro[4.4]nonane-7-carboxylate (1 g, 2.58 mmol), a stir bar, DCM (10 mL) and TEA (0.79 g, 7.81 mmol) were added to 40 mL vial and stirred until homogenous, then treated with MsCl (1.04 g, 9.08 mmol) dropwise at room temperature. The resulting mixture was stirred for 3 hours at room temperature, then quenched with water. The resulting mixture was extracted with DCM (10 mL×3). The combined organic layers were washed with brine, dried over anhydrous sodium sulfate, filtered and concentrated to afford a mixture of tert-butyl 3-(4-(trifluoromethyl) phenyl)-1-oxa-7-azaspiro[4.4]non-3-ene-7-carboxylate and tert-butyl 3-(4-(trifluoromethyl)phenyl)-1-oxa-7-azaspiro[4.4]non-2-ene-7-carboxylate (1.6 g, crude) as a yellow oil. MS (ESI, m/z) calcd. for C₁₉H₂₂F₃NO₃ 369.00, found 314.00[M-tBu+H]⁺.

### Synthesis of 3-(4-(trifluoromethyl)phenyl)-1-oxa-7-azaspiro[4.4]non-3-ene 2,2,2-trifluoroacetate and 3-(4-(trifluoromethyl)phenyl)-1-oxa-7-azaspiro[4.4]non-2-ene trifluoroacetate

A mixture of tert-butyl 3-(4-(trifluoromethyl) phenyl)-1-oxa-7-azaspiro[4.4]non-3-ene-7-carboxylate and tert-butyl 3-(4-(trifluoromethyl)phenyl)-1-oxa-7-azaspiro[4.4]non-2-ene-7-carboxylate (1.6 g, crude mmol), a stir bar and DCM (18 mL) were added to 40 mL vial and stirred until homogenous, then treated with trifluoroacetic acid (6 mL, 26.310 mmol) dropwise at 25° C. The resulting mixture was stirred for 3 h at 25° C., then concentrated to afford a mixture of 3-(4-(trifluoromethyl)phenyl)-1-oxa-7-azaspiro[4.4]non-3-ene 2,2,2-trifluoroacetate and 3-(4-(trifluoromethyl)phenyl)-1-oxa-7-azaspiro[4.4]non-2-ene 2,2,2-trifluoroacetate (3.3 g, crude) as a yellow oil. MS (ESI, m/z) calcd. for C₁₆H₁₅F₅NO₃, 269.00, found, 270.10[M+H]⁺.

### Synthesis of 1-(3-(4-(trifluoromethyl)phenyl)-1-oxa-7-azaspiro[4.4]non-3-en-7-yl)prop-2-en-1-one

A mixture of 3-(4-(trifluoromethyl)phenyl)-1-oxa-7-azaspiro[4.4]non-3-ene 2,2,2-trifluoroacetate and 3-(4-(trifluoromethyl)phenyl)-1-oxa-7-azaspiro[4.4]non-2-ene 2,2,2-trifluoroacetate (600 mg, 1.57 mmol), a stir bar, DCM (9 mL) and TEA (230 mg, 2.27 mmol) were added to a 20 mL vial and stirred until homogenous, then treated with a solution of prop-2-enoyl prop-2-enoate (180 mg, 1.43 mmol) in DCM (1 mL) at room temperature. The resulting mixture was stirred for 2 hours at room temperature, then quenched with water. The resulting mixture was extracted with DCM (10 mL×3). The combined organic layers were washed with brine, dried over anhydrous sodium sulfate, filtered and concentrated. The residue was further purified by silica gel chromatography (0-100% EA in PE) to afford a crude product, which was further separated by Prep-Chiral-HPLC with (Column: Xselect CSH Prep Fluoro-Phenyl Column, 30*150 mm, 5 μm; Mobile Phase A: Water (10 mmol/L NH₄HCO₃). Mobile Phase B: MeOH; Flow rate: 60 mL/min mL/min; Gradient: 40% B to 70% B in 10 min; Wave Length: 254 nm/220 nm nm; RT1(min): 9.02; RT2(min): 9.87) to give two products. The first eluting fractions were lyophilized to afford 1-(3-(4-(trifluoromethyl)phenyl)-1-oxa-7-azaspiro[4.4]non-3-en-7-ylprop-2-en-1-one (30.8 mg, 6.0% yield) as a colorless oil. MS (ESI, m/z) calcd. for C₁₇H₁₆F₃NO₂, 323.00, found, 324.05[M+H]⁺. ¹H NMR (400 MHz, DMSO-d₆) δ 7.75 (d, J=8.1 Hz, 2H), 7.69-7.62 (m, 2H), 6.69-6.49 (m, 2H), 6.21-6.11 (m, 1H), 5.74-5.62 (m, 1H), 5.05-4.93 (m, 2H), 3.88-3.39 (m, 4H), 2.27-1.93 (m, 2H); ¹⁹F NMR (376 MHz, DMSO-d₆) δ, −61.05, −61.06 (d, J=3.2 Hz).

### Example 98: Synthesis of 1-(3-(4-(trifluoromethyl)phenyl)-1-oxa-7-azaspiro[4.4]non-2-en-7-yl)prop-2-en-1-one

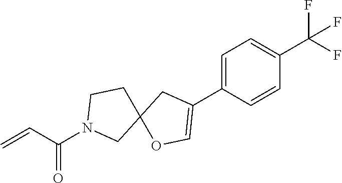 [SMILES: C=CC(=O)N1CCC2(CC(c3ccc(C(F)(F)F)cc3)=CO2)C1]

### Synthetic Route:

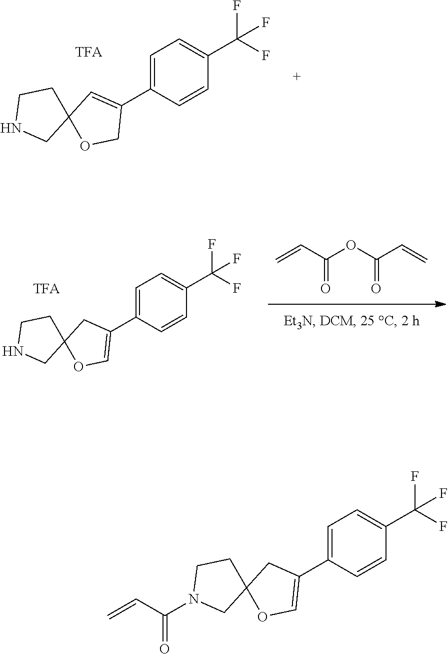

### Synthesis of 1-(3-(4-(trifluoromethyl)phenyl)-1-oxa-7-azaspiro[4.4]non-2-en-7-yl)prop-2-en-1-one

A mixture of 3-(4-(trifluoromethyl)phenyl)-1-oxa-7-azaspiro[4.4]non-3-ene 2,2,2-trifluoroacetate and 3-(4-(trifluoromethyl)phenyl)-1-oxa-7-azaspiro[4.4]non-2-ene 2,2,2-trifluoroacetate (600 mg, 1.57 mmol), a stir bar, DCM (9 mL) and TEA (230 mg, 2.27 mmol) were added to a 20 mL vial and stirred until homogenous, then treated with a solution of prop-2-enoyl prop-2-enoate (180 mg, 1.43 mmol) in DCM (1 mL) at room temperature. The resulting mixture was stirred for 2 hours at room temperature, then quenched with water. The resulting mixture was extracted with DCM (10 mL×3). The combined organic layers were washed with brine, dried over anhydrous sodium sulfate, filtered and concentrated. The residue was further purified by silica gel chromatography (0-100% EA in PE) to afford a crude product, which was further separated by Prep-Chiral-HPLC with (Column: Xselect CSH Prep Fluoro-Phenyl Column, 30*150 mm, 5 μm; Mobile Phase A: Water (10 mmol/L NH₄HCO₃), Mobile Phase B: MeOH; Flow rate: 60 mL/min mL/min; Gradient: 40% B to 70% B in 10 min; Wave Length: 254 nm/220 nm nm; RT1(min): 9.02; RT2(min): 9.87) to give two products. The second eluting fractions were lyophilized to afford 1-(3-(4-(trifluoromethyl)phenyl)-1-oxa-7-azaspiro[4.4]non-2-en-7-yl)prop-2-en-1-one (5.0 mg, 0.94% yield) as a colorless oil. MS (ESI, m/z) calcd. for C₁₇H₁₆F₃NO₂, 323.00, found, 374.05 [M+H]⁺. ¹H NMR (400 MHz, DMSO-d₆) δ 7.63 (d, J=8.2 Hz, 2H), 7.49 (d, J=8.1 Hz, 2H), 7.39 (d, J=8.2 Hz, 1H), 6.68-6.49 (m, 1H), 6.22-6.11 (m, 1H), 5.75-5.63 (m, 1H), 3.95-3.59 (m, 3H), 3.51-3.41 (m, 1H), 3.15-2.87 (m, 2H), 2.34-2.01 (m, 2H); 1⁹F NMR (376 MHz, DMSO-d₆) δ −60.60.

### Example 99: Synthesis of (E)-1-(3-(3-hydroxy-3-(4-(trifluoromethyl)phenyl)prop-1-en-1-yl)pyrrolidin-1-yl)prop-2-en-1-one

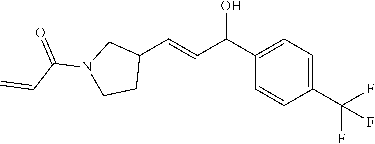 [SMILES: C=CC(=O)N1CCC(/C=C/C(O)c2ccc(C(F)(F)F)cc2)C1]

### Synthetic Route:

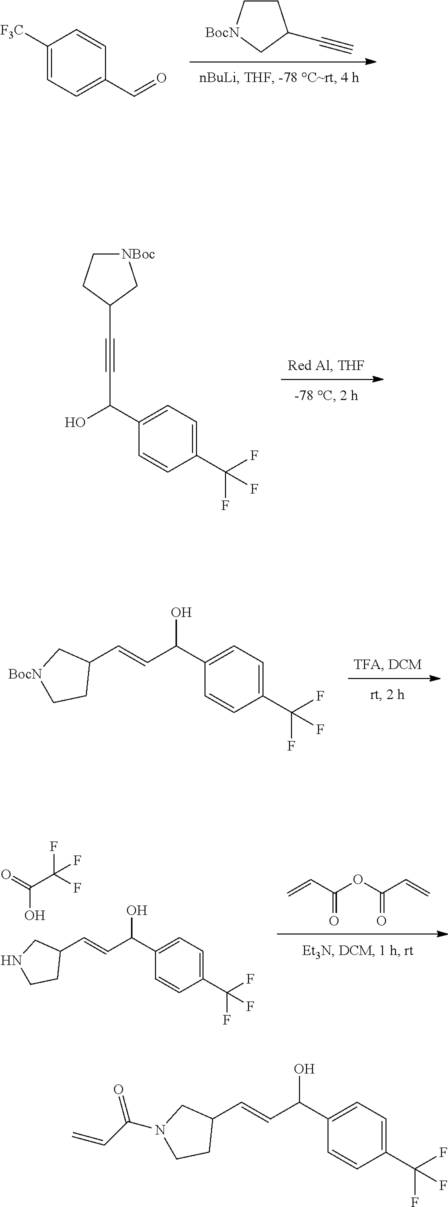

### Synthesis of tert-butyl 3-(3-hydroxy-3-(4-(trifluoromethyl)phenyl)prop-1-yn-1-yl) pyrrolidine-1-carboxylate

tert-Butyl 3-ethynylpyrrolidine-1-carboxylate (3 g, 15.4 mmol), a stir bar and THF (37 mL) were added to a 100 mL three-neck flask and stirred until homogeneous, then cooled to −78° C., treated with butyllithium (7.5 mL, 18.8 mmol, 2.5M) dropwise at −78° C. The resulting mixture was stirred for 2 hours and warmed up to −10° C. slowly, then cooled to −78° C. The reaction solution was treated with 4-(trifluoromethyl) benzaldehyde (3.21 g, 18.4 mmol) in THF (3 mL) dropwise at −78° C. The reaction continued to stir for 2 hours at room temperature, then was quenched with saturated NH₄Cl aqueous solution (30 mL) and extracted with DCM (50 mL×3). The combined organic layers were washed with brine, dried over anhydrous sodium sulfate, filtered and concentrated. The residue was purified by silica gel chromatography (0%-40% EA in PE) to give tert-butyl 3-(3-hydroxy-3-(4-(trifluoromethyl)phenyl)prop-1-yn-1-yl)pyrrolidine-1-carboxylate (3.2 g, 56.4%) as a colorless oil. MS (ESI, m/z) calcd. for C₁₉H₂₂F₃NO₃, 369.16, found, 392.15 [M+Na]⁺.

### Synthesis of tert-butyl (E)-3-(3-hydroxy-3-(4-(trifluoromethyl)phenyl)prop-1-en-1-yl)pyrrolidine-1-carboxylate

tert-Butyl 3-(3-hydroxy-3-(4-(trifluoromethyl)phenyl)prop-1-yn-1-yl)pyrrolidine-1-carboxylate (500 mg, 1.35 mmol), a stir bar and THF (10 mL) were added to a 40 mL vial and stirred until homogeneous, then cooled to −78° C., and treated with Red-Al (4 mL, 3.5M, 14.0 mmol) dropwise at −78° C. The resulting mixture was stirred for 1 hour at −78° C., then quenched with saturated potassium sodium tartrate solution and exacted with DCM (30 mL×3). The combined organic layers were washed with brine, dried over anhydrous sodium sulfate, filtered and concentrated. The residue obtained was purified by silica gel chromatography (0%-38% EA in PE) to give tert-butyl (E)-3-(3-hydroxy-3-(4-(trifluoromethyl)phenyl) prop-1-en-1-yl)pyrrolidine-1-carboxylate (370 mg, 73.6%) as a yellow oil. MS (ESI, m/z) calcd. for C₁₉H₂₄F₃NO₃, 371.17, found, 326.10 [M-^{t}Bu+H]⁺.

### Synthesis of (E)-3-(pyrrolidin-3-yl)-1-(4-(trifluoromethyl)phenyl)prop-2-en-1-ol 2,2,2-trifluoroacetate

tert-Butyl (E)-3-(3-hydroxy-3-(4-(trifluoromethyl)phenyl) prop-1-en-1-yl)pyrrolidine-1-carboxylate (150 mg, 0.404 mmol), a stir bar, DCM (3.5 mL) were added to a 25 mL round bottom flask and stirred until homogeneous, then treated with TFA (0.7 mL) dropwise at room temperature. The resulting mixture was stirred for 2 hours at room temperature and concentrated under the reduced pressure to give (E)-3-(pyrrolidin-3-yl)-1-(4-(trifluoromethyl)phenyl)prop-2-en-1-ol 2,2,2-trifluoroacetate (220 mg, crude) as a brown oil. MS (ESI, m/z) calcd. for C₁₄H₁₆F₃NO, 271.12, found, 270.10 [M−H]⁺.

### Synthesis of (E)-1-(3-(3-hydroxy-3-(4-(trifluoromethyl)phenyl)prop-1-en-1-yl)pyrrolidin-1-yl)prop-2-en-1-one

(E)-3-(pyrrolidin-3-yl)-1-(4-(trifluoromethyl)phenyl)prop-2-en-1-ol 2,2,2-trifluoroacetate (200 mg, 0.519 mmol), a stir bar, Et₃N (180 mg, 1.78 mmol) and DCM (3.0 mL) were added to a 25 mL round bottom flask and stirred until homogeneous, then treated with a solution of prop-2-enoyl prop-2-enoate (55 mg, 0.436 mmol) in DCM (0.5 mL) dropwise at 0° C. The resulting mixture was stirred for 1 hour at room temperature, diluted with water (5 mL) and extracted with DCM (10 mL×3). The combined organic layers were washed with brine, dried over anhydrous sodium sulfate, filtered and concentrated. The residue obtained was purified by PREP-HPLC (Column. XBridge Prep Pheny OBD Column, 19*250 mm, 5 μm; Mobile Phase A: Water (10 mmol/L NH₄HCO₃), Mobile Phase B: ACN; Flow rate: 25 mL/min mL/min; Gradient: 33% B to 63% B in 7 min; Wave Length: 254 nm nm; RT1(min): 6.28) to give (E)-1-(3-(3-hydroxy-3-(4-(trifluoromethyl)phenyl)prop-1-en-1-yl)pyrrolidin-1-yl)prop-2-en-1-one as a yellow semi-solid (11 mg, 6.1% yield). MS (ESI, m/z) calcd. for C₁₇H₁₈F₃NO₂, 325.13, found, 326.10 [M+H]⁺. ¹H NMR (400 MHz, DMSO-d₆) δ 7.67 (s, 4H), 6.73-6.63 (m, 1H), 6.63-6.44 (m, 2H), 6.15-6.06 (m, 1H), 5.68-5.58 (m, 1H), 5.32-5.22 (m, 1H), 4.13-4.08 (m, 1H), 3.74-3.60 (m, 1H), 3.59-3.39 (m, 2H), 3.29-3.11 (m, 1H), 2.42-2.21 (m, 1H), 2.08-1.63 (m, 2H); 19F NMR (376 MHz, DMSO-d₆) δ −60.84.

### Example 100: Synthesis of (Z)-1-(3-(3-hydroxy-3-(4-(trifluoromethyl)phenyl)prop-1-en-1-yl)pyrrolidin-1-yl)prop-2-en-1-one

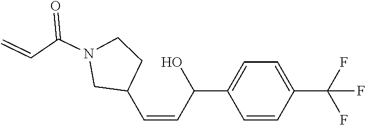 [SMILES: C=CC(=O)N1CCC(/C=C\C(O)c2ccc(C(F)(F)F)cc2)C1]

### Synthetic Route:

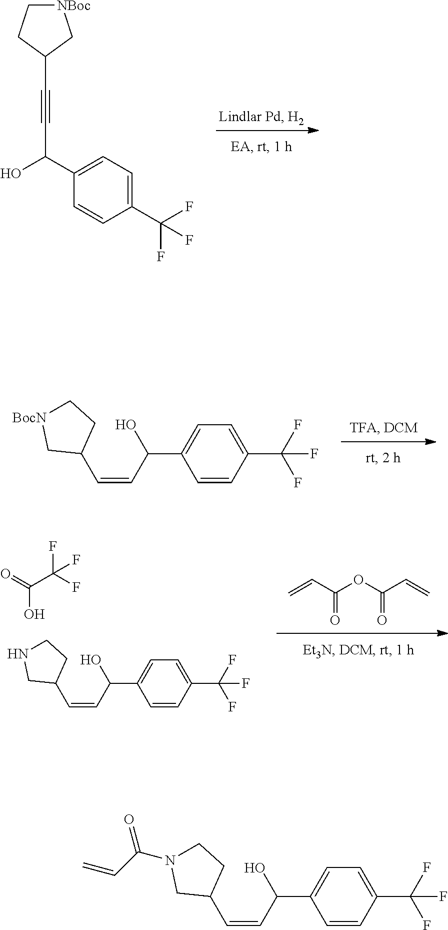

### Synthesis of tert-butyl (Z)-3-(3-hydroxy-3-(4-(trifluoromethyl)phenyl)prop-1-en-1-yl)pyrrolidine-1-carboxylate

tert-Butyl 3-(3-hydroxy-3-(4-(trifluoromethyl)phenyl)prop-1-yn-1-yl)pyrrolidine-1-carboxylate (500 mg, 1.35 mmol), a stir bar and EA (10 mL) were added to a 50 mL round-bottom flask and stirred until homogeneous, then treated with Lindlar catalyst (50 mg, 0.242 mmol) at room temperature. The resulting mixture was stirred for 1 hour under H₂ (1 atm) at room temperature, filtered and washed with EA (50 mL×5). The filtrate was concentrated under the reduced pressure. The residue obtained was purified by silica gel chromatography (0%-46% EA in PE) to give tert-butyl (Z)-3-(3-hydroxy-3-(4-(trifluoromethyl)phenyl)prop-1-en-1-yl)pyrrolidine-1-carboxylate (250 mg, 49.7% yield) as a green oil. MS (ESI, m/z) calcd. for C₁₉H₂₄F₃NO₃, 371.17, found, 370.10 [M−H]⁺.

### Synthesis of (Z)-3-(pyrrolidin-3-yl)-1-(4-(trifluoromethyl)phenyl)prop-2-en-1-ol 2,2,2-trifluoroacetate

tert-butyl (Z)-3-(3-hydroxy-3-(4-(trifluoromethyl)phenyl)prop-1-en-1-yl)pyrrolidine-1-carboxylate (100 mg, 0.269 mmol), a stir bar and DCM (2.5 mL) were added to a 25 mL round bottom flask and stirred until homogeneous, then treated with TFA (0.5 mL) dropwise at room temperature. The resulting mixture was stirred for 2 hours at room temperature and concentrated under the reduced pressure to give (Z)-3-(pyrrolidin-3-yl)-1-(4-(trifluoromethyl)phenyl)prop-2-en-1-ol 2,2,2-trifluoroacetate (170 mg, crude) as a brown oil. MS (ESI, m/z) calcd. for C₁₄H₁₆F₃NO, 271.12, found, 272.10 [M+H]⁺.

### Synthesis of (Z)-1-(3-(3-hydroxy-3-(4-(trifluoromethyl)phenyl)prop-1-en-1-yl)pyrrolidin-1-yl)prop-2-en-1-one

(Z)-3-(pyrrolidin-3-yl)-1-(4-(trifluoromethyl)phenyl)prop-2-en-1-ol 2,2,2-trifluoroacetate (130 mg, 0.337 mmol), a stir bar, Et₃N (121 mg, 1.20 mmol) and DCM (3.0 mL) were added to a 25 mL round-bottom flask and stirred until homogeneous, then treated with a solution of prop-2-enoyl prop-2-enoate (36 mg, 0.285 mmol) in DCM (0.5 mL) dropwise at 0° C. The resulting mixture was stirred for 1 hour at room temperature, then water (5 mL) was added and extracted with DCM (10 mL×3). The combined organic layers were washed with brine, dried over anhydrous sodium sulfate, filtered and concentrated. The residue was purified by Prep-HPLC (Column: XBridge Prep Pheny OBD Column, 19*250 mm, 5 μm; Mobile Phase A: Water (10 mmol/L NH₄HCO₃), Mobile Phase B: ACN; Flow rate: 25 mL/min mL/min; Gradient: 33% B to 63% B in 7 min; Wave Length: 254 nm nm; RT1(min): 6.66) to give (Z)-1-(3-(3-hydroxy-3-(4-(trifluoromethyl)phenyl)prop-1-en-1-yl)pyrrolidin-1-yl)prop-2-en-1-one as a white semi-solid (8 mg, 7.3% yield). MS (ESI, m/z) calcd. for C₁₇H₁₈F₃NO₂, 325.13, found, 326.15 [M+H]⁺. ¹H NMR (400 MHz, DMSO-d₆) δ 7.67 (s, 4H), 6.73-6.63 (m, 1H), 6.63-6.44 (m, 2H), 6.16-6.05 (m, 1H), 5.69-5.58 (m, 1H), 5.29-5.24 (m, 1H), 4.17-4.06 (m, 1H), 3.74-3.39 (m, 3H), 2.44-2.22 (m, 2H), 2.10-1.61 (m, 2H); ¹⁹F NMR (376 MHz, DMSO-d₆) δ −60.84

### Example 101: Synthesis of (Z)-1-(3-(3-methoxy-3-(4-(trifluoromethyl)phenyl)prop-1-en-1-yl)pyrrolidin-1-yl)prop-2-en-1-one

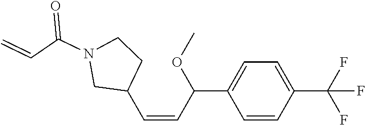 [SMILES: C=CC(=O)N1CCC(/C=C\C(OC)c2ccc(C(F)(F)F)cc2)C1]

### Synthetic Route:

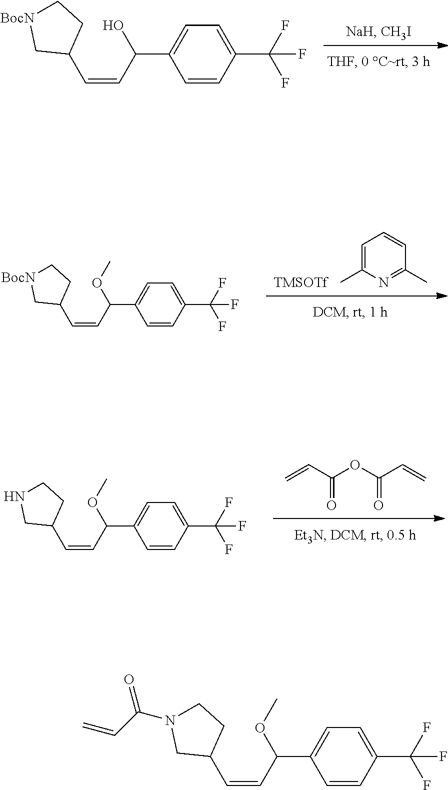

### Synthesis of tert-butyl (Z)-3-(3-methoxy-3-(4-(trifluoromethyl)phenyl)prop-1-en-1-yl)pyrrolidine-1-carboxylate

tert-Butyl (Z)-3-(3-hydroxy-3-(4-(trifluoromethyl)phenyl)prop-1-en-1-yl)pyrrolidine-1-carboxylate (100 mg, 0.269 mmol), a stir bar and THF (5 mL) were added to a 25 mL round-bottom flask and stirred until homogeneous, then treated with NaH (16 mg, 0.400 mmol) at 0° C. The resulting mixture was stirred for 30 min at 0° C., a solution of methyl iodide (76 mg, 0.535 mmol) in THF (0.5 mL) was added dropwise at 0° C., and stirring continued for 3 hours at room temperature, quenched with water (5 mL) and extracted with DCM (10 mL×3). The combined organic layers were washed with brine, dried over anhydrous sodium sulfate, filtered and concentrated. The residue obtained was purified by silica gel chromatography (0%-30% EA in PE) to give tert-butyl (Z)-3-(3-methoxy-3-(4-(trifluoromethyl)phenyl)prop-1-en-1-yl)pyrrolidine-1-carboxylate (70 mg, 67.5%) as a green oil. MS (ESI, m/z) calcd. for C₂₀H₂₆F₃NO₃, 385.19, found, 298.10 [M-^{t}Bu-OMe]⁺.

### Synthesis of (Z)-3-(3-methoxy-3-(4-(trifluoromethyl)phenyl)prop-1-en-1-yl)pyrrolidine

tert-Butyl (Z)-3-(3-methoxy-3-(4-(trifluoromethyl)phenyl)prop-1-en-1-yl)pyrrolidine-1-carboxylate (55 mg, 0.143 mmol), a stir bar, lutidine (61 mg, 0.569 mmol) and DCM (3 mL) were added to a 25 mL round-bottom flask and stirred until homogeneous, then treated with a solution of TMSOTf (63 mg, 0.283 mmol) in DCM (0.5 mL) dropwise at 0° C. The resulting mixture was stirred for 1 hour at room temperature, then water (5 mL) was added and extracted with DCM/MeOH (30 mL×3). The combined organic layers were washed with brine, dried over anhydrous sodium sulfate, filtered and concentrated under the reduced pressure to give (Z)-3-(3-methoxy-3-(4-(trifluoromethyl)phenyl)prop-1-en-1-yl)pyrrolidine (80 mg, crude) as a yellow oil. MS (ESI, m/z) calcd. for C₁₅H₁₈F₃NO, 285.13, found, 286.05 [M+H]⁺.

### Synthesis of (Z)-1-(3-(3-methoxy-3-(4-(trifluoromethyl)phenyl)prop-1-en-1-yl)pyrrolidin-1-yl)prop-2-en-1-one

(Z)-3-(3-Methoxy-3-(4-(trifluoromethyl)phenyl)prop-1-en-1-yl)pyrrolidine (70 mg, 0.245 mmol), a stir bar, Et₃N (49 mg, 0.484 mmol) and DCM (3 mL) were added to a 25 mL round-bottom flask and stirred until homogeneous, then treated with a solution of prop-2-enoyl prop-2-enoate (31 mg, 0.246 mmol) in DCM (0.5 mL) dropwise at 0° C. The resulting mixture was stirred for 0.5 hour at room temperature, diluted with water (10 mL) and extracted with DCM (20 mL×3). The combined organic layers were washed with brine, dried over anhydrous sulfate sodium, filtered and concentrated. The residue obtained was purified by PREP-HPLC (Column: XBridge Prep OBD C18 Column, 30*150 mm, 5 μm; Mobile Phase A: Water (10 mmol/L NH₄HCO₃), Mobile Phase B: ACN; Flow rate: 60 mL/min mL/min; Gradient: 38% B to 54% B in 10 min; Wave Length: 254 nm/220 nm nm; RT1(min): 8.42) to give (Z)-1-(3-(3-methoxy-3-(4-(trifluoromethyl)phenyl) prop-1-en-1-yl)pyrrolidin-1-yl)prop-2-en-1-one as a yellow oil (19.4 mg, 22.9% yield). MS (ESI, m/z) calcd. for C₁₈H₂₀F₃NO₂, 339.14, found, 340.20 [M+H]⁺. ¹H NMR (400 MHz, DMSO-d₆) δ 7.73 (d, J=8.1 Hz, 2H), 7.56 (d, J=8.1 Hz, 2H), 6.67-6.50 (m, 1H), 6.19-6.09 (m, 1H), 5.71-5.61 (m, 1H), 5.61-5.54 (m, 1H), 5.52-5.42 (m, 1H), 5.30-5.21 (m, 1H), 3.88-3.67 (m, 1H), 3.62-3.36 (m, 2H), 3.29-3.20 (m, 4H), 3.09-2.99 (m, 1H), 2.04-1.85 (m, 1H), 1.74-1.48 (m, 1H); ¹⁹F NMR (376 MHz, DMSO-d₆) δ −60.86.

### Example 102: Synthesis of (E)-1-(3-(3-methoxy-3-(4-(trifluoromethyl)phenyl)prop-1-en-1-yl)pyrrolidin-1-yl)prop-2-en-1-one

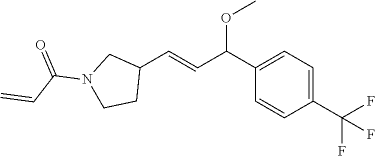 [SMILES: C=CC(=O)N1CCC(/C=C/C(OC)c2ccc(C(F)(F)F)cc2)C1]

### Synthetic Route:

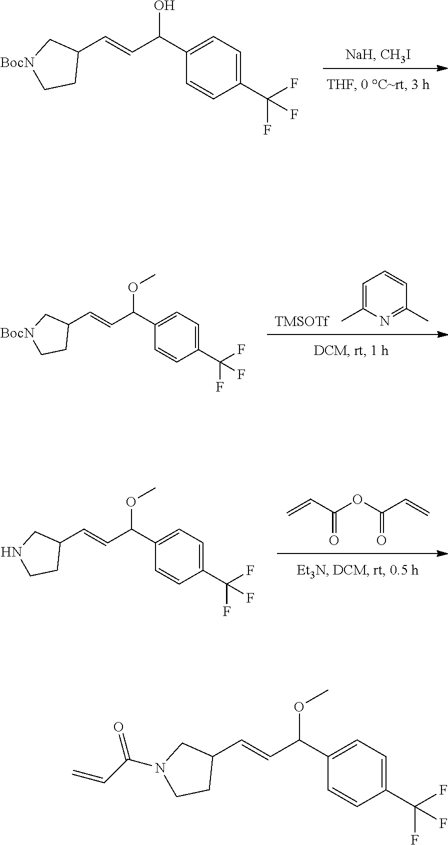

### Synthesis of tert-butyl (E)-3-(3-methoxy-3-(4-(trifluoromethyl)phenyl)prop-1-en-1-yl)pyrrolidine-1-carboxylate

tert-butyl (E)-3-(3-hydroxy-3-(4-(trifluoromethyl)phenyl)prop-1-en-1-yl)pyrrolidine-1-carboxylate (100 mg, 0.269 mmol), a stir bar and THF (5 mL) were added to a 25 mL round-bottom flask and stirred until homogeneous, then treated with NaH (16 mg, 0.400 mmol) at 0° C. The resulting mixture was stirred for 30 min at 0° C., and a solution of methyl iodide (76 mg, 0.535 mmol) in THF (0.5 mL) was added dropwise at 0° C. The reaction continued to stir for 3 hours at room temperature, then quenched with water (5 mL) and extracted with DCM (10 mL×3). The combined organic layers were washed with brine, dried over anhydrous sodium sulfate, filtered and concentrated. The residue was purified by silica gel chromatography (0%-30% EA in PE) to give tert-butyl (E)-3-(3-methoxy-3-(4-(trifluoromethyl)phenyl)prop-1-en-1-yl)pyrrolidine-1-carboxylate (50 mg, 48.2%) as a green oil. MS (ESI, m/z) calcd. for C₂₀H₂₆F₃NO₃, 385.19, found, 298.10 [M-^{t}Bu-OMe]⁺.

### Synthesis of (E)-3-(3-methoxy-3-(4-(trifluoromethyl)phenyl)prop-1-en-1-yl)pyrrolidine

tert-butyl (E)-3-(3-methoxy-3-(4-(trifluoromethyl)phenyl)prop-1-en-1-yl)pyrrolidine-1-carboxylate (50 mg, 0.130 mmol), a stir bar, lutidine (56 mg, 0.523 mmol) and DCM (3 mL) were added to a 25 mL round bottom flask and stirred until homogeneous, then treated with a solution of TMSOTf (58 mg, 0.261 mmol) in DCM (0.5 mL) dropwise at 0° C. The resulting mixture was stirred for 1 hour at room temperature, then water (5 mL) was added and extracted with DCM/MeOH (30 mL×3). The combined organic layers were washed with brine, dried over anhydrous sodium sulfate, filtered and concentrated to give (E)-3-(3-methoxy-3-(4-(trifluoromethyl)phenyl)prop-1-en-1-yl)pyrrolidine (46 mg, crude) as a green oil. MS (ESI, m/z) calcd. for C₁₅H₁₈F₃NO, 285.13, found, 286.10 [M+H]⁺.

### Synthesis of (E)-1-(3-(3-methoxy-3-(4-(trifluoromethyl)phenyl)prop-1-en-1-yl)pyrrolidin-1-yl)prop-2-en-1-one

(E)-3-(3-Methoxy-3-(4-(trifluoromethyl)phenyl)prop-1-en-1-yl)pyrrolidine (46 mg, 0.161 mmol), a stir bar, Et₃N (32 mg, 0.316 mmol) and DCM (3 mL) were added to a 25 mL round bottom flask and stirred until homogeneous, then treated with a solution of prop-2-enoyl prop-2-enoate (20 mg, 0.159 mmol) in DCM (0.5 mL) dropwise at 0° C. The resulting mixture was stirred for 0.5 h at r.t. diluted with water (10 mL) and extracted with DCM (20 mL×3). The combined organic layers were washed with brine, dried over anhydrous sulfate sodium, filtered and concentrated under vacuum pressure. The residue was purified by PREP-HPLC (Column: XBridge Prep OBD Cis Column, 30*150 mm, 5 μm; Mobile Phase A: Water (10 mmol/L NH₄HCO₃), Mobile Phase B: ACN; Flow rate: 60 mL/min mL/min; Gradient: 37% B to 53% B in 10 min; Wave Length: 254 nm/220 nm nm; RT1(min): 8.95) to give (E)-1-(3-(3-methoxy-3-(4-(trifluoromethyl)phenyl) prop-1-en-1-yl)pyrrolidin-1-yl)prop-2-en-1-one (8.8 mg, 16% yield). MS (ESI, m/z) calcd. for C₁₈H₂₀F₃NO₂, 339.14, found, 340.20 [M+H]⁺. ¹H NMR (400 MHz, DMSO-d₆) δ 7.73 (d, J=7.9 Hz, 2H), 7.53 (d, J=8.0 Hz, 2H), 6.64-6.50 (m, 1H), 6.17-6.06 (m, 1H), 5.88-5.76 (m, 1H), 5.70-5.53 (m, 2H), 4.79 (d, J=7.2 Hz, 1H), 3.80-3.43 (m, 3H), 3.23 (s, 4H), 3.15-2.78 (m, 1H), 2.10-1.92 (m, 1H), 1.85-1.56 (m, 1H); ¹⁹F NMR (376 MHz, DMSO-d₆) δ −60.86.

### Example 103: Synthesis of 1-((S)-3-((S,E)-1-hydroxy-3-(4-(trifluoromethyl)phenyl)allyl) pyrrolidin-1-yl)prop-2-en-1-one

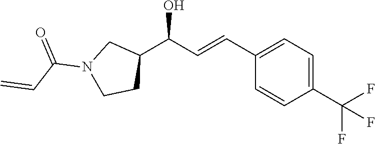 [SMILES: C=CC(=O)N1CC[C@H]([C@@H](O)/C=C/c2ccc(C(F)(F)F)cc2)C1]

### Synthetic Route:

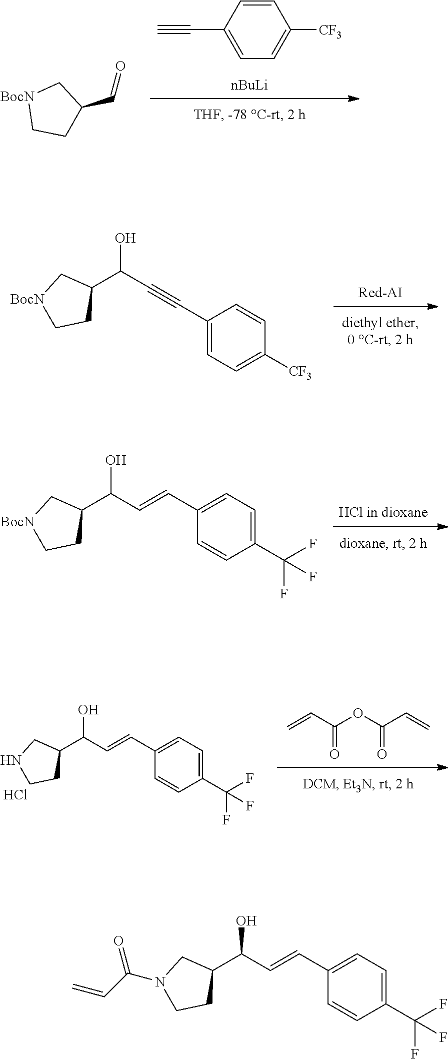

### Synthesis of tert-butyl (3S)-3-(1-hydroxy-3-(4-(trifluoromethyl)phenyl)prop-2-yn-1-yl) pyrrolidine-1-carboxylate

1-Ethynyl-4-(trifluoromethyl)benzene (1.88 g, 11.0 mmol), a stir bar, THF (20 mL) were added to a 100 mL three-necked flask and stirred until homogeneous, then treated with n-butyllithium (4.82 mL, 2.5 M, 12.0 mmol) dropwise at −78° C. under nitrogen atmosphere. The resulting mixture was stirred at −78° C. for 30 min under nitrogen atmosphere, then a solution of tert-butyl (3S)-3-formylpyrrolidine-1-carboxylate (2 g, 10.0 mmol) in THF (10 mL) was added and stirring continued for 2 h at room temperature. The mixture was quenched with NH4Cl aqueous solution and extracted with EA (100 mL×2). The organic layers were combined, dried over anhydrous Na2SO4, filtered and concentrated. The residue obtained was purified by silica gel chromatography (EA/PE=0-35%) to afford tert-butyl (3S)-3-(1-hydroxy-3-(4-(trifluoromethyl) phenyl)prop-2-yn-1-yl)pyrrolidine-1-carboxylate as yellow oil (3.44 g, 92.8% yield). MS (ESI, m/z) calcd. for C₁₉H₂₂F₃NO₃, 369.16, found, 314.00 [M-^{t}Bu+H]⁺.

### Synthesis of tert-butyl (3S)-3-((E)-1-hydroxy-3-(4-(trifluoromethyl)phenyl)allyl)pyrrolidine-1-carboxylate

tert-Butyl (3S)-3-(1-hydroxy-3-(4-(trifluoromethyl)phenyl)prop-2-yn-1-yl)pyrrolidine-1-carboxylate (3.34 g, 9.04 mmol), a stir bar, diethyl ether (40 mL) were added to a 100 mL three necked flask and stirred until homogeneous, then treated with the mixture of Red-Al (3.66 g, 12.7 mmol) in diethyl ether (26 mL) dropwise at 0° C. under nitrogen atmosphere. The resulting mixture was warmed to room temperature and stirred for 2 h, then quenched with NH₄Cl aqueous solution. The resulting mixture was extracted with EA (100 mL×2). The combined organic layers were washed with brine, dried over anhydrous Na₂SO₄, filtered and concentrated. The residue obtained was purified by silica gel chromatography (0-50% EA/PE) to afford tert-butyl (3S)-3-((E)-1-hydroxy-3-(4-(trifluoromethyl)phenyl)allyl)pyrrolidine-1-carboxylate as a white solid (2.61 g, 77.7% yield). MS (ESI, m/z) calcd. for C₁₉H₂₄F₃NO₃, 371.17, found, 316.05 [M-^{t}Bu+H]⁺.

### Synthesis of ((E)-1-((S)-pyrrolidin-3-yl)-3-(4-(trifluoromethyl)phenyl)prop-2-en-1-ol hydrochloride

tert-Butyl (3S)-3-((E)-1-hydroxy-3-(4-(trifluoromethyl)phenyl)allyl)pyrrolidine-1-carboxylate (400 mg, 1.08 mmol), dioxane (8 mL) and a stir bar were added to a 50 mL round-bottom flask and stirred until homogenous, then treated with HCl in 1,4-dioxane (4 mL, 4M) dropwise at room temperature. The resulting mixture was stirred for 1 hour at room temperature, then concentrated under the reduced pressure to afford (E)-1-((S)-pyrrolidin-3-yl)-3-(4-(trifluoromethyl) phenyl)prop-2-en-1-ol hydrochloride as a yellow solid (450 mg, crude). MS (ESI, m/z) calcd. for C₁₄H₁₇F₃NO, 271.28, found, 272.05 [M+H]⁺.

### Synthesis of 1-((S)-3-((S,E)-1-hydroxy-3-(4-(trifluoromethyl)phenyl)allyl)pyrrolidin-1-yl)prop-2-en-1-one

(E)-1-((S)-Pyrrolidin-3-yl)-3-(4-(trifluoromethyl) phenyl)prop-2-en-1-ol hydrochloride (400 mg, 1.300 mmol), DCM (8 mL), TEA (658 mg, 6.502 mmol) and a stir bar were added to a 20 mL vial and stirred until homogenous, then treated with a solution of prop-2-enoyl prop-2-enoate (196 mg, 1.554 mmol) in DCM (1 mL) at 0° C. The resulting mixture was stirred for 1 h at r.t, then diluted with water (50 mL) and extracted with DCM (50 mL×2). The combined organic layers were washed with brine, dried over anhydrous sodium sulfate, filtered and concentrated to dryness. The resulting residue was purified by Prep-HPLC with (Column: XSelect CSH C18 Column, 19*250 mm, 5 μm; Mobile Phase A: Water (0.1% FA), Mobile Phase B: ACN; Flow rate: 25 mL/min mL/min; Gradient: 33% B to 57% B in 10 min; Wave Length: 254 nm nm; RT1(min): 8.36, RT2(min): 8.88) to give two products. The first eluting fractions were lyophilized to afford 1-((S)-3-((S E)-1-hydroxy-3-(4-(trifluoromethyl)phenyl)allyl)pyrrolidin-1-yl)prop-2-en-1-one as a white solid (24.2 mg, 5.7% yield). MS (ESI, m/z) calcd. for C₁₇H₁₈F₃NO₂, 325.10, found, 326.05 [M+H]⁺. ¹H NMR (400 MHz, DMSO-d₆) δ 7.68 (s, 4H), 6.73-6.62 (m, 1H), 6.62-6.46 (m, 2H), 6.16-6.05 (m, 1H), 5.68-5.59 (m, 1H), 5.28 (dd, J=8.2, 4.8 Hz, 1H), 4.16-4.08 (m, 1H), 3.72-3.60 (m, 1H), 3.59-3.43 (m, 2H), 3.30-3.12 (m, 1H), 2.45-2.24 (m, 1H), 2.08-1.92 (m, 1H), 1.89-1.67 (m, 1H); ¹⁹F NMR (376 MHz, DMSO-d₆) δ −60.82.

### Example 104: Synthesis of 1-((S)-3-((R,E)-1-hydroxy-3-(4-(trifluoromethyl)phenyl)allyl) pyrrolidin-1-yl)prop-2-en-1-one

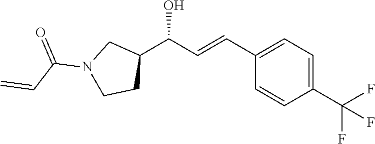 [SMILES: C=CC(=O)N1CC[C@H]([C@H](O)/C=C/c2ccc(C(F)(F)F)cc2)C1]

### Synthetic Route:

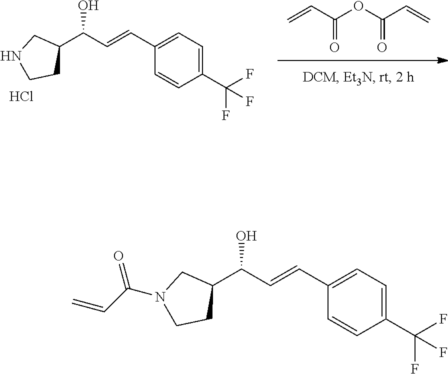

### Synthesis of 1-((S)-3-((R,E)-1-hydroxy-3-(4-(trifluoromethyl)phenyl)allyl)pyrrolidin-1-yl)prop-2-en-1-one

(E)-1-((S)-pyrrolidin-3-yl)-3-(4-(trifluoromethyl) phenyl)prop-2-en-1-ol hydrochloride (400 mg, 1.300 mmol), DCM (8 mL), TEA (658 mg, 6.502 mmol) and a stir bar were added to a 20 mL vial and stirred until homogenous, then treated with a solution of prop-2-enoyl prop-2-enoate (196 mg, 1.554 mmol) in DCM (1 mL) at 0° C. The resulting mixture was stirred for 1 h at r.t, then diluted with water (50 mL) and extracted with DCM (50 mL×2). The combined organic layers were washed with brine, dried over anhydrous sodium sulfate, filtered and concentrated to dryness. The resulting residue was purified by Prep-HPLC with (Column: XSelect CSH C18 Column, 19*250 mm, 5 μm; Mobile Phase A: Water (0.1% FA), Mobile Phase B: ACN; Flow rate: 25 mL/min mL/min; Gradient: 33% B to 57% B in 10 min; Wave Length: 254 nm nm; RT1(min): 8.36. RT2(min): 8.88) to give two products. The second eluting fractions were lyophilized to afford 1-((S)-3-((R,E)-1-hydroxy-3-(4-(trifluoromethyl)phenyl)allyl)pyrrolidin-1-yl)prop-2-en-1-one as a white solid (33.5 mg, 7.9% yield). MS (ESI, m/z) calcd. for C₁₇H₁₈F₃NO₂, 325.10, found, 326.05 [M+H]⁺. ¹H NMR (400 MHz, DMSO-d₆) δ 7.68 (d, J=1.4 Hz, 4H), 6.72-6.65 (m, 1H), 6.63-6.47 (m, 2H), 6.16-6.07 (m, 1H), 5.69-5.62 (m, 1H), 5.30 (dd, 0.1=12.0, 4.8 Hz, 1H), 4.15-4.07 (m, 1H), 3.74-3.63 (m, 1H), 3.59-3.39 (m, 2H), 3.32-3.21 (m, 1H), 2.43-2.23 (m, 1H), 1.98-1.62 (m, 2H); ¹⁹F NMR (376 MHz, DMSO-d₆) δ −60.83.

### Example 105: Synthesis of 1-(3-(4-(trifluoromethyl)phenyl)-2-oxa-7-azaspiro[4.4]non-3-en-7-yl)prop-2-en-1-one

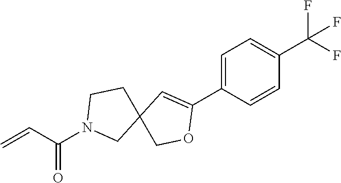 [SMILES: C=CC(=O)N1CCC2(C=C(c3ccc(C(F)(F)F)cc3)OC2)C1]

### Synthetic Route:

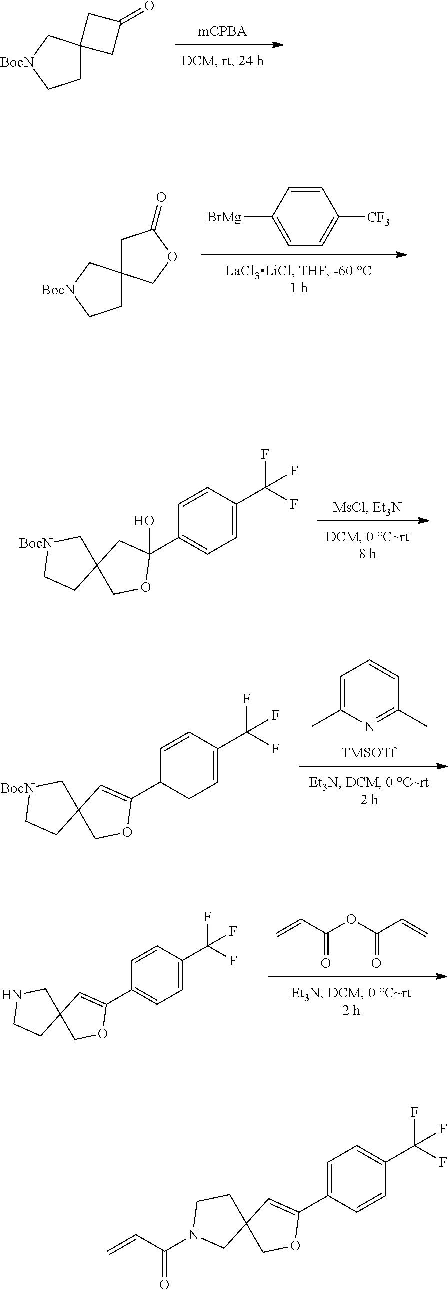

### Synthesis of tert-butyl 3-oxo-2-oxa-7-azaspiro[4.4]nonane-7-carboxylate

tert-Butyl 2-oxo-6-azaspiro[3.4]octane-6-carboxylate (2.0 g, 8.88 mmol), a stir bar and DCM (10 mL) were added to a 40 mL vial and stirred until homogenous, then treated with mCPBA (3.0 g, 17.4 mmol) portions by portions at room temperature. The resulting mixture was stirred for 24 hours at room temperature, then saturated Na₂CO₃ (aq.) solution was added and extracted with DCM (3×30 mL). The combined organic layers were washed with saturated Na₂CO₃ (aq.), dried over anhydrous Na₂SO₄ and concentrated. The obtained residue was purified by reverse phase chromatography (0%~80% ACN/10 mM NH₄HCO₃ water) to afford tert-butyl 3-oxo-2-oxa-7-azaspiro[4.4]nonane-7-carboxylate (1.5 g, 70% yield) as a white solid. MS (ESI, m/z) calcd. for C₁₂H₁₉NO₄ 241.13, found, 186.10 [M-^{t}Bu+H]⁺.

### Synthesis of tert-butyl 3-hydroxy-3-[4-(trifluoromethyl)phenyl]-2-oxa-7-azaspiro[4.4]nonane-7-carboxylate

tert-Butyl 3-oxo-2-oxa-7-azaspiro[4.4]nonane-7-carboxylate (1.4 g, 5.80 mmol), a stir bar and THF (15 mL) were added to a 100 mL three-necked bottomed flask and stirred until homogeneous, then treated with LaCl₃·2LiCl (10.4 mL, 6.24 mmol, 0.6 M in THF) dropwise at −60° C. under nitrogen atmosphere. The resulting mixture was stirred for 30 min at −60° C. under nitrogen atmosphere, then bromo[4-(trifluoromethyl)phenyl]magnesium (10.4 mL, 6.24 mmol, 0.6 M in Et₂O) was added dropwise under nitrogen atmosphere at −60° C. The resulting mixture continued to stir for 1 hour at −60° C., then quenched with saturated NH₄Cl (aq.) solution and extracted with DCM (30 mL×3). The combined organic layers were washed with brine, dried over anhydrous Na₂SO₄ and concentrated. The obtained residue was purified by silica gel column (0%~50% EA in PE) to afford tert-butyl 3-hydroxy-3-[4-(trifluoromethyl)phenyl]-2-oxa-7-azaspiro[4.4]nonane-7-carboxylate (880 mg, 39.2% yield) as a light orange solid. MS (ESI, m/z) calcd. for C₁₉H₂₄F₃NO₄, 387.16, found, 288.20[M-Boc+H]⁺.

### Synthesis of tert-butyl 3-(4-(trifluoromethyl)cyclohexa-2,4-dien-1-yl)-2-oxa-7-azaspiro[4.4]non-3-ene-7-carboxylate

tert-butyl 3-hydroxy-3-[4-(trifluoromethyl)phenyl]-2-oxa-7-azaspiro[4.4]nonane-7-carboxylate (330 mg, 0.85 mmol), a stir bar, DCM (4.0 mL) and Et₃N (430 mg, 4.25 mmol) were added to a 8 mL vial and stirred until homogeneous, then treated with MsCl (327 mg, 2.86 mmol) in DCM (1.0 mL) dropwise at 0° C. The resulting mixture was stirred overnight at room temperature. The reaction was quenched with water and extracted with DCM (15 mL×3). The combined organic layers were washed with brine, dried over anhydrous Na₂SO₄ and concentrated to afford tert-butyl 3-(4-(trifluoromethyl)cyclohexa-2,4-dien-1-yl)-2-oxa-7-azaspiro[4.4]non-3-ene-7-carboxylate (550 mg, crude) as a yellow oil. MS (ESI, m/z) calcd. for C₁₉H₂₂F₃NO₃, 369.15, found, 314.15[M-^{t}Bu+H]⁺.

### Synthesis of 3-(4-(trifluoromethyl)phenyl)-2-oxa-7-azaspiro[4.4]non-3-ene

tert-butyl 3-(4-(trifluoromethyl)cyclohexa-2,4-dien-1-yl)-2-oxa-7-azaspiro[4.4]non-3-ene-7-carboxylate (250 mg, 0.68 mmol), a stir bar and DCM (2.0 mL) were added to a 8 mL vial and stirred until homogeneous, then treated with a solution of lutidine (226 mg, 2.11 mmol) in DCM (0.5 mL) and a solution of TMSOTf (219 mg mL, 0.99 mmol) in DCM (0.5 mL) dropwise at 0° C. under nitrogen atmosphere. The resulting mixture was stirred for 2 hours at 0° C. under nitrogen atmosphere, then water was added and extracted with DCM (10 mL×3). The combined organic layers were washed with brine, dried over anhydrous Na₂SO₄ and concentrated to afford 3-(4-(trifluoromethyl)phenyl)-2-oxa-7-azaspiro[4.4]non-3-ene (230 mg, crude) as a light yellow oil. MS (ESI, m/z) calcd. for C₁₄H₁₄F₃NO, 269.10, found, 270.15 [M+H]⁺.

### Synthesis of 1-(3-[4-(trifluoromethyl)phenyl]-2-oxa-7-azaspiro[4.4]non-3-en-7-ylprop-2-en-1-one

3-(4-(Trifluoromethyl)phenyl)-2-oxa-7-azaspiro[4.4]non-3-ene (220 mg, 0.82 mmol), a stir bar, DCM (3.5 mL) and Et₃N (249 mg, 2.46 mmol) were added to a 40 mL vial and stirred until homogeneous, then treated with a solution of prop-2-enoyl prop-2-enoat (124 mg, 0.98 mmol) in DCM (0.5 mL) dropwise at 0° C. The resulting mixture was stirred for 1 hour at 0° C., then water was added and extracted with DCM (10 mL×3). The combined organic layers were washed with brine, dried over anhydrous Na₂SO₄ and concentrated. The obtained residue was purified by Prep-HPLC (Column: XBridge BEH Shield RP18 Column, 19*250 mm, 5 μm; Mobile Phase A: Water (10 mmol/L NH₄HCO₃). Mobile Phase B: ACN; Flow rate: 25 mL/min mL/min; Gradient: 42% B to 72% B in 7 min; Wave Length: 254 nm/220 nm nm; RT1(min): 6.57) to afford 1-(3-[4-(trifluoromethyl)phenyl]-2-oxa-7-azaspiro[4.4]non-3-en-7-ylprop-2-en-1-one (26.0 mg, 9.8% yield) as a white solid, MS (ESI, m/z) calcd. for C₁₇H₁₆F₃NO₂ 323.11, found 324.10[M+H]⁺. ¹H NMR (500 MHz, DMSO-d₆) δ 7.82-7.72 (m, 4H), 6.59 (td, J=17.0, 10.3 Hz, 1H), 6.15 (ddd, J=16.7, 7.0, 2.5 Hz, 1H), 5.93 (d, J=6.4 Hz, 1H), 5.68 (ddd, J=17.6, 10.3, 2.4 Hz, 1H), 4.42 (dd, J=9.3, 2.0 Hz, 1H), 4.38 (dd, J=9.2, 7.1 Hz, 1H), 3.78-3.67 (m, 1H), 3.65-3.37 (m, 3H), 2.14-1.91 (m, 2H).

### Example 106: Synthesis of (E)-1-(2-(4-(trifluoromethyl)styryl)-6-azaspiro[3.4]octan-6-yl)prop-2-en-1-one

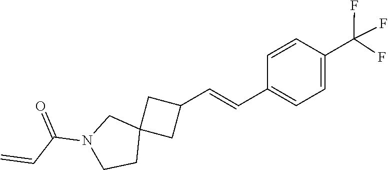 [SMILES: C=CC(=O)N1CCC2(CC(/C=C/c3ccc(C(F)(F)F)cc3)C2)C1]

### Synthetic Route:

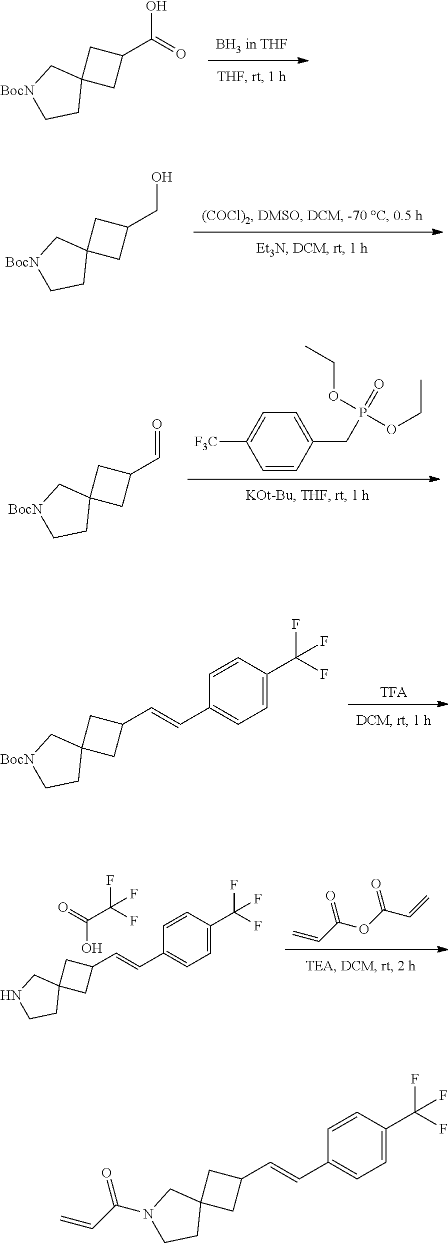

### Synthesis of tert-butyl 2-(hydroxymethyl)-6-azaspiro[3.4]octane-6-carboxylate

6-(tert-Butoxycarbonyl)-6-azaspiro[3.4]octane-2-carboxylic acid (2 g, 7.83 mmol), a stir bar and THF (33 mL) were added to a 250 mL round bottom flask and stirred until homogeneous, then treated with BH₃ in THF (39.3 mL, 1M, 39.3 mmol) at 0° C. The resulting mixture was stirred at room temperature for 1 hour and quenched with MeOH (5 mL), extracted with EA (3×200 mL). The combined organic layers were washed with brine, dried over anhydrous sodium sulfate, filtered and concentrated under the reduced pressure. The residue obtained was purified by silica gel chromatography (0-70% EA/PE) to afford tert-butyl 2-(hydroxymethyl)-6-azaspiro[3.4]octane-6-carboxylate as a colorless oil (1.7 g, 89.9% yield). MS (ESI, m/z) calcd. for C₁₃H₂₃NO₃, 241.16, found 186.05 [M-^{t}Bu+H]⁺.

### Synthesis of tert-butyl 2-formyl-6-azaspiro[3.4]octane-6-carboxylate

Oxalyl chloride (526 mg, 4.14 mmol), a stir bar, DMSO (647 mg, 8.29 mmol) and DCM (8 mL) were added to a 100 mL round-bottom flask and stirred until homogeneous. The resulting mixture was stirred at −70° C. under nitrogen atmosphere for 0.5 hour, then treated with tert-butyl 2-(hydroxymethyl)-6-azaspiro[3.4]octane-6-carboxylate (500 mg, 2.07 mmol) in DCM (1 mL) dropwise. The resulting mixture was stirred at −70° C. for 1 hour, then treated with triethylamine (1.68 g, 16.6 mmol) in DCM (1 mL) dropwise. The resulting mixture was stirred at room temperature for 1 hour, then quenched with H₂O and extracted with DCM (100 mL) twice. The combined organic layers were washed with brine, dried over anhydrous sodium sulfate, filtered and concentrated under the reduced pressure. The residue obtained was purified by silica gel chromatography (0-30% EA/PE) to afford tert-butyl 2-formyl-6-azaspiro[3.4]octane-6-carboxylate as a yellow oil (0.485 g, 97.8% yield). MS (ESI, m/z) calcd. for C₁₃H₂₁NO₃, 239.15, found, 184.05 [M-^{t}Bu+H]⁺.

### Synthesis of tert-butyl (E)-2-(4-(trifluoromethyl)styryl)-6-azaspiro[3.4]octane-6-carboxylate

tert-Butyl 2-formyl-6-azaspiro[3.4]octane-6-carboxylate (482 mg, 2.01 mmol), a stir bar, diethyl [4-(trifluoromethyl)phenyl]methylphosphonate (542 mg, 1.83 mmol) and THF (15 mL) were added to a 100 mL round-bottom flask and stirred until homogeneous, then treated with potassium tert-butoxide (246 mg, 2.20 mmol) at 0° C. The resulting mixture was stirred at room temperature for 1 hour, then quenched with H₂O (20 mL) and extracted with EA (3×50 mL). The combined organic layers were washed with brine, dried over anhydrous sodium sulfate, filtered and concentrated. The residue obtained was purified by silica gel chromatography (0-70% EA/PE) to afford tert-butyl (E)-2-(4-(trifluoromethyl)styryl)-6-azaspiro[3.4]octane-6-carboxylate as a yellow oil (0.488 g, 69.9% yield). MS (ESI, m/z) calcd. for C₂₁H₂₆F₃NO₂, 381.19, found, 326.05 [M-^{t}Bu+H]⁺.

Synthesis of (E)-2-(4-(trifluoromethyl)styryl)-6-azaspiro[3.4]octane 2,2,2-trifluoroacetate tert-Butyl (E)-2-(4-(trifluoromethyl)styryl)-6-azaspiro[3.4]octane-6-carboxylate (480 mg, 1.26 mmol), a stir bar and DCM (5 mL) were added to a 40 mL vial and stirred until homogenous, then treated with TFA (1 mL). The resulting mixture was stirred at room temperature for 1 hour and concentrated under the reduced pressure to afford (E)-2-(4-(trifluoromethyl)styryl)-6-azaspiro[3.4]octane 2,2,2-trifluoroacetate as a white oil (0.480 g, crude). MS (ESI, m/z) calcd. for C₁₆H₁₈F₃N, 281.05, found, 282.05 [M+H]⁺.

### Synthesis of (E)-1-(2-(4-(trifluoromethyl)styryl)-6-azaspiro[3.4]octan-6-yl)prop-2-en-1-one

2-[(E)-2-[4-(Trifluoromethyl)phenyl]ethenyl]-6-azaspiro[3.4]octane 2,2,2-trifluoroacetate (200 mg, 0.506 mmol), triethylamine (256 mg, 2.53 mmol), a stir bar and DCM (15 mL) were added to a 40 mL vial and stirred until homogenous, then treated with a solution of prop-2-enoyl prop-2-enoate (77 mg, 0.607 mmol) in DCM (1 mL) dropwise. The resulting mixture was stirred at room temperature for 2 hours. The reaction was quenched with H₂O (20 mL) and extracted with DCM (3×50 mL). The organic layers were combined, dried over Na₂SO₄, filtered and concentrated. The residue obtained was purified by Prep-HPLC (Column: XSelect CSH C18 Column, 30*150 mm, 5 μm; Mobile Phase A: Water (0.1% FA), Mobile Phase B: ACN; Flow rate: 60 mL/min mL/min; Gradient: 46% B to 76% B in 7 min; Wave Length: 254 nm nm; RT1(min): 6.77) to afford (E)-1-(2-(4-(trifluoromethyl)styryl)-6-azaspiro[3.4]octan-6-yl)prop-2-en-1-one (32.6 mg, 19.2%) as a colorless oil. MS (ESI, m/z) calcd. for C₁₉H₂₀F₃NO, 335.14, found, 336.05 [M+H]⁺. ¹H NMR (400 MHz, DMSO-d₆) δ 7.78-7.38 (m, 4H), 6.71-6.37 (m, 3H), 6.19-6.06 (m, 1H), 5.70-5.62 (m, 1H), 3.69-3.35 (m, 4H), 3.21-3.07 (m, 1H), 2.34-2.11 (m, 2H), 2.09-1.76 (m, 4H); ¹⁹F NMR (376 MHz, DMSO-d₆) δ −60.23.

### BIOLOGICAL EXAMPLES

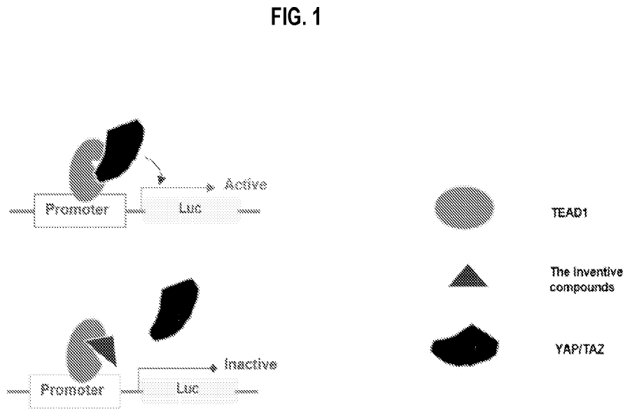

FIG. 1 is a schematic illustration of the mechanism of TEAD1, 2, 3, and/or 4 inhibition by the disclosed compounds, which is the theoretical basis for the development of a TEAD-YAP/TAZ cell-based functional assay to characterize the pharmacological properties of the compounds provided herein.

### Biological Example 1

### MCF-7 Reporter Assay

The ability of compounds to inhibit TEAD-dependent transcription was assessed in the MCF7-TEAD-Luc reporter assay (BPS #60618). Cells were seeded at 10,000 cells in 200 μL per well into white wall, clear bottom 96-well plates (Corning #3610) and incubated at 37° C. for 24 hours, then treated with serial diluted compounds overnight, and luciferase signal was measured using the ONE-Step Luciferase Assay System (BPS #60690) using an EnSight Multimode plate reader (PerkinElmer, model #MLDHH34000000, integration time 100 ms) with Kaleido 3.0. software. For data analysis, dose-dependent curves were plotted as “% inhibition” (% of DMSO-treated control wells), and were fitted by four-parametric model in GraphPad Prism to obtain IC₅₀ values.

TABLE 2
The pharmacological data obtained from
the TEAD1-Luciferase reporter assay.

| | | TEAD1-Luc | TEAD1-Luc |
|---|---|---|---|
| | | MCF7: Avg | MCF7: Max |
| Example | Compound | IC₅₀ | Inhibition |
| 1 | 1 | ++++ | A |
| 2 | 2 | ++++ | A |
| 3 | 3 | ++++ | B |
| 4 | 4 | ++++ | A |
| 5 | 5 | ++++ | B |
| 6 | 6 | +++ | B |
| 7 | 7 | ++++ | A |
| 8 | 8 | ++++ | A |
| 9 | 9 | +++ | A |
| 10 | 10 | ++++ | A |
| 11 | 11 | ++++ | A |
| 12 | 12 | +++ | A |
| 13 | 13 | ++++ | A |
| 14 | 14 | +++ | B |
| 15 | 15 | ++++ | A |
| 16 | 16 | ++++ | A |
| 17 | 17 | ++++ | B |
| 18 | 18 | ++++ | A |
| 19 | 19 | +++ | C |
| 20 | 20 | ++ | B |
| 21 | 21 | ++++ | A |
| 22 | 22 | +++ | B |
| 23 | 23 | +++ | B |
| 24 | 24 | + | C |
| 25 | 25 | ++ | B |
| 26 | 26 | +++ | B |
| 27 | 27 | ++ | A |
| 28 | 28 | +++ | B |
| 29 | 29 | +++ | A |
| 30 | 30 | +++ | A |
| 31 | 31 | ++ | A |
| 32 | 32 | ++ | A |
| 33 | 33 | ++ | A |
| 34 | 34 | +++ | A |
| 35 | 35 | +++ | A |
| 36 | 36 | +++ | A |
| 37 | 37 | +++ | A |
| 38 | 38 | ++++ | A |
| 39 | 39 | +++ | A |
| 40 | 40 | + | A |
| 41 | 41 | +++ | A |
| 42 | 42 | ++ | A |
| 43 | 43 | ++ | B |
| 44 | 44 | +++ | A |
| 45 | 45 | +++ | A |
| 46 | 46 | +++ | B |
| 47 | 47 | ++++ | C |
| 48 | 48 | ++ | B |
| 49 | 49 | ++ | B |
| 50 | 50 | ++++ | B |
| 51 | 51 | +++ | B |
| 52 | 52 | +++ | A |
| 53 | 53 | ++ | B |
| 54 | 54 | ++ | B |
| 55 | 55 | + | C |
| 56 | 56 | +++ | A |
| 57 | 57 | +++ | A |
| 58 | 58 | ++++ | B |
| 59 | 59 | +++ | A |
| 60 | 60 | +++ | A |
| 61 | 61 | +++ | A |
| 62 | 62 | +++ | B |
| 63 | 63 | +++ | A |
| 64 | 64 | +++ | A |
| 65 | 65 | ++ | A |
| 66 | 66 | +++ | A |
| 67 | 67 | ++++ | A |
| 68 | 68 | ++ | B |
| 69 | 69 | ++ | B |
| 70 | 70 | +++ | A |
| 71 | 71 | +++ | B |
| 72 | 72 | +++ | B |
| 73 | 73 | +++ | A |
| 74 | 74 | ++++ | B |
| 75 | 75 | +++ | A |
| 76 | 76 | +++ | A |
| 77 | 77 | +++ | B |
| 78 | 78 | ++ | A |
| 79 | 79 | +++ | B |
| 80 | 80 | +++ | A |
| 81 | 81 | ++ | B |
| 82 | 82 | +++ | A |
| 83 | 83 | +++ | A |
| 84 | 84 | +++ | A |
| 85 | 85 | ++++ | A |
| 86 | 86 | +++ | B |
| 87 | 87 | ++ | B |
| 88 | 88 | ++++ | B |
| 89 | 89 | +++ | A |
| 90 | 90 | +++ | B |
| 91 | 91 | + | B |
| 92 | 92 | + | B |
| 93 | 93 | +++ | B |
| 94 | 94 | +++ | B |
| 95 | 95 | ++ | B |
| 96 | 96 | +++ | B |
| 97 | 97 | ++ | C |
| 98 | 98 | + | C |
| 99 | 100 | +++ | B |
| 100 | 99 | +++ | B |
| 101 | 101 | ++ | B |
| 102 | 102 | +++ | C |
| 103 | 103 | ++ | B |
| 104 | 104 | ++ | B |
| 105 | 105 | +++ | A |
| 106 | 106 | +++ | B |

TEAD1-Luc MCF7: IC₅₀:
++++ ≤ 10 nM
+++ > 10 nM ≤ 100 nM
++ > 100 nM ≤ 500 nM
+ > 500 nM
TEAD1-Luc MCF7: % max. inhibition:
A ≥ 75%
75% > B ≥ 50%
50% > C ≥ 2%

### Biological Example 2

### Proliferation and Viability Assays

Compound potency and efficacy in inhibiting cell proliferation and viability was assessed in multiple cell lines. For example, NCI-H226 cells (ATCC #CRL-5826) were seeded at 2000 cells in 200 μL medium per well of a 96-well plate (Corning #3610) and incubated at 37° C. for 24 hours, then treated with serial diluted compounds for up to 6 d. Viable cells were measured by CellTiter-Glo 2.0 Cell Viability Assay (Promega #G9243), and the luminescence signal was read using an EnSight Multimode plate reader (PerkinElmer, model 9MLDHH34000000, integration time 100 ms) with Kaleido 3.0. software. For data analysis, dose-dependent curves were plotted as “inhibition” (% of DMSO-treated control wells), and were fitted by four-parametric model in GraphPad Prism to obtain IC₅₀ values.

Alternatively, cells were plated at 2000 cells in 200 μL medium per well of a 96-well plate (Corning #3585) overnight, treated with compound for 7 days, cell density images taken by Incycte S3 live cell analysis instrument (Sartorius) every 24 hours at the same time. Images were analyzed by Incucyte's AI-driven software to get C “% confluence” for every image. Dose-dependent curves were plotted as “% inhibition” (% o of DMSO-treated control wells) and were fitted by four-parametric model in GraphPad Prism to obtain IC₅₀ values.

TABLE 3
The pharmacological data obtained from Cells
Proliferation and viability assays

| | | CPA | CPA | CPA | CPA |
|---|---|---|---|---|---|
| | | NCI-H226 | NCI-H226 | H2052 | H2052 |
| | | IC50 | Max Inh | IC50 Ave | Max Inh |
| Example | Compound | Ave (nM) | Ave (%) | (nM) | Ave (%) |
| 1 | 1 | ++ | B | +++ | B |
| 9 | 9 | + | B | ++ | B |
| 10 | 10 | +++ | B | +++ | B |
| 11 | 11 | ++ | A | +++ | A |
| 12 | 12 | + | B | ++ | B |
| 13 | 13 | +++ | B | +++ | B |
| 14 | 14 | + | C | ++ | B |
| 15 | 15 | ++ | B | +++ | B |
| 21 | 21 | ++ | A | +++ | B |
| 34 | 34 | ++ | A | +++ | A |
| 36 | 36 | ++ | B | +++ | B |
| 38 | 38 | +++ | B | +++ | B |
| 45 | 45 | ++ | B | ++ | B |
| 59 | 59 | +++ | A | ++ | B |
| 60 | 60 | +++ | A | +++ | A |
| 61 | 61 | +++ | B | +++ | A |
| 67 | 67 | +++ | A | +++ | A |
| 85 | 85 | ++ | B | +++ | B |

CPA NCI-226 IC₅₀ Ave and CPA H2052 IC₅₀ Ave: +++ ≤ 100 nM; ++ 100-500 nM; + > 500 nM.
CPA NCI-H226 Max Inh Ave and CPA H2052 Max Inh Ave: A ≥ 75%; 75% > B ≥ 50%; 50% > C ≥ 45%.

### Biological Example 3

### TEAD Selectivity Assays

Tagged TEAD constructs were cloned in pTriEx-6 (Novagen #71559) and transiently transfected into HEK293T cells for 24 hours. Then cells were treated with compounds for 24 hours, or treated with compounds plus 25 μM alkyne palmitic acid (for palmitoylation assay), and cell pellets were collected. Cells were lysed in lysis Buffer (100 mM Tris-HCl, 150 mM NaCl, 1% Triton, 5 mg/mL protease inhibitor cocktail), centrifuged for 10 min at 15000 g, TEAD proteins were pulled down and subjected to click-chemistry with TAMRA azide (Click Chemistry Tools #AZ109) to label compound-modified protein. Protein bands were separated by SDS-gel electrophoresis, target bands at expected molecular size were visualized using BioRad ChemiDoc Imaging system, band intensities were quantified by Image Studio.

ELISA: 750k cells were plated per well of 6-well plate for 24 hours, treated with compounds overnight, cells were then lysed in lysis Buffer with 5 mg/mL protease inhibitor (Cell Signaling #9803), centrifuged at 12000 g for 5 min. Cell lysates were normalized to 2 mg/mL for subsequent assays. A 96-well assay plate (Corning #3690) was coated with TEAD antibodies (anti-TEAD1: Cell Signaling #12292, 1:100; anti-TEAD2: LSBio #LS-C101693-100, 1:50; anti-TEAD3: Abcam #ab75192, 1:100; anti-TEAD4: Abcam #ab58310, 1:400) overnight at 4° C. Plates were washed with TBST (Thermo Fisher #28360) for 3 times, blocked in 100 μL blocking buffer (2% BSA-TBST, Sigma #A7030) for 1 hour at room temperature. 50 μL cell lysates were incubated overnight at 4° C. with shaking. The next day, plates were washed with TBST three times, incubated with 1:125 anti-YPA-HRP (Cell Signaling #15028S) for 4 hours at 4° C., followed by 3× washing in TBST, then incubated in HRP substrate (Thermo Fisher #34021) for 10 min at room temperature, reaction was stopped by TMB stop solution (Thermo Fisher #N600). Signal was read at wavelength 450 nm.

TABLE 4
Palmitoylation assay of compounds (3 um) in TEAD proteins

| | | TEAD1 | TEAD2 | TEAD3 | TEAD4 |
|---|---|---|---|---|---|
| Example | Compound | Inh(%) | Inh(%) | Inh(%) | Inh(%) |
| 10 | 10 | 94 | 57 | 98 | 52 |
| 15 | 15 | 97 | 80 | 98 | 80 |
| 21 | 21 | 96 | 70 | 96 | 33 |
| 38 | 38 | 93 | 69 | 95 | 71 |
| 39 | 39 | 89 | 32 | 90 | 18 |
| 83 | 83 | 93 | 54 | 90 | 52 |

TABLE 5
ELLISA assay of compounds in H226 Cells

| | | TEAD1 | TEAD2 | TEAD3 | TEAD4 |
|---|---|---|---|---|---|
| Example | Compound | IC₅₀ (nM) | IC₅₀ (nM) | IC₅₀ (nM) | IC₅₀ (nM) |
| 10 | 10 | 13 | 89 | 7.2 | 1009 |
| 15 | 15 | 14 | 179 | 4 | >10000 |
| 38 | 38 | 8.7 | 3.4 | 5.6 | 15.5 |
| 83 | 83 | 66.4 | 17.7 | 107 | 219 |

### Biological Example 4

### Pharmacokinetics of Compounds in Plasma Following PO Dosing in Mice

Adult Balb/C female mice (6-8 weeks, Vital River Laboratory) for PO group will be food fasted overnight and be fed 2 h after dosing. They will have free access to water. Mice were oral dosed test compounds A, B, or C, compounds of Formula (I) in the vehicle of 5% DMSO, 10% Solutol and 85% D5W (freshly prepared on the day of dosing). The mice (n=3) were giving oral administration at a dose of 10 mg/kg. Time of blood collection: 0.25, 0.5, 1, 2, 4, 8 and 24 hours post dose. About 0.03 mL of whole blood was collected per time point from Dorsal metatarsal vein, and placed into tubes that contained EDTA as an anticoagulant. The samples were centrifuged at 4° C., and 4000 rpm for 5 min. The plasma samples will be stored in a freezer at −75±15° C. prior to analysis. Concentrations of test compounds in the plasma samples were analyzed with liquid chromatography-tandem mass spectrometry (LC-MS/MS). WinNonlin (Phoenix™, version 8.3) or other similar software was used for pharmacokinetic calculations. PK data of some compounds are shown in Table 6.

TABLE 6
PK Data of Some compounds

| | Cmax | Auc |
|---|---|---|
| Compound | (ng/mL) | (h*ng/mL) |
| A | 1489 | 1742 |
| B | 1943 | 3949 |
| C | 3617 | 15451 |

### Biological Example 5

### In Vivo Efficacy Study

In vivo efficacy studies of after administration of compound B or C, compounds of Formula (I), in female NOD SCID mice bearing NCI-H226 xenografts.

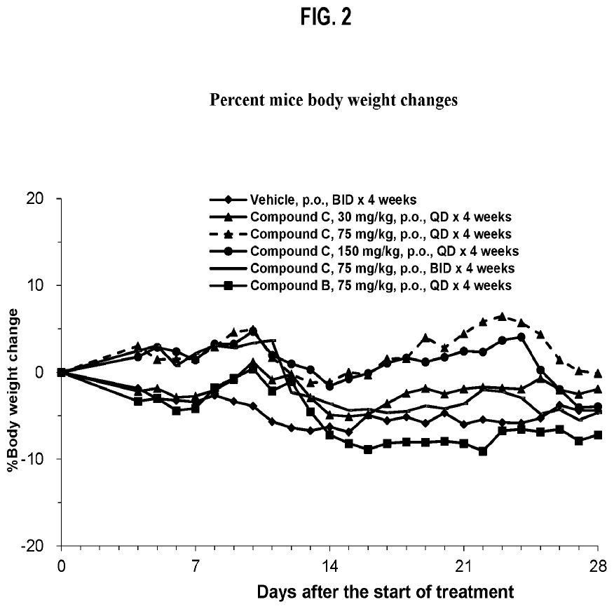

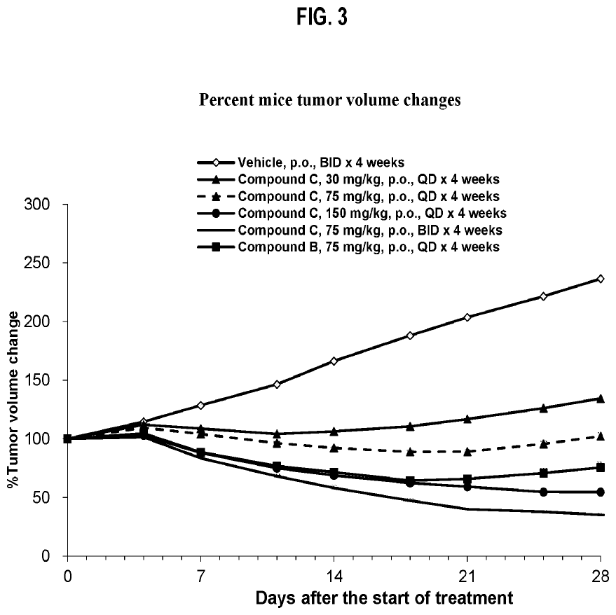

Method: Each mouse (female NOD SCID mice, 6 to 8 weeks, Vital River laboratory) was inoculated subcutaneously at the right flank with NCI-H226 tumor cells (10×106) in 0.2 mL of PBS supplemented with matrigel (PBS:Matrigel=1:1) for tumor development. Treatments started on day 41 after tumor inoculation, when the average tumor size reached 150 mm³. Each group contained 5 mice. The test compounds were administrated in the formulation of (5% DMSO+10% Solutol+85% D5W) to the mice from the day of grouping for total 28 days. Animal body weight was monitored regularly as an indicator measure of toxicity. Tumor volumes were measured twice weekly in two dimensions using a caliper, and the tumor volume (mm³) was calculated using formula V=0.5 a×b² where a and b are the long and short diameters of the tumor in mm, respectively. Mice were randomized into different treatment groups based on the tumor volume. Therapeutic efficacy was evaluated by tumor growth inhibition TGI (%) using the formula: TGI (%)=[1−(Ti−T0)/(Ci−C0)]×100; Ti is the average tumor volume of a treatment group on a given day, T0 is the average tumor volume of the treatment group on the day of treatment start, Ci is the average tumor volume of the control group on the same day with Ti, and C0 is the average tumor volume of the vehicle group on the day of treatment start. Antitumor activity T/C (%) was determined by dividing the average tumor volume for treated group (T) by the average tumor volume for control group (C). A one-way ANOVA was performed to compare tumor volume among vehicle group and treatment groups by p value. The results are shown in Table 7, FIG. 2 and FIG. 3.

TABLE 7
Tumor Growth Inhibition Analysis (T/C and TGI) based on
tumor volume measured on Per Group at Day 28 (PG-D 28).

| | Tumor Size | | | |
|---|---|---|---|---|
| | (mm³)^{a} | | | p^{b} |
| Group | at PG-D 28 | T/C(%) | TGI | value |
| Vehicle, p.o., BID | 356 ± 44 | — | — | — |
| Compound C, | 200 ± 9 | 56.2 | 75.5 | **** |
| 30 mg/kg, p.o., QD | | | | |
| Compound C, | 154 ± 11 | 43.2 | 98.0 | **** |
| 75 mg/kg, p.o., QD | | | | |
| Compound C, | 85 ± 11 | 23.8 | 131.4 | **** |
| 150 mg/kg, p.o., QD | | | | |
| Compound C, | 53 ± 4 | 14.9 | 147.0 | **** |
| 75 mg/kg, p.o., BID | | | | |
| Compound B, | 113 ± 9 | 31.9 | 117.8 | **** |
| 75 mg/kg, p.o., QD | | | | |

^{a}Data are shown as Mean ± SEM;
^{b}**** indicates p < 0.0001.

On day 28 post treatment, compound C group (30 mg/kg, QD), compound C group (75 mg/kg, QD), compound C group (150 mg/kg, QD), compound C group (75 mg/kg, BID), compound B group (75 mg/kg, QD) showed significant anti-tumor activities compared with the vehicle group in tumor volume. The p values in all groups were less than 0.0001. T/C (%) values are 56.2%, 43.2%, 23.8%, 14.9% and 31.9%, respectively. TGI (%) values were 75.5%, 98.0%, 131.4%, 147.0%, and 117.8%, respectively.

In this model, as shown in FIG. 2, no significant body weight loss was observed during the treatment with compound B and C with different doses. Percent tumor volume change is shown in FIG. 3.

We claim:

1. A compound of formula (I); 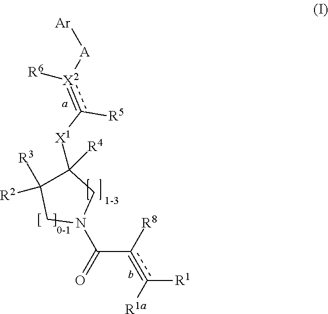
or a stereoisomer, and/or a pharmaceutically acceptable salt, and/or solvate thereof, wherein:
bond b is a double bond or triple bond;
R¹ is selected from the group consisting of H, C₁₋₃alkyl, morpholin-4-yl, and —CH₂N(C₁₋₃ alkyl)₂;
R^{1a} is absent when bond b is a triple bond; and R^{1a} is H when bond b is a double bond;
R² is selected from the group consisting of H, F, —OH, C₁₋₃alkyl, —OC₁₋₃alkyl, —NH-heteroaryl, heteroaryl, —NH-(substituted heteroaryl), and substituted heteroaryl;
R³ is selected from the group consisting of H and C₁₋₃alkyl; and
R⁸ is absent when bond b is a triple bond; and R⁸ is selected from the group consisting of H, F, CN, and C₁₋₃alkyl when bond b is a double bond; and
i) wherein bond a, X¹, X², R⁴, R⁵, R⁶, A, and Ar are as follows:
bond a is a double bond or triple bond;
X¹ is absent or is selected from the group consisting of O, CH(OH), CH(OCH₃), CH₂, CH₂CH₂, CH(CH₃), and C(CH₃)₂, and R⁴ is selected from the group consisting of H, C₁-C₄ alkyl, cyclopropyl, F, Cl, —OH, —OC₁₋₃alkyl, CN, and NHR⁷, and R⁷ is selected from the group consisting of H and C1-C₃ alkyl; or X¹ and R⁴ together with the carbon to which they are attached form C₄-C₆ cycloalkylene;
X² is X^{2a} and X^{2a} is C;
R⁵ is absent when bond a is a triple bond; or R⁵ is selected from the group consisting of H, F, and C₁-C₃ alkyl when bond a is a double bond.
R⁶ is absent when bond a is a triple bond; or R⁶ is selected from the group consisting of H, F, and C₁-C₃ alkyl when bond a is a double bond;
A is absent or selected from the group consisting of O, CH₂, CH₂CH₂, CH(OH), CH(OCH₃), C(CH₃)₂ and CH(CH₃); and
Ar is aryl or heteroaryl, each of which is optionally substituted with 1, 2, 3, or 4 R⁹ groups; or
ii) wherein bond a, X¹, X², R⁴, R⁵, R⁶, A, and Ar together form: 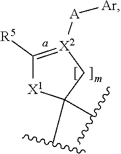
and
X² is X^{2a} and X^{2a} is C; R⁴ and R⁶ together form —(CH₂)ₘ—; m is 0, 1, or 2; X¹ is absent or is selected from the group consisting of CH₂, CH₂CH₂, CH(CH₃), O, CH₂O, and OCH₂; R⁵ is selected from the group consisting of H, F, and C₁-C₃ alkyl; A is absent or selected from the group consisting of O, CH₂, CH₂CH₂, and CH(CH₃); and Ar is aryl or heteroaryl, each of which is optionally substituted with 1, 2, 3, or 4 R⁹ groups; or
iii) wherein bond a, X¹, X², R⁴, R⁵, R⁶, A, and Ar together form: 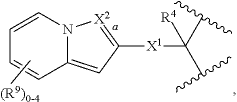
and
X¹ is absent or is selected from the group consisting of O, CH₂, CH₂CH₂, and CH(CH₃); X² is X^{2b} and X^{2b} is N; R⁴ is selected from the group consisting of H, C₁-C₄ alkyl, cyclopropyl, F, Cl, —OH, —OC₁₋₃alkyl, CN, and NHR⁷, and R⁷ is selected from the group consisting of H and C₁-C₃ alkyl; R⁵ and Ar together form 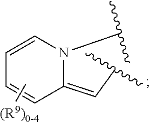
and R⁶ and A are absent; or
iv) wherein bond a, X, X², R⁴, R⁵, R⁶, A, and Ar together form: 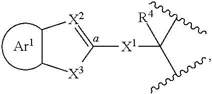
and
X¹ is absent or is selected from the group consisting of O, CH₂, CH₂CH₂, and CH(CH₃); X² is X²c and X², is N or CR⁶, and R⁶ is selected from the group consisting of H, F, and C₁-C₃ alkyl; R⁴ is selected from the group consisting of H, C₁-C₄ alkyl, cyclopropyl, F, Cl, —OH, —OC₁₋₃alkyl, CN, and NHR⁷, and R⁷ is selected from the group consisting of H and C₁-C₃ alkyl; A is absent; and R⁵ and Ar together form 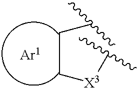
where X³ is O, NH, N(C₁-C₃ alkyl), and Ar¹ is a fused arylene or fused heteroarylene ring, where the Ar¹ ring is optionally substituted with 1, 2, 3, or 4 R⁹ groups; and
wherein each R⁹ is independently selected from the group consisting of C₁-C₆ alkyl, C₂-C₆ alkenyl, C2-C6 alkynyl, halo, C₁-C₆ haloalkyl, C₁-C₆ alkoxy, and CN; and
provided that the compound is not 1-[4-[(1E)-2-(2,6-dimethylphenyl)ethenyl]-1-piperidinyl]-2-propen-1-one.

2. The compound of claim 1, or a stereoisomer, and/or a pharmaceutically acceptable salt, and/or solvate thereof, wherein bond b is a double bond and R^{1a} is H.

3. The compound of claim 1, or a stereoisomer, and/or a pharmaceutically acceptable salt, and/or solvate thereof, wherein bond b is a triple bond and R^{1a} and R⁸ are absent.

4. The compound of any one of claims 1-3, or a stereoisomer, and/or a pharmaceutically acceptable salt, and/or solvate thereof, wherein R¹ is H.

5. The compound of any one of claims 1-3, or a stereoisomer, and/or a pharmaceutically acceptable salt, and/or solvate thereof, wherein R¹ is Me.

6. The compound of any one of claims 1-3, or a stereoisomer, and/or a pharmaceutically acceptable salt, and/or solvate thereof, wherein R¹ is selected from the group consisting of H and Me.

7. The compound of any one of claims 1-6, or a stereoisomer, and/or a pharmaceutically acceptable salt, and/or solvate thereof, wherein R² is H.

8. The compound of any one of claims 1-6, or a stereoisomer, and/or a pharmaceutically acceptable salt, and/or solvate thereof, wherein R² is F.

9. The compound of any one of claims 1-6, or a stereoisomer, and/or a pharmaceutically acceptable salt, and/or solvate thereof, wherein R² is C₁₋₃alkyl.

10. The compound of any one of claims 1-6, or a stereoisomer, and/or a pharmaceutically acceptable salt, and/or solvate thereof, wherein R² is H, methyl or ethyl.

11. The compound of any one of claims 1-6, or a stereoisomer, and/or a pharmaceutically acceptable salt, and/or solvate thereof, wherein R² is —OC₁₋₃alkyl.

12. The compound of any one of claims 1-6 and 11, or a stereoisomer, and/or a pharmaceutically acceptable salt, and/or solvate thereof, wherein R² is —OMe.

13. The compound of any one of claims 1-6, or a stereoisomer, and/or a pharmaceutically acceptable salt, and/or solvate thereof, wherein R² is —NH-heteroaryl or —NH-(substituted heteroaryl).

14. The compound of any one of claims 1-6, or a stereoisomer, and/or a pharmaceutically acceptable salt, and/or solvate thereof, wherein R² is unsubstituted heteroaryl (in some embodiments, pyrazolyl) or substituted heteroaryl (in some embodiments, pyrazolyl).

15. The compound of any one of claims 1-6, or a stereoisomer, and/or a pharmaceutically acceptable salt, and/or solvate thereof, wherein R² is H, methyl, ethyl, methoxy, —NH-heteroaryl (in some embodiments, —NH-pyrimidinyl), or unsubstituted heteroaryl (in some embodiments, pyrazolyl).

16. The compound of any one of claims 1-15, or a stereoisomer, and/or a pharmaceutically acceptable salt, and/or solvate thereof, wherein R³ is H.

17. The compound of any one of claims 1-15, or a stereoisomer, and/or a pharmaceutically acceptable salt, and/or solvate thereof, wherein R³ is methyl or ethyl.

18. The compound of any one of claims 1, 2, and 4-17, or a stereoisomer, and/or a pharmaceutically acceptable salt, and/or solvate thereof, wherein bond b is a double bond and R⁸ is H.

19. The compound of any one of claims 1, 2, and 4-17, or a stereoisomer, and/or a pharmaceutically acceptable salt, and/or solvate thereof, wherein bond b is a double bond and R⁸ is F.

20. The compound of any one of claims 1-19, or a stereoisomer, and/or a pharmaceutically acceptable salt, and/or solvate thereof, wherein bond a, X¹, X², R⁴, R⁵, R⁶, A, and Ar are according to i).;

21. The compound of claim 20, or a stereoisomer, and/or a pharmaceutically acceptable salt, and/or solvate thereof, wherein X¹ is absent, CH₂, CH(OH), or C(CH₃)₂.

22. The compound of claim 20 or 21, or a stereoisomer, and/or a pharmaceutically acceptable salt, and/or solvate thereof, wherein X¹ is absent.

23. The compound of claim 20 or 21, or a stereoisomer, and/or a pharmaceutically acceptable salt, and/or solvate thereof, wherein X¹ is CH₂.

24. The compound of claim 1, according to the following formula: 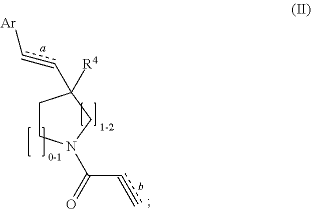
or a stereoisomer, and/or a pharmaceutically acceptable salt, and/or solvate thereof.

25. The compound of any one of claims 20-24, or a stereoisomer, and/or a pharmaceutically acceptable salt, and/or solvate thereof, wherein R⁴ is H.

26. The compound of any one of claims 20-24, or a stereoisomer, and/or a pharmaceutically acceptable salt, and/or solvate thereof, wherein R⁴ is C₁-C₄ alkyl (methyl).

27. The compound of any one of claims 20-24, or a stereoisomer, and/or a pharmaceutically acceptable salt, and/or solvate thereof, wherein R⁴ is F.

28. The compound of any one of claims 20-24, or a stereoisomer, and/or a pharmaceutically acceptable salt, and/or solvate thereof, wherein R⁴ is —OH.

29. The compound of any one of claims 20-24, or a stereoisomer, and/or a pharmaceutically acceptable salt, and/or solvate thereof, wherein R⁴ is —OMe.

30. The compound of any one of claims 20-24, or a stereoisomer, and/or a pharmaceutically acceptable salt, and/or solvate thereof, wherein R⁴ is CN.

31. The compound of claim 20, or a stereoisomer, and/or a pharmaceutically acceptable salt, and/or solvate thereof, wherein X¹ and R⁴ together with the carbon to which they are attached form a C₄-C₆ cycloalkylene.

32. The compound of claim 20 or 31, or a stereoisomer, and/or a pharmaceutically acceptable salt, and/or solvate thereof, wherein X¹ and R⁴ together with the carbon to which they are attached form a C₄ cycloalkylene or C₆ cycloalkylene.

33. The compound of any one of claims 20-32, or a stereoisomer, and/or a pharmaceutically acceptable salt, and/or solvate thereof, wherein bond a is a double bond.

34. The compound of any one of claims 20-33, or a stereoisomer, and/or a pharmaceutically acceptable salt, and/or solvate thereof, wherein R⁵ and R⁶ are each H.

35. The compound of any one of claims 20-34, or a stereoisomer, and/or a pharmaceutically acceptable salt, and/or solvate thereof, wherein bond a is cis.

36. The compound of any one of claims 20-34, or a stereoisomer, and/or a pharmaceutically acceptable salt, and/or solvate thereof, wherein bond a is trans.

37. The compound of any one of claims 20-32, or a stereoisomer, and/or a pharmaceutically acceptable salt, and/or solvate thereof, wherein bond a is a triple bond and R⁵ and R⁶ are absent.

38. The compound of any one of claims 1-19, or a stereoisomer, and/or a pharmaceutically acceptable salt, and/or solvate thereof, wherein bond a, X¹, X², R⁴, R⁵, R⁶, A, and Ar are according to ii).

39. The compound of claim 38, or a stereoisomer, and/or a pharmaceutically acceptable salt, and/or solvate thereof, wherein m is 1.

40. The compound of claim 38, or a stereoisomer, and/or a pharmaceutically acceptable salt, and/or solvate thereof, wherein m is 2.

41. The compound of any one of claims 38-40, or a stereoisomer, and/or a pharmaceutically acceptable salt, and/or solvate thereof, wherein X¹ is absent.

42. The compound of claim 38, or a stereoisomer, and/or a pharmaceutically acceptable salt, and/or solvate thereof, wherein m is 0 and X¹ is other than absent.

43. The compound of any one of claims 38-40 and 42, or a stereoisomer, and/or a pharmaceutically acceptable salt, and/or solvate thereof, wherein X¹ is CH₂, CH₂CH₂, CH(CH₃), O, CH₂O, or OCH₂.

44. The compound of any one of claims 38-43, or a stereoisomer, and/or a pharmaceutically acceptable salt, and/or solvate thereof, wherein R⁵ is H.

45. The compound of any one of claims 20-44, or a stereoisomer, and/or a pharmaceutically acceptable salt, and/or solvate thereof, wherein A is absent.

46. The compound of any one of claims 20-37, or a stereoisomer, and/or a pharmaceutically acceptable salt, and/or solvate thereof, wherein A is selected from the group consisting of CH₂, CH(OH), CH(OCH₃), and C(CH₃)₂.

47. The compound of any one of claims 20-46, or a stereoisomer, and/or a pharmaceutically acceptable salt, and/or solvate thereof, wherein Ar is phenyl optionally substituted with 1, 2, 3, or 4 R⁹ groups (in some embodiments, the phenyl is optionally substituted with 1 R⁹).

48. The compound of any one of claims 20-46, or a stereoisomer, and/or a pharmaceutically acceptable salt, and/or solvate thereof, wherein Ar is heteroaryl, (preferably oxazolyl, imidazolyl, or pyridinyl), optionally substituted with 1, 2, 3, or 4 R⁹ groups (in some embodiments, the heteroaryl is optionally substituted with 1 R⁹).

49. The compound of any one of claims 1-19, or a stereoisomer, and/or a pharmaceutically acceptable salt, and/or solvate thereof, wherein bond a, X¹, X², R⁴, R⁵, R⁶, A, and Ar are according to iii).

50. The compound of claim 49, or a stereoisomer, and/or a pharmaceutically acceptable salt, and/or solvate thereof, wherein X¹ is absent, and R⁴ is H.

51. The compound of any one of claims 1-19, or a stereoisomer, and/or a pharmaceutically acceptable salt, and/or solvate thereof, wherein bond a, X¹, X², R⁴, R⁵, R⁶, A, and Ar are according to iv).

52. The compound of claim 51, or a stereoisomer, and/or a pharmaceutically acceptable salt, and/or solvate thereof, wherein X² is CH.

53. The compound of any one of claims 51-52, or a stereoisomer, and/or a pharmaceutically acceptable salt, and/or solvate thereof, wherein X¹ is absent and R⁴ is H.

54. The compound of any one of claims 51-53, or a stereoisomer, and/or a pharmaceutically acceptable salt, and/or solvate thereof, wherein X³ is O, NH, N(Me).

55. The compound of any one of claims 51-54, or a stereoisomer, and/or a pharmaceutically acceptable salt, and/or solvate thereof, wherein Ar¹ is benzo or pyrido, optionally substituted with 1, 2, 3, or 4 R⁹ groups.

56. The compound of any one of claims 1-55, or a stereoisomer, and/or a pharmaceutically acceptable salt, and/or solvate thereof, wherein each R⁹ is independently selected from the group consisting of C₁-C₆ alkyl, C₂-C₆ alkynyl, halo, C₁-C₆ haloalkyl, and C₁-C₆ alkoxy; or each R⁹ is independently selected from the group consisting of methyl, ethynyl, F, CF₃, and methoxy.

57. The compound of any one of claims 1-56, or a stereoisomer, and/or a pharmaceutically acceptable salt, and/or solvate thereof, wherein 1 or 2 R⁹ are present and are independently selected.

58. The compound of any one of claims 1-57, or a stereoisomer, and/or a pharmaceutically acceptable salt, and/or solvate thereof, wherein one R⁹ is CF₃.

59. The compounds of any one of claims 1-58, or a stereoisomer, and/or a pharmaceutically acceptable salt, and/or solvate thereof, wherein 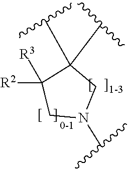
is

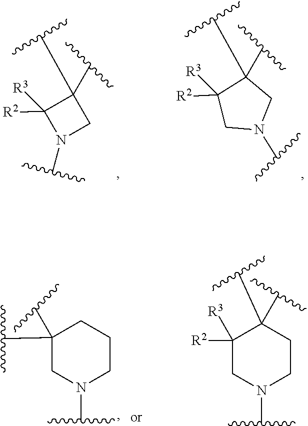

60. The compound of claim 1, or a stereoisomer, and/or a pharmaceutically acceptable salt, and/or solvate thereof, selected from the compounds in Table 1.

61. The compound of claim 1, or a stereoisomer, and/or a pharmaceutically acceptable salt, and/or solvate thereof, selected from the group consisting of:

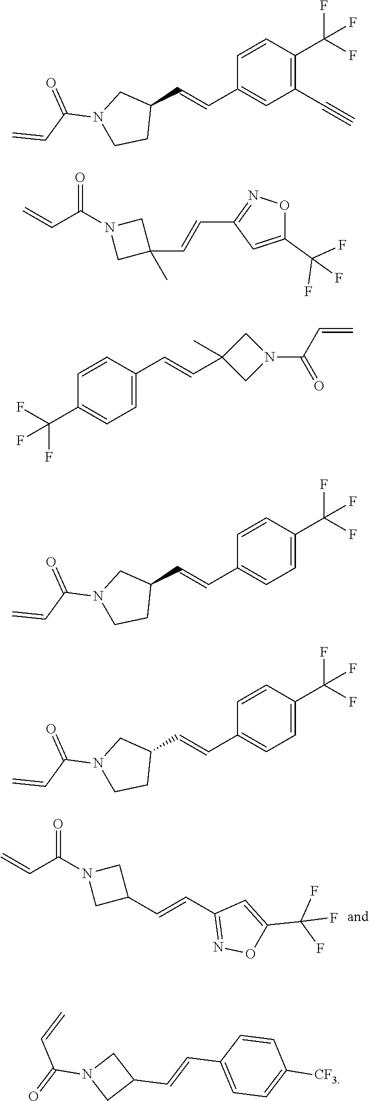

62. A pharmaceutical composition, comprising a pharmaceutically effective amount of the compound according to any one of claims 1-61 or a stereoisomer, and/or a pharmaceutically acceptable salt, and/or solvate thereof; and a pharmaceutically acceptable carrier.

63. A method of treating cancer in a subject in need thereof, comprising administering to the subject a therapeutically effective amount of the compound of any one of claims 1-61, or a stereoisomer, and/or a pharmaceutically acceptable salt, and/or solvate thereof; or the pharmaceutical composition of claim 62.

64. The method of claim 63, wherein the cancer is selected from the group consisting of bladder cancer, breast cancer, ovarian cancer, pancreatic ductal adenocarcinoma (PDAC), glioblastoma, gastric cancer, cervical cancer, colon cancer, endometrial cancer, head and neck cancer, lung cancer, melanoma, multiple myeloma, leukemia, non-Hodgkin's lymphoma, prostate cancer, rectal cancer, malignant melanomas, alimentary/gastrointestinal tract cancer, liver cancer, skin cancer, lymphoma, malignant pleural mesothelioma (MPM), kidney cancer, muscle cancer, bone cancer, brain cancer, eye or ocular cancer, rectal cancer, colorectal cancer, cervical cancer, oral cancer, benign and malignant tumors, stomach cancer, corpus uteri, testicular cancer, renal cancer, throat cancer, acute lymphocytic leukemia, acute myelogenous leukemia, Ewing's Sarcoma, Kaposi's Sarcoma, basal cell carcinoma and squamous cell carcinoma, small cell lung cancer, choriocarcinoma, rhabdomyosarcoma, angiosarcoma, hemangioendothelioma, Wilms Tumor, neuroblastoma, mouth/pharynx cancer, esophageal cancer, larynx cancer, neurofibromatosis, tuberous sclerosis, hemangiomas, and lymphangiogenesis.

65. The method of claim 63, wherein the cancer is selected from the group consisting of glioblastoma, gastric cancer, colorectal cancer, pancreatic ductal adenocarcinoma (PDAC), and malignant pleural mesothelioma (MPM).

66. The method of any one of claims 63-65, wherein the method further comprises simultaneously or sequentially administering one or more additional anti-cancer therapies, or
one or more KRAS G12C and/or G12D inhibitors; one or more CDK4/6 inhibitors;
one or more EGFR inhibitors; one or more RAF inhibitors; one or more MEK inhibitors; one or more Wnt signaling inhibitors, such as anti-β-catenin inhibitors, GSK3 inhibitors, JNK inhibitors, and CKT inhibitors; one or more TGF-β signaling inhibitors, such as atezolizumab, durvalumab, and avelumab; one or more PD-1/PD-L1 inhibitors; and/or radiotherapy; or
one or more additional therapies comprises a KRAS G12C and/or G12D inhibitor, or the one or more KRAS G12C and/or G12D inhibitor is sotorasib or adagrasib.

67. A method of inhibiting TEAD1, 2, 3, and/or 4 in a subject or in a sample, comprising administering to the subject a therapeutically effective amount of the compound of any one of claims 1-61, or a stereoisomer, and/or a pharmaceutically acceptable salt, and/or solvate thereof; or the pharmaceutical composition of claim 62.

68. The method of claim 67, wherein TEAD1 is inhibited selectively over TEAD2, 3, and 4.
